(19) United States
(12) Patent Application Publication
(10) Pub. No.: US 2022/0380758 A1
(43) Pub. Date: Dec. 1, 2022

(54) TYPE I-B CRISPR-ASSOCIATED TRANSPOSASE SYSTEMS

(71) Applicants: THE BROAD INSTITUTE, INC., Cambridge, MA (US); MASSACHUSETTS INSTITUTE OF TECHNOLOGY, Cambridge, MA (US)

(72) Inventors: Makoto Saito, Cambridge, MA (US); Feng Zhang, Cambridge, MA (US); Guilhem Faure, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/773,104

(22) PCT Filed: Oct. 30, 2020
(86) PCT No.: PCT/US2020/058405

§ 371 Date: Apr. 29, 2022

Related U.S. Application Data
(60) Provisional application No. 62/929,621, filed on Nov. 1, 2019.
(60) Provisional application No. 62/964,010, filed on Jan. 21, 2020.
(60) Provisional application No. 62/980,897, filed on Feb. 24, 2020.
(60) Provisional application No. 63/046,470, filed on Jun. 30, 2020.
(60) Provisional application No. 63/104,442, filed on Oct. 22, 2020.

(51) Int. Cl. C12N 15/11; C12N 15/10; C12N 15/63

(52) U.S. Cl. CPC C12N 15/11; C12N 15/102; C12N 15/63; C12N 2310/20

(57) ABSTRACT

Systems and methods for targeted gene modification, targeted insertion, perturbation of gene transcripts, and nucleic acid editing. Novel nucleic acid targeting systems comprise components of CRISPR systems and transposable elements.

### CROSS-REFERENCE TO RELATED APPLICATIONS

This application claims the benefit of U.S. Provisional Application Nos. 62/929,621, filed Nov. 1, 2019; 62/964,010, filed Jan. 21, 2020; 62/980,897, filed Feb. 24, 2020; 63/046,470, filed Jun. 30, 2020; and 63/104,442, filed Oct. 22, 2020. The entire contents of the above-identified applications are hereby fully incorporated herein by reference.

### STATEMENT REGARDING FEDERALLY SPONSORED RESEARCH

This invention was made with government support under Grant No. HL141201 awarded by the National Institutes of Health. The government has certain rights in the invention.

### REFERENCE TO AN ELECTRONIC SEQUENCE LISTING

The contents of the electronic sequence listing (“BROD-4890WP_ST25.txt”; Size is 426,096 bytes and it was created on Oct. 30, 2020) is herein incorporated by reference in its entirety.

### TECHNICAL FIELD

The subject matter disclosed herein is generally directed to systems, methods and compositions used for targeted gene modification, targeted insertion, perturbation of gene transcripts, and nucleic acid editing. Novel nucleic acid targeting systems comprise components of Clustered Regularly Interspaced Short Palindromic Repeats (CRISPR) systems and transposable elements.

### BACKGROUND

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ novel strategies and molecular mechanisms and are affordable, easy to set up, scalable, and amenable to targeting multiple positions within the eukaryotic genome. This would provide a major resource for new applications in genome engineering and biotechnology.

The CRISPR-Cas systems of bacterial and archaeal adaptive immunity show extreme diversity of protein composition, genomic loci architecture, and system function. Systems comprising CRISPR-like components are widespread and continue to be discovered. Novel multi-subunit effector complexes and single-subunit effector modules may be developed as powerful genome engineering tools.

Citation or identification of any document in this application is not an admission that such document is available as prior art to the present invention.

### SUMMARY

In one aspect, the present disclosure provides an engineered system, the system comprising: one or more CRISPR-associated Tn7 or Tn7-like transposases or functional fragments thereof; one or more Type I-B Cas proteins; and a guide molecule capable of complexing with the Type I-B Cas protein and directing binding of the guide-Cas protein complex to a target polynucleotide.

In some embodiments, the one or more CRISPR-associated Tn7 or Tn7-like transposases comprise TnsA, TnsB, TnsC, and/or TniQ. In some embodiments, the Tn7 or Tn7-like transposases comprise TnsA, TnsB, TnsC, a first TniQ and a second TniQ, wherein the first and second TniQ are different. In some embodiments, the Tn7 or Tn7-like transposases comprise comprises TnsA, TnsB, TnsC, and a TniQ. In some embodiments, the TniQ comprises a DNA-binding domain. In some embodiments, the DNA-binding domain is at C-terminus of the TniQ. In some embodiments, the one or more CRISPR-associated Tn7 or Tn7-like transposases comprise TnsA, TnsB, TnsC, and/or TnsD. In some embodiments, the Tn7 or Tn7-like transposases comprise TnsA, TnsB, TnsC, a first TnsD and a second TnsD, wherein the first and second TniD are different. In some embodiments, the Tn7 or Tn7-like transposase comprises TnsA, TnsB, TnsC and a TnsD. In some embodiments, the TnsD comprises a DNA-binding domain. In some embodiments, the TnsA and TnsB are comprised in a single protein. In some embodiments, the Tn7 or Tn7-like transposase comprises TnsA, TnsB, TnsC and TnsD1; TnsA, TnsB, TnsC and TnsD2; or TnsA, TnsB, TnsC, TnsD, and TnsD2.

In some embodiments, the one or more Type I-B Cas proteins comprises Cas5, Cas6, Cas7, and/or Cas 8. In some embodiments, the one or more Type I-B Cas proteins comprises Cas 8b. In some embodiments, the Cas8b is Cas8b3.

In some embodiments, the one or more CRISPR-associated Tn7 or Tn7-like transposases and/or the one or more Type I-B Cas proteins are from or originated from Anabaena variabilis. In some embodiments, the one or more Type I-B Cas proteins lacks nuclease activity. In some embodiments, the system further comprises a donor polynucleotide. In some embodiments, the donor polynucleotide comprises a polynucleotide insert, a left element sequence, and a right element sequence. In some embodiments, the donor polynucleotide introduces one or more mutations to the target polynucleotide, corrects a premature stop codon in the target polynucleotide, disrupts a splicing site, restores a splicing site, or a combination thereof. In some embodiments, the one or more mutations introduced by the donor polynucleotide comprises substitutions, deletions, insertions, or a combination thereof. In some embodiments, the one or more mutations causes a shift in an open reading frame on the target polynucleotide. In some embodiments, the donor polynucleotide is between 100 bases and 30 kb in length. In some embodiments, the target polynucleotide comprises a protospacer adjacent motif (PAM) on 5′ side of the target polynucleotide. In some embodiments, the PAM is AT or ATG. In some embodiments, the system further comprises a targeting moiety. In some embodiments, the system comprises a plurality of guide molecules capable of directing binding of the guide-Cas protein complex to one or more target polynucleotides.

In some embodiments, the donor polynucleotide is inserted to the target polynucleotide at a site no more than 80, no more 90, no more 100, no more 200, or no more 300 bp 3′ of the PAM. In some embodiments, donor polynucleotide is inserted to the target polynucleotide at a site no more than 80, no more 90, no more 100, no more 200, or no more 300 bp 5′ of the PAM. In some embodiments, the target polynucleotide is linear, circular, or genomic DNA. In some embodiments, the one or more Tn7 transposase is derived from a first species and the one or more Type I-B Cas proteins is derived from a second species different from the first species. In some embodiments, the system comprises a first TnsD1 derived from the first species and a second TnsD2 derived from the second species. In some embodiments, the one or more Tn7 transposases comprises a transposase with activity of TnsA and TnsB.

In another aspect, the present disclosure provides a system comprising one or more polynucleotides encoding: one or more CRISPR-associated Tn7 or Tn7-like transposases or functional fragments thereof, one or more Type I-B Cas proteins; and a guide molecule capable of complexing with the Type I-B Cas protein and directing binding of the guide-Cas protein complex to a target polynucleotide.

In some embodiments, the system further comprises a donor polynucleotide. In some embodiments, the donor polynucleotide comprises a polynucleotide insert, a left element sequence, and a right element sequence. In some embodiments, the system comprises one or more polynucleotides or encoded products of the polynucleotides in one or more loci in Table 7. In some embodiments, the one or more polynucleotides encode components (a)-(c) of the system. In some embodiments, the one or more Type I-B Cas proteins comprises Cas5, Cas6, Cas7, and/or Cas 8. In some embodiments, the system comprises a first polynucleotide encoding a first Cas6 and a second polynucleotide encoding a second Cas6.

In another aspect, the present disclosure provides a vector comprising the one or more polynucleotides herein.

In another aspect, the present disclosure provides an engineered cell comprising the system herein, or the vector herein. In some embodiments, the cell produces and/or secretes an endogenous or non-endogenous biological product or chemical compound. In some embodiments, the biological product is a protein or an RNA.

In another aspect, the present disclosure provides a cell line comprising the engineered cell herein and progeny thereof.

In another aspect, the present disclosure provides a plant or animal model comprising the engineered cell herein and progeny thereof.

In another aspect, the present disclosure provides a composition comprising the engineered cell herein. In some embodiments, the composition is formulated for use as a therapeutic. In another aspect, the present disclosure provides a biological product or chemical compound produced by the engineered cell herein.

In another aspect, the present disclosure provides an engineered cell or progeny thereof, the cell being engineered by use of the system herein. In some embodiments, the cell or progeny thereof is isolated. In some embodiments, the cell or progeny thereof is further used as a therapeutic. In some embodiments, the cell or progeny thereof include those from which a product is isolated.

In another aspect, the present disclosure provides a product produced by the cell or progeny thereof herein. In some embodiments, the product is a protein or an RNA. In some embodiments, the protein comprises a mutation.

In another aspect, the present disclosure provides a pharmaceutical composition for treatment of a disease or disorder, comprising the cell or progeny thereof herein. In some embodiments, the treatment results in genetic changes in one or more cells. In some embodiments, the treatment results in correction of one or more defective genotypes. In some embodiments, the treatment results in improved phenotype. In some embodiments, the cell comprises a mutation in a protein expressed from a gene comprising the target sequence. In some embodiments, the cell comprises deletion of a genomic region comprising the target sequence. In some embodiments, the cell comprises integration of an exogenous sequence by homology-directed repair. In some embodiments, the cell comprises decreased transcription of a gene associated with the target sequence. In some embodiments, the cell comprises increased transcription of a gene associated with the target sequence. In some embodiments, the product is a mutated protein or product provided by a template.

In another aspect, the present disclosure provides a method of inserting a donor polynucleotide into a target polynucleotide in a cell, the method comprises introducing to the cell: one or more CRISPR-associated Tn7 or Tn7-like transposases or functional fragments thereof; one or more Type I-B Cas proteins; and a guide molecule capable of complexing with the Type I-B Cas protein and directing binding of the guide-Cas protein complex to a target polynucleotide.

In some embodiments, the donor polynucleotide: introduces one or more mutations to the target polynucleotide, corrects a premature stop codon in the target polynucleotide, disrupts a splicing site, restores a splicing site, or a combination thereof.

In some embodiments, the one or more mutations introduced by the donor polynucleotide comprises substitutions, deletions, insertions, or a combination thereof. In some embodiments, the one or more mutations causes a shift in an open reading frame on the target polynucleotide. In some embodiments, the donor polynucleotide is between 100 bases and 30 kb in length. In some embodiments, one or more of components (a), (b), and (c) is expressed from a nucleic acid operably linked to a regulatory sequence. In some embodiments, one or more of components (a), (b), and (c) is introduced in a particle. In some embodiments, the particle comprises a ribonucleoprotein (RNP). In some embodiments, the cell is a prokaryotic cell. In some embodiments, the cell is a eukaryotic cell. In some embodiments, the cell is a mammalian cell, a cell of a non-human primate, or a human cell. In some embodiments, the cell is a plant cell.

In some embodiments, insertion of the donor polynucleotide into the target polynucleotide in the cell results in a cell or population of cells comprising altered expression levels of one or more gene products; a cell or population of cells that produces and/or secrete an endogenous or non-endogenous biological product or chemical compound.

In some embodiments, the donor polynucleotide is inserted to a site no more than 80, no more 90, no more 100, no more 200, or no more 300 bp 3′ of a PAM on the target polynucleotide. In some embodiments, the donor polynucleotide is inserted to a site no more than 80, no more 90, no more 100, no more 200, or no more 300 bp 5′ of a PAM on the target polynucleotide. In some embodiments, the target polynucleotide is linear, circular, or genomic DNA.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a construct of an exemplary Type I-B CAST system from locus CP000117 of Anabaena variabilis ATCC 29413.

FIG. 2 shows plasmids for expressing the exemplary CAST system in Example 1.

FIG. 3 shows the maps of the plasmids in FIG. 2.

FIGS. 4-5 show the insertion of target sequences by the exemplary CAST system with different primer pairs.

FIG. 6 shows the sequences of the amplicons (by primer pairs a and b) recovered from gel bands from the tests shown in FIGS. 4-5.

FIG. 7 shows impacts of deletion of the components on the function of the exemplary CAST system.

FIG. 8 shows the sequences of the amplicons from the experiment shown in FIG. 7.

FIG. 9 shows purification of the proteins in CP000117: TnsA, TnsB, TniQ1, and TniQ2.

FIG. 10 shows testing results for targeting 10 genomic loci of BL21DE3.

FIG. 11 shows characterization of an exemplary CAST system.

FIG. 12 shows “N”AT-PAM tiling in glmS locus.

FIG. 13 shows the in vitro homing of an exemplary CAST system.

FIG. 14 shows the homing of an exemplary CAST system in 293T cells.

FIG. 15 shows evaluation of various conditions and their effects on the function of an exemplary CAST system.

FIG. 16 shows effects of various tags on the function of an exemplary CAST system.

FIG. 17 shows the expression of components of an exemplary CAST system in 293 cells.

FIG. 18 shows the plasmids used for testing the expression of the CAST components in FIG. 17.

FIGS. 19-21 show insertion of donor polynucleotides at target sites in mammalian cells by an exemplary CAST.

FIG. 22 shows characterization of the insertions in FIGS. 19-21 by sequencing.

FIG. 23 shows different functions of exemplary TniQ proteins.

FIG. 24 shows characterization of TnsB in an exemplary CAST system.

FIG. 25 PAM screening for an exemplary CAST system.

FIG. 26 shows minimal binding sites for an exemplary TnsB.

FIG. 27 shows consensus sequences of binding sites for an exemplary TnsB.

FIG. 28 shows various cargo sizes tested for an exemplary CAST system.

FIG. 29 shows results from evaluation of the insertion site specificity.

FIG. 30 shows the results of plasmid targeting and LE junction of an exemplary CAST system.

FIGS. 31-32. FIG. 31 shows the analysis of plasmids used in screening PAM of exemplary CAST systems. FIG. 32 shows the PAM screening results.

FIG. 33 shows analysis of competitive and/or cooperative effects of TnsD1 and TnsD2 on homing and CAST functions.

FIG. 34 shows testing of subcellular localization of plasmid targeting of an exemplary CAST-I-B system.

FIG. 35 shows exemplary Type I-B CAST systems.

FIG. 36 shows locus of T24 CAST and constructs for adapting the T24 CAST system in mammalian cells.

FIG. 37 shows expression of the components of an exemplary T24 CAST system in mammalian cells.

FIG. 38 shows cellular localization of NLS-tagged components of an exemplary T24-CAST system.

FIG. 39 shows results of GFP spiked-in transfection.

FIG. 40 shows experiment schemes for analyzing Tn7/CAST plasmid targeting in 293FT cells.

FIG. 41 shows plasmid targeting of an exemplary T24-Tn7 system in 293FT cells.

FIG. 42 shows plasmid targeting of an exemplary T24-CAST system in 293FT cells.

FIG. 43 shows analysis of individual components in an exemplary T24-CAST.

FIG. 44 shows effects of ratios between proteins and guide molecules on insertion activity.

FIG. 45 shows additional exemplary Type I-B CAST systems.

FIG. 46 shows exemplary methods for analyzing Type I-B CAST systems in E coli.

FIG. 47 shows exemplary methods for analyzing Type I-B CAST systems in mammalian cells.

FIG. 48 shows three exemplary Type I-B CAST systems tested work in mammalian plasmid targeting.

FIG. 49 shows PAM screening for an exemplary Type I-B CAST system.

FIG. 50 shows mammalian PAM screening pipelines for exemplary Type I-B CAST systems.

FIG. 51 shows testing PAM libraries in 293FT cells.

FIG. 52 shows results from PAM screening for exemplary Type I-B CAST systems.

FIG. 53 shows a map of an exemplary Type I-B CAST (IB18) locus.

FIG. 54 shows a map of an exemplary Type I-B CAST (IB28) locus.

FIG. 55 shows exemplary CAST systems comprising Type IB Cas proteins.

FIG. 56 constructs expressing exemplary CAST (T24) attached with various NLS.

FIG. 57 shows subcellular localization of components of T24 attached with v1 NLS

FIG. 58 shows subcellular localization of components of T24 attached with other versions of NLS.

FIG. 59 shows plasmids with the various NLS with T24 and their targeting activities.

FIG. 60 shows an exemplary experiment for testing mammalian genome targeting with t24.

FIG. 61 shows targeting of the plasmids in a 96-well format test with QE.

FIG. 62 shows PCR experiments performed to test the plasmids' effects on target genes.

FIG. 63 shows subcellular localization of Cascade components of IB28 CAST system.

FIG. 64 shows subcellular localization of Tns components of IB28 CAST system.

FIG. 65 shows subcellular localization of components of IB28 CAST system with various versions of NLSs.

FIG. 66 shows plasmids with the various NLS with IB28, and their targeting activities.

FIG. 67 shows subcellular localization of Cascade components of v.1 of NLS for IB18.

FIG. 68 shows subcellular localization of Tns components of v.1 of NLS for IB18.

FIG. 69 shows the locus of D320.

FIG. 70 shows the function of D320 in mammalian cells.

FIG. 71 shows the result from screening of PAM sequences.

FIG. 72 shows a map of IB20_pCMV-Cas5-NLS-HA and FIG. 73 shows the expression cassette.

FIG. 74 shows a map of IB20_pCMV-Cas6-NLS-HA and FIG. 75 shows the expression cassette.

FIG. 76 shows a map of IB20_pCMV-Cas7-NLS-HA and FIG. 77 shows the expression cassette.

FIG. 78 shows a map of IB20_pCMV-Cas8-NLS-HA and FIG. 79 shows the expression cassette.

FIG. 80 shows a map of IB20_pCMV-HA-NLS-Cas5 and FIG. 81 shows the expression cassette.

FIG. 82 shows a map of IB20_pCMV-HA-NLS-Cas6 and FIG. 83 shows the expression cassette.

FIG. 84 shows a map of IB20_pCMV-HA-NLS-Cas7 and FIG. 85 shows the expression cassette.

FIG. 86 shows a map of IB20_pCMV-HA-NLS-Cas8 and FIG. 87 shows the expression cassette.

FIG. 88 shows a map of IB20pCMV-HA-NLS-TniQ1 and FIG. 89 shows the expression cassette.

FIG. 90 shows a map of IB20pCMV-HA-NLS-TniQ2 and FIG. 91 shows the expression cassette.

FIG. 92 shows a map of IB20_pCMV-HA-NLS-TnsA and FIG. 93 shows the expression cassette.

FIG. 94 shows a map of IB20_pCMV-HA-NLS-TnsC and FIG. 95 shows the expression cassette.

FIG. 96 shows a map of IB20_pCMV-TniQ1-NLS-HA and FIG. 97 shows the expression cassette.

FIG. 98 shows a map of IB20_pCMV-TniQ2-NLS-HA and FIG. 99 shows the expression cassette.

FIG. 100 shows a map of IB20_pCMV-TnsA-NLS-HA and FIG. 101 shows the expression cassette.

FIG. 102 shows a map of IB20_pCMV-TnsC-NLS-HA and FIG. 103 shows the expression cassette.

FIG. 104 shows the map of pDonor_IB20-CmR.

FIG. 105 shows the map of pU6-IB20_CRISPR_PAMcont(−).

FIG. 106 shows the map of pU6-IB20_CRISPR PAMlib(+).

FIG. 107 shows example type I-b CAST T24 characterization in bacteria.

FIG. 108 shows T24 CAST function in targeting plasmids in mammalian cells

FIG. 109 shows screening of PAM of T24 CAST.

FIG. 110 shows PAM screening for additional CAST orthologs.

FIG. 111 shows IB20 locus and activity.

FIG. 112 shows example type I-b CAST loci.

FIGS. 113A-113B. NLSs were fused to the components of example CAST-1b systems and the subcellular localization of the components were tested by immunofluorescence.

FIGS. 114A-114B. Copy numbers of the plasmids needed for the T24 system to be active.

FIG. 115. Targeting LINE1-ORF2 by each of the four example CAST systems with 24 guide molecules.

FIG. 116. The CAST systems were active in targeting both super coiled and linear DNA target.

FIG. 117. Different sizes of the target sequences were tested, including linear targets comprising up to 300 bp upstream (5′ of PAM) and linear targets comprising up to 348 bp downstream (3′ of PAM).

FIG. 118. The minimal fragment size for CAST-1b function in 293FT cells.

FIGS. 119-120. Nucleosome accessibility of the Cas proteins in the Type I-b CAST systems was tested by transcription activation.

FIGS. 121-122. Individual Cas proteins in IB20 was fused with p300 and the transcription activation was tested.

FIG. 123 shows an example chimeric CAST system.

FIG. 124 shows the activities of example chimeric CAST systems in plasmid targeting in mammalian cells.

FIG. 125 shows the insertion positions example chimeric CAST systems.

FIGS. 126-127. The TnsD1 from both orthologs had an impact of the activity of the chimeric CAST.

FIGS. 128-130. Activities of the CAST systems with new NLSs in plasmid targeting.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides engineered systems and methods for inserting a polynucleotide to a desired position in a target nucleic acid. In general, the systems comprise one or more transposases or functional fragments thereof, and one or more components of a sequence-specific nucleotide binding system, e.g., a Cas protein and a guide molecule.

In some embodiments, the present disclosure provides engineered systems comprising one or more CRISPR-associated Tn7 transposases (e.g., TnsA, TnsB, TnsC, and/or TniQ; or TnsA, TnsB, TnsC, and/or TnsD) and one or more Type I-B Cas proteins. The systems may further comprise a guide molecule capable of complexing with the Cas protein and directing binding of the guide-Cas protein complex to a target polynucleotide. The present disclosure also includes polynucleotides encoding components of the nucleic acid targeting systems, and vector systems comprising one or more vectors comprising said polynucleotide. Further provided herein also includes cells, tissues, organs, and organisms comprising the systems or generated using the systems.

### Systems and Compositions

In one aspect, the present disclosure includes systems that comprise one or more transposases and nucleotide-binding molecules (e.g., nucleotide-binding proteins). The nucleotide binding proteins may be sequence-specific. The system may further comprise one or more transposon components. In some embodiments, the systems described herein may comprise a transposase(s) that is associated with, linked to, bound to, or otherwise capable of forming a complex with a sequence-specific nucleotide-binding system. In certain example embodiments, the one or more transposases and the sequence-specific nucleotide-binding system are associated by co-regulation or expression. In other example embodiments, the transposase(s) and sequence-specific nucleotide binding system are associated by the ability of the sequence-specific nucleotide-binding domain to direct or recruit the transposase(s) to an insertion site where the transposase(s) direct insertion of a donor polynucleotide into a target polynucleotide sequence. A sequence-specific nucleotide-binding system may be a sequence-specific DNA-binding protein, or functional fragment thereof, and/or sequence-specific RNA-binding protein or functional fragment thereof. In some embodiments, a sequence-specific nucleotide-binding component may be a CRISPR-Cas system, a transcription activator-like effector nuclease, a Zn finger nuclease, a meganuclease, a functional fragment, a variant thereof, of any combination thereof. Accordingly, the system may also be considered to comprise a nucleotide binding component and a transposase. For ease of reference, further example embodiments will be discussed in the context of example Cas-associated transposase systems.

In some examples, the system may be an engineered system, the system comprising one or more CRISPR-associated Tn7 transposases or functional fragments thereof; one or more Type I-B Cas proteins; and a guide molecule capable of complexing with the Cas protein and directing binding of the guide-Cas protein complex to a target polynucleotide.

In certain examples, the system may comprise polynucleotides with sequences encoding the one or more transposases, Cas proteins, and guide sequences.

The system may be used to target various types of target polynucleotides. In some examples, the target polynucleotides may be linear DNA. In some examples, the target polynucleotides may be circular (e.g., supercoiled) DNA such as plasmids. In some examples, the target polynucleotides may genomic DNA. The target polynucleotide may be a polynucleotide in a eukaryotic cell. For example, the target polynucleotide may be a polynucleotide in the genome of a eukaryotic cell. The genome may be the nuclear genome, mitochondrial genome, or chloroplast genome.

When a term refers to a protein, e.g., Cas protein, transposase, etc., the term encompasses both the full-length of the protein as well as a functional fragment of the protein. The term “functional fragment” means that the sequence of the polypeptide may include less amino-acid than the original sequence but still enough amino-acids to confer the enzymatic activity of the original sequence of reference. It is well known in the art that a polypeptide can be modified by substitution, insertion, deletion and/or addition of one or more amino-acids while retaining its enzymatic activity. For example, substitutions of one amino-acid at a given position by chemically equivalent amino-acids that do not affect the functional properties of a protein are common.

### Transposons and Transposases

The systems herein may comprise one or more components of a transposon and/or one or more transposases. The transposases in the systems herein may be CRISPR-associated transposases (also used interchangeably with Cas-associated transposases, CRISPR-associated transposase proteins herein) or functional fragments thereof. CRISPR-associated transposases may include any transposases that can be directed to or recruited to a region of a target polynucleotide by sequence-specific binding of a CRISPR-Cas complex. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g. by a linker) to one or more components in a CRISPR-Cas system, e.g., Cas protein, guide molecule etc.).

The term “transposon”, as used herein, refers to a polynucleotide (or nucleic acid segment), which may be recognized by a transposase or an integrase enzyme and which is a component of a functional nucleic acid-protein complex (e.g., a transpososome, or transposon complex) capable of transposition. Transposons employ a variety of regulatory mechanisms to maintain transposition at a low frequency and sometimes coordinate transposition with various cell processes. Some prokaryotic transposons can also mobilize functions that benefit the host or otherwise help maintain the element.

The term “transposase” as used herein refers to an enzyme, which is a component of a functional nucleic acid-protein complex capable of transposition and which mediates transposition. The transposase may comprise a single protein or comprise multiple protein subunits. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

In some embodiments, the system comprises one or more Tn7 transposases. In some embodiments, three transposon-encoded proteins form the core transposition machinery of Tn7: a heteromeric transposase (TnsA and TnsB) and a regulator protein (TnsC). In addition to the core TnsABC transposition proteins, Tn7 elements encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ, a protein found in other types of bacterial transposons. TniQ has been shown to target transposition into resolution sites of plasmids. As used herein, a TniQ transposase may be a TnsD transposase. In some examples, the Tn7 comprises a transposase that has the activities of typical TnsA and TnsB. In some examples, the transposase is not a fusion protein of typical TnsA and TnsB. An example of the transposase is TnsA in IB20.

Examples of Tn7 transposases include TnsA, TnsB, TnsC, TniQ, TnsD, and TnsE. In some embodiments, the system comprises TnsA, TnsB, TnsC, and/or TniQ. In some embodiments, the system comprises TnsA, TnsB, TnsC, and/or TnsD (e.g., TnsD2). In some examples, the system comprises TnsA, TnsB, TnsC, and TniQ (e.g., TniQ2). In some examples, the system comprises TnsA, TnsB, TnsC, and TnsD (e.g., TnsD2). In some examples, the system comprises two or more TnsA. In some examples, the system comprises two or more TnsA (e.g., 2 TnsA). In some examples, the system comprises two or more TnsB (e.g., 2 TnsB). In some examples, the system comprises two or more TnsC (e.g., 2 TnsC). In some examples, the system comprises two or more TnsD (e.g., 2 TnsDs). In some examples, the system comprises two or more TniQ (e.g., 2 TniQs). The TniQ or TnsD may comprise a DNA-binding domain. The DNA-binding domain may be at the C terminus of the TniQ or TnsD. In some cases, the DNA-binding domain may be at the N terminus, or between the N- and C-termini of the TniQ or TnsD. In some cases, the system comprises TnsA, TnsB, TnsC, and only one TniQ or TnsD, e.g., such TniQ or TnsD may comprise a DNA-binding domain. In a particular example, the system comprises TnsA, TnsB, TnsC, and TnsD1. In another example, the system comprises TnsA, TnsB, TnsC, and TnsD2. In another example, the system comprises TnsA, TnsB, TnsC, TnsD1, and TnsD2. Two or more of the components in the system may be comprised in a single protein (e.g., fusion protein). For example, TnsA and TnsB may be comprised in a single protein.

In some embodiments, the system comprises one or more polynucleotides encoding one or more of the Tn7 transposases. In some examples, the system comprises one or more polynucleotides encoding TnsA. In some examples, the system comprises one or more polynucleotides encoding TnsB. In some examples, the system comprises one or more polynucleotides encoding TnsC. In some examples, the system comprises one or more polynucleotides encoding TnsD. In some examples, the system comprises one or more polynucleotides encoding TnsE. In some examples, the system comprises one or more polynucleotides encoding TniQ. The system may comprise two or more polynucleotides encoding the same type of transposase. In one example, the system may comprise two or more polynucleotides encoding TnsA (the same or different TnsA). In one example, the system may comprise two or more polynucleotides encoding TnsB (the same or different TnsB). In one example, the system may comprise two or more polynucleotides encoding TnsC (the same or different TnsC). In one example, the system may comprise two or more polynucleotides encoding TnsD (the same or different TnsD). In one example, the system may comprise two or more polynucleotides encoding TnsE (the same or different TnsE). In one example, the system may comprise two or more polynucleotides encoding TniQ (the same or different TniQ).

As used herein, a right end sequence element or a left end sequence element are made in reference to an example Tn7 transposon. The general structure of the left end (LE) and right end (RE) sequence elements of canonical Tn7 is established. Tn7 ends comprise a series of 22-bp TnsB-binding sites. Flanking the most distal TnsB-binding sites is an 8-bp terminal sequence ending with 5′-TGT-3′/3′-ACA-5′. The right end of Tn7 contains four overlapping TnsB-binding sites in the ˜90-bp right end element. The left end contains three TnsB-binding sites dispersed in the ˜150-bp left end of the element. The number and distribution of TnsB-binding sites can vary among Tn7-like elements. End sequences of Tn7-related elements can be determined by identifying the directly repeated 5-bp target site duplication, the terminal 8-bp sequence, and 22-bp TnsB-binding sites (Peters J E et al., 2017). Example Tn7 elements, including right end sequence element and left end sequence element include those described in Parks A R, Plasmid, 2009 January; 61(1):1-14.

As used herein, Tn7 transposons and transposases include Tn7-like transposons and transposases.

### Donor Polynucleotides

The systems may comprise one or more donor polynucleotides (e.g., for insertion into the target polynucleotide). A donor polynucleotide may be an equivalent of a transposable element that can be inserted or integrated to a target site. For example, the donor polynucleotide may comprise a polynucleotide to be inserted, a left element sequence, and a right element sequence. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc.

In some embodiments, the donor polynucleotide is linear. In some embodiments, the donor polynucleotide is circular. In some examples, the donor polynucleotide has a single strand break (a nick). In some cases, the single strand break is on or close to the 3′ end of the donor polynucleotide. In some cases, the single strand break is on or close to the 5′ end of the donor polynucleotide.

In some embodiments, the donor polynucleotide is inserted to a site no more than 80, no more 90, no more 100, no more 200, or no more 300 bp 3′ of the PAM. In some examples, the donor polynucleotide is inserted to a site that is 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 bp 3′ of the PAM.

In some embodiments, the donor polynucleotide is inserted to a site no more than 80, no more 90, no more 100, no more 200, or no more 300 bp 5′ of the PAM. In some examples, the donor polynucleotide is inserted to a site that is 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 bp 5′ of the PAM.

A target polynucleotide may comprise a protospacer adjacent motif (PAM) sequence. An example of the PAM sequence is AT.

The donor polynucleotides may be inserted to the upstream or downstream of the PAM sequence of a target polynucleotide. For CRISPR-associated transposases, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a the corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 10 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

### CRISPR-Cas Systems

The systems herein may comprise one or more components of a CRISPR-Cas system. The one or more components of the CRISPR-Cas system may serve as the nucleotide-binding component in the systems. The nucleotide-binding molecule may be a Cas protein (used interchangeably with CRISPR protein, CRISPR enzyme, Cas effector, CRISPR-Cas protein, CRISPR-Cas enzyme), a fragment thereof, or a mutated form thereof. The Cas protein may have reduced or no nuclease activity. For example, the Cas protein may be an inactive or dead Cas protein (dCas). The dead Cas protein may comprise one or more mutations or truncations. In some examples, the DNA binding domain comprises one or more Class I (e.g., Type I, Type III, Type VI) or Class 2 (e.g., Type II, Type V, or Type VI) CRISPR-Cas proteins. In certain embodiments, the sequence-specific nucleotide binding domains directs a transposon to a target site comprising a target sequence and the transposase directs insertion of a donor polynucleotide sequence at the target site. In certain example embodiments, the transposon component includes, associates with, or forms a complex with a CRISPR-Cas complex. In one example embodiment, the CRISPR-Cas component directs the transposon component and/or transposase(s) to a target insertion site where the transposon component directs insertion of the donor polynucleotide into a target nucleic acid sequence.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

The CRISPR-Cas systems herein may comprise a Cas protein and a guide molecule. In some embodiments, the system comprises one or more Cas proteins. The Cas proteins may be Type 1 Cas proteins, e.g., Cas proteins of Type I CRISPR-Cas systems.

In certain example embodiments, the CRISPR-Cas system is a Class 1 CRISPR-Cas system, e.g., a Class 1 Type I CRISPR-Cas system. In some cases, a Class I CRISPR-Cas system comprises Cascade (a multimeric complex comprising three, four, or five proteins that processes crRNA arrays), Cas3 (a protein with nuclease, helicase, and exonuclease activity that is responsible for degradation of the target DNA), and crRNA (stabilizes Cascade complex and directs Cascade and Cas3 to DNA target). A Class 1 CRISPR-Cas system may be of a subtype, e.g., Type I-A, Type I-B, Type I-C, Type I-D, Type I-E, Type I-F, Type I-U, Type III-A, Type-III-B, Type-III-C, Type-III-D, or Type-IV CRISPR-Cas system.

The Class 1 type I CRISPR Cas system may be used to catalyze RNA-guided integration of mobile genetic elements into a target nucleic acid (e.g., genomic DNA). For example, the systems herein may comprise a complex between Cascade and a transposon protein. At a given distance downstream of a target nucleic acid, a donor nucleic acid (e.g., DNA) may be inserted. The insertion may be in one of two possible orientations. The system may be used to integrate a nucleic acid sequence of desired length. In some examples, the type I CRISPR-Cas system is nuclease-deficient. In some examples, the type I CRISPR-Cas system is Type I-B CRISPR-Cas system.

A Class 1 type I-A CRISPR-Cas system may comprise Cas7 (Csa2), Cas8a1 (Csx13), Cas8a2 (Csx9), Cas5, Csa5, Cas6a, Cas3′ and/or a Cas3. A type I-B CRISPR-Cas system may comprise Cas6b, Cas8b (Csh1), Cas7 (Csh2) and/or Cas5. A type I-C CRISPR-Cas system may comprise Cas5d, Cas8c (Csd1), and/or Cas7 (Csd2). A type I-D CRISPR-Cas system may comprise Cas10d (Csc3), Csc2, Csc1, and/or Cas6d. A type I-E CRISPR-Cas system may comprise Cse1 (CasA), Cse2 (CasB), Cas7 (CasC), Cas5 (CasD) and/or Cas6e (CasE). A type I-F CRISPR-Cas system may comprise Cys1, Cyst, Cas7 (Cys3) and/or Cas6f (Csy4). An example type I-F CRISPR-Cas system may include a DNA-targeting complex Cascade (also known as Csy complex) which is encoded by three genes: cash, cas7, and a natural cas8-cas5 fusion (hereafter referred to simply as cas8). The type I-F CRISPR-Cas system may further comprise a native CRISPR array, comprising four repeat and three spacer sequences, encodes distinct mature CRISPR RNAs (crRNAs), which we also refer to as guide RNAs.

In some examples, a Type I CRISPR-Cas system may comprise one or more: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, a nucleotide sequence encoding a Cas5 polypeptide, a nucleotide sequence encoding a Cas3′ polypeptide, and a nucleotide sequence encoding a Cas3″ polypeptide (Type I-B); (c) a nucleotide sequence encoding a Cas5d polypeptide, a nucleotide sequence encoding a Cas8c (Csd1) polypeptide, a nucleotide sequence encoding a Cas7 (Csd2) polypeptide and a nucleotide sequence encoding a Cas3 polypeptide (Type I-C); (d) a nucleotide sequence encoding a Cas10d (Csc3) polypeptide, a nucleotide sequence encoding a Csc2 polypeptide, a nucleotide sequence encoding a Csc1 polypeptide, a nucleotide sequence encoding a Cas6d polypeptide, and a nucleotide sequence encoding a Cas3 polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, a nucleotide sequence encoding a Cas6e (CasE) polypeptide, and a nucleotide sequence encoding a Cas3 polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide and a nucleotide sequence encoding a Cas6f polypeptide, and a nucleotide sequence encoding a Cas3 polypeptide (Type I-F). Accordingly, a type I Cas protein may be one or more of the Cas protein described herein.

In some embodiments, the system comprises one or more Type I-B Cas proteins. I The one or more Type I-B Cas proteins may comprise Cas6b. The one or more Type I-B Cas proteins may comprise Cas8b, e.g., Cas8b1, Cas8b2, and Cas8b3. In some examples, the one or more Type I-B Cas proteins comprises Cas8b3. In some examples, the Type 1-B Cas protein may be one or more of Cas5, Cas6, Cas7, and Cas8. In some examples, the system comprises Cas 5. In some examples, the system comprises Cas 6. In some examples, the system comprises Cas 7. In some examples, the system comprises Cas 5 and Cas6. In some examples, the system comprises Cas 5 and Cas7. In some examples, the system comprises Cas 5 and Cas 8. In some examples, the system comprises Cas 6 and Cas 7. In some examples, the system comprises Cas 6 and Cas 8. In some examples, the system comprises Cas 7 and Cas 8. In some examples, the system comprises Cas 5, Cas6, and Cas7. In some examples, the system comprises Cas 5, Cas6, and Cas8. In some examples, the system comprises Cas 5, Cas7 and Cas8. In some examples, the system comprises Cas 6, Cas7, and Cas8. In some examples, the system comprises Cas 5, Cas6, Cas7, and Cas8.

In some examples, the system comprises a polynucleotide encoding Cas5. In some examples, the system comprises a polynucleotide encoding Cas6. In some examples, the system comprises a polynucleotide encoding Cas7. In some examples, the system comprises a polynucleotide encoding Cas 5 and a polynucleotide encoding Cas6. In some examples, the system comprises a polynucleotide encoding Cas5 and a polynucleotide encoding Cas7. In some examples, the system comprises a polynucleotide encoding Cas 5 and a polynucleotide encoding Cas8. In some examples, the system comprises a polynucleotide encoding Cas6 and a polynucleotide encoding Cas7. In some examples, the system comprises a polynucleotide encoding Cas6 and a polynucleotide encoding Cas8. In some examples, the system comprises a polynucleotide encoding Cas7 and a polynucleotide encoding Cas 8. In some examples, the system comprises a polynucleotide encoding Cas 5, a polynucleotide encoding Cas6, and a polynucleotide encoding Cas7. In some examples, the system comprises a polynucleotide encoding Cas 5, a polynucleotide encoding Cas6, and a polynucleotide encoding Cas8. In some examples, the system comprises a polynucleotide encoding Cas 5, a polynucleotide encoding Cas7 and a polynucleotide encoding Cas8. In some examples, the system comprises a polynucleotide encoding Cas 6, a polynucleotide encoding Cas7, and a polynucleotide encoding Cas8. In some examples, the system comprises a polynucleotide encoding Cas 5, a polynucleotide encoding Cas6, a polynucleotide encoding Cas7, and a polynucleotide encoding Cas8. The Cas proteins herein (e.g., Cas5, Cas6, Cas7, Cas 8) includes the wild type transposases, variants thereof, and functional fragments thereof. In some examples, the system comprises a first polynucleotide encoding a first Cas6 and a second polynucleotide encoding a second Cas6.

Examples of type I CRISPR components include those described in Makarova et al., Annotation and Classification of CRISPR-Cas Systems, Methods Mol Biol. 2015; 1311: 47-75.

In some examples, the Cas protein may be nuclease-deficient. A nuclease-deficient nuclease may have no nuclease activity. A nuclease-deficient nuclease may have nickase activity.

In some cases, the Cas protein may be orthologues or homologues of the above mentioned Cas proteins. The terms “ortholog” and “homolog” are well known in the art. By means of further guidance, a “homolog” of a protein as used herein is a protein of the same species which performs the same or a similar function as the protein it is a homologue of. Homologous proteins may but need not be structurally related, or are only partially structurally related. An “ortholog” of a protein as used herein is a protein of a different species which performs the same or a similar function as the protein it is an orthologue of. Orthologous proteins may but need not be structurally related, or are only partially structurally related.

In some examples, the one or more CRISPR-associated Tn7 transposases and/or the one or more Type I-B Cas proteins are from or originated from Anabaena variabilis.

In some cases, the Cas protein lacks nuclease activity. Such Cas protein may be a naturally existing Cas protein that does not have nuclease activity or the Cas protein may be an engineered Cas protein with mutations or truncations that reduce or eliminate nuclease activity.

In certain embodiments, the present disclosure includes a transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to International Patent Publication No. WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

The guide RNA(s) encoding sequences and/or Cas encoding sequences can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

### Guide Molecules

The system herein may comprise one or more guide molecules. In some cases the system comprises one guide molecule. In certain cases, the system comprises a plurality of guide molecules. The guide molecule(s) may direct or may be capable of directing the binding of a guide-Cas protein complex to one or more target polynucleotides. For example, the system herein may be used for inserting a donor polynucleotide to one or more desired target sites with the direction of the guide molecule(s).

The guide molecule(s) may be component(s) of the CRISPR-Cas system herein. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, a guide molecule is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, it is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will often be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In some embodiments, it is of interest to reduce the susceptibility of the guide molecule to RNA cleavage, such as cleavage by a CRISPR system that cleaves RNA. Accordingly, in particular embodiments, the guide molecule is adjusted to avoid cleavage by a CRISPR system or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to a Cas effector. In an embodiment of the invention, deoxyribonucleotides and/or nucleotide analogs are incorporated in engineered guide structures, such as, without limitation, stem-loop regions, and the seed region. In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-0-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine, peptides, nuclear localization sequence (NLS), peptide nucleic acid (PNA), polyethylene glycol (PEG), triethylene glycol, or tetraethyleneglycol (TEG). In certain embodiment, the chemical moiety is conjugated to the guide by a linker, such as an alkyl chain. In certain embodiments, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235). In some embodiments, more than 60 or 70 nucleotides of the guide are chemically modified. In some embodiments, this modification comprises replacement of nucleotides with 2′-O-methyl or 2′-fluoro nucleotide analogs or phosphorothioate (PS) modification of phosphodiester bonds. In some embodiments, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In a particular embodiment, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see Yin et al., Nat. Biotech. (2018), 35(12): 1179-1187). Such chemical modifications may be guided by knowledge of the structure of the CRISPR complex, including knowledge of the limited number of nuclease and RNA 2′-OH interactions (see Yin et al., Nat. Biotech. (2018), 35(12): 1179-1187). In some embodiments, one or more guide RNA nucleotides may be replaced with DNA nucleotides. In some embodiments, up to 2, 4, 6, 8, 10, or 12 RNA nucleotides of the 5′-end tail/seed guide region are replaced with DNA nucleotides. In certain embodiments, the majority of guide RNA nucleotides at the 3′ end are replaced with DNA nucleotides. In particular embodiments, 16 guide RNA nucleotides at the 3′ end are replaced with DNA nucleotides. In particular embodiments, 8 guide RNA nucleotides of the 5′-end tail/seed region and 16 RNA nucleotides at the 3′ end are replaced with DNA nucleotides. In particular embodiments, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) or downstream (i.e. 3′) from the guide sequence. In a particular embodiment, the seed sequence (i.e. the sequence essential critical for recognition and/or hybridization to the sequence at the target locus) of the guide sequence is approximately within the first 10 nucleotides of the guide sequence.

In a particular embodiment, the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments, the guide molecule comprises or consists of the guide sequence linked to all or part of the natural direct repeat sequence. A CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments, the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments, the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments, these are located at the end of the stem, adjacent to the loop of the stemloop.

In particular embodiments, the susceptibility of the guide molecule to RNases or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA, may be selected to target any target nucleic acid sequence. The target sequence may be mRNA.

In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592.

In particular embodiments, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends a guide molecule that responds to normal or pathological physiological conditions, including without limitation pH, hypoxia, O2 concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the CRISPR-Cas system or complex function. The invention can involve applying the chemical source or energy so as to have the guide function and the CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/r52), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the CRISPR-Cas complex or system, and the binding will induce the change of sub-cellular localization of the polypeptide, leading to the entire polypeptide entering the nucleus of cells. Once inside the nucleus, the guide protein and the other components of the CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably, the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu·s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### Additional CRISPR-Cas Development and Use Considerations

The present invention may be further illustrated and extended based on aspects of CRISPR-Cas9 development and use as set forth in the following articles and particularly as relates to delivery of a CRISPR protein complex and uses of an RNA guided endonuclease in cells and organisms:
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014 (2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1. [Epub ahead of print].
Gao et al, “Engineered Cpf1 Enzymes with Altered PAM Specificities,” bioRxiv 091611; doi: http://dx.doi.org/10.1101/091611 (Dec. 4, 2016)
   each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and gRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors we developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

The methods and tools provided herein are exemplified for certain Cas effectors. Further nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-027323 A1 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 A1 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 A1 (U.S. application Ser. No. 14/183,429); US 2015-0184139 A1 (U.S. application Ser. No. 14/324,960); Ser. No. 14/054,414 European Patent Applications EP 2771468 (EP13818570.7), EP 2764103 (EP13824232.6), and EP 2784162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014.

Mention is also made of U.S. Provisional Application No. 62/180,709, filed 17 Jun. 2015, PROTECTED GUIDE RNAS (PGRNAS); U.S. Provisional Application No. 62/091,455, filed 12 Dec. 2014, PROTECTED GUIDE RNAS (PGRNAS); U.S. Provisional Application No. 62/096,708, filed 24 Dec. 2014, PROTECTED GUIDE RNAS (PGRNAS); US Provisional Application Nos. 62/091,462, filed 12 Dec. 2014, 62/096,324, filed 23 Dec. 2014, 62/180,681, filed 17 Jun. 2015, and 62/237,496, filed 5 Oct. 2015, DEAD GUIDES FOR CRISPR TRANSCRIPTION FACTORS; US Provisional Application Nos. 62/091,456, filed 12 Dec. 2014 and 62/180,692, filed 17 Jun. 2015, ESCORTED AND FUNCTIONALIZED GUIDES FOR CRISPR-CAS SYSTEMS; U.S. Provisional Application No. 62/091,461, filed 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. Provisional Application No. 62/094,903, filed 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. Provisional Application No. 62/096,761, filed 24 Dec. 2014, ENGINEERING OF SYSTEMS, METHODS AND OPTIMIZED ENZYME AND GUIDE SCAFFOLDS FOR SEQUENCE MANIPULATION; U.S. Provisional Application No. 62/098,059, filed 30 Dec. 2014, 62/181,641, filed 18 Jun. 2015, and 62/181,667, filed 18 Jun. 2015, RNA-TARGETING SYSTEM; U.S. Provisional Application No. 62/096,656, filed 24 Dec. 2014 and 62/181,151, filed 17 Jun. 2015, CRISPR HAVING OR ASSOCIATED WITH DESTABILIZATION DOMAINS; U.S. Provisional Application No. 62/096,697, filed 24 Dec. 2014, CRISPR HAVING OR ASSOCIATED WITH AAV; U.S. Provisional Application 62/098,158, filed 30 Dec. 2014, ENGINEERED CRISPR COMPLEX INSERTIONAL TARGETING SYSTEMS; U.S. Provisional Application No. 62/151,052, filed 22 Apr. 2015, CELLULAR TARGETING FOR EXTRACELLULAR EXOSOMAL REPORTING; U.S. Provisional Application No. 62/054,490, filed 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. Provisional Application No. 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. Provisional Application No. 62/055,484, filed 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. Provisional Application No. 62/087,537, filed 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. Provisional Application No. 62/054,651, filed 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. Provisional Application No. 62/067,886, filed 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; US Provisional Application Nos. 62/054,675, filed 24 Sep. 2014 and 62/181,002, filed 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. Provisional Application 62/054,528, filed 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. Provisional Application No. 62/055,454, filed 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. Provisional Application No. 62/055,460, filed 25 Sep. 2014, MULTIFUNCTIONAL-CRISPR COMPLEXES AND/OR OPTIMIZED ENZYME LINKED FUNCTIONAL-CRISPR COMPLEXES; U.S. Provisional Application No. 62/087,475, filed 4 Dec. 2014 and 62/181,690, filed 18 Jun. 2015, FUNCTIONAL SCREENING WITH OPTIMIZED FUNCTIONAL CRISPR-CAS SYSTEMS; U.S. Provisional Application 62/055,487, filed 25 Sep. 2014, FUNCTIONAL SCREENING WITH OPTIMIZED FUNCTIONAL CRISPR-CAS SYSTEMS; U.S. Provisional Application No. 62/087,546, filed 4 Dec. 2014 and 62/181,687, filed 18 Jun. 2015, MULTIFUNCTIONAL CRISPR COMPLEXES AND/OR OPTIMIZED ENZYME LINKED FUNCTIONAL-CRISPR COMPLEXES; and U.S. Provisional Application 62/098,285, filed 30 Dec. 2014, CRISPR MEDIATED IN VIVO MODELING AND GENETIC SCREENING OF TUMOR GROWTH AND METASTASIS.

Mention is made of US Provisional Application Nos. 62/181,659, filed 18 Jun. 2015 and 62/207,318, filed 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of US Provisional Applications Nos. 62/181,663, filed 18 Jun. 2015 and 62/245,264, filed 22 Oct. 2015, NOVEL CRISPR ENZYMES AND SYSTEMS, US Provisional Application Nos. 62/181,675, filed 18 Jun. 2015, 62/285,349, filed 22 Oct. 2015, 62/296,522, filed 17 Feb. 2016, and 62/320,231, filed 8 Apr. 2016, NOVEL CRISPR ENZYMES AND SYSTEMS, U.S. Provisional Application No. 62/232,067, filed 24 Sep. 2015, U.S. application Ser. No. 14/975,085, filed 18 Dec. 2015, European Application No. 16150428.7, U.S. Provisional Application 62/205,733, filed 16 Aug. 2015, U.S. Provisional Application 62/201,542, filed 5 Aug. 2015, U.S. Provisional Application No. 62/193,507, filed 16 Jul. 2015, and U.S. Provisional Application No. 62/181,739, filed 18 Jun. 2015, each entitled NOVEL CRISPR ENZYMES AND SYSTEMS, and of U.S. Provisional Application No. 62/245,270, filed 22 Oct. 2015, NOVEL CRISPR ENZYMES AND SYSTEMS. Mention is also made of U.S. Provisional Application No. 61/939,256, filed 12 Feb. 2014, and WO 2015/089473 (PCT/US2014/070152), filed 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of International Application No. PCT/US2015/045504, filed 15 Aug. 2015, U.S. Provisional Application No. 62/180,699, filed 17 Jun. 2015, and U.S. Provisional Application No. 62/038,358, filed 17 Aug. 2014, each entitled GENOME EDITING USING CAS9 NICKASES.

In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, and of PCT application PCT/US14/70127, Attorney Reference 47627.99.2091 and BI-2013/101 entitled “DELIVERY, USE AND THERAPEUTIC APPLICATIONS OF THE CRISPR-CAS SYSTEMS AND COMPOSITIONS FOR GENOME EDITING” (claiming priority from one or more or all of US provisional patent applications: 61/915,176; 61/915,192; 61/915,215; 61/915,107, 61/915,145; 61/915,148; and 61/915,153 each filed Dec. 12, 2013) (“the Eye PCT”), incorporated herein by reference, with respect to a method of preparing an sgRNA-and-Cas protein containing particle comprising admixing a mixture comprising an sgRNA and Cas effector protein (and optionally HDR template) with a mixture comprising or consisting essentially of or consisting of surfactant, phospholipid, biodegradable polymer, lipoprotein and alcohol; and particles from such a process. For example, wherein the Cas protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C1-6 alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. Other example nucleotide-binding systems and proteins

### Other Exemplary Nucleotide-Binding Molecules and Systems

In certain embodiments, the system or composition herein comprises one or more transposases and one or more nucleotide-binding molecules that are not components of a CRISPR-Cas system. Examples of the other nucleotide-binding molecules may be components of transcription activator-like effector nuclease (TALEN), Zn finger nucleases, meganucleases, a functional fragment thereof, a variant thereof, of any combination thereof. In some examples, the systems comprise i) one or more components of a TALEN system, Zn finger nuclease system, or meganuclease system, and ii) one or more transposases described herein.

### TALE Systems

In some embodiment, the nucleotide-binding molecule in the systems may be a transcription activator-like effector nuclease, a functional fragment thereof, or a variant thereof. The present disclosure also includes nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, provided herein include isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X₁₋₁₁-(X₁₂X₁₃)-X₁₄₋₃₃ or ₃₄ or ₃₅)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

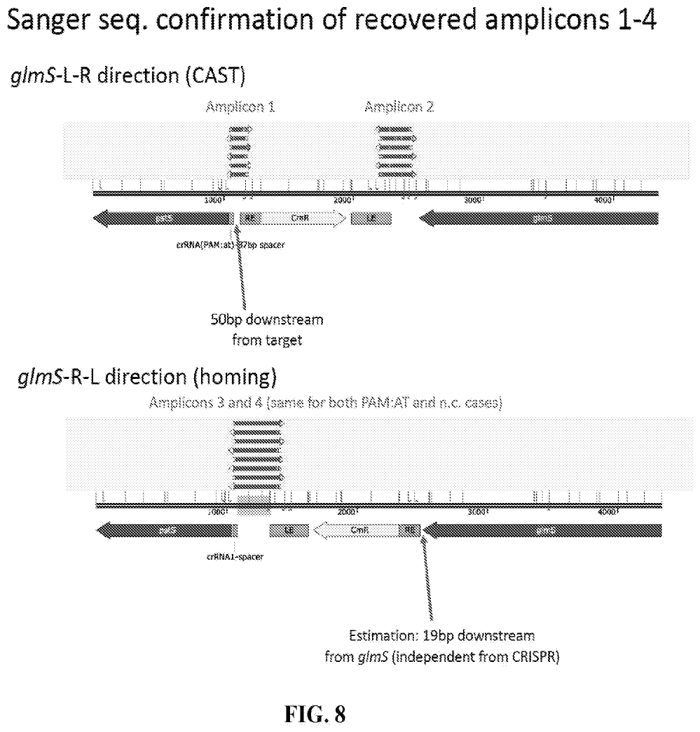

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 2)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

In some embodiment, the nucleotide-binding molecule of the systems may be a Zn-finger nuclease, a functional fragment thereof, or a variant thereof. The composition may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiment, the nucleotide-binding domain may be a meganuclease, a functional fragment thereof, or a variant thereof. The composition may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### Linkers

The transposase(s) and the Cas protein(s) may be associated via a linker. The term “linker” refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods herein include straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the transposase by a distance sufficient to ensure that each protein retains its required functional property. A peptide linker sequences may adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180.

For example, GlySer linkers GGS, GGGS (SEQ ID NO: 3) or GSG can be used. GGS, GSG, GGGS (SEQ ID NO: 3) or GGGGS (SEQ ID NO: 4) linkers can be used in repeats of 3 (such as (GGS)₃ (SEQ ID NO: 5), (GGGGS)₃ (SEQ ID NO: 6)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 4), (GGGGS)₂ (SEQ ID NO: 7), (GGGGS)₃ (SEQ ID NO: 6), (GGGGS)₄ (SEQ ID NO: 8), (GGGGS)₅ (SEQ ID NO: 9), (GGGGS)₆ (SEQ ID NO: 10), (GGGGS)₇ (SEQ ID NO: 11), (GGGGS)₈ (SEQ ID NO: 12), (GGGGS)₉ (SEQ ID NO: 13), (GGGGS)₁₀ (SEQ ID NO: 14), or (GGGGS)₁₁ (SEQ ID NO: 15).

In particular embodiments, linkers such as (GGGGS)₃ (SEQ ID NO: 6) are preferably used herein. (GGGGS)₆ (SEQ ID NO: 10), (GGGGS)₉ (SEQ ID NO: 13) or (GGGGS)₁₂ (SEQ ID NO: 16) may be used as alternatives. Other alternatives include (GGGGS)₁ (SEQ ID NO: 4), (GGGGS)₂ (SEQ ID NO: 7), (GGGGS)₄ (SEQ ID NO: 8), (GGGGS)₅ (SEQ ID NO: 9), (GGGGS)₇ (SEQ ID NO: 11), (GGGGS)₈ (SEQ ID NO: 12), (GGGGS)₁₀ (SEQ ID NO: 14), or (GGGGS)₁₁ (SEQ ID NO: 15). In yet a further embodiment, LEPGEKPYKCPECGKSFSQSGALTRHQRTHTR (SEQ ID NO: 17) is used as a linker. In yet an additional embodiment, the linker is an XTEN linker. In particular embodiments, the Cas protein is linked to the deaminase protein or its catalytic domain by means of an LEPGEKPYKCPECGKSFSQSGALTRHQRTHTR (SEQ ID NO: 17) linker. In further particular embodiments, the Cas protein is linked C-terminally to the N-terminus of a deaminase protein or its catalytic domain by means of an LEPGEKPYKCPECGKSFSQSGALTRHQRTHTR (SEQ ID NO: 17) linker. In addition, N- and C-terminal NLSs can also function as linker (e.g., PKKKRKVEASSPKKRKVEAS (SEQ ID NO: 18)).

Examples of linkers are shown in the Table below.

TABLE 1

| | |
|---|---|
| GGS | GGTGGTAGT (SEQ ID NO: 19) |
| GGSx3 (9) | GGTGGTAGTGGAGGGAGCGGCGGTTCA (SEQ ID NO: 20) |
| (SEQ ID NO: 5) | |
| GGSx7 (21) | ggtggaggaggctctggtggaggcggtagcggaggcggagggtcgGGTGGTAGTGGAGGG |
| SEQ ID NO: 21) | AGCGGCGGTTCA (SEQ ID NO: 22) |
| XTEN | TCGGGATCTGAGACGCCTGGGACCTCGGAATCGGCTACGCCCGAA |
| | AGT(SEQ ID NO: 23) |
| Z-EGFR_Short | Gtggataacaaatttaacaaagaaatgtgggcggcgtgggaagaaattcgtaacctgccgaacctgaacggc |
| | tggcagatgaccgcgtttattgcgagcctggtggatgatccgagccagagcgcgaacctgctggcggaagcg |
| | aaaaaactgaacgatgcgcaggcgccgaaaaccggcggtggttctggt (SEQ ID NO: 24) |
| GSAT | Ggtggttctgccggtggctccggttctggctccagcggtggcagctctggtgcgtccggcacgggtactgcg |
| | ggtggcactggcagcggttccggtactggctctggc (SEQ ID NO: 25) |

Linkers may be used between the guide RNAs and the functional domain (activator or repressor), or between the Cas protein and the transposase(s). The linkers may be used to engineer appropriate amounts of “mechanical flexibility”.

In certain embodiments, the one or more functional domains are controllable, e.g., inducible.

### Targeting Moieties

The systems may further comprise one or more targeting moieties. The targeting moieties may bind to specific cells or tissues, e.g., by binding to surface receptor proteins. Likewise, the following table provides exemplary targeting moieties that can be used in the practice of the invention an as to each an aspect of the invention provides a system that comprises such a targeting moiety.

TABLE 2
Table 2 - Targeting moieties

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human umbilical |
| aspartic acid-tyrosine- | | vein endothelial cells, tumor |
| cysteine peptide | | angiogenesis |
| (c(RGDyC)-LP) | | |
| ASSHN peptide | | endothelial progenitor cells; anti- |
| | | cancer |
| PR_b peptide | α₅β₁ integrin | cancer cells |
| AG86 peptide | α₆β₄ integrin | cancer cells |
| KCCYSL (P6.1 peptide) | HER-2 receptor | cancer cells |
| affinity peptide LN | Aminopeptidase N | APN-positive tumor |
| (YEVGHRC) | (APN/CD13) | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the systems, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an aspect of the invention wherein the system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao Jet al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference).

Moreover, in view of the teachings herein the skilled artisan can readily select and apply a desired targeting moiety in the practice of the invention as to a lipid entity of the invention. The invention comprehends an embodiment wherein the system comprises a lipid entity having a targeting moiety.

### Nuclear Localization Signals

In some embodiments, the systems and compositions herein further comprises one or more nuclear localization signals (NLSs). The NLS may be capable of driving the accumulation of the components, e.g., Cas and/or transposase(s) to a desired amount in the nucleus of a cell.

In certain embodiments, at least one nuclear localization signal (NLS) is attached to the Cas and/or transposase(s). In some embodiments, one or more C-terminal or N-terminal NLSs are attached (and hence nucleic acid molecule(s) coding for the Cas and/or transposase(s)can include coding for NLS(s) so that the expressed product has the NLS(s) attached or connected). In a preferred embodiment a C-terminal NLS is attached for optimal expression and nuclear targeting in eukaryotic cells, e.g., human cells.

The NLS may be monopartite. In certain cases, the NLS may be bipartite. These types of NLSs can be further classified as either monopartite or bipartite. The two basic amino acid clusters in bipartite NLSs are separated by a short spacer sequence (e.g., about 10 amino acids), while monopartite NLSs are not. In some cases, one or more monopartite NSLs is attached to the Cas and/or transposase(s). In certain cases, one or more bipartite NSLs is attached to the Cas and/or transposase(s). In some cases, one or more monopartite NSLs and one or more bipartite NSLs are attached to the Cas and/or transposase(s).

Non-limiting examples of NLSs include an NLS sequence derived from: the NLS of the SV40 virus large T-antigen, having the amino acid sequence PKKKRKV (SEQ ID NO: 26); the NLS from nucleoplasmin (e.g., the nucleoplasmin bipartite NLS with the sequence KRPAATKKAGQAKKK (SEQ ID NO: 27)); the c-myc NLS having the amino acid sequence PAAKRVKLD (SEQ ID NO: 28) or RQRRNELKRS (SEQ ID NO: 29); the hRNPA1 M9 NLS having the sequence NQSSNFGPMKGGNFGGRSSGPYGGGGQYFAKPRNQGGY (SEQ ID NO: 30); the sequence RMRIZFKNKGKDTAELRRRRVEVSVELRKAKKDEQILKRRNV (SEQ ID NO: 31) of the IBB domain from importin-alpha; the sequences VSRKRPRP (SEQ ID NO: 32) and PPKKARED (SEQ ID NO: 33) of the myoma T protein; the sequence PQPKKKPL (SEQ ID NO: 34) of human p53; the sequence SALIKKKKKMAP (SEQ ID NO: 35) of mouse c-abl IV; the sequences DRLRR (SEQ ID NO: 36) and PKQKKRK (SEQ ID NO: 37) of the influenza virus NS1; the sequence RKLKKKIKKL (SEQ ID NO: 38) of the Hepatitis virus delta antigen; the sequence REKKKFLKRR (SEQ ID NO: 39) of the mouse Mx1 protein; the sequence KRKGDEVDGVDEVAKKKSKK (SEQ ID NO: 40) of the human poly(ADP-ribose) polymerase; and the sequence RKCLQAGMNLEARKTKK (SEQ ID NO: 41) of the steroid hormone receptors (human) glucocorticoid.

In some embodiments, a NLS is a heterologous NLS. For example, the NLS is not naturally present in the molecule (e.g., Cas and/or transposase(s)) it attached to.

In general, strength of nuclear localization activity may derive from the number of NLSs in the nucleic acid-targeting effector protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI).

In some embodiments, a vector described herein (e.g., those comprising polynucleotides encoding Cas and/or transposase(s)) comprise one or more nuclear localization sequences (NLSs), such as about or more than about 1, 2, 3, 4, 5, 6, 7, 8, 9, 10, or more NLSs. More particularly, vector comprises one or more NLSs not naturally present in the Cas and/or transposase(s). Most particularly, the NLS is present in the vector 5′ and/or 3′ of the Cas and/or transposase(s) sequence. In some embodiments, the Cas and/or transposase(s) comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In certain embodiments, other localization tags may be fused to the Cas and/or transposase(s), such as without limitation for localizing to particular sites in a cell, such as organelles, such mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

### Heterologous Components

In some embodiments, the components in the system may be heterologous, i.e., they do not naturally occur together in the same cell or an organism. For example, one or more of: Cas protein(s), transposase(s), other functional domain(s), guide molecules, donor polynucleotides, and target sequences, may be heterologous in view of the other components in the system (i.e., they do not naturally occur together with other components of the systems in the same cell or an organism).

In some examples, the system comprises one or more heterologous guide molecules. The heterologous guide molecules may not naturally occur in the same cell or organism with Cas protein(s), transposase(s) in the system. Such a guide molecule may comprise a heterologous guide sequence, which does not naturally occur in the same molecule with the rest of the guide molecule. In some examples, the guide molecule may not occur in nature, e.g., may be artificially synthesized.

In some examples, the system may comprise one or more heterologous donor polynucleotides. The heterologous donor polynucleotides may not naturally occur in the same cell or organism with a other components in the system. Such a donor polynucleotides may comprise a heterologous insertion sequence, which does not naturally occur in the same molecule with the rest of the guide molecule. In some examples, the heterologous donor polynucleotides may not occur in nature, e.g., may be artificially synthesized.

### Chimeric Systems

In some embodiments, the systems comprise one or more Tn7 transposase is derived from a first species and the one or more Type I-B Cas proteins is derived from a second species different from the first species. In some examples, the systems have two TnsD1s, one from the first species and the other from the second species. In some examples, the systems comprise Cas5, Cas6, Cas7, Cas8, crRNA and TnsD1 from a first species and TnsD1, TnsC, TnsB, TnsA and donor polynucleotide from a second species. In some cases, the systems may further comprise Cas6 and Cas7 of the second species.

In one example, the system comprise Cas5, Cas6, Cas7, Cas8, crRNA and TnsD1 of t24 and TnsD1, TnsC, TnsB, TnsA and donor polynucleotide (and optionally Cas6 and Cas7) of IB29. In one example, the system comprise Cas5, Cas6, Cas7, Cas8, crRNA and TnsD1 of t24 and TnsD1, TnsC, TnsB, TnsA and donor polynucleotide (and optionally Cas6 and Cas7) of IB18. In one example, the system comprise Cas5, Cas6, Cas7, Cas8, crRNA and TnsD1 of IB29 and TnsD1, TnsC, TnsB, TnsA and donor polynucleotide (and optionally Cas6 and Cas7) of t24. In one example, the system comprise Cas5, Cas6, Cas7, Cas8, crRNA and TnsD1 of IB29 and TnsD1, TnsC, TnsB, TnsA and donor polynucleotide (and optionally Cas6 and Cas7) of IB18. In one example, the system comprise Cas5, Cas6, Cas7, Cas8, crRNA and TnsD1 of IB18 and TnsD1, TnsC, TnsB, TnsA and donor polynucleotide (and optionally Cas6 and Cas7) of t24. In one example, the system comprise Cas5, Cas6, Cas7, Cas8, crRNA and TnsD1 of IB18 and TnsD1, TnsC, TnsB, TnsA and donor polynucleotide (and optionally Cas6 and Cas7) of IB29.

### Polynucleotides and Vectors

The systems herein may comprise one or more polynucleotides. The polynucleotide(s) may comprise coding sequences of Cas protein(s), transposase(s), guide molecule(s), donor polynucleotide(s), or any combination thereof. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. It may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Aspects of the disclosure relate to polynucleotide molecules that encode one or more components of the systems as referred to in any embodiment herein. In certain embodiments, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In certain embodiments, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In certain embodiments, the 5′ and/or 3′ end of the isolated polynucleotide sequence may be modified to improve the stability of the sequence of actively avoid degradation. In certain embodiments, the isolated polynucleotide sequence may be comprised in a bacteriophage. In other embodiments, the isolated polynucleotide sequence may be contained in Agrobacterium species. In certain embodiments, the isolated polynucleotide sequence is lyophilized.

### Codon Optimization

Aspects of the disclosure relate to polynucleotide molecules that encode one or more components of the systems as described in any of the embodiments herein, wherein at least one or more regions of the polynucleotide molecule may be codon optimized for expression in a eukaryotic cell. In certain embodiments, the polynucleotide molecules that encode one or more components of the systems as described in any of the embodiments herein are optimized for expression in a mammalian cell or a plant cell.

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, an enzyme coding sequence encoding a Cas protein and/or transposase is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Method of Inserting Polynucleotides

The present disclosure further provides methods of inserting a polynucleotide into a target nucleic acid in a cell, which comprises introducing into a cell: (a) one or more transposases (e.g., CRISPR-associated transposases) or functional fragments thereof, (b) a nucleotide-binding molecule. In some examples, the present disclosure provides methods of inserting a donor polynucleotide into a target polynucleotide in a cell, the method comprises introducing to the cell: one or more CRISPR-associated Tn7 transposases or functional fragments thereof; one or more Type I-B Cas proteins; and a guide molecule capable of complexing with the Type I-B Cas protein and directing binding of the guide-Cas protein complex to a target polynucleotide.

The one or more of components (a), (b) may be expressed from a nucleic acid operably linked to a regulatory sequence that is expressed in the cell. The one or more of components (a), (b) is introduced in a particle. The particle comprises a ribonucleoprotein (RNP). The cell is a prokaryotic cell. The cell is a eukaryotic cell. The cell is a mammalian cell, a cell of a non-human primate, or a human cell. The cell is a plant cell.

In some cases, the method of inserting a donor polynucleotide into a target polynucleotide in a cell, which comprises introducing into the cell: one or more transposases (e.g., CRISPR-associated transposases), a Cas protein; and a guide molecule capable of complexing with the Cas protein and directing sequence specific binding of the guide-Cas protein complex to a target sequence of the target nucleic acid. The one or more CRISPR-associated transposons may comprise one or more transposases and a donor polynucleotide to be inserted.

### Immune Orthogonal Orthologs

In some embodiments, when one or more components of the systems (e.g., transposases, nucleotide-binding molecules) herein need to be expressed or administered in a subject, immunogenicity of the components may be reduced by sequentially expressing or administering immune orthogonal orthologs of the components of the transposon complexes to the subject. As used herein, the term “immune orthogonal orthologs” refer to orthologous proteins that have similar or substantially the same function or activity, but have no or low cross-reactivity with the immune response generated by one another. In some embodiments, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host's immune system (e.g., by activated CTLs). In some examples, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively or additionally, immune overlap among candidates may be assessed by determining B-cell epitopes for the candidate orthologs. In one example, immune orthogonal orthologs may be identified using the method described in Moreno A M et al., BioRxiv, published online Jan. 10, 2018, doi: doi.org/10.1101/245985.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3):273-85, which are incorporated by reference herein in their entireties.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33(11):1162-4.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in WO 2008042156, US 20130185823, and WO2015089419.

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, p7RY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

In some embodiments, the components (or coding sequences thereof) in a composition or system herein may be comprised in a single vector. For example, a single vector may comprise coding sequences for one or more CRISPR-associated Tn7 transposases, one or more Cas proteins, and one more guide molecules. In certain embodiments, the components (or coding sequences thereof) in a composition or system herein may be comprised in separate vectors. In some examples, a first vector may comprise coding sequences for one or more CRISPR-associated Tn7 transposases; a second vector may comprise coding sequences for one or more Cas proteins; a third vector may comprise coding sequences for one or more guide molecules. In some examples, a first vector may comprise coding sequences for one or more CRISPR-associated Tn7 transposases and one or more Cas proteins; a second vector may comprise coding sequences for one or more guide molecules. In some examples, a first vector may comprise coding sequences for one or more CRISPR-associated Tn7 transposases; a second vector may comprise coding sequences for one or more Cas proteins and one or more guide molecules. In some examples, a first vector may comprise coding sequences for one or more CRISPR-associated Tn7 transposases and one or more guide molecules; a second vector may comprise coding sequences for one or more Cas proteins.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown as follows:

TABLE 3

| | AAV- | AAV- | AAV- | AAV- | AAV- | AAV- | AAV- | AAV- |
|---|---|---|---|---|---|---|---|---|
| Cell Line | 1 | 2 | 3 | 4 | 5 | 6 | 8 | 9 |
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HIT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2b (e.g., forming DNA/Ca₂₊ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1. full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Applications in General

The compositions, systems, and methods herein may be used for various applications. In general, one or more target polynucleotides may be modified using the compositions, systems, and methods. In some embodiments, the compositions, systems, and methods may be used to generate engineered cells comprising such modified polynucleotide(s). Tissues, organs, organisms, cell lines, compositions (e.g., pharmaceutical compositions), and devices comprising such engineered cell(s) may be generated as well. The engineered cells, tissues, organs, organisms, cell lines, compositions (e.g., pharmaceutical compositions), and devices may be used as a therapeutic (to treat a disease) and/or a diagnostic, produce a biological products, generate a model organism, etc.

In some embodiments, biological products may be obtained from engineered cells, tissues, organs, organisms, cell lines. The biological products may be chemical compounds, nucleic acids, proteins, lipids, carbohydrates, or any combination thereof. In some example, the biological products may be proteins. In some example, the biological products may be RNA. In some example, the biological products may be DNA. The proteins, RNA, and/or DNA of the biological products may be naturally occurring or non-naturally occurring, e.g., comprising one or more mutations. The product may be a mutated protein or product provided by a template.

In some embodiments, the present disclosure provides cells comprising one or more components of the systems herein. The cells may be engineered cells. The cells may be produced using the systems. The present disclosure includes cell lines comprising the engineered cell and/or progeny thereof. The present disclosure includes a plant or animal model comprising the engineered cell and/or progeny thereof. The present disclosure includes a composition comprising the engineered cell. The compositions may be formulated for us as a therapeutic. The cell or progeny thereof may be isolated (e.g., in isolated forms). The cell may comprise a mutation in a protein expressed from a gene comprising the target sequence. The cell may comprise deletion of a genomic region comprising the target sequence. The cell may comprise integration of an exogenous sequence by homology-directed repair. The cell may comprise decreased transcription of a gene associated with the target sequence. The cell may comprise increased transcription of a gene associated with the target sequence. In some cases, a donor polynucleotide may be inserted into to a target polynucleotide in a cell. The insertion of the donor polynucleotide into the target polynucleotide in the cell results in: a) a cell or population of cells comprising altered expression levels of one or more gene products; and/or b) a cell or population of cells that produces and/or secrete an endogenous or non-endogenous biological product or chemical compound.

The present disclosure includes pharmaceutical compositions for treatment of a disease or disorder, comprising the cell or progeny thereof. The treatment may result in genetic changes in one or more cells. The treatment may result in correction of one or more defective genotypes. The treatment may result in improved phenotype.

### Synthetic Biology Applications

The compositions, systems, and methods herein have a wide utility, including in clinical applications. It is envisaged that the programmable polynucleotide targeting systems can be used fused to split proteins of toxic domains for targeted cell death, for instance using cancer-linked RNA as target transcript. Further, pathways involving protein-protein interaction can be influenced in synthetic biological systems with e.g. fusion complexes with the appropriate effectors such as kinases or other enzymes.

The present disclosure expands the reach of synthetic biology by targeting specific diagnostic and therapeutic applications through improvements in genetic circuitry and higher level genetic circuit delivery enhancements.

Provided herein are methods and compositions having industrial, clinical, and other technological utility. Like all engineering-oriented disciplines, provided herein are modularized, flexible platforms that can be tuned for diverse applications. The highly modular and programmable nature of the compositions, systems, and methods herein can be used as a platform technology in synthetic biology. For example, in alternative embodiments, split enzymes are fused to Cas proteins whose activity is reconstituted upon binding to a target polynucleotide such as complementation of split death-inducing proteins after detection of a cancer-linked RNA or DNA. In alternative embodiments, pathways involving successive protein/protein interactions are re-engineered by using RNA or DNA to scaffold interactions among exemplary Cas fusion proteins as provided herein. In alternative embodiments, scaffold proteins as provided herein can bind kinases and their substrates to strongly influence the output of a signaling pathway, and exemplary Cas polypeptides are used in the scaffolding of protein/protein interactions to control signaling in a gene expression-dependent manner. Another group used tethering of enzymes involved in the production of the drug precursor mevalonate, thereby increasing production of this small molecule (Dueber J E, Wu G C, Malmirchegini G R, Moon T S, et al. 2009. Synthetic protein scaffolds provide modular control over metabolic flux. Nat Biotechnol 27: 753-9). In principle, strong co-binding of exemplary Cas fusion proteins on a target RNA provides a new level of control over successive protein interactions or shuttling of metabolites.

The emerging field of synthetic biology has produced toolbox of genetic regulatory systems that can be applied in basic research and for therapeutic applications. Synthetic biology strategies can create opportunities to change basic research approaches and improve therapeutic treatments for treating injuries and diseases. The complexity of cell signaling networks can be simplified by considering genetic networks composed of subsets of simpler parts, or modules. This simplification is the foundation of synthetic biology, where engineering paradigms are applied in rational and systematic ways to produce predictable and robust systems for understanding or controlling cellular function. This approach entails reprogramming cells to perform in predictable ways. Towards this end, genetic circuits have been built out of DNA and RNA that enable cells to perform Boolean logic functions ranging from memory, and mathematical computations to higher-order cellular functions like cancer cell identification, controlling T cell populations, and reporting on the microenvironment. The engineered gene circuits underlying these functions include genetic switches, oscillators, digital logic gates, and cell counters and have been designed to regulate gene expression in dynamic and predictable ways. While the majority of work in synthetic biology has been in simple organisms such as yeast and bacteria, however, the therapeutic potential of cells carrying engineered genetic circuits has spurred interest in using synthetic biology to remediate or control human disease. This is in part based upon the premise that effective cell therapies require precise temporal and spatial regulation of gene expression, which can be easily controlled by using genetic circuits.

Approaches in synthetic biology as disclosed herein can be used to reprogram cells with genetic circuits to probe the CNS during the onset and progression of neurodegenerative diseases. Such an approach will enable a better understanding of the interactions between neuroinflammation and neurodegeneration that will have a major impact on the broad range of neurological disorders.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fungus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In some embodiments, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi:10.1186/1746-4811-9-39); Brooks, “Efficient gene editing in tomato in the first generation using the CRISPR-Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety. Aspects of utilizing the compositions, systems, and methods may be analogous to the use of the CRISPR-Cas (e.g. CRISPR-Cas9) system in plants, and mention is made of the University of Arizona website “CRISPR-PLANT” (www.genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g. genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and, the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172(1):62-77.

The components (e.g., Cas proteins) in the compositions and systems may further comprise one or more functional domains described herein. In some examples, the functional domains may be an exonuclease. Such exonuclease may increase the efficiency of the Cas proteins' function, e.g., mutagenesis efficiency. An example of the functional domain is Trex2, as described in Weiss T et al., www.biorxiv.org/content/10.1101/2020.04.11.037572v1, doi: https://doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g. those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In some embodiments, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of amphora, anabaena, anikstrodesmis, botryococcus, chaetoceros, chlamydomonas, chlorella, chlorococcum, cyclotella, cylindrotheca, dunaliella, emiliana, euglena, hematococcus, isochrysis, monochrysis, monoraphidium, nannochloris, nannnochloropsis, navicula, nephrochloris, nephroselmis, nitzschia, nodularia, nostoc, oochromonas, oocystis, oscillartoria, pavlova, phaeodactylum, playtmonas, pleurochrysis, porhyra, pseudoanabaena, pyramimonas, stichococcus, synechococcus, synechocystis, tetraselmis, thalassiosira, and trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a Cas protein, a light-responsive cytochrome heterodimer (e.g. from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize 1n2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the Cas gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In some embodiments, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or Cas enzyme in a plant cell; a 5′ untranslated region to enhance expression; an intron element to further enhance expression in certain cells, such as monocot cells; a multiple-cloning site to provide convenient restriction sites for inserting the guide RNA and/or the Cas gene sequences and other desired elements; and a 3′ untranslated region to provide for efficient termination of the expressed transcript.

### Transient Expression in Plants

In some embodiments, the components of the compositions and systems may be transiently expressed in the plant cell. In some examples, the compositions and systems may modify a target nucleic acid only when both the guide RNA and the Cas protein are present in a cell, such that genomic modification can further be controlled. As the expression of the Cas protein is transient, plants regenerated from such plant cells typically contain no foreign DNA. In certain examples, the Cas protein is stably expressed and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7):682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In some embodiments, it is envisaged that the compositions and systems are used to specifically modify chloroplast genes or to ensure expression in the chloroplast. The compositions and systems (e.g., Cas proteins, guide molecules, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61: 157-180, and US 20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, guide molecules targeting one or more locations in a genome may be provided and introduced into plant cells together with the Cas effector protein. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerance and the method is a method for the generation of stress-tolerant crop varieties.

In some embodiments, the compositions, systems, and methods are used to modify endogenous genes or to modify their expression. The expression of the components may induce targeted modification of the genome, either by direct activity of the Cas nuclease and optionally introduction of template DNA, or by modification of genes targeted. The different strategies described herein above allow Cas-mediated targeted genome editing without requiring the introduction of the components into the plant genome.

In some cases, the modification may be performed without the permanent introduction into the genome of the plant of any foreign gene, including those encoding CRISPR components, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In some embodiments, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In some embodiments, genes that confer resistance to pests or diseases may be introduced to plants. In cases there are endogenous genes that confer such resistance in a plants, their expression and function may be enhanced (e.g., by introducing extra copies, modifications that enhance expression and/or activity).

Examples of genes that confer resistance include plant disease resistance genes (e.g., Cf-9, Pto, RSP2, S1DMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In some embodiments, compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In some embodiments, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In some embodiments, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In some embodiments, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In some embodiments, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g. by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., w-3 fatty acids, Conjugated linoleic acid, GLA), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g. as many as six, such as in wheat). In some cases, the compositions, systems, and methods may be can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-B1 and TaMLO-D1 nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In some embodiments, the compositions, systems, and methods are used to reduce ethylene production. In some examples, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression.

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In some embodiments, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In particular embodiments, the methods provided herein are used to reduce or inhibit expression of the vacuolar invertase gene (VInv), which encodes a protein that breaks down sucrose to glucose and fructose.

### Reducing Allergens in Plants

In some embodiments, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11(3):222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12(3):321-342; and Kim Y J, et al., Trends Plant Sci. 2018 January; 23(1):53-65.

### Increasing the Fertility Stage in Plants

In some embodiments, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In some embodiments, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January; 49(1):162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl acyl-carrier protein synthase III, glycerol-3-phosphate dehydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyl protein thioesterase, or malic enzyme activities.

In further embodiments it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and WO 2015086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ‘tesA, tesB, fatB, fatB2, fatB3, fatA1, or fatA), acyl-CoA synthases (e.g., fadD, JadK, BH3103, pfl-4354, EAV15023, fadD1, fadD2, RPC_4074, fadDD35, fadDD22, faa39), ester synthases (e.g., synthase/acyl-CoA:diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In some embodiments, plants may be modified to produce organic acids such as lactic acid. The plants may produce organic acids using sugars, pentose or hexose sugars. To this end, one or more genes may be introduced (e.g., and overexpressed) in the plants. An example of such genes include LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (l-ldh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In some embodiments, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In particular embodiments, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In some embodiments, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome—Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, 51 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In some embodiments, the fungal cell is a yeast cell. A yeast cell refers to any fungal cell within the phyla Ascomycota and Basidiomycota. Examples of yeasts include budding yeast, fission yeast, and mold, S. cerevisiae, Kluyveromyces marxianus, Issatchenkia orientalis, Candida spp. (e.g., Candida albicans), Yarrowia spp. (e.g., Yarrowia hpolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In some embodiments, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In some embodiments, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the CRISPR system described herein may take advantage of using certain fungal cell types.

In some embodiments, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In some embodiments, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof, may be introduced to fungi cells using the delivery systems and methods herein. Examples of delivery systems include lithium acetate treatment, bombardment, electroporation, and those described in Kawai et al., 2010, Bioeng Bugs. 2010 November-December; 1(6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

### Biofuel and Materials Production by Fungi

In some embodiments, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced in to fungi In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S. J., et al. (2011) Proc. Natl. Acad. Sci. USA 108(2):504-9 and Galazka, J. M., et al. (2010) Science 330(6000):84-6; Jakoĉiũnas T et al., Metab Eng. 2015 March; 28:213-222; Stovicek V, et al., FEMS Yeast Res. 2017 Aug. 1; 17(5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications of the CRISPR-Cas System in Plants

Further applications of the compositions, systems, and methods on plants and fungi include visualization of genetic element dynamics (e.g., as described in Chen B, et al., Cell. 2013 Dec. 19; 155(7):1479-91), targeted gene disruption positive-selection in vitro and in vivo (as described in Malina A et al., Genes Dev. 2013 Dec. 1; 27(23):2602-14), epigenetic modification such as using fusion of Cas and histone-modifying enzymes (e.g., as described in Rusk N, Nat Methods. 2014 January; 11(1):28), identifying transcription regulators (e.g., as described in Waldrip Z J, Epigenetics. 2014 September; 9(9):1207-11), anti-virus treatment for both RNA and DNA viruses (e.g., as described in Price A A, et al., Proc Natl Acad Sci USA. 2015 May 12; 112(19):6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112(36):11211-6; Anton T, et al., Nucleus. 2014 March-April; 5(2):163-72), self-cleavage of the CRISPR system for controlled inactivation/activation (e.g., as described Sugano S S et al., Plant Cell Physiol. 2014 March; 55(3):475-81), multiplexed gene editing (as described in Kabadi A M et al., Nucleic Acids Res. 2014 Oct. 29; 42(19):e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August; 8(8):1274-84), regulation of non-coding genes and sequences (e.g., as described in Lowder L G, et al., Plant Physiol. 2015 October; 169(2):971-85), editing genes in trees (e.g., as described in Belhaj K et al., Plant Methods. 2013 Oct. 11; 9(1):39; Harrison MINI, et al., Genes Dev. 2014 Sep. 1; 28(17):1859-72; Zhou X et al., New Phytol. 2015 October; 208(2):298-301), introduction of mutations for resistance to host-specific pathogens and pests.

Additional examples of modifications of plants and fungi that may be performed using the compositions, systems, and methods include those described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In some embodiments, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0—genome editing for fitter, healthier, and more productive farmed animals. Genome Biol. 2018 Nov. 26; 19(1):204; Lillico S, Agricultural applications of genome editing in farmed animals. Transgenic Res. 2019 August; 28(Suppl 2):57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats horses, wolfs, rabbits, ferrets, gerbils, hamsters, chinchillas, fancy rats, guinea pigs, canaries, parakeets, and parrots.

In some embodiments, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g. in pigs (such as described in Li M, et al., Genetics. 2018; 8:1747-54). Phytase (e.g., from E coli) xylanase (e.g., from Aspergillus niger), beta-glucanase (e.g., from Bacillus licheniformis) may be introduced to reduce the environmental impact through phosphorous and nitrogen release reduction, e.g. in pigs (such as described in Golovan S P, et al., Nat Biotechnol. 2001; 19:741-5; Zhang X et al., elife. 2018). shRNA decoy may be introduced to induce avian influenza resilience e.g. in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLoS One. 2017; 12:e0169317). CD163 may be modified (e.g., inactivated or removed) to introduce PRRSV resilience in pigs (such as described in Prather R S et al., Sci Rep. 2017 Oct. 17; 7(1):13371). Similar approaches may be used to inhibit or remove viruses and bacteria (e.g., Swine Influenza Virus (SIV) strains which include influenza C and the subtypes of influenza A known as H1N1, H1N2, H2N1, H3N1, H3N2, and H2N3, as well as pneumonia, meningitis and oedema) that may be transmitted from animals to humans.

In some embodiments, one or more genes may be modified or edited for disease resistance and production traits. Myostatin (e.g., GDF8) may be modified to increase muscle growth, e.g., in cow, sheep, goat, catfish, and pig (such as described in Crispo M et al., PLoS One. 2015; 10:e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISS1R may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2) RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMP1 may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December; 7(6):580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111(20):7260-5; and Schomberg et al. FASEB Journal, April 2016; 30(1):Suppl 571.1.

SNPs in the animals may be modified. Examples of methods and approaches include those described Tan W. et al., Proc Natl Acad Sci USA. 2013 Oct. 8; 110(41):16526-31; Mali P, et al., Science. 2013 Feb. 15; 339(6121):823-6.

Stem cells (e.g., induced pluripotent stem cells) may be modified and differentiated into desired progeny cells, e.g., as described in Heo Y T et al., Stem Cells Dev. 2015 Feb. 1; 24(3):393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

### Models of Genetic and Epigenetic Conditions

A method of the invention may be used to create a plant, an animal or cell that may be used to model and/or study genetic or epigenetic conditions of interest, such as a through a model of mutations of interest or a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of components of the system; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the systems and methods herein on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

Several disease models have been specifically investigated. These include de novo autism risk genes CHD8, KATNAL2, and SCN2A; and the syndromic autism (Angelman Syndrome) gene UBE3A. These genes and resulting autism models are of course preferred, but serve to show the broad applicability of the invention across genes and corresponding models. An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, ß-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphorimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent:polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2a). Alternatively, these antibodies can be generated using conventional polyclonal or monoclonal antibody technologies by immunizing a host animal or an antibody-producing cell with a target protein that exhibits the desired post-translational modification.

In practicing the subject method, it may be desirable to discern the expression pattern of an protein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a millisecond.

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The target polynucleotide of the system herein can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence) Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523 (7561): 481-5. doi: 10.1038/nature14592.

The target polynucleotide of the system may include a number of disease-associated genes and polynucleotides as well as signaling biochemical pathway-associated genes and polynucleotides as listed in U.S. provisional patent applications 61/736,527 and 61/748,427 having Broad reference BI-2011/008/WSGR Docket No. 44063-701.101 and BI-2011/008/WSGR Docket No. 44063-701.102 respectively, both entitled SYSTEMS METHODS AND COMPOSITIONS FOR SEQUENCE MANIPULATION filed on Dec. 12, 2012 and Jan. 2, 2013, respectively, and PCT Application PCT/US2013/074667, entitled DELIVERY, ENGINEERING AND OPTIMIZATION OF SYSTEMS, METHODS AND COMPOSITIONS FOR SEQUENCE MANIPULATION AND THERAPEUTIC APPLICATIONS, filed Dec. 12, 2013, the contents of all of which are herein incorporated by reference in their entirety.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

### Therapeutic Applications

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In some embodiments, the method of treatment or prevention can include using a composition, system, or component thereof to modify a polynucleotide of an infectious organism (e.g. bacterial or virus) within a subject or cell thereof. In some embodiments, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple Cas effectors. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the Cas effector(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the Cas effector(s) advantageously encoding and expressing in vivo the remaining portions of the composition, system, (e.g., RNA, guides); advantageously in some embodiments the CRISPR enzyme is a catalytically inactive Cas effector and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

Thus, also described herein are methods of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiment or the cell of any one of the above embodiment in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g. guide(s) RNA(s) or sgRNA(s)), such as those mediated by a composition, system, or a component thereof described elsewhere herein. In some embodiments, the modifications can include the introduction, deletion, or substitution of nucleotides at a target or random sequence of said cell(s) via a composition, system, or technique.

The target sequences of polynucleotides to be modified to treat or prevent disease are described in greater detail below.

As is also discussed elsewhere herein, the composition, system, can include a template polynucleotide (also referred to herein as template nucleic acids or template sequence). In an embodiment, the template nucleic acid alters the structure of the target position by participating in homologous recombination. In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid can include sequence that corresponds to a site on the target sequence that is cleaved, nicked, or otherwise modified by one or more Cas effector mediated cleavage event(s). In an embodiment, the template nucleic acid can include sequence that corresponds to both, a first site on the target sequence that is cleaved, nicked, or otherwise modified in a first Cas effector mediated event, and a second site on the target sequence that is cleaved in a second Cas effector mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence. In an embodiment, the template nucleic acid may be 20+/−10, 30+/−10, 40+/−10, 50+/−10, 60+/−10, 70+/−10, 80+/−10, 9 0+/−10, 100+/−10, 110+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In certain embodiments, a template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In some embodiments, the composition, system, or component thereof can promote Non-Homologous End-Joining (NHEJ). In some embodiments, modification of a polynucleotide by a composition, system, or a component thereof, such as a diseased polynucleotide, can include NHEJ. In some embodiments, promotion of this repair pathway by the composition, system, or a component thereof can be used to target gene or polynucleotide specific knock-outs and/or knock-ins. In some embodiments, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In some embodiments thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, composition, system, mediated NHEJ can be used in the method to delete small sequence motifs. In some embodiments, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and Cas effector generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more Cas nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D1 OA mutation) can be delivered with a pair of guide RNAs targeting a site of interest. Guide sequences and strategies to minimize toxicity and off-target effects can be as in WO 2014/093622 (PCT/US2013/074667); or, via mutation. Others are as described elsewhere herein.

Typically, in the context of an endogenous CRISPR or CAST system, formation of a CRISPR or CAST complex (comprising a guide sequence hybridized to a target sequence and complexed with one or more Cas proteins) results in cleavage, nicking, and/or another modification of one or both strands in or near (e.g. within 1, 2, 3, 4, 5, 6, 7, 8, 9, 10, 20, 50, or more base pairs from) the target sequence. In some embodiments, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), can also form part of a CRISPR complex, such as by hybridization along at least a portion of the tracr sequence to all or a portion of a tracr mate sequence that is operably linked to the guide sequence.

In some embodiments, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some of these embodiments, the composition, system, or component thereof can be or include a CRISPR-Cas effector complexed with a guide sequence. In some embodiments, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In some embodiments, modification of transcription can include decreasing transcription of a target polynucleotide. In some embodiments, modification can include increasing transcription of a target polynucleotide. In some embodiments, the method includes repairing said cleaved target polynucleotide by homologous recombination with an exogenous template polynucleotide, wherein said repair results in a modification such as, but not limited to, an insertion, deletion, or substitution of one or more nucleotides of said target polynucleotide. In some embodiments, said modification results in one or more amino acid changes in a protein expressed from a gene comprising the target sequence. In some embodiments, the modification imparted by the composition, system, or component thereof provides a transcript and/or protein that can correct a disease or a symptom thereof, including but not limited to, any of those described in greater detail elsewhere herein.

In some embodiments, the method of treating or preventing a disease can include delivering one or more vectors or vector systems to a cell, such as a eukaryotic or prokaryotic cell, wherein one or more vectors or vector systems include the composition, system, or component thereof. In some embodiments, the vector(s) or vector system(s) can be a viral vector or vector system, such as an AAV or lentiviral vector system, which are described in greater detail elsewhere herein. In some embodiments, the method of treating or preventing a disease can include delivering one or more viral particles, such as an AAV or lentiviral particle, containing the composition, system, or component thereof. In some embodiments, the viral particle has a tissue specific tropism. In some embodiments, the viral particle has a liver, muscle, eye, heart, pancreas, kidney, neuron, epithelial cell, endothelial cell, astrocyte, glial cell, immune cell, or red blood cell specific tropism.

It will be understood that the composition, system, according to the invention as described herein, such as the composition, system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In some embodiments, the composition, system, and/or component thereof described herein can be used to treat and/or prevent a circulatory system disease. Exemplary disease is provided, for example, in Tables 3 and 4. In some embodiments the plasma exosomes of Wahlgren et al. (Nucleic Acids Research, 2012, Vol. 40, No. 17 e130) can be used to deliver the composition, system, and/or component thereof described herein to the blood. In some embodiments, the circulatory system disease can be treated by using a lentivirus to deliver the composition, system, described herein to modify hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi:10.4061/2011/987980, which can be adapted for use with the composition, system, herein in view of the description herein). In some embodiments, the circulatory system disorder can be treated by correcting HSCs as to the disease using a composition, system, herein or a component thereof, wherein the composition, system, optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia”, Nature 467, 318-322 (16 Sep. 2010) doi:10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene ((3A-T87Q); and Xie et al., “Seamless gene correction of β-thalassaemia mutations in patient-specific iPSCs using CRISPR/Cas9 and piggyback” Genome Research gr.173427.114 (2014) http://www.genome.org/cgi/doi/10.1101/gr.173427.114 (Cold Spring Harbor Laboratory Press; [1599] Watts, “Hematopoietic Stem Cell Expansion and Gene Therapy” Cytotherapy 13(10):1164-1171. doi:10.3109/14653249.2011.620748 (2011), which can be adapted for use with the composition, system, herein in view of the description herein). In some embodiments, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9):1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,—the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, Il7Ra, CD3, CD4, CD5, CD8 for T cells, etc. Mouse HSC markers: CD34lo/−, SCA-1+, Thy1.1+/lo, CD38+, C-kit+, lin−, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

The CRISPR-Cas (system may be engineered to target genetic locus or loci in HSCs. In some embodiments, the Cas effector(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the Cas effector (e.g., Cas9) protein and the gRNA being admixed. The gRNA and Cas effector (e.g., Cas9) protein mixture can be, for example, admixed with a mixture comprising or consisting essentially of or consisting of surfactant, phospholipid, biodegradable polymer, lipoprotein and alcohol, whereby particles containing the gRNA and Cas effector (e.g. Cas9) protein may be formed. The invention comprehends so making particles and particles from such a method as well as uses thereof. Particles suitable delivery of the CRISRP-Cas systems in the context of blood or circulatory system or HSC delivery to the blood or circulatory system are described in greater detail elsewhere herein.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified can be autologous. In some embodiments, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Diseases of the Brain

In some embodiments, the compositions, systems, described herein can be used to treat diseases of the brain and CNS. Delivery options for the brain include encapsulation of CRISPR enzyme and guide RNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing CRISPR enzyme and guide RNA. For instance, Xia C F and Boado R J, Pardridge W M (“Antibody-mediated targeting of siRNA via the human insulin receptor using avidin-biotin technology.” Mol Pharm. 2009 May-June; 6(3):747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g. Zhang et al. (Mol Ther. 2003 January; 7(1):11-8.)), the teachings of which can be adapted for use with the compositions, systems, herein.

### Treating Hearing Diseases

In some embodiments the composition, system, described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In some embodiments, the composition, system, or modified cells can be delivered to one or both ears for treating or preventing hearing disease or loss by any suitable method or technique. Suitable methods and techniques include, but are not limited to those set forth in US patent application 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In some embodiments, a catheter or pump can be positioned, e.g., in the ear (e.g., the outer, middle, and/or inner ear) of a patient during a surgical procedure. In some embodiments, a catheter or pump can be positioned, e.g., in the ear (e.g., the outer, middle, and/or inner ear) of a patient without the need for a surgical procedure.

In general, the cell therapy methods described in US patent application 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Publication No. 2005/0287127) and Li et al., (U.S. patent Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318(5858):1917-1920 (2007); Nakagawa et al., Nat. Biotechnol. 26:101-106 (2008); and Zaehres and Scholer, Cell 131(5):834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Published application, 20110142917. In some embodiments the pharmaceutical composition is applied to the ear canal. Delivery to the ear may also be referred to as aural or otic delivery.

In some embodiments, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2):180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In some embodiments, the system set forth in Mukherjea et al. (Antioxidants & Redox Signaling, Volume 13, Number 5, 2010) can be adapted for transtympanic administration of the composition, system, or component thereof to the ear. In some embodiments, a dosage of about 2 mg to about 4 mg of CRISPR Cas for administration to a human.

In some embodiments, the system set forth in [Jung et al. (Molecular Therapy, vol. 21 no. 4, 834-841 April 2013) can be adapted for vestibular epithelial delivery of the composition, system, or component thereof to the ear. In some embodiments, a dosage of about 1 to about 30 mg of CRISPR Cas for administration to a human.

### Treating Diseases in Non-Dividing Cells

In some embodiments, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U20S) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas9-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the Cas compositions, systems, described herein.

Thus, reactivation of HR in cells, especially non-dividing, fully differentiated cell types is preferred, in some embodiments. In some embodiments, promotion of the BRCA1-PALB2 interaction is preferred in some embodiments. In some embodiments, the target ell is a non-dividing cell. In some embodiments, the target cell is a neuron or muscle cell. In some embodiments, the target cell is targeted in vivo. In some embodiments, the cell is in G1 and HR is suppressed. In some embodiments, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in some embodiments, especially where the target cells are non-dividing, or where removal and return (ex vivo gene targeting) is problematic, for example neuron or muscle cells. KEAP1 siRNA is available from ThermoFischer. In some embodiments, a BRCA1-PALB2 complex may be delivered to the G1 cell. In some embodiments, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In some embodiments, the disease to be treated is a disease that affects the eyes. Thus, in some embodiments, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In some embodiments, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In some embodiments, for administration to the eye, lentiviral vectors. In some embodiments, the lentiviral vector is an equine infectious anemia virus (EIAV) vector. Exemplary EIAV vectors for eye delivery are described in Balagaan, J Gene Med 2006; 8: 275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In some embodiments, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In some embodiments, the dose can range from about 10⁶ to 10^{9.5} particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In some embodiments, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of CRISPR administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye. In particular, desirable targets are zgc:193933, prdm1a, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In some embodiments, the composition, system, can be used to treat and/or prevent a muscle disease and associated circulatory or cardiovascular disease or disorder. The present invention also contemplates delivering the composition, system, described herein, e.g. Cas effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes are contemplated for systemic administration. See also, e.g., Eulalio et al. (2012) Nature 492: 376 and Somasuntharam et al. (2013) Biomaterials 34: 7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR). Exemplary chromosomal sequences can be found in Table 3.

The compositions, systems, herein can be used for treating diseases of the muscular system. The present invention also contemplates delivering the composition, system, described herein, e.g. Cas (e.g. Cas9 and/or Cas12) effector protein systems, to muscle(s).

In some embodiments, the muscle disease to be treated is a muscle dystrophy such as DMD. In some embodiments, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In some embodiments, exon skipping can be achieved in dystrophin mRNA. In some embodiments, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In some embodiments, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In some embodiments, for treatment of a muscle disease, the method of Bortolanza et al. Molecular Therapy vol. 19 no. 11, 2055-264 November 2011) may be applied to an AAV expressing CRISPR Cas and injected into humans at a dosage of about 2×10¹⁵ or 2×10¹⁶ vg of vector. The teachings of Bortolanza et al., can be adapted for and/or applied to the compositions, systems, described herein.

In some embodiments, the method of Dumonceaux et al. (Molecular Therapy vol. 18 no. 5, 881-887 May 2010) may be applied to an AAV expressing CRISPR Cas and injected into humans, for example, at a dosage of about 10¹⁴ to about 10¹⁵ vg of vector. The teachings of Dumonceaux described herein can be adapted for and/or applied to the compositions, systems, described herein.

In some embodiments, the method of Kinouchi et al. (Gene Therapy (2008) 15, 1126-1130) may be applied to CRISPR Cas systems described herein and injected into a human, for example, at a dosage of about 500 to 1000 ml of a 40 μM solution into the muscle.

In some embodiments, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

### Treating Diseases of the Liver and Kidney

In some embodiments, the composition, system, or component thereof described herein can be used to treat a disease of the kidney or liver. Thus, in some embodiments, delivery of the CRISRP-Cas system or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Peter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the CRISPR Cas system of the present invention contemplating a 1-2 g subcutaneous injection of CRISPR Cas conjugated with cholesterol to a human for delivery to the kidneys. In some embodiments, the method of Molitoris et al. (J Am Soc Nephrol 20: 1754-1764, 2009) can be adapted to the CRISRP-Cas system of the present invention and a cumulative dose of 12-20 mg/kg to a human can be used for delivery to the proximal tubule cells of the kidneys. In some embodiments, the methods of Thompson et al. (Nucleic Acid Therapeutics, Volume 22, Number 4, 2012) can be adapted to the CRISRP-Cas system of the present invention and a dose of up to 25 mg/kg can be delivered via i.v. administration. In some embodiments, the method of Shimizu et al. (J Am Soc Nephrol 21: 622-633, 2010) can be adapted to the CRISRP-Cas system of the present invention and a dose of about of 10-20 μmol CRISPR Cas complexed with nanocarriers in about 1-2 liters of a physiologic fluid for i.p. administration can be used.

Other various delivery vehicles can be used to deliver the composition, system, to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In some embodiments, delivery is to liver cells. In some embodiments, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called “safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted donor template) to be achieved even if only a small fraction of hepatocytes are edited. See sites identified by Wechsler et al. (reported at the 57th Annual Meeting and Exposition of the American Society of Hematology—abstract available online at https://ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In some embodiments, the disease treated or prevented by the composition, system, described herein can be a lung or epithelial disease. The compositions, systems, described herein can be used for treating epithelial and/or lung diseases. The present invention also contemplates delivering the composition, system, described herein, to one or both lungs.

In some embodiments, as viral vector can be used to deliver the composition, system, or component thereof to the lungs. In some embodiments, the AAV is an AAV-1, AAV-2, AAV-5, AAV-6, and/or AAV-9 for delivery to the lungs. (see, e.g., Li et al., Molecular Therapy, vol. 17 no. 12, 2067-277 December 2009). In some embodiments, the MOI can vary from 1×10³ to 4×10⁵ vector genomes/cell. In some embodiments, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized CRISPR Cas, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for Cas (Cas (e.g. Cas9 and/or Cas12)), U6 or H1 promoter for guide RNA): A preferred arrangement is to use a CFTRdelta508 targeting guide, a repair template for deltaF508 mutation and a codon optimized Cas (e.g. Cas9 and/or Cas12) enzyme, with optionally one or more nuclear localization signal or sequence(s) (NLS(s)), e.g., two (2) NLSs.

### Treating Diseases of the Skin

The compositions, systems, described herein can be used for the treatment of skin diseases. The present invention also contemplates delivering the composition, system, described herein, to the skin.

In some embodiments, delivery to the skin (intradermal delivery) of the composition, system, or component thereof can be via one or more microneedles or microneedle containing device. For example, in some embodiments the device and methods of Hickerson et al. (Molecular Therapy—Nucleic Acids (2013) 2, e129) can be used and/or adapted to deliver the composition, system, described herein, for example, at a dosage of up to 300 μl of 0.1 mg/ml CRISPR-Cas (e.g. Cas9 and/or Cas12) system to the skin.

In some embodiments, the methods and techniques of Leachman et al. (Molecular Therapy, vol. 18 no. 2, 442-446 February 2010) can be used and/or adapted for delivery of a CIRPSR-Cas system described herein to the skin.

In some embodiments, the methods and techniques of [1785] Zheng et al. (PNAS, Jul. 24, 2012, vol. 109, no. 30, 11975-11980) can be used and/or adapted for nanoparticle delivery of a CIRPSR-Cas system described herein to the skin. In some embodiments, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in WO2015161276, the disclosure of which is hereby incorporated by reference and described herein below.

Target genes suitable for the treatment or prophylaxis of cancer can include those set forth in Tables 4 and 5. In some embodiments, target genes for cancer treatment and prevention can also include those described in WO2015048577 the disclosure of which is hereby incorporated by reference and can be adapted for and/or applied to the composition, system, described herein.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In some embodiments, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In some embodiments, modifying one or more copies of one or more genes associated with a genetic disease or a disease with a genetic and/or epigenetic aspect in the subject can eliminate a genetic disease or a symptom thereof in the subject. In some embodiments, modifying one or more copies of one or more genes associated with a genetic disease or a disease with a genetic and/or epigenetic aspect in the subject can decrease the severity of a genetic disease or a symptom thereof in the subject. In some embodiments, the compositions, systems, or components thereof can modify one or more genes or polynucleotides associated with one or more diseases, including genetic diseases and/or those having a genetic aspect and/or epigenetic aspect, including but not limited to, any one or more set forth in Table 3. It will be appreciated that those diseases and associated genes listed herein are non-exhaustive and non-limiting. Further some genes play roles in the development of multiple diseases.

TABLE 4
Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, :GA:S1, |
| | | | CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMRI, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tubreous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, ILIA, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1,HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MATIA, OPRL1, IMPA1, CLCN2, |
| | | | DLD, PSMA6, PSMB8, CHI3L1, |
| | | | ALDH1B1, PARP2,STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2,TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT20), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2;Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias (e.g. |
| | | | Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HIRAI, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-XI, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between -92 and - |
| | | | 130 of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | ½ promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In some embodiments, the compositions, systems, or components thereof can be used treat or prevent a disease in a subject by modifying one or more genes associated with one or more cellular functions, such as any one or more of those in Table 5. In some embodiments, the disease is a genetic disease or disorder. In some of embodiments, the composition, system, or component thereof can modify one or more genes or polynucleotides associated with one or more genetic diseases such as any set forth in Table 5.

TABLE 5
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARMI; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLDI; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | EROIL; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; crn2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 orBm3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In some embodiments, the composition, system,(s) or component(s) thereof can be used to diagnose, prognose, treat, and/or prevent an infectious disease caused by a microorganism, such as bacteria, virus, fungi, parasites, or combinations thereof.

In some embodiments, the system(s) or component(s) thereof can be capable of targeting specific microorganism within a mixed population. Exemplary methods of such techniques are described in e.g. Gomaa A A, Klumpe H E, Luo M L, Selle K, Barrangou R, Beisel C L. 2014. Programmable removal of bacterial strains by use of genome-targeting composition, systems. mBio 5:e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In some embodiments, the composition, system,(s) and/or components thereof can be capable of targeting pathogenic and/or drug-resistant microorganisms, such as bacteria, virus, parasites, and fungi. In some embodiments, the composition, system,(s) and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In some embodiments, the pathogenic bacteria that can be targeted and/or modified by the composition, system,(s) and/or component(s) thereof described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. Faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streptococcus (S. agalactiaee, S. pneumoniae, S. pyogenes), Treponema (T. pallidum), Ureeaplasma (e.g. U. urealyticum), Vibrio (e.g. V. cholerae), Yersinia (e.g. Y pestis, Y, enteerocolitica, and Y, pseudotuberculosis).

In some embodiments, the pathogenic virus that can be targeted and/or modified by the composition, system,(s) and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g. Adenovirus), Herpesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatitis D virus, Reoviridae (e.g. Rotavirus, Orbivirus, Coltivirus, Banna virus).

In some embodiments, the pathogenic fungi that can be targeted and/or modified by the composition, system,(s) and/or component(s) thereof described herein include, but are not limited to, those of the genus Candida (e.g. C. albicans), Aspergillus (e.g. A. fumigatus, A. flavus, A. clavatus), Cryptococcus (e.g. C. neoformans, C. gattii), Histoplasma (H. capsulatum), Pneumocystis (e.g. P. jiroveecii), Stachybotrys (e.g. S. chartarum).

In some embodiments, the pathogenic parasites that can be targeted and/or modified by the composition, system,(s) and/or component(s) thereof described herein include, but are not limited to, protozoa, helminths, and ectoparasites. In some embodiments, the pathogenic protozoa that can be targeted and/or modified by the composition, system,(s) and/or component(s) thereof described herein include, but are not limited to, those from the groups Sarcodina (e.g. ameba such as Entamoeba), Mastigophora (e.g. flagellates such as Giardia and Leishmania), Cilophora (e.g. ciliates such as Balantidum), and sporozoa (e.g. plasmodium and Cryptosporidium). In some embodiments, the pathogenic helminths that can be targeted and/or modified by the composition, system,(s) and/or component(s) thereof described herein include, but are not limited to, flatworms (platyhelminths), thorny-headed worms (acanthoceephalins), and roundworms (nematodes). In some embodiments, the pathogenic ectoparasites that can be targeted and/or modified by the composition, system,(s) and/or component(s) thereof described herein include, but are not limited to, ticks, fleas, lice, and mites.

In some embodiments, the pathogenic parasite that can be targeted and/or modified by the composition, system,(s) and/or component(s) thereof described herein include, but are not limited to, Acanthamoeba spp., Balamuthia mandrillaris, Babesiosis spp. (e.g. Babesia B. divergens, B. bigemina, B. equi, B. microfti, B. duncani), Balantidiasis spp. (e.g. Balantidium coli), Blastocystis spp., Cryptosporidium spp., Cyclosporiasis spp. (e.g. Cyclospora cayetanensis), Dientamoebiasis spp. (e.g. Dientamoeba fragilis), Amoebiasis spp. (e.g. Entamoeba histolytica), Giardiasis spp. (e.g. Giardia lamblia), Isosporiasis spp. (e.g. Isospora belli), Leishmania spp., Naegleria spp. (e.g. Naegleria fowleri), Plasmodium spp. (e.g. Plasmodium falciparum, Plasmodium vivax, Plasmodium ovale curtisi, Plasmodium ovale wallikeri, Plasmodium malariae, Plasmodium knowlesi), Rhinosporidiosis spp. (e.g. Rhinosporidium seeberi), Sarcocystosis spp. (e.g. Sarcocystis bovihominis, Sarcocystis suihominis), Toxoplasma spp. (e.g. Toxoplasma gondii), Trichomonas spp. (e.g. Trichomonas vaginalis), Trypanosoma spp. (e.g. Trypanosoma brucei), Trypanosoma spp. (e.g. Trypanosoma cruzi), Tapeworm (e.g. Cestoda, Taenia multiceps, Taenia saginata, Taenia solium), Diphyllobothrium latum spp., Echinococcus spp. (e.g. Echinococcus granulosus, Echinococcus multilocularis, E. vogeli, E. oligarthrus), Hymenolepis spp. (e.g. Hymenolepis nana, Hymenolepis diminuta), Bertiella spp. (e.g. Bertiella mucronata, Bertiella studeri), Spirometra (e.g. Spirometra erinaceieuropaei), Clonorchis spp. (e.g. Clonorchis sinensis; Clonorchis viverrini), Dicrocoelium spp. (e.g. Dicrocoelium dendriticum), Fasciola spp. (e.g. Fasciola hepatica, Fasciola gigantica), Fasciolopsis spp. (e.g. Fasciolopsis buski), Metagonimus spp. (e.g. Metagonimus yokogawai), Metorchis spp. (e.g. Metorchis conjunctus), Opisthorchis spp. (e.g. Opisthorchis viverrini, Opisthorchis felineus), Clonorchis spp. (e.g. Clonorchis sinensis), Paragonimus spp. (e.g. Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g. Schistosoma mansoni, Schistosoma haematobium, Schistosoma japonicum, Schistosoma mekongi, and Schistosoma intercalatum), Echinostoma spp. (e.g. E. echinatum), Trichobilharzia spp. (e.g. Trichobilharzia regent), Ancylostoma spp. (e.g. Ancylostoma duodenale), Necator spp. (e.g. Necator americanus), Angiostrongylus spp., Anisakis spp., Ascaris spp. (e.g. Ascaris lumbricoides), Baylisascaris spp. (e.g. Baylisascaris procyonis), Brugia spp. (e.g. Brugia malayi, Brugia timori), Dioctophyme spp. (e.g. Dioctophyme renale), Dracunculus spp. (e.g. Dracunculus medinensis), Enterobius spp. (e.g. Enterobius vermicularis, Enterobius gregorii), Gnathostoma spp. (e.g. Gnathostoma spinigerum, Gnathostoma hispidum), Halicephalobus spp. (e.g. Halicephalobus gingivalis), Loa loa spp. (e.g. Loa loa filaria), Mansonella spp. (e.g. Mansonella streptocerca), Onchocerca spp. (e.g. Onchocerca volvulus), Strongyloides spp. (e.g. Strongyloides stercoralis), Thelazia spp. (e.g. Thelazia californiensis, Thelazia callipaeda), Toxocara spp. (e.g. Toxocara canis, Toxocara cati, Toxascaris leonine), Trichinella spp. (e.g. Trichinella spiralis, Trichinella britovi, Trichinella nelsoni, Trichinella nativa), Trichuris spp. (e.g. Trichuris trichiura, Trichuris vulpis), Wuchereria spp. (e.g. Wuchereria bancrofti), Dermatobia spp. (e.g. Dermatobia hominis), Tunga spp. (e.g. Tunga penetrans), Cochliomyia spp. (e.g. Cochliomyia hominivorax), Linguatula spp. (e.g. Linguatula serrata), Archiacanthocephala sp., Moniliformis sp. (e.g. Moniliformis moniliformis), Pediculus spp. (e.g. Pediculus humanus capitis, Pediculus humanus humanus), Pthirus spp. (e.g. Pthirus pubis), Arachnida spp. (e.g. Trombiculidae, Ixodidae, Argaside), Siphonaptera spp (e.g. Siphonaptera: Pulicinae), Cimicidae spp. (e.g. Cimex lectularius and Cimex hemipterus), Diptera spp., Demodex spp. (e.g. Demodex folliculorum/brevis/canis), Sarcoptes spp. (e.g. Sarcoptes scabiei), Dermanyssus spp. (e.g. Dermanyssus gallinae), Ornithonyssus spp. (e.g. Ornithonyssus sylviarum, Ornithonyssus bursa, Ornithonyssus bacoti), Laelaps spp. (e.g. Laelaps echidnina), Liponyssoides spp. (e.g. Liponyssoides sanguineus).

In some embodiments, the gene targets can be any of those as set forth in Table 1 of Strich and Chertow. 2019. J. Clin. Microbio. 57:4 e01307-18, which is incorporated herein as if expressed in its entirety herein.

In some embodiments, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In some embodiments, delivery of the composition, system, occurs in vivo (i.e. in the subject being treated). In some embodiments occurs by an intermediary, such as microorganism or phage that is non-pathogenic to the subject but is capable of transferring polynucleotides and/or infecting the pathogenic microorganism. In some embodiments, the intermediary microorganism can be an engineered bacteria, virus, or phage that contains the composition, system,(s) and/or component(s) thereof and/or CRISPR-Cas vectors and/or vector systems. The method can include administering an intermediary microorganism containing the composition, system,(s) and/or component(s) thereof and/or CRISPR-Cas vectors and/or vector systems to the subject to be treated. The intermediary microorganism can then produce the CRISPR-system and/or component thereof or transfer a composition, system, polynucleotide to the pathogenic organism. In embodiments, where the CRISPR-system and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In some embodiments, where the pathogenic microorganism inserts its genetic material into the host cell's genome (e.g. a virus), the composition, system can be designed such that it modifies the host cell's genome such that the viral DNA or cDNA cannot be replicated by the host cell's machinery into a functional virus. In some embodiments, where the pathogenic microorganism inserts its genetic material into the host cell's genome (e.g. a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In some embodiments, mtDNA mutations can be modified using a composition, system, described herein. In some embodiments, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In some embodiments, the mtDNA of a subject can be modified in vivo or ex vivo. In some embodiments, where the mtDNA is modified ex vivo, after modification the cells containing the modified mitochondria can be administered back to the subject. In some embodiments, the composition, system, or component thereof can be capable of correcting an mtDNA mutation, or a combination thereof.

In some embodiments, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In some embodiments, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In some embodiments, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In some embodiments, the microbe population of a microbiome in a subject can be modified using a composition, system, and/or component thereof described herein. In some embodiments, the composition, system, and/or component thereof can be used to identify and select one or more cell types in the microbiome and remove them from the microbiome population. Exemplary methods of selecting cells using a composition, system, and/or component thereof are described elsewhere herein. In this way the make-up or microorganism profile of the microbiome can be altered. In some embodiments, the alteration causes a change from a diseased microbiome composition to a healthy microbiome composition. In this way the ratio of one type or species of microorganism to another can be modified, such as going from a diseased ratio to a healthy ratio. In some embodiments, the cells selected are pathogenic microorganisms.

In some embodiments, the compositions, systems, described herein can be used to modify a polynucleotide in a microorganism of a microbiome in a subject. In some embodiments, the microorganism is a pathogenic microorganism. In some embodiments, the microorganism is a commensal and non-pathogenic microorganism. Methods of modifying polynucleotides in a cell in the subject are described elsewhere herein and can be applied to these embodiments.

### Adoptive Therapy

The compositions, systems, and components thereof described herein can be used to modify cells for an adoptive cell therapy. In an aspect of the invention, methods and compositions which involve editing a target nucleic acid sequence, or modulating expression of a target nucleic acid sequence, and applications thereof in connection with cancer immunotherapy are comprehended by adapting the composition, system, of the present invention.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gp1OO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGa1p(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGa1p(1-4)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pm1/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3 ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM 006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS)) (SEQ ID NO: 42). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 43) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 43) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO: 42). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CART cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, Siglec 7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In certain embodiments, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L 1 and/or CTLA4); and (3) knock-out or knock-down expression of one or more MEW constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-WIC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled β2-microglobulin (β2m) into WIC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Treating and Preventing Diseases Using RNA Editing

In some embodiments, the disease, disorder, and/condition or symptom thereof can be treated or prevented using a composition or system described herein. In some embodiments, the composition, system, described herein is a nucleic acid editing system. In some embodiments, treatment or prevention using a composition or system described herein can have the advantage of less immunogenicity than a nucleic acid editing composition, system, and is not as hindered by limitations on viral vector packaging size. Further, as the effect is transient, the effect can be better controlled over time and can potentially be reversible. Thus, they pose less risk of causing permeant detrimental effects than nucleic acid editing-based preventatives and treatments.

Any disease involving a dysfunctional nucleic acid molecule, where the dysfunction is the result of a mutation in the nucleic acid sequence can be treated or prevented by modifying its sequence using a composition, system, capable of nucleic acid modification described elsewhere herein. In some embodiments, the disease that can be treated or prevented using a composition, system, capable of RNA modification can be one or more of those listed in Tables 3-4, or a combination thereof. In some embodiments, the coding sequence for the gene involved in the disease is greater than the packaging capacity of a viral vector system, particularly an AAV vector system.

The potential for RNA editing has now been demonstrated in vitro and in vivo for pathogenic mutations in genes related to cystic fibrosis, Duchenne's muscular dystrophy, Hurler's syndrome, and Ornithine transcarbamylase (OTC) deficiency, among others. See e.g. Katrekar et al. Nat. Methods. 2019. 16:239-242; Montieel-Gonzalez et la. 2013. PNAS USA. 110: 18285-18290; Sinnamon et al. PNAS USA 2017; Wettengel et al. Curr. Gene Ther. 2018, 18:31-39; Qu et al. BioRxiv. 2019., 605972; and Fry et al. 2020. Int. J. Mol. Sci. 12:777, which are incorporated by reference as if expressed in their entirety here and the teachings of which can be adapted in view of the description herein to the CRISPR-Cas Systems described herein.

In some embodiments, the disease is an inherited retinal degeneration disease. In some embodiments, gene whose transcript can be modified using a composition, system, described herein capable of nucleic acid modification that is associated with inherited retinal degeneration and whose coding sequence is too large for packaging in a single AAV can be ABC4, USH2A, CEP290, MYO7A, EYS, and CDH23.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the invention provides a method of generating a model eukaryotic cell that can include one or more a mutated disease genes and/or infectious microorganisms. In some embodiments, a disease gene is any gene associated an increase in the risk of having or developing a disease. In some embodiments, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a guide sequence optionally linked to a tracr mate sequence, a tracr sequence, one or more Cas effectors, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more CRISPR-Cas effectors complexed with (1) one or more guide sequences that is/are hybridized to the target sequence(s) within the target polynucleotide(s), and optionally (2) the tracr mate sequence(s) that is/are hybridized to the tracr sequence(s), thereby generating a model eukaryotic cell comprising one or more mutated disease gene(s). Thus, in some embodiments the composition, system, contains nucleic acid molecules for and drives expression of one or more of: a Cas effector, a guide sequence linked to a tracr mate sequence, and a tracr sequence and/or a Homologous Recombination template and/or a stabilizing ligand if the Cas effector has a destabilization domain. In some embodiments, said cleavage comprises cleaving one or two strands at the location of the target sequence by the Cas effector(s). In some embodiments, nicking comprises nicking one or two strands at the location of the target sequence by the Cas effector(s). In some embodiments, said cleavage or nicking results in modified transcription of a target polynucleotide. In some embodiments, modification results in decreased transcription of the target polynucleotide. In some embodiments, the method further comprises repairing said cleaved or nicked target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

The disease modeled can be any disease with a genetic or epigenetic component. In some embodiments, the disease modeled can be any as discussed elsewhere herein, including but not limited to any as set forth in Tables 3 and 4 herein.

In situ Disease Detection

The compositions, systems, and/or components thereof can be used for diagnostic methods of detection such as in CASFISH (see e.g. Deng et al. 2015. PNAS USA 112(38): 11870-11875), CRISPR-Live FISH (see e.g. Wang et al. 2020. Science; 365(6459):1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g. Deng et al. 2015. PNAS USA 112(38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), ç, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In some embodiments, the composition, system, or component thereof can be used in a detection method, such as an in situ detection method described herein. In some embodiments, the composition, system, or component thereof can include a catalytically inactivate Cas effector described herein and use this system in detection methods such as fluorescence in situ hybridization (FISH) or any other described herein. In some embodiments, the inactivated Cas effector, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dCas effector or system thereof can be used to visualize both repetitive sequences and individual genes in the human genome. Such new applications of labelled dCas effector and compositions, systems, thereof can be important in imaging cells and studying the functional nuclear architecture, especially in cases with a small nucleus volume or complex 3-D structures.

### Cell Selection

In some embodiments, the compositions, systems, and/or components thereof described herein can be used in a method to screen and/or select cells. In some embodiments, composition, system-based screening/selection method can be used to identify diseased cells in a cell population. In some embodiments, selection of the cells results in a modification in the cells such that the selected cells die. In this way, diseased cells can be identified, and removed from the healthy cell population. In some embodiments, the diseased cells can be a cancer cell, pre-cancerous cell, a virus or other pathogenic organism infected cells, or otherwise abnormal cell. In some embodiments, the modification can impart another detectable change in the cells to be selected (e.g. a functional change and/or genomic barcode) that facilitates selection of the desired cells. In some embodiments a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In some embodiments, a method of selecting one or more cell(s) containing a polynucleotide modification can include: introducing one or more composition, system,(s) and/or components thereof, and/or vectors or vector systems into the cell(s), wherein the composition, system,(s) and/or components thereof, and/or vectors or vector systems contains and/or is capable of expressing one or more of: a Cas effector, a guide sequence optionally linked to a tracr mate sequence, a tracr sequence, and an editing template; wherein, for example that which is being expressed is within and expressed in vivo by the composition, system, vector or vector system and/or the editing template comprises the one or more mutations that abolish Cas effector cleavage; allowing homologous recombination of the editing template with the target polynucleotide in the cell(s) to be selected; allowing a composition, system, or complex to bind to a target polynucleotide to effect cleavage of the target polynucleotide within said gene, wherein the AAV-CAST complex comprises the Cas effector complexed with (1) the guide sequence that is hybridized to the target sequence within the target polynucleotide, and (2) the tracr mate sequence that is hybridized to the tracr sequence, wherein binding of the complex to the target polynucleotide induces cell death or imparts some other detectable change to the cell, thereby allowing one or more cell(s) in which one or more mutations have been introduced to be selected. In some embodiments, the cell to be selected may be a eukaryotic cell. In some embodiments, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop CRISPR-Cas-based and non-CRISPR-Cas-based biologically active agents, such as small molecule therapeutics. Thus, described herein are methods for developing a biologically active agent that modulates a cell function and/or signaling event associated with a disease and/or disease gene. In some embodiments, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In some embodiments, the diseased cell is a model cell described elsewhere herein. In some embodiments, the diseased cell is a diseased cell isolated from a subject in need of treatment. In some embodiments, the test compound is a small molecule agent. In some embodiments, test compound is a small molecule agent. In some embodiments, the test compound is a biologic molecule agent.

In some embodiments, the method involves developing a therapeutic based on the composition, system, described herein. In particular embodiments, the therapeutic comprises a Cas effector and/or a guide RNA capable of hybridizing to a target sequence of interest. In particular embodiments, the therapeutic is a vector or vector system that can contain a) a first regulatory element operably linked to a nucleotide sequence encoding the Cas effector protein(s); and b) a second regulatory element operably linked to one or more nucleotide sequences encoding one or more nucleic acid molecules comprising a guide RNA comprising a guide sequence, a direct repeat sequence; wherein components (a) and (b) are located on same or different vectors. In particular embodiments, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In some embodiments, the complex can include the Cas effector protein(s) as described herein, guide RNA comprising the guide sequence, and a direct repeat sequence. In any such compositions, the delivery system can be a yeast system, a lipofection system, a microinjection system, a biolistic system, virosomes, liposomes, immunoliposomes, polycations, lipid:nucleic acid conjugates or artificial virions, or any other system as described herein. In particular embodiments, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, and optionally validating one or more of the (sub)selected target sites for an individual subject, optionally designing one or more gRNA recognizing one or more of said (sub)selected target sites.

In some embodiments, the method for developing or designing a gRNA for use in a composition, system, optionally a composition, system, based therapy or therapeutic, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, thee method for developing or designing a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments the method for developing or designing a gRNA for use in a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, the method for developing or designing a composition, system, such as a composition, system, based therapy or therapeutic, optionally in a population; or for developing or designing a gRNA for use in a composition, system, optionally a composition, system, based therapy or therapeutic, optionally in a population, can include: selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of CRISP-Cas systems prepared is based (at least in part) on the size of a target population.

In certain embodiments, off-target candidates/off-targets, PAM restrictiveness, target cleavage efficiency, or effector protein specificity is identified or determined using a sequencing-based double-strand break (DSB) detection assay, such as described herein elsewhere. In certain embodiments, off-target candidates/off-targets are identified or determined using a sequencing-based double-strand break (DSB) detection assay, such as described herein elsewhere. In certain embodiments, off-targets, or off target candidates have at least 1, preferably 1-3, mismatches or (distal) PAM mismatches, such as 1 or more, such as 1, 2, 3, or more (distal) PAM mismatches. In certain embodiments, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the guide sequence of the gRNA is 100% complementary to the target site, i.e. does not comprise any mismatch with the target site. It will be further understood that “recognition” of an (off-)target site by a gRNA presupposes composition, system, functionality, i.e. an (off-)target site is only recognized by a gRNA if binding of the gRNA to the (off-)target site leads to composition, system, activity (such as induction of single or double strand DNA cleavage, transcriptional modulation, etc.).

In certain embodiments, the target sites having minimal sequence variation across a population are characterized by absence of sequence variation in at least 99%, preferably at least 99.9%, more preferably at least 99.99% of the population. In certain embodiments, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In certain embodiments, said population comprises at least 1000 individuals, such as at least 5000 individuals, such as at least 10000 individuals, such as at least 50000 individuals.

In certain embodiments, the off-target sites are characterized by at least one mismatch between the off-target site and the gRNA. In certain embodiments, the off-target sites are characterized by at most five, preferably at most four, more preferably at most three mismatches between the off-target site and the gRNA. In certain embodiments, the off-target sites are characterized by at least one mismatch between the off-target site and the gRNA and by at most five, preferably at most four, more preferably at most three mismatches between the off-target site and the gRNA.

In certain embodiments, said minimal number of off-target sites across said population is determined for high-frequency haplotypes in said population. In certain embodiments, said minimal number of off-target sites across said population is determined for high-frequency haplotypes of the off-target site locus in said population. In certain embodiments, said minimal number of off-target sites across said population is determined for high-frequency haplotypes of the target site locus in said population. In certain embodiments, the high-frequency haplotypes are characterized by occurrence in at least 0.1% of the population.

In certain embodiments, the number of (sub)selected target sites needed to treat a population is estimated based on based low frequency sequence variation, such as low frequency sequence variation captured in large scale sequencing datasets. In certain embodiments, the number of (sub)selected target sites needed to treat a population of a given size is estimated.

In certain embodiments, the method further comprises obtaining genome sequencing data of a subject to be treated; and treating the subject with a composition, system, selected from the set of compositions, systems, wherein the composition, system, selected is based (at least in part) on the genome sequencing data of the individual. In certain embodiments, the ((sub)selected) target is validated by genome sequencing, preferably whole genome sequencing.

In certain embodiments, target sequences or loci as described herein are (further) selected based on optimization of one or more parameters, such as PAM type (natural or modified), PAM nucleotide content, PAM length, target sequence length, PAM restrictiveness, target cleavage efficiency, and target sequence position within a gene, a locus or other genomic region. Methods of optimization are discussed in greater detail elsewhere herein.

In certain embodiments, target sequences or loci as described herein are (further) selected based on optimization of one or more of target loci location, target length, target specificity, and PAM characteristics. As used herein, PAM characteristics may comprise for instance PAM sequence, PAM length, and/or PAM GC contents. In certain embodiments, optimizing PAM characteristics comprises optimizing nucleotide content of a PAM. In certain embodiments, optimizing nucleotide content of PAM is selecting a PAM with a motif that maximizes abundance in the one or more target loci, minimizes mutation frequency, or both. Minimizing mutation frequency can for instance be achieved by selecting PAM sequences devoid of or having low or minimal CpG.

In certain embodiments, the effector protein for each composition, system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

The activity of the composition and/or system, such as CRISPR-Cas or CAST system-based therapy or therapeutics may involve target disruption, such as target mutation, such as leading to gene knockout. The activity of the composition and/or system, such as CRISPR-Cas or CAST system-based therapy or therapeutics may involve replacement of particular target sites, such as leading to target correction. CRISPR-Cas or CAST system-based therapy or therapeutics may involve removal of particular target sites, such as leading to target deletion. The activity of the composition and/or system, such as CRISPR-Cas or CAST system-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve CRISPR effector mutation (such as for instance generation of a catalytically inactive CRISPR effector) and/or functionalization (such as for instance fusion of the CRISPR effector with a heterologous functional domain, such as a transcriptional activator or repressor), as described herein elsewhere.

Accordingly, in an aspect, the invention relates to a method as described herein, comprising selection of one or more (therapeutic) target, selecting one or more functionality of the composition and/or system, and optimization of selected parameters or variables associated with the CRISPR-Cas or CAST system and/or its functionality. In a related aspect, the invention relates to a method as described herein, comprising (a) selecting one or more (therapeutic) target loci, (b) selecting one or more CRISPR-Cas system functionalities, (c) optionally selecting one or more modes of delivery, and preparing, developing, or designing a CRISPR-Cas system or CAST selected based on steps (a)-(c).

In certain embodiments, the functionality of the composition and/or system comprises genomic mutation. In certain embodiments, the functionality of the composition and/or system comprises single genomic mutation. In certain embodiments, the functionality of the composition and/or system functionality comprises multiple genomic mutation. In certain embodiments, the functionality of the composition and/or system comprises gene knockout. In certain embodiments, the functionality of the composition and/or system comprises single gene knockout. In certain embodiments, the functionality of the composition and/or system comprises multiple gene knockout. In certain embodiments, the functionality of the composition and/or system comprises gene correction. In certain embodiments, the functionality of the composition and/or system comprises single gene correction. In certain embodiments, the functionality of the composition and/or system comprises multiple gene correction. In certain embodiments, the functionality of the composition and/or system comprises genomic region correction. In certain embodiments, the functionality of the composition and/or system comprises single genomic region correction. In certain embodiments, the functionality of the composition and/or system comprises multiple genomic region correction. In certain embodiments, the functionality of the composition and/or system comprises gene deletion. In certain embodiments, the functionality of the composition and/or system comprises single gene deletion. In certain embodiments, the functionality of the composition and/or system comprises multiple gene deletion. In certain embodiments, the functionality of the composition and/or system comprises genomic region deletion. In certain embodiments, the functionality of the composition and/or system comprises single genomic region deletion. In certain embodiments, the functionality of the composition and/or system comprises multiple genomic region deletion. In certain embodiments, the functionality of the composition and/or system comprises modulation of gene or genomic region functionality. In certain embodiments, the functionality of the composition and/or system comprises modulation of single gene or genomic region functionality. In certain embodiments, the functionality of the composition and/or system comprises modulation of multiple gene or genomic region functionality. In certain embodiments, the functionality of the composition and/or system comprises gene or genomic region functionality, such as gene or genomic region activity. In certain embodiments, the functionality of the composition and/or system comprises single gene or genomic region functionality, such as gene or genomic region activity. In certain embodiments, the functionality of the composition and/or system comprises multiple gene or genomic region functionality, such as gene or genomic region activity. In certain embodiments, the functionality of the composition and/or system comprises modulation gene activity or accessibility optionally leading to transcriptional and/or epigenetic gene or genomic region activation or gene or genomic region silencing. In certain embodiments, the functionality of the composition and/or system comprises modulation single gene activity or accessibility optionally leading to transcriptional and/or epigenetic gene or genomic region activation or gene or genomic region silencing. In certain embodiments, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as CRISPR-Cas or CAST system-based therapy or therapeutic, specificity, efficacy, and/or safety. In certain embodiments, one or more of the following parameters or variables are taken into account, are selected, or are optimized in the methods of the invention as described herein: Cas protein allosteric interactions, Cas protein functional domains and functional domain interactions, CRISPR effector specificity, gRNA specificity, CRISPR-Cas complex specificity, PAM restrictiveness, PAM type (natural or modified), PAM nucleotide content, PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas or CAST complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas or CAST complex dose or titer, CRISPR effector protein size, CRISPR effector expression level, gRNA expression level, CRISPR-Cas or CAST complex expression level, CRISPR effector spatiotemporal expression, gRNA spatiotemporal expression, CRISPR-Cas or CAST complex spatiotemporal expression.

By means of example, and without limitation, parameter or variable optimization may be achieved as follows. CRISPR effector specificity may be optimized by selecting the most specific CRISPR effector. This may be achieved for instance by selecting the most specific CRISPR effector orthologue or by specific CRISPR effector mutations which increase specificity. gRNA specificity may be optimized by selecting the most specific gRNA. This can be achieved for instance by selecting gRNA having low homology, i.e. at least one or preferably more, such as at least 2, or preferably at least 3, mismatches to off-target sites. CRISPR-Cas complex specificity may be optimized by increasing CRISPR effector specificity and/or gRNA specificity as above. PAM restrictiveness may be optimized by selecting a CRISPR effector having to most restrictive PAM recognition. This can be achieved for instance by selecting a CRISPR effector orthologue having more restrictive PAM recognition or by specific CRISPR effector mutations which increase or alter PAM restrictiveness. PAM type may be optimized for instance by selecting the appropriate CRISPR effector, such as the appropriate CRISPR effector recognizing a desired PAM type. The CRISPR effector or PAM type may be naturally occurring or may for instance be optimized based on CRISPR effector mutants having an altered PAM recognition, or PAM recognition repertoire. PAM nucleotide content may for instance be optimized by selecting the appropriate CRISPR effector, such as the appropriate CRISPR effector recognizing a desired PAM nucleotide content. The CRISPR effector or PAM type may be naturally occurring or may for instance be optimized based on CRISPR effector mutants having an altered PAM recognition, or PAM recognition repertoire. PAM length may for instance be optimized by selecting the appropriate CRISPR effector, such as the appropriate CRISPR effector recognizing a desired PAM nucleotide length. The CRISPR effector or PAM type may be naturally occurring or may for instance be optimized based on CRISPR effector mutants having an altered PAM recognition, or PAM recognition repertoire.

Target length or target sequence length may for instance be optimized by selecting the appropriate CRISPR effector, such as the appropriate CRISPR effector recognizing a desired target or target sequence nucleotide length. Alternatively, or in addition, the target (sequence) length may be optimized by providing a target having a length deviating from the target (sequence) length typically associated with the CRISPR effector, such as the naturally occurring CRISPR effector. The CRISPR effector or target (sequence) length may be naturally occurring or may for instance be optimized based on CRISPR effector mutants having an altered target (sequence) length recognition, or target (sequence) length recognition repertoire. For instance, increasing or decreasing target (sequence) length may influence target recognition and/or off-target recognition. CRISPR effector activity may be optimized by selecting the most active CRISPR effector. This may be achieved for instance by selecting the most active CRISPR effector orthologue or by specific CRISPR effector mutations which increase activity. The ability of the CRISPR effector protein to access regions of high chromatin accessibility, may be optimized by selecting the appropriate CRISPR effector or mutant thereof, and can consider the size of the CRISPR effector, charge, or other dimensional variables etc. The degree of uniform CRISPR effector activity may be optimized by selecting the appropriate CRISPR effector or mutant thereof, and can consider CRISPR effector specificity and/or activity, PAM specificity, target length, mismatch tolerance, epigenetic tolerance, CRISPR effector and/or gRNA stability and/or half-life, CRISPR effector and/or gRNA immunogenicity and/or toxicity, etc. gRNA activity may be optimized by selecting the most active gRNA. In some embodiments, this can be achieved by increasing gRNA stability through RNA modification. CRISPR-Cas complex activity may be optimized by increasing CRISPR effector activity and/or gRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e. having low variability, such as polymorphisms, within a population).

In certain embodiments, optimizing target (sequence) length comprises selecting a target sequence within one or more target loci between 5 and 25 nucleotides. In certain embodiments, a target sequence is 20 nucleotides.

In certain embodiments, optimizing target specificity comprises selecting targets loci that minimize off-target candidates.

In some embodiments, the target site may be selected by minimization of off-target effects (e.g. off-targets qualified as having 1-5, 1-4, or preferably 1-3 mismatches compared to target and/or having one or more PAM mismatches, such as distal PAM mismatches), preferably also considering variability within a population. CRISPR effector stability may be optimized by selecting CRISPR effector having appropriate half-life, such as preferably a short half-life while still capable of maintaining sufficient activity. In some embodiments, this can be achieved by selecting an appropriate CRISPR effector orthologue having a specific half-life or by specific CRISPR effector mutations or modifications which affect half-life or stability, such as inclusion (e.g. fusion) of stabilizing or destabilizing domains or sequences. CRISPR effector mRNA stability may be optimized by increasing or decreasing CRISPR effector mRNA stability. In some embodiments, this can be achieved by increasing or decreasing CRISPR effector mRNA stability through mRNA modification. gRNA stability may be optimized by increasing or decreasing gRNA stability. In some embodiments, this can be achieved by increasing or decreasing gRNA stability through RNA modification. CRISPR-Cas or CAST complex stability may be optimized by increasing or decreasing CRISPR effector stability and/or gRNA stability as above. CRISPR effector protein or mRNA immunogenicity or toxicity may be optimized by decreasing CRISPR effector protein or mRNA immunogenicity or toxicity. In some embodiments, this can be achieved by mRNA or protein modifications. Similarly, in case of DNA based expression systems, DNA immunogenicity or toxicity may be decreased. gRNA immunogenicity or toxicity may be optimized by decreasing gRNA immunogenicity or toxicity. In some embodiments, this can be achieved by gRNA modifications. Similarly, in case of DNA based expression systems, DNA immunogenicity or toxicity may be decreased. CRISPR-Cas or CAST complex immunogenicity or toxicity may be optimized by decreasing CRISPR effector immunogenicity or toxicity and/or gRNA immunogenicity or toxicity as above, or by selecting the least immunogenic or toxic CRISPR effector/gRNA combination. Similarly, in case of DNA based expression systems, DNA immunogenicity or toxicity may be decreased. CRISPR effector protein or mRNA dose or titer may be optimized by selecting dosage or titer to minimize toxicity and/or maximize specificity and/or efficacy. gRNA dose or titer may be optimized by selecting dosage or titer to minimize toxicity and/or maximize specificity and/or efficacy. CRISPR-Cas or CAST complex dose or titer may be optimized by selecting dosage or titer to minimize toxicity and/or maximize specificity and/or efficacy. CRISPR effector protein size may be optimized by selecting minimal protein size to increase efficiency of delivery, in particular for virus mediated delivery. CRISPR effector, gRNA, or CRISPR-Cas or CAST complex expression level may be optimized by limiting (or extending) the duration of expression and/or limiting (or increasing) expression level. This may be achieved for instance by using self-inactivating compositions, systems, such as including a self-targeting (e.g. CRISPR effector targeting) gRNA, by using viral vectors having limited expression duration, by using appropriate promoters for low (or high) expression levels, by combining different delivery methods for individual CRISP-Cas or CAST system components, such as virus mediated delivery of CRISPR-effector encoding nucleic acid combined with non-virus mediated delivery of gRNA, or virus mediated delivery of gRNA combined with non-virus mediated delivery of CRISPR effector protein or mRNA. CRISPR effector, gRNA, or CRISPR-Cas or CAST complex spatiotemporal expression may be optimized by appropriate choice of conditional and/or inducible expression systems, including controllable CRISPR effector activity optionally a destabilized CRISPR effector and/or a split CRISPR effector, and/or cell- or tissue-specific expression systems.

In an aspect, the invention relates to a method as described herein, comprising selection of one or more (therapeutic) target, selecting the functionality of the composition and/or system, selecting CRISPR-Cas system mode of delivery, selecting CRISPR-Cas or CAST system delivery vehicle or expression system, and optimization of selected parameters or variables associated with the CRISPR-Cas system and/or its functionality, optionally wherein the parameters or variables are one or more selected from CRISPR effector specificity, gRNA specificity, CRISPR-Cas or CAST complex specificity, PAM restrictiveness, PAM type (natural or modified), PAM nucleotide content, PAM length, CRISPR effector activity, gRNA activity, CRISPR-Cas c or CAST complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas or CAST complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas or CAST complex dose or titer, CRISPR effector protein size, CRISPR effector expression level, gRNA or CAST expression level, CRISPR-Cas complex expression level, CRISPR effector spatiotemporal expression, gRNA spatiotemporal expression, CRISPR-Cas or CAST complex spatiotemporal expression.

In an aspect, the invention relates to a method as described herein, comprising selecting one or more (therapeutic) target, selecting one or more the functionality of the composition and/or system, selecting one or more CRISPR-Cas or CAST system mode of delivery, selecting one or more delivery vehicle or expression system, and optimization of selected parameters or variables associated with the CRISPR-Cas or CAST system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas or CAST complex specificity, PAM restrictiveness, PAM type (natural or modified), PAM nucleotide content, PAM length, wherein optimization of efficacy comprises optimizing one or more parameters or variables selected from CRISPR effector activity, gRNA activity, CRISPR-Cas or CAST complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas or CAST complex stability, CRISPR effector protein or mRNA immunogenicity or toxicity, gRNA immunogenicity or toxicity, CRISPR-Cas or CAST complex immunogenicity or toxicity, CRISPR effector protein or mRNA dose or titer, gRNA dose or titer, CRISPR-Cas or CAST complex dose or titer, CRISPR effector expression level, gRNA expression level, CRISPR-Cas or CAST complex expression level, CRISPR effector spatiotemporal expression, gRNA spatiotemporal expression, CRISPR-Cas or CAST complex spatiotemporal expression.

In an aspect, the invention relates to a method as described herein, comprising optionally selecting one or more (therapeutic) target, optionally selecting one or more the functionality of the composition and/or system, optionally selecting one or more mode of delivery, optionally selecting one or more delivery vehicle or expression system, and optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas or CAST complex specificity, PAM restrictiveness, PAM type (natural or modified), PAM nucleotide content, PAM length, wherein optimization of efficacy comprises optimizing one or more parameters or variables selected from CRISPR effector activity, gRNA activity, CRISPR-Cas or CAST complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas or CAST complex dose or titer, CRISPR effector expression level, gRNA expression level, CRISPR-Cas or CAST complex expression level, CRISPR effector spatiotemporal expression, gRNA spatiotemporal expression, CRISPR-Cas complex spatiotemporal expression.

In an aspect, the invention relates to a method as described herein, comprising optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas or CAST complex specificity, PAM restrictiveness, PAM type (natural or modified), PAM nucleotide content, PAM length, wherein optimization of efficacy comprises optimizing one or more parameters or variables selected from CRISPR effector activity, gRNA activity, CRISPR-Cas or CAST complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas or CAST complex immunogenicity or toxicity, CRISPR effector protein or mRNA dose or titer, gRNA dose or titer, CRISPR-Cas or CAST complex dose or titer, CRISPR effector expression level, gRNA expression level, CRISPR-Cas or CAST complex expression level, CRISPR effector spatiotemporal expression, gRNA spatiotemporal expression, CRISPR-Cas or CAST complex spatiotemporal expression.

It will be understood that the parameters or variables to be optimized as well as the nature of optimization may depend on the (therapeutic) target, the functionality of the composition and/or system, the system mode of delivery, and/or the CRISPR-Cas system delivery vehicle or expression system.

In an aspect, the invention relates to a method as described herein, comprising optimization of gRNA specificity at the population level. Preferably, said optimization of gRNA specificity comprises minimizing gRNA target site sequence variation across a population and/or minimizing gRNA off-target incidence across a population.

In some embodiments, optimization can result in selection of a CRISPR-Cas effector that is naturally occurring or is modified. In some embodiments, optimization can result in selection of a CRISPR-Cas effector that has nuclease, nickase, deaminase, transposase, and/or has one or more effector functionalities deactivated or eliminated. In some embodiments, optimizing a PAM specificity can include selecting a CRISPR-Cas effector with a modified PAM specificity. In some embodiments, optimizing can include selecting a CRISPR-Cas effector having a minimal size. In certain embodiments, optimizing effector protein stability comprises selecting an effector protein having a short half-life while maintaining sufficient activity, such as by selecting an appropriate CRISPR effector orthologue having a specific half-life or stability. In certain embodiments, optimizing immunogenicity or toxicity comprises minimizing effector protein immunogenicity or toxicity by protein modifications. In certain embodiments, optimizing functional specific comprises selecting a protein effector with reduced tolerance of mismatches and/or bulges between the guide RNA and one or more target loci.

In certain embodiments, optimizing efficacy comprises optimizing overall efficiency, epigenetic tolerance, or both. In certain embodiments, maximizing overall efficiency comprises selecting an effector protein with uniform enzyme activity across target loci with varying chromatin complexity, selecting an effector protein with enzyme activity limited to areas of open chromatin accessibility. In certain embodiments, chromatin accessibility is measured using one or more of ATAC-seq, or a DNA-proximity ligation assay. In certain embodiments, optimizing epigenetic tolerance comprises optimizing methylation tolerance, epigenetic mark competition, or both. In certain embodiments, optimizing methylation tolerance comprises selecting an effector protein that modify methylated DNA. In certain embodiments, optimizing epigenetic tolerance comprises selecting an effector protein unable to modify silenced regions of a chromosome, selecting an effector protein able to modify silenced regions of a chromosome, or selecting target loci not enriched for epigenetic markers

In certain embodiments, selecting an optimized guide RNA comprises optimizing gRNA stability, gRNA immunogenicity, or both, or other gRNA associated parameters or variables as described herein elsewhere.

In certain embodiments, optimizing gRNA stability and/or gRNA immunogenicity comprises RNA modification, or other gRNA associated parameters or variables as described herein elsewhere. In certain embodiments, the modification comprises removing 1-3 nucleotides form the 3′ end of a target complementarity region of the gRNA. In certain embodiments, modification comprises an extended gRNA and/or trans RNA/DNA element that create stable structures in the gRNA that compete with gRNA base pairing at a target of off-target loci, or extended complimentary nucleotides between the gRNA and target sequence, or both.

In certain embodiments, the mode of delivery comprises delivering gRNA and/or CRISPR effector protein, delivering gRNA and/or CRISPR effector mRNA, or delivery gRNA and/or CRISPR effector as a DNA based expression system. In certain embodiments, the mode of delivery further comprises selecting a delivery vehicle and/or expression systems from the group consisting of liposomes, lipid particles, nanoparticles, biolistics, or viral-based expression/delivery systems. In certain embodiments, expression is spatiotemporal expression is optimized by choice of conditional and/or inducible expression systems, including controllable CRISPR effector activity optionally a destabilized CRISPR effector and/or a split CRISPR effector, and/or cell- or tissue-specific expression system.

The methods as described herein may further involve selection of the mode of delivery. In certain embodiments, gRNA (and tracr, if and where needed, optionally provided as a sgRNA) and/or CRISPR effector protein are or are to be delivered. In certain embodiments, gRNA (and tracr, if and where needed, optionally provided as a sgRNA) and/or CRISPR effector mRNA are or are to be delivered. In certain embodiments, gRNA (and tracr, if and where needed, optionally provided as a sgRNA) and/or CRISPR effector provided in a DNA-based expression system are or are to be delivered. In certain embodiments, delivery of the individual system components comprises a combination of the above modes of delivery. In certain embodiments, delivery comprises delivering gRNA and/or CRISPR effector protein, delivering gRNA and/or CRISPR effector mRNA, or delivering gRNA and/or CRISPR effector as a DNA based expression system.

The methods as described herein may further involve selection of the CRISPR-Cas system delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g. adenoviral, AAV, lentiviral), etc. the skilled person will understand that selection of the mode of delivery, as well as delivery vehicle or expression system may depend on for instance the cell or tissues to be targeted. In certain embodiments, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a Cas (e.g. Cas9 and/or Cas12) nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels are needed to gain a clinically relevant response. In some embodiments, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state.

NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a CRISPR-Cas based therapeutic as described in greater detail elsewhere herein.

CRISPR-Cas- or CAST-based polynucleotide modification application can include combinations of proteins, small RNA molecules, and/or repair templates, and can make, in some embodiments, delivery of these multiple parts substantially more challenging than, for example, traditional small molecule therapeutics. Two main strategies for delivery of compositions, systems, and components thereof have been developed: ex vivo and in vivo. In some embodiments of ex vivo treatments, diseased cells are removed from a subject, edited and then transplanted back into the patient. In other embodiments, cells from a healthy allogeneic donor are collected, modified using a CRISPR-Cas system or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In some embodiments, such as those where viral vector systems are used to generate viral particles to deliver the CRISPR-Cas system and/or component thereof to a cell, the total cargo size of the CRISPR-Cas system and/or component thereof should be considered as vector systems can have limits on the size of a polynucleotide that can be expressed therefrom and/or packaged into cargo inside of a viral particle. In some embodiments, the tropism of a vector system, such as a viral vector system, should be considered as it can impact the cell type to which the CRISPR-Cas system or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In some embodiments, it can be important to considered the immunogenicity of the system or component thereof. In embodiments, where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the CRISPR enzyme in the host species (human or other species).

### Kits

In another aspect, the present disclosure provides kit and kit of parts. The terms “kit of parts” and “kit” as used throughout this specification refer to a product containing components necessary for carrying out the specified methods (e.g., methods for detecting, quantifying or isolating immune cells as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridization probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

The present application also provides aspects and embodiments as set forth in the following numbered Statements:

Statement 1. An engineered system, the system comprising: a. one or more CRISPR-associated Tn7 transposases; b. one or more Type I-B Cas proteins; and c. a guide molecule capable of complexing with the Type I-B Cas protein and directing binding of the guide-Cas protein complex to a target polynucleotide.

Statement 2. The system of Statement 1, wherein the one or more CRISPR-associated Tn7 transposases comprise TnsA, TnsB, TnsC, and/or TniQ.

Statement 3. The system of any one of proceeding Statements, wherein the Tn7 transposases comprise TnsA, TnsB, TnsC, a first TniQ and a second TniQ, wherein the first and second TniQ are different.

Statement 4. The system of any one of proceeding Statements, wherein the Tn7 transposases comprise comprises TnsA, TnsB, TnsC, and a TniQ.

Statement 5. The system of Statement 4, wherein the TniQ comprises a DNA-binding domain.

Statement 6. The system of Statement 5, wherein the DNA-binding domain is at C-terminus of the TniQ.

Statement 7. The system of any one of proceeding Statements, wherein the one or more CRISPR-associated Tn7 transposases comprise TnsA, TnsB, TnsC, and/or TnsD.

Statement 8. The system of Statement 7, wherein the Tn7 transposases comprise TnsA, TnsB, TnsC, a first TnsD and a second TnsD, wherein the first and second TnsD are different.

Statement 9. The system of Statement 7, wherein the Tn7 transposase comprises TnsA, TnsB, TnsC and a TnsD.

Statement 10. The system of Statement 9, wherein the TnsD comprises a DNA-binding domain.

Statement 11. The system of Statement 9 or 10, wherein the TnsA and TnsB are comprised in a single protein.

Statement 12. The system of Statement 9, 10, or 11, wherein the Tn7 transposase comprises: a. TnsA, TnsB, TnsC and TnsD1; b. TnsA, TnsB, TnsC and TnsD2; or c. TnsA, TnsB, TnsC, TnsD, and TnsD2.

Statement 13. The system of any one of proceeding Statements, wherein the one or more Type I-B Cas proteins comprises Cas5, Cas6, Cas7, and/or Cas 8.

Statement 14. The system of any one of proceeding Statements, wherein the one or more Type I-B Cas proteins comprises Cas 8b.

Statement 15. The system of Statement 14, wherein the Cas8b is Cas8b3.

Statement 16. The system of any one of proceeding Statements, wherein the one or more CRISPR-associated Tn7 transposases and/or the one or more Type I-B Cas proteins are from or originated from Anabaena variabilis.

Statement 17. The system of any one of proceeding Statements, wherein the one or more Type I-B Cas proteins lacks nuclease activity.

Statement 18. The system of any one of proceeding Statements, further comprising a donor polynucleotide.

Statement 19. The system of Statement 18, wherein the donor polynucleotide comprises a polynucleotide insert, a left element sequence, and a right element sequence.

Statement 20. The system of Statement 18 or 19, wherein the donor polynucleotide: a. introduces one or more mutations to the target polynucleotide; b. corrects a premature stop codon in the target polynucleotide; c. disrupts a splicing site; d. restores a splicing site; or e. a combination thereof.

Statement 21. The system of Statement 20, wherein the one or more mutations introduced by the donor polynucleotide comprises substitutions, deletions, insertions, or a combination thereof.

Statement 22. The system of Statement 20 or 21, wherein the one or more mutations causes a shift in an open reading frame on the target polynucleotide.

Statement 23. The system of any one of Statements 18-22, wherein the donor polynucleotide is between 100 bases and 30 kb in length.

Statement 24. The system of any one of proceeding Statements, wherein the target polynucleotide comprises a protospacer adjacent motif (PAM).

Statement 25. The system of Statement 24, wherein the PAM is AT or ATG.

Statement 26. The system of Statement 25, wherein the donor polynucleotide is inserted to the target polynucleotide at a site no more than 80, no more 90, no more 100, no more 200, or no more 300 bp 3′ of the PAM.

Statement 27. The system of Statement 25 or 26, wherein the donor polynucleotide is inserted to the target polynucleotide at a site no more than 80, no more 90, no more 100, no more 200, or no more 300 bp 5′ of the PAM.

Statement 28. The system of any one of proceeding Statements, wherein the target polynucleotide is linear, circular, or genomic DNA.

Statement 29. The system of any one of proceeding Statements, wherein the one or more Tn7 transposases is derived from a first species and the one or more Type I-B Cas proteins is derived from a second species different from the first species.

Statement 30. The system of Statement 29, comprising a first TnsD1 derived from the first species and a second TnsD2 derived from the second species.

Statement 31. The system of any one of proceeding Statements, wherein the one or more Tn7 transposases comprises a transposase with activity of TnsA and TnsB.

Statement 32. The system of any one of proceeding Statements, further comprising a targeting moiety.

Statement 33. The system of any one of proceeding Statements, which comprises a plurality of guide molecules capable of directing binding of the guide-Cas protein complex to one or more target polynucleotides.

Statement 34. A system comprising one or more polynucleotides encoding: a. one or more CRISPR-associated Tn7 transposases; b. one or more Type I-B Cas proteins; and c. a guide molecule capable of complexing with the Type I-B Cas protein and directing binding of the guide-Cas protein complex to a target polynucleotide.

Statement 35. The system of Statement 34, further comprising a donor polynucleotide.

Statement 36. The system of Statement 35, wherein the donor polynucleotide comprises a polynucleotide insert, a left element sequence, and a right element sequence.

Statement 37. The system of any one of Statements 34-36, comprising one or more polynucleotides or encoded products of the polynucleotides in one or more loci in Table 5.

Statement 38. The system of any one of Statements 34-37, wherein the one or more polynucleotides encode components (a)-(c) of any one of Statements 1-33.

Statement 39. The system of any one of Statements 34-38, wherein the one or more Type I-B Cas proteins comprises Cas5, Cas6, Cas7, and/or Cas 8.

Statement 40. The system of any one of Statements 34-39, wherein the system comprises a first polynucleotide encoding a first Cas6 and a second polynucleotide encoding a second Cas6.

Statement 41. A vector comprising the one or more polynucleotides of any one of Statements 34-40.

Statement 42. A engineered cell comprising the system of any one of Statements 1-40, or the vector of Statement 41.

Statement 43. The engineered cell of Statement 42, wherein the cell produces and/or secretes an endogenous or non-endogenous biological product or chemical compound.

Statement 44. The engineered cell of Statement 43, wherein the biological product is a protein or an RNA.

Statement 45. A cell line comprising the engineered cell of any one of Statements 42-44 and progeny thereof.

Statement 46. A plant or animal model comprising the engineered cell of any one of Statements 42-44 and progeny thereof.

Statement 47. A composition comprising the engineered cell of any one of Statements 42-44.

Statement 48. The composition of Statement 47, formulated for use as a therapeutic.

Statement 49. A biological product or chemical compound produced by the engineered cell of any one of Statements 42-44.

Statement 50. An engineered cell or progeny thereof, the cell being engineered by use of the system of any one of Statements 1-40.

Statement 51. The cell or progeny thereof of Statement 50 that is isolated.

Statement 52. The cell or progeny thereof of Statement 50 or 51 that is further used as a therapeutic.

Statement 53. The cell or progeny thereof of Statement 50, 51, or 52 from which a product is isolated.

Statement 54. A product produced by the cell or progeny thereof of Statement 50, 51, or 52.

Statement 55. The product of Statement 54, wherein the product is a protein or an RNA.

Statement 56. The protein of Statement 55, wherein the protein comprises a mutation.

Statement 57. A pharmaceutical composition for treatment of a disease or disorder, comprising the cell or progeny thereof of Statement 50, 51, or 52.

Statement 58. The pharmaceutical composition according to Statement 57, wherein the treatment results in genetic changes in one or more cells.

Statement 59. The pharmaceutical composition according to Statement 57 or 58, wherein the treatment results in correction of one or more defective genotypes.

Statement 60. The pharmaceutical composition according to Statement 57, 58, or 59, wherein the treatment results in improved phenotype.

Statement 61. The cell or progeny thereof of Statement 50, 51, or 52, wherein the cell comprises a mutation in a protein expressed from a gene comprising the target sequence.

Statement 62. The cell or progeny thereof of Statement 61, wherein the cell comprises deletion of a genomic region comprising the target sequence.

Statement 63. The cell or progeny thereof of any one of Statements 61-62, wherein the cell comprises integration of an exogenous sequence by homology-directed repair.

Statement 64. The cell or progeny thereof of any one of Statements 61-63, wherein the cell comprises decreased transcription of a gene associated with the target sequence.

Statement 65. The or progeny thereof of any one of Statements 61-64, wherein the cell comprises increased transcription of a gene associated with the target sequence.

Statement 66. The product of Statement 49, wherein the product is a mutated protein or product provided by a template.

Statement 67. A method of inserting a donor polynucleotide into a target polynucleotide in a cell, the method comprises introducing to the cell: a. one or more CRISPR-associated Tn7 transposases or functional fragments thereof; b. one or more Type I-B Cas proteins; and c. a guide molecule capable of complexing with the Type I-B Cas protein and directing binding of the guide-Cas protein complex to a target polynucleotide.

Statement 68. The method of Statement 67, wherein the donor polynucleotide: a. introduces one or more mutations to the target polynucleotide; b. corrects a premature stop codon in the target polynucleotide; c. disrupts a splicing site; d. restores a splicing site; or e. a combination thereof.

69. The method of Statement 68, wherein the one or more mutations introduced by the donor polynucleotide comprises substitutions, deletions, insertions, or a combination thereof.

Statement 70. The method of Statement 68 or 69, wherein the one or more mutations causes a shift in an open reading frame on the target polynucleotide.

Statement 71. The method of any one of Statements 67-70, wherein the donor polynucleotide is between 100 bases and 30 kb in length.

Statement 72. The method of any one of Statements 67-71, wherein one or more of components (a), (b), and (c) is expressed from a nucleic acid operably linked to a regulatory sequence.

Statement 73. The method of any one of Statements 67-72, wherein one or more of components (a), (b), and (c) is introduced in a particle.

Statement 74. The method of any one of Statements 67-73, wherein the particle comprises a ribonucleoprotein (RNP).

Statement 75. The method of any one of Statements 67-74, wherein the cell is a prokaryotic cell.

Statement 76. The method of any one of Statements 67-74, wherein the cell is a eukaryotic cell.

Statement 77. The method of any one of Statements 67-74, wherein the cell is a mammalian cell, a cell of a non-human primate, or a human cell.

Statement 78. The method of any one of Statements 67-74, wherein the cell is a plant cell.

Statement 79. The method of any one of Statements 67-78, wherein insertion of the donor polynucleotide into the target polynucleotide in the cell results in: a cell or population of cells comprising altered expression levels of one or more gene products; a cell or population of cells that produces and/or secrete an endogenous or non-endogenous biological product or chemical compound.

Statement 80. The method of any one of Statements 67-79, wherein the donor polynucleotide is inserted to a site no more than 80, no more 90, no more 100, no more 200, or no more 300 bp 3′ of a PAM on the target polynucleotide.

Statement 81. The method of any one of Statements 67-79, wherein the donor polynucleotide is inserted to a site no more than 80, no more 90, no more 100, no more 200, or no more 300 bp 5′ of a PAM on the target polynucleotide.

Statement 82. The method of any one of Statements 67-81, wherein the target polynucleotide is linear, circular, or genomic DNA.

### EXAMPLES

### Example 1—Exemplary CAST Systems

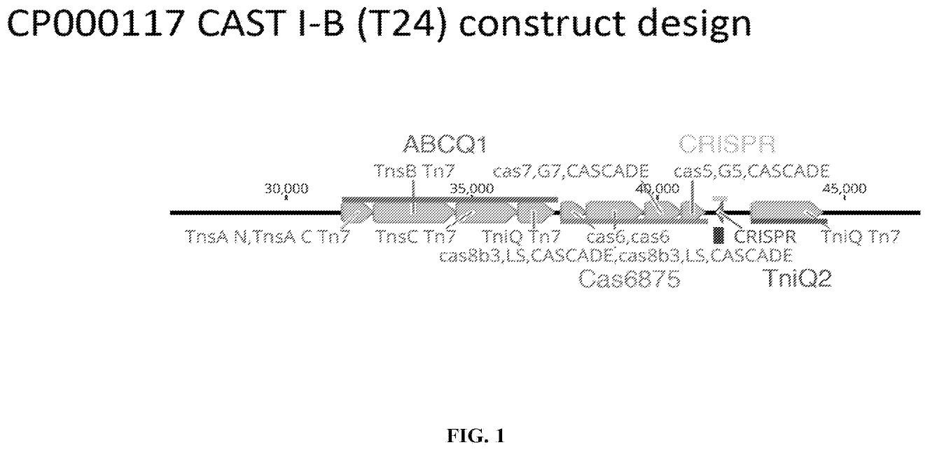

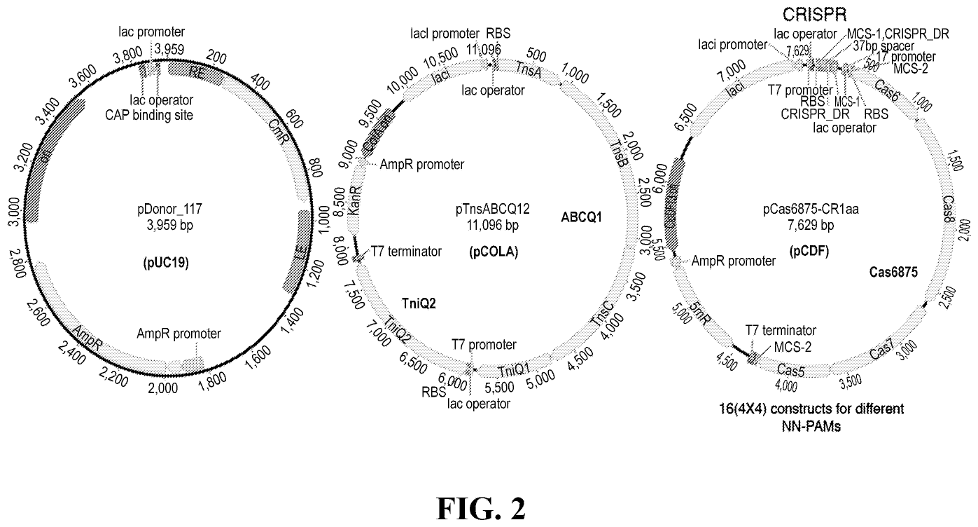

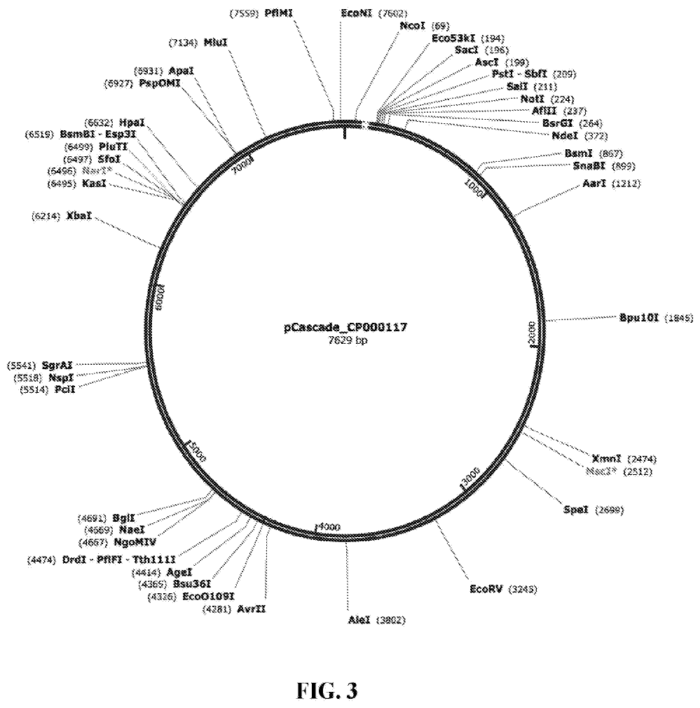

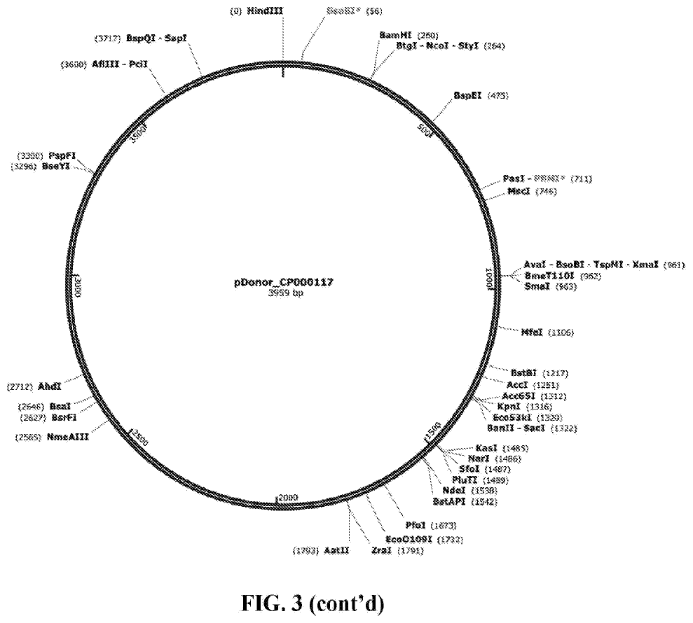

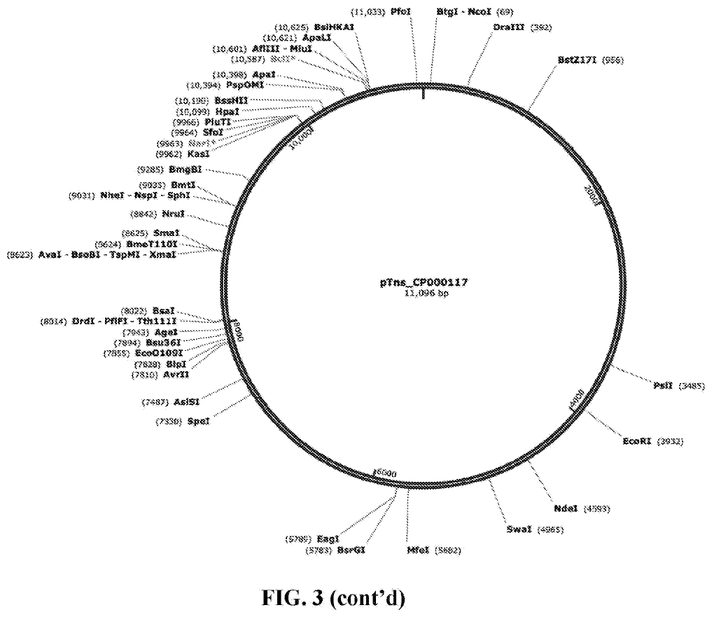

A construct of an exemplary Type I-B CAST system from locus CP000117 of Anabaena variabilis ATCC 29413 is shown in FIG. 1. The plasmids for expressing the CAST system are shown in FIG. 2. Detailed maps of the plasmids are shown in FIG. 3. The Sequences of the plasmids are shown in Table 5 below.

TABLE 6

| Plasmids | Sequences |
|---|---|
| pCascade_CP000117 | GGGGAATTGTGAGCGGATAACAATTCCCCTGTAGAAATAATTTTGTTTAACTTTAATAAGGAGATATACCATGGT |
| | GCTTTAACATTAGATGTCGTTAGGCGTTGAGCANNNNNNNNNNNNNNNNNNNNNNNNNNNNNNNNNNNNNGTG |
| | CTTTAACATTAGATGTCGTTAGGCGTTGAGCAGATCCGAATTCGAGCTCGGCGCGCCTGCAGGTCGACAAGCTTG |
| | CGGCCGCATAATGCTTAAGTCGAACAGAAAGTAATCGTATTGTACACGGCCGCATAATCGAAATTAATACGACTC |
| | ACTATAGGGGAATTGTGAGCGGATAACAATTCCCCATCTTAGTATATTAGTTAAGTATAAGAAGGAGATATACAT |
| | atgattattgatagtaacttagttacgcagcttccctcagaatcatcttcaaagccaagagagcttttaatgtct |
| | cagtttccatatatggatttaacttttagccttacgggtgagacattacctttcgaccacggctatgaattattttc |
| | tgcgatcgcccactttgaacccaaactacatacattagacacgttgggaattcagaccattgcaggtattcccaagg |
| | atggtgtaattaatctgacccaaaattctaggcttcgtattcggataccagtaaatcaggtacgtttggtttatcca |
| | ttggcgggtaagtcactgagaattggcaagcatacaattcgtttgggtattcctgacatctgtttgcttcaacctgc |
| | ggagaaactgcgatcgcgcattgtggtaataagaggctatgaagaacctgaaagttttttagtcgtagcgcagcg |
| | tcagctagaacaactgggtatccaagcaattgctagcattcccaccaaagccaatggcaaaccaatacgtaggacta |
| | tcaaaattaagcgatttacagtcgtgggttttggattagaagttataaatttaagcgatgaagactcactgaggttg |
| | caaatacatggtgtaggaggaaagcaaaagatgggttgtggactcttcatgcctattaaggaaaggcaatagcaatt |
| | actcgaaagcagatgatgaagagagcagataaccccaaaattcagcttaatctcggcgatcctagtatgactttatt |
| | gcatcgtgctggggttgcaggactttggatgacgctcaaaaaactggaaaaaatctatccaacacctgctgaacgta |
| | ttggtaatttaacttggttgctaacccctcactatatcaatcttgattgggaaggacaggatttcacagttttagac |
| | tggttgctgaagcaatcatttcaaatcagcgatgagggtttaatttccctgacagggttagattctcaaaacatgga |
| | tattcaaaatcaaataattatccatcaaggtatcacagccacttttacacaacataataaattttttaagtctgcgg |
| | gtcaacaatctaagcaactaattattgatggtattcaggttagggttgattataagaaagctgcttcctatgctcat |
| | caacattttgctaaacatttatgtgaccagtacggacaattgctaaaagagccaattggtatcaggggatggctcta |
| | tccaggagcagtagttcgccattatgcttttaaagaacaaacagcatttgaagaaaaagcagaatatgctctagcgt |
| | tgttgttcgctcctatggcttgtcaatattttgtgctgcgatcgcacaccgaaccatcacatacaaactatgttttg |
| | gttattcctgaagtaattgatttagaactttatgcacattttttgtggtgtttgggtaacttagattataaacattt |
| | tcacgtatctagcttaggagatgctggattaaaatttcttaattataaaacaacacaaatgcctacttccaattttt |
| | taaaacgatgtcaaataatcttttttagtacaaaagcttggtcagaacagcaaaaaactcgtgcagaaatagcaatc |
| | atagaattaaattcttcagttaattatctttataagcttagttgtatttgttttccagaatgtcaaattgttttata |
| | taaaaatcaacattatttgcttccaagtttaataaaaggaataatcgccaataatctagcaatgggtcggtcttggt |
| | gggctaattggatttcatttattaaaaataaaaaatcatttaaacaatttactgatgagtataaaggaattaataag |
| | atgattcaggactcagaatgggatatagaagctcaaaagctatttattcaagcttgtcatgaagcattaagaaaaat |
| | atatgccaaaatttatagcagaacaaaagaaggtgaatacgctcaaatagaacgagaaaatacacgcattttatctc |
| | agttaaaacgctgcaccaatactgacaatttccgaaaatttattgcagagttttggggaagggcagggcaaatagca |
| | attctggaagaacattggatggaattacttccattgactacagggataggcaattggaaagtggccagggacttaac |
| | ttttattgcaatggctagctacccaaagaataaaataacagaggtagaggtaaaaccatcacaaatatcggagctaa |
| | atactgaataaacttcatattttttgatttgattaagaacaaaatattaattgtttaggtaatggtggaatgtttca |
| | tctgtttggtaatattttgactagttatggaaccgccgctaataatcgtggtgaaaatgaaggtaataccactacgt |
| | tacaaaaattaatttggaaaggtgaagttcactctactgtttcatctgaggcaattcgttgggcattgcgatattat |
| | tggcaaaatgctggctatcctgtgaatagacgttgggatgaaaatgctcaaccagtacccgatcacatcttgcaaga |
| | tcctaattttgatgatattcgcttcattgatgatgatgtcttgggatttatgcaagcggaagcagcaaaagttgaag |
| | cagcagttgaatccgaagttgaaatccagaactcaactcaagatgaggacaaaaaactttctcaaggtaaggagaaa |
| | gaaactgcaaaacgtaagcgtaaggaaaaacctaaagggaaaataactactagacgaggtgtattagaagtcactcg |
| | tgcagtttcaacaattccttatgctggtgatcttactttcaatgctatgagtggaaagaaaggacgaacatctcttt |
| | attctacagaagttcatgctactcgatatcaatatggcttcgcattaacacccaatagcctcaaagataaatctcgc |
| | attaatgctgtcctcgatagcttaatttctattggagaagtggcaggaaatcacgctcgttttctctatgatttttc |
| | tcctgatagcatcatattacggtggacacacgatttctcacctcggttactttactgttttgaagaagatgaactta |
| | gaaatatatcaacaccggatttagtaaggcgggtagaatctggagatatagaccctaaagaattatggataggaggg |
| | gcaatttctagcactctggaagatttaggggcaaatgtgtttcccggagtgaagacaacagttgaagcattaaaaca |
| | ggtgattattgaagatttgcagttgtcgttaaggagttcatgatatgacgacaattgccttaaaagtagaagttccc |
| | atagcttgttttcggcagtctcgtgctagggaatatggcgaaacttaccctgtaccaccaccatcgacagtatatgg |
| | aatgttactatctcttgttggagaagtggatagatacaaacactgcggtgtgaagctggcgatcgcacttctatctg |
| | aaccagaaaagtcaactgttattcgcacctttcaccgatttaagacgaagaatatccatgactctaaaaataataaa |
| | cctgattatcaggagttattaactaatatagaatttatagtttgggtggatgctggagtagataaagctaaaccaaa |
| | tttagtggaacgtttagcagaggcattgactaacccggcttctattaataggtttggtggattatgtttaggtgaaa |
| | gtcgggatttagtgaatgatgttacccttttacctaaaaattactatgcagaatcaatgcgatggttaattcgagat |
| | gaatatgggttactaactttaccttactgggtagaccatgtaggttcgcggggaacgcgatggctacgttacgagat |
| | tcagaaatgtccagtctttcaaccacctgaattatcttggacttcagttcaaagtaattagCCTA |
| | GGCTGCTGCCACCGCTGAGCAATAACTAGCATAACCCCTTGGGGCCTCTAAACGGGTCTTGAGGGGTTTTTTGCT |
| | GAAACCTCAGGCATTTGAGAAGCACACGGTCACACTGCTTCCGGTAGTCAATAAACCGGTAAACCAGCAATAGA |
| | CATAAGCGGCTATTTAACGACCCTGCCCTGAACCGACGACCGGGTCATCGTGGCCGGATCTTGCGGCCCCTCGGC |
| | TTGAACGAATTGTTAGACATTATTTGCCGACTACCTTGGTGATCTCGCCTTTCACGTAGTGGACAAATTCTTCCAA |
| | CTGATCTGCGCGCGAGGCCAAGCGATCTTCTTCTTGTCCAAGATAAGCCTGTCTAGCTTCAAGTATGACGGGCTG |
| | ATACTGGGCCGGCAGGCGCTCCATTGCCCAGTCGGCAGCGACATCCTTCGGCGCGATTTTGCCGGTTACTGCGCT |
| | GTACCAAATGCGGGACAACGTAAGCACTACATTTCGCTCATCGCCAGCCCAGTCGGGCGGCGAGTTCCATAGCGT |
| | TAAGGTTTCATTTAGCGCCTCAAATAGATCCTGTTCAGGAACCGGATCAAAGAGTTCCTCCGCCGCTGGACCTAC |
| | CAAGGCAACGCTATGTTCTCTTGCTTTTGTCAGCAAGATAGCCAGATCAATGTCGATCGTGGCTGGCTCGAAGAT |
| | ACCTGCAAGAATGTCATTGCGCTGCCATTCTCCAAATTGCAGTTCGCGCTTAGCTGGATAACGCCACGGAATGAT |
| | GTCGTCGTGCACAACAATGGTGACTTCTACAGCGCGGAGAATCTCGCTCTCTCCAGGGGAAGCCGAAGTTTCCAA |
| | AAGGTCGTTGATCAAAGCTCGCCGCGTTGTTTCATCAAGCCTTACGGTCACCGTAACCAGCAAATCAATATCACT |
| | GTGTGGCTTCAGGCCGCCATCCACTGCGGAGCCGTACAAATGTACGGCCAGCAACGTCGGTTCGAGATGGCGCTC |
| | GATGACGCCAACTACCTCTGATAGTTGAGTCGATACTTCGGCGATCACCGCTTCCCTCATACTCTTCCTTTTTCAA |
| | TATTATTGAAGCATTTATCAGGGTTATTGTCTCATGAGCGGATACATATTTGAATGTATTTAGAAAAATAAACAAA |
| | TAGCTAGCTCACTCGGTCGCTACGCTCCGGGCGTGAGACTGCGGCGGGCGCTGCGGACACATACAAAGTTACCCA |
| | CAGATTCCGTGGATAAGCAGGGGACTAACATGTGAGGCAAAACAGCAGGGCCGCGCCGGTGGCGTTTTTCCATA |
| | GGCTCCGCCCTCCTGCCAGAGTTCACATAAACAGACGCTTTTCCGGTGCATCTGTGGGAGCCGTGAGGCTCAACC |
| | ATGAATCTGACAGTACGGGCGAAACCCGACAGGACTTAAAGATCCCCACCGTTTCCGGCGGGTCGCTCCCTCTTG |
| | CGCTCTCCTGTTCCGACCCTGCCGTTTACCGGATACCTGTTCCGCCTTTCTCCCTTACGGGAAGTGTGGCGCTTTCT |
| | CATAGCTCACACACTGGTATCTCGGCTCGGTGTAGGTCGTTCGCTCCAAGCTGGGCTGTAAGCAAGAACTCCCCG |
| | TTCAGCCCGACTGCTGCGCCTTATCCGGTAACTGTTCACTTGAGTCCAACCCGGAAAAGCACGGTAAAACGCCAC |
| | TGGCAGCAGCCATTGGTAACTGGGAGTTCGCAGAGGATTTGTTTAGCTAAACACGCGGTTGCTCTTGAAGTGTGC |
| | GCCAAAGTCCGGCTACACTGGAAGGACAGATTTGGTTGCTGTGCTCTGCGAAAGCCAGTTACCACGGTTAAGCAG |
| | TTCCCCAACTGACTTAACCTTCGATCAAACCACCTCCCCAGGTGGTTTTTTCGTTTACAGGGCAAAAGATTACGCG |
| | CAGAAAAAAAGGATCTCAAGAAGATCCTTTGATCTTTTCTACTGAACCGCTCTAGATTTCAGTGCAATTTATCTCT |
| | TCAAATGTAGCACCTGAAGTCAGCCCCATACGATATAAGTTGTAATTCTCATGTTAGTCATGCCCCGCGCCCACC |
| | GGAAGGAGCTGACTGGGTTGAAGGCTCTCAAGGGCATCGGTCGAGATCCCGGTGCCTAATGAGTGAGCTAACTT |
| | ACATTAATTGCGTTGCGCTCACTGCCCGCTTTCCAGTCGGGAAACCTGTCGTGCCAGCTGCATTAATGAATCGGCC |
| | AACGCGCGGGGAGAGGCGGTTTGCGTATTGGGCGCCAGGGTGGTTTTTCTTTTCACCAGTGAGACGGGCAACAGC |
| | TGATTGCCCTTCACCGCCTGGCCCTGAGAGAGTTGCAGCAAGCGGTCCACGCTGGTTTGCCCCAGCAGGCGAAAA |
| | TCCTGTTTGATGGTGGTTAACGGCGGGATATAACATGAGCTGTCTTCGGTATCGTCGTATCCCACTACCGAGATGT |
| | CCGCACCAACGCGCAGCCCGGACTCGGTAATGGCGCGCATTGCGCCCAGCGCCATCTGATCGTTGGCAACCAGCA |
| | TCGCAGTGGGAACGATGCCCTCATTCAGCATTTGCATGGTTTGTTGAAAACCGGACATGGCACTCCAGTCGCCTT |
| | CCCGTTCCGCTATCGGCTGAATTTGATTGCGAGTGAGATATTTATGCCAGCCAGCCAGACGCAGACGCGCCGAGA |
| | CAGAACTTAATGGGCCCGCTAACAGCGCGATTTGCTGGTGACCCAATGCGACCAGATGCTCCACGCCCAGTCGCG |
| | TACCGTCTTCATGGGAGAAAATAATACTGTTGATGGGTGTCTGGTCAGAGACATCAAGAAATAACGCCGGAACAT |
| | TAGTGCAGGCAGCTTCCACAGCAATGGCATCCTGGTCATCCAGCGGATAGTTAATGATCAGCCCACTGACGCGTT |
| | GCGCGAGAAGATTGTGCACCGCCGCTTTACAGGCTTCGACGCCGCTTCGTTCTACCATCGACACCACCACGCTGG |
| | CACCCAGTTGATCGGCGCGAGATTTAATCGCCGCGACAATTTGCGACGGCGCGTGCAGGGCCAGACTGGAGGTG |
| | GCAACGCCAATCAGCAACGACTGTTTGCCCGCCAGTTGTTGTGCCACGCGGTTGGGAATGTAATTCAGCTCCGCC |
| | ATCGCCGCTTCCACTTTTTCCCGCGTTTTCGCAGAAACGTGGCTGGCCTGGTTCACCACGCGGGAAACGGTCTGAT |
| | AAGAGACACCGGCATACTCTGCGACATCGTATAACGTTACTGGTTTCACATTCACCACCCTGAATTGACTCTCTTC |
| | CGGGCGCTATCATGCCATACCGCGAAAGGTTTTGCGCCATTCGATGGTGTCCGGGATCTCGACGCTCTCCCTTATG |
| | CGACTCCTGCATTAGGAAATTAATACGACTCACTATA (SEQ ID NO: 44) |
| pDonor_CP000117 | AGCTTGCCATTGCAACTATTGGCTTACCACATCGCCGCCCGTCGCGGTTTGGATGTCGATCAGCCTCGGAATTTAG |
| | CAAAATCAGTGACTGTGGAATAAATAAATGTGGAGGAATAATTCGGTTCAACAATATATAATTCGGTTCAACAAT |
| | TAATAAAACGATTGAACAATAACCTCTAAAGGAGTACGATTGTACCAAGTAAAAGTGCGATCGCTTCCAGAGGTT |
| | ATTTTTATGGTTAGGGGTAGAAGGGAATGGCGGGGATCCATGGAGAAAAAAATCACTGGATATACCACCGTTGA |
| | TATATCCCAATGGCATCGTAAAGAACATTTTGAGGCATTTCAGTCAGTTGCTCAATGTACCTATAACCAGACCGTT |
| | CAGCTGGATATTACGGCCTTTTTAAAGACCGTAAAGAAAAATAAGCACAAGTTTTATCCGGCCTTTATTCACATTC |
| | TTGCCCGCCTGATGAATGCTCATCCGGAGTTCCGTATGGCAATGAAAGACGGTGAGCTGGTGATATGGGATAGTG |
| | TTCACCCTTGTTACACCGTTTTCCATGAGCAAACTGAAACGTTTTCATCGCTCTGGAGTGAATACCACGACGATTT |
| | CCGGCAGTTTCTACACATATATTCGCAAGATGTGGCGTGTTACGGTGAAAACCTGGCCTATTTCCCTAAAGGGTTT |
| | ATTGAGAATATGTTTTTCGTCTCAGCCAATCCCTGGGTGAGTTTCACCAGTTTTGATTTAAACGTGGCCAATATGG |
| | ACAACTTCTTCGCCCCCGTTTTCACTATGGGCAAATATTATACGCAAGGCGACAAGGTGCTGATGCCGCTGGCGA |
| | TTCAGGTTCATCATGCCGTTTGTGATGGCTTCCATGTCGGCAGAATGCTTAATGAATTACAACAGTACTGCGATGA |
| | GTGGCAGGGCGGGGCGTAATTTTTTTAAGGCAGTTATTGGTGCCCTTCTAGAGTCCCGGGATATTTACGTTTTGTA |
| | ATCACGAATAGATTCTTGATTGCAAGATGAATTGCGATCGCATCTTTGTCGCACAGCAGAACTTTCTCAAATAATT |
| | TTGAACTTATGGAATTACTCAAAACCTTTATCAGTAATGGATTCAAACAATTGTTTAACCGTTTTATTTGAGATTTT |
| | TACTAAAAATCAAGTTTACCATAGAGGTTAAGCTGAAATTGTTCAATCGTTTTATTTTATCGACAACGGCAGATTA |
| | AATATTCGAACTGTAATAATTGTTTAACCGAATTCTTTGTCTACAAATATTTGTAGAGTAGATAAGATAATTTCTA |
| | GATGTGACTACTATATCACATCTAGGTACCGAGCTCGAATTCACTGGCCGTCGTTTTACAACGTCGTGACTGGGA |
| | AAACCCTGGCGTTACCCAACTTAATCGCCTTGCAGCACATCCCCCTTTCGCCAGCTGGCGTAATAGCGAAGAGGC |
| | CCGCACCGATCGCCCTTCCCAACAGTTGCGCAGCCTGAATGGCGAATGGCGCCTGATGCGGTATTTTCTCCTTACG |
| | CATCTGTGCGGTATTTCACACCGCATATGGTGCACTCTCAGTACAATCTGCTCTGATGCCGCATAGTTAAGCCAGC |
| | CCCGACACCCGCCAACACCCGCTGACGCGCCCTGACGGGCTTGTCTGCTCCCGGCATCCGCTTACAGACAAGCTG |
| | TGACCGTCTCCGGGAGCTGCATGTGTCAGAGGTTTTCACCGTCATCACCGAAACGCGCGAGACGAAAGGGCCTCG |
| | TGATACGCCTATTTTTATAGGTTAATGTCATGATAATAATGGTTTCTTAGACGTCAGGTGGCACTTTTCGGGGAAA |
| | TGTGCGCGGAACCCCTATTTGTTTATTTTTCTAAATACATTCAAATATGTATCCGCTCATGAGACAATAACCCTGA |
| | TAAATGCTTCAATAATATTGAAAAAGGAAGAGTATGAGTATTCAACATTTCCGTGTCGCCCTTATTCCCTTTTTTG |
| | CGGCATTTTGCCTTCCTGTTTTTGCTCACCCAGAAACGCTGGTGAAAGTAAAAGATGCTGAAGATCAGTTGGGTG |
| | CACGAGTGGGTTACATCGAACTGGATCTCAACAGCGGTAAGATCCTTGAGAGTTTTCGCCCCGAAGAACGTTTTC |
| | CAATGATGAGCACTTTTAAAGTTCTGCTATGTGGCGCGGTATTATCCCGTATTGACGCCGGGCAAGAGCAACTCG |
| | GTCGCCGCATACACTATTCTCAGAATGACTTGGTTGAGTACTCACCAGTCACAGAAAAGCATCTTACGGATGGCA |
| | TGACAGTAAGAGAATTATGCAGTGCTGCCATAACCATGAGTGATAACACTGCGGCCAACTTACTTCTGACAACGA |
| | TCGGAGGACCGAAGGAGCTAACCGCTTTTTTGCACAACATGGGGGATCATGTAACTCGCCTTGATCGTTGGGAAC |
| | CGGAGCTGAATGAAGCCATACCAAACGACGAGCGTGACACCACGATGCCTGTAGCAATGGCAACAACGTTGCGC |
| | AAACTATTAACTGGCGAACTACTTACTCTAGCTTCCCGGCAACAATTAATAGACTGGATGGAGGCGGATAAAGTT |
| | GCAGGACCACTTCTGCGCTCGGCCCTTCCGGCTGGCTGGTTTATTGCTGATAAATCTGGAGCCGGTGAGCGTGGG |
| | TCTCGCGGTATCATTGCAGCACTGGGGCCAGATGGTAAGCCCTCCCGTATCGTAGTTATCTACACGACGGGGAGT |
| | CAGGCAACTATGGATGAACGAAATAGACAGATCGCTGAGATAGGTGCCTCACTGATTAAGCATTGGTAACTGTC |
| | AGACCAAGTTTACTCATATATACTTTAGATTGATTTAAAACTTCATTTTTAATTTAAAAGGATCTAGGTGAAGATC |
| | CTTTTTGATAATCTCATGACCAAAATCCCTTAACGTGAGTTTTCGTTCCACTGAGCGTCAGACCCCGTAGAAAAGA |
| | TCAAAGGATCTTCTTGAGATCCTTTTTTTCTGCGCGTAATCTGCTGCTTGCAAACAAAAAAACCACCGCTACCAGC |
| | GGTGGTTTGTTTGCCGGATCAAGAGCTACCAACTCTTTTTCCGAAGGTAACTGGCTTCAGCAGAGCGCAGATACC |
| | AAATACTGTTCTTCTAGTGTAGCCGTAGTTAGGCCACCACTTCAAGAACTCTGTAGCACCGCCTACATACCTCGCT |
| | CTGCTAATCCTGTTACCAGTGGCTGCTGCCAGTGGCGATAAGTCGTGTCTTACCGGGTTGGACTCAAGACGATAG |
| | TTACCGGATAAGGCGCAGCGGTCGGGCTGAACGGGGGGTTCGTGCACACAGCCCAGCTTGGAGCGAACGACCTA |
| | CACCGAACTGAGATACCTACAGCGTGAGCTATGAGAAAGCGCCACGCTTCCCGAAGGGAGAAAGGCGGACAGGT |
| | ATCCGGTAAGCGGCAGGGTCGGAACAGGAGAGCGCACGAGGGAGCTTCCAGGGGGAAACGCCTGGTATCTTTAT |
| | AGTCCTGTCGGGTTTCGCCACCTCTGACTTGAGCGTCGATTTTTGTGATGCTCGTCAGGGGGGCGGAGCCTATGGA |
| | AAAACGCCAGCAACGCGGCCTTTTTACGGTTCCTGGCCTTTTGCTGGCCTTTTGCTCACATGTTCTTTCCTGCGTTA |
| | TCCCCTGATTCTGTGGATAACCGTATTACCGCCTTTGAGTGAGCTGATACCGCTCGCCGCAGCCGAACGACCGAG |
| | CGCAGCGAGTCAGTGAGCGAGGAAGCGGAAGAGCGCCCAATACGCAAACCGCCTCTCCCCGCGCGTTGGCCGAT |
| | TCATTAATGCAGCTGGCACGACAGGTTTCCCGACTGGAAAGCGGGCAGTGAGCGCAACGCAATTAATGTGAGTT |
| | AGCTCACTCATTAGGCACCCCAGGCTTTACACTTTATGCTTCCGGCTCGTATGTTGTGTGGAATTGTGAGCGGATA |
| | ACAATTTCACACAGGAAACAGCTATGACCATGATTACGCCA (SEQ ID NO: 45) |
| pTns_CP000117 | ggggaattgtgagcggataacaattcccctgtagaaataattttgtttaactttaataaggagatataccatggt |
| | taggggtagaagggaatggactcaagctaaatatgagcgatatataaaagaaggtcgtggccgaggaagtggtaa |
| | gcactataaaccttggcttacaatccaagatgttccctctaaaggacgttcttctagaactcctggttggaaaac |
| | aaatcgagtacatcatttcttctcagaccatgaaaaacggctattttatttgtttgagtggtcagatatagtcac |
| | agatataagagagcaatttcctttagacaatctcgatctagccatgagtattgctacagacataggtataaagta |
| | ccctgtagatacacaaagtggcactcctcacgttttaacaagcgactttatgcttacagtcaaccgcgatgggaa |
| | gcagatgcagattgcccggactgttaagccgtcaacagaactggagaaaaagcaagttaatgagaaattcgagat |
| | agaaagacaatattacttagcaaagggtattgactggggaattatcacagaaaaagaaattcccaggcttttagc |
| | tgaaaatgttgagtgggttcattctgcttataggctagaagcaaattctgaaatggacactgcacaactgagtga |
| | attgtcaacaattttaaaatctaagcttcaggaagaagatacaactataaatagagtaactactgctttagacaa |
| | agaaatgaatataaattccggtacatcactttatgtatttaaacatttaattgctaggaaagaaatccttgtgga |
| | tctagtagcaataaaattatctgcttgtccgtcaactaaagttattgaaaaaataatattttagacccacatgat |
| | aggtaaataatatggttaaagatttatttgtcaatgatttaatagagtggattgtataccaaaacactcatatag |
| | tcgaaagaattatttggatagatgaaggttacattattgcatttgttatagatattaatgctaagaaaggtctac |
| | ctgaaccaaggaaaatatcagatattttagaagctataactgaaggtattgcttttaagcagcaacaagatcctt |
| | gggcaagaattattcgggatgaaaatttgacagataaggaaaaggaatacagagataaagcttgggagataattt |
| | catcattaattatagaagaaccatcaatatattatagaaattttagaggatcgatggttaaagaagcggttgaaa |
| | agtacaacgaagaaagggataaagataaattagtagaaaaaaccgtctatggttatttgagaagattttggcaga |
| | gaggaaaaactataaatgctctcatacctgattttatcaattcatcaggtaagggcaagattcgaggacaatcgg |
| | gcaagaagagaggaagaccaagaaaatatgcacatgacccagatattggtgaaggtgtaaatgttacagaagaag |
| | atagaaagatttttagagtatctataagcaaattttacaataatccaaaagaaaactacttaacaacagccta |
| | tgaattaatggtgaaagaatattacaaggaaggaattagatatgacgagaatggtgttcaaaaatctatattaac |
| | cccaactgataaaatacctacaattactcaattcaaatactggtatcaacttgaacaggatgatgttaccaaaac |
| | tttaacatctcgaaaaggagccaagcgagtagctttagagaatagagcaattttgggaaactctaagatggaaac |
| | tattggccctggttctagatatcaaattgatgccacagttgctgatgtatatttagtttcaaaatataaccgtga |
| | ctggataattggtagaccagttatctatgtaattattgatgtttttagtagaatgattacaggtgtttatgtcgg |
| | tttagaaggcccatcttggataggagcaatgatggcactagccaatgtagctacagataaaaccctgttttgcca |
| | ggaatataacattaatattactgaaaatgaatggccttgcaatcaaatgccagatgcaattttaggagatagggg |
| | agaattagcaggtatgacagtagaaacattaatccctaatctttatgttcgtattgaaaatgcagcttcctatag |
| | agcagattggaaagggttagtagaaagacactttcgtataattcatgaatatgtaaaaccttttttacctggtta |
| | tgtggatacggattttaggcaaagaggtgcaagggattataggcttgatggcaaacttgatatagatcaatttac |
| | tgagattattattcgtttaattgttcaccataacaataaacctcttactgaatatgaaagagatgaagcaatgat |
| | tgctgatgatacccctcctatacctagagagttgtggaagtggggtatcgccaatcgttctggcagactcagaac |
| | tttttctgaagatattgtcaaattgaacttgatgcctactgagaaagcaactattactgctaaaggcatcaaaat |
| | gagaggtaaggagatgtactatacctgtgatagtgctaaaaatgagcaatggtttgaaaaagctagaagtaatct |
| | cctttctaagtcagaaaaatcattgattgtatcttatgacatcagaaagccaaacttcatttatcttccatctag |
| | tgatggtagagattttgaaaaatgttttcttattgacttagaagggcggtatgctaataaaaactttcatgatat |
| | tgaatatctgcttgcttatgaagaattgcaaagacagaaaagccaaggtaaacagctacaagaaaaggtagattt |
| | aatagctgatattgaaagcatagtagatagagcagaaaaaatgactgatgcttctcaatatcataatttaagtaa |
| | gagcaagaaagtttctggaataagagataatagggcttttgaaaaggctaaacgacgagagaatgaaggatttga |
| | attagcaattacagataatcagagcagttttcaggttgcagagagtacagcagaaacctccgaacagccagaatc |
| | actacagccaaaccatctgaatcttcttaagaaaaagcgacaggagcgaaaacgtggacagaattgagagtgaac |
| | tggttaaaattcctaatggagaactagcagttattgccagatatatagatcaaaaactgccagaatacaacacta |
| | atccattaattcaggcattacctcctatcctatcagcagcagaatttatagataaagtagccagagaaccaaatt |
| | ttgatgaacaagagagagagctagaaccacattatagatttcattgtattgagaggttatctagatattttgacc |
| | ctcaaaataaaactcttgaattacaaaaagttatttgtacgcttatcatgacaggctatctgtcaaggaaccttt |
| | taaaacctgaatatgcaaatcgctctaggcaaatttataatgcaataggtaatggaggaggtaaaaatttagaaa |
| | actatgttaatcttcctacatctgcgtccggcttaactctaattggcccatcaggtattggaaagactacaaatt |
| | tcaaaaatattcttgagctttatccccaaataatttttcatcctgattatagtgtttaccaaatagtttggctgc |
| | gggtagattgccctaaaactttaaaggggttatgtatagatacttttgcaggtatcgataaattgttaggaacta |
| | atttattaaagaaatttaacccagcacgcacatctgaagattatctattggctcaacttgcacaaacagcacaca |
| | ctcatcatctaggagttttggtgattgatgaaatacaaaatttagcaactgcaaaaagaaatagagacgaaatat |
| | taaattttttagtaaagctcgataatactattggaattccagttattcgtgtaggtacgaatgaagccattagtt |
| | ttcttcaaggtaatcttagaaatgctagaagaaatactggagaaggaagtagaatgtgggatagaatggtaaata |
| | atgatgaatggtgttactttttagaaggaatatgggaataccaatggacaaaaacacctgttcatttctcggatg |
| | aaataaaagaagttttttatgatgaaagccagggaattattgatattgctataaaactttacaaaatggttcaat |
| | ggagagcgatttcaataggtgaccaagaaattattacggttgatctgattcgtcaagtagctaaagaaggattgt |
| | atttagtaaagccaatgctagatgcaattaggttaggggacagagagtggatgattaaatataaggatattgctc |
| | ctctcgatattacggaatatcacaatagatgtgtatctgagattgatgctaaagatttgacagagattaagagat |
| | tagctaaaaagcagcagaatcagcagaaagtatctccgaagctcaattatgtaattagtgagctgttgaaattgg |
| | aagtagaatctggtaaagcaaaggaatgtgcagaacaaattgtttatagtcatgaagatccagacattccaattc |
| | catctttagttaataaggcatatgctcttgctttgcaaggatgtcaagtaaatgagagtcttgctaaaacaagta |
| | ccaggaaagctaaattaaagacaaagtaccaagagaatgatattcggcgaattgtcgagaatgccaagaaagaca |
| | aaaaatctgcatacgaggatttgaaattagcaggtataatcaaaaatccagttgaagattttatcaatatttagc |
| | ttctaaatgcttagttttttcccaactctgtatcccgatgaacttttatatagcactttagctagataccatatt |
| | cggagtggaaataaaagtttcaagcaaactgacttagagctattcggttacagttcccaacaaatatgcaaaatt |
| | accttaactaataatttaaattatttagtgaaaaacttacctttacgctcccagcaaaaagttgagaatttacta |
| | caaaatcatactttatattccttttatgcaactttattaatgccgcaggaggcttggctgctgaaaaactcaatg |
| | agtaataaattcagtgggtctatactagattttgctaaagtggctaataattctagtgatgattcaaaaaaattt |
| | ttaaaattttgccctgaatgtttagagaaagatacccaaaagtatggtgaaccttactggcatagaatacatcaa |
| | gtccctggcattttagtttgtccaattcatcgtgttgttttacacaatagctgtatagaagttgaatcgaaaggc |
| | atacattatcatgctgccagcatagataattgtttattagcaaataatgtgaaaaaattgacaaataaaactgta |
| | gaaacactttcaatactggcacatgatattaattggctaattcatagcagttttgcctttcaaggtttggcttgg |
| | cttcgcgctcagtaccagcgttacttattggataaaggactgctaaaaacctttgctggtggcaagtttaagttt |
| | aatgattgtggttttgcccagcttatatttgagttttatggtcaagaatccttagaagccctcaatccaaagctg |
| | acccaaaatcaaggtaaatatttttctcactgcttgtttggatgcgacttaaacccagcaattgaccgcataact |
| | catatcttaatgataaaatttttagcaaattctatagaagagttctttcagtcataacttaagtcgaacagaaag |
| | taatcgtattgtacacggccgcataatcgaaattaatacgactcactataggggaattgtgagcggataacaatt |
| | ccccatcttagtatattagttaagtataagaaggagatatacatggtgagttttttccctcaaccttatccagat |
| | gaaattttatatagtgttattgctcgttatcacattagaagtggcaatacgagtccaaaaattacattacaagat |
| | ttatttaagtcacagactactgtggcgacagtagatttaccatcgaaccttagtaatttaattaaaaacctacaa |
| | tttatctcaaattaccaagttgaagagttgatatataaacatactctctaccctttatatagcccttttttgcca |
| | gcaaacaggtctagtcaggtactggaatctatgaaaggagattatgggggagatattcatacaagagccggaatt |
| | atggcaagctcaattactatgccgaaatatttgcggttttgtccaacttgctgggaagaagacttaaaaaattat |
| | ggcgaaccttactggcatcgcatccatcaaataccagggattttagtttgtccatttcatggtgaagttttgcaa |
| | gatagcctagctcctttacatggttttaataagcatgaatattatgcagctagtacagacaactgttctattact |
| | aaagaacccagtattcttagccctgacacactgaaaaaattactaatattagccaaagatttttggtggttactc |
| | aatagtgaaataccttgccaagagccacagtggtttcgcaaacagtacacaaatctgatgattgagcagggtttt |
| | gcaactccaactggtcgaatttaccagaacaaactactagataattttttgttcttttatggtcaagaaatgctc |
| | agtgctgttgattcaatggtaaatcataatgattcaaacaactgggttactagcatcgtcaggaaatataggaaa |
| | tctttccatcctcttagacatctgctaatgatgcgttttttgaaaacatcagtagaggaattttttcaaaaagat |
| | actgaatacaagccttttggcgaacccccctggttgtgtctcaatgctgccgcagaacattatctacaaccag |
| | tgattaccaatttagtcattactttatgctgcgatacaaaaaagcctgtcggtactttttcttgtgcttgcggaa |
| | tggtttattgccggactgggcctgatgaaacggatgaagacaaagttcgcattggtaaagtcaaggcattcggtc |
| | aaatatgggagcaaaagctgaaagaattggtggaagttgaaaagttgggtttgcgggagacagccagaagattaa |
| | atgttgatgccagaactatcaaacgttatgtatctcgtctaaaattgacaacttattggcagactagaacagaaa |
| | atgactctgtaggcttacagggagtcccagaaatagatcccaattttgcaactgaactcaaattaaaacacag |
| | gcaaaattggatggctttacaagtacagaaccctgaagcttcaaagacaactctgagaaaactagtaccagccac |
| | ctatatttggctatacaggaatgacagagaatggctcaatcaaaactcacccactttacaagttgccgttccttc |
| | cgttgtgagagttgattggcatgagcgagatcagcaaattctcgctcaggttcaagatgcagtgcgatcgctact |
| | taaggctgatataccagagaaaatttctattagtagggtagggaaaactatcggcttacttgcattactcgaaaa |
| | acatctcgaccagatgcctttaacaaaggcttatttggaatcagtcgttgaaactgtagaagaatttcagatgcg |
| | ccggatcaagtgggcaatcaagctgcttgatgaatgtggagaagaaattatgcgctggaaggtagtcagagttgc |
| | tgctttgcgagaggattgctcccaaagagtcagtgcatttctggaaagtgaactggagaaagcatacaagaaaga |
| | gaggtgattaattaacctaggctgctgccaccgctgagcaataactagcataaccccttggggcctctaaacggg |
| | tcttgaggggttttttgctgaaacctcaggcatttgagaagcacacggtcacactgcttccggtagtcaataaac |
| | cggtaaaccagcaatagacataagcggctatttaacgaccctgccctgaaccgacgacaagctgacgaccgggtc |
| | tccgcaagtggcacttttcggggaaatgtgcgcggaacccctatttgtttatttttctaaatacattcaaatatg |
| | tatccgctcatgaattaattcttagaaaaactcatcgagcatcaaatgaaactgcaatttattcatatcaggatt |
| | atcaataccatatttttgaaaaagccgtttctgtaatgaaggagaaaactcaccgaggcagttccataggatggc |
| | aagatcctggtatcggtctgcgattccgactcgtccaacatcaatacaacctattaatttcccctcgtcaaaaat |
| | aaggttatcaagtgagaaatcaccatgagtgacgactgaatccggtgagaatggcaaaagtttatgcatttcttt |
| | ccagacttgttcaacaggccagccattacgctcgtcatcaaaatcactcgcatcaaccaaaccgttattcattcg |
| | tgattgcgcctgagcgagacgaaatacgcggtcgctgttaaaaggacaattacaaacaggaatcgaatgcaaccg |
| | gcgcaggaacactgccagcgcatcaacaatattttcacctgaatcaggatattcttctaatacctggaatgctgt |
| | tttcccggggatcgcagtggtgagtaaccatgcatcatcaggagtacggataaaatgcttgatggtcggaagagg |
| | cataaattccgtcagccagtttagtctgaccatctcatctgtaacatcattggcaacgctacctttgccatgttt |
| | cagaaacaactctggcgcatcgggcttcccatacaatcgatagattgtcgcacctgattgcccgacattatcgcg |
| | agcccatttatacccatataaatcagcatccatgttggaatttaatcgcggcctagagcaagacgtttcccgttg |
| | aatatggctcatactcttcctttttcaatattattgaagcatttatcagggttattgtctcatgagcggatacat |
| | atttgaatgtatttagaaaaataaacaaataggcatgctagcgcagaaacgtcctagaagatgccaggaggatac |
| | ttagcagagagacaataaggccggagcgaagccgtttttccataggctccgcccccctgacgaacatcacgaaat |
| | ctgacgctcaaatcagtggtggcgaaacccgacaggactataaagataccaggcgtttccccctgatggctccct |
| | cttgcgctctcctgttcccgtcctgcggcgtccgtgttgtggtggaggctttacccaaatcaccacgtcccgttc |
| | cgtgtagacagttcgctccaagctgggctgtgtgcaagaaccccccgttcagcccgactgctgcgccttatccgg |
| | taactatcatcttgagtccaacccggaaagacacgacaaaacgccactggcagcagccattggtaactgagaatt |
| | agtggatttagatatcgagagtcttgaagtggtggcctaacagaggctacactgaaaggacagtatttggtatct |
| | gcgctccactaaagccagttaccaggttaagcagttccccaactgacttaaccttcgatcaaaccgcctccccag |
| | gcggttttttcgtttacagagcaggagattacgacgatcgtaaaaggatctcaagaagatcctttacggattccc |
| | gacaccatcactctagatttcagtgcaatttatctcttcaaatgtagcacctgaagtcagccccatacgatataa |
| | gttgtaattctcatgttagtcatgccccgcgcccaccggaaggagctgactgggttgaaggctctcaagggcatc |
| | ggtcgagatcccggtgcctaatgagtgagctaacttacattaattgcgttgcgctcactgcccgctttccagtcg |
| | ggaaacctgtcgtgccagctgcattaatgaatcggccaacgcgcggggagaggcggtttgcgtattgggcgccag |
| | ggtggtttttcttttcaccagtgagacgggcaacagctgattgcccttcaccgcctggccctgagagagttgcag |
| | caagcggtccacgctggtttgccccagcaggcgaaaatcctgtttgatggtggttaacggcgggatataacatga |
| | gctgtcttcggtatcgtcgtatcccactaccgagatgtccgcaccaacgcgcagcccggactcggtaatggcgcg |
| | cattgcgcccagcgccatctgatcgttggcaaccagcatcgcagtgggaacgatgccctcattcagcatttgcat |
| | ggtttgttgaaaaccggacatggcactccagtcgccttcccgttccgctatcggctgaatttgattgcgagtgag |
| | atatttatgccagccagccagacgcagacgcgccgagacagaacttaatgggcccgctaacagcgcgatttgctg |
| | gtgacccaatgcgaccagatgctccacgcccagtcgcgtaccgtcttcatgggagaaaataatactgttgatggg |
| | tgtctggtcagagacatcaagaaataacgccggaacattagtgcaggcagcttccacagcaatggcatcctggtc |
| | atccagcggatagttaatgatcagcccactgacgcgttgcgcgagaagattgtgcaccgccgctttacaggcttc |
| | gacgccgcttcgttctaccatcgacaccaccacgctggcacccagttgatcggcgcgagatttaatcgccgcgac |
| | aatttgcgacggcgcgtgcagggccagactggaggtggcaacgccaatcagcaacgactgtttgcccgccagttg |
| | ttgtgccacgcggttgggaatgtaattcagctccgccatcgccgcttccactttttcccgcgttttcgcagaaac |
| | gtggctggcctggttcaccacgcgggaaacggtctgataagagacaccggcatactctgcgacatcgtataacgt |
| | tactggtttcacattcaccaccctgaattgactctcttccgggcgctatcatgccataccgcgaaaggttttgcg |
| | ccattcgatggtgtccgggatctcgacgctctcccttatgcgactcctgcattaggaaattaatacgactcacta |
| | ta (SEQ ID NO: 46) |

The sequences of the left end (LE) sequence element and right end (RE) sequence element on the pDonor CP000117 are shown in the Table 6 below.

TABLE 7

| Elements | Sequences |
|---|---|
| LE | CCGGGATATTTACGTTTTGTAATCACGAATAGATTCTTGATTGCAAGATGAATTGCGATCGCATCTTT |
| (pDonor_CP000117 | GTCGCACAGCAGAACTTTCTCAAATAATTTTGAACTTATGGAATTACTCAAAACCTTTATCAGTAATG |
| (962..1261) (300 bp)) | GATTCAAACAATTGTTTAACCGTTTTATTTGAGATTTTTACTAAAAATCAAGTTTACCATAGAGGTTAA |
| | GCTGAAATTGTTCAATCGTTTTATTTTATCGACAACGGCAGATTAAATATTCGAACTGTAATAATTGTT |
| | TAACCGAATTCTTTGTCTACAAATAT (SEQ ID NO: 47) |
| RE | TGTGGAGGAATAATTCGGTTCAACAATATATAATTCGGTTCAACAATTAATAAAACGATTGAACAAT |
| (pDonor_CP000117 | AACCTCTAAAGGAGTACGATTGTACCAAGTAAAAGTGCGATCGCTTCCAGAGGTTATTTTTATGGTTA |
| (105..256) (152 bp)) | GGGGTAGAAGGGAATGG (SEQ ID NO: 48) |

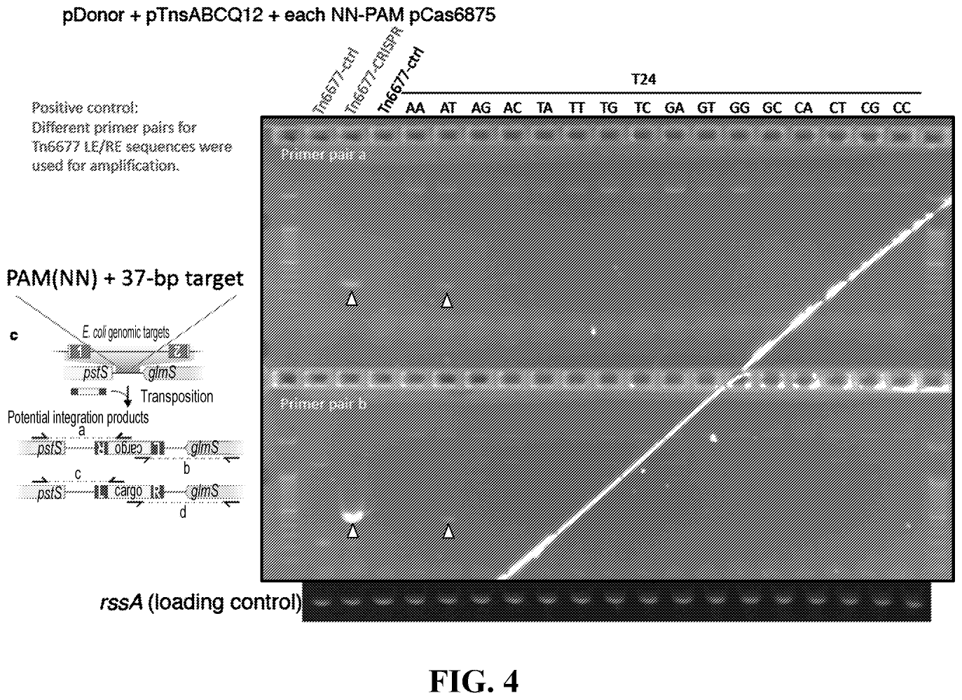

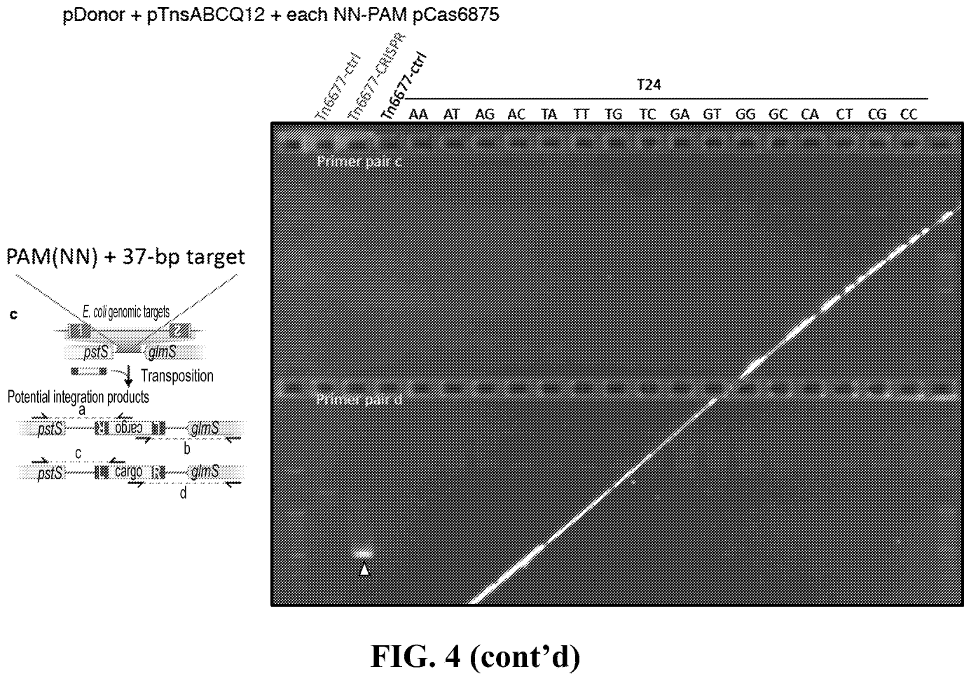

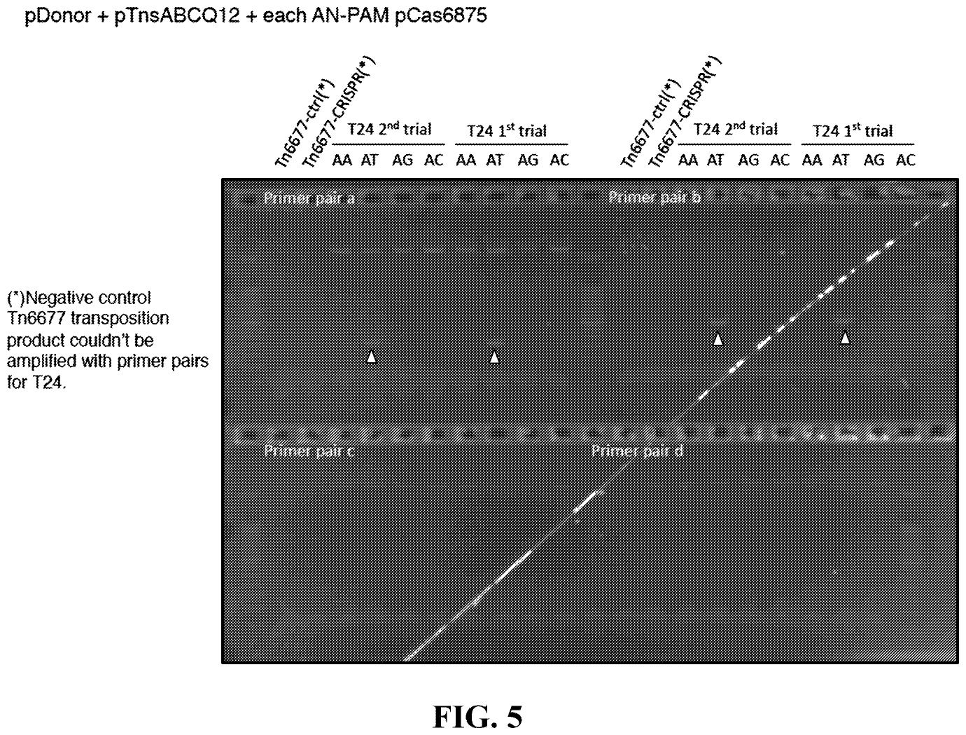

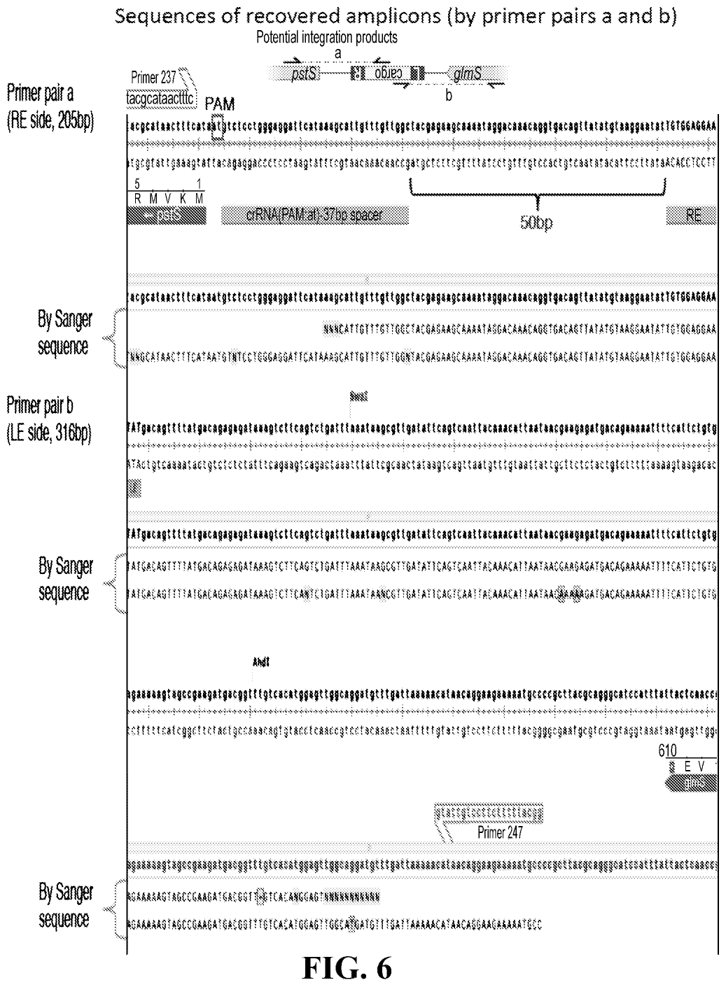

The pCas6875 plasmids that carried the Cas proteins and different NN-PAM were used to test the effects of different PAM sequences. Different primer pairs for Tn6677 LE/RE sequences were used for amplification as positive controls. FIG. 4 shows the test result with primer pairs a, b, c, and d in one set of experiment. FIG. 5 shows the test result with primer pairs a and c in another set of experiment. The gel bands of the amplicons (by primer pairs a and b) were recovered and determined (FIG. 6). In the experiments, a variety of 37-bp sequences were used as intended insertion sequences, which are shown in Table 7 below.

TABLE 8

| ID | Description | Encoded by | PAM | 37bp spacer sequence (5′→3′) |
|---|---|---|---|---|
| crRNA-1 | Targeting genome | pCascade_CP000117 | aa | tgtctcctgggaggattcataaagcattgtttgttgg |
| | (glmS locus) | | | (SEQ ID NO: 49) |
| crRNA-2 | Targeting genome | pCascade_CP000117 | at | gtctcctgggaggattcataaagcattgtttgttggc |
| | (glmS locus) | | | (SEQ ID NO: 50) |
| crRNA-3 | Targeting genome | pCascade_CP000117 | ag | gattcataaagcattgtttgttggctacgagaagcaa |
| | (glmS locus) | | | (SEQ ID NO: 51) |
| crRNA-4 | Targeting genome | pCascade_CP000117 | ac | gagaagcaaaataggacaaacaggtgacagttatatg |
| | (glmS locus) | | | (SEQ ID NO: 52) |
| crRNA-5 | Targeting genome | pCascade_CP000117 | ta | aagcattgtttgttggctacgagaagcaaaataggac |
| | (glmS locus) | | | (SEQ ID NO: 53) |
| crRNA-6 | Targeting genome | pCascade_CP000117 | tt | cataaagcattgtttgttggctacgagaagcaaaata |
| | (glmS locus) | | | (SEQ ID NO: 54) |
| crRNA-7 | Targeting genome | pCascade_CP000117 | tg | tctcctgggaggattcataaagcattgtttgttggct |
| | (glmS locus) | | | (SEQ ID NO: 55) |
| crRNA-8 | Targeting genome | pCascade_CP000117 | tc | tcctgggaggattcataaagcattgtttgttggctac |
| | (glmS locus) | | | (SEQ ID NO: 56) |
| crRNA-9 | Targeting genome | pCascade_CP000117 | ga | ggattcataaagcattgtttgttggctacgagaagca |
| | (glmS locus) | | | (SEQ ID NO: 57) |
| crRNA-10 | Targeting genome | pCascade_CP000117 | gt | ctcctgggaggattcataaagcattgtttgttggcta |
| | (glmS locus) | | | (SEQ ID NO: 58) |
| crRNA-11 | Targeting genome | pCascade_CP000117 | gg | gaggattcataaagcattgtttgttggctacgagaag |
| | (glmS locus) | | | (SEQ ID NO: 59) |
| crRNA-12 | Targeting genome | pCascade_CP000117 | gc | attgtttgttggctacgagaagcaaaataggacaaac |
| | (glmS locus) | | | (SEQ ID NO: 60) |
| crRNA-13 | Targeting genome | pCascade_CP000117 | ca | taaagcattgtttgttggctacgagaagcaaaatagg |
| | (glmS locus) | | | (SEQ ID NO: 61) |
| crRNA-14 | Targeting genome | pCascade_CP000117 | ct | cctgggaggattcataaagcattgtttgttggctacg |
| | (glmS locus) | | | (SEQ ID NO: 62) |
| crRNA-15 | Targeting genome | pCascade_CP000117 | eg | agaagcaaaataggacaaacaggtgacagttatatgt |
| | (glmS locus) | | | (SEQ ID NO: 63) |
| crRNA-16 | Targeting genome | pCascade_CP000117 | cc | tgggaggattcataaagcattgtttgttggctacgag |
| | (glmS locus) | | | (SEQ ID NO: 64) |

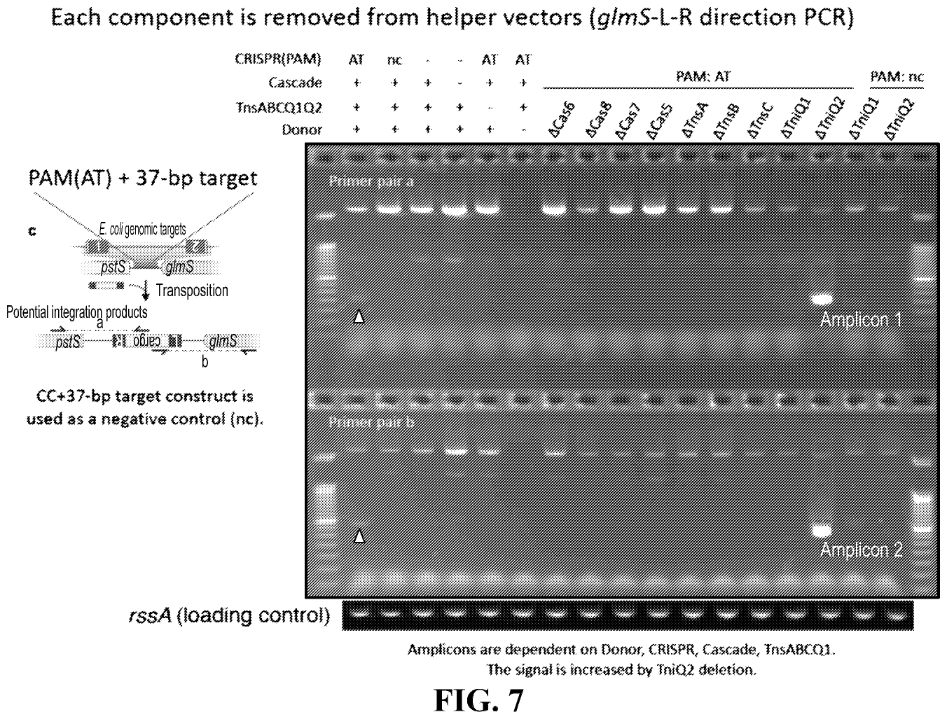

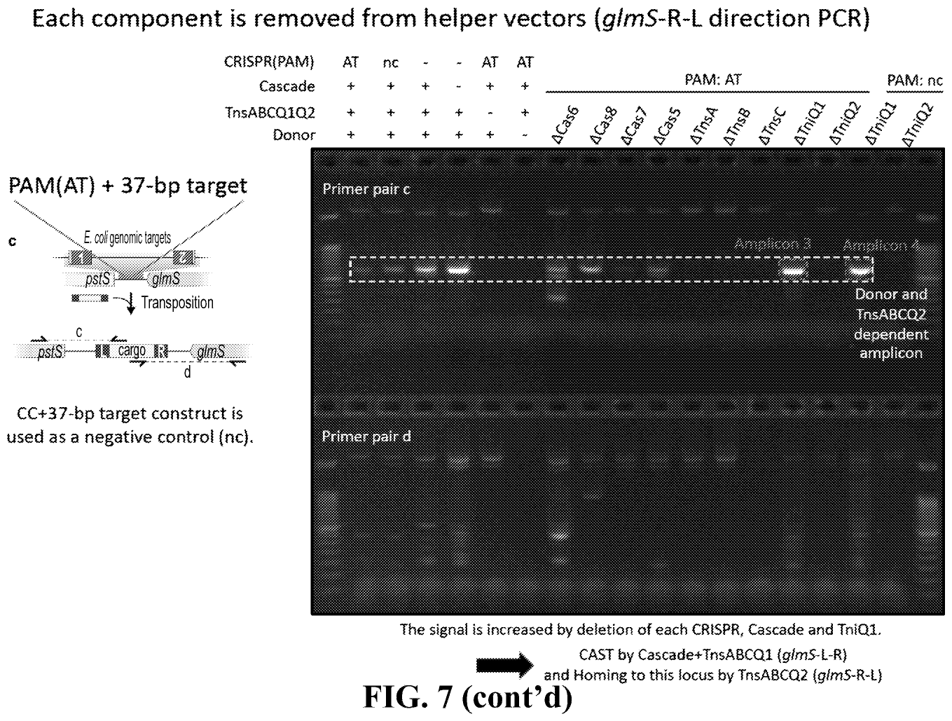

The roles of the components of the CAST system were examined. Each of the components was deleted from the helper vector and insertion of the target sequences were examined (FIG. 7). The sequences of the amplicons were determined (FIG. 8).

### Example 2

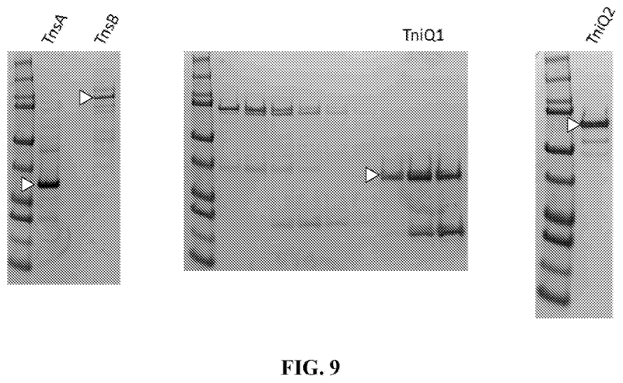

FIG. 9 shows purification of the proteins in CP000117: TnsA, TnsB, TniQ1, and TniQ2. Affinity purification (Strep-Tag) and size exclusion chromatography were performed.

Whether the system (CAST/homing to glmS) can be reconstituted in vitro was tested. Ion exchange chromatography can be used, e.g., to get improved purity.

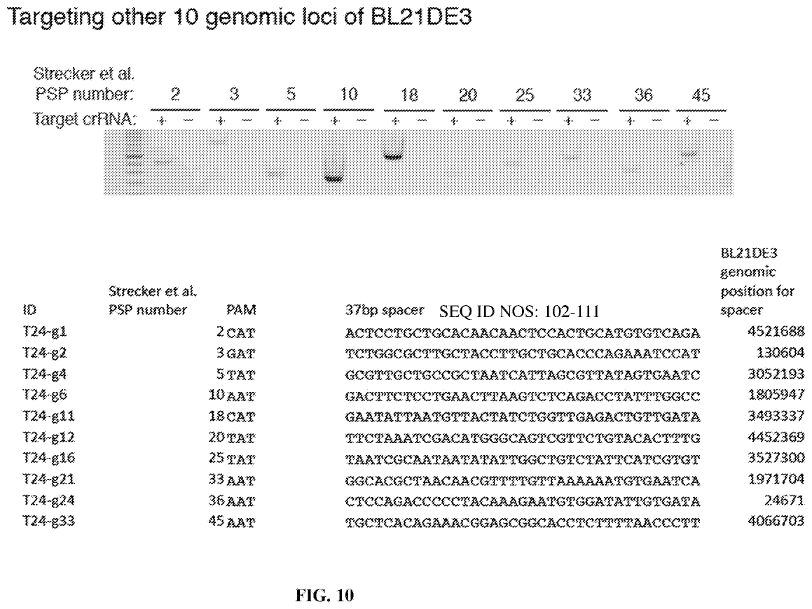

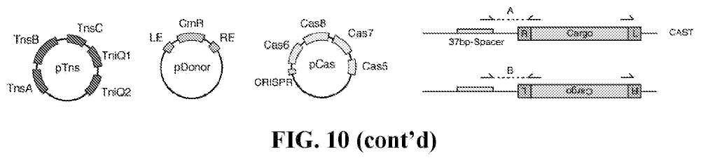

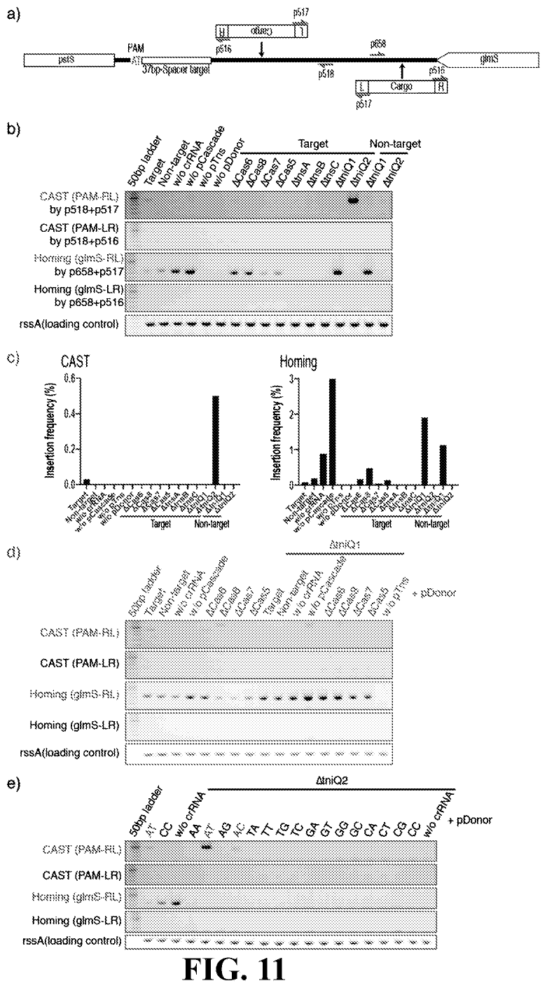

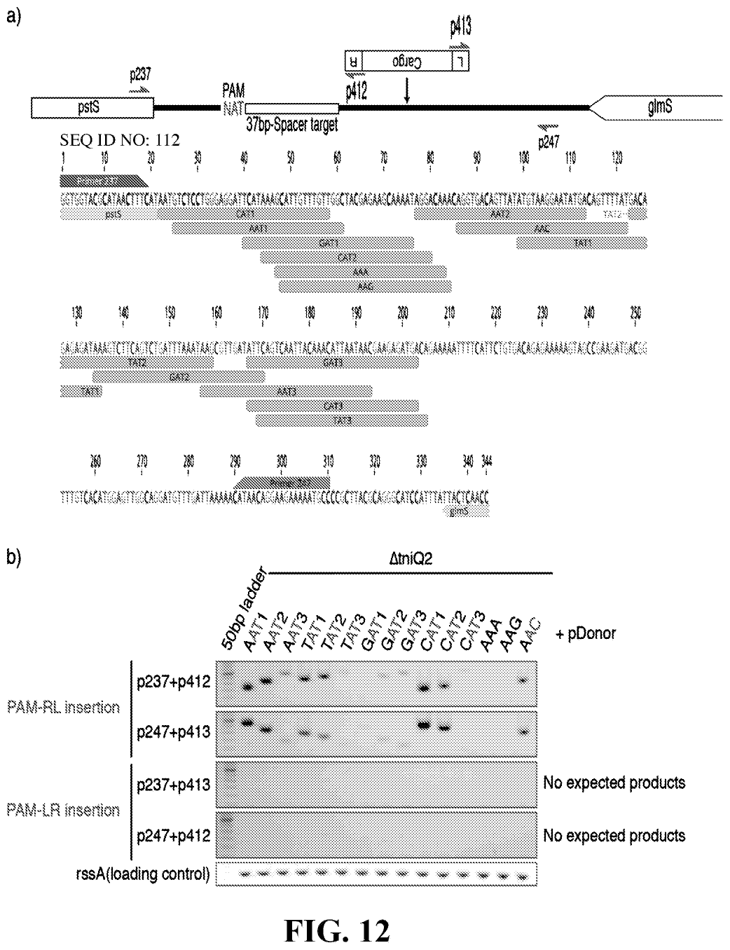

The system was tested for targeting 10 genomic loci of BL21DE3 (FIG. 10). The CP000117 CAST system locus harbors two TniQs for CAST and glmS homing (FIG. 11). FIG. 12 shows “N”AT-PAM tiling in glmS locus.

### Example 3—Biochemical Analysis of an Exemplary CAST 1-B System

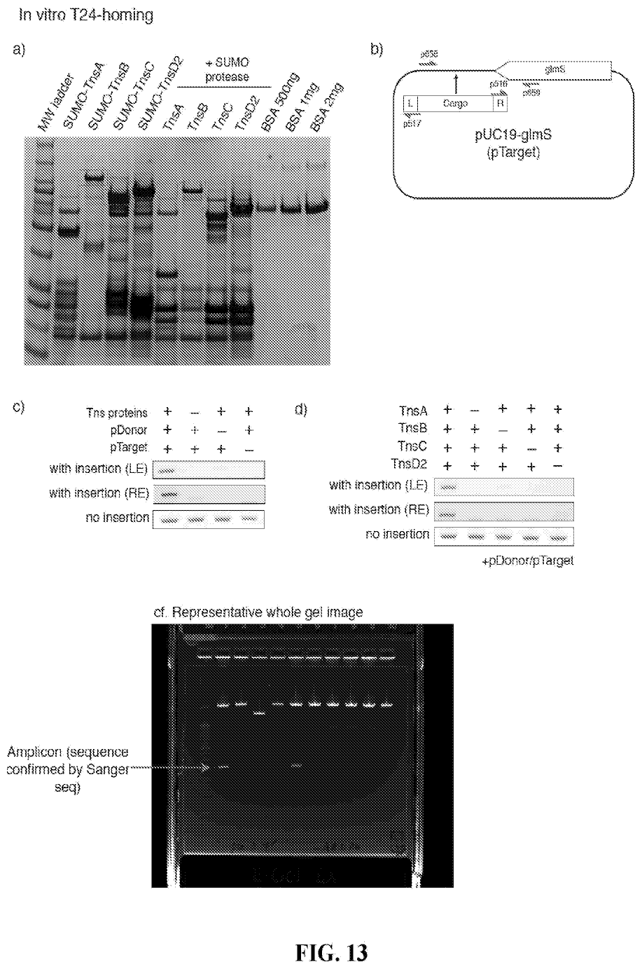

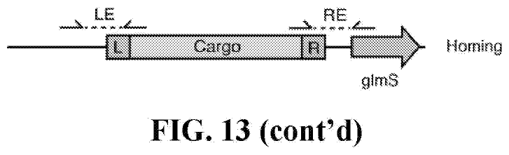

Fresh affinity purified SUMO-tagged TnsA, TnsB, TnsC, TnsD2 (+SUMO-protease cleaved native full-length TnsA, TnsB, TnsC, TnsD2) were used. Both tagged and native conditions worked. The T24-CAST may be reconstituted in vitro if D2 is replaced with Cascade+D1. The in vitro Tn7 reaction occurred at 200 mM NaCl. FIG. 13 shows the in vitro homing of T24. TniQ was capable of glmS targeted homing without Cascade.

Tn7 transposon was also tested in 293FT cells. Applicants transfected both N-terminal and C-terminal tagged (tagged with HA, NLS, linker) human codon optimized TnsA, TnsB, TnsC, TnsD2 in addition to pDonor and pTarget (glmS locus), which were used for in vitro assay. The result was robust with 3 independent transfection. FIG. 14 shows the homing of T24 in 293T cells.

For CAST, Applicants co-transfect FLAG-tagged Cas8 OR TnsD1 with other HA-tagged components and perform pull-down to determine whether they form complex. In parallel, Applicants purify D1-Cascade for RNP transfection. Applicants insert the glmS locus vector into the genome of 293FT by phiC31 integrase to see whether Tn7 happens on the mammalian genome.

### Example 4—Characterization of CAST 1B Expressed in Eukaryote Cells

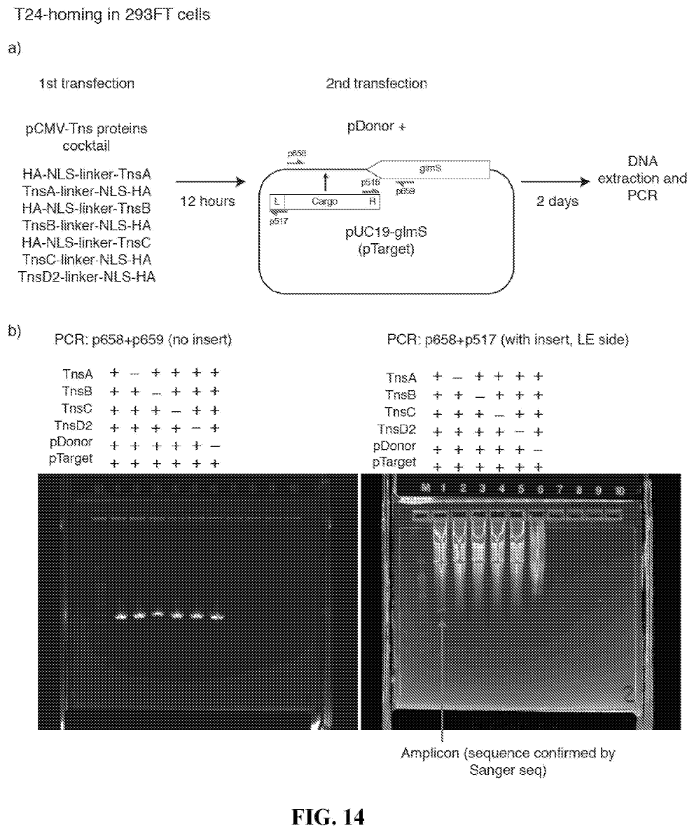

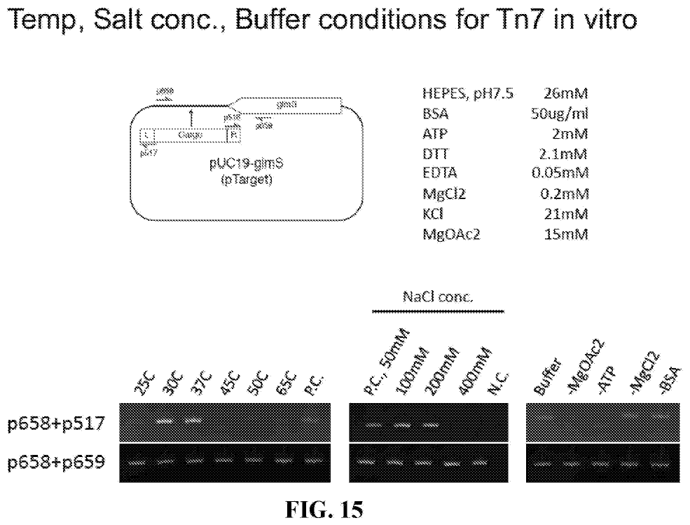

Applicants tested the conditions of temperatures, salt concentrations and buffer components for the activity and function of a CAST Type I-B system. In these experiments, CAST worked well at 30-37° C., at less than 200 mM of NaCl, and 15 mM of Mg₂, and 2 mM of ATP. The results are shown in FIG. 15.

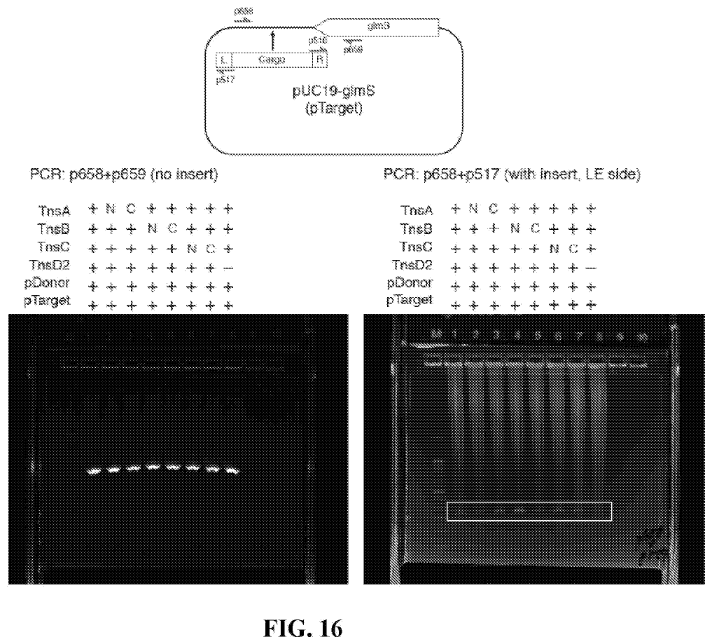

Tags were fused to the N- and/or C-terminus of the CAST plasmids. Insertion of a donor polynucleotide by CAST was tested. The results are shown in FIG. 16.

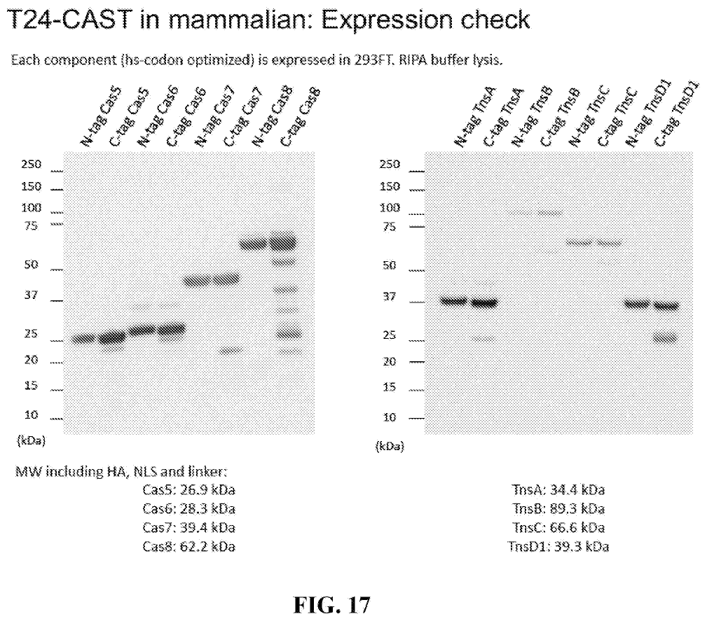

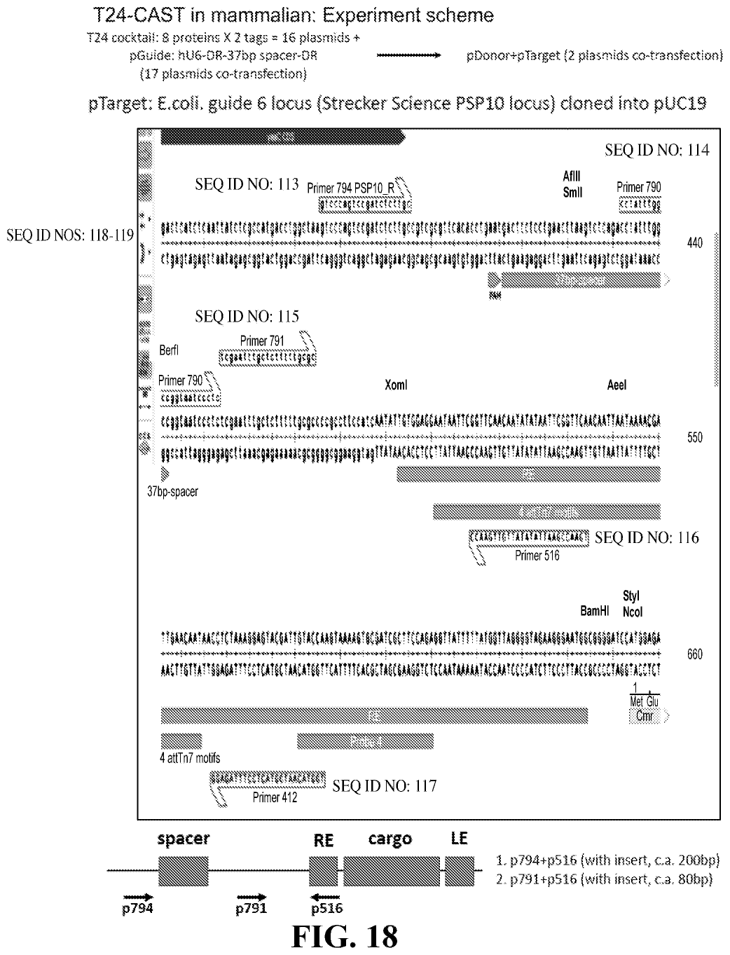

The components of CAST were codon optimized for expressing in human cells. Expression of the components were tested in 293 cells. HA tag was used to detect expression. The results are shown in FIG. 17. The plasmids used in testing the expression in 293 cells are shown in FIG. 18.

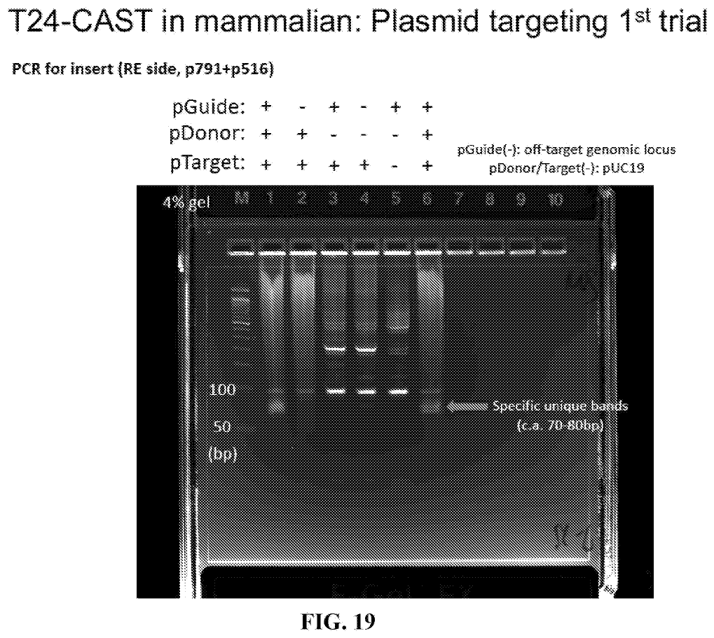

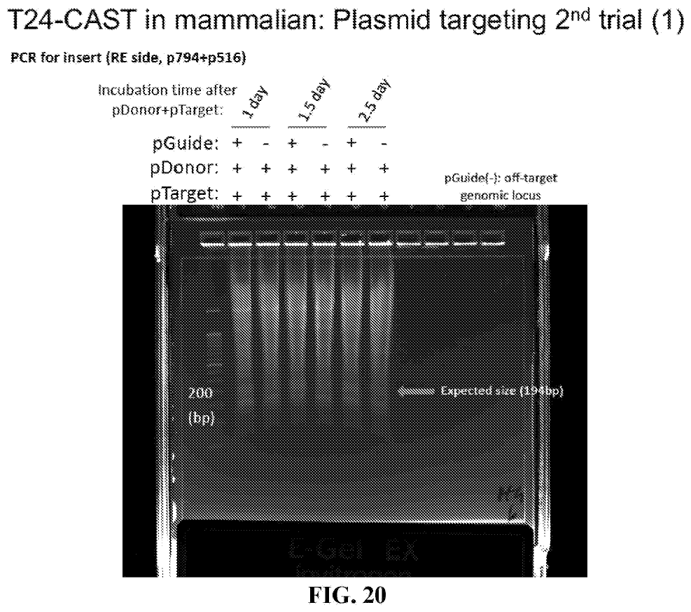

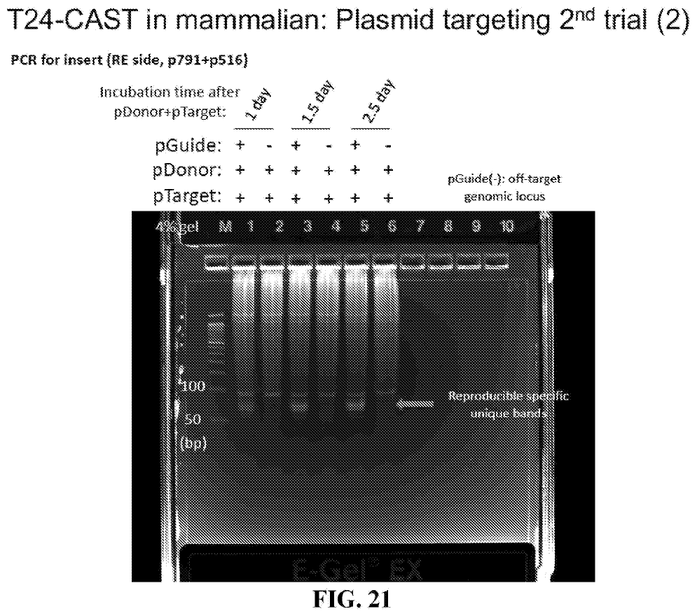

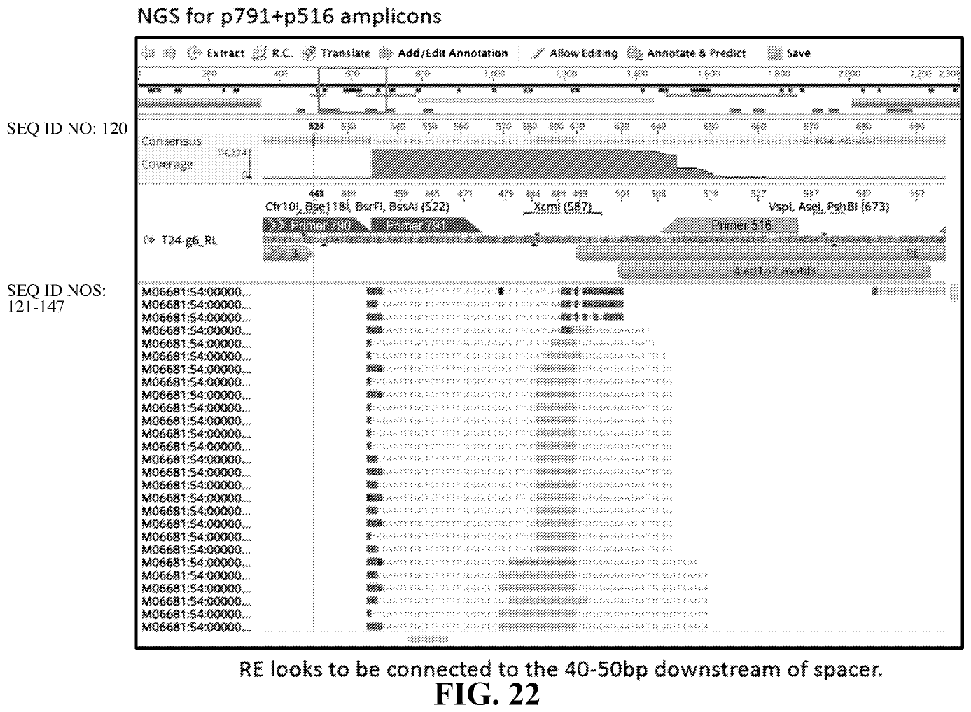

Insertion of donor polynucleotides at target sequences by CAST in mammalian cells were tested (FIGS. 19-21). The insertions were characterized by next generation sequencing (FIG. 22).

### Example 5—Characterization of Functions of TniQ1 and TniQ2

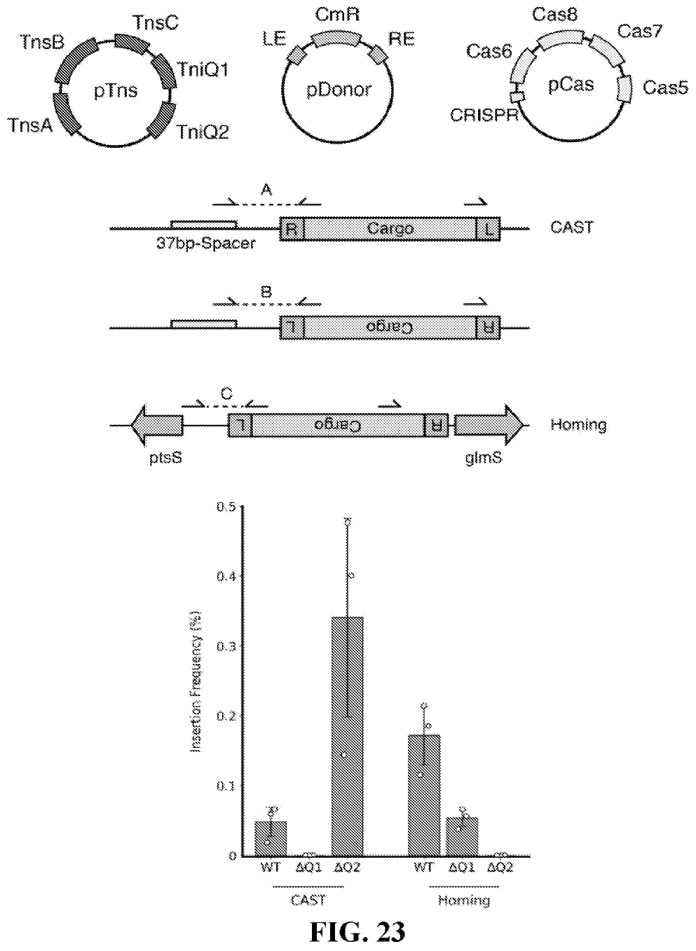

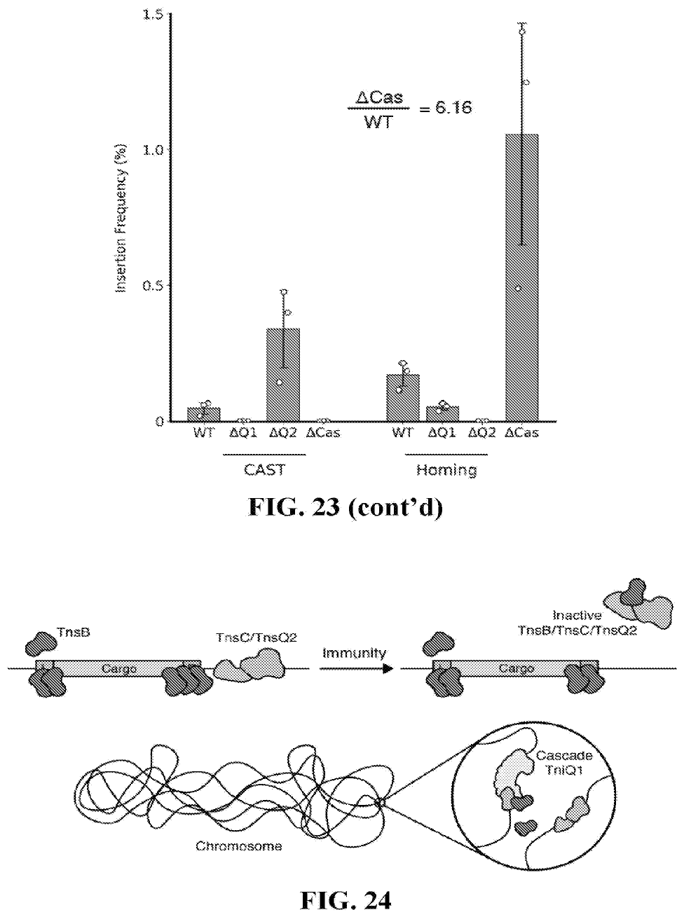

Roles of TniQ1 and TniQ2 were tested. In these experiments, TniQ1 was needed for the insertion of the donor polynucleotide to target sites (guided by guide RNA) by CAST, while TniQ2 was needed for homing to a specific target site (FIG. 23). CAST and homing transposition completed for shared components (e.g., TniQ1 and TniQ2).

### Example 6—TnsB Target Immunity Interactions Suppress Transposition

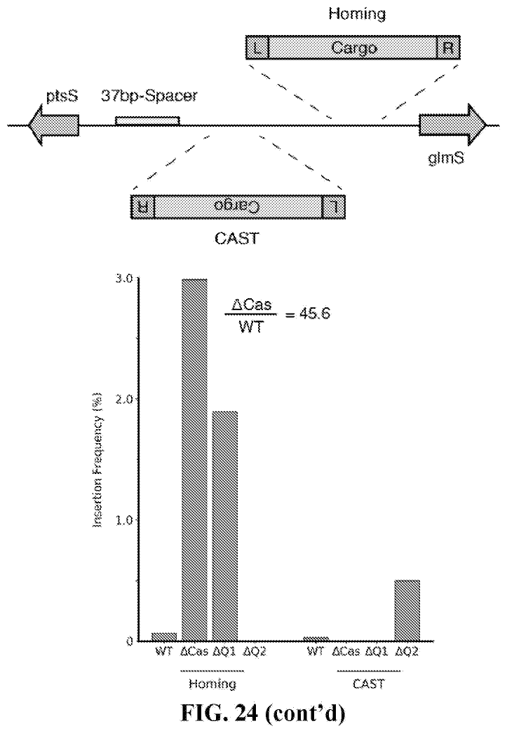

Function of TnsB and CAST may be related to the ratio between the levels of TnsB and other components in the CAST system (FIG. 24).

### Example 7—Characterizing Type I-B CAST: PAM Screening

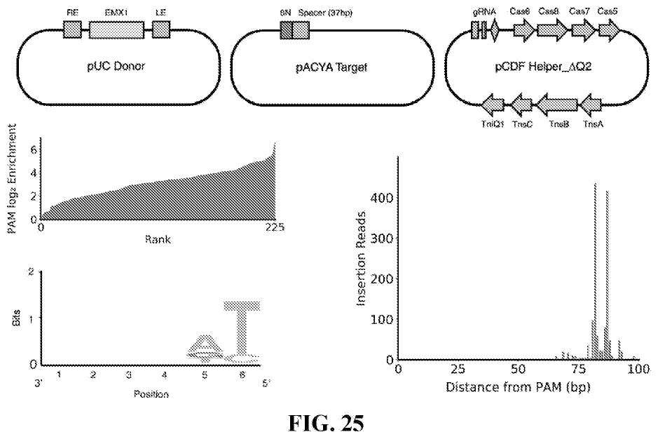

PAM motifs were screened for exemplary Type I-B CAST (FIG. 25).

### Example 8—Characterization of TnsB

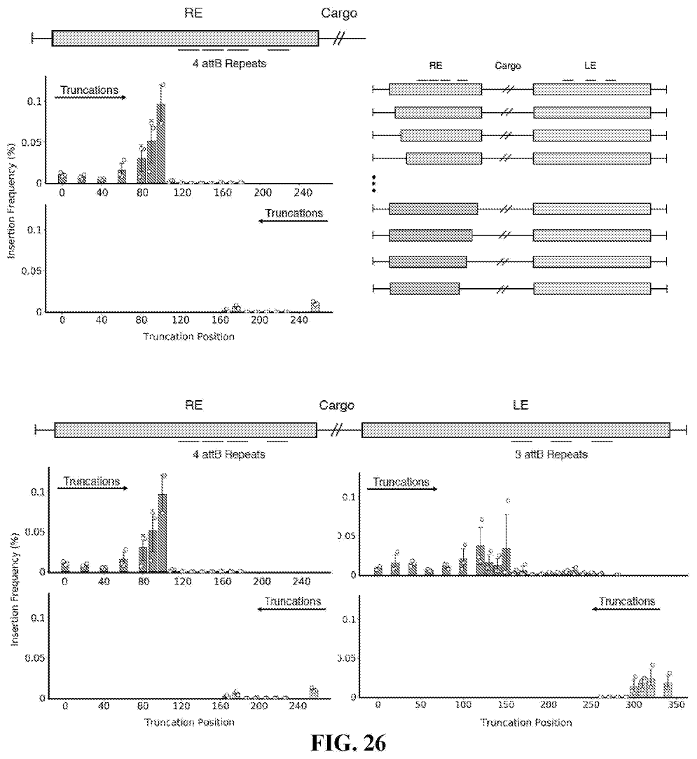

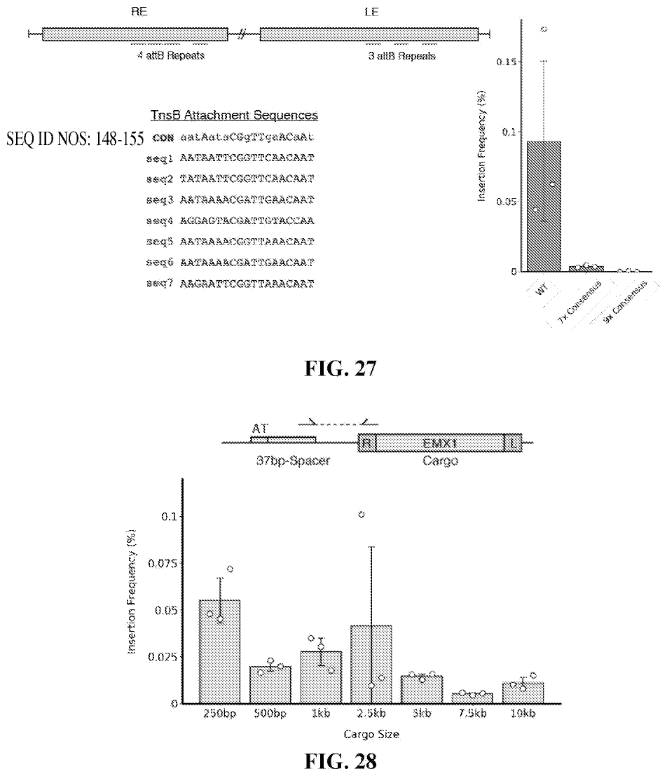

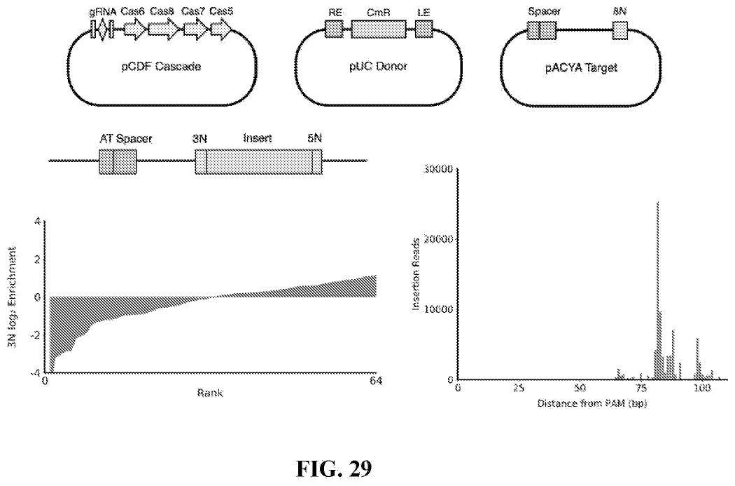

Minimal TnsB binding sites were examined (FIG. 26). The TnsB consensus sites were investigated (FIG. 27). Various cargo sizes were tested (FIG. 28). Insertion site specificity analysis was performed (FIG. 29).

### Example 9—Characterization of an Exemplary CAST

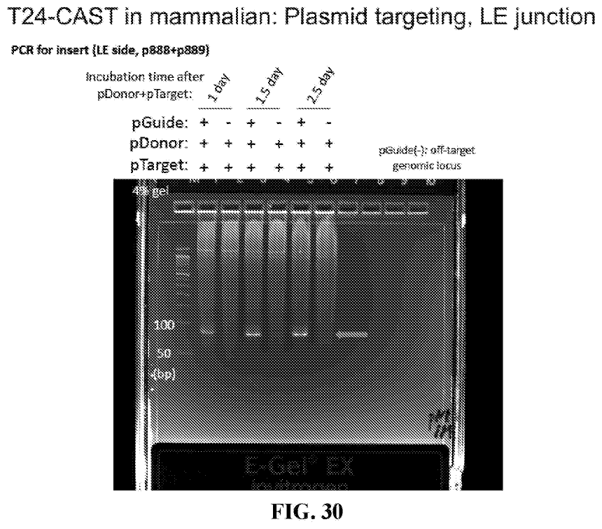

FIG. 30 shows the results of plasmid targeting and LE junction of an exemplary CAST in mammalian cells.

### Example 10—Screening PAM Sequences in Mammalian Cells

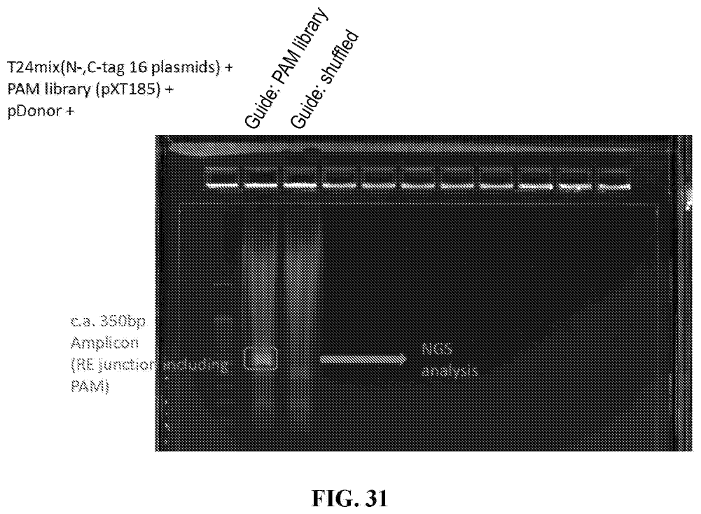

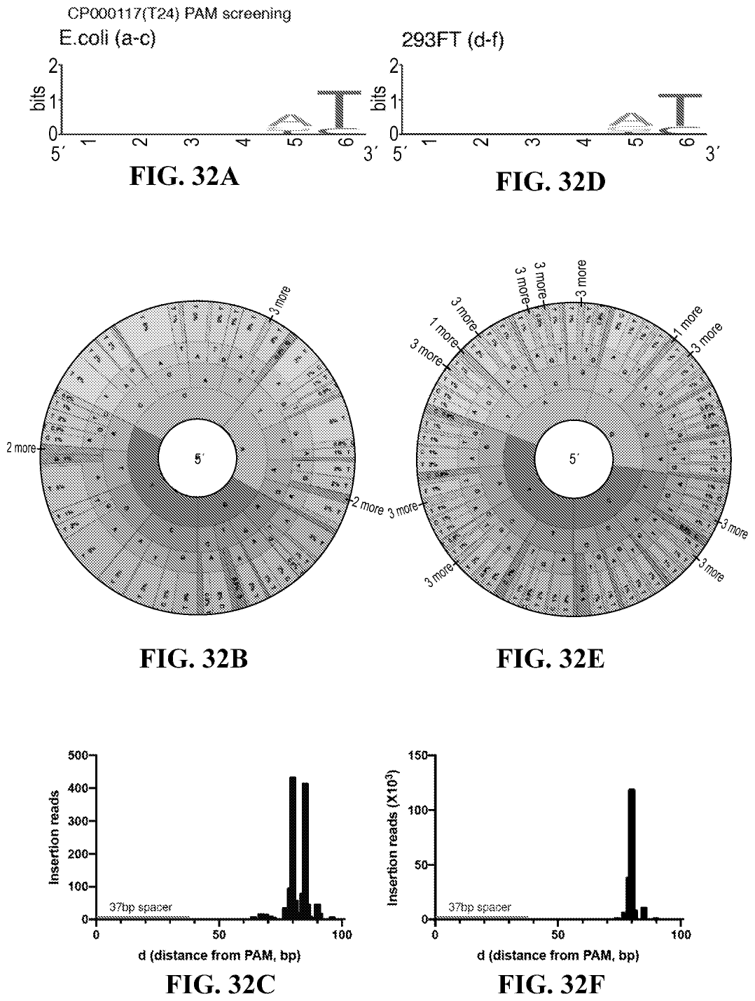

Applicants performed PAM screening of exemplary CAST systems in 293FT cells. FIG. 31 shows the analysis of plasmids used in the screening and FIG. 32 shows the screening results.

### Example 11—Analysis of Functions of TnsD1 and TnsD2 in Mammalian Cells

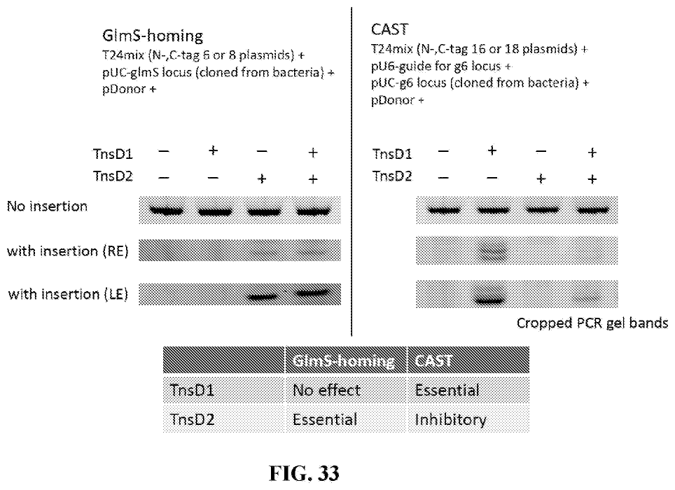

Applicants tested the competitive and/or cooperative effects of TnsD1 and TnsD2 on homing and CAST functions. Insertion of target sequences were tested with the presence of TnsD1 and/or TnsD2. FIG. 33 shows the result of the analysis.

### Example 12—Examination of Plasmid Targeting in Mammalian Cells

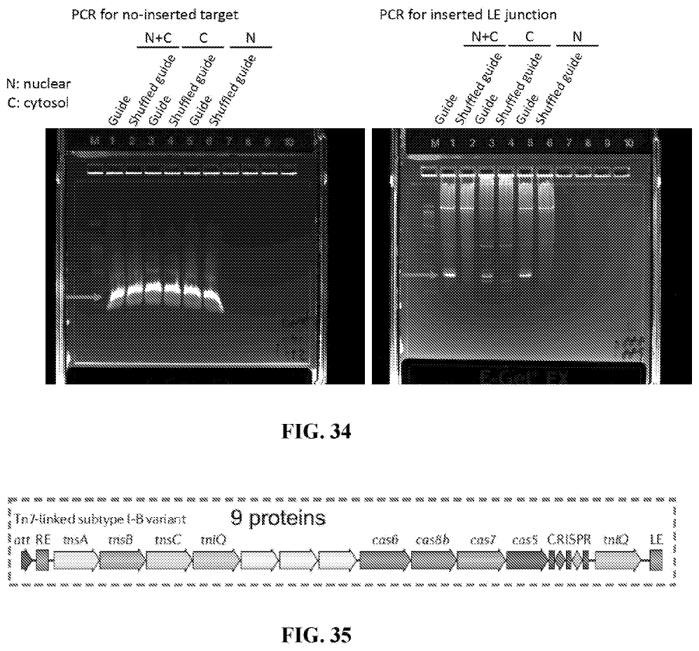

Applicants examined the subcellular location where the plasmid targeting happened. Transfected plasmids were separated from nuclear and cytosol fractions. FIG. 34 shows the results, which suggest that the plasmid targeting happened in nucleus.

### Example 13

FIG. 35 shows exemplary Type I-B CAST systems.

### Example 14

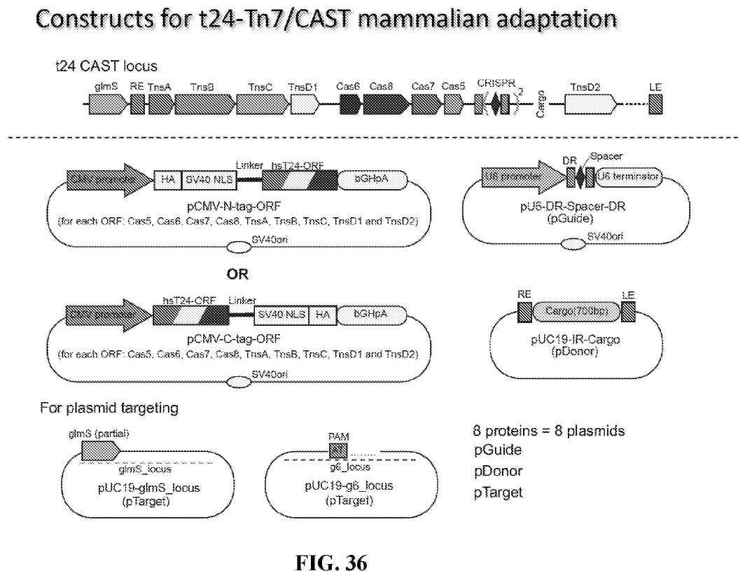

T24 CAST locus is shown in FIG. 36. Constructs for adapting the T24 CAST system in mammalian cells are also shown in FIG. 36.

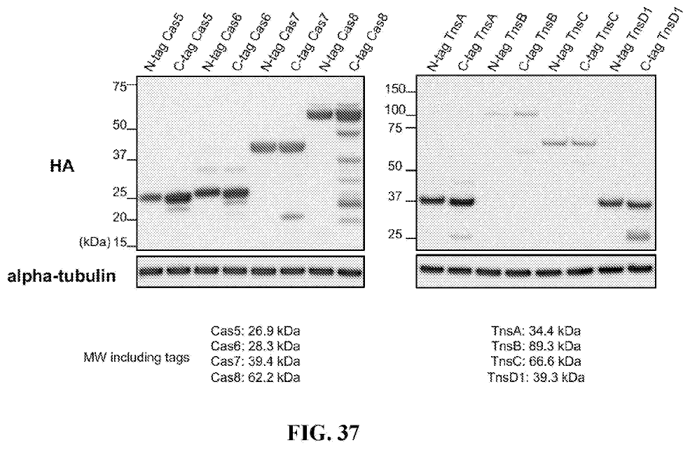

Expression levels of the components of the T24-CAST system in mammalian cells were tested. The components were transiently expressed in HEK293FT cells. The cells were lysed in RIPA buffer, and 30 μg protein was loaded on each lane for analysis. The results are shown in FIG. 37.

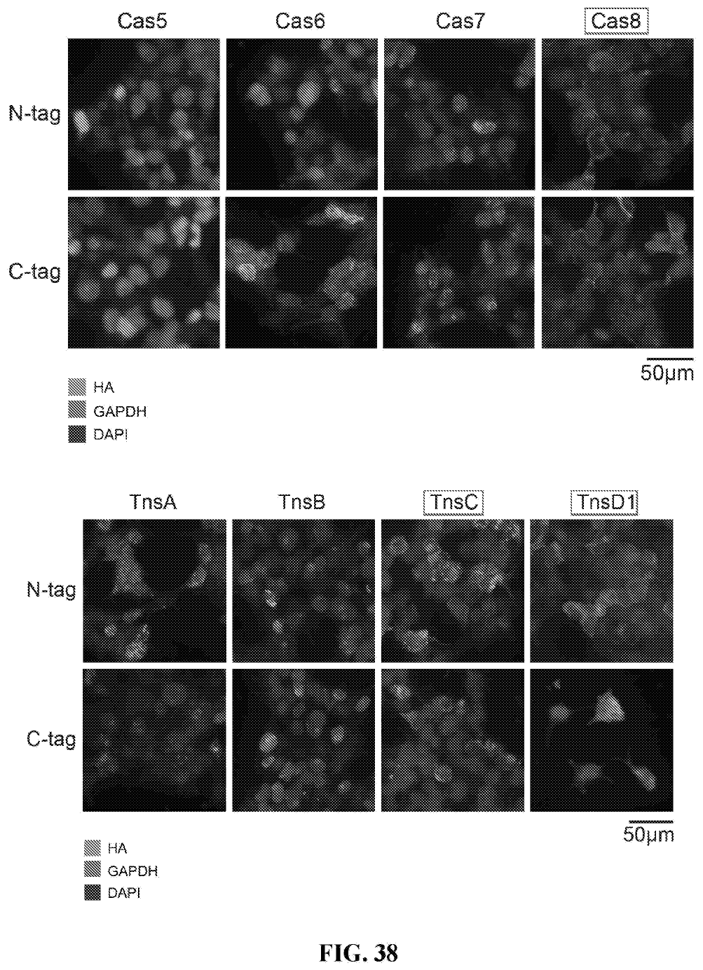

The components of T24-CAST system were tagged with NLS. Localization of the NLS-tagged components were examined (FIG. 38).

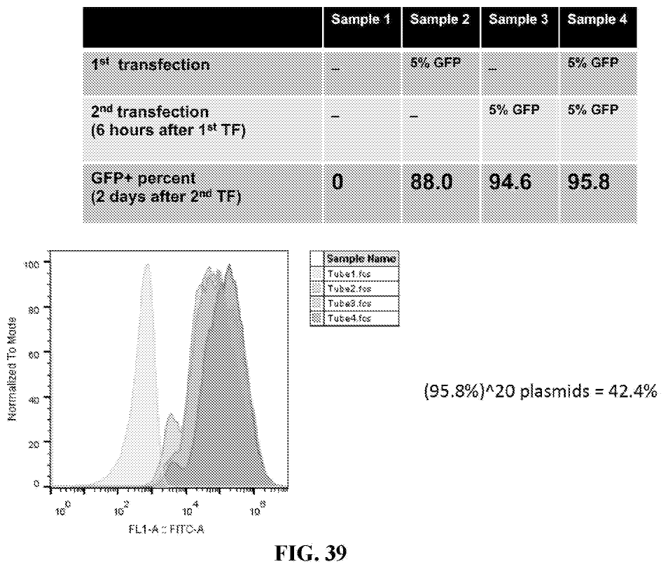

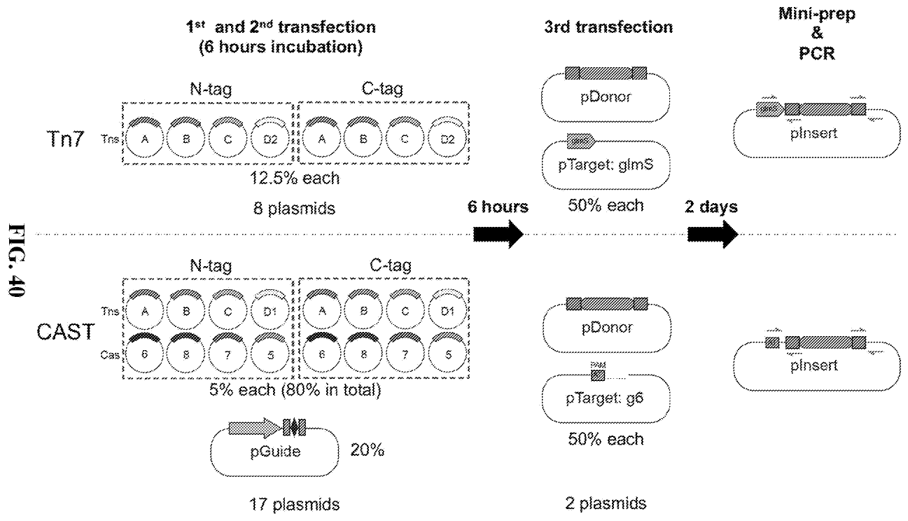

GFP was spiked-in transfected with lipofectamine3000 and the result was shown in FIG. 39.

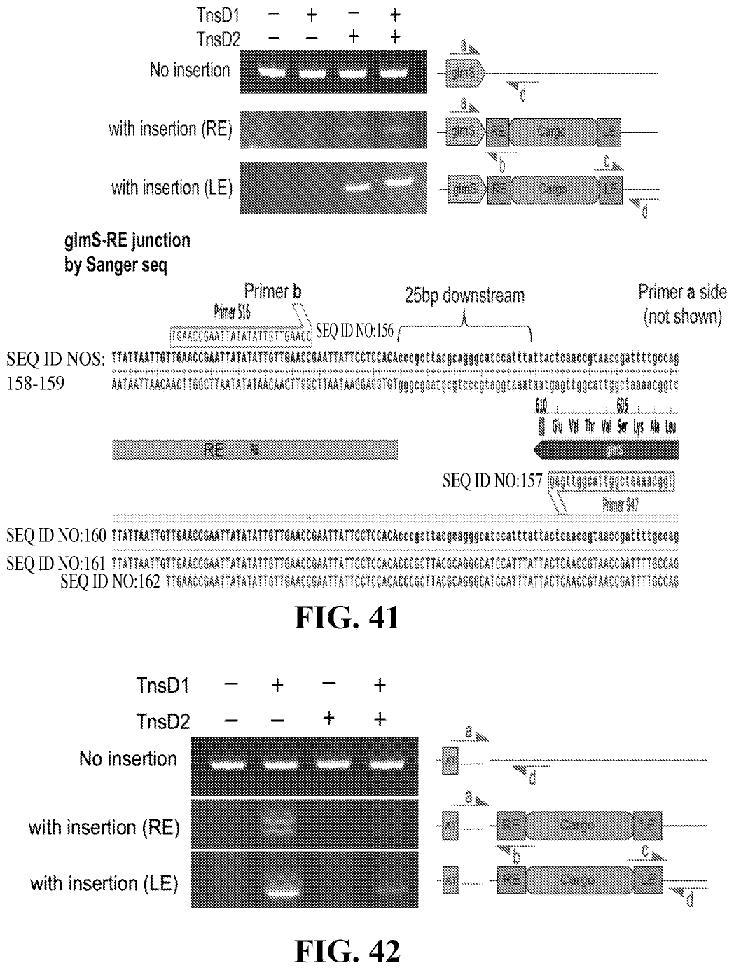

FIG. 40 shows experiment schemes for analyzing Tn7/CAST plasmid targeting 293FT cells. The plasmid targeting of T24-Tn7 system in 293FT cells is shown in FIG. 41. The plasmid targeting of T24-CAST system in 293FT cells is shown in FIG. 42.

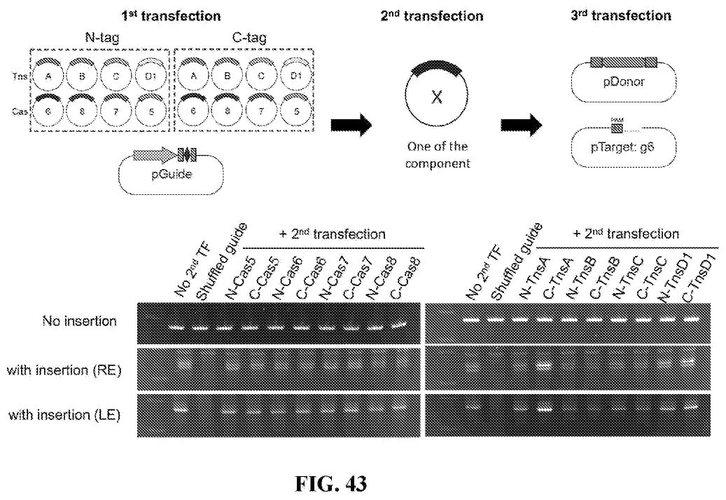

Roles of individual components in T24-CAST were tested by transfecting selected components in the 2nd transfection and tested the effects. The results are shown in FIG. 43.

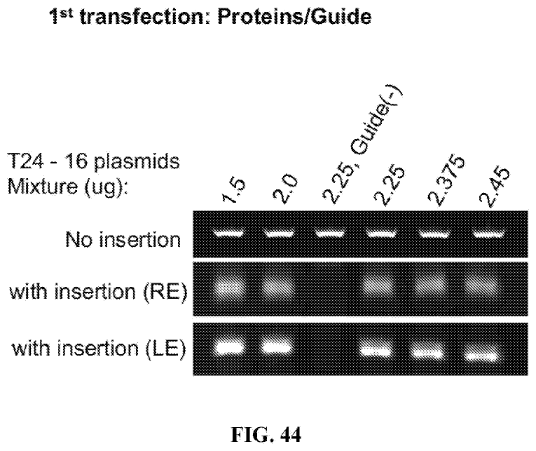

The ratios between proteins and guide molecule, and the ratios between donor and target were optimized (FIG. 44). In some cases, less protein expression was desired.

### Example 15

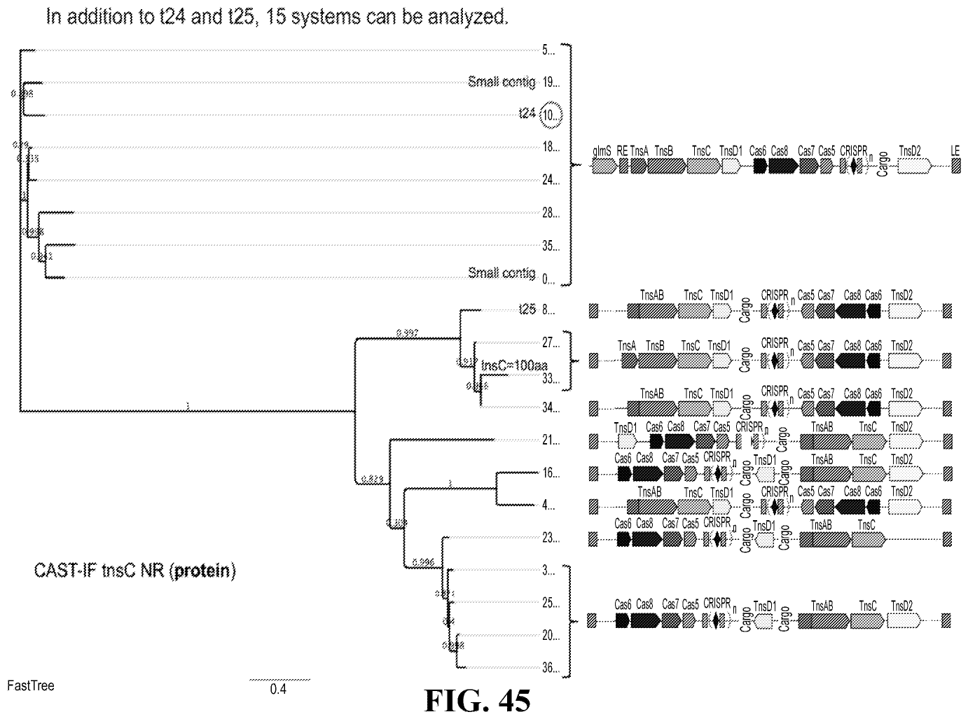

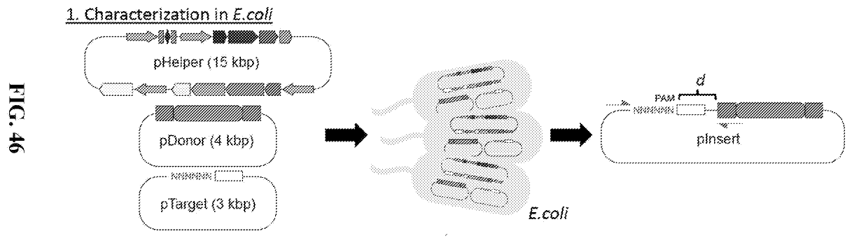

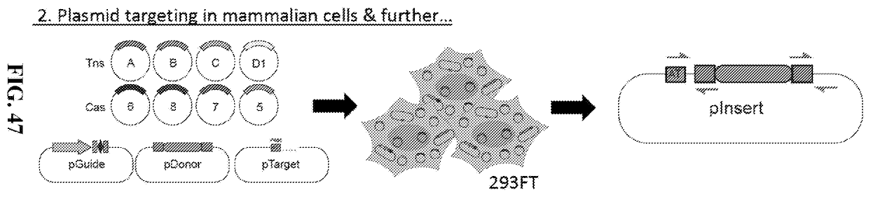

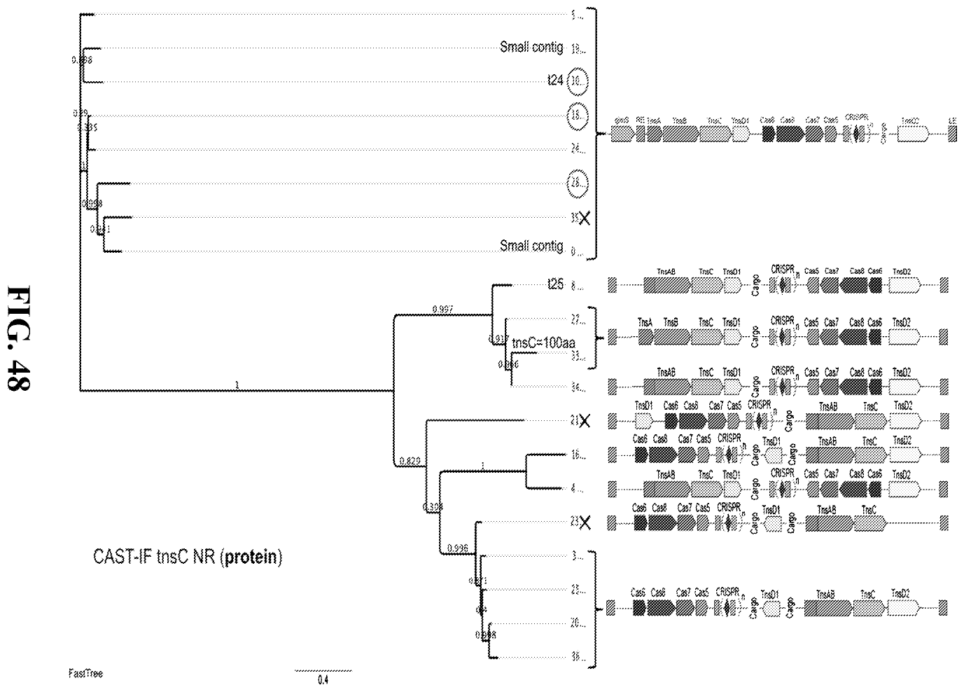

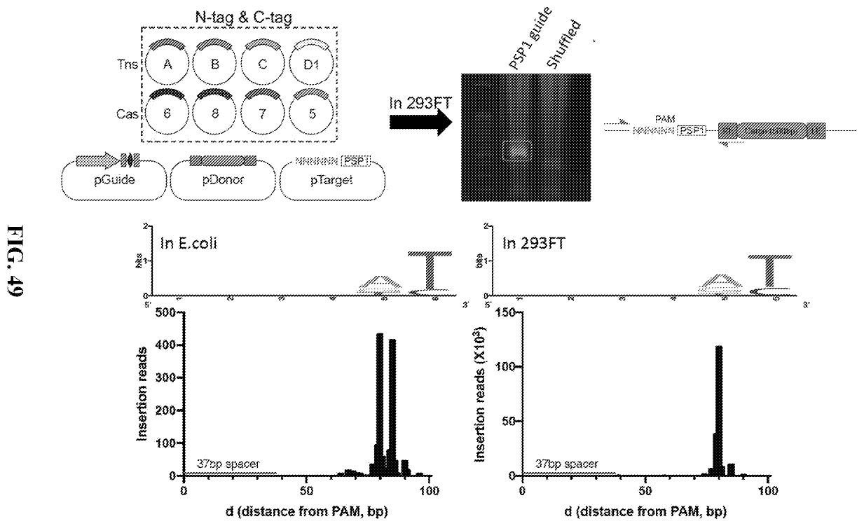

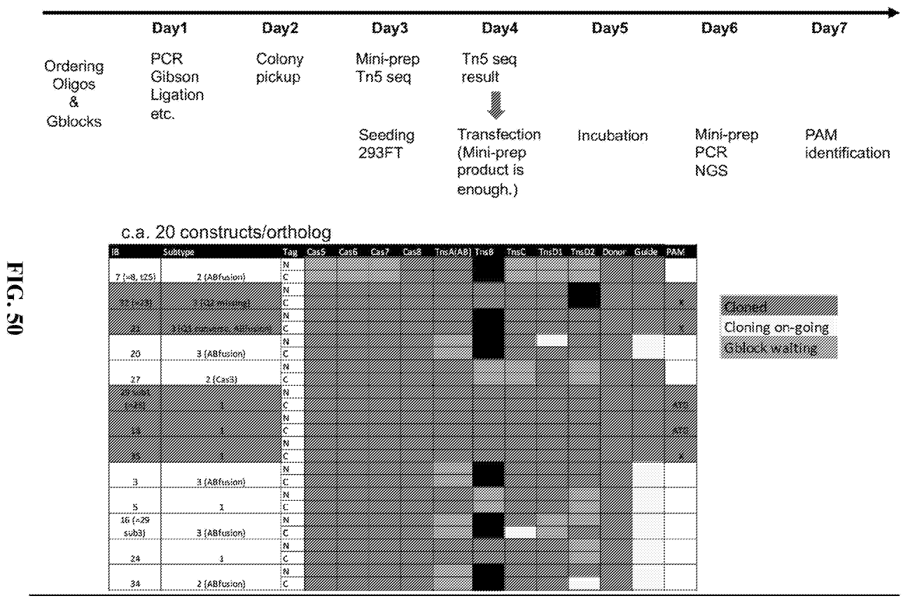

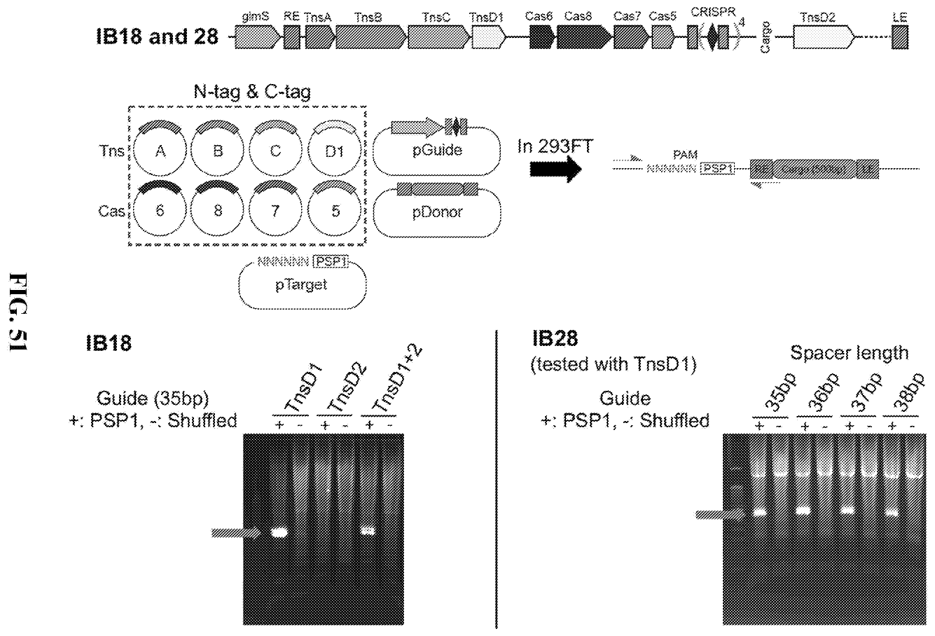

Additional Type I-B CAST systems are shown in FIG. 45. Additional Type I-B CAST systems are analyzed in E coli (FIG. 46). Larger pHelper construction may be performed. Human codon optimized proteins may be expressed in bacteria. The plasmid targeting profiles are analyzed in mammalian cells (FIG. 47). Different expression vectors may be used. Three Type I-B CAST systems were tested to show work in mammalian plasmid targeting (FIG. 48).

### Example 16—PAM Screening for T24 in 293FT Cells

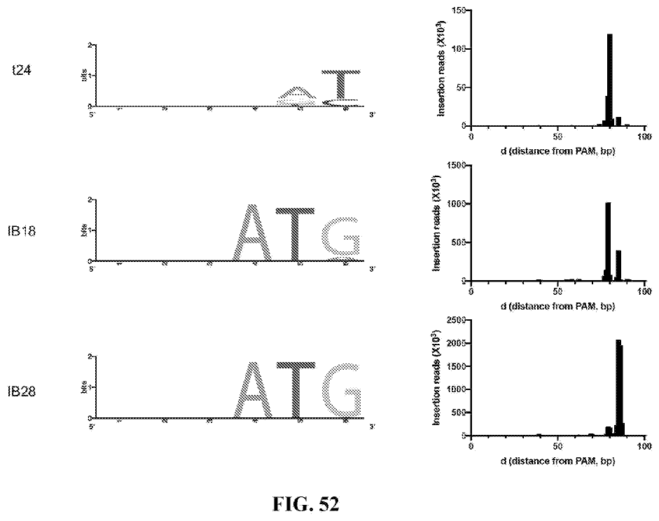

PAM sequences for T24 were screened in 293FT cells (FIG. 49). FIG. 50 shows mammalian PAM screening pipelines for new orthologs. For 2 new orthologs (IB18 and IB28), PAM libraries were generated and plasmid targeting was tested in 293FT cells (FIG. 51). PAM screening results for 2 new orthologs (IB18 and IB28) are shown in FIG. 52.

### Example 17—Sequences of Exemplary Type I-B CAST IB18 and IB28

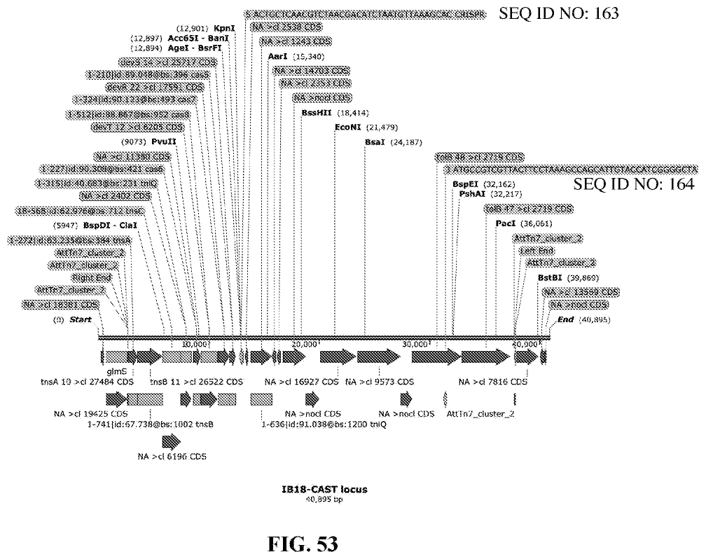

The map of IB18 locus is shown in FIG. 53. The sequence of IB18 locus is shown below.

| IB18-CAST locus (40,895 bp); Accession: | |
|---|---|
| 100521\|34\|GCA_001597855.1_ASM159785v1_genomic\|LUHI01000035.1\|201628\|Anabaena | |
| (SEQ ID NO: 65) | |
| 1 | ggttttgaat tcaagtaacg cttcatatat gtttacaaat cggctaaatc tctgattggg |
| 61 | tgatgaggag aaatacgagg cggaaatgtg gctattttat aggaacagag agagattgcc |
| 121 | gaaaagtgaa ttttgagaag tgcctattaa attgtttaaa gttatgtagc cttttactta |
| 181 | cgccgtataa gccactgcgg taaactatgc gttgattatg attctttcac agataaagaa |
| 241 | cactggaaag gagccgtttt ttcaatgtct cataccgtaa aaatctacga tacctgcatt |
| 301 | ggctgcaccc aatgtgtccg cgcttgcccg actgacgttc tggagatggt tccttgggat |
| 361 | ggctgcaaag ctgctcaagt tgcttcttcc ccccgtacag aagactgtgt aggctgcaaa |
| 421 | cgttgtgaaa ctgcttgccc caccgacttt ttgagcatcc gggtttacct gggcgcagaa |
| 481 | acgactcgca gtatgggtct agcttactaa ggaattaact gctaattcct gatttagttg |
| 541 | accattcagt gcctcgatag caaacaactt tagcattgta gggtgggcgg cgatcgctca |
| 601 | tcctacaatg tcaaactgga aaaatatgaa tatacttctt gacacagggg ctaacagctc |
| 661 | ctttttttgt ggaactcgat atttagatca gcatctatac ttgatgtaat tatcctgaac |
| 721 | aggtggtgtg agtaatgtgt ggcatcgttg ggtatatagg cactcaaaca gcgacagaaa |
| 781 | ttttattagc agggttagaa aaactagagt atcggggata tgactccgcc ggaattgcca |
| 841 | cggtttggga aggagaggtt aattgtgtcc gcgctaaagg gaaactctac aatcttcgtt |
| 901 | ctaagctgga acagtcagca acaccagccc aaataggcat cggacacaca cgctgggcaa |
| 961 | ctcatggtaa accagaggaa cataacgccc atccgcattt agacacagct atgcgggttg |
| 1021 | ctgttgttca aaatggaatt attgaaaact accgcgagtt acgcgaggaa ctgaaacaga |
| 1081 | aggggcatca gttccgttcg caaaccgata cagaagtgat tccccatcta attgcagaat |
| 1141 | atttacagca tcttccctca ccttctcacc ccctcacccc ctctgacttt ttagaggcag |
| 1201 | tgcgccaagc tgccaatcat ttagaagggg catttgcgat cgccgtcatt tcggctgact |
| 1261 | accccgacga gttggttgtg gtgcgtcagc aagccccctt agtgatagga tttggtcaag |
| 1321 | gtgagttttt ctgtgcttct gataccccgg caattgtcgc ctatacccgc gccgtactcc |
| 1381 | ccctagagaa tggcgaaata gcccgcctca cacctttggg agtggaaatt tacaactttg |
| 1441 | ctggcgacag gttgaaaaga caaccccgaa tgctcaactt gaatcccacg atggtagaaa |
| 1501 | agcagggatt caaacacttc atgctcaaag aaatttatga gcaaccggga gtagtcagag |
| 1561 | ctagtttaga agcatatttt gatcatgata ccgttaacct agacttacca caggattttt |
| 1621 | acgcagattt agagcaaatt cacatcgtcg cctgtggtac aagttggcac gctgccttag |
| 1681 | tgggtaaaca cttactcgaa caactagcgg gaatttctac ccaggtacat tacgcttctg |
| 1741 | agtatcgcta tgccccatca cccctgacag ccaatacctt aattattggt gtcactcagt |
| 1801 | caggggaaac ggctgatacc ctagcggcgt tagcaatgga aaaagaacgc cgccaaggaa |
| 1861 | aagaagcgaa atatcaggcg cgacttttgg ggattaccaa ccgcccagaa agtagcctcg |
| 1921 | gtttgatggt tccccatatt attaataccc tagccggaat tgagattggg gtagcggcga |
| 1981 | caaaaacctt tattgcccag ttgatggcgt tttatgcctt agcattggat ttagcggctc |
| 2041 | gtcgccagac actaccaccg gaaaaactca cagaaattat tcaaggcttg cgccagattc |
| 2101 | ccaaggaaat tgaggcgaca ttagaacgtc aggaagaaat caccgaacac ttagcccatg |
| 2161 | aatttgcgga aaccaaagat tttatctttt tgggtagagg gattaacttc ccgatagcat |
| 2221 | tagaaggggc gttgaaatta aaagaaatca gttatattca cgctgaaggt tatccggctg |
| 2281 | gggaaatgaa acacggcccg atagctttgt tagatgctaa agttccggta gtagcgatcg |
| 2341 | cagttcctgg tactgtgtat gaaaaggtga tttctaacgc tcaagaagcc aaagccagag |
| 2401 | attcccggtt aattggggtg actcctgtga cagatggcga agccgcagaa atctttaacg |
| 2461 | atttactccc agtgtcatca gtggatgaat tattatctcc catcctgaca gtcgtacctt |
| 2521 | tgcaactttt ggcttatcat attgcagcaa gaaggggttt agacgttgac cagcctcgga |
| 2581 | atttggcgaa gtcggtaact gtggagtaaa ataatacttt aaataagtat aattgtacaa |
| 2641 | caataaaaaa gtcgtataac aaataaaaaa gttgtacaat atataaaaaa gttgtacaat |
| 2701 | atatcctgtg ggtgcatatg caagcatcta caggagtttt tatggctaga agtaagcaag |
| 2761 | attggacgca agccaagttt gagcgttact gtaaggaagg tcgtggtcag ggcagtggga |
| 2821 | aaaactatca gccctggatc aagattcagg actttccctc caagggtcgt gtttccagac |
| 2881 | caccaggttg gaaaacaaac cgagaacatc atctcttttc tgataacgag aagcgactgt |
| 2941 | tttacgtatt tgagtggtca gatgcgatcg ttgatattag agagcagttt cctctacttg |
| 3001 | atctggactt agcaattaat attgcggatg aaatgggtat taactatccc aaagatccgc |
| 3061 | aaagcaatac gccatatgtt ttagccacgg attttatgct ttctgtccag caaggtaaga |
| 3121 | atatagttca gaaagcacga acatttaaac taaccaagga tttgggaagt aagtcggtag |
| 3181 | ctgaaaaatt tgagttagaa aaacgttact ttgctgccaa aggtattgat tggggaataa |
| 3241 | tcacagaaaa agaagtccct aaacaattag ctgagaatgt tgaatggatt catactgctt |
| 3301 | ataaattaga agaaaatgca gatataaatc ttgaagaatt acgtaacatc gcaaatattt |
| 3361 | taaagtttag acttcaggaa agtgatgcaa gtatcaataa aataacaacg gttttagata |
| 3421 | aggagatgaa catagagtct ggcacatctc tctatctttt tagacacctt attgctcgga |
| 3481 | aagaaatcat tatggatatg ctggtaaata aaatatctag ttgtccatct tctaaagaaa |
| 3541 | ttcaaaaaat aattttttaa gctaggacga ggtactttat atgattaatg atttgtttgt |
| 3601 | aaatgacctg attgaatgga ttgatgaatc cggtaataac tttatagaac gagttctctg |
| 3661 | gattgatgaa ggatatatta ttgcctttgt ttatgatata aatgctaaaa caggatttcc |
| 3721 | agaggctaaa aaagtttcag agattagaga agctatttct gatggtcgtg cattgaagct |
| 3781 | taaatcagac ccgtgggcaa gaatcgtaag agatgaagat ttgtcagaga aagaaaaaga |
| 3841 | acttagagat agggcttggc aaataatttc atttatcgtt actcaagaac cctcaatata |
| 3901 | ctatcgggat tatagaggta ctttagttca gcaggttatt gaaaaatata atgcaggtag |
| 3961 | aactgaaggt gaattagtac caataactgt ttataaatat ttaagaagat tttggcagag |
| 4021 | gggtaaaaac aagaatgctc tcttgcctga ttatgcaaat tctggtggta aaggaaagcc |
| 4081 | taaatcttct ggagacaaga agagaggaag acctagaaaa tacgcacaag ttccagaaat |
| 4141 | tggtgttgga agaaatgtta cagaagaaga taaaagaata tttagatttg caactgctaa |
| 4201 | attctataat aattccaaag aaaatttctt aacgactgct tatgatttaa tgattaaaga |
| 4261 | atattacgct gaagattttt attacgatga taatggagtt aagaaaagta ttttaattcc |
| 4321 | tccagataaa cgacctacat ttacccaatt taaatactgg tacgaagtag aacaaactga |
| 4381 | tatcagaaaa actatgatat ctcgtaaggg ttcgagaaaa tatgccttag aaaatcgagc |
| 4441 | cattaccgga acttcccaaa tggaaactat tgggccaggg tctagatatc aaattgatgc |
| 4501 | tacgattgca gatgtatact tagtatccct ttacaaccgc aactggatta ttggtagacc |
| 4561 | tgttatatat gtggtgattg atgtttttag tcgcatgata acaggggttt acattggatt |
| 4621 | ggaagggcca tcttggacag gagcaatgat ggctttagcc aatgcagcta cggagaaggt |
| 4681 | caagttttgt cgggagtatg gtattgagat ttctgaagat gagtggccat gcaagcatat |
| 4741 | tcctgatacc attttaggcg acagaggtga attagcaggg atgacagtag aaacattaat |
| 4801 | ccctaattta aatgttcgta ttgaaaatac tgctccttat cgagcagatt ggaaaggatt |
| 4861 | agtggaaaga caatttcgca ttattcatgg gtatgtcaaa ccatttgttc ctggttatat |
| 4921 | agatactgac tttagacaga gaggaggaca tgattatcgc cttgatggta ggcttaacat |
| 4981 | agatgaattt actaaaatag ttatattttt gattctccag cataacaatc atgattatct |
| 5041 | aattgattat gctagagatg aaatgatgat taccgatgat gttaatccta tacctagaga |
| 5101 | attatggcaa tggggaattg ccaatcgttc tggtagactg agaacttttc cagaagatat |
| 5161 | tgtcaagcta aatttaatgc ccacagaaaa agcaacaatt actgctcgtg gtatcaaatt |
| 5221 | taaaggaatg tactacactt gcgagaaagc aaggaaggaa ttttggtttg aaaaatctag |
| 5281 | aagtagttta ttatctaagt cagagaaatc attgaatata tcatatgata ttcgacagcc |
| 5341 | aaactttatt tatttacgtt ccccaaacgg tagagaattt gaaaaaggtt ggcttttaga |
| 5401 | atcgcaacaa agatatttag ataaaaactt ttatgacatt gattatttat tagcatatga |
| 5461 | aaaattgcaa aatcaaaaac atcaaggtac tcggttacag gcaaaagctg atctgatagc |
| 5521 | caatatagaa agtattgtta gtcaggctaa agaagagact gaggcagttt tagatgatac |
| 5581 | tataagtaat agacaaaaag ttagtggcat tcgacagaat agagcagacg aaaaaaatgc |
| 5641 | tcgacgtaaa aatgaagcat ttgaactagc aaaagcagaa actcccaata ctgataatac |
| 5701 | taagaatcta ataaacactc aatctgaaga ttccgaggat tcaaaactat tacagcctga |
| 5761 | ccatatggat ttactcaaac ggaaaagaca ggagcgaaaa cgtggaaagt aatgaggaag |
| 5821 | aagttgttag tattcctaat ggaaagtcag cagttattgc cagatacata gaccaaaaac |
| 5881 | ttccagaata taacgctaat ccattgattc aggcactacc tcctatcctc tcagcagaag |
| 5941 | aatttatcga taaagtaacg agaacaccag attttgatga acaagagagg gagctagaag |
| 6001 | cgcactacag gtttcattgc attgagaggc tatctagata ttttgatcct caaaataaaa |
| 6061 | ctgttgaact acagaaggtc atttgtgcgc taattatgac gggctattta gctcggaata |
| 6121 | ttctgaaacc tgaatatgca agtcgctcta ggcaaattta taatgcaatt aaagatggag |
| 6181 | gagggaaaaa cttagaaaac tatgttaatc ttcctacatc tgcttccggc ctaaccctaa |
| 6241 | ttggcccatc agggatggga aagtctacga atttccaaaa tattcttaat ctttatactc |
| 6301 | aagtaattct tcatcctgag tatagtgttt accaaatagt ttggttgaag gttgactgcc |
| 6361 | ctcatgctgg ttctttaaag ggtctgtgta cggatatttt tcttgctgtt gacaggctat |
| 6421 | taggtactaa ctactttaaa aagtttggtt cctcacgcaa ctctgaagat tatatgctgg |
| 6481 | ctcaagttgc ccaaattgcc catactcatc atttaggggt tttagttata gatgagatgc |
| 6541 | agaatctggt tacagcaaga agacgtagct cagatatgtt gaatttttta gtaaaaatgg |
| 6601 | acaatactgt aggaattcca gtaattcgag taggtactaa tgaggctttt cctattcttc |
| 6661 | aaggaaattt tagaaatgcc aggagaggga caggagaagg cagtgtaatt tgggacagaa |
| 6721 | tggtaaatga tgatgaatgg tatttcttca tggaaggaat gtgggaatac cagtggacaa |
| 6781 | aaactcctgt tcctttctcg gatgaaataa acgaggtttt ttatgacgaa agccagggaa |
| 6841 | ttattgatat tgctgtcaaa ctttacaaaa tggttcaatg gagagcgatt tcaataggtg |
| 6901 | gtaaagaaat tattactatt gagttaattc gtcaagctgc taaagacgga ctatatttag |
| 6961 | tcaagccaat gcttgatgca attaggtcag gggacaagga gtggatgatt aagtataaag |
| 7021 | atattgcgcc cctagatact acagaatatc aaaataaatg tctatctgag cttgattcca |
| 7081 | aggatttgaa agaaattcgc agattggcaa gaaagcaaca gaatcagcag aaagtatcgc |
| 7141 | cgaagctaaa ttatgtaatt cttgagcttt tgaacttgga agtagaacct gctaaagcaa |
| 7201 | aggaatgtgc agaacaagta attgtttcta gtgatgaaga tgccgaaatt ccatctttag |
| 7261 | tcaaaaaggc atatgctctt gctttgcaag gagggcaagt aaatgagagc ctgactaaga |
| 7321 | caaatactaa gaaagctaag ttaaagccca attatcaaga aaatgatatt cgacgaattg |
| 7381 | ttgaaaatgc caataaggag aaaaaatctg catacgagga tttgaaattg gcaggcataa |
| 7441 | tcaaaaatcc agttgaagat tttataaata tttagcttct caatgcttag tttcttccca |
| 7501 | actctgtatc ccgatgaact gttatatagc actttagcta gataccacat tcggagtgga |
| 7561 | aataaaagtt tcaagcaaac tgacttagag ctattcggtt acagttctca acaagtatgc |
| 7621 | aaaattactt taactaataa tttaaattat ttagtgaaaa acttaccttt acgctcccag |
| 7681 | caaaaagttg agaatttgct acaaaatcat acgttatatc ccttttacgc aactttttta |
| 7741 | atgccacagg aggcttggtt actaaaaaac tcaattagta aaaaactcag tggctccata |
| 7801 | ctagatgttg ctaaggtggc tactaattct attggtgatt caaaaaaatt tttaaaattt |
| 7861 | tgtcctgaat gtttagaaaa agatacccaa aaatatggcg aaccttactg gcatagaata |
| 7921 | catcaagtac ctggcatttt agtttgtcca actcatggta tcgttttaca ggatagttgt |
| 7981 | gtaaaagttg aatcgaaagg cgtacattat catgctgcga gcgtagataa ttgtttagta |
| 8041 | gcctgcaagg tcaaaaattt gacaaataac acagtaaaaa cactcttcac actggtgaat |
| 8101 | gatattaatt ggctaattca tagcaatttt gcctttcaag gtttggcttg gcttcgcgct |
| 8161 | cagtaccagc gttacttagt ggataaaggt ctgctacaaa cctttcctgg tggcaagttt |
| 8221 | aagtttgatg agtatggttt tgcccagctt atatttgagt tttatggtca agaatcctta |
| 8281 | gaagccatca atcccaagtt gactcaacat caaggtaaat acttttctca ctgcttgttt |
| 8341 | ggatgcgact taagcccagc aattgaccgg ataactcata tcttattgat aaaattttta |
| 8401 | gcaaattcta tagaagagtt ttttcagtaa taatctgcat ataaaaactg ttaaatacat |
| 8461 | taatataaag taataaataa ttaagaaaat tatctttata ggttggtatt tcaagtattc |
| 8521 | tcagattcgt gagtaaggta aagtaagtaa tatcatattt atttcacacc acgttgcatc |
| 8581 | tactgaaatt tggcatctaa aaatgattgt tgatggcaac ttacttaggc agattccctc |
| 8641 | agaatcacca ccaaagccaa gagagctttt aatgtctcag ttaccatata tggatttaac |
| 8701 | ttttaacctt attggtgaga cattaccttt cgaccacggc tatgagctat tttctgcgat |
| 8761 | cgcccacttt gaacccaaac ttcatacatt agacacgctg ggaattcaca ccattgcagg |
| 8821 | tattcccaag gatggtgtaa ttaatctgac ccaaaattct aggcttcgtg ttcggatacc |
| 8881 | agtaaatcag gtacatttag tttacccatt ggcgggtaag tccttgagaa ttggtaagca |
| 8941 | tacaattcgt ttgggtattc ccgacatatg tttgcttcaa cctgccgagc aattgcgatc |
| 9001 | gcgcattgtg gtaataagag gtcatgaaga acctgaaact tttttagtcg tagcgcagcg |
| 9061 | tcagctagaa cagctgggta tccaagcaac cgctagcatt cccaccaaag ccaatggcaa |
| 9121 | accaatacgt aaaactatca aaattaagcg atttacagtc gtgggttttg gattagaagt |
| 9181 | tacaaattta agcaatgaag actcactgac gttgcaaata catggtgtag gaggaaagca |
| 9241 | aaagatgggt tgtggactct tcatgcccat tcaagaaagg caatagcaat tactcgaaag |
| 9301 | cagataatga agacagcagc taaccccaaa attcagctta atctcggcga tcctagtatg |
| 9361 | accttattgc atcgtgctgg ggtggcagga ctttggatga cgctcaaaaa actggaaaaa |
| 9421 | atctatccaa ctgctgctca acgtattggt aattttactt ggttgctaac ccctagtaat |
| 9481 | atcagtcttt attgggaagg acaggatttc acagttttag actggttgct gaagcattca |
| 9541 | tttcagatca gtgatgaggg tttaatttcc ctaacagggt tagattctca aaacatggat |
| 9601 | attcaaacgc aaataattat ccaccaaggt atcacagcaa cttttataca acataataaa |
| 9661 | ttttttaagt ctgcgggtca acaatctaag caactaatta ttgatggtat tcaactcagg |
| 9721 | gttgattata agaaagctgc ttcctatgct catcaacatt ttgctaaaca tttatgtgac |
| 9781 | cagtacggac aattgctaca agagccaatt ggtataaggg ggtggcttta tccaggagca |
| 9841 | gtagttcgcc attatgcttt taaagaacaa accgcatttg aagaaaaagc agaatatgct |
| 9901 | ctagcgttgt tgttcgctcc tgtggcttgt caatattttg tgctgcgatc gcacaccgaa |
| 9961 | ccatcacata caaactatgt tctggttgtt cctgaagtaa ttgatttaga actttatgcc |
| 10021 | cagtatttgt ggtatttgag taaattagat tataaacatt ttcacgtatc tagcttagga |
| 10081 | gatgctggat taaaatttat tacttatgaa gcaaaacaac tagataattt caattttatc |
| 10141 | aaacgatgtc aagtaatctc ctttagtaca aaagcttggt cagagcggca aaaaactcgt |
| 10201 | gcagaaatag caatcataga attaaattct tcagttagtt atctctataa acttagttgt |
| 10261 | atttgttttc ctaattgcca agttgttata tataaaaatc aacatttttt gcttccaagt |
| 10321 | ttaatgaaag gaataattgc caataatcta gcaatgggtt ggccttggtg ggctaattgg |
| 10381 | atctcagtta ttaaaaataa taaatcattt aaacaattta ctgatgagta tataggaatt |
| 10441 | aataaaatga ttcaggactc agaatgggac atagaagctc aaaaattatt tattcaagct |
| 10501 | tgtcatgaag cattaaggaa aatatacgcc aagatttata gcagaatgaa agaaggtgga |
| 10561 | tatgctcaaa tagaacgaga aaatacacgc attttatctc agctaaaacg ctgcaccaat |
| 10621 | gctgacaatt tcagaaaatt tattgcagag ttttggggga gggcagggca aatgacaatt |
| 10681 | ctggaggaac attgggtgga attactacca ttgactacag ggataggcaa ttggaaagta |
| 10741 | gccagggact taacttttat tgcaatggct agctatccaa agaataaaat aacagaggta |
| 10801 | taggtaaaat catcacaact atttgagcta aatcgtgagt aaacgttaga ttttttgatt |
| 10861 | tgattaagca caaaatatta attgtttagg taattgttga atgtttcatc tgttcggtaa |
| 10921 | cattttgact acttatggaa ccgccgctaa taatcgtggt gaaaatgaag gtaacacaac |
| 10981 | tactttacaa aaattaattt ggaaaggtga agttcactct accgtttcat ctgaggcaat |
| 11041 | tcgttgggca ttgcgatatc attggcaaaa tgctggctat cctgtgaata gacgttggga |
| 11101 | tgagaatgct caaccagtag cggatcacat ctgggaagat cctaattttg atgatattcg |
| 11161 | cttcattgat gatgatgtgt tgggatttat gcaagcggaa gcggcaaaag ttgaagcagc |
| 11221 | agatgaatcc gaagttgaaa tccagaactc aactcaagat gagaaccaaa caacctctca |
| 11281 | aggtgaggag aaaaaaacct cacaacgtaa gcgcaagcaa agacctaaag ggagaataag |
| 11341 | tgctagacga ggtgtattag aagtcactcg tgcagtatca acaattcctt atgctggtga |
| 11401 | tattactttc aattctatga gtggaaagaa aggacgaaca tctctttatg ctacagaagt |
| 11461 | tcacgctact cggtatcaat atggctttgc attaacaccc aatagactca aagataaatc |
| 11521 | tcgcattcat gctgtcttag atggcttaat ttcgattgga gacgtggcag gaaatcacgc |
| 11581 | tcgtttcttt tatgattttt ctcccgatag cataatatta cggtggacac atgatttctc |
| 11641 | acctcgctta ctttactgct ttgaagaaga tgaacttaga aatatatcaa cacccgattt |
| 11701 | agtgcgacgg gtagaagctg gagatataga ccctaaagaa ttatggatag gaggggcaat |
| 11761 | tgctagcact ctagaaaatt taggggccaa tctttttccc ggagtgaagg caacagttga |
| 11821 | agcattaaaa caggtgatta ttgaagattt gcagttgtcg ttaaggagtt catgatatga |
| 11881 | cgacaattgt tgtaaaagta gaagttccca tagcttgttt tcgccagtct catgctaggg |
| 11941 | aatacgctga aacctaccct gtaccaccgc catcgacagt atatggaatg ttactatctc |
| 12001 | tggttggaga agttgataga tacaaacact gcggcgttaa gctggcgatc gcacttctat |
| 12061 | ctaaaccaga aaagtcaact gttattcgta cctgccaccg atttaaaacg aagaatatcc |
| 12121 | atgaatctaa aaataataaa cctgattacc aggatttact aactaatatt gagtttgtag |
| 12181 | tttgggtaga tgctggggca gataaagcca aaccaaattt agcgcaacgt ttagaagagg |
| 12241 | catttactaa cccagcttct attaataggt ttggtggatt atgtttaggt gaaagtcggg |
| 12301 | atttagtgaa tgatgttagc ctgttacctg aaaattacta tgcagaatca atgcgatggt |
| 12361 | taattcgaga tgattatggg ttactaacct taccttattg ggtagatcat gtaggttcac |
| 12421 | ggggaacgcg atggctgtgt tacgaaattc aggaatttca agtatttcaa ccacccgaat |
| 12481 | tatcttggac ttcaattcaa agtgattaga gggcattatt tacttttgca agcatctttg |
| 12541 | cttgggcaat ttaaatacca caataaataa tcttgtcttc ttgtttcaca agcattttag |
| 12601 | ggatttcata tactttcaat gtgcttgcaa tttcctggaa caccttcaaa acaagggtta |
| 12661 | gagctttagc aactttgctt ttgctatgaa tagctattaa tttttgcaac ttgactaatc |
| 12721 | ctaattggaa ggtgcttgta aaattagtac agagctttgg ctagtcaaca ggtttgatga |
| 12781 | tcgccgtgct ttaacattag atgtcgttag gcgttgagca gtgattaacc tgaaggcttg |
| 12841 | ctccgggatg ttgtaggtgc tttaacatta gatgtcgtta ggcgttgagc agcaccggta |
| 12901 | ccgcgagtag tcgcaccaat attcatatgt gctttaacat tagatgtcgt taggcgttga |
| 12961 | gcagttgaat gcgaagttat cggcagatgt tttgttgttg tgctttaaca ttagatgtcg |
| 13021 | ttagacgttg agcagtgacc attggtgtat ccatcaatcc tgactgctgc gtgctttaac |
| 13081 | attagatgtc gttaggcgtt atctactttt gatacaaata cttttgaaat acatacaaaa |
| 13141 | ctagctctgc caaatatctc cttttcaaga catttttgcg attatttttt cccactgctc |
| 13201 | aatctggttc tgtagttgta attgccgatc gcccaaatct tgactatcat caacagacaa |
| 13261 | atcctgaccg ctcaatcgct cacgatagag agcaatcagt tcggttaggg cttttttatt |
| 13321 | gcttgctgat cctcctgggt cagagaacta acattgactg gaatcaactt tctgccaatt |
| 13381 | ctctctacaa actcagaccg acttaaatcc atactgtcgg cgatcgcatc taatccatca |
| 13441 | atcccggtag gagttagggc taggttaact cgtttcttca cctcatcgta cagttctggt |
| 13501 | acgtcttttt gacctttttg ccctttctta cccattgccg tctagttttt gctttcctcc |
| 13561 | caatcatagt aaacacattt agcacttaag ttaaacatta aaattttttc caatacttta |
| 13621 | ggtattgtgt aatccctaat ttagttagat aatacaggca ttgtgtaatg cctaagcagg |
| 13681 | catctcgtaa ggatatgagt aactaattgc accgtttgat tcctcaacat ctcaacggct |
| 13741 | taattttcat gggcaagcat taattgcttc gtggccaatt cagattaatc aaaatctgcc |
| 13801 | agatgattta ggcatttttc aaaactatat ggttagtttc ttccctaaac cttatccaga |
| 13861 | tgaaatttta tatagcgtca ttgctcgtta tcacattaga agtggcaata cgagtcccaa |
| 13921 | aattacactt caagagttat tcaattcaca aactactgtc gcaacagtag atttaccatc |
| 13981 | gaacctgaat aatttaattc aaaacctaca atttatctca aattaccaag ttgaagattt |
| 14041 | gatatataaa catactctct atcctttata tagccctttt ttacctgcaa acaggtctag |
| 14101 | tcaggtactg gaatctatga aaggagatta tgggggagat attcatacaa gagccggaat |
| 14161 | tatggcaagc tcaattacta tgccaaaata ttttcggttt tgtccaactt gctgggaaga |
| 14221 | agacttaaaa aattatggcg aaccttactg gcatcgcatc catcaaatac caggggtttt |
| 14281 | agtttgtcca tttcatgatg aagttttgca agatagccta gctcctttac acggttttaa |
| 14341 | taagcatgaa tattatgcag ctagtacaga caaatgttct attactaaag aaccaagtat |
| 14401 | ttttagccct gacacgttga aaaaattact ggtattagct aaagattttt ggtggttact |
| 14461 | caatagtgaa ataccttgcc aagaaccaca gtggtttcgc aaacagtaca ccaatctgat |
| 14521 | gattgagcag ggtcttgcaa ctgcaactgg acgaatttac cagaaaaaac tactagataa |
| 14581 | ttttttgttt ttttatggtc gagaaatgct cagtgccgtt aattcaatag tcactcataa |
| 14641 | tgattcaagc aactgggtta ctagcattgt caggaaacat aggaaatctt tccatcctct |
| 14701 | tagacatctg ctgatgatgc gttttttgaa aacatcagta gaggaatttt tcaagcaaga |
| 14761 | taataaatac aagccttttg gtgaaccccc gtggttgtgt ctcaatgctg ccgcagagca |
| 14821 | ttacctacaa ccagtcatta ccaatttagt cattacttta tgctgcgata caaaaaagcc |
| 14881 | tgttggtact ttttcttgta cttgtggaat ggtttattgc cggactgggc ctgatgaaac |
| 14941 | ggatgatgac aaacttcgca ttggtaaagt aaaggcattc ggtcaactat gggaacaaaa |
| 15001 | gctgaaagaa tttgttgaag ttgaaaagtt gggtttgcgg gagacagcta gaagattaaa |
| 15061 | tgttgatccc ggaactatca aacgctatgt atctctgcta aaactgacca cttattggca |
| 15121 | gactagaaag gaaaatggct ctgtagactt acaggaagta gcagaaatac accccaattc |
| 15181 | tgcaactgaa ctcaaattga aacacaggca agattggatg gctttacaag cacagaatcc |
| 15241 | tgacgcttca aagacaactc tgagaaaact agcaccagcc acctatattt ggctatacag |
| 15301 | aaatgataga gaatggctca atcaaaactc acctgcatta caagttgctg ttccttccgt |
| 15361 | tgtgagagtt gattggcagg aacgagatcg gcaaattctc gcccaggctc aagacgcagt |
| 15421 | gcgatcgcta attaacgccg atacgccaga aagaatttct attagtaggg taggaaaaac |
| 15481 | tatcggctta ctcgccttac tcgaaaaaca cctcgaccaa atgcctttaa caaaggctta |
| 15541 | cttagaatca gtagttgaaa ctgtagaaga ttttcagatg cgccggatca agtgggcaat |
| 15601 | caagttgctt gatgaatgtg gagaagaaat tatgcgctgg aaggtagtca gagttgctgc |
| 15661 | tttgcgggag gattgctctg aaagagtcaa cgcatttctg gaaaatgaac ttgacaaagc |
| 15721 | gtacaagaaa gagaggtgat tccattcacc tctccaagtg atgagataaa tgctttgtgt |
| 15781 | gttatgctgc gtcggctacc agtaagccat tcacagcgac gatgatgatt tgcgctgcca |
| 15841 | aaggagtgtc acaacgtagc ttatcgtcac tattacaggg ttgtgctatc cattgaccct |
| 15901 | ccagattttg gtagaaagtg ccgagaagtt gggaaccatt ccaaacacgg taaagttcac |
| 15961 | caaatacctc tgataccgaa tcaatctcaa tttccggtgc tatttcctca cctagctgct |
| 16021 | caatgtactc ctcaaactct aattgagcca aggcttggtt atcaaattca tcctgtacaa |
| 16081 | tcatcatgag tcacctcgtt aacggggttg aacaaaaggc gatggcgtaa aacgcccgcc |
| 16141 | taaaaggcga tcgcatttct cttgccggag ggcgatcgcc tttttgattt cagattttat |
| 16201 | tttagctact atagtggcta tatacaatat atgtatggaa aactttattc agtcacttcc |
| 16261 | actgtgctaa gaaaatcctt taaagaaatg cctaaagcca agcaaatagc ctctaacacc |
| 16321 | ttgatagaga cactttcaga ctcgccgtac tctagttttt tcaggttttg gtgagagcat |
| 16381 | tccccaccct tagcagaaat ttcctctgcc aatttctgcc gggatttttt accacgcata |
| 16441 | cgctttaaaa ctgctgctct atctttattc cagctaacgg ccacaacgtc tgaaacataa |
| 16501 | atatacattt tagtttctct attcctctca ttgttcatat aatagctaca gcagtaaaca |
| 16561 | aaagttatta atttcggcta ctacagttta caaattatgg gttagaggta agcgatcgca |
| 16621 | gttaatcaag ttttcctgtc agtcaaaaga gagtaactgt ttttgctatg aataccactt |
| 16681 | gtgcatataa tttcaaaggt tctcccagaa caaatacatt agccatctac agcatctaga |
| 16741 | gccttgacac ccgaagaact aaaacagctt gaagacaccc tatggcagag tgcagatacc |
| 16801 | ctgcgggcta actctgacct cacatcgaat gaatattcga cccctgtaat gggtttaata |
| 16861 | tttctcaagt ttgctgataa taaatatcgt cagtatgaag cggagatttt acaggcttat |
| 16921 | gaagcattaa aagggactcg tagagagaag aatatagaag aaatagctat tgagaaatgt |
| 16981 | ggtttttatt taccaaatca cgcgcgttat gactatttat taaacttacc agaagaggaa |
| 17041 | gatatagcta aaaaaattaa agaggcgatg gagtccatag aaaattataa gccagaactc |
| 17101 | aaagacacat tgcctaaaga tgaatatttc cgcctcactc gtacagacaa aggtattcct |
| 17161 | aaacaactat taaaaaattt ttctaatatt cctcaaaacg ccactgggga tatatttgga |
| 17221 | cgaatttatg agtatttttt aggtaatttt gcactttcag aaggacaagg tggaggtgag |
| 17281 | ttttttacac cccgttctgt ggtgcggcta atggtagaaa ttattgaacc attaaaaggt |
| 17341 | agagtatttg atcctgcttg tggttctggg ggaatgtttg tacagacagg acaatttatt |
| 17401 | caagaacagc aagaaaatca aaatcagggt gtaattaatg atttatatgt ttgtgggcaa |
| 17461 | gaaaagagac tggaaactgt aaaactagca aaaatgaaca ttacagttaa tgggttaagg |
| 17521 | ggagaaatca aacaagcaaa tacgtattat gaagacttgt ttaatagtct tggcgatttt |
| 17581 | gattatgttc tggctaaccc accctttaat gtggatgatg taaatctgac acaggtggag |
| 17641 | aaagacccgc gatttaatac ctatggtatc ccccgcaata aaacaaaagt taaaaagcag |
| 17701 | gatgagggta aacaaacagt acctaatgct aattatctgt ggattaattt gtttgctaca |
| 17761 | tccctaaaac ctaaagctag agcagcgtta gtaatggcta actcggcttc tgatgcgcgt |
| 17821 | cacgcggaag cagatattcg ccaaaagctg attgaagaga atttaattta tgggatgctt |
| 17881 | gctctgccct caaatatgtt ttacacggtg acgctacctg cgacattgtg gttttttgat |
| 17941 | aagggcaaga ccgatgataa gattttgttt attgatgccc gaaatatttt tacccaggtt |
| 18001 | gacaggtcac atcgagaatt ttcagcagaa caaatcagta atctggcggt tattagttat |
| 18061 | ttgcgtcagg gtaagcgaca tcggtttatt aagttgatta atcgctattt tcagcaagga |
| 18121 | atggcgaagt taaaagaaaa tcaatcacag gtaaagcctg tttctcagca attaatggct |
| 18181 | gtgttgaatg atggtgcaga tgatgtcaaa gctagggaag ttgctgttaa tttcctcaat |
| 18241 | ttgtgggatg aggtaacaga tttagaaatt cagtatcaag gttatttaga taaatataat |
| 18301 | tatcctggaa taaatattga tacccaaaat gaggcacagc aaaaactaag tgccgcattt |
| 18361 | aaaccgtttt ttgataattt ccatgttgag ctaaaacagc ttgataagac tgtgcgcgct |
| 18421 | cgtgaagcag agctaaagga gaaagcaaag gaagcagagg cgaagaaacg ggggcagatt |
| 18481 | gataaacaaa ttaaggaact gaaaagcact ttggaaacgc tgcatgagga ggtaaaatca |
| 18541 | gcagaattat tttttactca tattcgctgg ttacaggaac gttttcctaa tgcaaaatat |
| 18601 | gaggatgtga cagggttgtg taagttggca actttggagg aagtgaaaga acaggattat |
| 18661 | tcgttaaatc ctgggcggta tgtgggggta gtgattgagg aagatgggaa aacggaagag |
| 18721 | gagtttattg aggagatttt aggtttaaat gaagagttgg aaaaattaaa tcaagatgtg |
| 18781 | catttgttag aggggattat tgctcataat ttacgtgaga tagcggggga tttatgagtg |
| 18841 | attatttaaa tcaatttcag aaattagatg aagtggggac tattgcaaga ggtaaatcaa |
| 18901 | aacatcgccc aagaaatgag ccatcgttat atggtggaaa atatccattt attcaaactg |
| 18961 | gagatgtaaa acatagcaat ctttacataa ctgagtattc ccaaacatat aatgaaaaag |
| 19021 | ggctatctca aagtaaatta tggcaagcag gtacattatg tataacaatt gcagctaata |
| 19081 | tagctgatac tgcgatatta aaatttcctg cgtgctttcc tgatagtatt atcggattta |
| 19141 | taccctacaa aaataaagct gatgttcgat ttatcaaata ttgtttagat acatataaaa |
| 19201 | ttcaaattca ggcaatttct caaggcacaa cacaagacaa tttgagcgta gaaaaacttt |
| 19261 | tatcgcttaa atttagaatt ccattaattt ccattcagaa aaagattggg gcgatacttt |
| 19321 | cagcttatga tgatttgatt gaaaaaaata atcgccgtat tgccatatta gaaaaaatgg |
| 19381 | ctgaggaaat ctaccgtgag tggtttgtcc gtctgcgctt tccgggacat gaacaggtta |
| 19441 | agtttaataa aggcatcccg gaggggtggg agagaaaaag attcgatgaa ttttgtctac |
| 19501 | tccagcgtgg gtatgatcta ccagatgcta aagtaattcc tggccaatat cctgttatcg |
| 19561 | cttcaacttc aattaaaacg taccataatc aatttaaagt taaccctcca gtaattacaa |
| 19621 | ctggtcgctc aggctctctt ggcacagttt tattcatcaa tagtcaagcc tggcctttaa |
| 19681 | acacgacatt atttttaaaa aacttttatg gtaattcacc atacttaatt tactacacac |
| 19741 | taaaattctt aaaactggaa aattttaatt ctggtgctgg tgtacccaca ctaaacagaa |
| 19801 | atcatttggg tggattgtat atgtctgttc ctcctaaaaa cttgcaaaat aattttactg |
| 19861 | acaaaatagc aattattttt aaacagaaag agcttttagt taggttaaat aataaacttg |
| 19921 | ttgaaacacg cgatcgcctt ctcccccgcc tcatctcagg taaactttcc gtcgaaaact |
| 19981 | tagacatcca attcccaccg agtatggctg acacaccacc ataaaagtaa tactcagtgt |
| 20041 | ttatcctata aagtcagggc agatattgac aaatttaagc agcaatgggc taaaatttgt |
| 20101 | acattgatgg actaataata tcaaaaacag cctatagccg tgtagtttgc ctaaactatg |
| 20161 | cctaacttta tctctgaaga tgacatcgaa caagcggtac tccgcaaatt agagcagcag |
| 20221 | cacggtttta aattactcaa ctgctacacc ccaaaccctg acgacctcaa cgacagttca |
| 20281 | aaccgcacag ataaacgaga tgtcattttt agcgatcgcc tgaaagctgc ggctatacgc |
| 20341 | ctcaacccca ccttaccaga aacagccata gatgaagctt taaaaatcct caccagtcaa |
| 20401 | cgtcagtcta tgtctcccat cgctgcaaat cgggaaatag acgggctgat tcgtgacggt |
| 20461 | atccccatac aatatgaaaa tgcccaaggt agaaccgaac ccatcaaagt acgagtaata |
| 20521 | gattttaacg atttcaggac agatttcaat aatgaatatt tagccgtttc ccaactgtgg |
| 20581 | attaaaggag aaagaaatta tcgccgtcct gacatcatcc tctacatcaa cggtttaccc |
| 20641 | ctggttttca ttgaactgaa aaattccaac gtcaaattac aaacagcctt tgacgataac |
| 20701 | ctcaccaact acaaaaaaga tattcctcaa ctcttccttg ccaatgcttt ctgcattctt |
| 20761 | tccaatgcac tagaaaccaa aattggcagt tttaccgcag actgggaaaa cttctttaac |
| 20821 | tggttacggg tagaagatga aaaggaaaaa ataaatcgtc agcagattca ggaatctggt |
| 20881 | actagcctag aacgagccat agatggtctt tgtcaccctg ccaaactcct agactacatc |
| 20941 | gagaatttta ttatttacag aagtggaact cagaaaattg tcgcccaaaa tcaccaattc |
| 21001 | attggtgtga atcgcgcaat tgatgccttt atccatcggg aagaaaaaca ggggaaatta |
| 21061 | ggcgttttct ggcacactca aggctcagga aagagctttt ctatgatttt ttatgtccga |
| 21121 | aagatttcgc gcaagctcaa aggcgatttt acctttgtgg tggtaacaga ccgggacgat |
| 21181 | ttagacgggc agatttaccg caatttctta aacaccgaaa ccgtccaaga gaaagaagct |
| 21241 | gctcaaccca aaaacagtaa acaactgcgg gattttcttt ctttcaacaa gcggatagtt |
| 21301 | tttaccttga ttcaaaaatt ccgctacaaa aagggcaaga agtatccaat attgagtgaa |
| 21361 | cgtaacgata tcatcgtcat tgttgatgaa gcacaccgca cacagtataa atccttagcg |
| 21421 | gaaaatatgc gggcaggttt acccaatgcc aactacctcg cgtttacagg tacacctctg |
| 21481 | ttaggtaaag aacgcaaaac taatgaatgg tttggggatt atgtcagcga atacaacttt |
| 21541 | tctcagtcaa tagatgatgg tgctactgtc cccctgtttt accagaagcg agtaccagag |
| 21601 | gtactgattc aaaacgagaa tttaagcgag gaattttacc aaatactcga agatgaaaac |
| 21661 | cttgatgata cgcaacaagc caagctagaa aggaaatttg cccgtgaaat tgaggtcatt |
| 21721 | aaacgtgacg atcgcttaga aacagtagcc aaagatatag tttaccactt tccccgtcgt |
| 21781 | ggttatctag gtaaaggttt ggtagtttcc gtagataaat tcactgctgt gaagatgtac |
| 21841 | gacaaagtac agtatcactg gaaagcagaa attaaaaatt tagtgggacg aattaggaaa |
| 21901 | tctaataatg aaatcgagaa agtgcgcctc aaaaagattc tcgattttat gcgcgaaaca |
| 21961 | gaaatggctg tagtgattag tttagaagga agcgagagcg aagagaaaaa atttgctcaa |
| 22021 | caaggactcg atatcaaacc ccaccgaaaa agaatgaata cccttgatgc tcaaggtcat |
| 22081 | gatatcgagt atcaattcaa agattcagaa aatcccctgc gcttggtgtt tgtctgtgcc |
| 22141 | atgtggttga caggttttga cctacctacc ttgtctactc tctacctaga caagcccatg |
| 22201 | aaagaccaca cgctgatgca aactatagcc cgtgcaaatc gcgttagtag ttatcagatt |
| 22261 | aataatgtca ctaaaaccaa cggcgagatt attgattact acaatgtctt ccgtgatatg |
| 22321 | aaaaaagccc tagcagctta cgctttgggc aatgatgaaa actccctagt tccagaaaaa |
| 22381 | tctgacctgt ttaaattact tgatgatgcg atcgcgcaag gactaaactt ttgtcatgaa |
| 22441 | cgaggcatta atctagaaac tatccttgcc agtcaagata ccttcaagaa cttagagcag |
| 22501 | tttaaccagt ttgccgacac cctactgcaa aaagatgaat ggcgtaaagc ctttgtggtt |
| 22561 | tatgacaata ccattacctc actctacgaa gcctgtaagc cagagatatt aaaccaacca |
| 22621 | cgccccctgg tgtcagtttt tcaatactta cgaggagtca ttgatagtat tatcgagcgc |
| 22681 | aaagatattg gtaacgtcag tttaaaaatt gccgagttat tagatgaaag tatagtcaca |
| 22741 | gatgatcaag gagtgaccac acaagagtat agtgcgaaat accaaattat ccagaaaggt |
| 22801 | gaagtttggg atttaagtaa aatcaactgt gagcagctaa aagccgaatt taaacacaaa |
| 22861 | ccctataaaa atattgcaat taccgattta aggagcttca tcgaagacaa gctcaaccgc |
| 22921 | atgattcagc aaaatactac tcgtacagac tttgcccaaa gactacaagc aatcattgac |
| 22981 | aaatataacg ctggtggttc atcaactgag aactactacg aagccctggt aaactttgcc |
| 23041 | gaaaacctga aacaagaagc agaacggcat attcgggaag gcttgacaga agacgaactg |
| 23101 | gaactatttg atttactcaa gaaagacaaa atgactgcgg atgaaaccca gaaagtcaaa |
| 23161 | ttagctgcca agtccctttt acagcgactc acagcagagc agccaaaagt tttagtgcaa |
| 23221 | gattggtata aagatatgca gagtcaacgc agggtgaaat ccgtagtgga agcagtctta |
| 23281 | gatcaaaatc tgcctgaaag ctatgatcga gcgttattta agtttaaatg cgacacagta |
| 23341 | tttgatttga tctatgacca ggccagtaaa ggtgcaaagt gggcagtctg agtaatggaa |
| 23401 | actatgtcta tctatgaacg cgatcgcatg aagacgaaaa tcagatttat gagatttgta |
| 23461 | cggctggcat aatttatcac tgctttcgac ttttcaagta gctctactta agcgataatt |
| 23521 | agaacttgaa gacacatgat gactataaca aaatgggcca gcatttatac gctttacttg |
| 23581 | ttggtatcga caactatcct gaccccaatc atcgccttca agggtgtgtg aacgacataa |
| 23641 | cagtaataca agagtatctt aacgaacggg tcgatcagca gcagtatgaa ctgcattcat |
| 23701 | tgatacttaa aaatagtgaa gccactcgtc aggcggttat taatggcttc cgcaaccatc |
| 23761 | tttgtcaagc tcaacaaaat gatatagttc tgttttacta cagcggtcat ggttctcagg |
| 23821 | agttagcacc gaaagaattt tggtatttag aaccagacca tctggatgaa actttagtat |
| 23881 | gttatgacag tcgtactgag aacggctggg atttggctga taaagaattg gcactactga |
| 23941 | ttggggaagt cgcagaaaac aacccccata tcacaattat tttagattgc tgtcactctg |
| 24001 | gttctggtac taaagatccc atgcagacag ctaaggaacg tcgcttcccc acagacaagc |
| 24061 | gatcacgtcc attagatagc tttatctttt ccctcgacga tttagagaga ctttcagata |
| 24121 | cccacaaccc cgaaaaacac ccgactgggt ggaaaatccc cagaggtcgt catgtacttt |
| 24181 | tagcagcttg tcgagaccac gaaacggcaa aggaatacta tggtggtggc caacaccggg |
| 24241 | gtagtttctc ctatttctta ctggataccc tttctaaaac taacggaaaa ctaacttatc |
| 24301 | gagatttatt tgcacgcacc aacgctattg tccgcagtga aattacagac cagtctcctc |
| 24361 | agttggaagt aaatgatcct gaagatgaca ataaattttt tctcgatcgc gtgatcgctg |
| 24421 | aacatgaccc ctattttatc gttcagaatc acagaaccta cggatgggta attgatggtg |
| 24481 | gtgcagttca cggagttcaa gcacctgaac atggagaaac tactttacta gcattgtttc |
| 24541 | cctttgatgc caatattgat gatctacgtg acccatcgaa atctgtgggt atagcaaaag |
| 24601 | ttactcaggt actaccaggt aagagcaaaa tagatattga aggtatacaa aacttagctg |
| 24661 | ctgataacac ttttaaagca gttgtgacga gtctacctct accaccttta ggagtgtact |
| 24721 | ttgagggaga tgaggatggt gtcaccatag caagggaaaa gctgagaaca gtgggtaatg |
| 24781 | gtaagtctcc atcaccatat ctccgcgaag agaaagaagc tagtcaagct gagtttcgtc |
| 24841 | tgttgtgtca caacaaccaa tatgtaatag ccaaaccaac agatagccga cctcttgtag |
| 24901 | aacagattga tggttacaca ccaaaaaatg cagaaatagc aattaaacgc ctagaacata |
| 24961 | ttgcccgttg gacaacaatt gctaaacttt caaatactgc ggcaactcgg attaaagctg |
| 25021 | gtgatgtgaa gatggaactt atctttgctg atgaggattt accacaatca aaccagatgc |
| 25081 | gtttgccata caaatataat aatggaacat ggcaacgacc agaatttaga cttaaactca |
| 25141 | ccaatacgac gaaaaagcca cttttctgtg ctttagttaa cctgtcagat agtttctcca |
| 25201 | ttagtacccc gttttttgaa gcaggtagca ttagactgga agcagaacag gaagcttatg |
| 25261 | ctttagatgg agaagaattg caactagaag tcccagacga gtattgggaa cagggtatca |
| 25321 | cggagtacaa agacattatt aaattgattg ttagtaatga tgagtttgat ccaagattgt |
| 25381 | taaatcaaag caaacttgat gctcctcggc ttaaagcaat agataggaat tatgcaaatc |
| 25441 | aaagtagtct tgatcggttg atggatcgta ctcaaaatcg agaaattaga gcgaaaagtg |
| 25501 | acgctgttag atttgatgat tggtacgctg aagaaatcac aattactaca gtccgtcccc |
| 25561 | tgcaatctac accagtttct cagcaacaag aacaacaatt aggcgcaggg gtaagattac |
| 25621 | aaccacatcc taatttagtg gcaaatgtta gcctcacaac tataccagaa gtgagccgag |
| 25681 | atttgggtaa caacattgta cctcctatct tgagagaaaa ttccgaagtt attcaaccgt |
| 25741 | tccaatttac tgccagtcgt gctactgacc ctggattaag tgtattggaa ttaaacgata |
| 25801 | ttcaagatta caaagttgtt actcctaatg cacctctaaa attatttgtt gatgcaccac |
| 25861 | tagcagataa tgagtatatt cttcctatcg gctatgacgg agaatttttc ttgccgttag |
| 25921 | gtcgtggtca taaaacttta catggtcaaa cagaaattga gatagaacaa ttaccagccc |
| 25981 | cagttagtca aggagagaaa agcctacaag gttctattcg catctttttt caaaaaatta |
| 26041 | tcaaccaaaa attaggaaaa aaatataact atcctctact cagagtagta gaagttaatg |
| 26101 | ataatcaaca agttaattat ctaccagatg aggtaactat taaccgagtt aaggaagcaa |
| 26161 | aaagaattgc tgtttatatt catggaatta ttggcgatac tgaaagttta gtggcaacca |
| 26221 | tcaaacagcc tatattacaa ccagacagac aaacagtttc tatcagcgaa ctttatgatc |
| 26281 | taattcttac ctttgactat gaaaatctca acacttccat tgaaaataat gctcaatatt |
| 26341 | taaaaaagcg attgagagat gtaggtctgg gagaaaatca cggcaaagaa ttacatatta |
| 26401 | ttgctcattc aatgggggga ttggtatctc gttggttcat tgaaagagaa ggaggaaata |
| 26461 | aagttgtcca acacttgatt atgttgggta caccgaatgc aggttctcct tggccagttg |
| 26521 | tagaagattg ggtaaaactc actttaggta ttgcacttaa tggtcttgct atcattgctt |
| 26581 | ggactgcaac tgtggtagct ggactaatgg gtgctttaga aagaaatatt cgggtagcat |
| 26641 | tagcccaaat gaacccagct tctgattttc tcaattctct agcagccagt gatgaccctg |
| 26701 | gtattcctta ctcaattatt gctggtaata cttctatgat tacatctgtt gttccagcag |
| 26761 | cattagagca acaaccagag aaaaattcta gtgtgctaga acgactcaag caaagtttat |
| 26821 | ttaatagagt tgtagcttta ccttttggtg gtgaaccgaa tgatattgct gtgacggtgg |
| 26881 | aaagtattaa tagcattcct caagggcgat cgcatcctcc ccatattcaa gaagtacctt |
| 26941 | gcgatcactt gagttacttc ctgaatcaac ctgatgaagt tgggttacaa gctttgattg |
| 27001 | cagctattac gaatcaagaa tattcttatg gagagttaca aaaaccttat aaagatcaag |
| 27061 | aattacaggg actttatgaa ggattacaag atttatggga aaaacaagaa attcatactt |
| 27121 | gttctatttg ctcgaatttt atcacagtta atcaaaaatt tgaaggtgct agtaatgttg |
| 27181 | agtctttcat tgcgaaaaac agaaaaaaag tggaaaatat aattgttgaa attaatagca |
| 27241 | catattcggc aaaagaaata aaagataact tacatcaaat tgcaattata gggttctaca |
| 27301 | gaagtaacag tttccgttcg ttacctgcaa ttatttccca aaaagataat tttttacaaa |
| 27361 | tcgagcttga agacattttt agctttttag atcctaaaga aaatcaacaa actgaaaata |
| 27421 | ttccatcttt ttcaacagag ataattttgt ttgccgttta ctatcagaag gagaataaac |
| 27481 | atggatacta agaaagtaga agaggtaagg aagacaggat gtactcttat tgcttttaaa |
| 27541 | gatggtaagt atgaaagagc agcatctgaa gattttaaaa cagaagctga catagttatt |
| 27601 | ttaattcatg gctttacgag tcatggagaa tacatgggtc aggaattagt accttatctt |
| 27661 | tatagtgaaa attatcaaat cttcttgttt aattatgatt cttacaaagg aatattatct |
| 27721 | gctgtaagga gtttacaaga attactggaa acgtatgata atctaactaa tcaaaaaata |
| 27781 | cgctccaaga aaatattcct tattgggcat agtatgggtg gtcttgtagc tcgtcaattt |
| 27841 | actatagatg atgcaaagaa ggacaattca gataggttta taaaaggagt gatcatgttg |
| 27901 | gctactccta ataatggtgt attacaaaat aaattaagta acgaaaattg gagaaatttt |
| 27961 | gttaagtatt taatatctgt ttcagaagaa ataggcggag tattcccaca agccagaaca |
| 28021 | ttggagtgcg tcgcggtaaa agaacttgct aaaattgatg agtataatat tattgataaa |
| 28081 | ttgaattacg aatgggaaaa gatgagttgc aatttgccac caagtttatc tatatctggt |
| 28141 | ggacaaaatt atcttgaatt cactaataaa ttaattgaag gaataattaa tcgtggaatt |
| 28201 | cagcaattaa tatctgattc taatacccgc tgccctaatg atggaattgt gcttgaatgc |
| 28261 | agtgttgata tgaactcatg tattagctat ccccaacaaa atcaacaata tatacacctg |
| 28321 | aataattatc atgaatattc aaataccaat cacaatacaa ttcatatcca acaaatagta |
| 28381 | tcaattaaaa ttgtagaatg gttacaaaca atcagcaaaa aaaatacgag tttactaaat |
| 28441 | aacgcataat aataaaattg attgtagtgc ttttgaattt ttagagttaa cataacaatt |
| 28501 | atgtctaaac ataaatttaa ccttaatttt gctattatta tcggtattaa taattatcaa |
| 28561 | agcgggatca aagagctaga aacggcagtt ccagatgcta ttaaacttgc tgaaattcta |
| 28621 | caaaagcagc atgatgctct aaaaccacaa tatcaagcac aaaataaata tgaagtgcaa |
| 28681 | ttatttttaa atcaacgcgc tggtcttaaa caactgaatc aattaattgc agattttaaa |
| 28741 | caaggtcaaa taccattaga cagaggtaaa gttacagtta cagaaaatga tcgcctcctt |
| 28801 | ttttactttg ctgggcatgg aattgcttta gatgctctag aaaatcaaga aggcccagtg |
| 28861 | ggttacttaa ttcctcaaga tgctatatca ggggatagca gtacctactt gccaatgcag |
| 28921 | gaattgcatg atgctttaaa ctcacttcct tgtaggcata tgttagcaat tcttgattgc |
| 28981 | tgctttgcag gagtatttcg ttgggcaagt ctaaaaagag aaattgtgcc taaagtaaaa |
| 29041 | gtatataagg aacgctacga ccgattcatt agcgatcgcg cttggcaagt aattacttct |
| 29101 | gctgctgatg accaaacagc tttagattct ttaggttcac ggggtatggt cacagagggg |
| 29161 | aacgaagttc attctccctt tgccaaagct ttgtttgatg gtttatttgg gaagggtgca |
| 29221 | gacctaaata aagatggtat catcaccgca actgagcttt acttgtattt aagagatcaa |
| 29281 | gtcgaaatcc ttacagaaaa taactacaag cggcaaaccc ctggtttgtg tccactgaaa |
| 29341 | aaacacgata aaggggaatt tatctttttg tcgtctgaat ttgaccgaaa taatttggag |
| 29401 | gatgcaccgc cactaaattt agaaaataat ccctatcgag gattagaatc ctatgatgaa |
| 29461 | aaacacagtc atttgttctt tggtagagaa gaacagatta aaaagcttta ccagaaagta |
| 29521 | gttgctaaca accagaagtt aacattagtt ttgggtgctt caggtactgg caaatccagt |
| 29581 | ttggtgaaag ccggacttat tcctaaactc aggaaagata aaacttggtt tatcctaccc |
| 29641 | ccttttcgac caggggagtc tcctttcaaa tcattgaata acgcactaga atcggttaag |
| 29701 | cagcctttga ttgctgcttc ctcaacgacg acaccttctc gtttgcttac cccagccgaa |
| 29761 | gaaagccttg caaattggtt taataatcat cctcaagcaa agctactggt ggttattgat |
| 29821 | cagtttgaag aactgataac tctgtgtaaa agtgacaaag agcgagagca atttgagcaa |
| 29881 | ctgattaaaa atgcaattac caaatatgcc gacaaaatcc atgtagttat caccttgcgg |
| 29941 | cttgactttg aagcacagct tcaaaactct gtacttgaaa agttctggaa tgatgagact |
| 30001 | cggtttgttg tcccaccaat gagccagaat gaatttcggg aagtgattga gaaacctgca |
| 30061 | ctagaaaaag ttatatattt tgaccctccc agcttggtag atgacttgat taacgaagtt |
| 30121 | gtacaaatgc cgggtgcttt acctttgctt tctttcaccc tcagtgaatt gtacttgaaa |
| 30181 | tatttgcagg agcggagaga taaccgggct ttaacgaaag aagattacga acagttagga |
| 30241 | agagtagttg gttctttaac caaacgagct aatcaggaat atgatcattt aattcaagaa |
| 30301 | aaccccgctt atgaaaacac tgtacaacag gtaatgttac gaatgatttc attgcagggt |
| 30361 | agggagtcag cacgacggca agttcctaaa tcggaactgg tgtatgccaa tgaggaagaa |
| 30421 | aataaacggg ttcaaagaat tattaaatgc ttttctgaag ctcgcttaat cgttgagggt |
| 30481 | tctaattctc aaggtgaacc ttatgtggaa ccagcacatg atgctttagt acaggggtgg |
| 30541 | gttaagttac aggagtggat taaacaagag caacagaatt tacctttaca acgacgattg |
| 30601 | acattagctg tccttgagtg gaaaactaag aaacaaagcc aagataggca agaagcaaag |
| 30661 | ttcctttgga atgccgatcc ttacttagat gttttaaaca aaatactcaa ttccagtgac |
| 30721 | aattggctta accaagtaga aactgagttt gttaagtgta gcaccaacct cagagaaaag |
| 30781 | aatcgtcgca ttagaatggg cttggttgct accgcattct cttttatctc catattttct |
| 30841 | tttatacaat ggaaacaaaa tcaagaagct cagagtatca acttgagtgt atcttcaaaa |
| 30901 | actttatttg ctgctgatca acaaatagaa gcgttagtag aagccataaa atctgggaaa |
| 30961 | aagctcaaat ctgtggttat accagaagca agtaccactc accaggttgt aacaactcta |
| 31021 | tatcaattag tttacagttc gatagagtat aatcgtttgc aagaagaaaa aggattttat |
| 31081 | cgtgctagtt ttagccctga tggcaagatg ctggtttcag taagtcagga caatattatc |
| 31141 | caattgtgga gcttagatgg taagaaactt aagaaaaatt ttaccagtaa tctttcaagt |
| 31201 | gtagatagca tcaactttag tcccaacggc aaaatgctgg ctctggcagg taataatgat |
| 31261 | aattttatcg aattttggaa tctggatgga gaagataaac ccaggacgct taaaggaaaa |
| 31321 | ctttcaaaaa caaatagtgt tagctttagc cccgatggta caatgctggc tttaggaagt |
| 31381 | aacgacggca tcatccaatt atggaattta gatagtaatg aagttaaaac ttttactact |
| 31441 | aatcttaaaa taaatagtgt tagctttagc cccgatggta caatgctggc tttaggaagt |
| 31501 | aacgacggca tcattcaatt gtgggatttt cgtagcaatg aaatcaaaaa ttttaatagt |
| 31561 | aagtataaaa taaaaaatgt tagctttagc cccgatggta caatgctggc ttcagtaagt |
| 31621 | acaggaatca ttcaattgtg gagcattcaa acaggcgaaa aaatcggtgc gggaagggaa |
| 31681 | ctcgtagggg atgtggattg ggttagtcgt gttagcttca gtcaagatgg caaaatgctg |
| 31741 | gcttcaggtg gctataacgg cattattaaa atctgggaca ctgaaacagg taaagaactc |
| 31801 | ttaagcatcc aagggagtcg aggtaggact ggtgcgtcaa ttatggatat cacttttagt |
| 31861 | cctaacggca agatgttagc ttcagtagat gatgacaagt ttatcaagct ctggaatctg |
| 31921 | aatggtgaga ataaatccaa gatgctcaag ggtcagcttt caaagataag gaatattagc |
| 31981 | tttagcccca acggcaagat gttggctttg gctggtgatg ataagtttat cgaactctgg |
| 32041 | aatctagatg gacaggataa acccagaaaa cttcaactac aagtttcaaa gataaggagt |
| 32101 | attggcttta gccctaacgg caagatgctg gctttagctg gcgatgataa gtttatcgaa |
| 32161 | ctccggaatt tagatgaaca gtatgaacag tatcaacctg gaaaacttct aggacataag |
| 32221 | tcacgtgtta atagtattag ttttagccct gatggcaaga tgctagcttc agcgagcgat |
| 32281 | gaccagacaa taaaactatg gagtctagat agaaacaaag aacccacaac tttaaaaggt |
| 32341 | catagtaatt tggtcaattt tgttagtttt agtcctgatg gcaacttcct agcttcatca |
| 32401 | agtaaagacc acactatcaa actctggaga ttgaaagaca ataaggaagt ctggagtaag |
| 32461 | aaaattgggt ctggttctgg tggccaaacc tggtgggttg gttttagccc tgatagcaca |
| 32521 | atattggctt cggtgggttc ttacaatttt atccagctct ggaatctaga cggtaacgaa |
| 32581 | ttcagaaggc ttagaggtaa tctttcaaat atcaatagtg tagccttcag tcccgacagc |
| 32641 | aagatgctag cctcagtaga aacgacagaa gattcagtag gtaaagcaga ggaaagcgtc |
| 32701 | atcaaattct taaacttaga ggggcttgaa ttcaataggc tcaaaagcca atttgtagat |
| 32761 | attaatacca tcagtttcag tcctgacggc aaacttttag ctgtaggagt tgaagatggc |
| 32821 | actgttatat tacacaactt agatttagat aatttattaa gacgcggatg tcattggata |
| 32881 | cgtgattatt tgcgaaataa ccctaatgtt agtgaggtcg atcgccatct ctgtgacgat |
| 32941 | gtaccaccaa ttactacaaa tcctgagaat taggtgagtt aaaagcgaac tgcaaccacc |
| 33001 | atatgtccaa atccaagtac gaattttacc gcgaccttgc agtaatcatt gggattaata |
| 33061 | actactcaaa tggtattcca gagttagaaa cgcctgtcgc tgacgctgaa aaattcgcca |
| 33121 | aaattctcca agataattat caatatgagg cgcagatatt actaaacgaa aaagcgacat |
| 33181 | tgaaagagtt aaattctcta ctggaggact tcaagcaaaa aaaattacgc ctaccagata |
| 33241 | aaactgtaca gatagaagaa aatgaccgct taatacttta ctttgctgga cacggaatcg |
| 33301 | tcctcagaga tgggctagaa aatacggata atctggaagg atatcttgtt ccccaagatg |
| 33361 | ctagaggaga tatcttgttg caaaacaata ttcttctacc tatgcaagac ttgcatgatg |
| 33421 | cactagcaca attaccttgc cgtcatttgc taattatact tgactgctgt tttgctgggg |
| 33481 | catttcggtc gagtttgtat agagaaattg tacctgcacg aaaagtttat aaacaacggt |
| 33541 | acgatcgctt tattagagat ccagcatggc aagcgatcgc atctgcggct catgaccaaa |
| 33601 | aagctattga ccatctaggt tgttttggac agagaggaag caaaggcaaa cactctcctt |
| 33661 | ttgcaaaagc tttgtttgac ggtttgctgg gtgctgctga cacaactcta agagaaggtg |
| 33721 | atggtattat cacagcaaca gaactttact gttatttacg cgatcgcgtt gaggaactaa |
| 33781 | ctgataaata tgacaaacgc caaactccag gtttatttcc tctgaaaaag catgataaag |
| 33841 | gcgagtatat cttcttgctt cccaacttcg accaaaacaa tttagaagat gcaccagcac |
| 33901 | tcaagcaaga aaacaatcct tatcggggtt taaagagcta tgaagaagaa cattctctcc |
| 33961 | tgttttttgg tagagatgaa cttgtcaaag aactatctac ccatgtttct caaaccaata |
| 34021 | acccactcac cgtagttttg ggtatttctg gttctggaaa gtcgagctta gtgaaagcag |
| 34081 | gactcattcc ttatttacga aaacatcata accaagaatg gtacatcata ccaactattc |
| 34141 | gcccaggaga taattctttc acttctctgg cagaggcact tgtagaaaag tctgaaacag |
| 34201 | atggaaatta cttaaacaaa attaaatctc taaatcagac attaaaacaa ataccacagg |
| 34261 | attttattaa gataatacaa gaattactac taatacctga aaaagtaaaa attttattag |
| 34321 | ttatagacca atttgaagaa ctaatcacag tttgtaatcc acaagaaaaa gaacaatttt |
| 34381 | taaatttcct tagcagtatt ttagaagctc atcctcagca aatttccatt gtattgactc |
| 34441 | tgcgttctga ctttgaacct cggtttttag attctgctct gaaagcatat tggacacaat |
| 34501 | ctcgttttcc tatacgagct atgagatccg atgaattacg gcaggcaatt gaacgacctg |
| 34561 | ctaacgaaaa aatgctcgat tttgacccac caaatttagt tgatcagttg attgacgaag |
| 34621 | tggggcaaat gccaggagca ttgtcgctat tatcttttac attgagtgaa ctatatatta |
| 34681 | aagccataaa tcgagaaagc agaactttaa gtaagactga ttacgataca ttaggcggag |
| 34741 | ttgcaggttc attaacacaa agagcaactc aggaatacca aacattagtg aaacttgatc |
| 34801 | cagcttatga acataccgtc cgccaactca tgctgagaat ggtagctatt gaaggtaatg |
| 34861 | aattagcacg acgacgagta ccaatgtcag aactggagta tgagagtgta gaaaaaaatc |
| 34921 | aaagggtgtc gcgggcgatt gctcagtttt cggcggcacg tctaattgtt gaaggtaaag |
| 34981 | aaactggtga cgaaccttat gttgaaccag cacacgatgc cttagtaagc gggtggaaca |
| 35041 | aactacagga atggaaaaat gatgagcaag gaagtttagt tttgcgtcaa cgcttaacac |
| 35101 | catctgctaa cgattggcta aaagatgggc gcaaatctga ttatctatgg attcgtgatc |
| 35161 | ctcgcctcgc tatcctggaa aaagtcctgg aattgccatc tgaagaaaac tggctcaatc |
| 35221 | agctagaaac agaatttgtg aacaccagta ttcagaaaag aaaggatgaa cttgaagaaa |
| 35281 | ctaaagaaca actccgcatt tctgaagagc gtcgtatcga agcagaaaag caaactgcga |
| 35341 | tcgcgcttgc tagaaattct gaagccttac tatcttctaa tcaacaatta gaagcactga |
| 35401 | ttgcagcagt aaaggcagga agaaaattac aaaatctact agcagaagaa gctatttctt |
| 35461 | cacaaagttt gttattcaag caaactgtgt tattcaagca aactgtgatt gccctagaac |
| 35521 | gagcagttta tggagtgaga gagcgtaacc gtttggaagg acatagcggg ccagttcaaa |
| 35581 | gcgtctgttt tagtcctgat ggcaagatga ttgccactgc tagttgggat aatacagtta |
| 35641 | agctatggag tagggaaggt aaagaactaa aaactctgaa aggacattat gatggtgttt |
| 35701 | taagtgtctg tttcagtcct gatggtcaaa ctgttgcctc aactggttgg gataacacga |
| 35761 | tcagaatatg gaatctggag ggacaggagc ttacacagca ccctctcgga tttggccata |
| 35821 | cagatcaggt atggtgtgtt tgttttagtc ctgacggaga aatgatcgcc tctgccagtc |
| 35881 | atgacaggag agttaaactg tggaataaag atggacagtt tctcagaaac ctttgtgaat |
| 35941 | caagtaccac cgagcatgaa ctaaccgtca gtagtgtttg ctttagtcct gatggaaaaa |
| 36001 | taattgctac tgcaagttat gataacacag ttaaactttg gaatcttgac ggtactttaa |
| 36061 | ttaaaagttt tacacatagc ggggcagttt atgatatcag tttcagcccc gatggtaaca |
| 36121 | ctattgtttc tgctagtgca gacaagacag taaaaatttg gagtttaaac ggcaaaaaac |
| 36181 | aaactcttaa aggacatgaa ggtgaattta cgagtgttag gtttagccct gatggaaaaa |
| 36241 | taattgcctc tggtagtaaa gataattcta tcaagctttg gagtaaagac ggtcaggaac |
| 36301 | ttacaactct cagagggcat actgcggaaa ttactagcat taattttagc cctgatggta |
| 36361 | atatcattgc ttctgctagt gaagattcaa ctgtcaagat ttggaacaaa gacggtcaac |
| 36421 | aaatcactat ccttccagaa tatggtgaag gggttactca agtctgtttt agtccagacg |
| 36481 | gcaaaacaat tgttggtaat gcgaaaaata cactcaaact ctggaatctt gatactcaag |
| 36541 | agagcaaaat gttttctgat attactggtg caaacaatgg tatcagtttc agttttagcc |
| 36601 | cagatggtaa cgcgatcgcg gtctgtggag gttttgacaa aatggttaaa ctttgcaatc |
| 36661 | ttgaaggcca agaaattaag aactctaacg attattatca cgcaataagg gctatccggt |
| 36721 | tcagccctga tggtaaaact attgctttta ataataataa aaagattgac ctttggaatt |
| 36781 | gggagaatca agagataaaa accctgtcag ggcatacagg caaggtttgt agtctatgtt |
| 36841 | tcagcccaga cagtaagata attgtttctg gtagtgatgg ttctgataag acaattaaac |
| 36901 | tgtggaacct aagcagtggt gaagttatca tccttgaagg tcatagtaat agtgtaaatc |
| 36961 | aagtctgttt cagtccagat ggcgaaataa ttgcttctgc cagttctgat aacacagtaa |
| 37021 | aactttggaa tcttaatgga caagaactaa aaactctccg aggtcatagc tatcaggtat |
| 37081 | ttgatgtaac ttttagcctt gatgggaaaa tgattgcttc tgctgattgg gatggcacaa |
| 37141 | tcaaactttg gaatcgtgat ggtcaactgc tcacaacttt agaaaaacga agtgaaaaac |
| 37201 | tatccagtat tagcttcagc ccggatgttc aaatcctggc tgctgccagt aataaaggag |
| 37261 | taattttatg gaacttcaat ttagatgact tagtagcacg tggttgtaat ttgttacatg |
| 37321 | aatacttaat gaataacact aatgttcaag aagagcaaaa tctctgcaat gacagatgaa |
| 37381 | taagcagtaa gcaatatacg caaaatagtt cacagaaaat aactgacatt ctcaaaacta |
| 37441 | aattagttat taacccaagt tgctagttat gccgaagagc gatcgcagtg ggttccattg |
| 37501 | ccgcttcaga atggatagtt aacatatact ctcaaggcga ctgtatttac tgcttccctg |
| 37561 | cgtggcgtag ccattcctgt tacttaatgt ttccggtttt agtcgatact attaccatag |
| 37621 | cagcaataga gggattactg attaagcttg ctctggagag cgatcgcaag ttattttgga |
| 37681 | acaaatagat gccaaatcct tacagggctt gctttcggca aattatatga cttttttatt |
| 37741 | tatcaatatg aggcatttct gaaaaattgt acaactttat tttttgatat caaataaaat |
| 37801 | tgaacgtaaa tacaaggctt ataaactttc aaattatata agttttttat gtttttacaa |
| 37861 | taatttggta gaataaccag ataatttctg aatgtgatga taatattatc acaatcagga |
| 37921 | atattttttt gggcgatcgc ctacggtgct gatgcgtcca cctcatcact tgtctataaa |
| 37981 | taaaaaacaa gcaggacata tactatgagc caaaaaacca ttgattccaa aaatctgacc |
| 38041 | atagaaaaaa tttttgaaga tttttatatt attcctagtt atcaaagaga atatatttgg |
| 38101 | aaagaaaaac aagtattaga gttattaaat gatgtatata gtgaatttcc tggctcttta |
| 38161 | aatatagaat ctgatgaata ttttctaggc agtattgtaa tttgtcctag ttctgaagat |
| 38221 | acagacaatg tgtatcaggt aattgatggg caacaaagat taacaacttc ttatatcttc |
| 38281 | ttttgtgctg tcaaaaatta tataaataag ttgccagaac atcaaaagtt tgatgcgatt |
| 38341 | aagaaattaa tttattcaga gtatacaaat gcacaaggtc aagaccaatc acaatacagg |
| 38401 | ataaatcctc aatatccaga cagtaaaaat attttggaag aatttgctaa agatgaaatc |
| 38461 | aatatagaag agatagaaaa taaaaaaaat aataaaactg aatctacaat taatttgatt |
| 38521 | tcagcttacc aaacaataga agattttatt aaggataagt tcaaagatga tgaaaaccag |
| 38581 | atcaaaaggt tttatgcaca tttcatcaaa aaagtaaagt tagttagagt tgaaactaca |
| 38641 | gatattaacc acgccttaag agtttttgaa accatcaata atagaggcgt tggcttggat |
| 38701 | tctatggatt tacttaaaaa tcttttgttt aaacaagttg aaaaagaaga tttttcaaaa |
| 38761 | attagtaatt cttggaaaga aataaaaaat gaattagatt cagtaaagga aaagcctatg |
| 38821 | gattttctac gatactttat tctaactcaa tatgatgttg aaagagataa tgtccaaact |
| 38881 | aaagaatacc aatggttact ggaaaataat actacttgta aatatgaaga aaaaccattt |
| 38941 | gagtttgtcg aggacttact gaaggctgca aaggcttata aatcattact agaaagaaaa |
| 39001 | aatccttctg ataattcaga taacccgcat ttagctaata ttaagtattt aagccccaat |
| 39061 | cttaaacaac atctatatat actgttagca gcaagggata tgtcaatgga aatattttct |
| 39121 | caattgtgtc tacatattga aaactttatt tttgtttata gttttcctaa taattcaatc |
| 39181 | agtgaattag aaaaaatatt tattaattgg ggtcaagaga ttcgtaaaat agtacaaaat |
| 39241 | acagacaaag aagagcaatt atctaaatta aaagaatttt tatataacaa tctaggtgta |
| 39301 | cgtaaagaaa atctcaagaa atcttttgag aaaaagttta aggaagcaag tcaattagac |
| 39361 | tttaattcaa gaggaacaaa taaaaagcca acatcaaatg ctaataaaaa aacaaagtat |
| 39421 | atattagcaa aaatttctca gtatgtacaa cttatagctt atagcgacaa tgatgagtgt |
| 39481 | aaaaatttta caacttttac tcagagttca atagaaattg agcatatttt accacaagat |
| 39541 | ttaaattctg aacctgttaa agcttttgat aaaccagatg aagttgaatc atatatatac |
| 39601 | aaattaggca atttagcttt gctagaacaa actctcaatg cttctattca aaatagtcca |
| 39661 | tttgaagtta agaaagaagc ttatcaacaa tcgaattttc ttttaacaca atgtatatct |
| 39721 | gaaaagccca ctattggaac taatactgca attaataggg ctgtggagaa attaataagt |
| 39781 | tttaatgaat ggaattcaca atcaattgat aaaagacaag aaatgctcac agaattagca |
| 39841 | ttgaaaatat ggtacgagga agtatctttc gaagaagtac cttttgagga aagtctggat |
| 39901 | gctagggaaa tacaggaact attgcggttt ggtggtgaaa gagctattta atttgatagc |
| 39961 | atctgatatc ctttctacac ctaaagtgcc agttgcgtaa gtcctgaaca tatatagata |
| 40021 | gaaagtgaga taagaacagg taagagtaag ctattcaaac acaacgccaa ttttcgattt |
| 40081 | gtaagttagg tattctttga aattctcgaa cattagatgt caccacaata tgattattga |
| 40141 | tgactgcggt agctgcgatg agtacatcat acgcaccaat aggtgaacca gctagtttga |
| 40201 | gaaagcttct gatttcggct gcttgttctg ctgctttcga gtcaaaaggt aaaattgtga |
| 40261 | ttgagtttaa taatgtttca aaaattgatt ggattttaac agcgcgttgt gggttaatag |
| 40321 | ctaatccata tttcacctcc ataactgtta gagatgacac aaaaatctcc gcaggggaaa |
| 40381 | ttaattttat tcgcttgagg gtgttttctt ctcctttgac aaaatcacta atgacgcaag |
| 40441 | tatccaatag ataacccata cataattaaa gttctatttc gttaggaggt atcagttcat |
| 40501 | ctcgatatga ttcaaagata atactttcgt ttacaccttg atattgtatt agggtttctg |
| 40561 | gccattggct gggtttagtt tctaggaatg tcacgaaaac aatgctttca gtaatacctt |
| 40621 | gcggtgtttc agctagttca attttgccgt ttttataaat tccttgaatg gtttttaaca |
| 40681 | tagagcttac ctcctacatt aagtttaacc tatttattct ccttcttcag tcatgctggg |
| 40741 | tgggaatcga atgtctgagt cttcaatata aattttacct gagatgagac gatttatcag |
| 40801 | gcgatcgcgt gtttgtttaa ggtgtaattt tactaattgg atataatcaa tttgattgtt |
| 40861 | caatttactt gcaaatttac aaaagtttac tgtgt |

Features of the components in the D318 locus above, e.g., TnsA, TnsB, TnsC, TniQ (TnsD), Cas5, Cas6, Cas7, Cas8, and CRISPR clusters are shown below.

FEATURES Location/Qualifiers

| | |
|---|---|
| CDS | 265..510 |
| | /product = ″NA >cl_18381″ |
| | /gene = ″NA >cl_18381″ |
| | /locus_tag = ″NA >cl_18381″ |
| | /label = ″NA >cl 18381 CDS″ |
| CDS | 735..2609 |
| | /product = ″NA >cl_19425″ |
| | /gene = ″NA >cl_19425″ |
| | /locus_tag = ″NA >cl_19425″ |
| | /label = ″NA >cl 19425 CDS″ |
| glmS | 735..2609 |
| | /created_by = ″makoto″ |
| | /label = ″glmS″ |
| ITR | 2634..2700 |
| | /created_by = ″makoto″ |
| | /label = ″Right End″ |
| motif_3 | 2642..2660 |
| | /Mismatches = 0 |
| | /%_Identity = 100 |
| | /Motif = ″[AC][AC][TA]AA[AT]AAAGT[TC][AG]TA[CT]AN[TC]″ |
| | /annotation_group = ″AttTn7_cluster_2: 7,354,202 -> |
| | 7,354,220″ |
| | /label = ″AttTn7_cluster_2″ |
| motif_3 | 2662..2680 |
| | /Mismatches = 0 |
| | /%_Identity = 100 |
| | /Motif = ″[AC][AC][TA]AA[AT]AAAGT[TC][AG]TA[CT]AN[TC]″ |
| motif_3 | /annotation_group = ″AttTn7_cluster_2: 7,354,222 -> |
| | 7,354,240″ |
| | /label = ″AttTn7_cluster_2″ |
| | 2682..2700 |
| tnsA | /Mismatches = 1 |
| | /%_Identity = ″94.73684210526316″ |
| | /Motif = ″[AC][AC][TA]AA[AT]AAAGT[TC][AG]TA[CT]AN[TC]″ |
| | /annotation_group = ″AttTn7_cluster_2: 7,354,242 -> |
| | 7,354,260″ |
| | /label = ″AttTn7_cluster_2″ |
| | 2742..3557 |
| CDS | /product = ″1-272\|id:63.235@bs:384″ |
| | /gene = ″1-272\|id:63.235@bs:384″ |
| | /locus_tag = ″1-272\|id:63.235@bs:384″ |
| | /label = ″1-272\|id:63.235@bs:384 tnsA″ |
| | 2742..3560 |
| tnsB | /product = ″tnsA_10 >cl_27484″ |
| | /gene = ″tnsA_10 >cl_27484″ |
| | /locus_tag = ″tnsA_10 >cl_27484″ |
| | /label = ″tnsA 10 >cl 27484 CDS″ |
| | 3581..5809 |
| CDS | /product = ″1-741\|id:67.738@bs:1002″ |
| | /gene = ″1-741\|id:67.738@bs:1002″ |
| | /locus_tag = ″1-741\|id:67.738@bs:1002″ |
| | /label = ″1-741\|id:67.738@bs:1002 tnsB″ |
| | 3581..5812 |
| CDS | /product = ″tnsB_11 >cl_26522″ |
| | /gene = ″tnsB_11 >cl_26522″ |
| | /locus_tag = ″tnsB_11 >cl_26522″ |
| | /label = ″tnsB 11 >cl 26522 CDS″ |
| | 5802..7475 |
| tnsC | /product = ″NA >cl_6196″ |
| | /gene = ″NA >cl_6196″ |
| | /locus_tag = ″NA >cl_6196″ |
| | /label = ″NA >cl 6196 CDS″ |
| | 5832..7472 |
| tniQ | /product = ″18-568\|id:62.976@bs:712″ |
| | /gene = ″18-568\|id:62.976@bs:712″ |
| | /locus_tag = ″18-568\|id:62.976@bs:712″ |
| | /label = ″18-568\|id:62.976@bs:712 tnsC″ |
| | 7483..8445 |
| | /product = ″1-315\|id:40.683@bs:231″ |
| | /gene = ″ 1-315\|id:40.683@bs:231″ |
| | /locus_tag = ″1-315\|id:40.683@bs:231″ |
| | /label = ″1-315\|id:40.683@bs:231 tniQ″ |
| CDS | 7483..8430 |
| | /product = ″NA >cl_2402″ |
| | /gene = ″NA >cl_2402″ |
| | /locus_tag = ″NA >cl_2402″ |
| | /label = ″NA >cl 2402 CDS″ |
| cas6 | 8603..9283 |
| | /product = ″1-227\|id:90.308@bs:421″ |
| | /gene = ″1-227\|id:90.308@bs:421″ |
| | /locus_tag = ″1-227\|id:90.308@bs:421″ |
| | /label = ″1-227\|id:90.308@bs:421 cas6″ |
| CDS | 8603.. 9286 |
| | /product = ″NA >cl_11380″ |
| | /gene = ″NA>cl_11380″ |
| | /locus_tag = ″NA >cl_l 1380″ |
| | /label = ″NA >cl 11380 CDS″ |
| cas8 | 9304.. 10839 |
| | /product = ″1-512\|id:88.867@bs:952″ |
| | /gene = ″ 1-512\|id:88.867@bs:952″ |
| | /locus_tag = ″1-512\|id:88.867@bs:952″ |
| | /label = ″1-512\|id:88.867@bs:952 cas8″ |
| CDS | 9307.. 10803 |
| | /product = ″devT_12 >cl_6205″ |
| | /gene = ″devT_12 >cl_6205″ |
| | /locus_tag = ″devT_12 >cl_6205″ |
| | /label = ″devT 12 >cl 6205 CDS″ |
| cas7 | 10901..11872 |
| | /product = ″1-324\|id:90.123@bs:493″ |
| | /gene = ″ 1-324\|id:90.123@bs:493″ |
| | /locus_tag = ″1-324\|id:90.123@bs:493″ |
| | /label = ″1-324\|id:90.123@bs:493 cas7″ |
| CDS | 10901..11875 |
| | /product = ″devR_22 >cl_17591″ |
| | /gene = ″devR_22 >cl_17591″ |
| | /locus_tag = ″devR_22 >cl_17591″ |
| | /label = ″devR 22 >cl 17591 CDS″ |
| cas5 | 11877.. 12506 |
| | /product = ″1-210\|id:89.048@bs:396″ |
| | /gene = ″1-210\|id:89.048@bs:396″ |
| | /locus_tag = ″1-210\|id:89.048@bs:396″ |
| | /label = 1-210\|id:89.048@bs:396 cas5″ |
| CDS | 11877..12509 |
| | /product = ″devS_14 >cl_25717″ |
| | /gene = ″devS_14 >cl25717″ |
| | /locus_tag = ″devS_14 >cl25717″ |
| | /label = ″devS 14 >cl 25717 CDS″ |
| CRISPR | complement 12786..13107) |
| | /product = ″5 ACTGCTCAACGTCTAACGACATCTAATGTTAAAGCAC″ (SEQ ID NO: 66) |
| | /gene = ″5 ACTGCTCAACGTCTAACGACATCTAATGTTAAAGCAC″ (SEQ ID NO: 67) |
| | /locus_tag = ″5 ACTGCTCAACGTCTAACGACATCTAATGTTAAAGCAC″ (SEQ ID NO: 68) |
| | /label = ″5 ACTGCTCAACGTCTAACGACATCTAATGTTAAAGCAC CRISPR″ (SEQ ID NO: 69) |
| CDS | complement(13305.. 13535) |
| | /product = ″NA >cl_2538″ |
| | /gene = ″NA >cl_2538″ |
| | /locus_tag = ″NA >cl_2538″ |
| | /label = ″NA >cl 2538 CDS″ |
| tniQ | 13829..15736 |
| | /product = ″1-636\|id:91.038@bs:1200″ |
| | /gene = ″1-636\|id:91.038@bs:1200″ |
| | /locus_tag = ″1-636\|id:91.038@bs:1200″ |
| | /label = ″1-636\|id:91.038@bs:1200 tniQ″ |
| CDS | 13829..15739 |
| | /product = ″NA >cl_1243″ |
| | /gene = ″NA >cl_1243″ |
| | /locus_tag = ″NA >cl_1243″ |
| | /label = ″NA >cl 1243 CDS″ |
| CDS | complement(15782.. 16087) |
| | /product = ″NA >cl_14703″ |
| | /gene = ″NA >cl_14703″ |
| | /locus_tag = ″NA >cl_14703″ |
| | /label = ″NA >cl 14703 CDS″ |
| CDS | complement(16245.. 16538) |
| | /product = ″NA >cl_2353″ |
| | /gene = ″NA >cl_2353″ |
| | /locus_tag = ″NA >cl_2353″ |
| | /label = ″NA >cl 2353 CDS″ |
| CDS | 16744..18837 |
| | /product = ″NA >nocl″ |
| | /gene = ″NA >nocl″ |
| | /locus_tag = ″NA >nocl″ |
| | /label = ″NA >nocl CDS″ |
| CDS | 18834..20024 |
| | /product = ″NA >nocl″ |
| | /gene = ″NA >nocl″ |
| | /locus_tag = ″NA >nocl″ |
| | /label = ″NA >nocl CDS″ |
| CDS | 20158..23391 |
| | /product = ″NA >cl_16927″ |
| | /gene = ″NA >cl16927″ |
| | /locus tag = ″NA >cl16927″ |
| | /label = ″NA >cl 16927 CDS″ |
| CDS | 23553..27491 |
| | /product = ″NA >cl_9573″ |
| | /gene = ″NA >cl_9573″ |
| | /locus_tag = ″NA >cl_9573″ |
| | /label = ″NA >cl 9573 CDS″ |
| CDS | 27481..28449 |
| | /product = ″NA >nocl″ |
| | /gene = ″NA >nocl″ |
| | /locus_tag = ″NA >nocl″ |
| | /label = ″NA >nocl CDS″ |
| CDS | 28501..32973 |
| | /product = ″tolB_48 >cl_2719″ |
| | /gene = ″tolB_48 >cl_2719″ |
| | /locus_tag = ″tolB_48 >cl_2719″ |
| | /label = ″tolB 48 >cl 2719 CDS″ |

CRISPRcomplement(31332..31631)
/product = ″3ATGCCGTCGTTACTTCCTAAAGCCAGCATTGTACCATCGGGGCTAAAGCTAACACTATT T″ (SEQ ID
NO: 70)
/gene = ″3ATGCCGTCGTTACTTCCTAAAGCCAGCATTGTACCATCGGGGCTAAAGCTAACACTATT T″ (SEQ ID
NO: 71)
/locus_tag = ″3ATGCCGTCGTTACTTCCTAAAGCCAGCATTGTACCATCGGGGCTAAAGCTAACACTATT T″ (SEQ
ID NO: 72)
/label = ″3ATGCCGTCGTTACTTCCTAAAGCCAGCATTGTACCATCGGGGCTAAAGCTAACACTATT T CRISPR″
(SEQ ID NO: 73)

| | |
|---|---|
| CDS | 33003..37379 |
| | /product = ″tolB_47 >cl_2719″ |
| | /gene = ″tolB_47 >cl_2719″ |
| | /locus_tag = ″tolB_47 >cl_2719″ |
| | /label = ″tolB 47 >cl 2719 CDS″ |
| ITR | complement(37722.. 37859) |
| | /created_by = ″makoto″ |
| | /modified_by = ″makoto″ |
| | /label = ″Left End″ |
| motif_3 | complement(37722.. 37740) |
| | /Mismatches = 0 |
| | /%_Identity = 100 |
| | /Motif = ″[AC][AC][TA]AA[AT]AAAGT[TC][AG]TA[CT]AN[TC]″ |
| | /annotation_group = ″AttTn7_cluster_2: 7,389,282 <- |
| | 7,389,300″ |
| | /label = ″AttTn7_cluster_2″ |
| motif_3 | complement(37766.. 37784) |
| | /Mismatches = 0 |
| | /%_Identity = 100 |
| | /Motif = ″[AC][AC][TA]AA[AT]AAAGT[TC][AG]TA[CT]AN[TC]″ |
| | /annotation_group = ″AttTn7_cluster_2: 7,389,326 < |
| | 7,389,344″ |
| | /label = ″AttTn7_cluster_2″ |
| motif_3 | complement(37833..37851) |
| | /Mismatches = 1 |
| | /%_Identity = ″94.73684210526316″ |
| | /Motif = ″[AC][AC][TA]AA[AT]AAAGT[TC][AG]TA[CT]AN[TC]″ |
| | /annotation_group = ″AttTn7_cluster_2: 7,389,393 <- |
| | 7,389,411″ |
| | /label = ″AttTn7_cluster_2″ |
| CDS | 38005..39951 |
| | /product = ″NA >cl_7816″ |
| | /gene = ″NA >cl_7816″ |
| | /locus_tag = ″NA >cl_7816″ |
| | /label = ″NA >cl 7816 CDS″ |
| CDS | complement(4005 5 ..40459) |
| | /product = ″NA >cl_13569″ |
| | /gene = ″NA >cl_13569″ |
| | /locus_tag = ″NA >cl_13569″ |
| | /label = ″NA >cl 13569 CDS″ |
| CDS | complement(40466. .40681) |
| | /product = ″NA >nocl″ |
| | /gene = ″NA >nocl″ |
| | /locus_tag = ″NA >nocl″ |
| | /label = ″NA >nocl CDS″ |

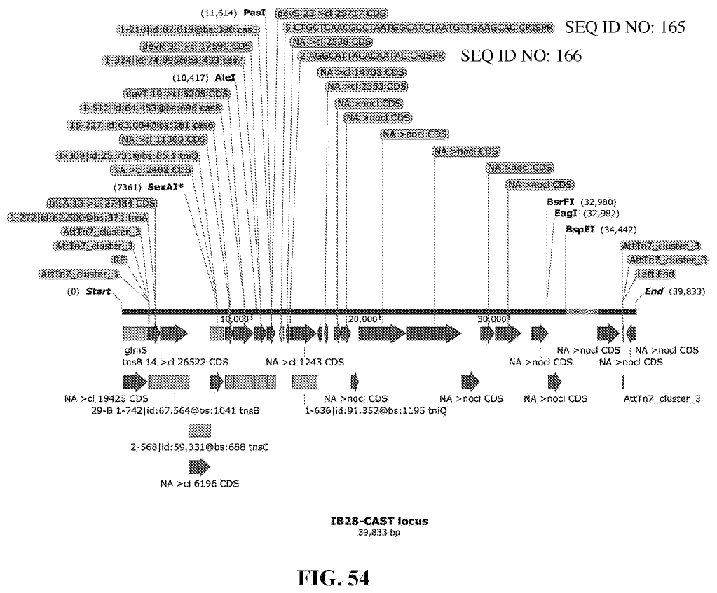

The map of IB28 locus is shown in FIG. 54. The sequence of IB28 locus is shown below.

| IB28-CAST locus (39,833 bp); Accession: | |
|---|---|
| 133464\|0\|GCA_002368215.1_ASM236821v1_genomic\|AP018184.1\|6522801\|Nostoc | |
| (SEQ ID NO: 74) | |
| 1 | ttataaaaat acctagtggc caaaggagca aacctgctcc ttttttcttt ggtaaagttt |
| 61 | gttgatgatt aacggttgac tgtggactat ggactattga ctatgaactg ttgactatac |
| 121 | tggaatttaa ttctcttgaa gaaagggtgg tgtgagcaat gtgtggcatc gttgggtata |
| 181 | ttggcactca agcagcgaca gaaatcttat tatctgggct ggaaaagtta gagtatcggg |
| 241 | gttatgactc agcaggtgta gctacgcttt gggaaggtga ggttcactgt gtccgagcca |
| 301 | agggcaaact gcataacctc cgttctaaac tagagcaact agtaacctcc tctcaaatcg |
| 361 | gaattggtca tacacgctgg gcgactcatg gtaaaccaga ggagcataac gcccatcctc |
| 421 | atttagatac agcaaggcgc atagctgttg tccaaaatgg gattattgaa aattaccgcg |
| 481 | agttacggga agaactgaag cagaaaggat atgagtttcg ttctgaaacc gatacagaag |
| 541 | ttattcctca cctcatagcc gaattgtcaa aaaatgcctc ctcttttcta gaagcagtac |
| 601 | gccaagctgt tagccatctg catggagcat ttgcgatcgc cgtcatttcg gctgattacc |
| 661 | ccgacgaatt gattgttgtc cgccagcaag cgccgttagt gataggattt ggtcaaggtg |
| 721 | agtttttctg tgcttcagat acgccagcca tagtttccca tacccgcgcc gtcttacccc |
| 781 | tggagaatgg cgaaatcgcc cgtcttacac ccttgggagt agagatttat aactttgctg |
| 841 | gcgacaggct caaaaaacaa ccccggatgc tcaacttgag tcccacgatg gtagaaaagc |
| 901 | agggattcaa gcactttatg ctcaaggaga tttatgagca accaggagtt gttagagcaa |
| 961 | gtttagacgc atactttaat acagaggtta attctggtaa atcattccaa tcaccagtta |
| 1021 | agttaggttt acccgaacag ttctacgcag acttagaaca aattcatatt gttgcttgcg |
| 1081 | gtacaagttg gcacgccgct ttagtaggga agtatttact tgaacaatta gcaggaatct |
| 1141 | ctactcaggt acattatgct tctgagtatc gttatgcacc atcaccgcta acagcgaata |
| 1201 | cattaattat tggcgttacg cagtctgggg aaacagctga taccttagca gccttagcaa |
| 1261 | tggaaaaaga acgccgtcaa ggtaaagaag caaaatatca agcgcaattc ttgggtatta |
| 1321 | ccaatcgccc agagagtagc cttggtcatc ttgtacccca tattatcaac accttggcag |
| 1381 | gaattgaaat cggtgtggca gcaactaaaa cctttgtcgc ccaactgatg gcgttttatg |
| 1441 | ccctagcctt agatttagcc tatcatcgtc aaactgtagc gccggataaa ttagcagata |
| 1501 | ttattcaagg tttgcgccag atacccaagg aaatagaaga aaccttagaa cgccaagaaa |
| 1561 | aattaacgga acatttagct catgaatttg cagagactca agacttcatc tttttgggta |
| 1621 | gagggattaa tttccccatt gcattggaag gggctttgaa attaaaagaa attagctata |
| 1681 | ttcacgctga aggttatccg gctggggaaa tgaaacatgg ccccattgct ttattagatg |
| 1741 | caaaagtgcc agtagtggcg atcgctttcc ctggtactgt gtatgagaaa gtcatttcca |
| 1801 | actctcagga agccaaagcc agagattccc gcttaattgg cgtaacgcca gtcagcgatg |
| 1861 | gcgaagccgg ggaaatcttt aatgatttac ttcccgtttc ccctgtggat gaattacttt |
| 1921 | cacccatcct cacagtagta cctttgcaac tattggctta ccacatcgcc gcccgtcgcg |
| 1981 | gtttggatgt cgatcagcca aggaatcttg ctaagagcgt tactgttgag tagtgttgaa |
| 2041 | tcgagaaata ttgaggttgt ggaaacataa taaagttgta caataaataa taaagttgta |
| 2101 | caatgcctaa taaagttata caatttctcc tatgggtgag tataattcca tctgtaggag |
| 2161 | attgtcttta tggctagaac caagcaagag tggacgcaag ctaagtttga gcgctacatg |
| 2221 | aaggaaggtc gaggtcaggg tagtggaaaa aattatcaac cgtggataaa aattcaagat |
| 2281 | tttccgtcga agggtagggt atctagacca cccggttgga aaacaaaccg agagcatcat |
| 2341 | ctgttctccg atcatgagaa acggttattt tatttgtttg agtggtcgga cagcattatt |
| 2401 | gatataaggg aacaattccc tttaattgac ttagacttag cgatgaacat tgctaccgaa |
| 2461 | atggcgattg agtatcccaa agatgttaat aataatactc cctatgtttt aactacagac |
| 2521 | tttatgttat ctgtgaagca aggaaagcag atagttgaaa aagctcgcac tttcaaatta |
| 2581 | gttaaggatt taggaattaa atctgttgct gaaaaatttg agttagaaaa gcgttattac |
| 2641 | acagcaaaag ggattgattg gggtattatt acggaaaaag aaattcctac gcttttagta |
| 2701 | aaaaatgttg agtggattca ttctagttat aagttagaag cagccgcaga aatcaatgtt |
| 2761 | gatgagttga ataacatcgc aaatatttta aagtcaaaat tgcaagaaag cgattctact |
| 2821 | attaataaaa ttacaaattg cttagataaa gagatgaatc tagaaatagg tacgtcactc |
| 2881 | tacattttta agcatctcct tgccaataag aaaataatca tggatatgca aacaaatgca |
| 2941 | atatctagtt ctatttcagc gcgagaaatt caaaaaatca ttttctagaa atttatattg |
| 3001 | gttgttccca tgaataataa tttgtttgtt aatgacttaa ttgagtggat tgacgaatca |
| 3061 | ggaaacaact ttgtagagag aattatttgg attgatgaag cctatatcat tgcctttacg |
| 3121 | atagatataa atatcaaaac tggatttcca gtgtctaaaa gagtatcaga tatccaagaa |
| 3181 | gcaatttatg aaggtcgtgc attgaagttg aggacagacc cttgggcaag aatcgttaga |
| 3241 | gatgaagact tatctgctaa agaaaaagaa attagagata agtattggga tattatttca |
| 3301 | tctatagtaa ttcaagaacc atctatatac tatcgagata aacgaggctc gctcattaaa |
| 3361 | caagttatag aaaaatataa taataataag agcgaaggaa aattaattga aagaagcgtt |
| 3421 | tatcagatta ttaggagata ctggcaaaga ggtaaagata aaaactctct cttacctgat |
| 3481 | tatgcaaatt ccggtggcaa aggcaaagta agagcatcta gagaaaaaaa gagaggaaga |
| 3541 | ccaagaaaat atgctcatga gccagacatc ggtgtaggta taaatataac cgatgaagat |
| 3601 | aagaaaatat ttagattagc agttactaag ttttatcaca atcaaaaaga aaactttttg |
| 3661 | acaactgctt atgatttaat gattaaaaac tattattcag aggaaactat ttacgatgag |
| 3721 | aatggcgtta aaaaacatat attaattccc ccagataaac gaccaacatt aacccagttt |
| 3781 | aaatattggt atgacgttga acaaggagac attagaaaaa aaataatttc tcgtagggga |
| 3841 | gctaaaaaat atgctctgga acatagagca attacaggaa actcaacaat ggaaactatt |
| 3901 | gggcctggtt ctagatacca aatagatgcg acgatcgcag acgtgtacct agtttctgcg |
| 3961 | tacaatcgca actggattgt tggtagacct gttatctatg tagtcatcga tgtttttagc |
| 4021 | cggatgataa caggagttta tgttgggttt gaaggccctt cttggatagg agcaatgatg |
| 4081 | gctttagcaa atgcagccag tgataaagta gcattttgca aagaatacgg tatcgagatt |
| 4141 | acagaagatg aatgggcttg tcatcatatt cctgatgcaa ttttaggcga tcgcggcgag |
| 4201 | ttagcaggga tggcagtaga aacactaatt cccaatcttg gtattcgtat cgaaaatgct |
| 4261 | gcaccctacc gagcagattg gaagggttta gtagaaagac attttggcat tatccacggt |
| 4321 | catatcaaac catttgtgcc aggttacata gacaaggatt ttagggaaag aggagcaaga |
| 4381 | gattatcgac ttgatagcaa gcttgatata gagcagttta ctgaaattat tatcaagatc |
| 4441 | atcctttacc acaacaatga acattactta gataactatg aaagagatga ggcaatgata |
| 4501 | gctgatgatg ttccccccat acctagagaa ttatggcagt ggggaattgc aaatcgttca |
| 4561 | ggtagactta ggactattcc tgaagatatt gttaagctga acttaatgcc aacagaaaac |
| 4621 | gcaacgatta ccgaacgtgg cattaaattt aaaggaatgt actacacctg tcaaaaagca |
| 4681 | aaaaacgaat tttggtttga gaaagctaga agtaattctc tttctaggtt agagaaaaaa |
| 4741 | ttagaaattt catatgacat tagaaaacct gattatatat atcttcgctc tcctgatggt |
| 4801 | agaaactttg agaaatgttt tcttttagaa tcagaatcaa aatattttaa taaaaattta |
| 4861 | catgaaattg aatatttatt tgcttttgaa gagttacaaa aacaaaaaaa taaaggttta |
| 4921 | aaacaacaac agaaagtaga cttaatcgca gaaattgaaa gtgtcgtgaa taaagcaaaa |
| 4981 | aaaatgaccg aggaaatgaa agatgataaa ctgagcaata gaaaaagaac cgcaggtatt |
| 5041 | cgagataata gaagtgctga aaaatctaaa cgtcgaaaaa ttgaaggctt tgagctagaa |
| 5101 | aagagtgatc atgaaaatac tagtaatact gtggatattg aaagtcaaaa agtagagcag |
| 5161 | ccacaatcat tgcaacctaa ctatcttgat attcttcgtc agaagcgaga ggaacgaaag |
| 5221 | cgtgaacaag ataaatgatt ggaaatgggt tgaaattcca aatggtaaat ctgctgctgt |
| 5281 | agctaaatat aatgaccata aattaccaga gtacaataat aaccctttaa ttcaagcatt |
| 5341 | acctccaatt ctttctggag aagaatttat tgaaaaagca ataatactcc ccagttacgc |
| 5401 | acctgaagag agggaaatgg atgcacaccg tcgatttcac tgcgttgaac gattatctag |
| 5461 | atactttcaa cctttaaata aaacggttaa tttacagcag gttatgtgtg cattattaat |
| 5521 | gcagggctac atagcacgta atatttccaa acctgaatat gcacgccgat cccgacaaat |
| 5581 | ttatgaagcg ataaaagcgg gagatggtag aaatttagat gtttatgtaa atgttcccac |
| 5641 | atccacatcc gcatctggtt taacattaat tggcccatca ggcatgggta aatctactaa |
| 5701 | ttttattaat attcttaatt tatatcctca agtaattcta catccagaat acagtactta |
| 5761 | ccaaattgtt tggttaaaag tcgattgtcc tcatgctggt tctctcaaag gcttatgtac |
| 5821 | agatatattt cttggtatag ataggttact gggaaccaac tattttaaaa aatttggtgc |
| 5881 | atcacgaaat tctgaagatt atatgctggc tcaagttgcc caaatggctc atactcacca |
| 5941 | tttgggagtc ttagtgattg atgagatgca aaatttagta acagcaagaa gaagttgctc |
| 6001 | tgatatgctg aatttcttag taaaaatgga taacatcatt ggagttcctg ttatccgtat |
| 6061 | tggtaccaat gaagcttttc caattcttca aggtaatttt aggaatgcta gaaggggaac |
| 6121 | tggtgaagga agcgttattt gggacaggat gagaaatgat gatgaatggt acttttttat |
| 6181 | ggagaccatg tgggagtatc aatggacaaa aatatctact gatttttctg atgagtttaa |
| 6241 | tgatgttttt tatgatgaaa gtcaaggaat tattgatatt gccattaaac tttataaaat |
| 6301 | ggttcaatgg agagctattt cactgggtgg aaaagaggca ataaacgcca atttaattaa |
| 6361 | acaatctgtt aaagacggat tatttttagt aaaaccaatg ctaaatgcta tccgttctgg |
| 6421 | agacaaggaa tggatgatta agtataaaga tatagctcct attgatacta cagaatatta |
| 6481 | caataattgt ttatctaatt tagaaactaa agatttaaaa gaaattagta gattagcgaa |
| 6541 | aaaacagcaa gctgagaatg gacatttatc agcgacacaa cgtcatataa ttcttaaact |
| 6601 | tttagaatta gatgtagaac cgcaattagc caaagaatgt gcagtacaag taattaattc |
| 6661 | tggcgaagaa gatgctgata ttcctatttt ggtaaaacaa gcttatgctc tagctttggg |
| 6721 | tgggcaagtt caggaggcta ctgtaaagaa agtgagaacg cacacactaa aagcaaagcc |
| 6781 | aaaatatcaa ataaacgata ttcgacaaat tgcggaaaaa gcaaagaaaa atcaaactag |
| 6841 | tgcatatgaa ggtttgaaat cgacaggaat tatcaaagat ccagtccagg attttatcac |
| 6901 | tatcacctaa ttatatgctt agtttctttc caacactata tcctgatgaa ctgttatata |
| 6961 | gtgctttggc tagatatcat atacggagtg gcaataaaac ctttaagcaa actgatttag |
| 7021 | aattatttgg ttacagttct cagcaactat gtaaagttac tttaaccaat aatttgaatt |
| 7081 | atttagttaa aaacttgtct ttattttctc agttaacagt tagtgattta ctacaaaatc |
| 7141 | ataccctata ccctttttat gcaagtttct taatacccca gcaagcttgg caattacaag |
| 7201 | actctatgag gaaaaagcta ggtggctcta ttttagaggt tgcaaaagta gctaatcact |
| 7261 | ctgctgacaa ctcaacaaag tttttaaagt tttgtcctat atgtctagaa caagatacac |
| 7321 | aggaatatgg tgaaccatac tggcatcgaa ttcaccaaat acctggtata ttagtttgtg |
| 7381 | caacccatag agttgttttg catgatagct ctgtagctgt tgattctaag ggtatccatt |
| 7441 | attatgcagc gagtccagag aattgtctca ccaatcaaaa tcaagtgaaa tgcgctgata |
| 7501 | acactgtaga aaagttatta atcttagctc atgatatttg gaatataagt actattaata |
| 7561 | ttccttttaa aggtttaact tggctccgca atcaatatca gtcctattta attaatcaaa |
| 7621 | agtttatgac cctcctacct ggggagaaat ttagatttaa cgaaaaatct ttttcaaatt |
| 7681 | ctatctgtga tttttatgga caagagtttc tagaaataat taatccaaat ttaatcagga |
| 7741 | atccagacaa atatttttct ggttatttac tagcttgtga tttgaatcca gtaatcgacc |
| 7801 | ggattacaca tatattgata ataaaatttt tagctaattc tgtagaaata ttttttacat |
| 7861 | aacatacttg ttagagtact taatatataa aaatattttt tagcaaaaaa tatattaatt |
| 7921 | tacttgaaat tgcataccga ttaaagatgt agcaatagag tatggtagag aaacaacata |
| 7981 | aattaagctc agaagtagcg atttttcaga gaaatgaaat aaatgggtga taattttcac |
| 8041 | gtatcaactc agagccatgc tacagaggtt ttaaatcctc agttaatcta cgtggactta |
| 8101 | agctttcgtg taattggtca gactttaccc atagaccacg gctatgcttt gtatgctgca |
| 8161 | ctgactcact tacaggacaa acttcattct ctagatgacc tcagcattca aactattcct |
| 8221 | ggcattcctg atggcaacga ttcacttcat cttaataacc actctaggct tcggattcgg |
| 8281 | ctaccagtag ataaaatacc tttagtttat ccgtttgcag gtaaatcact aacaattggc |
| 8341 | aggcataaga ttcatttaga aattccccaa atgtatcttc ttcaagcggt ggagaaattg |
| 8401 | cgatcgcgta ttgtggttat caaaggctac gaagaaccaa agggtttttt agcagcagcg |
| 8461 | cagcgtcaac tggaaaaact gggtgtccag ggaatagcca gtatacctac caaagctgat |
| 8521 | ggcaaaccag aacgcagaag tatcaaaatt aagaaattta cagttgtggg ttttggattg |
| 8581 | gaggtaacga atttaaatga tgaagattca ataaccttgc aaaaatacgg tgttggcggg |
| 8641 | aagcaaaaaa tgggttgtgg agtttttgta ccagttaacg aaaaacgata gcagttgttt |
| 8701 | aaaggcagat gataaataca acagctaact ccaaaattca gttaaatctg agcgactcta |
| 8761 | gcactacttt gttacatcgc gcagggatag caggattttg gatgacgcta aaacagttgg |
| 8821 | aagaaatata tccgaatcct gctcaacgta ctggtaattt aacctgggta ctaacacccc |
| 8881 | atagcatcag tcttaactgg gaagggcagg atttcgcagc tttagactgg ttgctaaagc |
| 8941 | aatcttttca aattagtgat gagggtttaa ttttcctgac aggattaaat ccaaaagcaa |
| 9001 | ttcatattga aagacaaata ataatccatc aaggtattac aggaactttt ttacagcaca |
| 9061 | ataaattttt caaattttca gacaaaaaat taaagtccat aattgttgac ggcagacaat |
| 9121 | ttgtgactga gtacaaaagt gctatgtctt atgctcatca gcactttgct aaaaagttgt |
| 9181 | gtgatgaatc tggccaattg ttacaagagc ctattggcat agtaggttgg ctgtatccag |
| 9241 | gagcagtagt tcgtcattct gcttttccaa aacaaaccaa atttgaagag aagccagagt |
| 9301 | atgcactagc gttattgttt gtgcctgtag cgtgccacta ttttattttg cgatcgcaca |
| 9361 | ccaaacccca atctacaaac tatattctgg ttgtccccga agtaaataat ttagagtttt |
| 9421 | acgcccagca ttgctggaat ctgagtaatt tgagttacag agattttcat gtatctagct |
| 9481 | taagagacgc tggattaaaa ttcctgacta atgaagcagc aatacagata aaacaaaatt |
| 9541 | gtgttaagcg atgtcaggtt ttttcttttg ggacgctaat ttggtcagaa cagcaaaaag |
| 9601 | ttcgtacaga aacagcaaca ttagagctaa cggaaaaagt gagcttctac tataagttta |
| 9661 | gtcggagttg tttctccaat taccgaatcg ttgactatga aaatcaacat tttatcctta |
| 9721 | caagtctagt tcaaggaata attgctaata atttaggttt agggttgcct tggtggaatg |
| 9781 | attttggcaa catacttaca tctagtaatt tgtttaatca aatagctgat gaaaatcaag |
| 9841 | gaatttatat gatgattcaa gagtctgaat gggacataga atcgcaaaag ctgtttatca |
| 9901 | aagcttgtca tgaagcatta aaggtaatat acgccaaaat ttattcccga accaaggaaa |
| 9961 | atgaatatgc tcaaattgaa cgagaaaata cgaaaattat atctcaacta ggacgctgta |
| 10021 | ctaatgctga aaattttaga aaatttattg cggaattctg gggcaaagca gggcaacttt |
| 10081 | caattttaga agaacactgg gaagaattac taccaataac aacaggtgtt aggcattgga |
| 10141 | aaatgtccag ggatttaact tttattgcaa tcgctagcta tcccaaaagt aagaacatca |
| 10201 | agcctgaatc atcagaaata tctcagacaa gtagtgaata attagaattt tttctatttt |
| 10261 | cttaaatcca tgattttaaa tttttaggta aatactcaat gtctcatctg tttggtaata |
| 10321 | ttttaactat ttatggtact gctgctaata atcgaggtga aaatgaaggt aatattacta |
| 10381 | ccctacaaaa aataaactgg aagggcgaag ttcacaccag tgtctcagca gaggctattc |
| 10441 | gttgggcagt gcgctactac tggcaaaata atggttatga agtaaatcga cgttgggatg |
| 10501 | aaaatgcaca accactggct gatcatatct ggcaagacac gaacttcaat agtgttcggt |
| 10561 | tcattgatga tgatgtttta ggattcatgc aggcagaggc agcaagagtt gaagcaggtg |
| 10621 | agcaatctgg aggtgaagtc caggaagcat ccgcaggaga aattcaaaat gtgtctcaag |
| 10681 | atgaagagac agcaagccca aaaggcaagg gaaaaaaaac ttcgcaacgc aaaggacgag |
| 10741 | aaagatcaca agggagagtt accgctaaaa gaggtgtact agaggttact cgtgcagtat |
| 10801 | cattgattcc ctatgggggt aacattactt ttaatgcagt cagtggaaca aaaggacgaa |
| 10861 | cttctcttta tggtacagaa gttcatgcta ctcgttatca gtatggtttt gcaatgacac |
| 10921 | ccaatagtct ccaagacaaa tctcggatca atgcagttct agatggctta atttcaattg |
| 10981 | gagaagtagg aggaaatcat gcccggtttt tatatgattt ttccccagat agtattatat |
| 11041 | tacgctggac acatgatttc tctcctcggt ttctctattg ctttgaacaa gatgaatggg |
| 11101 | gaactatatc agttccagat ttagtcagac gggtagaagc tgaagatatt gacccaaaag |
| 11161 | aattatggat tggaggagca attgctagag atttaggaaa tttgggtgca aatgtgtttc |
| 11221 | caggtgtgaa gaaagtagta gaagaattat taaaggtcat tacacgagac ttacaattgc |
| 11281 | caccaaggag ttcataatgt gacgactatt gctttgaaag tagaagtccc tatcgcttgt |
| 11341 | tttcgccagt ctcgtgctag ggagtatgta gaaacctatt cggtaccacc accatcaaca |
| 11401 | gtatatggaa tgttactgtc tcttgtaggg gaagaaaaca gatatactca ctgtggagtc |
| 11461 | aagctggcga tcacacttgt atctgaacca gcaaaatcaa ctgtaattcg tacctttcat |
| 11521 | cgattgaaaa cgaagaatat tcatgatcct aaaaataata aacctgatta ccaggagtta |
| 11581 | ctgactaata ttgagtttgt agtttgggta gaccctgggg tagataaagc tagaccaaat |
| 11641 | ttagtgcaac gtttagaaga ggcatttact aacccagctt ctattaatag atttggtgga |
| 11701 | ctatgtttag gtgaaagtcg ggatttagta aatgatatta ccctcttacc aaaaaactac |
| 11761 | tatgcagaat caatgcggtg gttaatcaag gatgaacacg ggttgctaac tttaccttat |
| 11821 | tgggtagacc acgtaggttc gcaaggaacg cgatggctgc gttacgagct tcaggaatgc |
| 11881 | caagtattgc aaccgcctga attatcttgg acttcagttc aaagtgatta gggggttatt |
| 11941 | attttatttt gcaagtacct ttgcttgaga agtctaaata ccatgagcaa tagccttgtc |
| 12001 | ttcttatttg acaagccttt tagggatttt atacgcatcc aagatgcttg cgatttcctg |
| 12061 | gaacaccgtc aacacaagag ttgagggctt taacaacttt ccttgtgcta tgaatagcta |
| 12121 | ttaatttttg caactggact aatcttcagt gatagatgct tgtaaaattg aactagagct |
| 12181 | taggcgagtc aacaagtttg gcgatcgccg tgcttcaaca ttagatgcca ttaggcgttg |
| 12241 | agcaggcatc gtgcggggtg agaattgggt ttacgaaaat gtgcttcaac attagatgcc |
| 12301 | attaggcgtt gagcaggtag ttagtaataa ttaattcttt gccctttttt acttgtgctt |
| 12361 | caacattaga tgccattagg cgttgagcag agtctagata ttgcaacatt aacattctga |
| 12421 | gggttctgtg cttcaacatt agatgccatt aggcgttgag caggaagatt ttaattatcg |
| 12481 | ccatgtcggg gaaatagaag tgcttcaaca ttatcagatg ctgttaggtg ttgagcaaaa |
| 12541 | aaataggctc aagtatttgc gcttaatata aatacgttct cactgagtac aaaactagtc |
| 12601 | atcttgagtc tcgtctttcc gagaaatttt agcgataatt ttttcccatt cctcaatttg |
| 12661 | cttctgtatt tgtaatttgc gatcgctcaa ctctttatta tcattaacag acaaatcctg |
| 12721 | accactcaat cgctcacggt aaagagcaat cagttcggtt agggcttttt tattgcgtgc |
| 12781 | ttatcctcct gagtcagaga actcacattg actggaatca gctttctgcc tattctctcc |
| 12841 | acaaattcag accgacttaa atccatgctg tcggcgatcg catctaatcc atcaatcccg |
| 12901 | gtaggagtta gggctaggtt aactcgtttc ttcacctcat cgtacagttc tggtacgtct |
| 12961 | ttttgacctt tttgcccttt cttacccatt gccgtctact ttttacattc ctcccaatca |
| 13021 | tagtaaacac attcaacact ttaagctaaa cattaaaact gtttctaatc tttaggtatt |
| 13081 | gtgtaatgcc taatttagct agataattta ggtattgtgt aatgcctgaa taggcatctt |
| 13141 | gtaggaatat gagtaactaa tttcaccgtt tggcttatca acatctaaac ggcttaattt |
| 13201 | tcatgggcaa tccttaattg ctgcttatct aattcaaatt aataaaaatc tgctaaataa |
| 13261 | ttcggtcatt tttcacaact atatggtgag ttttttccct aaaccttatc cagaggaaat |
| 13321 | tttatatagt gtcattgctc gttatcacat tagaagtggc aataccagtc caaaaattac |
| 13381 | acttcaagag ttatttaatt cacaaactac tgtagcaaca gtagatttac catcgaacct |
| 13441 | gaataattta attcaaaatc tacaatttat ttcaaattac caagttgagg atttgatata |
| 13501 | taaacatact ctctaccctt tatatagccc tttcttgtct gcaaacaggt ctagtcaggt |
| 13561 | tttggaatct atgaaaggag attacggagg agatattcat acaagagccg gaattatggc |
| 13621 | aagctcaatt actatgccaa agtattttcg gttttgtcca acttgcctag aagaagacct |
| 13681 | aaaaacttat ggcgaacctt actggcatcg catccatcaa ataccagggg ttttagtttg |
| 13741 | tccatttcat gctgaagttt tgcaaaatag tctagttccc ttacacggtt ttaataagca |
| 13801 | tgaatatcat gcagctagta cagacaactg ttctattact aaagaatcaa ctttttttag |
| 13861 | cactgacata ctgaaaaaac tactgttatt agccaaagac ttttggtggt tactcaatag |
| 13921 | tgaaatacct tgccaagaac cacagtggtt tcgcaaacaa tataccaatc tgatgattga |
| 13981 | gcagcgtctt gcaactgcaa caggtcgaat ttatcagaaa agactactgg ataatttttt |
| 14041 | gtttttttat ggtcgagaaa tgctcagtgc cgttgattca atggtaaatc ataatgactc |
| 14101 | aaacaattgg gttactagca ttgtcaggaa gcataggaaa tctttccatc ctcttaggca |
| 14161 | tttactgata atcagttttt tgaaaacgtc agtagaggaa tttttcaagc aagatactga |
| 14221 | atacaagcct tttggcgaat ctccttggtt gtgtctcaat gctgccgcag aacattatct |
| 14281 | acaaccagtg attaccagtt tagacattac tttatgctgc gatacaaaaa agcctgtcgg |
| 14341 | tactttttct tgttcttgtg gaatgattta ttgccggact gggcctgatg aaacagatga |
| 14401 | agacaaactt cgcattggta aaattaaaac atttggtcaa ctatgggagc aaaagctgaa |
| 14461 | agagttagta gaagttgaaa agttgggttt gcgggagaca gccagaagat taaatgttga |
| 14521 | tgctagaact atcaaacgtt atgtatctcg tctgagactg acaacttatt ggcagactag |
| 14581 | aaaggaaaat gactctgtag gcttacagga attaccagaa atagatccca attttgcaac |
| 14641 | tgaactcaaa ttgaaacaca ggcaaaattg gatggtttta caagtacaga accctgaagc |
| 14701 | ttcaaagaca actctgagaa aactagcacc agccacctat atttggctat acaggaatga |
| 14761 | cagagaatgg ctcaatcaaa actcacccgc tttacaagtt gctgttcctt ccgttgtgag |
| 14821 | agttgattgg catgagcgag atcagcaaat tctcactcag gttcaagatg cagtgcgatc |
| 14881 | gctacttaac gctgagatac cagagaaaat ttctattagt agggtaggga aaactatcgg |
| 14941 | cttacttgca ttactcgaaa aacatctcga ccaaatgcct ctaacaaagg cttacttaga |
| 15001 | atcagtagtt gaaacggtag aagaatttca aatgcgccgg atcaagtggg caatcaagtt |
| 15061 | gcttgatgaa tgtggagaag aaattatgcg ctggaaggta gtcagagttg ctgctttgcg |
| 15121 | agaggattgc tctgaaaggg tcagtgcatt tctggaaagt gaacttgaga aagcatacaa |
| 15181 | gaaagagagg tgattcaatt cacctctcca agtgatggga taaatgcttt gtgtgttagg |
| 15241 | ccgcgtcggc taccagtaag ccattcatag cgatgatgat gatttgtgct gctaaaggag |
| 15301 | tatcacaacg tggtctatcg tcgctattac agggttgtgc tacccattta ccctccagat |
| 15361 | tttggtagaa agtgccgaga agttgataac cattccaaac acggtaaagc tccccaaata |
| 15421 | cttcagatac cgaatcaatc tcaatttctg gtgctactgg gtcagcaagc tggtcaatat |
| 15481 | attcctcaaa ctctaattga gctaaggctt ggttatcaaa ttcatcttgt acaatcatca |
| 15541 | tgagtaacct cattaatggg gttgaacaaa aagcgatcgc atttctcttg gcggagggcg |
| 15601 | atcgcctttt tgatatcatc ccacaatatg gctactttag tagctaaata ataacaaatg |
| 15661 | tatctaaagc tttaatttgt cacttcgact gtgcttaaaa aattgcttac cgatatttcc |
| 15721 | aatgcagtac atatagcttc taatactttt agggaaacac tttcagactc accatattct |
| 15781 | aattttttca agttttgatg ggaacattca ccacctctag cagcaatttc atcagctagt |
| 15841 | ttctgacgtg attttttacc acgcaatcgc tttaaaatga ttcctctgtc ttgattccag |
| 15901 | acgacagata caacatctgc aacataaata tacgtttttg tttctttttc acccttattg |
| 15961 | cccatatagt agctacaaca gtaaacaata cataaatttt gagctactat agtttacaaa |
| 16021 | ttattaggat ttaggtatct caaaacgaag ttgcgcccgg tcttcaggat gagagcgtta |
| 16081 | tcatccaaga agatacacaa tgcaggtttt gctaagagaa tgtgtcatag ccaatattat |
| 16141 | gagtaatcgc ggattcctta aagaggtagc aaacaaatat atattaggct gattatgtta |
| 16201 | atttgctaaa aatgttcagt tcttaaaaat aactgaacgt ttttatctaa gtataagtac |
| 16261 | gagcttattt tattagtaga ttattaaatt aataaattaa taaattaaat aattacttgg |
| 16321 | gctatgacac cgtataaaca cggttttaaa atcaaaataa ggatttaaag aagaaatatc |
| 16381 | tataaaagta tttgcggcaa aatgtggcgg ttaagagtat atgcaaatac aataacttga |
| 16441 | atatgttttc ctactattat ttgtcataag tgtatccttt atggcgcaaa ccatagtaat |
| 16501 | acccacgggc attcctatca taacccttcg cctccaggta cttgtattgg ctttggggga |
| 16561 | agtttcaaca tggtggcgaa ctcaatattt ttctgctgct ggactgagat ttttagaaag |
| 16621 | aatctaccca cgtacagctt ttgccgcagc tatcaatgct gctagtacgg cagctcgtcc |
| 16681 | tgttcacgac gctagtactg gcagtaacca aatctaccat ctgtttcgcc ttcctatttc |
| 16741 | catagagcgg gaagttcaac tattactaac aaaggcacaa ggagcaagtt tggcgttaga |
| 16801 | agtcaaacca gcacttgggg ataaagacaa gttgatgaca atgttagatg cactaactac |
| 16861 | agaaaaatct aaggtagcgg ttggtgcatt acgtttagga agcagtgcag aggcatacca |
| 16921 | actagcaacc tacaaacgga tggcaggagc atatctaaga ggttttcaaa caggcgatcg |
| 16981 | cgtctttccc tacatggagg cagaccagga gtgattgtta tgacatcggg tgacgcagtg |
| 17041 | caaaagtcac ctcgttacag tattgcgatt gtctgtaagg gaactgcaat ggtgaacgag |
| 17101 | actcgccatt taattcaaca gtggcatcct acagaaaatg tggatacatt tgttaaacgc |
| 17161 | gttcaagctg aggatctgct gggacgacaa acagcccagc gagcaaagga cattgcccga |
| 17221 | aaagtctttg cagcacgctt tttgctacca aacaacaaac ctgctcgact ccttaaacaa |
| 17281 | attgttgagg ctggtcttcc ccataaaact tttgtagaaa tactattttt atttgcgtgt |
| 17341 | cgtgctgata atttattgta tgattttacg ctaaatattt actgggcagc agtagcacaa |
| 17401 | agacgaaaga aaattttcac tcaagacgtt ttagacttct tcgagtcagc tgctgttgat |
| 17461 | ggacgaattc tctctccttg gtctgaggga acgcaggtgc ggacagctag gggattgtta |
| 17521 | gcgacgttgc gtgatgttgg ttttttacgc gatgttagtc atggtaaatc ggagcgagag |
| 17581 | attattccct actacatatc tgatgaaggt gtagcttgcc ttgcacgcta tctccatgaa |
| 17641 | tcaggaattt ctaactcagc tttgtgtgaa caccaagact ggaatatttt tggtctcaac |
| 17701 | cgtgaacaag tgtgcgatcg cctagatcaa cttggagaac accgtggatt aatcatacaa |
| 17761 | aaagctgggt cagtagtcag catcacttgg aaggtcaact ctatggagga acttattaat |
| 17821 | gtcctctctc gatgaaactt ttgaattact tttacaaaag ctagaccgcc cagacaccct |
| 17881 | caatgctgct aaaagtgacc caatttttta cttcgtttac aaacccgaag atatgctcac |
| 17941 | cctaaagcag cgtttatcgc tttggaaagc taaattaagg gagttgaaat ttgatgttga |
| 18001 | aagaatatcg ctctccgacc ttttgtggca gattattgac gaatcagggc ggtgggaaga |
| 18061 | ctggctagaa attgaacctt cagcagaggt tgaggagtta aatcaagcca ttcgtgatgt |
| 18121 | tttaactagt cagagtcgtc taattgaaga ggtggcacaa cgaattaatt ctttgatgcc |
| 18181 | tcccaaaatt gtttttttaa ctgaagtcga gatgctgcat ccttatctac gctctcggac |
| 18241 | tctagaaaac aagcttcatg atcgcgtgcc agtaccattg ataatttttt atccaggtta |
| 18301 | tcgggcggga cagtacggtc tacgcttttt aggtttccac gcagaagaca gtaactatcg |
| 18361 | ctcgataatc gtcggaggca tttgatggca acaattcaat ctcttttttc cagcagtaga |
| 18421 | cctattgacc gacagattga aaaggtcatc gattattctg cacaggatga agacagacta |
| 18481 | gctacagaaa tttcagaata tgagattacc gataatattg agcgatgtta tcggaggttt |
| 18541 | ctggatgtat tttcggaagg agttcgtggt ggacgcgtaa ctgagactgg aatatgggtg |
| 18601 | tctgggttct atggttctgg caagagttct tttacaaaat atttgggatt tgcattagaa |
| 18661 | ggagagagaa aaaaagtacg tggcaagcca tttctggaac ttttatctga gcgctttgtt |
| 18721 | aatcgcaata ctatccaagc cgaactacta actctagtta gacaacattc cactgttgtc |
| 18781 | attttccttg atttaggttc tgaacaactt tctgaaagtg ctgccacgac tgtttccaca |
| 18841 | gtcctttatt ggaaagtatt gcagtgggca ggtttttcta aagaaaagaa actagcacaa |
| 18901 | ctggagttaa ctttagaacg tcaaggaaaa tatgctgaat ttcagcagct atatcgagat |
| 18961 | acgtataaca aagaatggga agaaattcac aatgatccct tactaggggt tagtcgcgct |
| 19021 | gcggaaattg tcactcaagt attacctaaa gaatttccta caagtaaaaa ctttcgagaa |
| 19081 | ttgagatttg atctggctga taatttgcgc gatcgcacca gtgaaattat tgatttagtg |
| 19141 | cgacagcgaa ccaaatgtga aaatatctta tttttactcg atgaagcggg tcaatacgtt |
| 19201 | gctcctcgtg gggaattgat tcttaactta gatggaatgg ctcgtaattt caaagagtta |
| 19261 | ggtgaaggaa aagtctggat aattgccaca ggtcagcaaa ccctgaatga aattgtcgaa |
| 19321 | aaagctgcct ataactctgc tgaactgaac aagctgaaag acagattccc aatctctatt |
| 19381 | caccttgatg ctagcgacat tagagaaatt acttatcggc gattattgac aaagccgcct |
| 19441 | gataaagaac aattattaaa agatttattt gctacccacg gtcaagcggt tttaacacac |
| 19501 | acacgactaa ctgaaacagt actttataag aatgatccga acgctaccac ctttgctcaa |
| 19561 | ctttatccct tcctgcccca gcactttgat ttgctgctgg agttgattcg caccctggct |
| 19621 | cggtcaacag ggggtgttgg cttacgttca gccattcgtg tgattcaaga tgttctcatt |
| 19681 | gacaaaagcc gtgttttgcc agcaaacact cttaaattag cagaacgtcc tataggtgtt |
| 19741 | ttggcttgtg tggatgactt ctacaacact ttgcgcgaag acatcaacaa agtatatcct |
| 19801 | cacgttgtga gtggggcaga taaagttgtc agaatttttc ctaatgatga gatcgcccat |
| 19861 | agggtagcta aagctgtagc tgctttacag ccgattgata cctttccgcg cacagcagaa |
| 19921 | aatattgctg ctcttcttta cccagaattg ggttcgcttt ctttattcga tgaggttcaa |
| 19981 | caagcactca agagaatttt aaatgagaag gagtgcggtt taattgaaga ccctcaagca |
| 20041 | ggtggttatg tttttctcag tgaatctgtt agacctttgc gagataagcg aacttcctac |
| 20101 | atccctacgt ctaacgaatg ctctcgcatt aaagaagata ttgttaagca aatttttcgg |
| 20161 | gatcaaccat ccacaagact tgacaatatt aaggaagtaa aagccattgt caaactagag |
| 20221 | cataagctag tcgttggtcg ggatgaaacc atcgatttca aattagaatt cgttgatgct |
| 20281 | ggcggattct ccaatcgccg tactgaattg atggcagaaa caaacagtcg gcgagaattg |
| 20341 | aaaaactcta ttgtttggtt ggcaaaatca gacaacacag tagatgaact actgcaagaa |
| 20401 | attgctcgtt ctgagtgggt agttagagaa attgatgagc gagcagatga taaaggagat |
| 20461 | gtggcacagt ttttgcgagc agagcggcgt tcaggcgatc gcaaccgaga cgaagtagcg |
| 20521 | aaaaaaattg agcgcttgct gatggaagga actttgattt tccgaggaaa acctacacca |
| 20581 | gttagtgagg caggacagac tctaaacgct ggagcgcaga cggttctcgc aaatgcagca |
| 20641 | aaagaagtgt ttcatctaca caatctcgca gctattcgac cagcaacgga tgttgctgct |
| 20701 | aagtttttgg gcgtagagcg tttagatcgc attacagagc aacttgatcc tttgcgttta |
| 20761 | gtgactaaaa aagctagcaa tactcgtgtt aatgtcaacc attcagcttt ggcggaagtg |
| 20821 | ctgcgagtgt tccgggcaaa agtagatgag tctggttcag gtcgcttgca aggaaatttc |
| 20881 | ctacaagatt ttttctcggc agatccttat ggctggacta aggatacagt ccgttactta |
| 20941 | tttgctgcac tattgacagc tggtgaaatt gaattacatt accccaaagc tgatggccca |
| 21001 | ctgcggacac cggggccttt agccgtggag gcaatgaaaa atactatgga atttagccgg |
| 21061 | gtgggagttt cattgcggga tagcaaacct tccattgaag ctttggagcg ggctgcacag |
| 21121 | cgtttagaag agctttttgc tgtggaagtt gtaccgttgg aagacaatat cagccgtgca |
| 21181 | gtacaaaaac agatgccaga gttattagaa caaattgcag acttacctgt tcgtctacgt |
| 21241 | cttttggatc tgcggggaga acaaaaggca aaagaactgc aagcaagttt aacggatatc |
| 21301 | ctcagaggcg atgctagcaa tgcaacagca cttttaggta gtcaaaattg cacaattcct |
| 21361 | gacgatatcc aatgggcaaa gcaaattgtt aatttactca acaatggcgc tgaaagagaa |
| 21421 | ttcaaagtag cacgatcgtt actcaataac ttagatgaac ttaaggattt attttccaag |
| 21481 | caggtgctag aactgtcatc tcaaggtaat cgagaagcta tttacaatgt ttttcactca |
| 21541 | gaacggtttc aagaacatct accagagttg cggcgagcag ttcgagaatt gacggggcaa |
| 21601 | ttagaagaac gctacacaac tgaggttgca gagtataaac aagacttaca aaaagcctta |
| 21661 | aacggcttgg agctagagcc agaatggtta aaactaactg acgagcagcg aaataatatt |
| 21721 | gccgatcgct tgcagatgga tgttgccgaa caaattgacg gacagaatcc agtgcgatcg |
| 21781 | ctccaggtga ttctcacccg ccgtttaggt ctaactggag tagttgataa tttacggcgg |
| 21841 | cagatacaac aatcagtgcc acatgaggcg gaatcaacaa atgggctagc aggtacttac |
| 21901 | tctgtagaaa atctacatga aacagaaaga gtgaatacac ctactggtga atacactgta |
| 21961 | gaaacagcaa ttgatgcagc cgaatttgtc atgccagtac ttatagaaaa ttctgagcaa |
| 22021 | ctggatactt ggttagcttc cataagagga aaactactag aaattttgaa tttacgtaag |
| 22081 | cgtatacgca ttaagggata aatatgagct ttgatcgaga aaccctcaac gccttagcta |
| 22141 | gaatggttgc agcgtgtagg cggcgactga cggaagatgt agaaaatcaa ctccagcaga |
| 22201 | ctttcgggtt gtatgtagat ggcacaattc ttcccttatc ccaattgacc catctgacag |
| 22261 | aggagcaaaa ggttgcagcg caagcattac gtgaactgct aaaccacttc aaagccagcg |
| 22321 | acccacaaaa aagctatcaa tctgcttaca atcgcacaat tctggaaatc tcctttacca |
| 22381 | tcctcaaccg tttggcagct ttgcgcttgt gtgaggagcg gaatttagta attgaatgcg |
| 22441 | tccgcaaagg aatggagtct gacgggtttc gcttgtttga aagacttgcc aatggcagtt |
| 22501 | tggggagtcg ttacgaaacc tatcgggtgt ttgtggagtc catgtttgac gagttggcgg |
| 22561 | tggatttggg agtactattt gaccgcacta ctccacagtc aacgattttt ccgagcgatc |
| 22621 | gcgtcttaac agaagtcttc gatttgctca atgatgtcac actgcaactt gtctggactg |
| 22681 | aagatgaaac aataggctgg atttaccaat acttcaatcc acctgaagaa cggcgagcaa |
| 22741 | tgcgggaagc gtcccaagca ccccgcaata gtcgggaatt agctgttcgc aaccagtttt |
| 22801 | ttactccacg ttatgttgtt gagtttctta cagaaaatac ccttggtcgc ctctggtatg |
| 22861 | agatgcgtca aggagaaact cgaatcaaag atgaatgcca gtattttgtc gaaaattctg |
| 22921 | agcaattctt agccagggat cgtaaagacc cccgtgatat caaagggcta gatccggcaa |
| 22981 | gcggtagcgg tcattttctc ctttacgctt atgacttatt tgaggtgatt tatcaggaag |
| 23041 | catgggaaga tgaaaattcc ccgccctcag aatttacggg tcgaaagttg cgagaagatt |
| 23101 | acccaacttt agaagagttg cacagacagt tacctgtatt aattcttaag tacaatttgc |
| 23161 | atggtattga cattgatgcc cgtgcttgtc aaattgccgc tcttgctttg tggctacgcg |
| 23221 | ctcaacgtta ttaccaatcg ctaaatatta aactaagtga acgtccaccc atacaaaaaa |
| 23281 | ttaatattat ctgcgctgaa cccatgcctg gggaagaaga attactcgca gaatttatcg |
| 23341 | atgagttgaa tcagaaacgt ccaccaatta ttggtgaatt ggtaagaaaa gtttttgaaa |
| 23401 | agatgaattt agccggagaa actggagcat tacttaaaat tgaagaagaa attagagatg |
| 23461 | agattacaca agctaaaaca caatggctag ctgaatataa cccagagcaa ccaactctat |
| 23521 | tttcattagg agaatattca gatgttaacc aattaagttt atttaagttc gccggaatta |
| 23581 | ccgatgagca gttctggact caagttgaag actgggttat agaagagctt cagaactatg |
| 23641 | ctaatcgagc aacgacaggg caagttttcc agcgtcagct attttctgac gatgcgctac |
| 23701 | agggatttgc ctttgtggat atctgccgta agcagttcga ttttatatta atgaatcccc |
| 23761 | catttggtga ggtaagtatt ccttcaaaag aatatattga agaagtttat gaagatacca |
| 23821 | agggtaatgt ttatcaaacc tttgttgaat gtttccaaga caggctagta gcaggaggat |
| 23881 | tcttaggggt tatttctagc agaacaggct ttttcttggc gcaatcaagc gattggcgta |
| 23941 | aaagaatatt actgcgtttg tatcgtccgt tattgttagc cgatttaggt tatggtgttt |
| 24001 | tggaagcgat ggtggaaaca gcagcctatg ttctacgtag tattacaata gaagaagata |
| 24061 | aaaacttaac attaaatctt gttccacaac tattagaaat tcctgtagat agacagagat |
| 24121 | gttttagcat tcccaaatat cagaaacatc gaggcggctt aaagcggcat caggctaatc |
| 24181 | aagaattaaa aaaactgcac gaaaatggtt atatatcatt gacactaggt cgttttattc |
| 24241 | gctataaagc taattttgca aaaattcaac agtctgatat tcctagttat ttagcatatc |
| 24301 | ctaatttaat ttgttttcga ctgttggagg aaggagataa aaaaagtgcc ttaatagata |
| 24361 | ttttacaaaa ttacaatgac tctagatatt ttgtagtttc gccagaaagt ttttcaatgg |
| 24421 | ttcctaatac accattttgt tattgggtaa gtaataatat tcgccagtta tttactaaac |
| 24481 | tgccgcaatt tgaaagtaat ggtagaatcg ctcaacacgg agcatcaaca aaaaaagatc |
| 24541 | cacgatttct acgtctttgg tgggaaatac tacctaataa aatagtaact ggtacggtag |
| 24601 | aaacaacacc agaacagttt tgtcaacaaa cttttgagga taagccttgg gtcttatttg |
| 24661 | ctaaaggtgg agcttattct ccatattatg cagacttaca tttgctaata aattggaaga |
| 24721 | ataatggcga ggaaattgaa caatatgttt tatacaaata tccatatcta ggcagaagtg |
| 24781 | gtgcagattg gattctacat agggagtgta attatttcca agcaggctta acatggacta |
| 24841 | ctcggactac tagcaagatt agttttcgga atttgcctgc gggtagcata tttagtagta |
| 24901 | aaggagcagc agcttttact aaccatgaaa tgtattggtt ggctatttta caatcgtcta |
| 24961 | tctctgaagc tttaattagt ttgcagcttg cagcagtaga tgcagcagcc cgctcttatg |
| 25021 | aagtaggttt aattcaacgt acacctgttc ctcctaatag ttctgatttc cataaggaaa |
| 25081 | tattagtaaa attagcagat ttttgtattg gtattaaacg ctctttagat acagtaaatg |
| 25141 | aacatagcca catatttcat atacctgctc tgcttcaagc attaggtaat aacttaactg |
| 25201 | ataagatttt taattgggaa gataatatta ataaatccac taaacgatta agtaagtatc |
| 25261 | aagaacaaat tgatgatgtc gtcttccaac tttacggaat atcaaaaaca gagaagaaaa |
| 25321 | gtcttgaaat aaatattaaa aacaaaaata taagaattga agataatact gaagatatag |
| 25381 | acgaagaaga tgaaactgca ctagcagcag acgcaaaaca gctagttact gatttacttt |
| 25441 | cttatacact cggttgctct tttggtcgtt gggatatccg cttttctaca ggcgataaat |
| 25501 | cagcaccaga acttcccgac ccctttgcac cattaccagc ctgttcaccg ggaatgctta |
| 25561 | ttggtactga tggtttacca gtttcagaaa ctccgccagg atatcccatt cccattgctt |
| 25621 | gggatggcat tttagtagat gaccccaatc accccaacga catcacccgt cgtgtccgtg |
| 25681 | aagttttaga aattatatgg aagaacaacg ccaataaaat tgaacaggaa gcttgcgaaa |
| 25741 | ttcttagtgt gtgtgaactg cgggattact tccaacgtcc tacaaacttc tttcaaaaac |
| 25801 | atctcaaatg ctataccaaa agtaaacgca aagcaccaat ttactggtca ctttctacaa |
| 25861 | catctagttc ttacacagtc tggatttatt accaccgcct tacagaccaa acgctctaca |
| 25921 | caatcgtcaa ccgctacctt gaaccaaaaa ttgccgaggt tgagcgtatc actggtggct |
| 25981 | tagaaaaaga attagagaca aaatcgggac gggaagcttc acaagtacgc gataacttac |
| 26041 | acgcaacacg caaattcctc agtgaattgc aagatatgaa gcaggaactt ttgcgcgtcg |
| 26101 | cacagctacc ttacaaaccc aatctcaatg atggcgcaat tatcactgcc gcccctctac |
| 26161 | accgtttatt tgctttgcga cagtgggcgc aagaaaccaa gaaatgttgg gaaagtttgg |
| 26221 | aaaaaggcga atacgattgg gcgcatttag cctacacaat ttggacagac agagtgcggg |
| 26281 | aagtctgcaa aagggataaa tcaattgcga tcgcccacgg acttgaagaa gtttaccaag |
| 26341 | cagctaaccc agaaaattcc caacccaaaa caaccaaagg cagaagccgt aaaaagacga |
| 26401 | cacaggagtc agtataatga gcgtctcaga ctttgtttgc cgacaattac accaaagatt |
| 26461 | agaacagcac cgtattgtcg tttggtacga tggcaatcgc gcttttgctg attttattca |
| 26521 | atccttccaa gctgactcct gtgtagtcgt atctgctgtt gagtctattt tgaactcccg |
| 26581 | tcgtcaagcc gaagccgtct accgccagat gaacgagtct aataactcag ccaaagctgg |
| 26641 | gttgaatctc ctcatttacg taccccgcca gcgcggaaca atagaagatg agaagcggca |
| 26701 | agatccgttt gaagttttcg catgggctgg tgtggcgttt ggcgacaaag aagcagaaca |
| 26761 | gttagaatct cttgctagac aagcaatgcc agaacgggct atagaaatta ctcggctatt |
| 26821 | tgcccaaggc agtccaacgc tatcgatgtt ggataaatta cagttccatc accgttcgcc |
| 26881 | tttactgtta gaggcgctag gtactgactc accatcagaa gcgatagcct tagtcctctg |
| 26941 | cttagaggag aaatcacaac gaattgattc tgttaaaggt tgtttaaaag aatttttgcc |
| 27001 | gttattggca acggagattg gttttaaacc tgcatccaaa gctaaatcat ggacagaaat |
| 27061 | tagaaagcaa ctcattcagt atgtgctgtt tagcgaattt gccctcgacc tgccttgtga |
| 27121 | cctgccagag tcattaagta ccttacccag agccaaagcc agccatgaat tagtgatttt |
| 27181 | tgcagtgtgc gatcgcatga gggatagttc cgacttgcga tcgggctatg ttcagctagc |
| 27241 | agtagaagta gaaaaccagt tgcaccttgg caatctcaca gaaaatatag ctcaactagg |
| 27301 | tactagagat accttcccct ttgcagaaaa gcgataccta cagcaagtgc tggagtatgt |
| 27361 | gcaacagaat aacttagcag atgcccagaa gattttaacc caacgccgtc attccatctg |
| 27421 | gaacaaccac ttggaacgag gtaatatttg gatgatagta gagcgttgtg tgacatttct |
| 27481 | cgccacaagt aatgctgtag aagagttgtc aaaaaaatct ttacagtcaa tgtctttacg |
| 27541 | ccaaatcatc tctgcatacg cacctccaga tcgctgggga tgggcagaac tagaccgaca |
| 27601 | tcagcgacta tttgaacagg gtgcaaccgc ctatgcagga gatgagggag agatagaatc |
| 27661 | tttagtcgaa ttttgccgcc gacgttactt agaagtggca ttagcaattc aagactgctt |
| 27721 | ccttaaatgt gttcagaaag aaggttggtt tcccgaagga gtgctgcgat aaacccaaat |
| 27781 | atttgaccag tatgtagctc ctctactgga acagaacaac aaaaaagtcg cattttttct |
| 27841 | agttgattcc ctgcgcttcg agatgggacg agatttggca gaagctttag aacagttggg |
| 27901 | cgaagtcgag attttaccag tcgcatctgt tttacctaca actacacctt gcgggatggc |
| 27961 | agcactgatg cctaatgctg atggtacgct cagactggtg gaatcaggcg gagagctaat |
| 28021 | tcctgtctta ggtgaaagga aactggaaga gtccaagcac cggatgcgac tgctacagga |
| 28081 | aaaatacggc gatcgcttca aagatatcga accggaagat attttcagca aaaaggtaaa |
| 28141 | tgccttgggt aagcagttag cctctgtgga tttattagtt gtccgcacca aagaccctga |
| 28201 | ccgcattgct gaaagtattg gcaacatgaa agcccgaaaa tatctttcgg atatagtcgc |
| 28261 | tgaaatggta aatacaattc gtcgattatc tacaattggg ttcacccatt ttgtcattag |
| 28321 | tgcagatcat ggtcacgtcc taattgacga aataccatta ggagatgtaa ttcccaaacc |
| 28381 | agggggtgag tggttgaaaa ctaagcgcag gagtttgctt gggcaattta tatcgggcga |
| 28441 | atcttcaagt gtagttatct tcaaaactga gcaggttggt atccaaggcg atgcccaaga |
| 28501 | cctctgtgtt gctaaaggct ttaaagtttt ccaacaagtt gaaggttact tccacgaagg |
| 28561 | cataagcttg caggaagcga tcgtgccatt ggtaattttg cgatcgcaag gtaagattac |
| 28621 | tgctcagagt aagccaaata ttttcctgcg ttataagtac gattacttta ccaataaagt |
| 28681 | cattaatctg cgagtctgct atgaatattt gctaccagaa gcaatacaag tccgcattga |
| 28741 | agcttacgat ggctcaacag caaaagccaa gcgggtaggt aaagcaaccg actgtgatgc |
| 28801 | tagagacgaa cgaactggtg aaatcatact ccagcccaat caagaaactt cggttcctat |
| 28861 | cttactggag tctgacgaca tttcatgctc caaaattgaa atacgagtaa ttgacccaca |
| 28921 | aactggcgta atttgggcaa agttagaact ggaaaatcgc ttactagatt aagaatgaac |
| 28981 | tcatgtttca agatgcctta gaccaaaaag ttaatcaaac atttccagga aaggtagtac |
| 29041 | gtaaggactt gctacatcaa atcaagggtg gagaaaatgt accatcttat gtactggaat |
| 29101 | atcttctggg aaagtactgc gcctcagatg atgaagatga aatccgcatt ggtattcagg |
| 29161 | ctgtcaaaga aacactacaa agcaactact tccgccacga tgaagctaac aaagcccaag |
| 29221 | cgctagtaga acaacgggga cgacatcgat ttatagaccg tatagaagtt cgttatctcg |
| 29281 | ccagtgaaaa caaatattgg gcggcaatgg atcactttag ttactccaga attcatgttg |
| 29341 | ctgaccgatt ttatcgacaa tacgaacgac ttttagaagg tggaatttgg ggtcttgtag |
| 29401 | atgttgaatt tcaaccaact gaagaagaag gtgctaaagc cagtcccttt cacattgctg |
| 29461 | acttaagacc aattcagtta gcaagattta acgttgatga atatgcagaa ggtcgtcgca |
| 29521 | gcttcagccg cgatcaatgg attgatattt tgctgcggag tgtgggttta gaacctaccc |
| 29581 | aaatagaaca acgtttaaaa ctgctgttac tgactcggtt cattcccttt gtcgaaaaaa |
| 29641 | attataactt cattgagcta gggccacgag gaacaggtaa atcctatgcc tacagtgaga |
| 29701 | tgtcgcctta cagcatcctc atttctggtg gtaaagccag cactgcaaac cttttctaca |
| 29761 | acaatgcccg ccgtcaggta ggtttagtag gtcattggga tgttgttgct tttgatgaag |
| 29821 | tcggtggttt gaaagttacc gatgcggatg caattcagat tatgaaagat tacatggcta |
| 29881 | acggtcgttt cagtcgtgga atcacccaag ttttagctga cgcgtctttg gtatttgttg |
| 29941 | gtaatcttaa ccaacctgtt gaaactctag tccaaaatac tgcaacagac ttatttcagc |
| 30001 | cattaccaaa agaatttgat ctggctttac ttgaccggat gcacttctac ttgccaggat |
| 30061 | gggaagtgcc aaaaaactcc aaaaatctgc ttacagaaca ctatggtttt gtcactgatt |
| 30121 | atttggctga agcgtttcgt accctaagaa aacaaaatcg gtttgatgaa gtcgagaaag |
| 30181 | tcttccgttt tggttctcat gtagaaggac gagatgcgat cgctgtcaaa aaaaccgtta |
| 30241 | gcggattact taagattatc catccagacg gagaatggac aaaggaaaac ctgacagaat |
| 30301 | atgtagaact agccttagaa gggcgcagac gagttaaaga acaactcaaa aagcgaggtt |
| 30361 | ctttcgagtt ttataaaacc agtttttctt acatcgacca ggaaaccgaa gcagagatga |
| 30421 | cagtgggtgt accagaacaa ggtagtgctg gaattatctc tcaagaaccc ttacccccag |
| 30481 | gcacagtgta caccgccaca gcagatggag aagcaagggt tggtcttttc cgcttggaag |
| 30541 | tagcttgtac atcggggaca ggaaaattgc gaacccctac aggacttgac aaagctttga |
| 30601 | aagaatcatt aaaccgcgct tacagctatc ttcaaagtat aaaggaaaga aaaggactca |
| 30661 | atgaaacttt gggattgaaa gatatttacg ctgaagctgt ggacttgtct ggaagtcgtg |
| 30721 | ttgaatgtcc ttgtggtgta gcattttttg tcgccatcat ttcagccata cagaatcgtc |
| 30781 | aaatccaagc tggaactctt atcttgggcg atttaacgat tcaagggaat atcaaaggcc |
| 30841 | ctgcttcaat tgttgaatca ttacaaatag caatggaaag tggagcttta cgagctttag |
| 30901 | taccactttc taataaaagc cagtttgcag gtttaccaga agagatagtc gaaaagctgg |
| 30961 | atattgtgtt ttacggtgat gttgaccgag cagtaattaa gagcgtcgaa atttaagaga |
| 31021 | ctatccctat tatgtcactc tcgaaaaaca ataattgaaa caagattcta aacctttgat |
| 31081 | ttaattaagc ttcagccttt attttctaac aataactaaa tttatagcca aaatcagaaa |
| 31141 | attaaagccc tgaaagcttt ccagcgattc ggttctccta aaatgacaca acagggctat |
| 31201 | ttataaaata aatctttagg ctacaagacg acttagcata atcctacttt gagaaacctt |
| 31261 | taatgtagct gatgagatgt tgatctatat tgtcgatatc acctttaagc tactgaaaat |
| 31321 | cgagttctga acggttatga aatgcgattg ctcgtgacct tgtagatgca gttcaccgca |
| 31381 | attttgctat cgactggcca cagaaataac tttttaaaac taacctctgc cgattagcgt |
| 31441 | gtgaggtcga gaattttgtt gtgcgatcgc ctcactacga acaaatgcta acttttttac |
| 31501 | acccctttaa atttgcttgt ttctagtatc gctgtttcta cgattttttt gagattaata |
| 31561 | gcacctccaa ttacagtaga aatactgtga atttagcttt tattgacttg gtacttagct |
| 31621 | aaaatactat tcttctattg gagtattgag gagcgctcgc gcaataggaa aagcaagaca |
| 31681 | caatatttat ttataaaaag gaaataattc agcttaaagg tttcttaatg gaacctgacg |
| 31741 | ggaactttca atctttcatc aacgtgatta tagtaagctc aacagtaaat atacgattta |
| 31801 | tagttattta gttattgtta accttcaagc catctaagct aaattacgga tgattaaccc |
| 31861 | ccagctagac aaaactattg agcgttatga gacggaaatt gagagactga aagtgactga |
| 31921 | cacggccata gatgccgaac aagttttgga agtgttaagt gccagggatg ctgtgcagtt |
| 31981 | agccttaaag ggtgcaacat tcattcctag cagtaggcta aagcaggttc tagagttaga |
| 32041 | tgcagaactc aggaaacaag cagtacacat taacaaagtt atcaaagctg aacaattcgc |
| 32101 | ccagtggcga gaaagtacac atcctcctgc tgaagcttgg tggtggcggt tagaaaatct |
| 32161 | tacccctcac cataagtggg atagatttga ttggttgtgg aagggtctag tagttgctgg |
| 32221 | ctggactgct aacctcagtt tattaatcaa tattgctact cgctttttga gcgggggagc |
| 32281 | aggattattg ggagcttctg cggttatttt gccgagtatt ctggcattgc tacaggcaag |
| 32341 | tagtgaacta actcaagcag gtaaacaaga atttgacaaa ttactcacaa aactgaatat |
| 32401 | cccccagcac ttacacgaag aagctaagtt cggatcaact ttattaatga caatcttttt |
| 32461 | acttggtttt tggttggctc taccgtcaat ttcccaactc tacaaccgtc aaggactcag |
| 32521 | taattaccta cagggaaaat taggagcagc agaacaagac taccttaaag caatttcctt |
| 32581 | ggatgccgat aatgcagatg ctcattataa tcttggcaat ctttacgagg atttacaaga |
| 32641 | ttttgagaaa gcacggaagc attatcaaat cgcagtggga agtgacgttc ctgatgctta |
| 32701 | caataatcta gctcgcttat acatccagga taaaaaatat tcccaggcta gagctttgct |
| 32761 | acaaaatgga ctagtaaaag ctaaagacct tcccgaagtc agatacagcc tgtttaaaaa |
| 32821 | tttaggatgg gtaaggtttg agcaaggcag atatgaagaa gcacaactaa acttacaagc |
| 32881 | agcaattggt attgcaaata acccagaggt agcaaagaat atccaaaata ctggtgcggc |
| 32941 | gtattgtatc ttagcgcagg taatggagcg gcaaaaacaa ccggccgctt tggagcaatg |
| 33001 | gcagaagtgc tgtgaacaag gttctactct caatgctgat gaagacacat ggttgcatct |
| 33061 | agctcggcaa aaattacaaa aggcaggaag agtatgcaaa tagaccggaa aaagattagt |
| 33121 | ttcttagcca ttgtactaag cctacctttt atgacggtag caacacctgg gctgagtcaa |
| 33181 | cagaatagtc taaattttat ctccgaagtt aagggagatg tcaaaattca acgttctgga |
| 33241 | cgcaagaacc atcaaaaagc atatggtggt gaactactag attcttctga ttctttgcga |
| 33301 | ttgggaaaag gtgcttctac aaaagttgta tgtaataact tatccatttg gaatattaaa |
| 33361 | tctccaggga aattttcagt ctctagtggt tgtccatcca ctggaaaacc agttcttata |
| 33421 | agaaatggta gtgaacgcgc tccgactcgc gctcccaatg acccgacaat tccctacatc |
| 33481 | attagtccgc gtgatacagc aattttgagt ggcaaaccta ctctgcgttg gaatgctgtt |
| 33541 | aagggtgcaa ccagctataa ggtgacgtta cgcggccctg gtgttaattg gacaactaat |
| 33601 | gtgaagcagt cagaagttgt ttattctggt caagaacctc tacagccagg ttttcgttat |
| 33661 | cgggtagttg tgactgctga taatggagca acttctgaga gtgatgcacc tgttggattc |
| 33721 | aatttactga gtgaggggga ggctcaacag gtaaaagctg aaattacaca attacaaaag |
| 33781 | caaccgctaa ctgatgaatc taaaaatctt gctttggcat atttgtacca aagcaagaat |
| 33841 | ttaaatgctg tggcaattga tttattagca ggcttagtta agcaagaaaa ccaaagcaca |
| 33901 | gcagtttatc agcttttagg tagcctctac caacaggtag gtttacatcg attagctaaa |
| 33961 | gaacattact taactgcgct caagttagct aagacaaaca aaagtctgga agcgcaagca |
| 34021 | atcatccaaa ccagcttagg agaaattgat atagttcttg acaacttgca gcaggcgttt |
| 34081 | gagtcattgc aagctgctca aaataactat cgtgccttgg gagatgagca acaagtacag |
| 34141 | caactacagc agaaactaga tgacttgaaa ggaaggctac catcatgaat aacctgcgaa |
| 34201 | ctagacaatt agtaacccta acaattttgg tagcaacagc tggattatgg gtgttaatgc |
| 34261 | caaagcccac aattgggcaa tcaacccctc ctgtttctgc tgctcagtct gcggaattgg |
| 34321 | cggaagtaga aagactcaat caacaggtga ttcaacttta tgaaaaaggc aaatatagtg |
| 34381 | aagcaattcc cttagcagag cgtgcattgg ctatccgtaa gcaaaaacta ggcgatgagc |
| 34441 | atccggatgt ggcaagnnnn nnnnnnnnnn nnnnnnnnnn nnnnnnnnnn nnnnnnnnnn |
| 34501 | nnnnnnnnnn nnnnnnnnnn nnnnnnnnnn nnnnnnnnnn nnnnnnnnnn nnnnnnnnnn |
| 34561 | nnnnnnnnnn nnnnnnnnnn nnnnnnnnnn nnnnnnnnnn nnnnnnnnnn nnnnnnnnnn |
| 34621 | nnnnnnnnnn nnnnnnnnnn nnnnnnnnnn nnnnnnnnnn nnnnnnnnnn nnnnnnnnnn |
| 34681 | nnnnnnnnnn nnnnnnnnnn nnnnnnnnnn nnnnnnnnnn nnnnnnnnnn nnnnnnnnnn |
| 34741 | nnnnnnnnnn nnnnnnnnnn nnnnnnnnnn nnnnnnnnnn nnnnnnnnnn nnnnnnnnnn |
| 34801 | nnnnnnnnnn nnnnnnnnnn nnnnnnnnnn nnnnnnnnnn nnnnnnnnnn nnnnnnnnnn |
| 34861 | nnnnnnnnnn nnnnnnnnnn nnnnnnnnnn nnnnnnnnnn nnnnnnnnnn nnnnnnnnnn |
| 34921 | nnnnnnnnnn nnnnnnnnnn nnnnnnnnnn nnnnnnnnnn nnnnnnnnnn nnnnnnnnnn |
| 34981 | nnnnnnnnnn nnnnnnnnnn nnnnnnnnnn nnnnnnnnnn nnnnnnnnnn nnnnnnnnnn |
| 35041 | nnnnnnnnnn nnnnnnnnnn nnnnnnnnnn nnnnnnnnnn nnnnnnnnnn nnnnnnnnnn |
| 35101 | nnnnnnnnnn nnnnnnnnnn nnnnnnnnnn nnnnnnnnnn nnnnnnnnnn nnnnnnnnnn |
| 35161 | nnnnnnnnnn nnnnnnnnnn nnnnnnnnnn nnnnnnnnnn nnnnnnnnnn nnnnnnnnnn |
| 35221 | nnnnnnnnnn nnnnnnnnnn nnnnnnnnnn nnnnnnnnnn nnnnnnnnnn nnnnnnnnnn |
| 35281 | nnnnnnnnnn nnnnnnnnnn nnnnnnnnnn nnnnnnnnnn nnnnnnnnnn nnnnnnnnnn |
| 35341 | nnnnnnnnnn nnnnnnnnnn nnnnnnnnnn nnnnnnnnnn nnnnnnnnnn nnnnnnnnnn |
| 35401 | nnnnnnnnnn nnnnnnnnnn nnnnnnnnnn nnnnnnnnnn nnnnnnnnnn nnnnnnnnnn |
| 35461 | nnnnnnnnnn nnnnnnnnnn nnnnnnnnnn nnnnnnnnnn nnnnnnnnnn nnnnnnnnnn |
| 35521 | nnnnnnnnnn nnnnnnnnnn nnnnnnnnnn nnnnnnnnnn nnnnnnnnnn nnnnnnnnnn |
| 35581 | nnnnnnnnnn nnnnnnnnnn nnnnnnnnnn nnnnnnnnnn nnnnnnnnnn nnnnnnnnnn |
| 35641 | nnnnnnnnnn nnnnnnnnnn nnnnnnnnnn nnnnnnnnnn nnnnnnnnnn nnnnnnnnnn |
| 35701 | nnnnnnnnnn nnnnnnnnnn nnnnnnnnnn nnnnnnnnnn nnnnnnnnnn nnnnnnnnnn |
| 35761 | nnnnnnnnnn nnnnnnnnnn nnnnnnnnnn nnnnnnnnnn nnnnnnnnnn nnnnnnnnnn |
| 35821 | nnnnnnnnnn nnnnnnnnnn nnnnnnnnnn nnnnnnnnnn nnnnnnnnnn nnnnnnnnnn |
| 35881 | nnnnnnnnnn nnnnnnnnnn nnnnnnnnnn nnnnnnnnnn nnnnnnnnnn nnnnnnnnnn |
| 35941 | nnnnnnnnnn nnnnnnnnnn nnnnnnnnnn nnnnnnnnnn nnnnnnnnnn nnnnnnnnnn |
| 36001 | nnnnnnnnnn nnnnnnnnnn nnnnnnnnnn nnnnnnnnnn nnnnnnnnnn nnnnnnnnnn |
| 36061 | nnnnnnnnnn nnnnnnnnnn nnnnnnnnnn nnnnnnnnnn nnnnnnnnnn nnnnnnnnnn |
| 36121 | nnnnnnnnnn nnnnnnnnnn nnnnnnnnnn nnnnnnnnnn nnnnnnnnnn nnnnnnnnnn |
| 36181 | nnnnnnnnnn nnnnnnnnnn nnnnnnnnnn nnnnnnnnnn nnnnnnnnnn nnnnnnnnnn |
| 36241 | nnnnnnnnnn nnnnnnnnnn nnnnnnnnnn nnnnnnnnnn nnnnnnnnnn nnnnnnnnnn |
| 36301 | nnnnnnnnnn nnnnnnnnnn nnnnnnnnnn nnnnnnnnnn nnnnnnnnnn nnnnnnnnnn |
| 36361 | nnnnnnnnnn nnnnnnnnnn nnnnnnnnnn nnnnnnnnnn nnnnnnnnnn nnnnnnnnnn |
| 36421 | nnnnnnnnnn nnnnnnnnnn nnnnnnnnnn nnnnnnnnnn nnnnnnnnnn nnnnnnnnnn |
| 36481 | nnnnnnnnnn nnnnnnnnnn nnnnnnnnnn nnnnnnnnnn nnnnnnnnnn nnnnnnnnnn |
| 36541 | nnnnnnnnnn nnnnnnnnnn nnnnnnnnnn nnnnnnnnnn nnnnnnnnnn nnnnnnnnnn |
| 36601 | nnnnnnnnnn nnnnnnnnnn nnnnnnnnnn nnnnnnnnnn nnnnnnnnnn nnnnnnnnnn |
| 36661 | nnnnnnnnnn nnnnnnnnnn nnnnnnnnnn nnnnnnnnnn nnnnnnnnnn nnnnnnnnnn |
| 36721 | nnnnnnnnnn nnnnnnnnnn nnnnnnnnnn nnnnnnnnnn nnnnnnnnnn nnnnnnnnnn |
| 36781 | nnnnnnnnnn nnnnnnnnnn nnnnnnnnnn nnnnnnnnnn nnnnnnnnnn nnnnnnnnnn |
| 36841 | nnnnnnnnnn nnnnnnnnnn nnnnnnnnnn nnnnnnnnnn nnnnnnnnnn nnnnnnnnnn |
| 36901 | nnnnnnnnnn nnnnnnnnnn nnnnnnnnnn nnnnnnnnnn nnnnnnnnnn nnnnnnnnnn |
| 36961 | ntagcttgaa taatctggca ttgctgtact ggaaccaaga tgacacaagc cgcgctcttg |
| 37021 | agtttttaac tcaaggaata acagttcaag aacacaacct cactctcaac ttggctgtcg |
| 37081 | gttttgagcg acaaaaacgc gattacgtca agactatctc tgctaccaca gatgcttcca |
| 37141 | tttcactgca cctcaatggt gctgctaata accccagtgc tgctaattta gctctaagca |
| 37201 | cagtcttaca acgcaaagga cgcatcttgg atgtcctcac taatagccag caaattctac |
| 37261 | gtcaacgcct agacccagaa agcaagactc tgttaacgaa gttagcaaac acccaaactc |
| 37321 | aactagccaa tctattttat aaccgaccag agaaacttcc tcttgagcag tatcggactc |
| 37381 | aaattgccca actggaaacc caagcaaaac aactagaaga tcaactgagt cgtcgcagcc |
| 37441 | aagaatttcg tactcagtcc cagcttgtca ccattgaaac tgttcaaaaa ttaattcctg |
| 37501 | ctgatggtgc tttggtagaa atagtccgct atcgacctgt taatcttaaa gcagcacctg |
| 37561 | aagatcagtt tggtaaacct cgttacgcag tatacgtcct tccagcaaaa ggacaaccta |
| 37621 | aaggtattga tttaggcgat gctgaaacaa ttgagcaaac tctcatccaa ttacgcaagg |
| 37681 | acttgcaaga tcaaaaaact cctatttctc aaatgaaaca gtcggctcgt gagttggatt |
| 37741 | caattctcat gcagcctgta cgcaaacttt tgggcaatac tcgcaaaatt ttactctctc |
| 37801 | cagacagtca gcttcacctc attccctttg aggcactggt ggatgaaaat aaccgctacc |
| 37861 | tactagaaaa ctattctttc acctacctta cctctggacg agacttgctc agactgcaaa |
| 37921 | acaaatctgc cagtaaacag ccgccagtta ttattgctga ccccaatttc aatcgtcctg |
| 37981 | gcaaattagt tgcgcgtcag ccttatgcta atagtattga tgcaaatacc cgctctatag |
| 38041 | acctgtccca gaaagtattt ccccccctcc ccggtacagc acaagaagcc aaggccattg |
| 38101 | ccagtctatt aaaaatccaa cctcttacag acactaaggc tactgagggt gctgtcaaac |
| 38161 | aagttaacag ccccaggatt ctgcatattg ccacacacgg tatttttgaa aatcccccag |
| 38221 | caaacgaagg gaaaaccaca cttgaagata atccgctact gcaatcagga ttagttctag |
| 38281 | ctggcttaaa acctcgtcag agtgctggtg aagatgggat tctcactgcc ctggaaacaa |
| 38341 | ctacactcaa tctagttggt acaaaactgg tggtactctc tgcctgtaat acaggtttgg |
| 38401 | gtaatattaa tgcaggtgaa ggcgtttatg gtttgcgtcg cgctttagtc attgctggtt |
| 38461 | ctgaaagcca agtcatcagc ctgtggaaag tagcagatga tgctactaaa gattttatgg |
| 38521 | tggcttacta ccaaagatta ttagataatc aaggacgcag tgaagcacta cgccaaacgc |
| 38581 | aactagagat gttaaagggt gataagtatc agcatcccta cttctgggct gcgtttatcc |
| 38641 | cttctggtga ttggaaaccg atgggtaagt gagttagttt tgagagcttt ggcgatcctt |
| 38701 | ccatattatc ggctcaacat gaaatagagt gcgatcgcct ccctcgaaac taaactgttg |
| 38761 | attcagcttt gtactgggaa tgcgatgcga tcgccactaa atcttggagt aaatattccc |
| 38821 | aaatccttac ggggaagtaa tttgcaaaat tgtacaactt ttttatttct ttttggatag |
| 38881 | tagcctgatg aaattgtaga actttatttt ttcaagactt cttagtaatc aacgatataa |
| 38941 | caatccttgt agccgcgcaa attatacaac tttattatat ttatacagag gttgtagaga |
| 39001 | aataattcga ttcaacaata aagatgttaa aaataacctc taaagaatta ctaaaataat |
| 39061 | tgcaaataga ggttattttt attggaacag agacaaaaca ggtttttcaa aggctagagt |
| 39121 | ctttcgatat caggtaaaat ttgataaatc tgctgccagt gaggacttat ccagccaaca |
| 39181 | atacgcacta cttctcgtaa aattacgata gacaaaccgc attcaacctt ctcaaaagca |
| 39241 | gttgcacaat ctgttaatag acttacgaaa ctatctagtt tttgtaattt catctttgct |
| 39301 | aatccatctg caaattgccc aatttgagct aatactacac taatttcgcc taaactctct |
| 39361 | tcgtccatat ccttacatat taaatcccaa tctttcttga tttctgaaaa tatctcctca |
| 39421 | gcttctactt cttgcccttg ttgataaaag gcttctacta aactatataa agctactatc |
| 39481 | ttttctggtg tatcttcaat caagcctgct actttcttag ctttagaaca aaagccaaat |
| 39541 | cttgctaaga cttgtgttaa attctctaat aaaaatggtt gatcaaaagg attatccatt |
| 39601 | tcaagaatca tttcttctat ttcagttagc aatatatctg ctctttctgc gtattcaccc |
| 39661 | ttataaaggc tatttaataa tattgttaaa gcttggattt ggaaaaaacc agtttcaagt |
| 39721 | tttctggcta ttctttcggt agctaaaaat agagtattag catttttggg tttgtttata |
| 39781 | caattaaatg cgactgccaa atcagtcatt aatcataagc gataaggctc att |

Features of the components in the D328 locus above, e.g., TnsA, TnsB, TnsC, TniQ (TnsD), Cas5, Cas6, Cas7, Cas8, and CRISPR clusters are shown below.

| FEATURES | Location/Qualifiers |
|---|---|
| glmS | 159 . . . 2033 |
| | /created_by = “makoto” |
| | /label = “glmS” |
| CDS | 159 . . . 2033 |
| | /product = “NA > c1_19425” |
| | /gene = “NA > c1_19425” |
| | /locus_tag = “NA > c1_19425” |
| | /label = “NA > c1 19425 CDS” |
| ITR | 2058 . . . 2124 |
| | /created_by = “makoto” |
| | /label = “RE” |
| motif_3 | 2066 . . . 2084 |
| | /Mismatches = 0 |
| | /%_Identity = 100 |
| | /Motif = “[ATC][AC][AT]AA[AT]AAAGTT[GC]TA[CT]AAT” |
| | /annotation_group = “AttTn7_cluster_3: 11,075,376 → |
| | 11,075,394” |
| | /label = “AttTn7_cluster_3” |
| motif_3 | 2086 . . . 2104 |
| | /Mismatches = 0 |
| | /%_Identity = 100 |
| | /Motif = “[ATC][AC][AT]AA[AT]AAAGTT[GC]TA[CT]AAT” |
| | /annotation_group = “AttTn7_cluster_3: 11,075,396 → |
| | 11,075,414” |
| | /label = “AttTn7_cluster_3” |
| motif_3 | 2106 . . . 2124 |
| | /Mismatches = l |
| | /%_Identity = “94.73684210526316” |
| | /Motif = “[ATC][AC][AT]AA[AT]AAAGTT[GC]TA[CT]AAT” |
| | /annotation_group = “AttTn7_cluster_3: 11,075,416 → |
| | 11,075,434” |
| | /label = “AttTn7_cluster_3” |
| tnsA | 2170 . . . 2985 |
| | /product = “1-272\|id: 62.500@bs: 371” |
| | /gene = “1-272\|id: 62.500@bs: 371” |
| | /locus_tag = “1-272\|id: 62.500@bs: 371” |
| | /label = “1-272\|id: 62.500@bs: 371 tnsA” |
| CDS | 2170 . . . 2988 |
| | /product = “tnsA_13 > c1_27484” |
| | /gene = “tnsA_13 > c1_27484” |
| | /locus_tag = “tnsA_13 > c1_27484” |
| | /label = “tnsA 13 > c1 27484 CDS” |
| tnsB | 3010 . . . 5232 |
| | /product = “1-742\|id: 67.564@bs: 1041” |
| | /gene = “1-742\|id: 67.564@bs: 1041” |
| | /locus_tag = “1-742\|id: 67.564@bs: 1041” |
| | /modified_by = “makoto” |
| | /label = “29-B 1-742\|id: 67.564@bs: 1041 tnsB” |
| CDS | 3010 . . . 5238 |
| | /product = “tnsB_14 > c1_26522” |
| | /gene = “tnsB_14 > c1_26522” |
| | /locus_tag = “tnsB_14 > c1_26522” |
| | /label = “tnsB 14 > c1 26522 CDS” |
| tnsC | 5207 . . . 6904 |
| | /product = “2-568\|id: 59.331@bs: 688” |
| | /gene = “2-568\|id: 59.331@bs: 688” |
| | /locus_tag = “2-568\|id: 59.331@bs: 688” |
| | /label = “2-568\|id: 59.331@bs: 688 tnsC” |
| CDS | 5222 . . . 6910 |
| | /product = “NA > c1_6196” |
| | /gene = “NA > c1_6196” |
| | /locus_tag = “NA > c1_6196” |
| | /label = “NA > c1 6196 CDS” |
| tniQ | 6915 . . . 7868 |
| | /product = “1-309\|id: 25.731@bs: 85.1” |
| | /gene = “1-309\|id: 25.73 l@bs: 85.1” |
| | /locus_tag = “1-309\|id: 25.731@bs: 85.1” |
| | /label = “1-309\|id: 25.731@bs: 85.1 tniQ” |
| CDS | 6915 . . . 7862 |
| | /product = “NA > c1_2402” |
| | /gene = “NA > c1_2402” |
| | /locus_tag = “NA > c1_2402” |
| | /label = “NA > c1 2402 CDS” |
| CDS | 8023 . . . 8691 |
| | /product = “NA > c1_11380” |
| | /gene = “NA > c1_11380” |
| | /locus_tag = “NA > c1_l 1380” |
| | /label = “NA > c1 11380 CDS” |
| cas6 | 8047 . . . 8688 |
| | /product = “15-227\|id: 63.084@bs: 281” |
| | /gene = “15-227\|id: 63.084@bs: 281” |
| | /locus_tag = “15-227\|id: 63.084@bs: 281” |
| | /label = “15-227\|id: 63.084@bs: 281 cas6” |
| cas8 | 8709 . . . 10238 |
| | /product = “l-512\|id: 64.453@bs: 696” |
| | /gene = “1-512\|id: 64.453@bs: 696” |
| | /locus_tag = “1-512\|id: 64.453@bs: 696” |
| | /label = “1-512\|id: 64.453@bs: 696 cas8” |
| CDS | 8709 . . . 10241 |
| | /product = “devT_19 > c1_6205” |
| | /gene = “devT_19 > c1_6205” |
| | /locus_tag = “devT_19 > c1_6205” |
| | /label = “devT 19 > c1 6205 CDS” |
| cas7 | 10299 . . . 11294 |
| | /product = “1-324\|id: 74.096@bs: 433” |
| | /gene = “1-324\|id: 74.096@bs: 433” |
| | /locus_tag = “1-324\|id: 74.096@bs: 433” |
| | /label = “1-324\|id: 74.096@bs: 433 cas7” |
| CDS | 10299 . . . 11297 |
| | /product = “devR_31 > c1_17591” |
| | /gene = “devR_31 > c1_17591” |
| | /locus_tag = “devR_31 > c1_17591” |
| | /label = “devR 31 > c1 17591 CDS” |
| cas5 | 11299 . . . 11928 |
| | /product = “1-210\|id: 87.619@bs: 390” |
| | /gene = “1-210\|id: 87.619@bs: 390” |
| | /locus_tag = “1-210\|id: 87.619@bs: 390” |
| | /label = “1-210\|id: 87.619@bs: 390 cas5” |
| CDS | 11299 . . . 11931 |
| | /product = “devS_23 > c1_25717” |
| | /gene = “devS_23 > c1_25717” |
| | /locus_tag = “devS_23 > c1_25717” |
| | /label = “devS 23 > c1 25717” |
| CRISPR | complement(12210 . . . 12538) |
| | /product = “5 CTGCTCAACGCCTAATGGCATCTAATGTTGAAGCAC” (SEQ ID NO: 75) |
| | /gene = “5 CTGCTCAACGCCTAATGGCATCTAATGTTGAAGCAC” (SEQ ID NO: 76) |
| | /locus_tag = “5 CTGCTCAACGCCTAATGGCATCTAATGTTGAAGCAC” (SEQ ID NO; 77) |
| | /label = “5 CTGCTCAACGCCTAATGGCATCTAATGTTGAAGCAC CRISPR” (SEQ ID NO: 78) |
| CDS | complement(12759 . . . 12989) |
| | /product = “NA > c1_2538” |
| | /gene = “NA > c1_2538” |
| | /locus_tag = “NA > c1_2538” |
| | /label = “NA > c1 2538 CDS” |
| CRISPR | complement(13076 . . . 13127) |
| | /product = “2 AGGCATTACACAATAC” (SEQ ID NO: 79) |
| | /gene = “2 AGGCATTACACAATAC” (SEQ ID NO: 80) |
| | /locus_tag = “2 AGGCATTACACAATAC” (SEQ ID NO: 81) |
| | /label = “2 AGGCATTACACAATAC CRISPR” (SEQ ID NO: 82) |
| tniQ | 13283 . . . 15190 |
| | /product = “1-636\|id: 91.352@bs: 1195” |
| | /gene = “1-636\|id: 91.352@bs: 1195” |
| | /locus_tag = “1-636\|id: 91.352@bs: 1195” |
| | /label = “1-636\|id: 91.352@bs: 1195 tniQ” |
| CDS | 13283 . . . 15193 |
| | /product = “NA > c1_1243” |
| | /gene = “NA > c1_1243” |
| | /locus_tag = “NA > c1_1243” |
| | /label = “NA > c1 1243 CDS” |
| CDS | complement(15236 . . . 15541) |
| | /product = “NA > c1_14703” |
| | /gene = “NA > c1_14703” |
| | /locus_tag = “NA > c1_14703” |
| | /label = “NA > c1 14703 CDS” |
| CDS | complement(15672 . . . 15965) |
| | /product = “NA > c1_2353” |
| | /gene = “NA > c1_2353” |
| | /locus_tag = “NA > c1_2353” |
| | /label = “NA > c1 2353 CDS” |
| CDS | 16481 . . . 17014 |
| | /product = “NA > noc1” |
| | /gene = “NA > noc1” |
| | /locus_tag = “NA > noc1” |
| | /label = “NA > noc1 CDS” |
| CDS | 17020 . . . 17835 |
| | /product = “NA > noc1” |
| | /gene = “NA > noc1” |
| | /locus_tag = “NA > noc1” |
| | /label = “NA > noc1 CDS” |
| CDS | 17819 . . . 18385 |
| | /product = “NA > noc1” |
| | /gene = “NA > noc1” |
| | /locus_tag = “NA > noc1” |
| | /label = “NA > noc1 CDS” |
| CDS | 18385 . . . 22101 |
| | /product = “NA > noc1” |
| | /gene = “NA > noc1” |
| | /locus_tag = “NA > noc1” |
| | /label = “NA > noc1 CDS” |
| CDS | 22104 . . . 26417 |
| | /product = “NA > noc1” |
| | /gene = “NA > noc1” |
| | /locus_tag = “NA > noc1” |
| | /label = “NA > noc1 CDS” |
| CDS | 26417 . . . 27772 |
| | /product = “NA > noc1” |
| | /gene = “NA > noc1” |
| | /locus_tag = “NA > noc1” |
| | /label = “NA > noc1 CDS” |
| CDS | 27863 . . . 28972 |
| | /product = “NA > noc1” |
| | /gene = “NA > noc1” |
| | /locus_tag = “NA > noc1” |
| | /label = “NA > noc1 CDS” |
| CDS | 28983 . . . 31016 |
| | /product = “NA > noc1” |
| | /gene = “NA > noc1” |
| | /locus_tag = “NA > noc1” |
| | /label = “NA > noc1 CDS” |
| CDS | 31850 . . . 33103 |
| | /product = “NA > noc1” |
| | /gene = “NA > noc1” |
| | /locus_tag = “NA > noc1” |
| | /label = “NA > noc1 CDS” |
| CDS | 33094 . . . 34188 |
| | /product = “NA > noc1” |
| | /gene = “NA > noc1” |
| | /locus_tag = “NA > noc1” |
| | /label = “NA > noc1 CDS” |
| CDS | 36966 . . . 38672 |
| | /product = “NA > noc1” |
| | /gene = “NA > noc1” |
| | /locus_tag = “NA > noc1” |
| | /label = “NA > noc1 CDS” |
| ITR | complement(38849 . . . 3 8987) |
| | /created_by = “makoto” |
| | /label = “Left End” |
| motif_3 | complement(38849 . . . 38867) |
| | /Mismatches = 0 |
| | /%_Identity = 100 |
| | /Motif = “[ATC][AC][AT]AA[AT]AAAGTT[GC]TA[CT]AAT” |
| | /annotation_group = “AttTn7_cluster_3: 11,112,159 ← |
| | 11,112,177” |
| | /label = “AttTn7_cluster_3” |
| motif_3 | complement(38893 . . . 38911) |
| | /Mismatches = 0 |
| | /%_Identity = 100 |
| | /Motif = “[ATC][AC][AT]AA[AT]AAAGTT[GC]TA[CT]AAT” |
| | /annotation_group = “AttTn7_cluster_3: 11,112,203 ← |
| | 11,112,221” |
| | /label = “AttTn7_cluster_3” |
| motif_3 | complements 8961 . . . 38979) |
| | /Mismatches = 0 |
| | /%_Identity = 100 |
| | /Motif = “[ATC][AC][AT]AA[AT]AAAGTT[GC]TA[CT]AAT” |
| | /annotation_group = “AttTn7_cluster_3: 11,112,271 ← |
| | 11,112,289” |
| | /label = “AttTn7_cluster_3” |
| CDS | complement(39114 . . . 39809) |
| | /product = “NA > noc1” |
| | /gene = “NA > noc1” |
| | /locus_tag = “NA > noc1” |
| | /label = “NA > noc1 CDS” |
| CDS | complement(39813 . . . >39833) |
| | /product = “NA > noc1” |
| | /gene = “NA > noc1” |
| | /locus_tag = “NA > noc1” |
| | /label = “NA > noc1 CDS” |

### Example 18

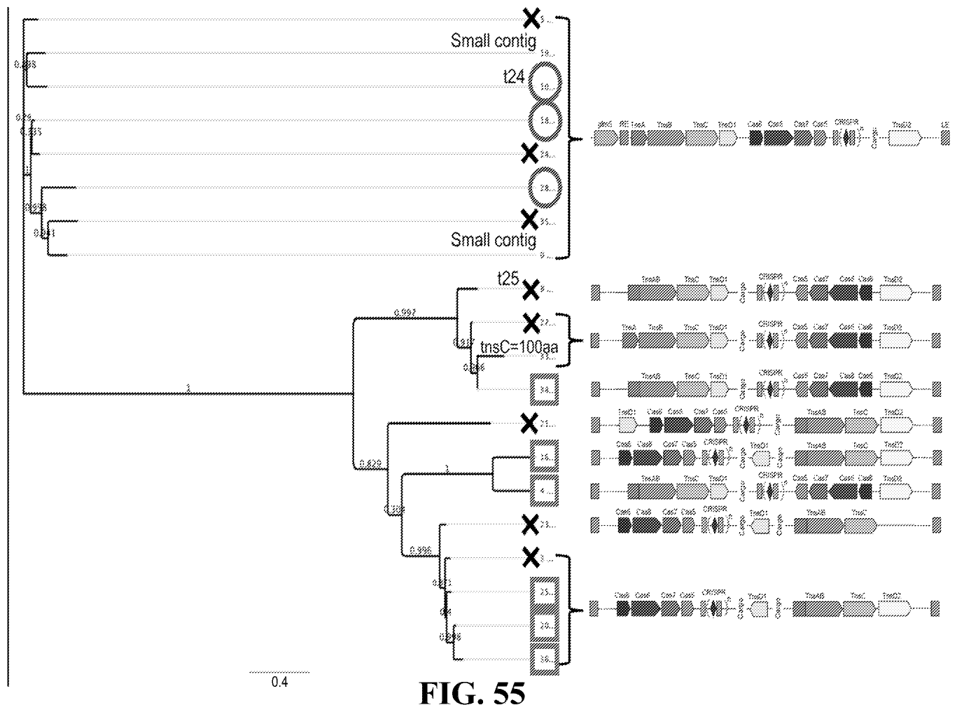

FIG. 55 shows exemplary CAST systems comprising Type IB Cas proteins.

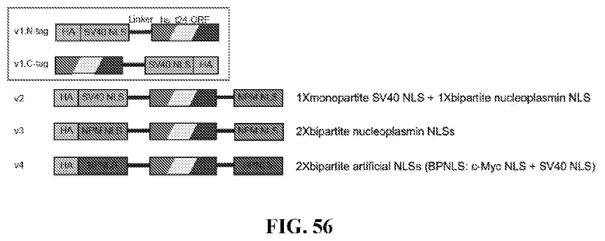

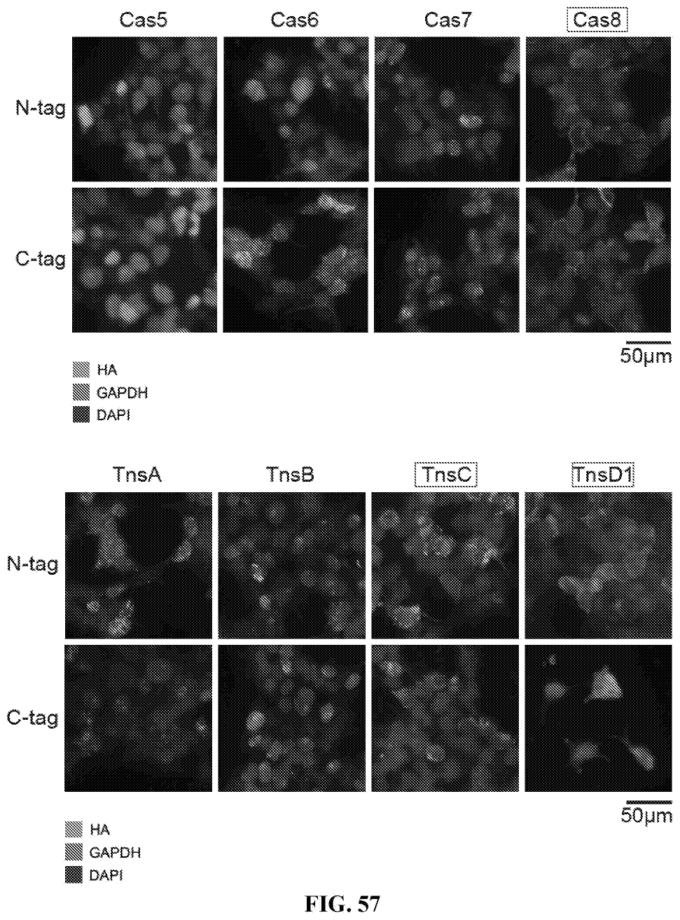

Applicants generated constructs expressing exemplary CAST (T24) attached with various NLS (FIG. 56). V1.N-tag and v1.C tag were mixed for all ORFs. V.4 NLS was a c-Myc and SV40 fusion artificial bipartite NLS.

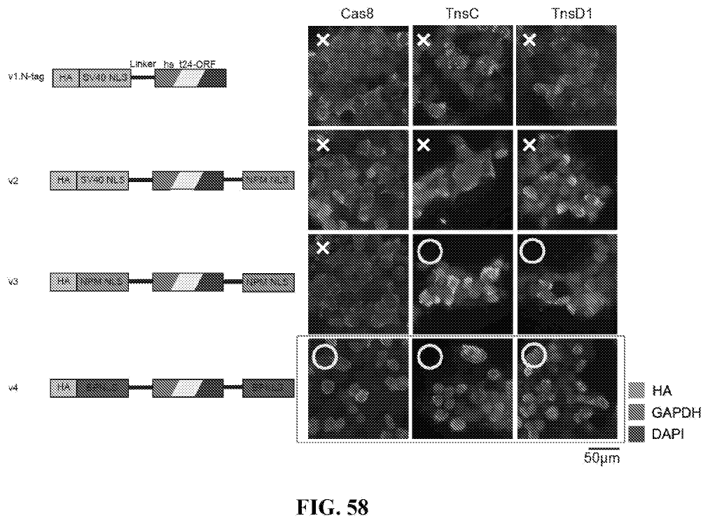

Subcellular localization of components of T24 attached with v1 NLS is shown in FIG. 57. Cas8, TnsC, and TnsD1 did not go to nucleus. Subcellular localization of components of T24 attached with other versions of NLS is shown in FIG. 58.

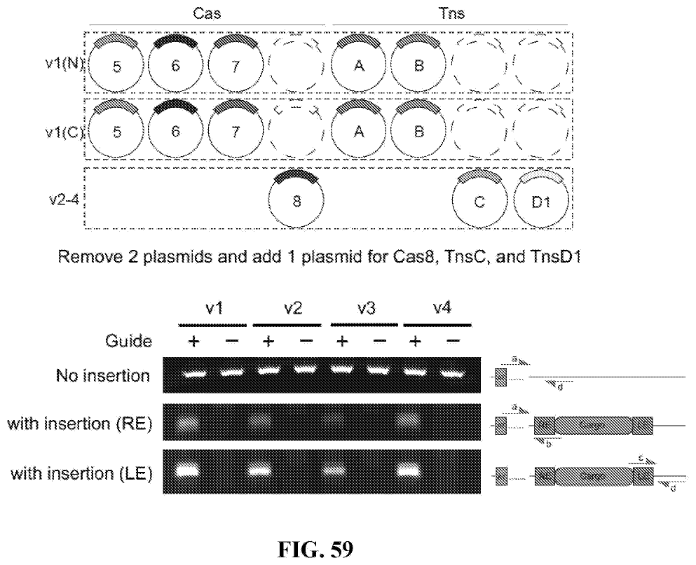

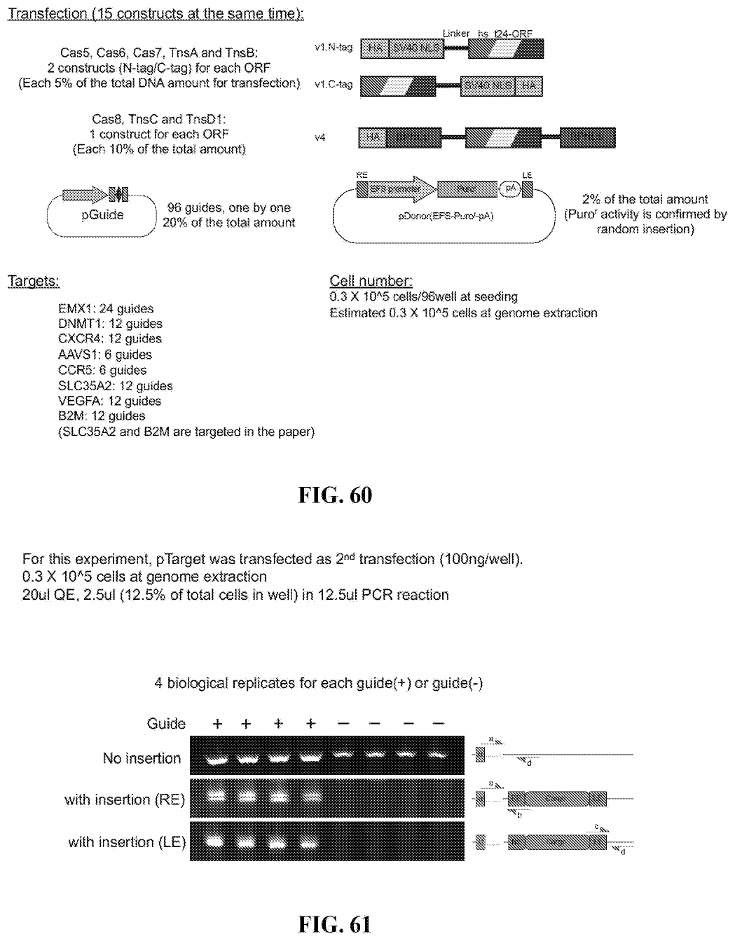

Applicants generated plasmids with the various NLS with T24, and tested their targeting activities (FIG. 59). V4 NLS for Cas8, TnsC, and TnsD1 was functional. FIG. 60 shows an exemplary experiment for testing mammalian genome targeting with t24. The targeting of the plasmids at 96-well format with QE (FIG. 61).

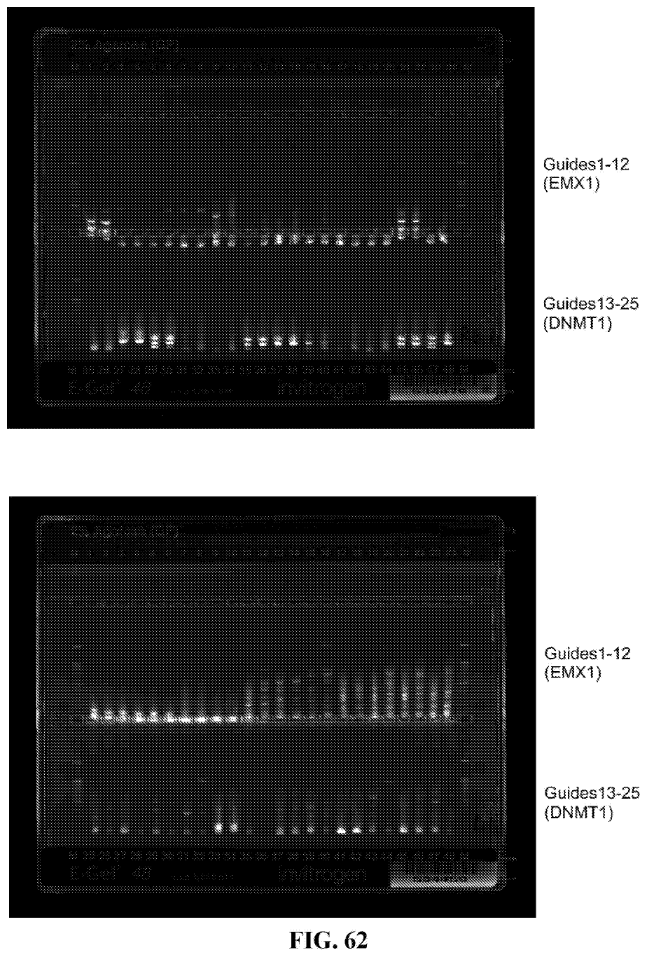

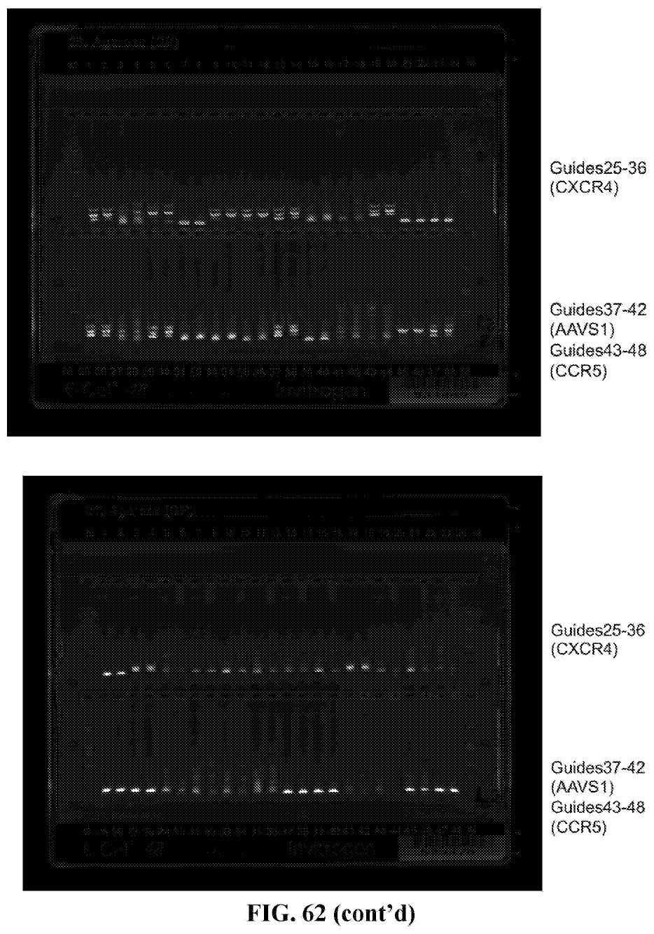

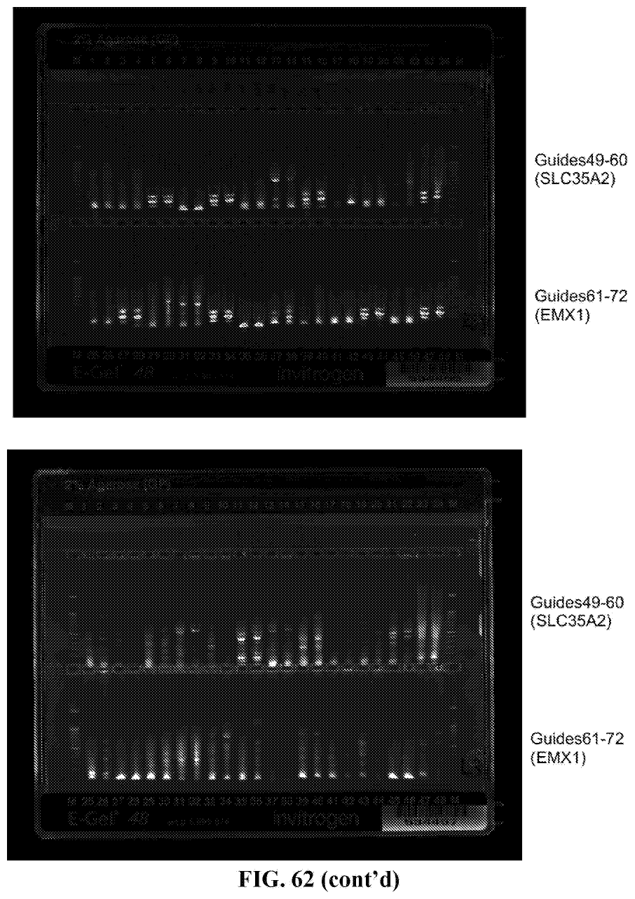

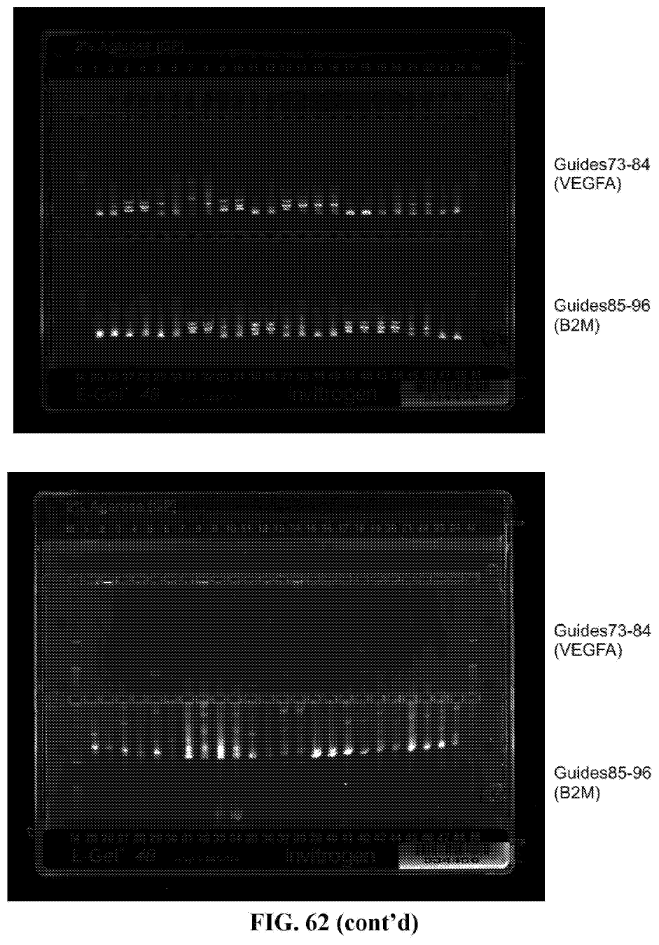

PCR experiments were performed to test the plasmids' effects on target genes (FIG. 62). 0.3×10⁵ cells were used at genome extraction. 40 μL QE, 10 μL (25% of total cells in well) in 50 μL PCR reaction. Odd number lanes ran all components transfection, and even number lanes ran controls (transfection without TnsD1). Expected amplicon size was 200˜400 bp for the guides.

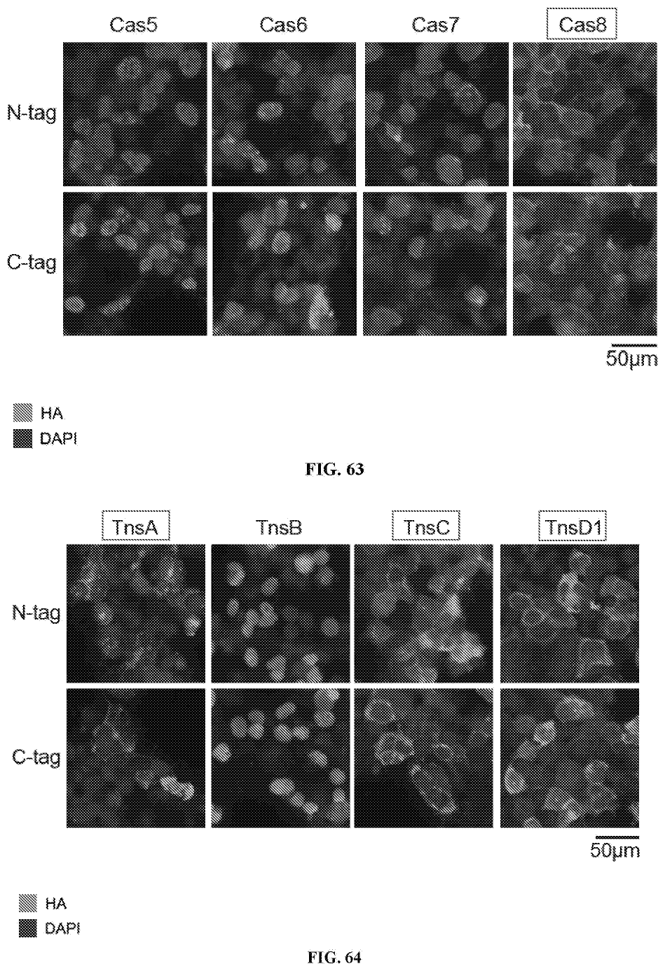

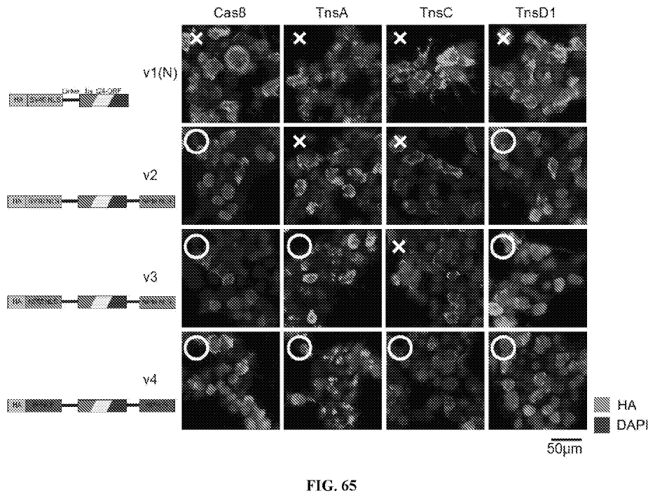

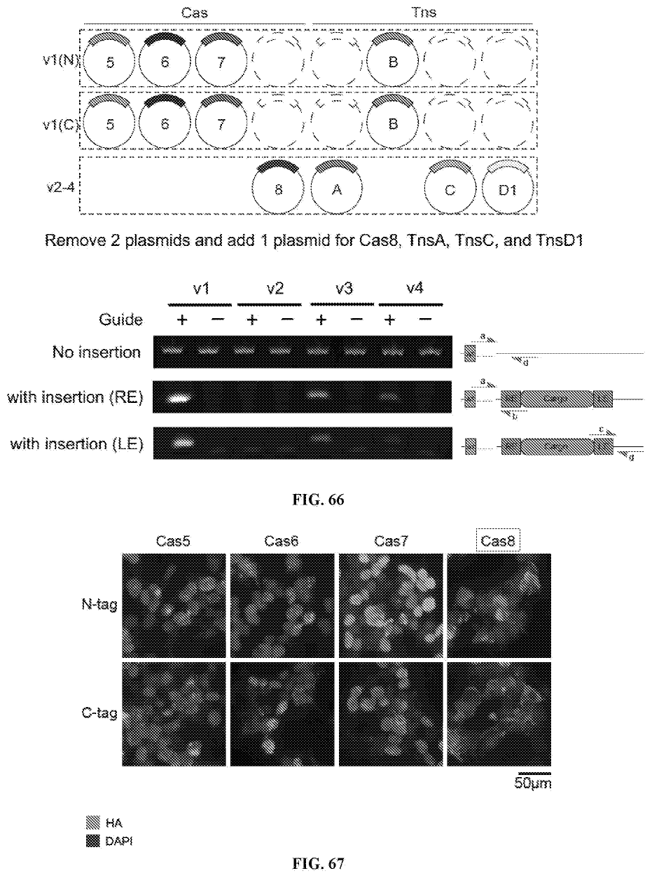

Subcellular localization of Cascade components of IB28 CAST system is shown in FIG. 63. Cas8 did not go to nucleus. Subcellular localization of Tns components of IB28 CAST system is shown in FIG. 64. TnsA, TnsC, and TnsD1 did not go to nucleus. Subcellular localization of components of IB28 CAST system with various versions of NLS is shown in FIG. 65. Applicants generated plasmids with the various NLS with IB28, and tested their targeting activities (FIG. 66).

Subcellular localization of Cascade components of v.1 of NLS for IB18 is shown in FIG. 67. Cas8 did not go to nucleus.

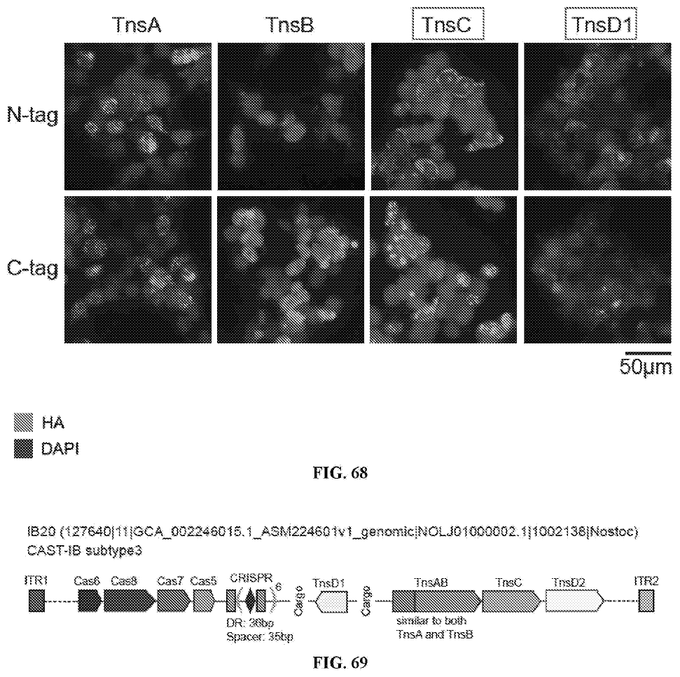

Subcellular localization of Tns components of v.1 of NLS for IB18 is shown in FIG. 68. N-tag TnsC and TnsD did not go to nucleus.

### Example 19

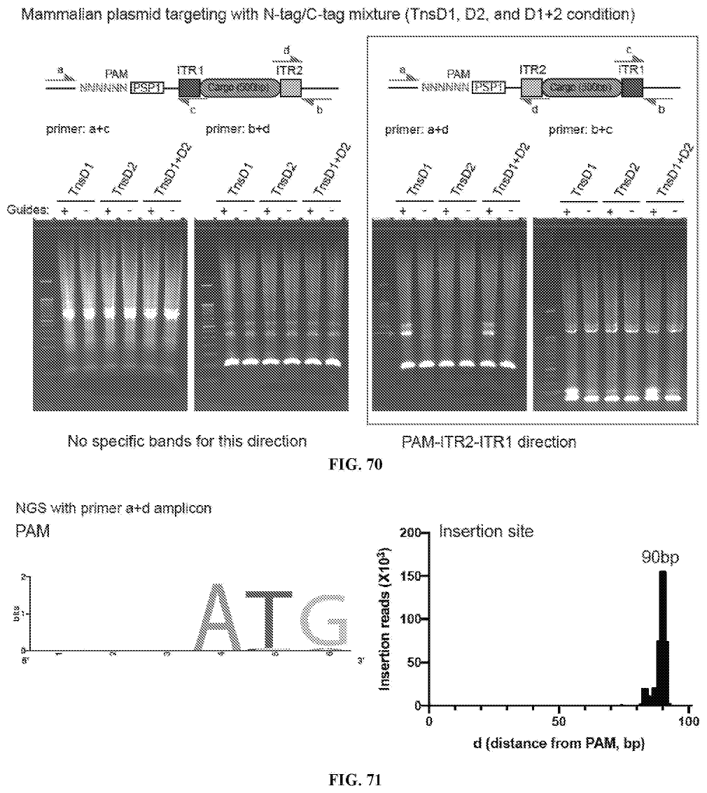

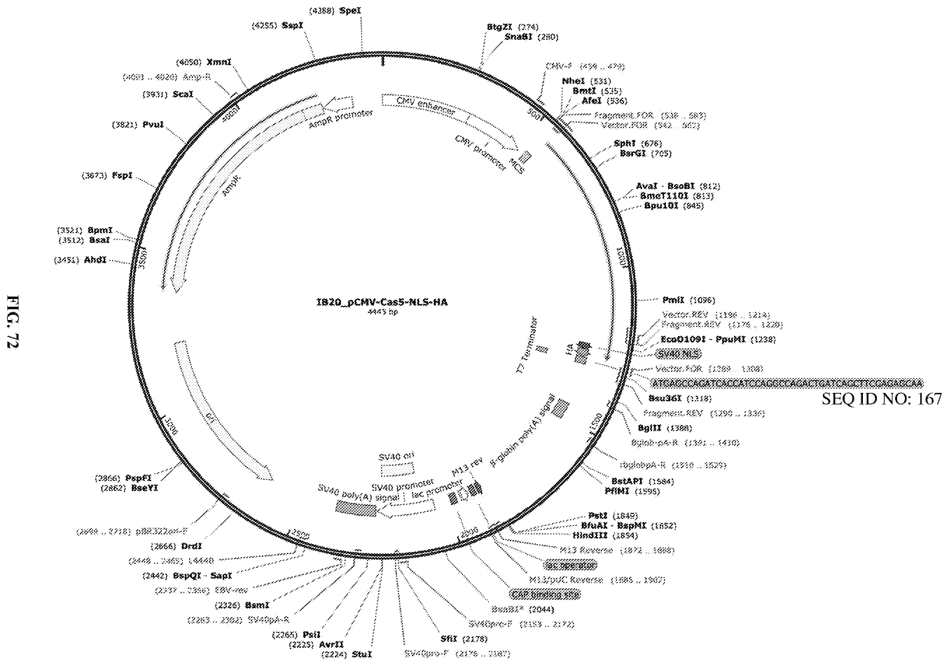

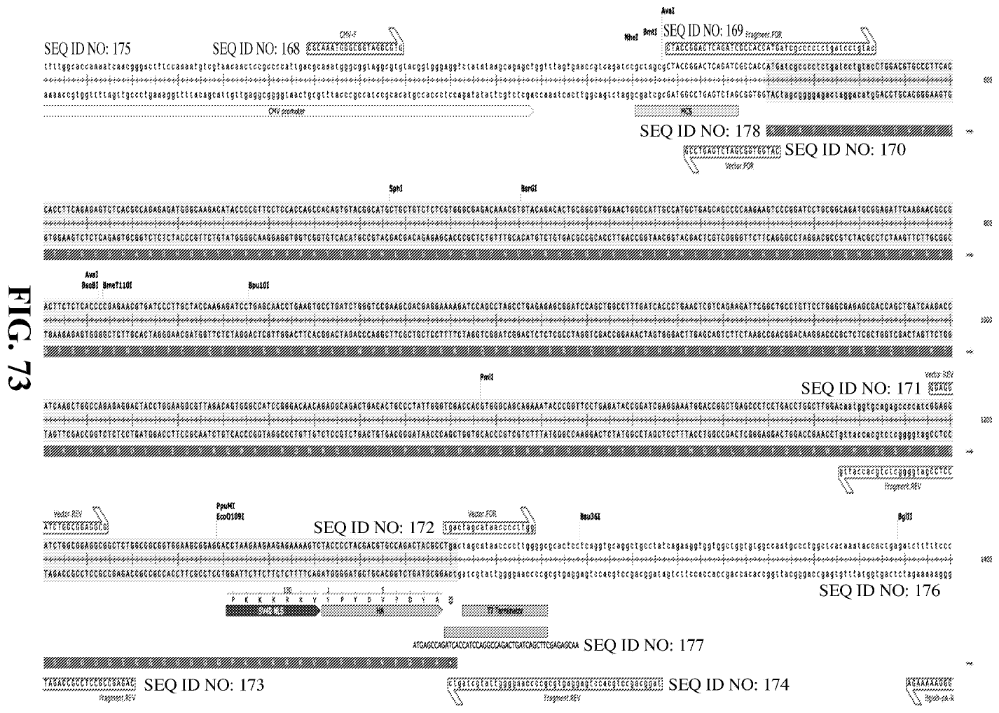

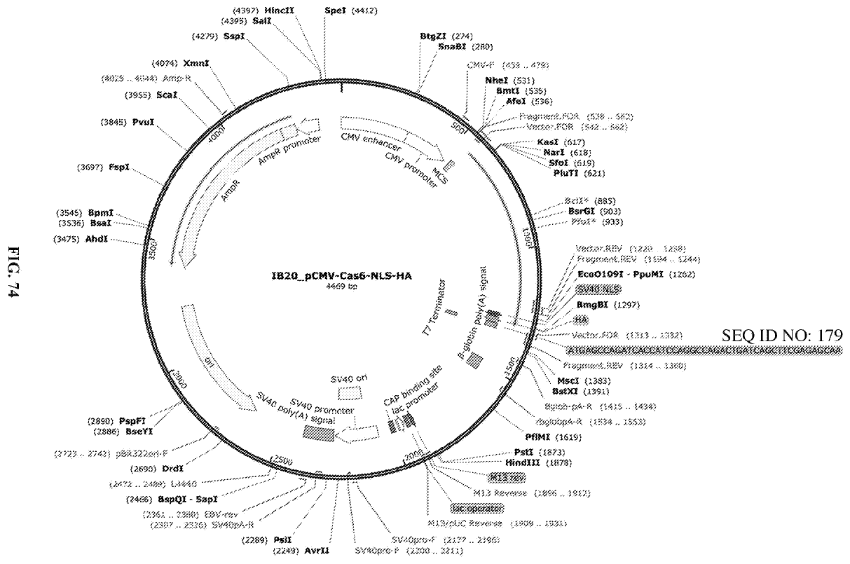

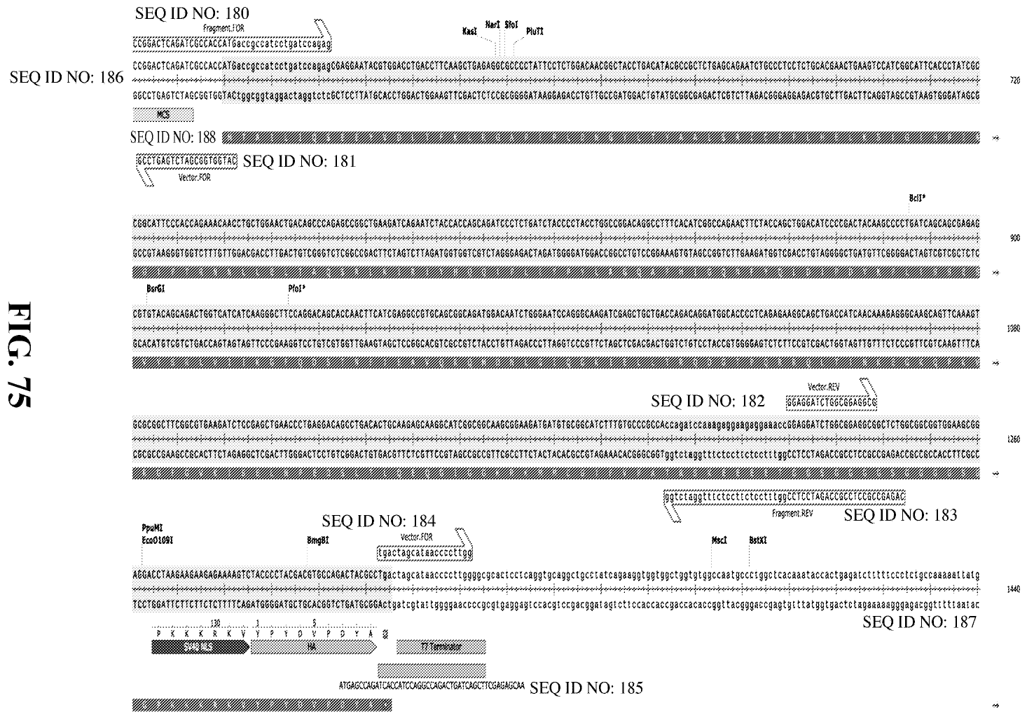

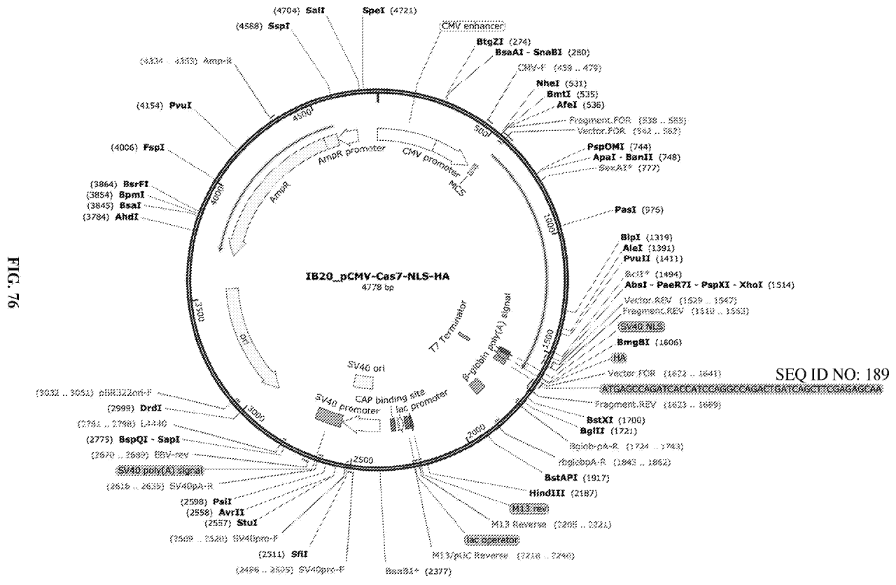

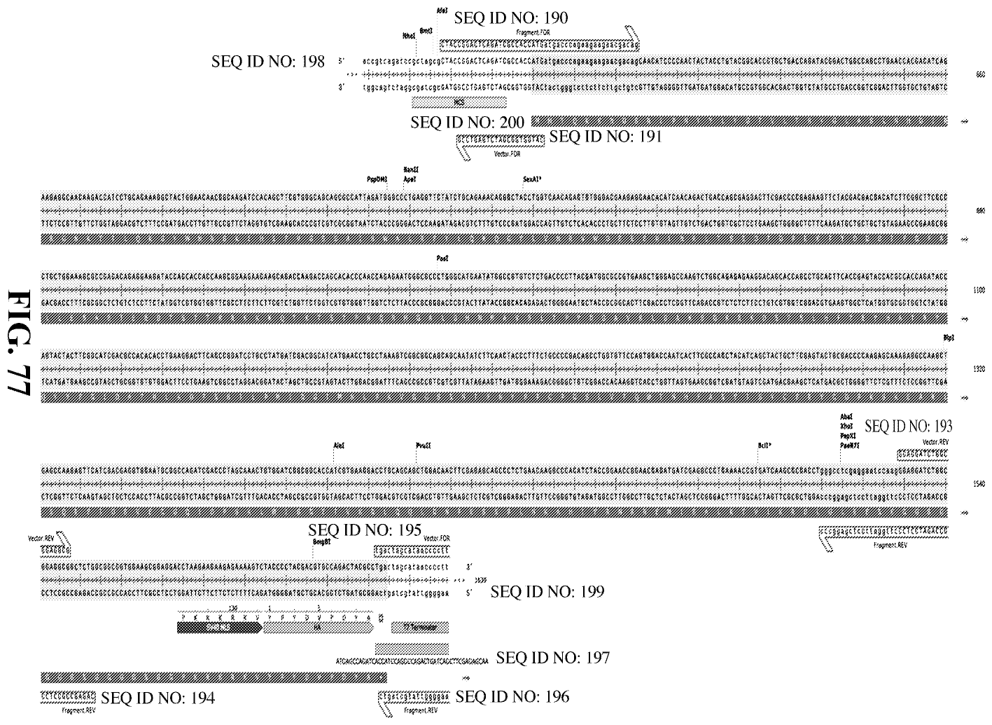

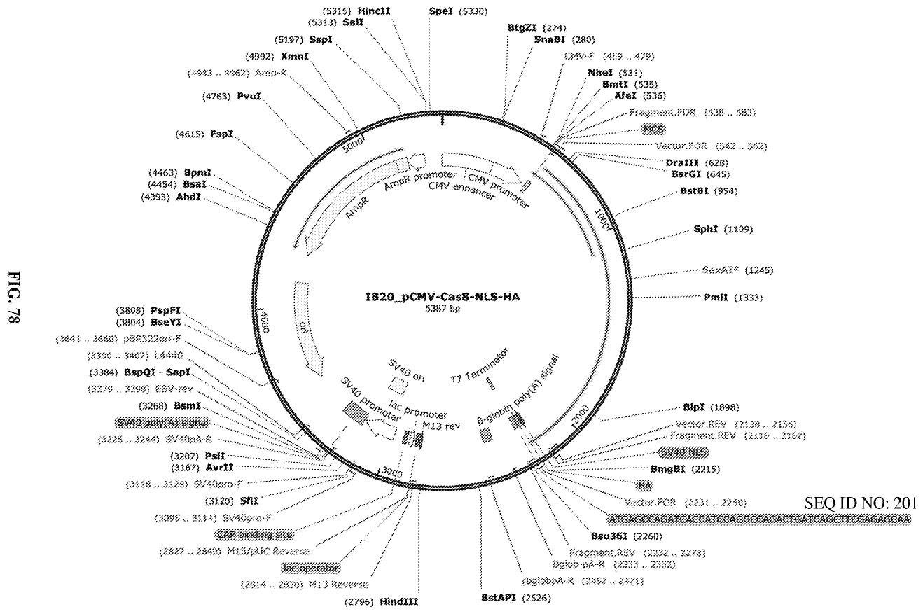

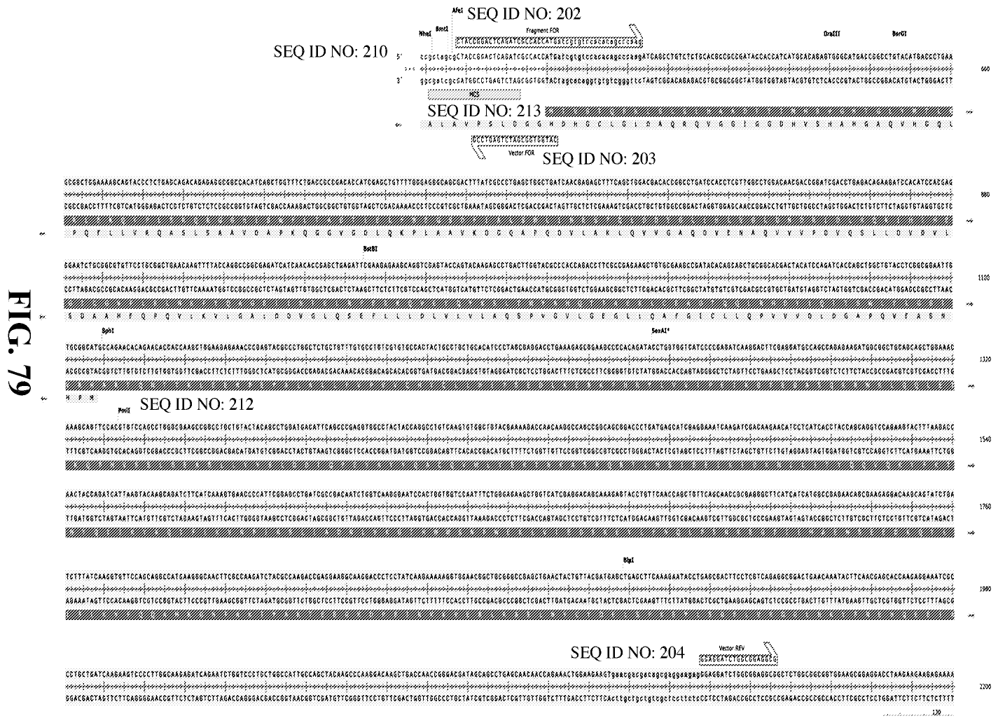

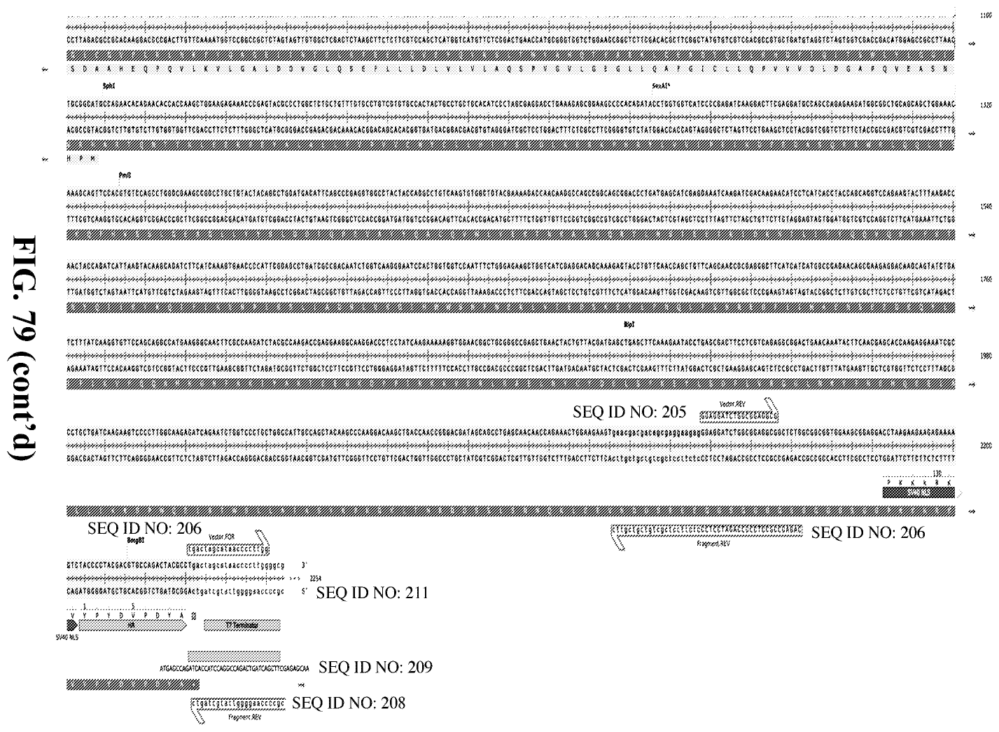

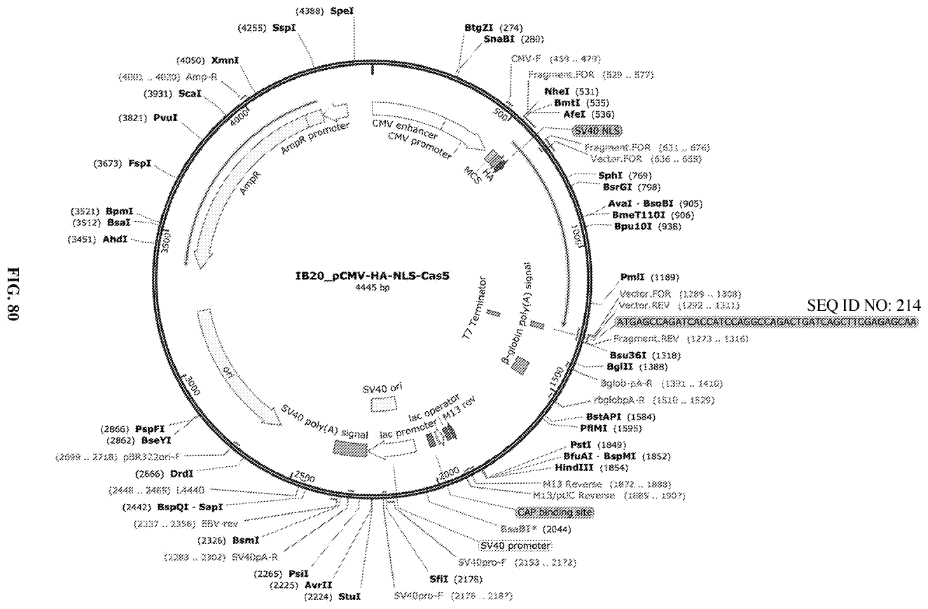

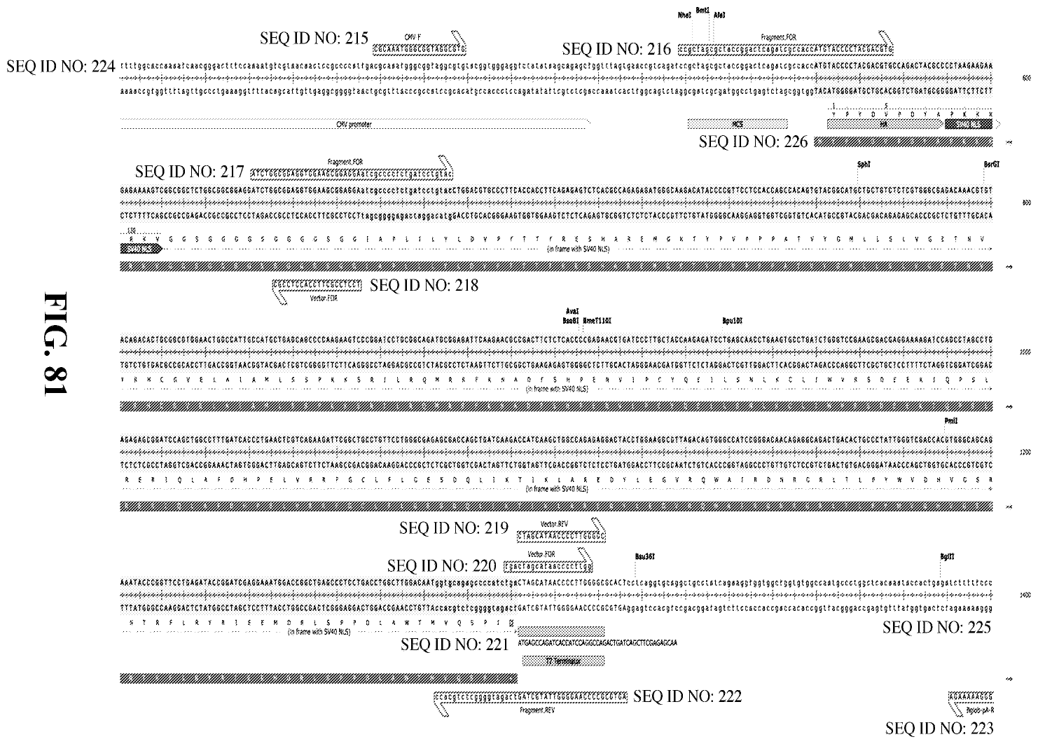

An exemplary CAST system IB20 was characterized. FIG. 69 shows the locus of IB20. FIG. 70 shows the function of D3 IB20 in mammalian cells. FIG. 71 shows the result from screening of PAM sequences.

The plasmids and relevant sequences are summarized below.

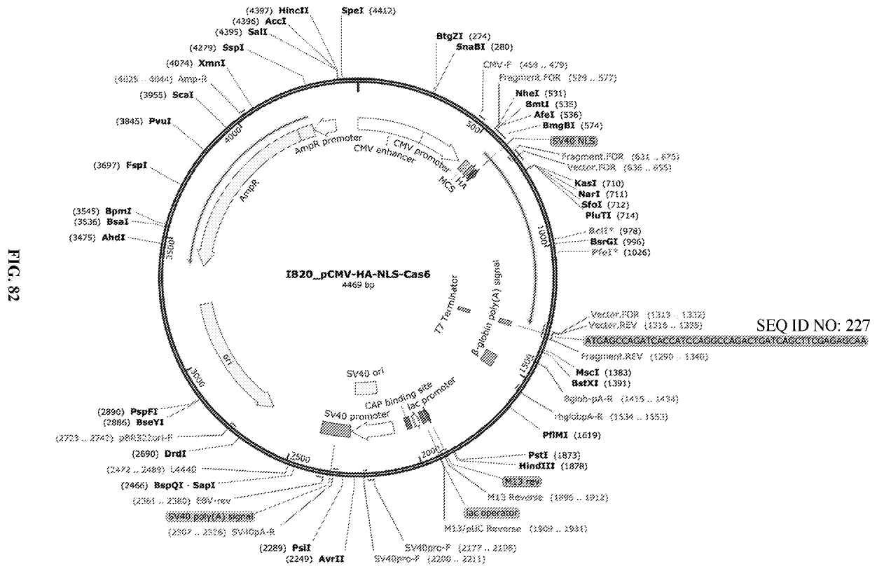

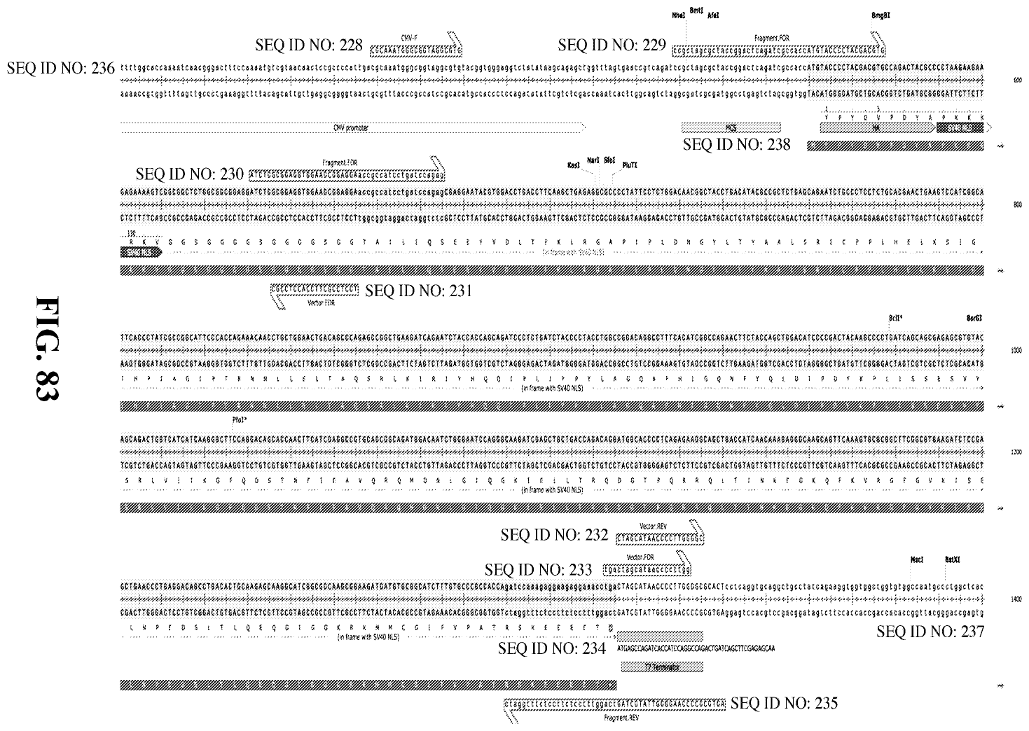

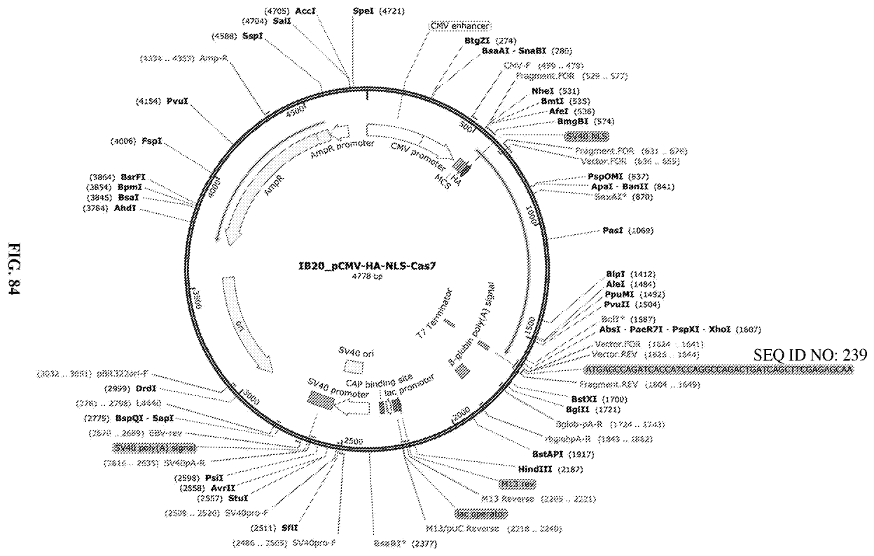

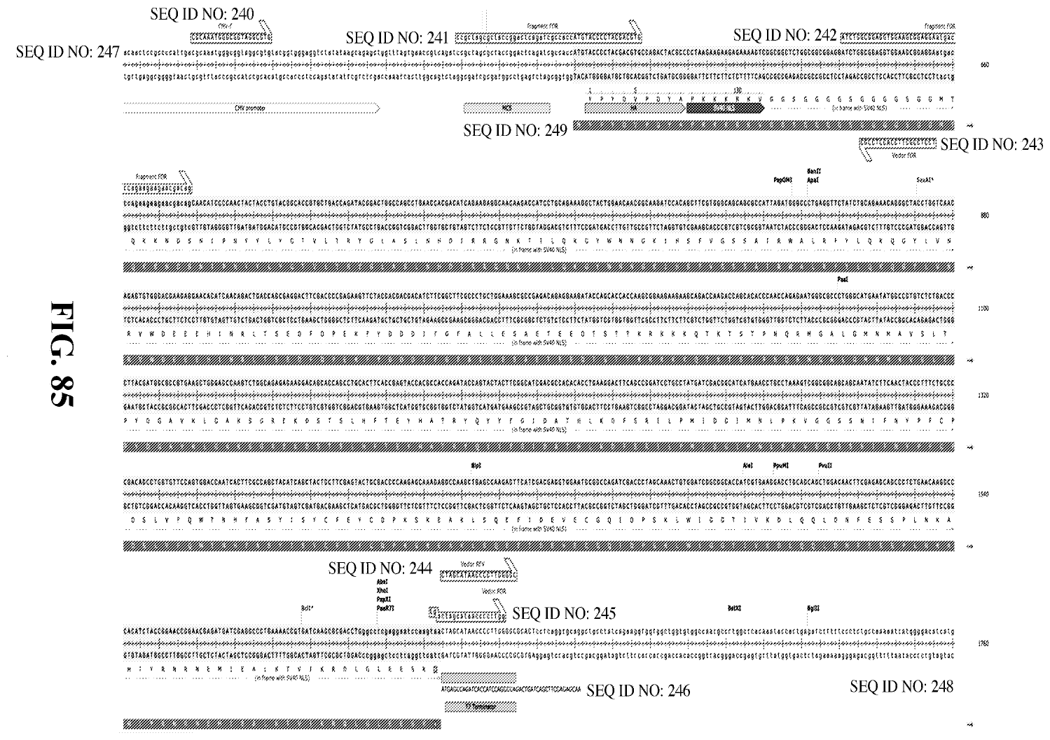

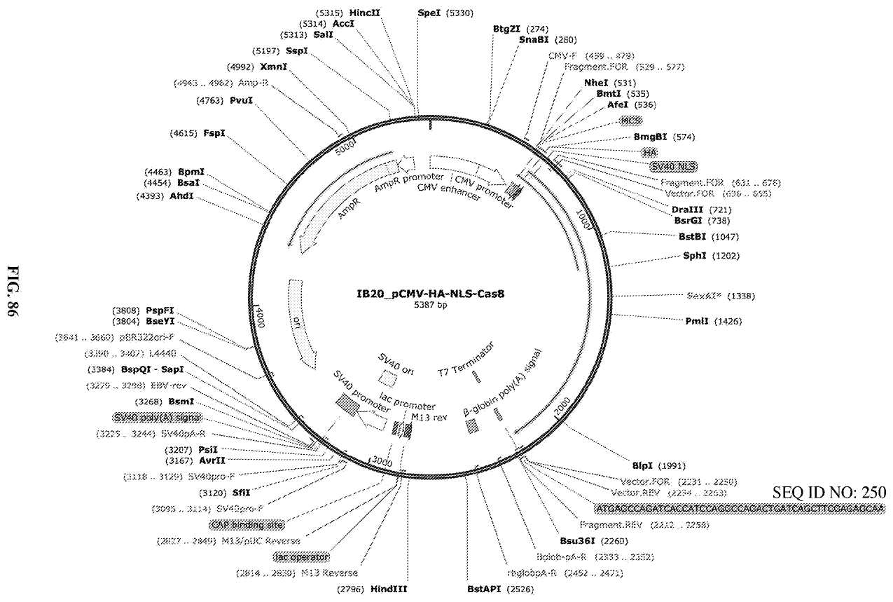

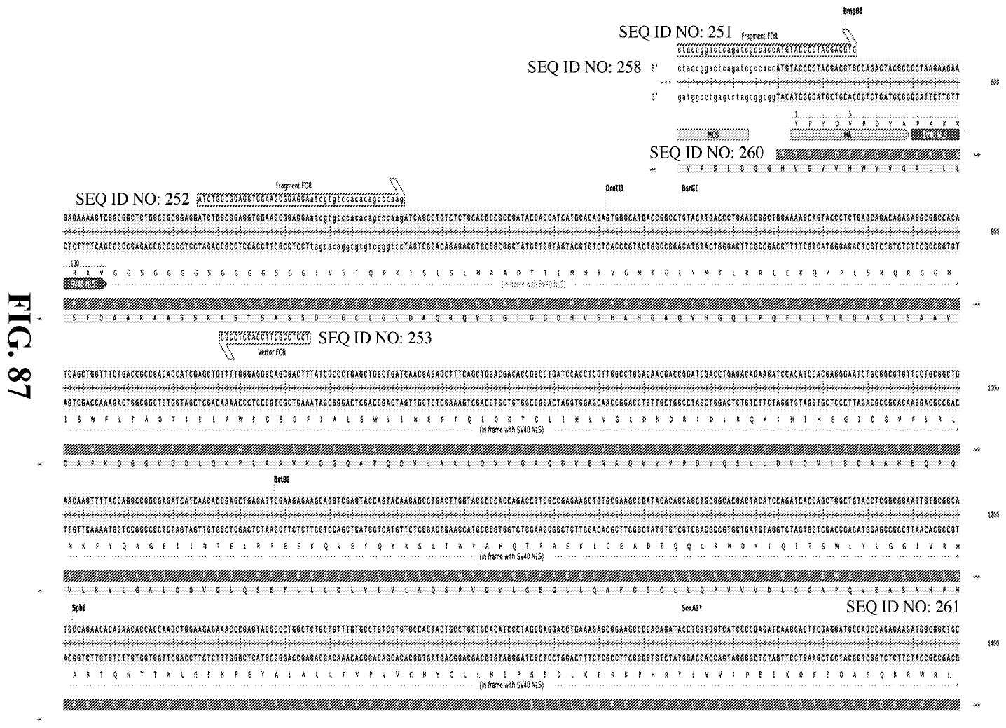

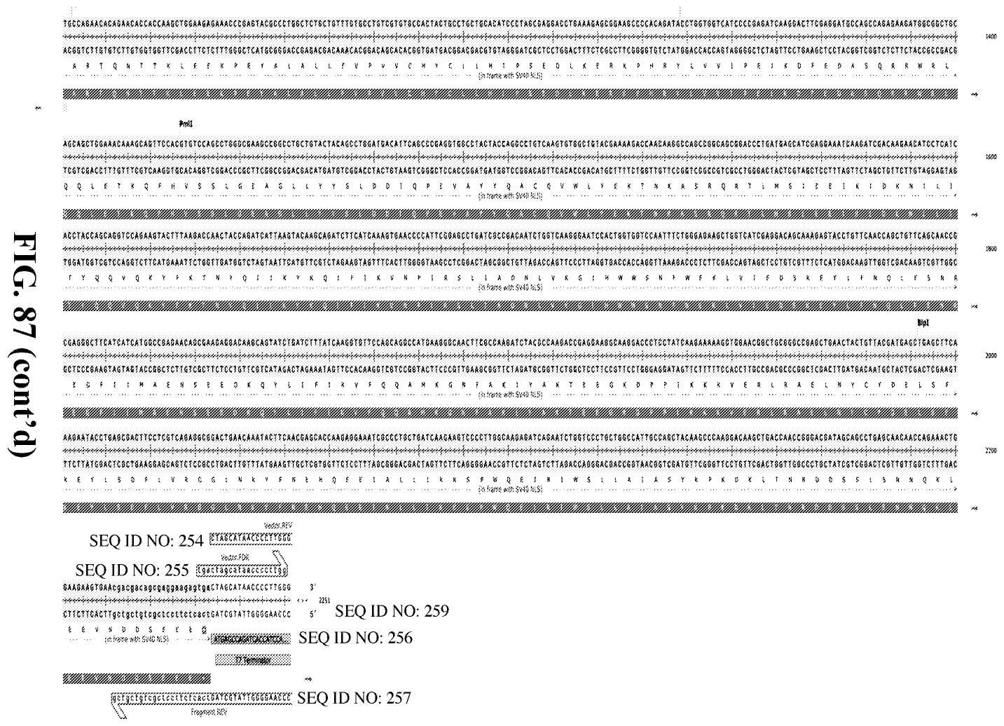

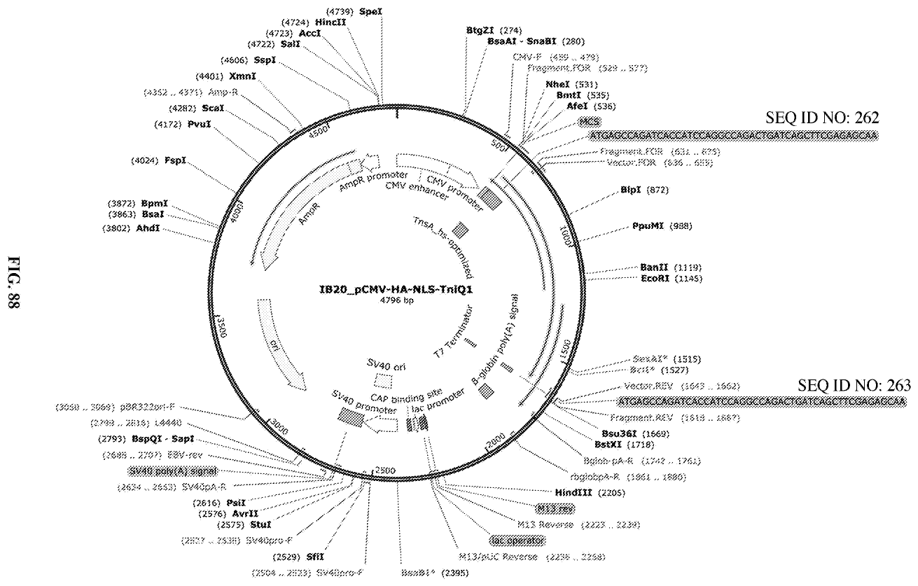

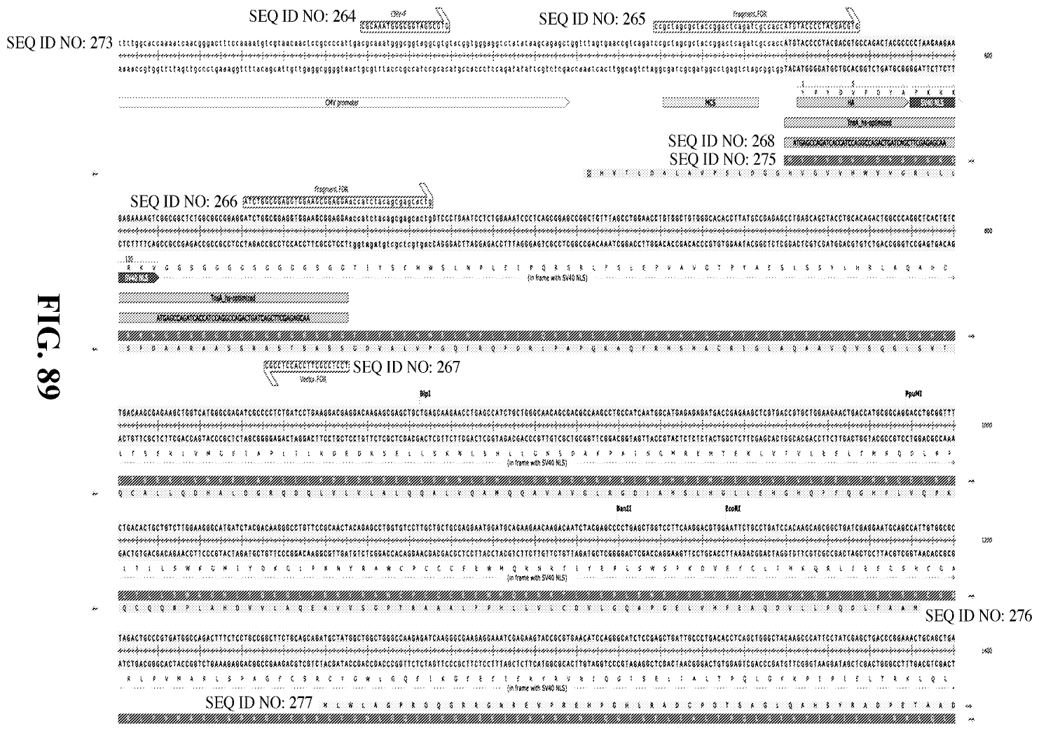

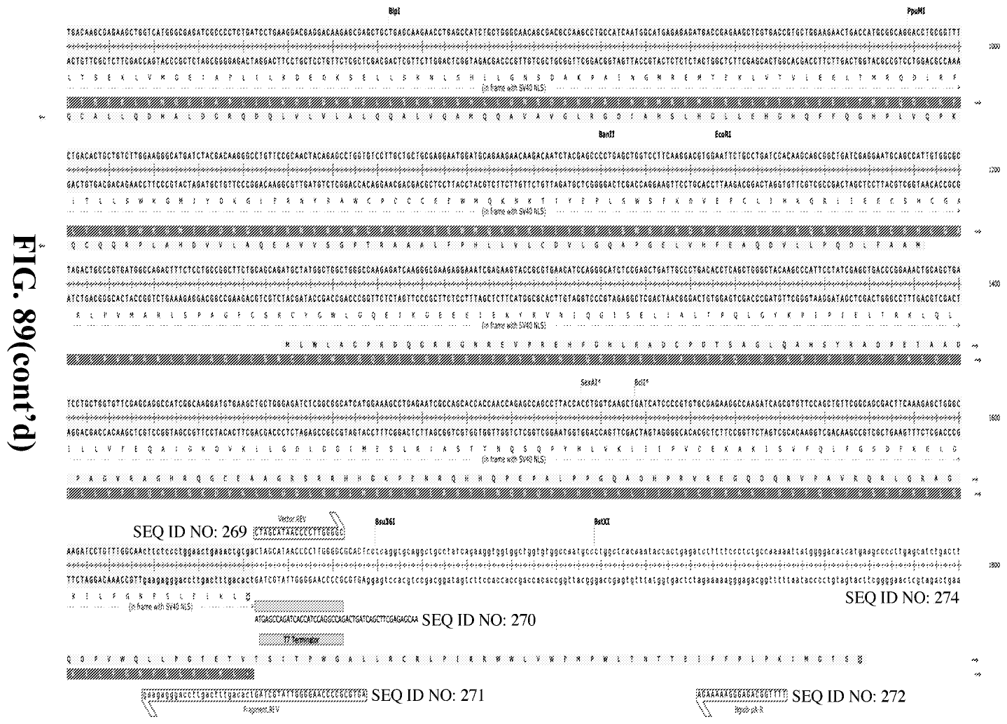

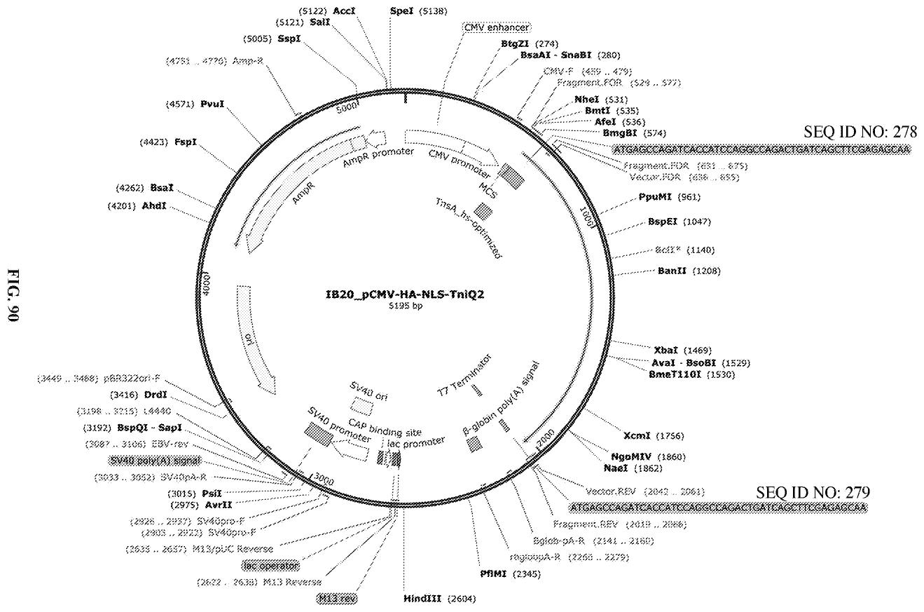

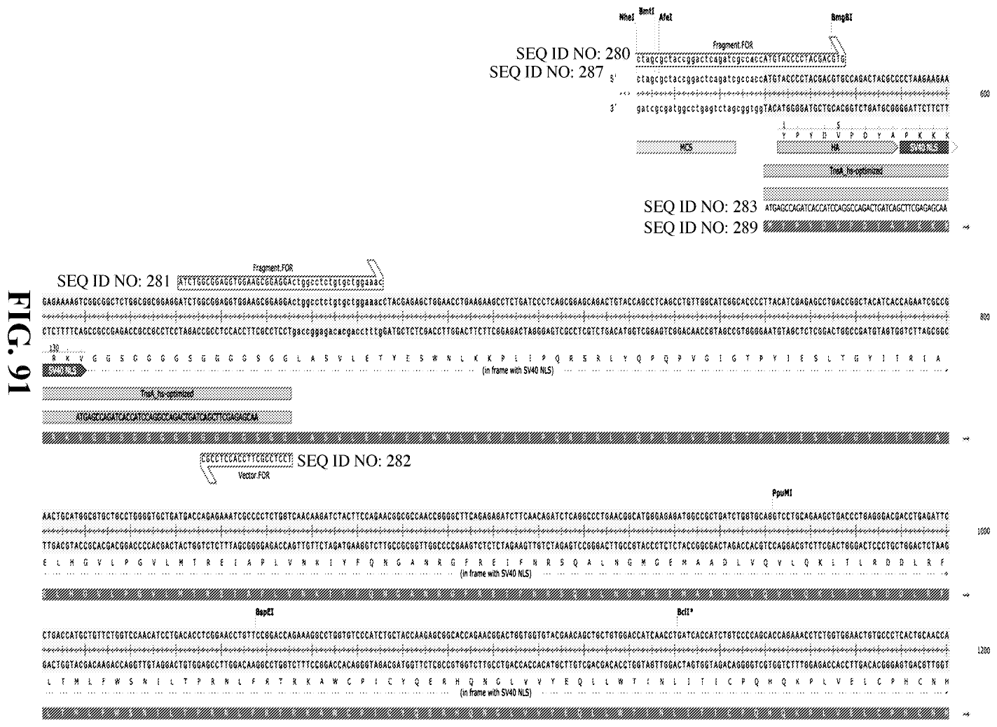

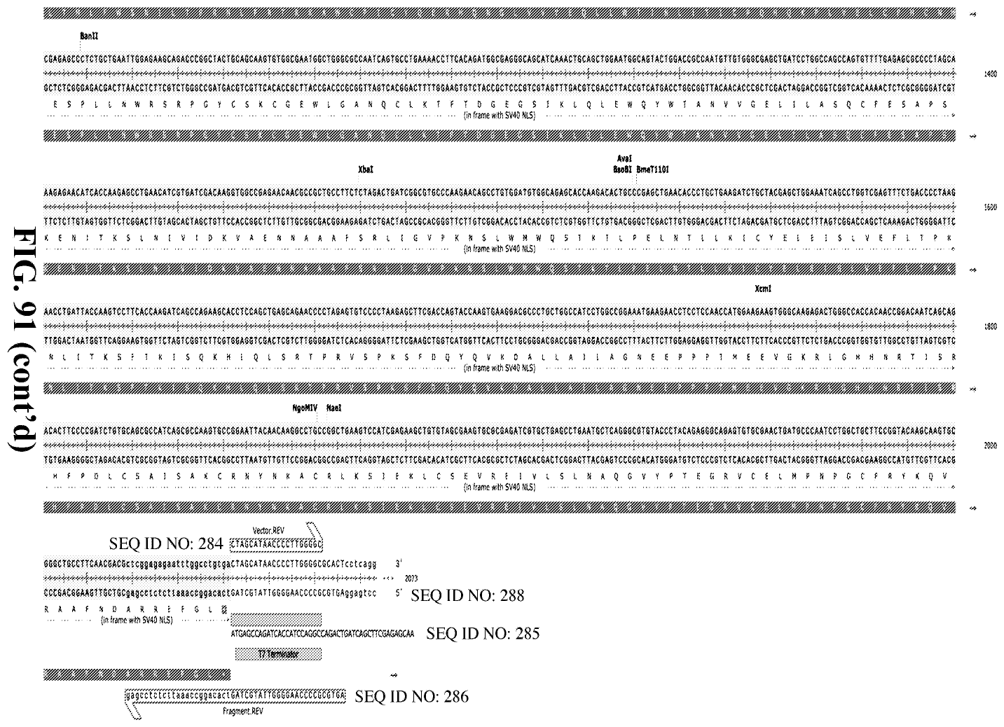

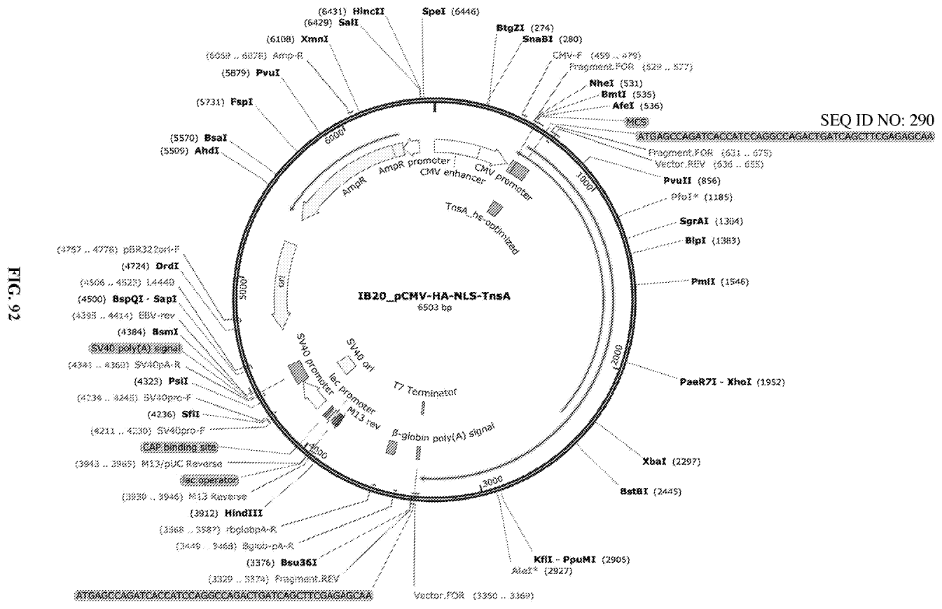

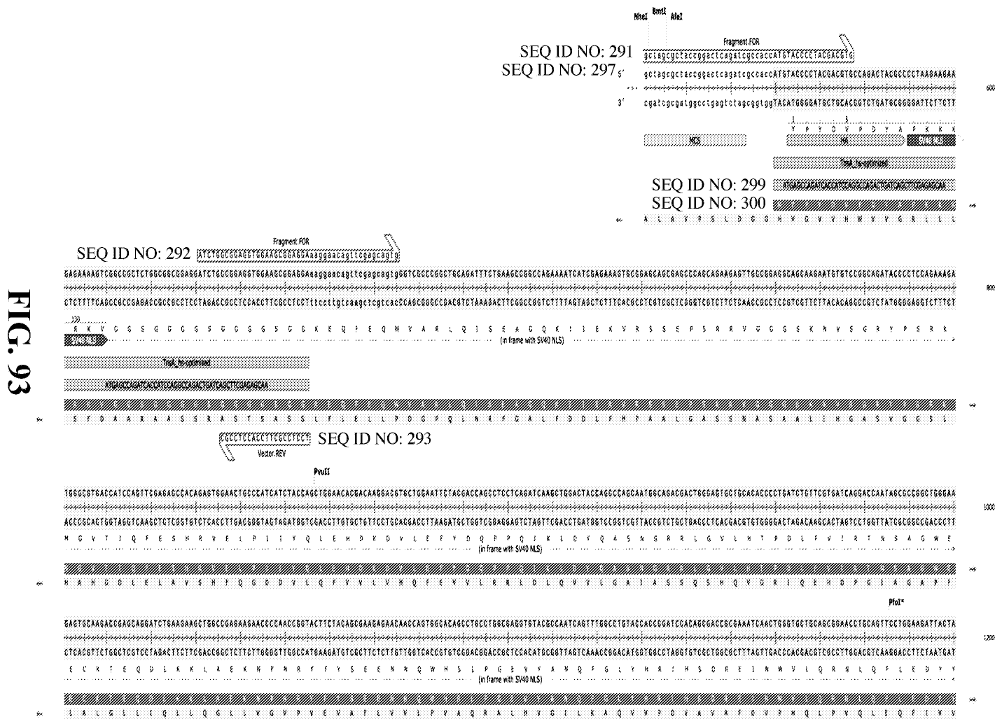

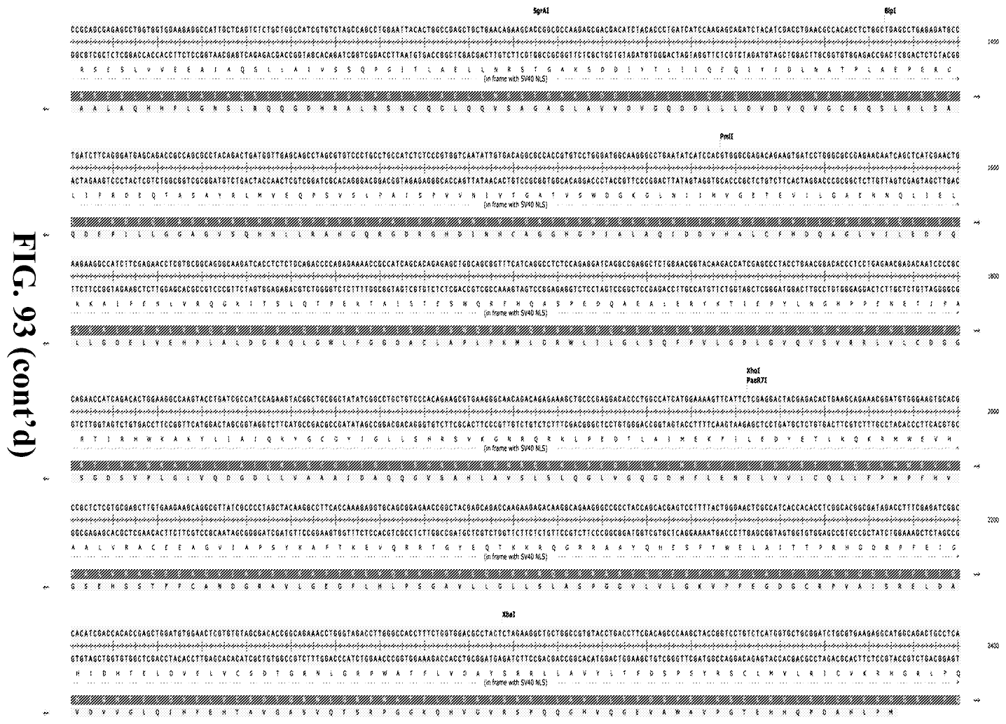

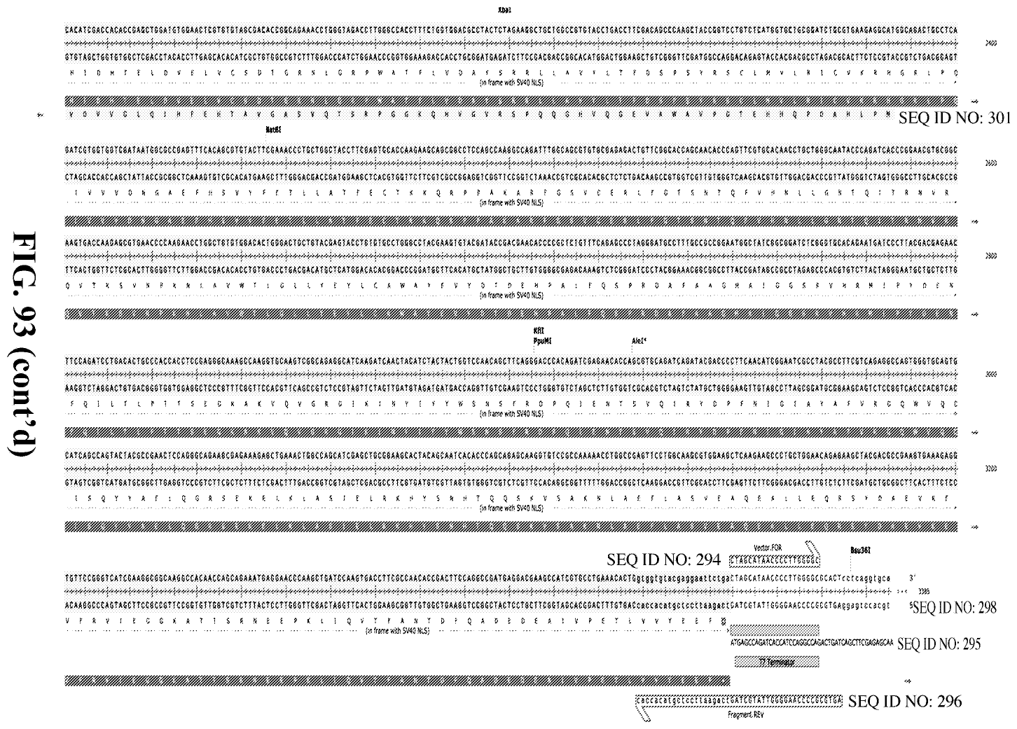

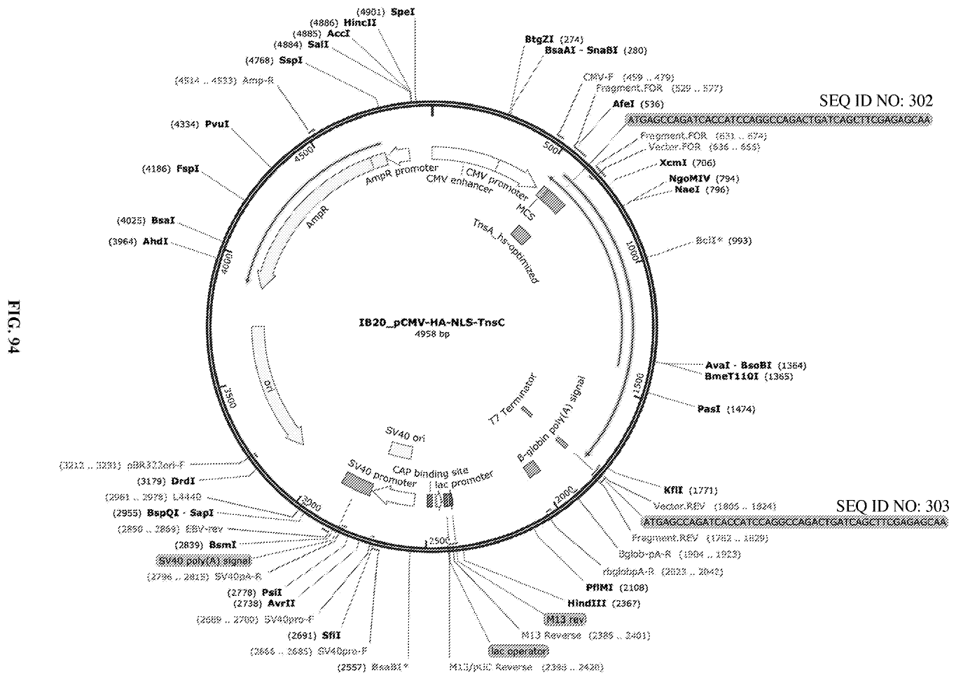

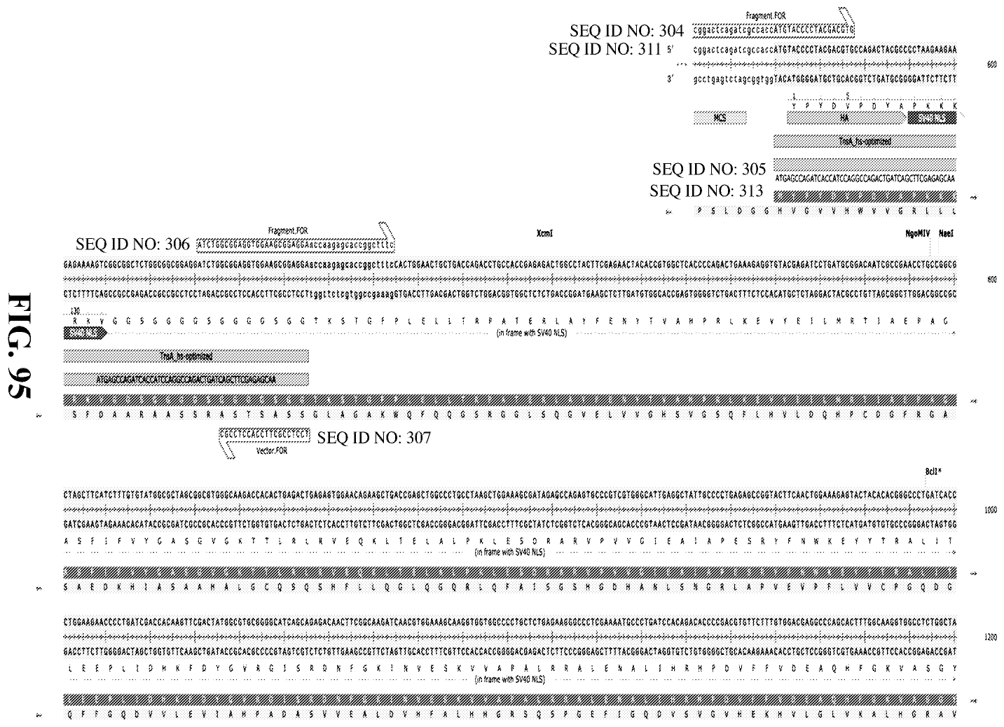

TABLE 9

| | Plasmid | | Expression |
|---|---|---|---|
| Plasmids | Maps | Expression cassette sequences | cassette Maps |
| IB20_pCMV- | FIG. 72 | Cas5-NLS-HA: | Cas5-NLS-HA: |
| Cas5-NLS-HA | | MIAPLILYLDVPFTTFRESHAREMGKTYPVPPPATVYGMLLSLVGE | FIG. 73 |
| | | TNVYRHCGVELAIAMLSSPKKSRILRQMRRFKNADFSHPENVIPC | |
| | | YQEILSNLKCLIWVRSDEEKIQPSLRERIQLAFDHPELVRRFGCLFL | |
| | | GESDQLIKTIKLAREDYLEGVRQWAIRDNRGRLTLPYWVDHVGS | |
| | | RNTRFLRYRIEEMDRLSPPDLAWTMVQSPIGGSGGGGSGGGGSGG | |
| | | PKKKRKVYPYDVPDYA* (SEQ ID NO: 83) | |
| IB20_pCMV- | FIG. 74 | Cas6-NLS-HA: | Cas6-NLS-HA: |
| Cas6-NLS-HA | | MTAILIQSEEYVDLTFKLRGAPIPLDNGYLTYAALSRICPPLHELKS | FIG. 75 |
| | | IGIHPIAGIPTRNNLLELTAQSRLKIRIYHQQIPLIYPYLAGQAFHIGQ | |
| | | NFYQLDIPDYKPLISSESVYSRLVIIKGFQDSTNFIEAVQRQMDNLG | |
| | | IQGKIELLTRQDGTPQRRQLTINKEGKQFKVRGFGVKISELNPEDS | |
| | | LTLQEQGIGGKRKMMCGIFVPATRSKEEEETGGSGGGGSGGGGSG | |
| | | GPKKKRKVYPYDVPDYA* (SEQ ID NO: 84) | |
| IB20_pCMV- | FIG. 76 | Cas7-NLS-HA: | Cas7-NLS-HA: |
| Cas7-NLS-HA | | MMTQKKNDSNIPNYYLYGTVLTRYGLASLNHDIRRGNKTILQKG | FIG. 77 |
| | | YWNNGKIHSFVGSSAIRWALRFYLQKQGYLVNRVWDEEEHINRL | |
| | | TSEDFDPEKFYDDDIFGFALLESAETEEDTSTTKRKKKQTKTSTPN | |
| | | QRMGALGMNMAVSLTPYDGAVKLGAKSGREKDSTSLHFTEYHA | |
| | | TRYQYYFGIDATHLKDFSRILPMIDGIMNLPKVGGSSNIFNYPFCPD | |
| | | SLVFQWTNHFASYISYCFEYCDPKSKEAKLSQEFIDEVECGQIDPS | |
| | | KLWIGGTIVKDLQQLDNFESSPLNKAHIYRNRNEMIEALKTVIKRD | |
| | | LGLEESKGGSGGGGSGGGGSGGPKKKRKVYPYDVPDYA* (SEQ | |
| | | ID NO: 85) | |
| IB20_pCMV- | FIG. 78 | Cas8-NLS-HA: | Cas8-NLS-HA: |
| Cas8-NLS-HA | | MIVSTQPKISLSLHAADTTIMHRVGMTGLYMTLKRLEKQYPLSRQ | FIG. 79 |
| | | RGGHISWFLTADTIELFWEGSDFIALSWLINESFQLDDTGLIHLVGL | |
| | | DNDRIDLRQKIHIHEGICGVFLRLNKFYQAGEIINTELRFEEKQVEY | |
| | | QYKSLTWYAHQTFAEKLCEADTQQLRHDYIQITSWLYLGGIVRH | |
| | | ARTQNTTKLEEKPEYALALLFVPVVCHYCLLHIPSEDLKERKPHR | |
| | | YLVVIPEIKDFEDASQRRWRLQQLETKQFHVSSLGEAGLLYYSLD | |
| | | DIQPEVAYYQACQVWLYEKTNKASRQRTLMSIEEIKIDKNILITYQ | |
| | | QVQKYFKTNYQIIKYKQIFIKVNPIRSLIADNLVKGIHWWSNFWEK | |
| | | LVIEDSKEYLFNQLFSNREGFIIMAENSEEDKQYLIFIKVFQQAMK | |
| | | GNFAKIYAKTEEGKDPPIKKKVERLRAELNYCYDELSFKEYLSDFL | |
| | | VRGGLNKYFNEHQEEIALLIKKSPWQEIRIWSLLAIASYKPKDKLT | |
| | | NRDDSSLSNNQKLEEVNDDSEEEGGSGGGGSGGGGSGGPKKKRK | |
| | | VYPYDVPDYA* (SEQ ID NO: 86) | |
| IB20_pCMV- | FIG. 80 | HA-NLS-Cas5: | HA-NLS-Cas5: |
| HA-NLS-Cas5 | | MYPYDVPDYAPKKKRKVGGSGGGGSGGGGSGGIAPLILYLDVPF | FIG. 81 |
| | | TTFRESHAREMGKTYPVPPPATVYGMLLSLVGETNVYRHCGVEL | |
| | | AIAMLSSPKKSRILRQMRRFKNADFSHPENVIPCYQEILSNLKCLIW | |
| | | VRSDEEKIQPSLRERIQLAFDHPELVRRFGCLFLGESDQLIKTIKLA | |
| | | REDYLEGVRQWAIRDNRGRLTLPYWVDHVGSRNTRFLRYRIEEM | |
| | | DRLSPPDLAWTMVQSPI* (SEQ ID NO: 87) | |
| IB20_pCMV- | FIG. 82 | HA-NLS-Cas6: | HA-NLS-Cas6: |
| HA-NLS-Cas6 | | MYPYDVPDYAPKKKRKVGGSGGGGSGGGGSGGTAILIQSEEYVD | FIG. 83 |
| | | LTFKLRGAPIPLDNGYLTYAALSRICPPLHELKSIGIHPIAGIPTRNN | |
| | | LLELTAQSRLKIRIYHQQIPLIYPYLAGQAFHIGQNFYQLDIPDYKP | |
| | | LISSESVYSRLVIIKGFQDSTNFIEAVQRQMDNLGIQGKIELLTRQD | |
| | | GTPQRRQLTINKEGKQFKVRGFGVKISELNPEDSLTLQEQGIGGKR | |
| | | KMMCGIFVPATRSKEEEET* (SEQ ID NO: 88) | |
| IB20_pCMV- | FIG. 84 | HA-NLS-Cas7: | HA-NLS-Cas7: |
| HA-NLS-Cas7 | | MYPYDVPDYAPKKKRKVGGSGGGGSGGGGSGGMTQKKNDSNIP | FIG. 85 |
| | | NYYLYGTVLTRYGLASLNHDIRRGNKTILQKGYWNNGKIHSFVG | |
| | | SSAIRWALRFYLQKQGYLVNRVWDEEEHINRLTSEDFDPEKFYDD | |
| | | DIFGFALLESAETEEDTSTTKRKKKQTKTSTPNQRMGALGMNMA | |
| | | VSLTPYDGAVKLGAKSGREKDSTSLHFTEYHATRYQYYFGIDATH | |
| | | LKDFSRILPMIDGIMNLPKVGGSSNIFNYPFCPDSLVFQWTNHFAS | |
| | | YISYCFEYCDPKSKEAKLSQEFIDEVECGQIDPSKLWIGGTIVKDLQ | |
| | | QLDNFESSPLNKAHIYRNRNEMIEALKTVIKRDLGLEESK* (SEQ | |
| | | ID NO: 89) | |
| IB20_pCMV- | FIG. 86 | HA-NLS-Cas8: | HA-NLS-Cas8: |
| HA-NLS-Cas8 | | MYPYDVPDYAPKKKRKVGGSGGGGSGGGGSGGIVSTQPKISLSL | FIG. 87 |
| | | HAADTTIMHRVGMTGLYMTLKRLEKQYPLSRQRGGHISWFLTAD | |
| | | TIELFWEGSDFIALSWLINESFQLDDTGLIHLVGLDNDRIDLRQKIH | |
| | | IHEGICGVFLRLNKFYQAGEIINTELRFEEKQVEYQYKSLTWYAHQ | |
| | | TFAEKLCEADTQQLRHDYIQITSWLYLGGIVRHARTQNTTKLEEK | |
| | | PEYALALLFVPVVCHYCLLHIPSEDLKERKPHRYLVVIPEIKDFED | |
| | | ASQRRWRLQQLETKQFHVSSLGEAGLLYYSLDDIQPEVAYYQAC | |
| | | QVWLYEKTNKASRQRTLMSIEEIKIDKNILITYQQVQKYFKTNYQI | |
| | | IKYKQIFIKVNPIRSLIADNLVKGIHWWSNFWEKLVIEDSKEYLFN | |
| | | QLFSNREGFIIMAENSEEDKQYLIFIKVFQQAMKGNFAKIYAKTEE | |
| | | GKDPPIKKKVERLRAELNYCYDELSFKEYLSDFLVRGGLNKYFNE | |
| | | HQEEIALLIKKSPWQEIRIWSLLAIASYKPKDKLTNRDDSSLSNNQ | |
| | | KLEEVNDDSEEE* (SEQ ID NO: 90) | |
| IB20_pCMV- | FIG. 88 | HA-NLS-TniQ1: | HA-NLS- |
| HA-NLS-TniQ1 | | MYPYDVPDYAPKKKRKVGGSGGGGSGGGGSGGTIYSEHWSLNPL | TniQ1: |
| | | EIPQRSRLFSLEPVAVGTPYAESLSSYLHRLAQAHCLTSEKLVMGE | FIG. 89 |
| | | IAPLILKDEDKSELLSKNLSHLLGNSDAKPAINGMREMTEKLVTVL | |
| | | EELTMRQDLRFLTLLSWKGMIYDKGLFRNYRAWCPCCCEEWMQ | |
| | | KNKTIYEPLSWSFKDVEFCLIHKQRLIEECSHCGARLPVMARLSPA | |
| | | GFCSRCYGWLGQEIKGEEEIEKYRVNIQGISELIALTPQLGYKPIPIE | |
| | | LTRKLQLILLVFEQAIGKDVKLLGDLGGIMESLRIASTTNQSQPYH | |
| | | LVKLIIPVCEKAKISVFQLFGSDFKELGKILFGNFSLELKL* (SEQ ID | |
| | | NO: 91) | |
| IB20_pCMV- | FIG. 90 | HA-NLS-TniQ2 | HA-NLS- |
| HA-NLS-TniQ2 | | MYPYDVPDYAPKKKRKVGGSGGGGSGGGGSGGLASVLETYESW | TniQ2: |
| | | NLKKPLIPQRSRLYQPQPVGIGTPYIESLTGYITRIAELHGVLPGVL | FIG. 91 |
| | | MTREIAPLVNKIYFQNGANRGFREIFNRSQALNGMGEMAADLVQ | |
| | | VLQKLTLRDDLRFLTMLFWSNILTPRNLFRTRKAWCPICYQERHQ | |
| | | NGLVVYEQLLWTINLITICPQHQKPLVELCPHCNHESPLLNWRSRP | |
| | | GYCSKCGEWLGANQCLKTFTDGEGSIKLQLEWQYWTANVVGELI | |
| | | LASQCFESAPSKENITKSLNIVIDKVAENNAAAFSRLIGVPKNSLW | |
| | | MWQSTKTLPELNTLLKICYELEISLVEFLTPKNLITKSFTKISQKHL | |
| | | QLSRTPRVSPKSFDQYQVKDALLAILAGNEEPPPTMEEVGKRLGH | |
| | | HNRTISRHFPDLCSAISAKCRNYNKACRLKSIEKLCSEVREIVLSLN | |
| | | AQGVYPTEGRVCELMPNPGCFRYKQVRAAFNDARREFGL* (SEQ | |
| | | ID NO: 92) | |
| IB20_pCMV- | FIG. 92 | HA-NLS-TnsA | HA-NLS-TnsA: |
| HA-NLS-TnsA | | MYPYDVPDYAPKKKRKVGGSGGGGSGGGGSGGKEQFEQWVAR | FIG. 93 |
| | | LQISEAGQKIIEKVRSSEPSRRVGGGSKNVSGRYPSRKMGVTIQFES | |
| | | HRVELPIIYQLEHDKDVLEFYDQPPQIKLDYQASNGRRLGVLHTPD | |
| | | LFVIRTNSAGWEECKTEQDLKKLAEKNPNRYFYSEENNQWHSLP | |
| | | GEVYANQFGLYHRIHSDREINWVLQRNLQFLEDYYRSESLVVEEA | |
| | | IAQSLLAIVSSQPGITLAELLNRSTGAKSDDIYTLIIQEQIYIDLNATP | |
| | | LAEPERCLIFRDEQTASAYRLMVEQPSVSLPAISPVVNIVTGATVS | |
| | | WDGKGLNIIHVGETEVILGAENNQLIELKKAIFENLVRQGKITSLQ | |
| | | TPEKTAISTESWQRFHQASPEDQAEALERYKTIEPYLNGHPPENETI | |
| | | PARTIRHWKAKYLIAIQKYGCGYIGLLSHRSVKGNRQRKLPEDTL | |
| | | AIMEKFILEDYETLKQKRMWEVHAALVRACEEAGVIAPSYKAFT | |
| | | KEVQRRTGYEQTKKRQGRRAAYQHESFYWELAITTPRHGDRPFEI | |
| | | GHIDHTELDVELVCSDTGRNLGRPWATFLVDAYSRRLLAVYLTFD | |
| | | SPSYRSCLMVLRICVKRHGRLPQIVVVDNGAEFHSVYFETLLATFE | |
| | | CTKKQRPPAKARFGSVCERLFGTSNTQFVHNLLGNTQITRNVRQV | |
| | | TKSVNPKNLAVWTLGLLYEYLCAWAYEVYDTDEHPALFQSPRDA | |
| | | FAAGMAIGGSRVHRMIPYDENFQILTLPTTSEGKAKVQVGRGIKIN | |
| | | YIYYWSNSFRDPQIENTSVQIRYDPFNIGIAYAFVRGQWVQCISQY | |
| | | YAELQGRSEKELKLASIELRKHYSNHTQQSKVSAKNLAEFLASVE | |
| | | AQEALLEQRSYDAEVKEVFRVIEGGKATTSRNEEPKLIQVTFANT | |
| | | DFQADEDEAIVPETLVVYEEF* (SEQ ID NO: 93) | |
| IB20_pCMV- | FIG. 94 | HA-NLS-TnsC | HA-NLS-TnsC: |
| HA-NLS-TnsC | | MYPYDVPDYAPKKKRKVGGSGGGGSGGGGSGGTKSTGFPLELLT | FIG. 95 |
| | | RPATERLAYFENYTVAHPRLKEVYEILMRTIAEPAGASFIFVYGAS | |
| | | GVGKTTLRLRVEQKLTELALPKLESDRARVPVVGIEAIAPESRYFN | |
| | | WKEYYTRALITLEEPLIDHKFDYGVRGISRDNFGKINVESKVVAPA | |
| | | LRRALENALIHRHPDVFFVDEAQHFGKVASGYKLQDQLDCLKSL | |
| | | ANMTGILHCLLGTYELLTFRNLSGQLSRRSVDIHFRRYCADSPEDV | |
| | | QAFKSVLLTFQQHLPLAETPNLVDHWEYFYERTLGCIGTLKDWLK | |
| | | RVLSDALDREATTITLKDLQKRALSVAQCQKMFKEIQEGERQLSE | |
| | | TEADVQNLRSALGLGAKPIVLPEETPKTTRPPGKVGKRKPKRDPIG | |
| | | VQQDVS* (SEQ ID NO: 94) | |
| IB20_pCMV- | FIG. 96 | TniQ1-NLS-HA | TniQ1-NLS-HA |
| TniQ1-NLS-HA | | MTIYSEHWSLNPLEIPQRSRLFSLEPVAVGTPYAESLSSYLHRLAQ | FIG. 97 |
| | | AHCLTSEKLVMGEIAPLILKDEDKSELLSKNLSHLLGNSDAKPAIN | |
| | | GMREMTEKLVTVLEELTMRQDLRFLTLLSWKGMIYDKGLFRNYR | |
| | | AWCPCCCEEWMQKNKTIYEPLSWSFKDVEFCLIHKQRLIEECSHC | |
| | | GARLPVMARLSPAGFCSRCYGWLGQEIKGEEEIEKYRVNIQGISEL | |
| | | IALTPQLGYKPIPIELTRKLQLILLVFEQAIGKDVKLLGDLGGIMES | |
| | | LRIASTTNQSQPYHLVKLIIPVCEKAKISVFQLFGSDFKELGKILFG | |
| | | NFSLELKLGGSGGGGSGGGGSGGPKKKRKVYPYDVPDYA* (SEQ | |
| | | ID NO: 95) | |
| IB20_pCMV- | FIG. 98 | TniQ2-NLS-HA: | TniQ2-NLS-HA |
| TniQ2-NLS-HA | | MLASVLETYESWNLKKPLIPQRSRLYQPQPVGIGTPYIESLTGYITR | FIG. 99 |
| | | IAELHGVLPGVLMTREIAPLVNKIYFQNGANRGFREIFNRSQALNG | |
| | | MGEMAADLVQVLQKLTLRDDLRFLTMLFWSNILTPRNLFRTRKA | |
| | | WCPICYQERHQNGLVVYEQLLWTINLITICPQHQKPLVELCPHCN | |
| | | HESPLLNWRSRPGYCSKCGEWLGANQCLKTFTDGEGSIKLQLEW | |
| | | QYWTANVVGELILASQCFESAPSKENITKSLNIVIDKVAENNAAAF | |
| | | SRLIGVPKNSLWMWQSTKTLPELNTLLKICYELEISLVEFLTPKNLI | |
| | | TKSFTKISQKHLQLSRTPRVSPKSFDQYQVKDALLAILAGNEEPPP | |
| | | TMEEVGKRLGHHNRTISRHFPDLCSAISAKCRNYNKACRLKSIEKL | |
| | | CSEVREIVLSLNAQGVYPTEGRVCELMPNPGCFRYKQVRAAFND | |
| | | ARREFGLGGSGGGGSGGGGSGGPKKKRKVYPYDVPDYA* (SEQ | |
| | | ID NO: 96) | |
| IB20_pCMV- | FIG. 100 | TnsA-NLS-HA | TnsA-NLS-HA: |
| TnsA-NLS-HA | | MKEQFEQWVARLQISEAGQKIIEKVRSSEPSRRVGGGSKNVSGRY | FIG. 101 |
| | | PSRKMGVTIQFESHRVELPIIYQLEHDKDVLEFYDQPPQIKLDYQA | |
| | | SNGRRLGVLHTPDLFVIRTNSAGWEECKTEQDLKKLAEKNPNRYF | |
| | | YSEENNQWHSLPGEVYANQFGLYHRIHSDREINWVLQRNLQFLE | |
| | | DYYRSESLVVEEAIAQSLLAIVSSQPGITLAELLNRSTGAKSDDIYT | |
| | | LIIQEQIYIDLNATPLAEPERCLIFRDEQTASAYRLMVEQPSVSLPAI | |
| | | SPVVNIVTGATVSWDGKGLNIIHVGETEVILGAENNQLIELKKAIF | |
| | | ENLVRQGKITSLQTPEKTAISTESWQRFHQASPEDQAEALERYKTI | |
| | | EPYLNGHPPENETIPARTIRHWKAKYLIAIQKYGCGYIGLLSHRSV | |
| | | KGNRQRKLPEDTLAIMEKFILEDYETLKQKRMWEVHAALVRACE | |
| | | EAGVIAPSYKAFTKEVQRRTGYEQTKKRQGRRAAYQHESFYWEL | |
| | | AITTPRHGDRPFEIGHIDHTELDVELVCSDTGRNLGRPWATFLVDA | |
| | | YSRRLLAVYLTFDSPSYRSCLMVLRICVKRHGRLPQIVVVDNGAE | |
| | | FHSVYFETLLATFECTKKQRPPAKARFGSVCERLFGTSNTQFVHNL | |
| | | LGNTQITRNVRQVTKSVNPKNLAVWTLGLLYEYLCAWAYEVYD | |
| | | TDEHPALFQSPRDAFAAGMAIGGSRVHRMIPYDENFQILTLPTTSE | |
| | | GKAKVQVGRGIKINYIYYWSNSFRDPQIENTSVQIRYDPFNIGIAY | |
| | | AFVRGQWVQCISQYYAELQGRSEKELKLASIELRKHYSNHTQQSK | |
| | | VSAKNLAEFLASVEAQEALLEQRSYDAEVKEVFRVIEGGKATTSR | |
| | | NEEPKLIQVTFANTDFQADEDEAIVPETLVVYEEFGGSGGGGSGG | |
| | | GGSGGPKKKRKVYPYDVPDYA* (SEQ ID NO: 97) | |
| IB20_pCMV- | FIG. 102 | TnsC-NLS-HA: | TnsC-NLS-HA: |
| TnsC-NLS-HA | | MTKSTGFPLELLTRPATERLAYFENYTVAHPRLKEVYEILMRTIAE | FIG. 103 |
| | | PAGASFIFVYGASGVGKTTLRLRVEQKLTELALPKLESDRARVPV | |
| | | VGIEAIAPESRYFNWKEYYTRALITLEEPLIDHKFDYGVRGISRDNF | |
| | | GKINVESKVVAPALRRALENALIHRHPDVFFVDEAQHFGKVASGY | |
| | | KLQDQLDCLKSLANMTGILHCLLGTYELLTFRNLSGQLSRRSVDI | |
| | | HFRRYCADSPEDVQAFKSVLLTFQQHLPLAETPNLVDHWEYFYER | |
| | | TLGCIGTLKDWLKRVLSDALDREATTITLKDLQKRALSVAQCQK | |
| | | MFKEIQEGERQLSETEADVQNLRSALGLGAKPIVLPEETPKTTRPP | |
| | | GKVGKRKPKRDPIGVQQDVSGGSGGGGSGGGGSGGPKKKRKVY | |
| | | PYDVPDYA* (SEQ ID NO: 98) | |

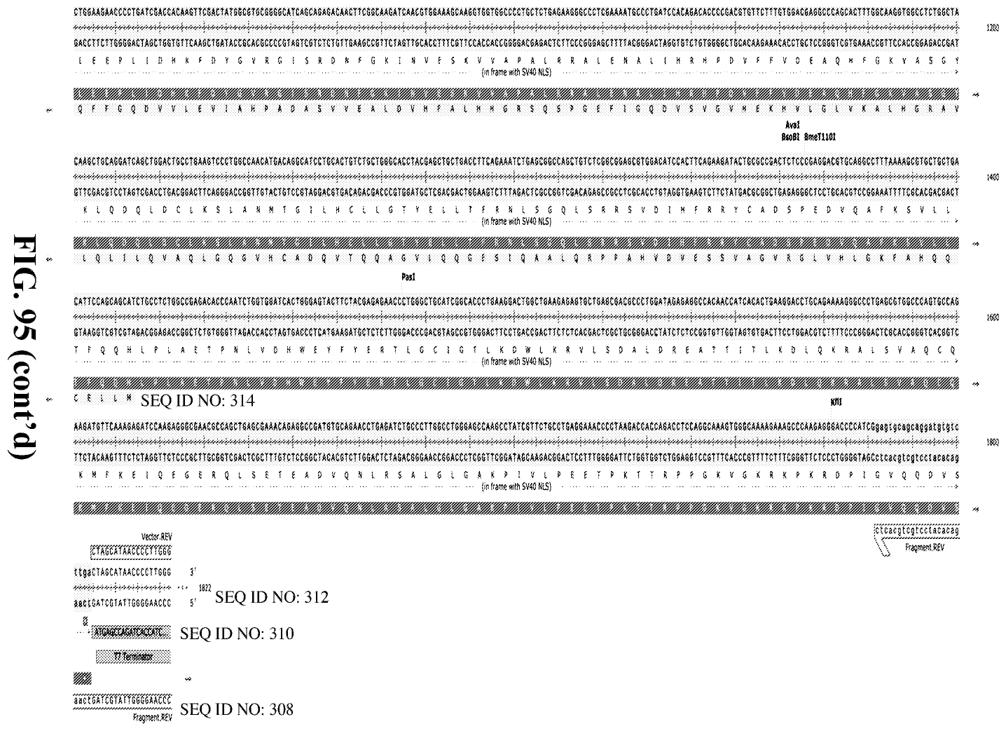

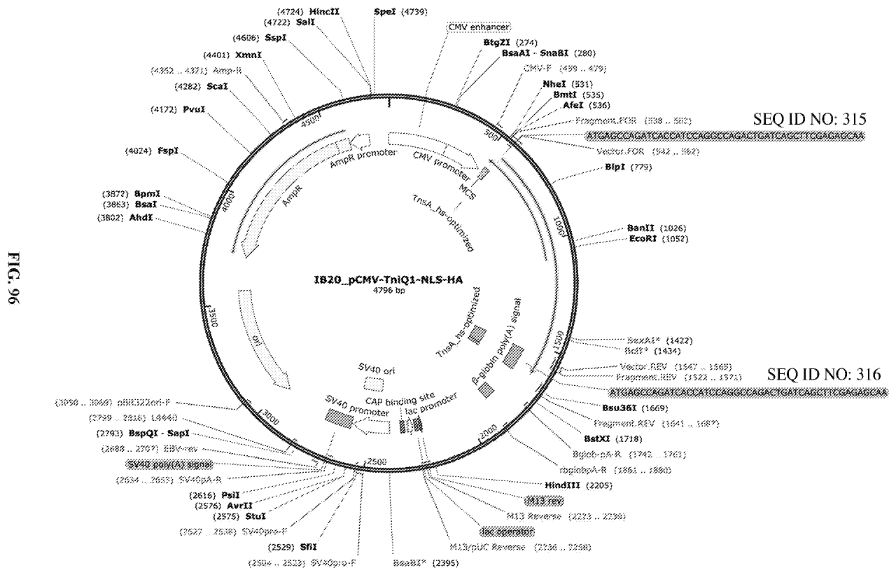

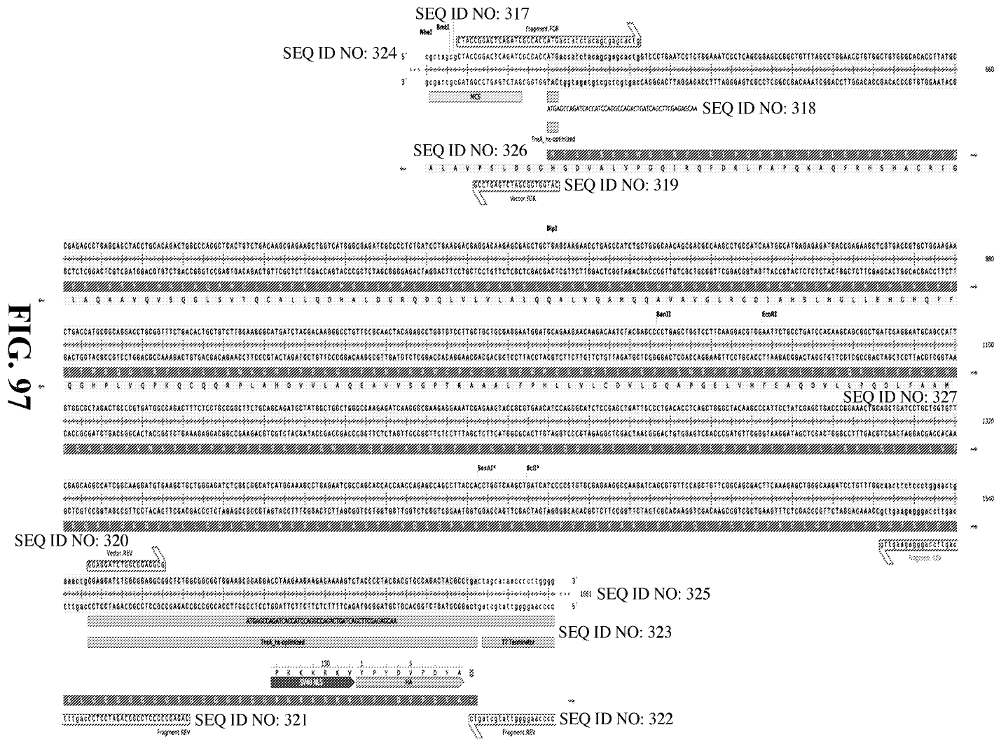

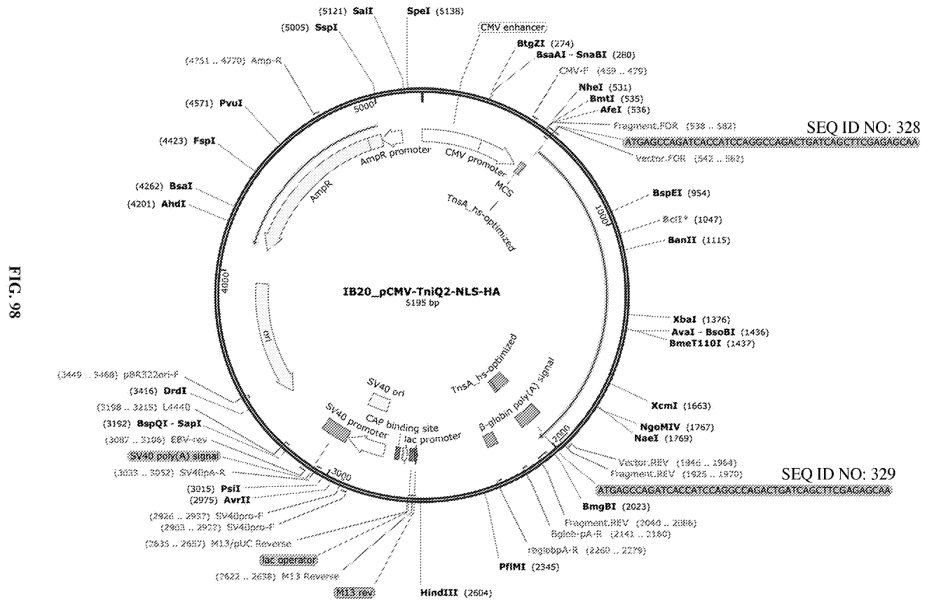

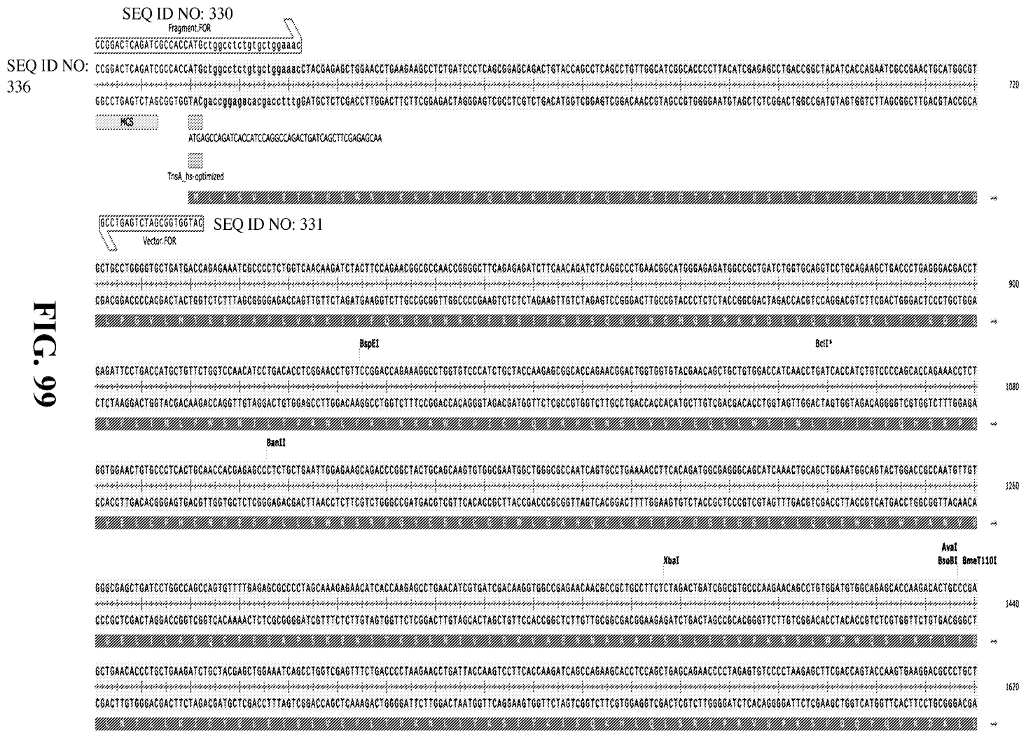

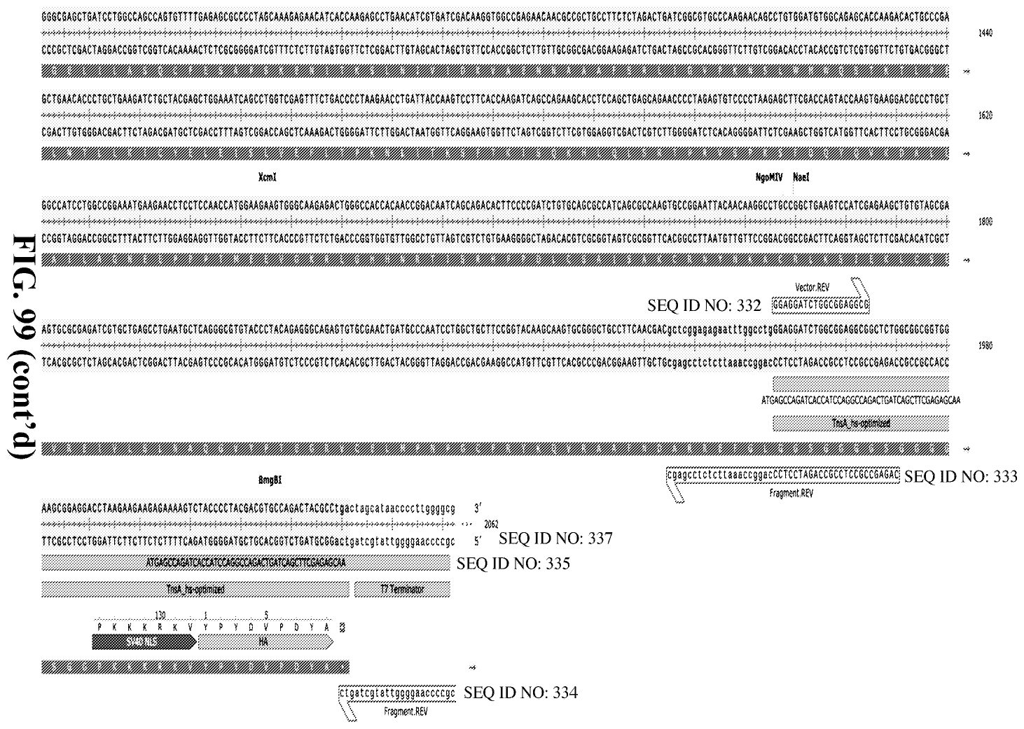

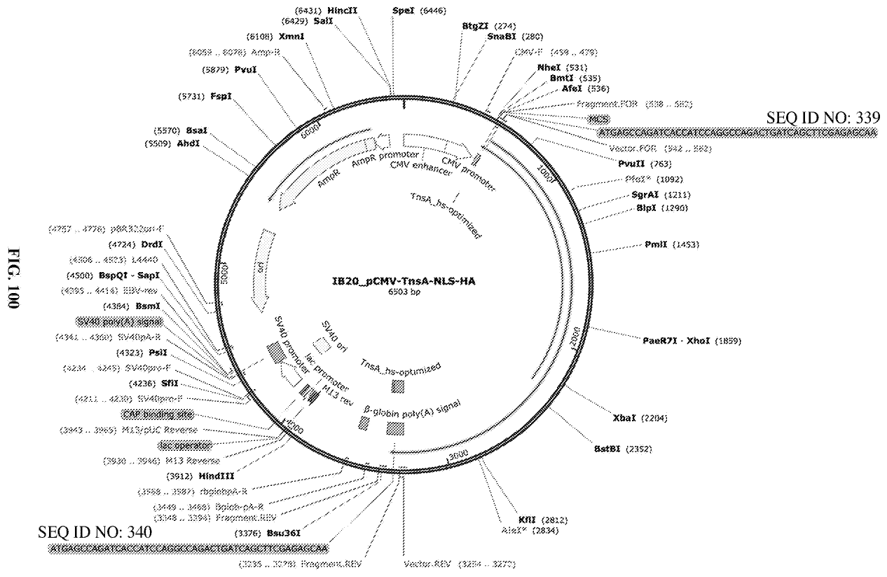

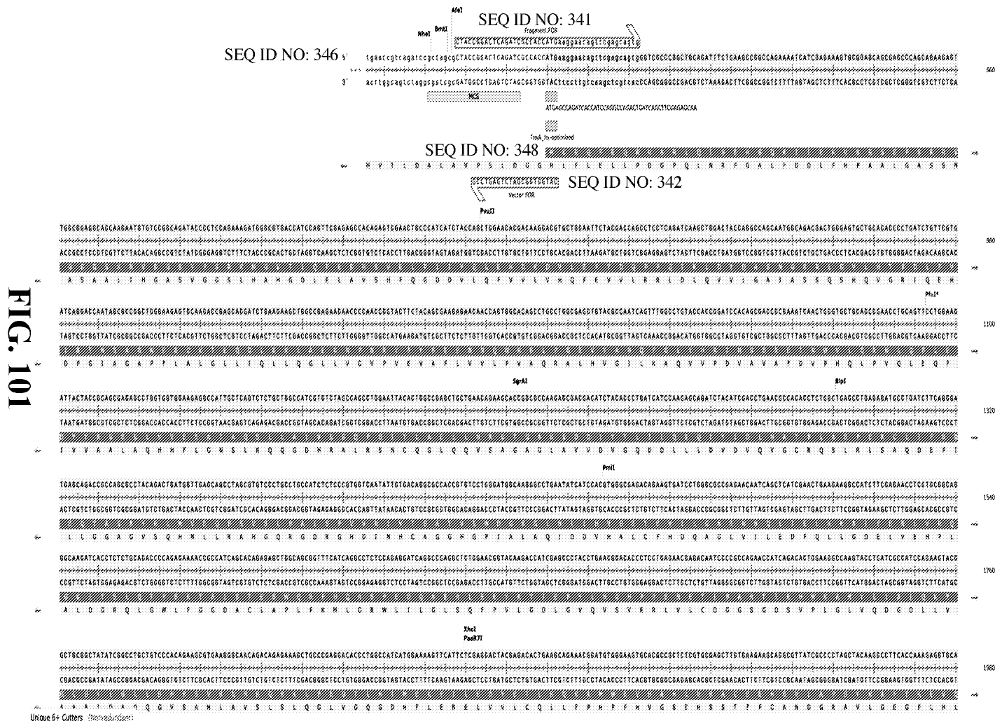

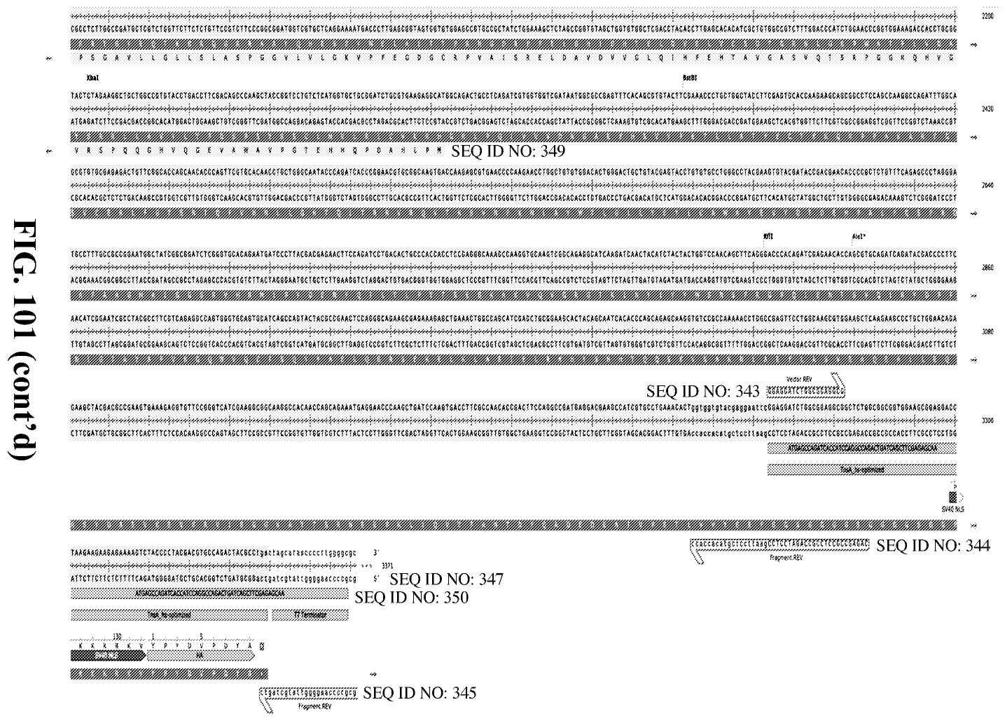

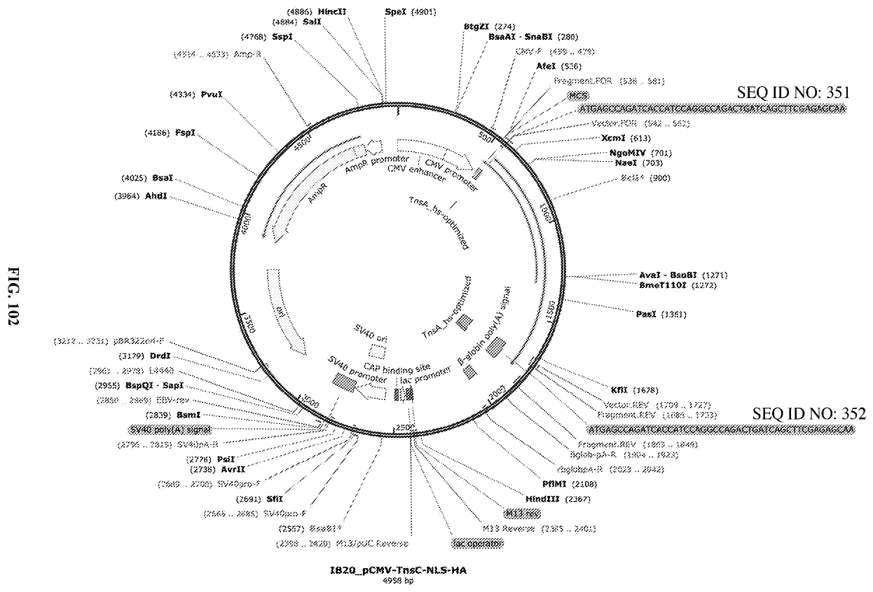

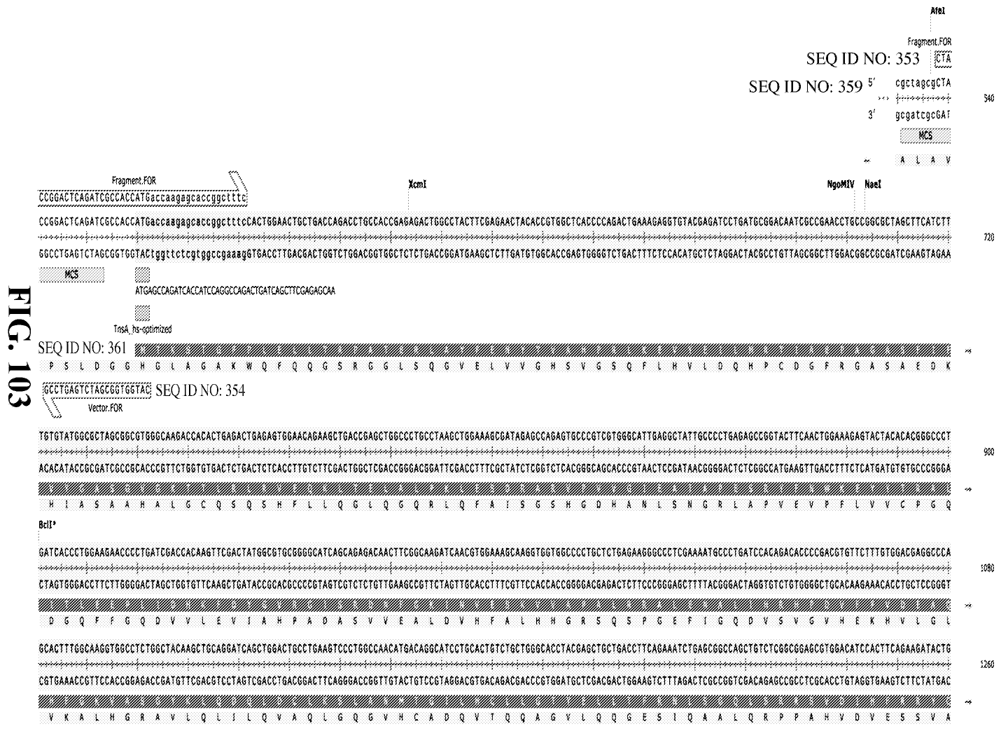

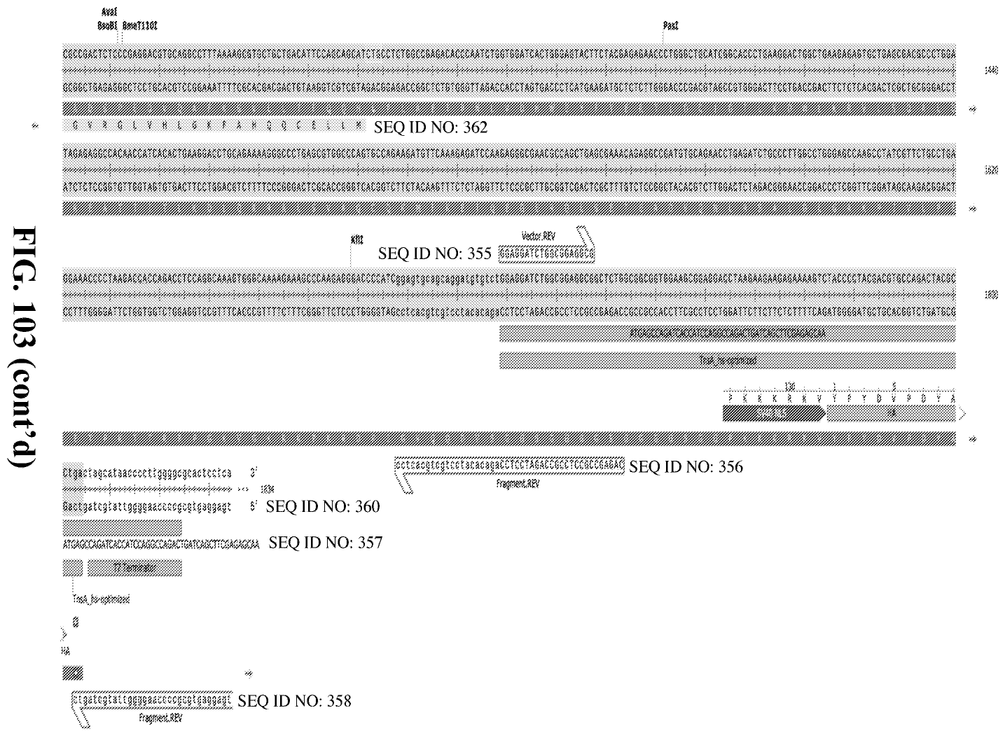

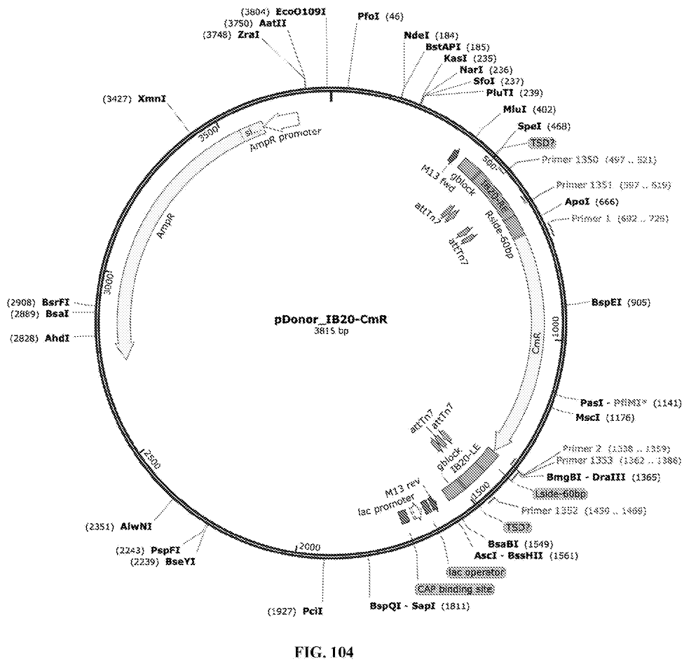

FIG. 104 shows the donor plasmid (pDonor IB20-CmR) map. The sequence with is below.

| primer_bind | 379 . . . 395 |
|---|---|
| | /label = M13 fwd |
| | /note = “common sequencing primer, one of multiple similar |
| | variants” |
| misc_feature | 416 . . . 470 |
| | /label = gblock |
| misc_feature | 471 . . . 475 |
| | /label = TSD? |
| misc_feature | 476 . . . 631 |
| | /label = IB20-RE |
| misc_feature | 484 . . . 501 |
| | /label = attTn7 |
| primer_bind | complement(497 . . . 521) |
| | /label = Primer 1350 |
| misc_feature | 507 . . . 524 |
| | /label = attTn7 |
| misc_feature | 584 . . . 601 |
| | /label = attTn7 |
| primer_bind | complement(597 . . . 619) |
| | /label = Primer 1351 |
| misc_feature | 614 . . . 631 |
| | /label = attTn7 |
| misc_feature | 632 . . . 691 |
| | /label = Rside-60 bp |
| misc_feature | 1360 . . . 1419 |
| | /label = Lside-60 bp |
| primer_bind | 1362 . . . 1386 |
| | /label = Primer 1353 |
| misc_feature | 1420 . . . 1491 |
| | /label = IB20-LE |
| misc_feature | 1420 . . . 1437 |
| | /label = attTn7 |
| misc_feature | 1447 . . . 1464 |
| | /label = attTn7 |
| primer_bind | 1450 . . . 1469 |
| | /label = Primer 1352 |
| misc_feature | complement 1466 . . . 1483) |
| | /label = attTn7 |
| misc_feature | 1492 . . . 1496 |
| | /label = TSD? |
| misc_feature | 1497 . . . 1551 |
| | /label = gblock |
| (SEQ ID NO: 99) | |
| 1 | tcgcgcgttt cggtgatgac ggtgaaaacc tctgacacat gcagctcccg gagacggtca |
| 61 | cagcttgtct gtaagcggat gccgggagca gacaagcccg tcagggcgcg tcagcgggtg |
| 121 | ttggcgggtg tcggggctgg cttaactatg cggcatcaga gcagattgta ctgagagtgc |
| 181 | accatatgcg gtgtgaaata ccgcacagat gcgtaaggag aaaataccgc atcaggcgcc |
| 241 | attcgccatt caggctgcgc aactgttggg aagggcgatc ggtgcgggcc tcttcgctat |
| 301 | tacgccagct ggcgaaaggg ggatgtgctg caaggcgatt aagttgggta acgccagggt |
| 361 | tttcccagtc acgacgttgt aaaacgacgg ccagtgaatt gacgcgtatt gggatcaatg |
| 421 | gctggtacta taattcccct ggttgctgaa gcggtaaaag tgcgtcaact agttttgtga |
| 481 | ctttacccat aactttgccg ttactggaac cataactttg ccgtcaacaa cactacaacc |
| 541 | ctcattcttg cgtcagctaa ttttggatga aattgacagt ttggaaccat aactttgccg |
| 601 | ctagtccaaa ttggaaggat aagcttgccg tgagtccaat caagcataga aagcaggaaa |
| 661 | acgccgaatt tttttagaat ggagcagaag cgtcgatgga gaaaaaaatc actggatata |
| 721 | ccaccgttga tatatcccaa tggcatcgta aagaacattt tgaggcattt cagtcagttg |
| 781 | ctcaatgtac ctataaccag accgttcagc tggatattac ggccttttta aagaccgtaa |
| 841 | agaaaaataa gcacaagttt tatccggcct ttattcacat tcttgcccgc ctgatgaatg |
| 901 | ctcatccgga gttccgtatg gcaatgaaag acggtgagct ggtgatatgg gatagtgttc |
| 961 | acccttgtta caccgttttc catgagcaaa ctgaaacgtt ttcatcgctc tggagtgaat |
| 1021 | accacgacga tttccggcag tttctacaca tatattcgca agatgtggcg tgttacggtg |
| 1081 | aaaacctggc ctatttccct aaagggttta ttgagaatat gtttttcgtc tcagccaatc |
| 1141 | cctgggtgag tttcaccagt tttgatttaa acgtggccaa tatggacaac ttcttcgccc |
| 1201 | ccgttttcac tatgggcaaa tattatacgc aaggcgacaa ggtgctgatg ccgctggcga |
| 1261 | ttcaggttca tcatgccgtt tgtgatggct tccatgtcgg cagaatgctt aatgaattac |
| 1321 | aacagtactg cgatgagtgg cagggcgggg cgtaacactc acgacgtgag tttggtttgt |
| 1381 | aagctgttat atgtaaaact agaacagctt atataactgg aaccataacc ttgccaccct |
| 1441 | ttattggaag cataagcttg ccgttgcggc aaagttatgg gtaaagtcac atctaagctg |
| 1501 | tctctagcct ttatttactg aatgggcagt accagactcg aactgatgac aatcccaatg |
| 1561 | gcgcgccgag cttggcgtaa tcatggtcat agctgtttcc tgtgtgaaat tgttatccgc |
| 1621 | tcacaattcc acacaacata cgagccggaa gcataaagtg taaagcctgg ggtgcctaat |
| 1681 | gagtgagcta actcacatta attgcgttgc gctcactgcc cgctttccag tcgggaaacc |
| 1741 | tgtcgtgcca gctgcattaa tgaatcggcc aacgcgcggg gagaggcggt ttgcgtattg |
| 1801 | ggcgctcttc cgcttcctcg ctcactgact cgctgcgctc ggtcgttcgg ctgcggcgag |
| 1861 | cggtatcagc tcactcaaag gcggtaatac ggttatccac agaatcaggg gataacgcag |
| 1921 | gaaagaacat gtgagcaaaa ggccagcaaa aggccaggaa ccgtaaaaag gccgcgttgc |
| 1981 | tggcgttttt ccataggctc cgcccccctg acgagcatca caaaatcaca aaaatcgacg |
| 2041 | ctcaagtcag aggtggcgaa acccgacagg actataaaga taccaggcgt ttccccctgg |
| 2101 | aagctccctc gtgcgctctc ctgttccgac cctgccgctt accggatacc tgtccgcctt |
| 2161 | tctcccttcg ggaagcgtgg cgctttctca tagctcacgc tgtaggtatc tcagttcggt |
| 2221 | gtaggtcgtt cgctccaagc tgggctgtgt gcacgaaccc cccgttcagc ccgaccgctg |
| 2281 | cgccttatcc ggtaactatc gtcttgagtc caacccggta agacacgact tatcgccact |
| 2341 | ggcagcagcc actggtaaca ggattagcag agcgaggtat gtaggcggtg ctacagagtt |
| 2401 | cttgaagtgg tggcctaact acggctacac tagaagaaca gtatttggta tctgcgctct |
| 2461 | gctgaagcca gttaccttcg gaaaaagagt tggtagctct tgatccggca aacaaaccac |
| 2521 | cgctggtagc ggtggttttt ttgtttgcaa gcagcagatt acgcgcagaa aaaaaggatc |
| 2581 | tcaagaagat cctttgatct tttctacggg gtctgacgct cagtggaacg aaaactcacg |
| 2641 | ttaagggatt ttggtcatga gattatcaaa aaggatcttc acctagatcc ttttaaatta |
| 2701 | aaaatgaagt tttaaatcaa tctaaagtat atatgagtaa acttggtctg acagttacca |
| 2761 | atgcttaatc agtgaggcac ctatctcagc gatctgtcta tttcgttcat ccatagttgc |
| 2821 | ctgactcccc gtcgtgtaga taactacgat acgggagggc ttaccatctg gccccagtgc |
| 2881 | tgcaatgata ccgcgagacc cacgctcacc ggctccagat ttatcagcaa taaaccagcc |
| 2941 | agccggaagg gccgagcgca gaagtggtcc tgcaacttta tccgcctcca tccagtctat |
| 3001 | taattgttgc cgggaagcta gagtaagtag ttcgccagtt aatagtttgc gcaacgttgt |
| 3061 | tgccattgct acaggcatcg tggtgtcacg ctcgtcgttt ggtatggctt cattcagctc |
| 3121 | cggttcccaa cgatcaaggc gagttacatg atcccccatg ttgtgcaaaa aagcggttag |
| 3181 | ctccttcggt cctccgatcg ttgtcagaag taagttggcc gcagtgttat cactcatggt |
| 3241 | tatggcagca ctgcataatt ctcttactgt catgccatcc gtaagatgct tttctgtgac |
| 3301 | tggtgagtac tcaaccaagt cattctgaga atagtgtatg cggcgaccga gttgctcttg |
| 3361 | cccggcgtca atacgggata ataccgcgcc acatagcaga actttaaaag tgctcatcat |
| 3421 | tggaaaacgt tcttcggggc gaaaactctc aaggatctta ccgctgttga gatccagttc |
| 3481 | gatgtaaccc actcgtgcac ccaactgatc ttcagcatct tttactttca ccagcgtttc |
| 3541 | tgggtgagca aaaacaggaa ggcaaaatgc cgcaaaaaag ggaataaggg cgacacggaa |
| 3601 | atgttgaata ctcatactct tcctttttca atattattga agcatttatc agggttattg |
| 3661 | tctcatgagc ggatacatat ttgaatgtat ttagaaaaat aaacaaatag gggttccgcg |
| 3721 | cacatttccc cgaaaagtgc cacctgacgt ctaagaaacc attattatca tgacattaac |
| 3781 | ctataaaaat aggcgtatca cgaggccctt tcgtc // |

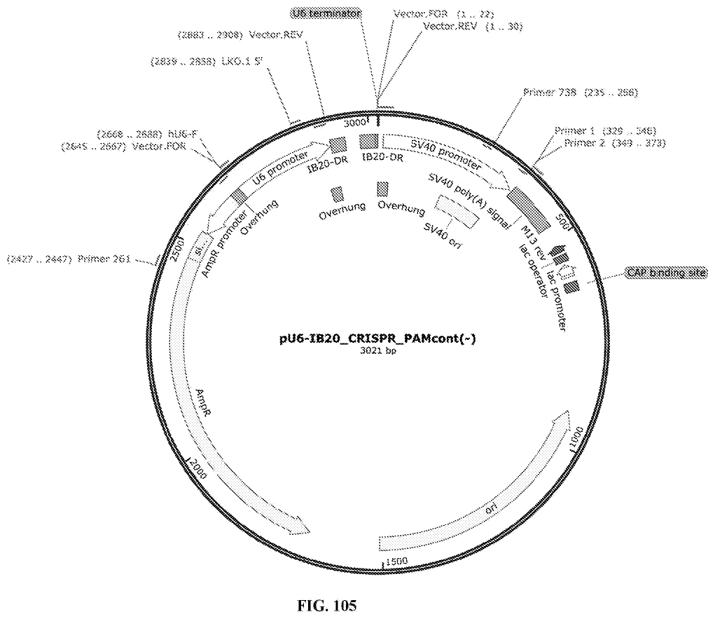

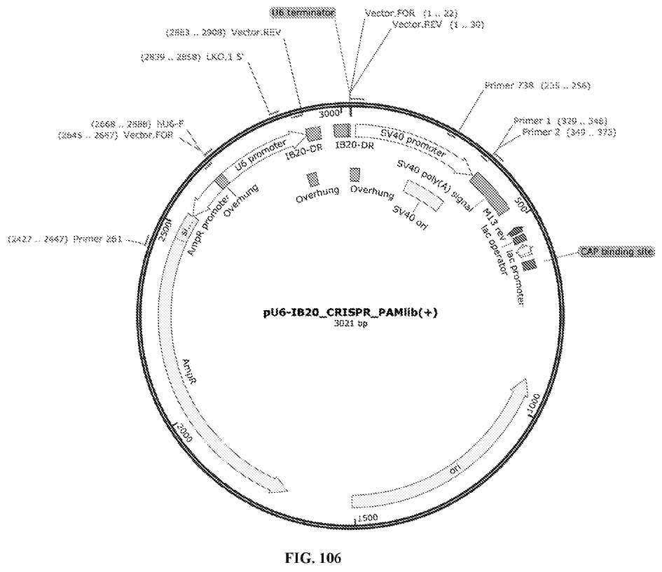

Plasmids for characterizing PAM (pU6-IB20_CRISPR_PAMcont(−) and pU6-IB20_CRISPR PAMlib(+)) are shown in FIGS. 105 and 106. Sequences and features are below.

| pU6-IB20_CRISPR_PAMcont(−) | |
|---|---|
| misc_feature | 1 . . . 30 |
| | /label = Overhung |
| primer_bind | 1 . . . 30 |
| | /label = Vector.REV |
| primer_bind | 1 . . . 22 |
| | /label = Vector.FOR |
| promoter | 14 . . . 343 |
| | /label = SV40 promoter |
| | /note = “SV40 enhancer and early promoter” |
| rep_origin | 194 . . . 329 |
| | /label = SV40 ori |
| | /note = “SV40 origin of replication” |
| primer_bind | complement(235 . . . 256) |
| | /label = Primer 738 |
| primer_bind | complement(329 . . . 348) |
| | /label = Primer 1 |
| polyA_signal | 349 . . . 470 |
| | /label = SV40 poly(A) signal |
| | /note = “SV40 polyadenylation signal” |
| primer_bind | 349 . . . 373 |
| | /label = Primer 2 |
| primer_bind | complement(519 . . . 535) |
| | /label = M13 rev |
| | /note = “common sequencing primer, one of multiple similar |
| | variants” |
| protein_bind | 543 . . . 559 |
| | /label = lac operator |
| | /bound_moiety = “lac repressor encoded by lacI” |
| | /note = “The lac repressor binds to the lac operator to |
| | inhibit transcription in E. coli. This inhibition can be |
| | relieved by adding lactose or |
| | isopropyl-beta-D-thiogalactopyranoside (IPTG).” |
| promoter | complement(567 . . . 597) |
| | /label = lac promoter |
| | /note = “promoter for the E. coli lac operon” |
| protein_bind | 612 . . . 633 |
| | /label = CAP binding site |
| | /bound_moiety = “E. coli catabolite activator protein” |
| | /note = “CAP binding activates transcription in the presence |
| | of cAMP.” |
| rep_origin | complement(921 . . . 1506) |
| | /direction = LEFT |
| | /label = ori |
| | /note = “high-copy-number ColE1/pMB1/pBR322/pUC origin of |
| | replication” |
| CDS | complement(1677 . . . 2537) |
| | /codon_start = 1 |
| | /gene = “bla” |
| | /product = “beta-lactamase” |
| primer_bind | 2427 . . . 2447 |
| | /label = Primer 261 |
| promoter | complement(2538 . . . 2642) |
| | /gene = “bla” |
| | /label = AmpR promoter |
| misc_feature | 2643 . . . 2667 |
| | /label = Overhung |
| primer_bind | complement(2645 . . . 2667) |
| | /label = Vector.FOR |
| promoter | 2668 . . . 2908 |
| | /label = U6 promoter |
| | /note = “RNA polymerase III promoter for human U6 snRNA” |
| | primer_bind 2668 . . . 2688 |
| | /label = hU6-F |
| | /note = “Human U6 promoter, forward primer” |
| primer_bind | 2839 . . . 2858 |
| | /label = LKO.1 5′ |
| | /note = “Human U6 promoter, forward primer” |
| misc_feature | 2879 . . . 2908 |
| | /label = Overhung |
| primer_bind | complement(2883. .2908) |
| | /label = Vector.REV |
| misc_feature | 2909 . . . 2944 |
| | /label = IB20-DR |
| misc_feature | 2980 . . . 3015 |
| | /label = IB20-DR |
| misc_feature | 3016 . . . 3021 |
| | /label = U6 terminator |
| (SEQ ID NO: 100) | |
| 1 | aattctgtgg aatgtgtgtc agttagggtg tggaaagtcc ccaggctccc cagcaggcag |
| 61 | aagtatgcaa agcatgcatc tcaattagtc agcaaccagg tgtggaaagt ccccaggctc |
| 121 | cccagcaggc agaagtatgc aaagcatgca tctcaattag tcagcaacca tagtcccgcc |
| 181 | cctaactccg cccatcccgc ccctaactcc gcccagttcc gcccattctc cgccccatgg |
| 241 | ctgactaatt ttttttattt atgcagaggc cgaggccgcc tctgcctctg agctattcca |
| 301 | gaagtagtga ggaggctttt ttggaggcct aggcttttgc aaaaagctaa cttgtttatt |
| 361 | gcagcttata atggttacaa ataaagcaat agcatcacaa atttcacaaa taaagcattt |
| 421 | ttttcactgc attctagttg tggtttgtcc aaactcatca atgtatctta tcatgtctgt |
| 481 | ataccgtcga cctctagcta gagcttggcg taatcatggt catagctgtt tcctgtgtga |
| 541 | aattgttatc cgctcacaat tccacacaac atacgagccg gaagcataaa gtgtaaagcc |
| 601 | tggggtgcct aatgagtgag ctaactcaca ttaattgcgt tgcgctcact gcccgctttc |
| 661 | cagtcgggaa acctgtcgtg ccagctgcat taatgaatcg gccaacgcgc ggggagaggc |
| 721 | ggtttgcgta ttgggcgctc ttccgcttcc tcgctcactg actcgctgcg ctcggtcgtt |
| 781 | cggctgcggc gagcggtatc agctcactca aaggcggtaa tacggttatc cacagaatca |
| 841 | ggggataacg caggaaagaa catgtgagca aaaggccagc aaaaggccag gaaccgtaaa |
| 901 | aaggccgcgt tgctggcgtt tttccatagg ctccgccccc ctgacgagca tcacaaaaat |
| 961 | cgacgctcaa gtcagaggtg gcgaaacccg acaggactat aaagatacca ggcgtttccc |
| 1021 | cctggaagct ccctcgtgcg ctctcctgtt ccgaccctgc cgcttaccgg atacctgtcc |
| 1081 | gcctttctcc cttcgggaag cgtggcgctt tctcatagct cacgctgtag gtatctcagt |
| 1141 | tcggtgtagg tcgttcgctc caagctgggc tgtgtgcacg aaccccccgt tcagcccgac |
| 1201 | cgctgcgcct tatccggtaa ctatcgtctt gagtccaacc cggtaagaca cgacttatcg |
| 1261 | ccactggcag cagccactgg taacaggatt agcagagcga ggtatgtagg cggtgctaca |
| 1321 | gagttcttga agtggtggcc taactacggc tacactagaa gaacagtatt tggtatctgc |
| 1381 | gctctgctga agccagttac cttcggaaaa agagttggta gctcttgatc cggcaaacaa |
| 1441 | accaccgctg gtagcggttt ttttgtttgc aagcagcaga ttacgcgcag aaaaaaagga |
| 1501 | tctcaagaag atcctttgat cttttctacg gggtctgacg ctcagtggaa cgaaaactca |
| 1561 | cgttaaggga ttttggtcat gagattatca aaaaggatct tcacctagat ccttttaaat |
| 1621 | taaaaatgaa gttttaaatc aatctaaagt atatatgagt aaacttggtc tgacagttac |
| 1681 | caatgcttaa tcagtgaggc acctatctca gcgatctgtc tatttcgttc atccatagtt |
| 1741 | gcctgactcc ccgtcgtgta gataactacg atacgggagg gcttaccatc tggccccagt |
| 1801 | gctgcaatga taccgcgaga cccacgctca ccggctccag atttatcagc aataaaccag |
| 1861 | ccagccggaa gggccgagcg cagaagtggt cctgcaactt tatccgcctc catccagtct |
| 1921 | attaattgtt gccgggaagc tagagtaagt agttcgccag ttaatagttt gcgcaacgtt |
| 1981 | gttgccattg ctacaggcat cgtggtgtca cgctcgtcgt ttggtatggc ttcattcagc |
| 2041 | tccggttccc aacgatcaag gcgagttaca tgatccccca tgttgtgcaa aaaagcggtt |
| 2101 | agctccttcg gtcctccgat cgttgtcaga agtaagttgg ccgcagtgtt atcactcatg |
| 2161 | gttatggcag cactgcataa ttctcttact gtcatgccat ccgtaagatg cttttctgtg |
| 2221 | actggtgagt actcaaccaa gtcattctga gaatagtgta tgcggcgacc gagttgctct |
| 2281 | tgcccggcgt caatacggga taataccgcg ccacatagca gaactttaaa agtgctcatc |
| 2341 | attggaaaac gttcttcggg gcgaaaactc tcaaggatct taccgctgtt gagatccagt |
| 2401 | tcgatgtaac ccactcgtgc acccaactga tcttcagcat cttttacttt caccagcgtt |
| 2461 | tctgggtgag caaaaacagg aaggcaaaat gccgcaaaaa agggaataag ggcgacacgg |
| 2521 | aaatgttgaa tactcatact cttccttttt caatattatt gaagcattta tcagggttat |
| 2581 | tgtctcatga gcggatacat atttgaatgt atttagaaaa ataaacaaat aggggttccg |
| 2641 | cgcacatttc cccgaaaagt gccacctgag ggcctatttc ccatgattcc ttcatatttg |
| 2701 | catatacgat acaaggctgt tagagagata attagaatta atttgactgt aaacacaaag |
| 2761 | atattagtac aaaatacgtg acgtagaaag taataatttc ttgggtagtt tgcagtttta |
| 2821 | aaattatgtt ttaaaatgga ctatcatatg cttaccgtaa cttgaaagta tttcgatttc |
| 2881 | ttggctttat atatcttgtg gaaaggacgt gagtcgtggc tttatgccgt taggcgttgc |
| 3001 | aaccatc tatgtatagc tacgatggaa tagtaaaggg tgagtcgtgg ctttatgccg |
| 3001 | ttaggcgttg ctcaattttt t // |
| pU6-IB20_CRISPR_PAMlib(+) | |
| misc_feature | 1 . . . 30 |
| | /label = Overhung |
| primer_bind | 1 . . . 30 |
| | /label = Vector.REV |
| primer_bind | 1 . . . 22 |
| | /label = Vector.FOR |
| promoter | 14 . . . 343 |
| | /label = SV40 promoter |
| | /note = “SV40 enhancer and early promoter” |
| rep_origin | 194 . . . 329 |
| | /label = SV40 ori |
| | /note = “SV40 origin of replication” |
| primer_bind | complement(235 . . . 256) |
| | /label = Primer 738 |
| primer_bind | complement(329 . . . 348) |
| | /label = Primer 1 |
| polyA_signal | 349 . . . 470 |
| | /label = SV40 poly(A) signal |
| | /note = “SV40 polyadenylation signal” |
| primer_bind | 349 . . . 373 |
| | /label = Primer 2 |
| primer_bind | complement(519 . . . 535) |
| | /label = M13 rev |
| | /note = “common sequencing primer, one of multiple similar |
| | variants” |
| protein_bind | 543 . . . 559 |
| | /label = lac operator |
| | /bound_moiety = “lac repressor encoded by lacI” |
| | /note = “The lac repressor binds to the lac operator to |
| | inhibit transcription in E. coli. This inhibition can be |
| | relieved by adding lactose or |
| | isopropyl-beta-D-thiogalactopyranoside (IPTG).” |
| promoter | complement(567 . . . 597) |
| | /label = lac promoter |
| | /note = “promoter for the E. coli lac operon” |
| protein_bind | 612 . . . 633 |
| | /label = CAP binding site |
| | /bound_moiety = “E. coli catabolite activator protein” |
| | /note = “CAP binding activates transcription in the presence |
| | ofc AMP.” |
| rep_origin | complement(921 . . . 1506) |
| | /direction = LEFT |
| | /label = ori |
| | /note = “high-copy-number ColE1/pMB1/pBR322/pUC origin of |
| | replication” |
| CDS | complement(1677 . . . 2537) |
| | /codon_start = 1 |
| | /gene = “bla” |
| | /product = “beta-lactamase” |
| | /label = AmpR |
| | /note = “confers resistance to ampicillin, carbenicillin, and |
| | related antibiotics” |
| | /translation =” |
| primer_bind | 2427 . . . 2447 |
| | /label = Primer 261 |
| promoter | complement(2538 . . . 2642) |
| | /gene = “bla” |
| | /label = AmpR promoter |
| misc_feature | 2643 . . . 2667 |
| | /label = Overhung |
| primer_bind | complement(2645. .2667) |
| | /label = Vector.FOR |
| promoter | 2668 . . . 2908 |
| | /label = U6 promoter |
| | /note = “RNA polymerase III promoter for human U6 snRNA” |
| primer_bind | 2668 . . . 2688 |
| | /label = hU6-F |
| | /note = “Human U6 promoter, forward primer” |
| primer_bind | 2839 . . . 2858 |
| | /label = LKO.1 5′ |
| | /note = “Human U6 promoter, forward primer” |
| misc_feature | 2879 . . . 2908 |
| | /label = Overhung |
| primer_bind | complement(2883. .2908) |
| | /label = Vector.REV |
| misc_feature | 2909 . . . 2944 |
| | /label = IB20-DR |
| misc_feature | 2980 . . . 3015 |
| | /label = IB20-DR |
| misc_feature | 3016 . . . 3021 |
| | /label = U6 terminator |
| ORIGIN | |
| (SEQ ID NO: 101) | |
| 1 | aattctgtgg aatgtgtgtc agttagggtg tggaaagtcc ccaggctccc cagcaggcag |
| 61 | aagtatgcaa agcatgcatc tcaattagtc agcaaccagg tgtggaaagt ccccaggctc |
| 121 | cccagcaggc agaagtatgc aaagcatgca tctcaattag tcagcaacca tagtcccgcc |
| 181 | cctaactccg cccatcccgc ccctaactcc gcccagttcc gcccattctc cgccccatgg |
| 241 | ctgactaatt ttttttattt atgcagaggc cgaggccgcc tctgcctctg agctattcca |
| 301 | gaagtagtga ggaggctttt ttggaggcct aggcttttgc aaaaagctaa cttgtttatt |
| 361 | gcagcttata atggttacaa ataaagcaat agcatcacaa atttcacaaa taaagcattt |
| 421 | ttttcactgc attctagttg tggtttgtcc aaactcatca atgtatctta tcatgtctgt |
| 481 | ataccgtcga cctctagcta gagcttggcg taatcatggt catagctgtt tcctgtgtga |
| 541 | aattgttatc cgctcacaat tccacacaac atacgagccg gaagcataaa gtgtaaagcc |
| 601 | tggggtgcct aatgagtgag ctaactcaca ttaattgcgt tgcgctcact gcccgctttc |
| 661 | cagtcgggaa acctgtcgtg ccagctgcat taatgaatcg gccaacgcgc ggggagaggc |
| 721 | ggtttgcgta ttgggcgctc ttccgcttcc tcgctcactg actcgctgcg ctcggtcgtt |
| 781 | cggctgcggc gagcggtatc agctcactca aaggcggtaa tacggttatc cacagaatca |
| 841 | ggggataacg caggaaagaa catgtgagca aaaggccagc aaaaggccag gaaccgtaaa |
| 901 | aaggccgcgt tgctggcgtt tttccatagg ctccgccccc ctgacgagca tcacaaaaat |
| 961 | cgacgctcaa gtcagaggtg gcgaaacccg acaggactat aaagatacca ggcgtttccc |
| 1021 | cctggaagct ccctcgtgcg ctctcctgtt ccgaccctgc cgcttaccgg atacctgtcc |
| 1081 | gcctttctcc cttcgggaag cgtggcgctt tctcatagct cacgctgtag gtatctcagt |
| 1141 | tcggtgtagg tcgttcgctc caagctgggc tgtgtgcacg aaccccccgt tcagcccgac |
| 1201 | cgctgcgcct tatccggtaa ctatcgtctt gagtccaacc cggtaagaca cgacttatcg |
| 1261 | ccactggcag cagccactgg taacaggatt agcagagcga ggtatgtagg cggtgctaca |
| 1321 | gagttcttga agtggtggcc taactacggc tacactagaa gaacagtatt tggtatctgc |
| 1381 | gctctgctga agccagttac cttcggaaaa agagttggta gctcttgatc cggcaaacaa |
| 1441 | accaccgctg gtagcggttt ttttgtttgc aagcagcaga ttacgcgcag aaaaaaagga |
| 1501 | tctcaagaag atcctttgat cttttctacg gggtctgacg ctcagtggaa cgaaaactca |
| 1561 | cgttaaggga ttttggtcat gagattatca aaaaggatct tcacctagat ccttttaaat |
| 1621 | taaaaatgaa gttttaaatc aatctaaagt atatatgagt aaacttggtc tgacagttac |
| 1681 | caatgcttaa tcagtgaggc acctatctca gcgatctgtc tatttcgttc atccatagtt |
| 1741 | gcctgactcc ccgtcgtgta gataactacg atacgggagg gcttaccatc tggccccagt |
| 1801 | gctgcaatga taccgcgaga cccacgctca ccggctccag atttatcagc aataaaccag |
| 1861 | ccagccggaa gggccgagcg cagaagtggt cctgcaactt tatccgcctc catccagtct |
| 1921 | attaattgtt gccgggaagc tagagtaagt agttcgccag ttaatagttt gcgcaacgtt |
| 1981 | gttgccattg ctacaggcat cgtggtgtca cgctcgtcgt ttggtatggc ttcattcagc |
| 2041 | tccggttccc aacgatcaag gcgagttaca tgatccccca tgttgtgcaa aaaagcggtt |
| 2101 | agctccttcg gtcctccgat cgttgtcaga agtaagttgg ccgcagtgtt atcactcatg |
| 2161 | gttatggcag cactgcataa ttctcttact gtcatgccat ccgtaagatg cttttctgtg |
| 2221 | actggtgagt actcaaccaa gtcattctga gaatagtgta tgcggcgacc gagttgctct |
| 2281 | tgcccggcgt caatacggga taataccgcg ccacatagca gaactttaaa agtgctcatc |
| 2341 | attggaaaac gttcttcggg gcgaaaactc tcaaggatct taccgctgtt gagatccagt |
| 2401 | tcgatgtaac ccactcgtgc acccaactga tcttcagcat cttttacttt caccagcgtt |
| 2461 | tctgggtgag caaaaacagg aaggcaaaat gccgcaaaaa agggaataag ggcgacacgg |
| 2521 | aaatgttgaa tactcatact cttccttttt caatattatt gaagcattta tcagggttat |
| 2581 | tgtctcatga gcggatacat atttgaatgt atttagaaaa ataaacaaat aggggttccg |
| 2641 | cgcacatttc cccgaaaagt gccacctgag ggcctatttc ccatgattcc ttcatatttg |
| 2701 | catatacgat acaaggctgt tagagagata attagaatta atttgactgt aaacacaaag |
| 2761 | atattagtac aaaatacgtg acgtagaaag taataatttc ttgggtagtt tgcagtttta |
| 2821 | aaattatgtt ttaaaatgga ctatcatatg cttaccgtaa cttgaaagta tttcgatttc |
| 2881 | ttggctttat atatcttgtg gaaaggacgt gagtcgtggc tttatgccgt taggcgttgc |
| 3001 | agagaag tcatttaata aggccactgt taaacgtagg tgagtcgtgg ctttatgccg |
| 3001 | ttaggcgttg ctcaattttt t // |

### Example 20

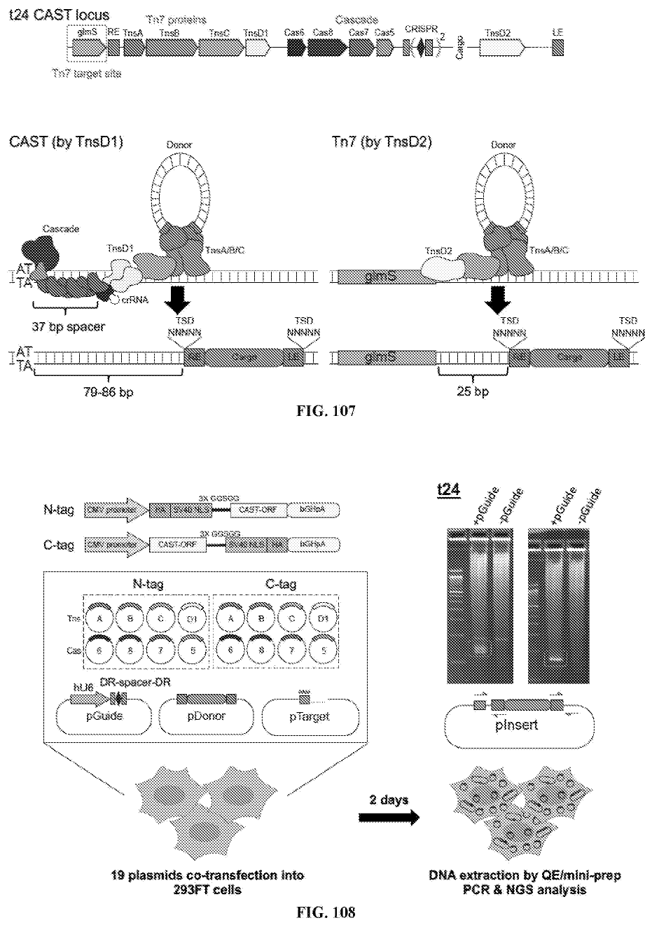

Example CAST 1-b T24 characterization in bacteria is summarized in FIG. 107. In this example, when the system comprises TnsD2, the donor polynucleotide may be inserted by the Tn7 transposases without Cas proteins. In such cases, the target sequence on the locus of the Tn7 transposase, e.g., next to glmS gene. When the system comprises TnsD1, the donor polynucleotide may be inserted by CAST, i.e., Tn7 transposases guided by the Cas protein and guide molecule. In such cases, the target sequence can be any sequence determined by the specificity of the Cas protein and the guide molecule. The Cas protein and the guide molecule may be designed to insert the donor to a desired target sequence.

Applicants tested T24 CAST function in targeting plasmids in mammalian cells. Plasmids expressing the transposases, Cas proteins, guide molecules, as well as plasmids providing donor polynucleotide and target sequences were co-transfected to 293FT cells. After 2 days, DNA was extracted from the cells by QE/mini-prep and insertion of donor polynucleotide in the target sequence was determined by PCR and next generation sequencing. The plasmids design and results were shown in FIG. 108.

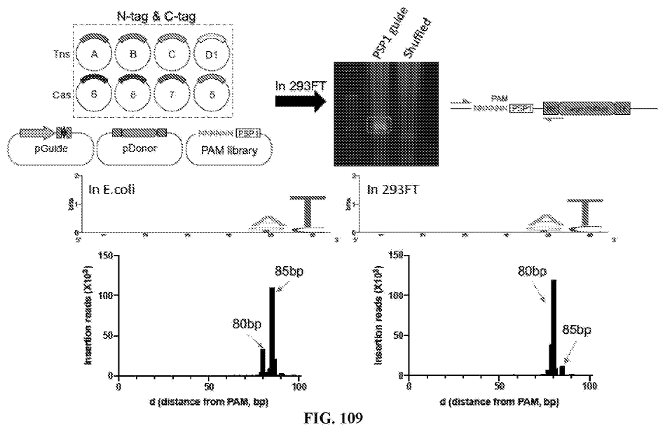

PAM was screened for T24 CAST. Plasmids expressing transposases and guide molecules and plasmids providing donor polynucleotides and target sequences with PAM libraries were co-transfected in 293FT cells. PAM was screened and result is shown in FIG. 109.

New functional CAST orthologs (IB29 and IB18) were identified by PAM screening (FIG. 110). IB29 is the same as IB28 described herein.

Two subtypes of Type 1-b CAST were identified. The system of one of the subtypes (e.g., IB20) comprises a transposases that has activities of both TnsA and TnsB in a typical Tn7 system (e.g., shown as tnsAB in IB20) (FIG. 111). FIG. 112 shows example type I-b CAST loci. Four of these systems (T24, IB29, IB18, and IB20) were found to be able to target plasmids in mammalian cells.

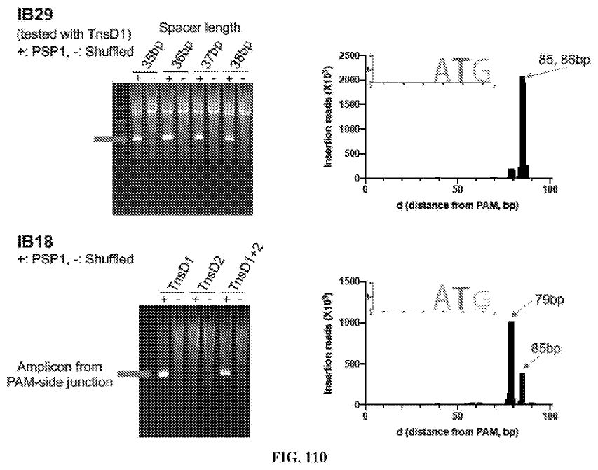

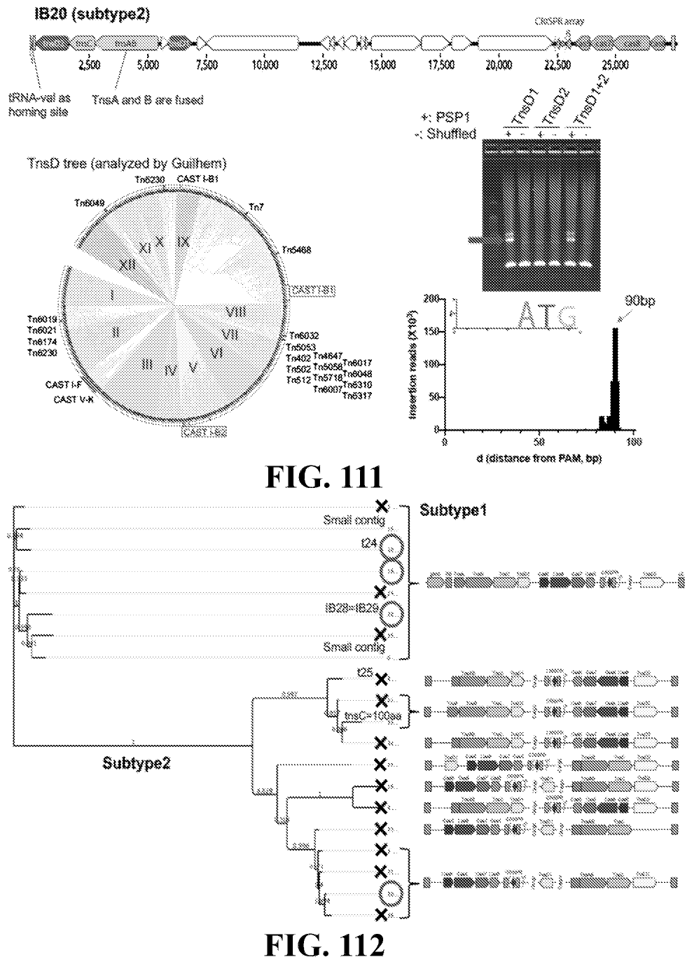

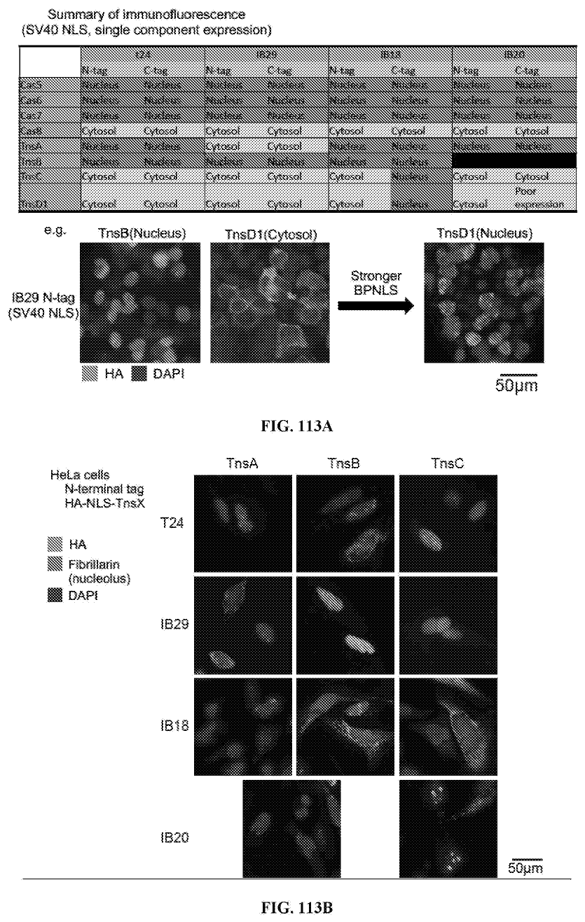

NLSs were fused to the components of example Type I-b CAST systems and the subcellular localization of the components were tested by immunofluorescence (FIGS. 113A-113B).

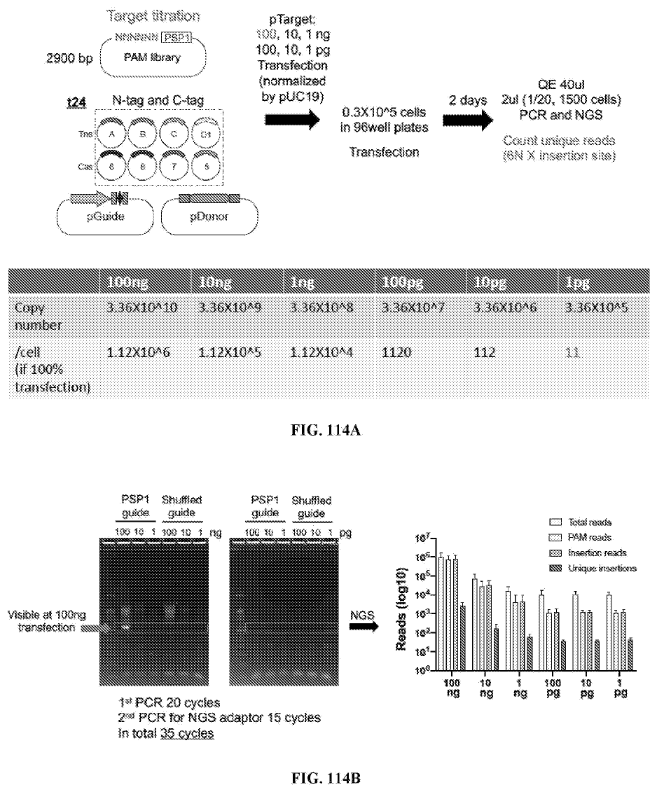

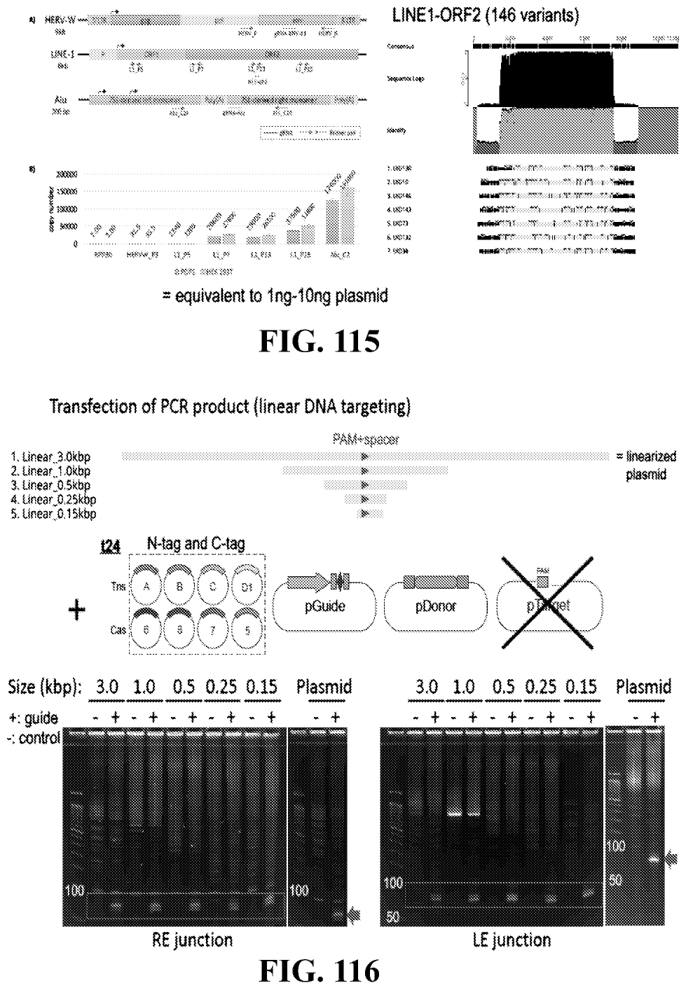

Copy numbers of the plasmids needed for the T24 system to be active were tested (FIG. 114A-114B). Applicants also tested targeting LINE1-ORF2 by each of the four example CAST systems with 24 guide molecules (FIG. 115).

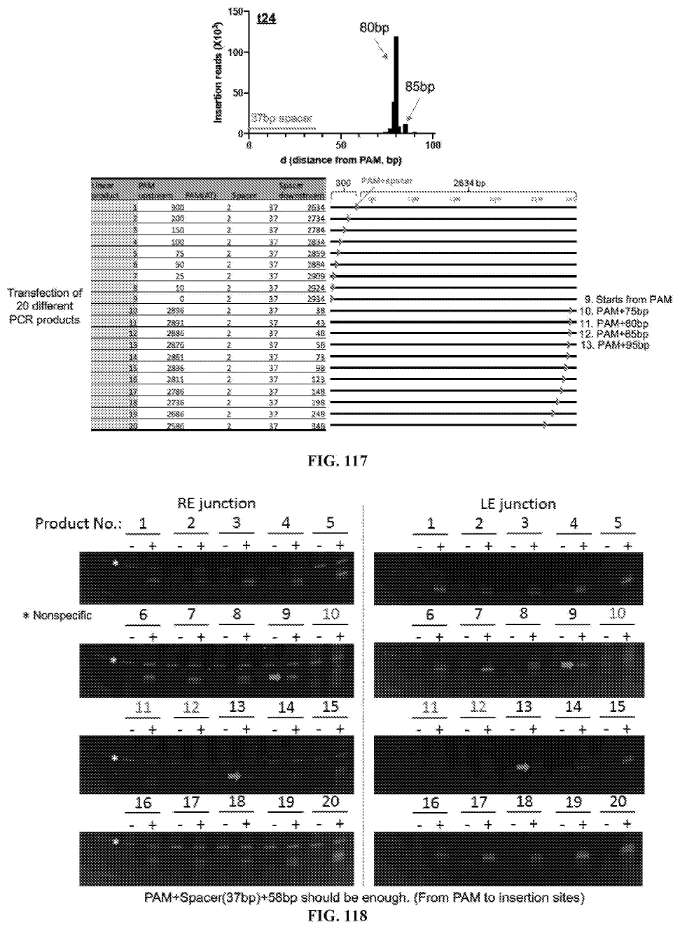

Applicants tested the activity of the example CAST-1b systems in targeting supercoiled (plasmids) and linear DNA. The CAST systems were active in targeting both super coiled and linear DNA targets (FIG. 116). Applicants further tested the effect of target site positions on the activity of the CAST-1b systems. Different sizes of the target sequences were tested, including linear targets comprising up to 300 bp upstream (5′ of PAM) and linear targets comprising up to 348 bp downstream (3′ of PAM) (FIG. 117). The minimal fragment size for Type I-b CAST function in 293FT cells (FIG. 118).

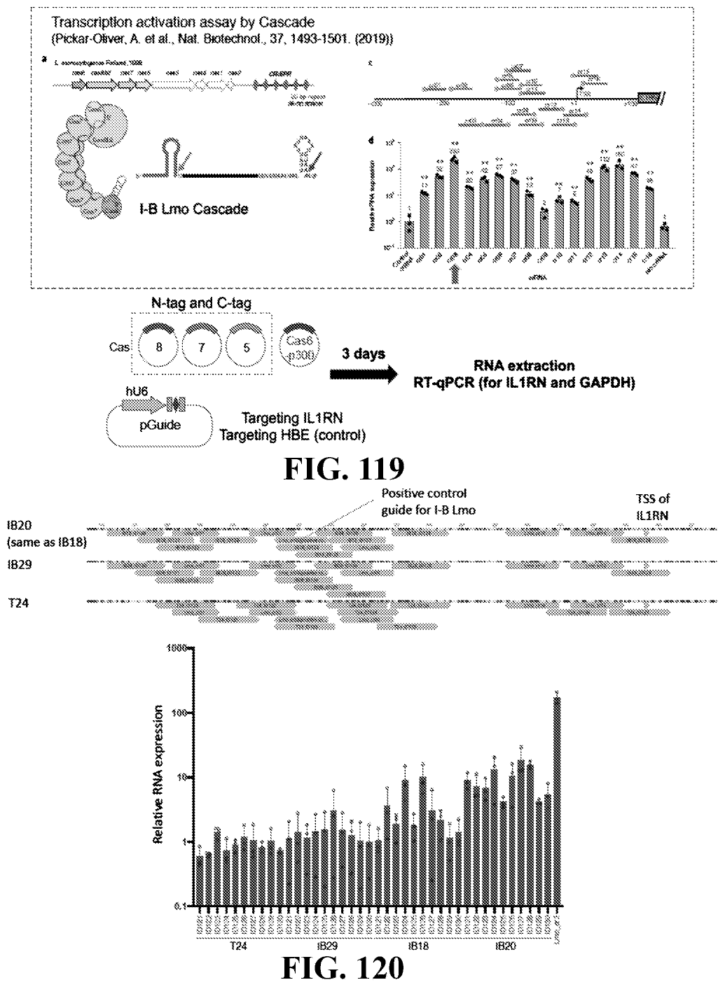

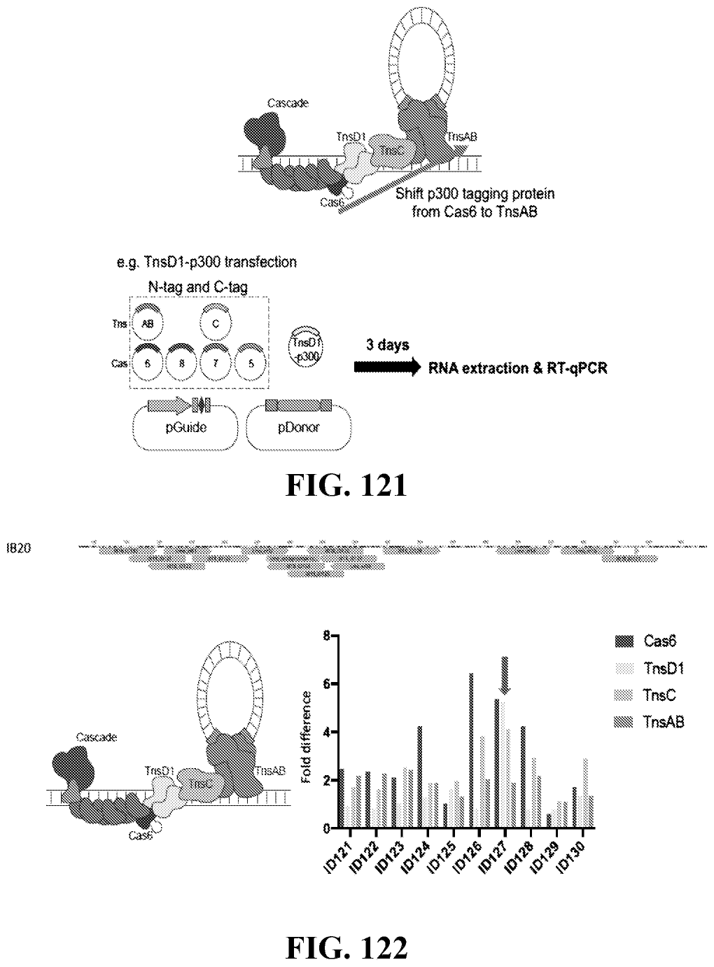

Nucleosome accessibility of the Cas proteins in the Type I-b CAST systems was tested by transcription activation assay as described in Pickar-Oliver et al. Nat. Biotechnol. 37, 1493-1501 (2019) (FIGS. 119-120). In another test, the individual Cas proteins in IB20 was fused with p300 and the transcription activation was tested (FIGS. 121-122).

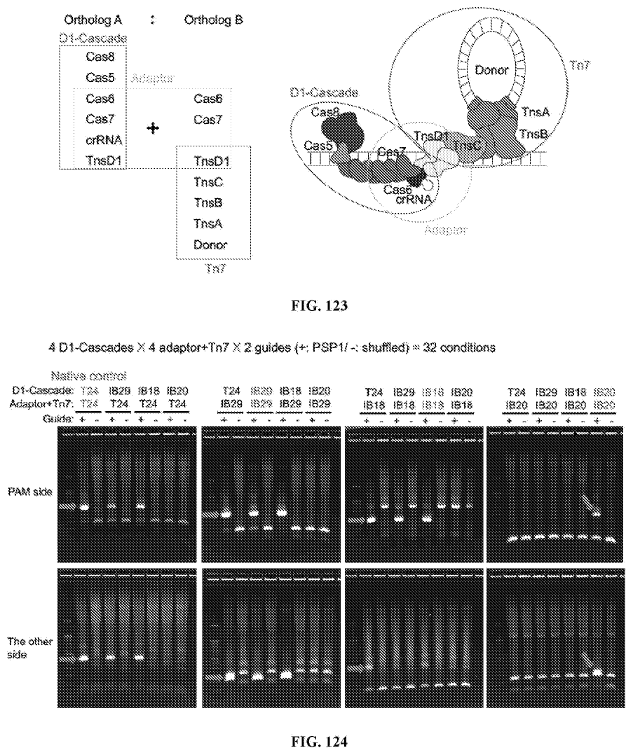

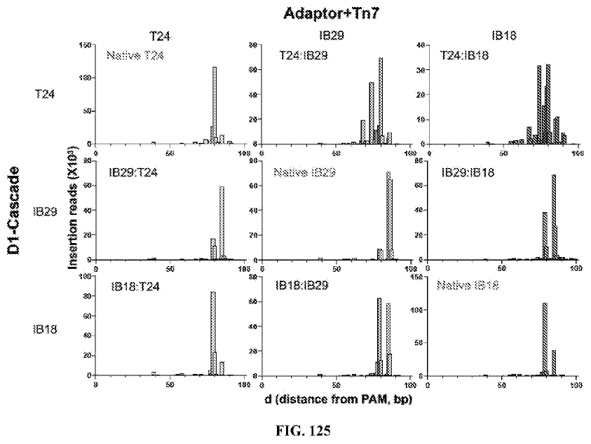

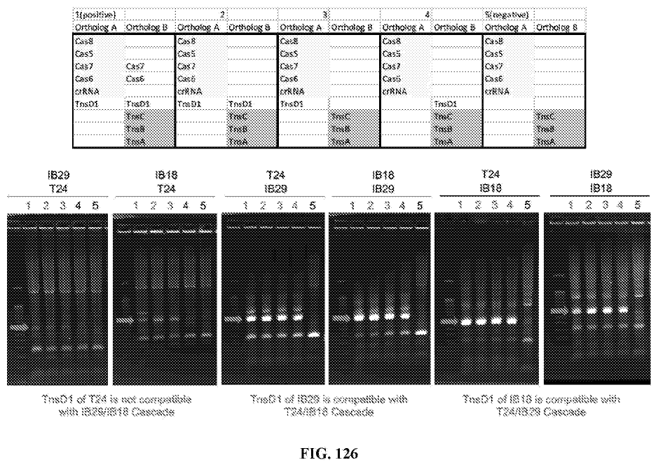

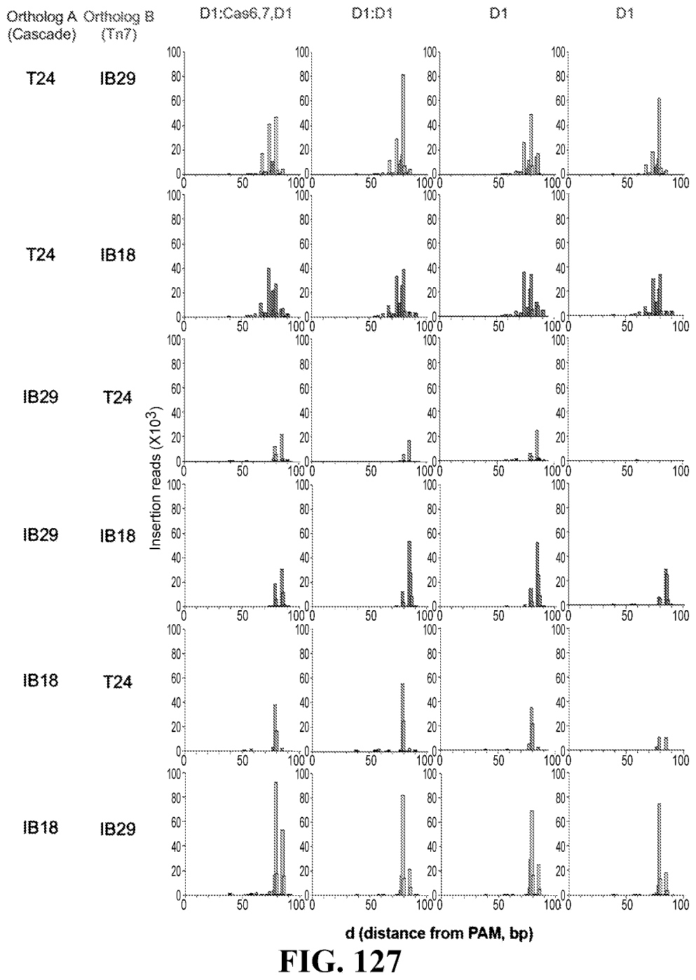

Chimeric Type I-b CAST systems were tested. In this examples, the chimeric systems comprise some components derived from a first species and other components derived from a second species different from the first species. FIG. 123 shows an example chimeric CAST system. The activities of the chimeric CAST systems in plasmid targeting in mammalian cells were tested (FIG. 124), and the insertion positions were determined (FIG. 125). In some of the examples, including the TnsD1 from both orthologs had an impact of the activity of the chimeric CAST (FIGS. 126 and 127).

### Example 25

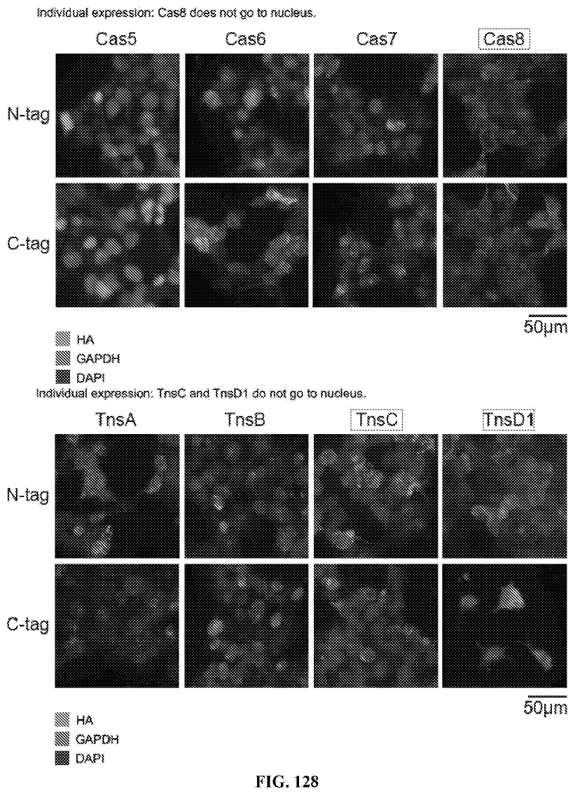

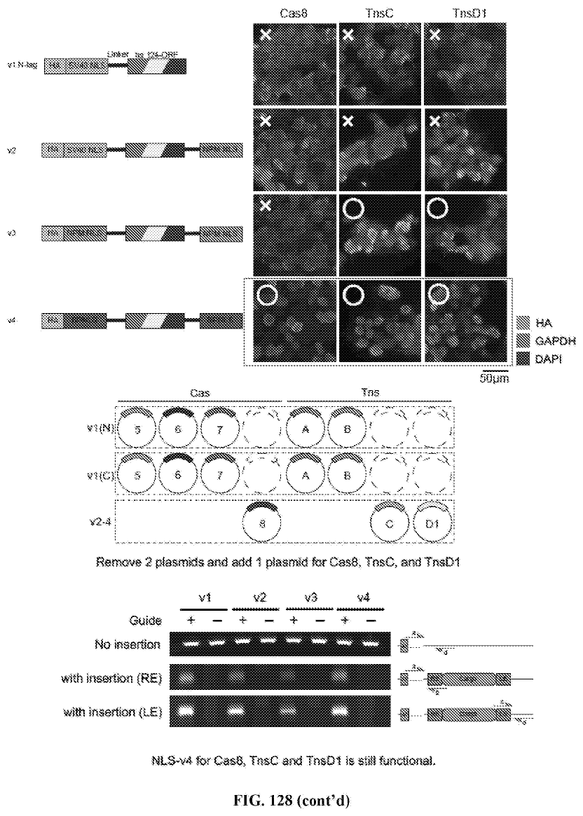

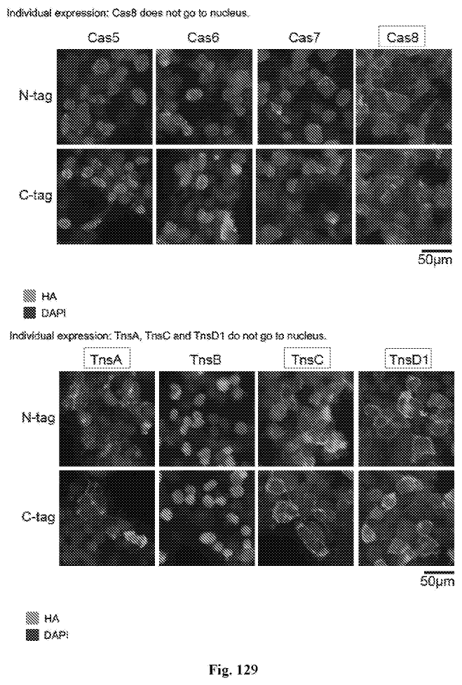

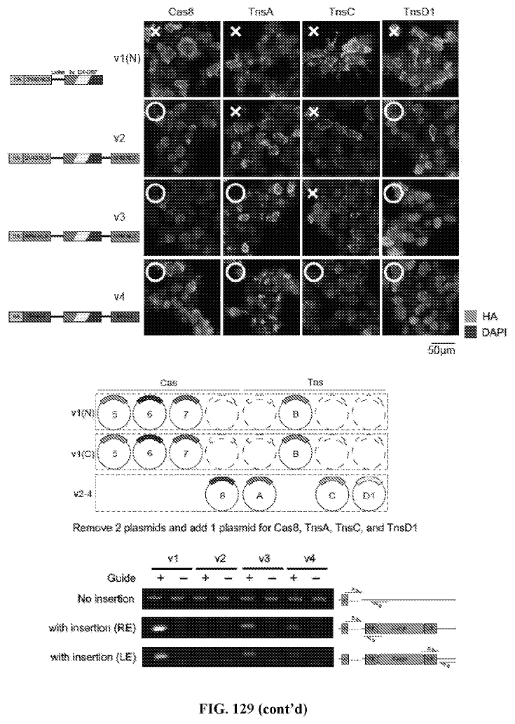

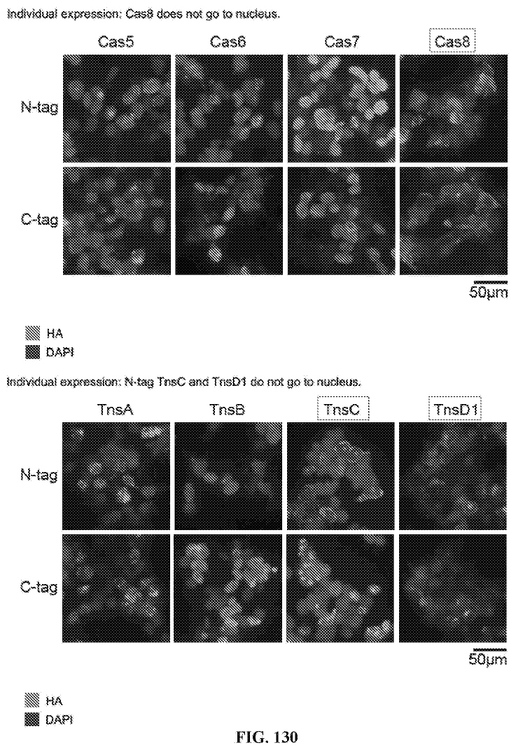

The activities of the CAST systems with new NLSs in plasmid targeting were tested (FIG. 128 for T24; FIG. 129 for IB29; FIG. 130 for IB18).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered system, the system comprising:
a. one or more CRISPR-associated Tn7 transposases;
b. one or more Type I-B Cas proteins; and
c. a guide molecule capable of complexing with the Type I-B Cas protein and directing binding of the guide-Cas protein complex to a target polynucleotide.

2. The system of claim 1, wherein the one or more CRISPR-associated Tn7 transposases comprise TnsA, TnsB, TnsC, and/or TniQ.

3. The system of claim 2, wherein the Tn7 transposases comprise TnsA, TnsB, TnsC, a first TniQ and a second TniQ, wherein the first and second TniQ are different.

4. The system of claim 2, wherein the Tn7 transposases comprise comprises TnsA, TnsB, TnsC, and a TniQ.

5. The system of claim 4, wherein the TniQ comprises a DNA-binding domain.

6. The system of claim 5, wherein the DNA-binding domain is at C-terminus of the TniQ.

7. The system of claim 1, wherein the one or more CRISPR-associated Tn7 transposases comprise TnsA, TnsB, TnsC, and/or TnsD.

8. The system of claim 7, wherein the Tn7 transposases comprise TnsA, TnsB, TnsC, a first TnsD and a second TnsD, wherein the first and second TnsD are different.

9. The system of claim 7, wherein the Tn7 transposase comprises TnsA, TnsB, TnsC and a TnsD.

10. The system of claim 9, wherein the TnsD comprises a DNA-binding domain.

11. The system of claim 9, wherein the TnsA and TnsB are comprised in a single protein.

12. The system of claim 9, wherein the Tn7 transposase comprises:
a. TnsA, TnsB, TnsC and TnsD1;
b. TnsA, TnsB, TnsC and TnsD2; or
c. TnsA, TnsB, TnsC, TnsD, and TnsD2.

13. The system of claim 1, wherein the one or more Type I-B Cas proteins comprises Cas5, Cas6, Cas7, and/or Cas 8.

14. The system of claim 1, wherein the one or more Type I-B Cas proteins comprises Cas 8b.

15. The system of claim 14, wherein the Cas8b is Cas8b3.

16. The system of claim 1, wherein the one or more CRISPR-associated Tn7 transposases and/or the one or more Type I-B Cas proteins are from or originated from Anabaena variabilis.

17. The system of claim 1, wherein the one or more Type I-B Cas proteins lacks nuclease activity.

18. The system of claim 1, further comprising a donor polynucleotide.

19. The system of claim 18, wherein the donor polynucleotide comprises a polynucleotide insert, a left element sequence, and a right element sequence.

20. The system of claim 18, wherein the donor polynucleotide:
a. introduces one or more mutations to the target polynucleotide;
b. corrects a premature stop codon in the target polynucleotide;
c. disrupts a splicing site;
d. restores a splicing site; or
e. a combination thereof.

21. The system of claim 20, wherein the one or more mutations introduced by the donor polynucleotide comprises substitutions, deletions, insertions, or a combination thereof.

22. The system of claim 20, wherein the one or more mutations causes a shift in an open reading frame on the target polynucleotide.

23. The system of claim 18, wherein the donor polynucleotide is between 100 bases and 30 kb in length.

24. The system of claim 1, wherein the target polynucleotide comprises a protospacer adjacent motif (PAM).

25. The system of claim 24, wherein the PAM is AT or ATG.

26. The system of claim 25, wherein the donor polynucleotide is inserted to the target polynucleotide at a site no more than 80, no more 90, no more 100, no more 200, or no more 300 bp 3′ of the PAM.

27. The system of claim 25, wherein the donor polynucleotide is inserted to the target polynucleotide at a site no more than 80, no more 90, no more 100, no more 200, or no more 300 bp 5′ of the PAM.

28. The system of claim 1, wherein the target polynucleotide is linear, circular, or genomic DNA.

29. The system of claim 1, wherein the one or more Tn7 transposases is derived from a first species and the one or more Type I-B Cas proteins is derived from a second species different from the first species.

30. The system of claim 29, comprising a first TnsD1 derived from the first species and a second TnsD2 derived from the second species.

31. The system of claim 1, wherein the one or more Tn7 transposases comprises a transposase with activity of TnsA and TnsB.

32. The system of claim 1, further comprising a targeting moiety.

33. The system of claim 1, which comprises a plurality of guide molecules capable of directing binding of the guide-Cas protein complex to one or more target polynucleotides.

34. A system comprising one or more polynucleotides encoding:
a. one or more CRISPR-associated Tn7 transposases;
b. one or more Type I-B Cas proteins; and
c. a guide molecule capable of complexing with the Type I-B Cas protein and directing binding of the guide-Cas protein complex to a target polynucleotide.

35. The system of claim 34, further comprising a donor polynucleotide.

36. The system of claim 35, wherein the donor polynucleotide comprises a polynucleotide insert, a left element sequence, and a right element sequence.

37. The system of claim 34, comprising one or more polynucleotides or encoded products of the polynucleotides in one or more loci in Table 5.

38. The system of claim 34, wherein the one or more polynucleotides encode components (a)-(c) of any one of claims 1-33.

39. The system of claim 34, wherein the one or more Type I-B Cas proteins comprises Cas5, Cas6, Cas7, and/or Cas 8.

40. The system of claim 34, wherein the system comprises a first polynucleotide encoding a first Cas6 and a second polynucleotide encoding a second Cas6.

41. A vector comprising the one or more polynucleotides of any one of claims 34-40.

42. A engineered cell comprising the system of any one of claims 1-40, or the vector of claim 41.

43. The engineered cell of claim 42, wherein the cell produces and/or secretes an endogenous or non-endogenous biological product or chemical compound.

44. The engineered cell of claim 43, wherein the biological product is a protein or an RNA.

45. A cell line comprising the engineered cell of claim 42 and progeny thereof.

46. A plant or animal model comprising the engineered cell of claim 42 and progeny thereof.

47. A composition comprising the engineered cell of claim 42.

48. The composition of claim 47, formulated for use as a therapeutic.

49. A biological product or chemical compound produced by the engineered cell of claim 42.

50. An engineered cell or progeny thereof, the cell being engineered by use of the system of any one of claims 1-40.

51. The cell or progeny thereof of claim 50 that is isolated.

52. The cell or progeny thereof of claim 50 that is further used as a therapeutic.

53. The cell or progeny thereof of claim 50 from which a product is isolated.

54. A product produced by the cell or progeny thereof of claim 50.

55. The product of claim 54, wherein the product is a protein or an RNA.

56. The protein of claim 55, wherein the protein comprises a mutation.

57. A pharmaceutical composition for treatment of a disease or disorder, comprising the cell or progeny thereof of claim 50.

58. The pharmaceutical composition according to claim 57, wherein the treatment results in genetic changes in one or more cells.

59. The pharmaceutical composition according to claim 57, wherein the treatment results in correction of one or more defective genotypes.

60. The pharmaceutical composition according to claim 57, wherein the treatment results in improved phenotype.

61. The cell or progeny thereof of claim 50, wherein the cell comprises a mutation in a protein expressed from a gene comprising the target sequence.

62. The cell or progeny thereof of claim 61, wherein the cell comprises deletion of a genomic region comprising the target sequence.

63. The cell or progeny thereof of claim 61, wherein the cell comprises integration of an exogenous sequence by homology-directed repair.

64. The cell or progeny thereof of claim 61, wherein the cell comprises decreased transcription of a gene associated with the target sequence.

65. The or progeny thereof of claim 61, wherein the cell comprises increased transcription of a gene associated with the target sequence.

66. The product of claim 49, wherein the product is a mutated protein or product provided by a template.

67. A method of inserting a donor polynucleotide into a target polynucleotide in a cell, the method comprises introducing to the cell:
a. one or more CRISPR-associated Tn7 transposases or functional fragments thereof;
b. one or more Type I-B Cas proteins; and
c. a guide molecule capable of complexing with the Type I-B Cas protein and directing binding of the guide-Cas protein complex to a target polynucleotide.

68. The method of claim 67, wherein the donor polynucleotide:
a. introduces one or more mutations to the target polynucleotide;
b. corrects a premature stop codon in the target polynucleotide;
c. disrupts a splicing site;
d. restores a splicing site; or
e. a combination thereof.

69. The method of claim 68, wherein the one or more mutations introduced by the donor polynucleotide comprises substitutions, deletions, insertions, or a combination thereof.

70. The method of claim 68, wherein the one or more mutations causes a shift in an open reading frame on the target polynucleotide.

71. The method of claim 67, wherein the donor polynucleotide is between 100 bases and 30 kb in length.

72. The method of claim 67, wherein one or more of components (a), (b), and (c) is expressed from a nucleic acid operably linked to a regulatory sequence.

73. The method of claim 67, wherein one or more of components (a), (b), and (c) is introduced in a particle.

74. The method of claim 67, wherein the particle comprises a ribonucleoprotein (RNP).

75. The method of claim 67, wherein the cell is a prokaryotic cell.

76. The method of claim 67, wherein the cell is a eukaryotic cell.

77. The method of claim 67, wherein the cell is a mammalian cell, a cell of a non-human primate, or a human cell.

78. The method of claim 67, wherein the cell is a plant cell.

79. The method of claim 67, wherein insertion of the donor polynucleotide into the target polynucleotide in the cell results in:
a cell or population of cells comprising altered expression levels of one or more gene products;
a cell or population of cells that produces and/or secrete an endogenous or non-endogenous biological product or chemical compound.

80. The method of claim 67, wherein the donor polynucleotide is inserted to a site no more than 80, no more 90, no more 100, no more 200, or no more 300 bp 3′ of a PAM on the target polynucleotide.

81. The method of claim 67, wherein the donor polynucleotide is inserted to a site no more than 80, no more 90, no more 100, no more 200, or no more 300 bp 5′ of a PAM on the target polynucleotide.

82. The method of claim 67, wherein the target polynucleotide is linear, circular, or genomic DNA.


SEQUENCE LISTING (362 sequences)

SEQ ID NO: 1 - protein, 288 aa, Artificial Sequence
  Synthetic N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 4 aa, Artificial Sequence
  Synthetic
GGGS

SEQ ID NO: 4 - protein, 5 aa, Artificial Sequence
  Synthetic
GGGGS

SEQ ID NO: 5 - protein, 9 aa, Artificial Sequence
  Synthetic
GGSGGSGGS

SEQ ID NO: 6 - protein, 15 aa, Artificial Sequence
  Synthetic
GGGGSGGGGSGGGGS

SEQ ID NO: 7 - protein, 10 aa, Artificial Sequence
  Synthetic
GGGGSGGGGS

SEQ ID NO: 8 - protein, 20 aa, Artificial Sequence
  Synthetic
GGGGSGGGGSGGGGSGGGGS

SEQ ID NO: 9 - protein, 25 aa, Artificial Sequence
  Synthetic
GGGGSGGGGSGGGGSGGGGSGGGGS

SEQ ID NO: 10 - protein, 30 aa, Artificial Sequence
  Synthetic
GGGGSGGGGSGGGGSGGGGSGGGGSGGGGS

SEQ ID NO: 11 - protein, 35 aa, Artificial Sequence
  Synthetic
GGGGSGGGGSGGGGSGGGGSGGGGSGGGGSGGGGS

SEQ ID NO: 12 - protein, 40 aa, Artificial Sequence
  Synthetic
GGGGSGGGGSGGGGSGGGGSGGGGSGGGGSGGGGSGGGGS

SEQ ID NO: 13 - protein, 45 aa, Artificial Sequence
  Synthetic
GGGGSGGGGSGGGGSGGGGSGGGGSGGGGSGGGGSGGGGSGGGGS

SEQ ID NO: 14 - protein, 50 aa, Artificial Sequence
  Synthetic
GGGGSGGGGSGGGGSGGGGSGGGGSGGGGSGGGGSGGGGSGGGGSGGGGS

SEQ ID NO: 15 - protein, 55 aa, Artificial Sequence
  Synthetic
GGGGSGGGGSGGGGSGGGGSGGGGSGGGGSGGGGSGGGGSGGGGSGGGGSGGGGS

SEQ ID NO: 16 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 17 - protein, 32 aa, Artificial Sequence
  Synthetic
LEPGEKPYKCPECGKSFSQSGALTRHQRTHTR

SEQ ID NO: 18 - protein, 20 aa, Artificial Sequence
  Synthetic
PKKKRKVEASSPKKRKVEAS

SEQ ID NO: 19 - DNA, 9 bp, Artificial Sequence
  Synthetic
ggtggtagt

SEQ ID NO: 20 - DNA, 27 bp, Artificial Sequence
  Synthetic
ggtggtagtggagggagcggcggttca

SEQ ID NO: 21 - protein, 21 aa, Artificial Sequence
  Synthetic
GGSGGSGGSGGSGGSGGSGGS

SEQ ID NO: 22 - DNA, 72 bp, Artificial Sequence
  Synthetic
ggtggaggaggctctggtggaggcggtagcggaggcggagggtcgggtggtagtggagggagcggcggttca

SEQ ID NO: 23 - DNA, 48 bp, Artificial Sequence
  Synthetic
tcgggatctgagacgcctgggacctcggaatcggctacgcccgaaagt

SEQ ID NO: 24 - DNA, 192 bp, Artificial Sequence
  Synthetic
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 25 - DNA, 108 bp, Artificial Sequence
  Synthetic
ggtggttctgccggtggctccggttctggctccagcggtggcagctctggtgcgtccggcacgggtactgcgggtggcactggcagcggttccggtactggctctggc

SEQ ID NO: 26 - protein, 7 aa, Simian virus 40
PKKKRKV

SEQ ID NO: 27 - protein, 15 aa, Artificial Sequence
  Synthetic nucleoplasmin bipartite NLS
KRPAATKKAGQAKKK

SEQ ID NO: 28 - protein, 9 aa, Homo sapiens
PAAKRVKLD

SEQ ID NO: 29 - protein, 10 aa, Homo sapiens
RQRRNELKRS

SEQ ID NO: 30 - protein, 38 aa, Homo sapiens
NQSSNFGPMKGGNFGGRSSGPYGGGGQYFAKPRNQGGY

SEQ ID NO: 31 - protein, 42 aa, Homo sapiens
RMRIZFKNKGKDTAELRRRRVEVSVELRKAKKDEQILKRRNV

SEQ ID NO: 32 - protein, 8 aa, Homo sapiens
VSRKRPRP

SEQ ID NO: 33 - protein, 8 aa, Homo sapiens
PPKKARED

SEQ ID NO: 34 - protein, 8 aa, Homo sapiens
PQPKKKPL

SEQ ID NO: 35 - protein, 12 aa, Mus sp.
SALIKKKKKMAP

SEQ ID NO: 36 - protein, 5 aa, Influenza virus
DRLRR

SEQ ID NO: 37 - protein, 7 aa, Influenza virus
PKQKKRK

SEQ ID NO: 38 - protein, 10 aa, Hepatitis D virus
RKLKKKIKKL

SEQ ID NO: 39 - protein, 10 aa, Mus sp.
REKKKFLKRR

SEQ ID NO: 40 - protein, 20 aa, Homo sapiens
KRKGDEVDGVDEVAKKKSKK

SEQ ID NO: 41 - protein, 17 aa, Homo sapiens
RKCLQAGMNLEARKTKK

SEQ ID NO: 42 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - protein, 9 aa, Homo sapiens
IEVMYPPPY

SEQ ID NO: 44 - DNA, 7629 bp, Trichormus variabilis
  misc_feature (109)..(145): n is a, c, g, or t
ggggaattgtgagcggataacaattcccctgtagaaataattttgtttaactttaataaggagatataccatggtgctttaacattagatgtcgttaggcgttgagcannnnnnnnnnnnnnnnnnnnnnnnnnnnnnnnnnnnngtgctttaacattagatgtcgttaggcgttgagcagatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgattattgatagtaacttagttacgcagcttccctcagaatcatcttcaaagccaagagagcttttaatgtctcagtttccatatatggatttaacttttagccttacgggtgagacattacctttcgaccacggctatgaattattttctgcgatcgcccactttgaacccaaactacatacattagacacgttgggaattcagaccattgcaggtattcccaaggatggtgtaattaatctgacccaaaattctaggcttcgtattcggataccagtaaatcaggtacgtttggtttatccattggcgggtaagtcactgagaattggcaagcatacaattcgtttgggtattcctgacatctgtttgcttcaacctgcggagaaactgcgatcgcgcattgtggtaataagaggctatgaagaacctgaaagttttttagtcgtagcgcagcgtcagctagaacaactgggtatccaagcaattgctagcattcccaccaaagccaatggcaaaccaatacgtaggactatcaaaattaagcgatttacagtcgtgggttttggattagaagttataaatttaagcgatgaagactcactgaggttgcaaatacatggtgtaggaggaaagcaaaagatgggttgtggactcttcatgcctattaaggaaaggcaatagcaattactcgaaagcagatgatgaagagagcagataaccccaaaattcagcttaatctcggcgatcctagtatgactttattgcatcgtgctggggttgcaggactttggatgacgctcaaaaaactggaaaaaatctatccaacacctgctgaacgtattggtaatttaacttggttgctaacccctcactatatcaatcttgattgggaaggacaggatttcacagttttagactggttgctgaagcaatcatttcaaatcagcgatgagggtttaatttccctgacagggttagattctcaaaacatggatattcaaaatcaaataattatccatcaaggtatcacagccacttttacacaacataataaattttttaagtctgcgggtcaacaatctaagcaactaattattgatggtattcaggttagggttgattataagaaagctgcttcctatgctcatcaacattttgctaaacatttatgtgaccagtacggacaattgctaaaagagccaattggtatcaggggatggctctatccaggagcagtagttcgccattatgcttttaaagaacaaacagcatttgaagaaaaagcagaatatgctctagcgttgttgttcgctcctatggcttgtcaatattttgtgctgcgatcgcacaccgaaccatcacatacaaactatgttttggttattcctgaagtaattgatttagaactttatgcacattttttgtggtgtttgggtaacttagattataaacattttcacgtatctagcttaggagatgctggattaaaatttcttaattataaaacaacacaaatgcctacttccaattttttaaaacgatgtcaaataatcttttttagtacaaaagcttggtcagaacagcaaaaaactcgtgcagaaatagcaatcatagaattaaattcttcagttaattatctttataagcttagttgtatttgttttccagaatgtcaaattgttttatataaaaatcaacattatttgcttccaagtttaataaaaggaataatcgccaataatctagcaatgggtcggtcttggtgggctaattggatttcatttattaaaaataaaaaatcatttaaacaatttactgatgagtataaaggaattaataagatgattcaggactcagaatgggatatagaagctcaaaagctatttattcaagcttgtcatgaagcattaagaaaaatatatgccaaaatttatagcagaacaaaagaaggtgaatacgctcaaatagaacgagaaaatacacgcattttatctcagttaaaacgctgcaccaatactgacaatttccgaaaatttattgcagagttttggggaagggcagggcaaatagcaattctggaagaacattggatggaattacttccattgactacagggataggcaattggaaagtggccagggacttaacttttattgcaatggctagctacccaaagaataaaataacagaggtagaggtaaaaccatcacaaatatcggagctaaatactgaataaacttcatattttttgatttgattaagaacaaaatattaattgtttaggtaatggtggaatgtttcatctgtttggtaatattttgactagttatggaaccgccgctaataatcgtggtgaaaatgaaggtaataccactacgttacaaaaattaatttggaaaggtgaagttcactctactgtttcatctgaggcaattcgttgggcattgcgatattattggcaaaatgctggctatcctgtgaatagacgttgggatgaaaatgctcaaccagtacccgatcacatcttgcaagatcctaattttgatgatattcgcttcattgatgatgatgtcttgggatttatgcaagcggaagcagcaaaagttgaagcagcagttgaatccgaagttgaaatccagaactcaactcaagatgaggacaaaaaactttctcaaggtaaggagaaagaaactgcaaaacgtaagcgtaaggaaaaacctaaagggaaaataactactagacgaggtgtattagaagtcactcgtgcagtttcaacaattccttatgctggtgatcttactttcaatgctatgagtggaaagaaaggacgaacatctctttattctacagaagttcatgctactcgatatcaatatggcttcgcattaacacccaatagcctcaaagataaatctcgcattaatgctgtcctcgatagcttaatttctattggagaagtggcaggaaatcacgctcgttttctctatgatttttctcctgatagcatcatattacggtggacacacgatttctcacctcggttactttactgttttgaagaagatgaacttagaaatatatcaacaccggatttagtaaggcgggtagaatctggagatatagaccctaaagaattatggataggaggggcaatttctagcactctggaagatttaggggcaaatgtgtttcccggagtgaagacaacagttgaagcattaaaacaggtgattattgaagatttgcagttgtcgttaaggagttcatgatatgacgacaattgccttaaaagtagaagttcccatagcttgttttcggcagtctcgtgctagggaatatggcgaaacttaccctgtaccaccaccatcgacagtatatggaatgttactatctcttgttggagaagtggatagatacaaacactgcggtgtgaagctggcgatcgcacttctatctgaaccagaaaagtcaactgttattcgcacctttcaccgatttaagacgaagaatatccatgactctaaaaataataaacctgattatcaggagttattaactaatatagaatttatagtttgggtggatgctggagtagataaagctaaaccaaatttagtggaacgtttagcagaggcattgactaacccggcttctattaataggtttggtggattatgtttaggtgaaagtcgggatttagtgaatgatgttacccttttacctaaaaattactatgcagaatcaatgcgatggttaattcgagatgaatatgggttactaactttaccttactgggtagaccatgtaggttcgcggggaacgcgatggctacgttacgagattcagaaatgtccagtctttcaaccacctgaattatcttggacttcagttcaaagtaattagcctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 45 - DNA, 3959 bp, Trichormus variabilis
agcttgccattgcaactattggcttaccacatcgccgcccgtcgcggtttggatgtcgatcagcctcggaatttagcaaaatcagtgactgtggaataaataaatgtggaggaataattcggttcaacaatatataattcggttcaacaattaataaaacgattgaacaataacctctaaaggagtacgattgtaccaagtaaaagtgcgatcgcttccagaggttatttttatggttaggggtagaagggaatggcggggatccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttctagagtcccgggatatttacgttttgtaatcacgaatagattcttgattgcaagatgaattgcgatcgcatctttgtcgcacagcagaactttctcaaataattttgaacttatggaattactcaaaacctttatcagtaatggattcaaacaattgtttaaccgttttatttgagatttttactaaaaatcaagtttaccatagaggttaagctgaaattgttcaatcgttttattttatcgacaacggcagattaaatattcgaactgtaataattgtttaaccgaattctttgtctacaaatatttgtagagtagataagataatttctagatgtgactactatatcacatctaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 46 - DNA, 11096 bp, Trichormus variabilis
ggggaattgtgagcggataacaattcccctgtagaaataattttgtttaactttaataaggagatataccatggttaggggtagaagggaatggactcaagctaaatatgagcgatatataaaagaaggtcgtggccgaggaagtggtaagcactataaaccttggcttacaatccaagatgttccctctaaaggacgttcttctagaactcctggttggaaaacaaatcgagtacatcatttcttctcagaccatgaaaaacggctattttatttgtttgagtggtcagatatagtcacagatataagagagcaatttcctttagacaatctcgatctagccatgagtattgctacagacataggtataaagtaccctgtagatacacaaagtggcactcctcacgttttaacaagcgactttatgcttacagtcaaccgcgatgggaagcagatgcagattgcccggactgttaagccgtcaacagaactggagaaaaagcaagttaatgagaaattcgagatagaaagacaatattacttagcaaagggtattgactggggaattatcacagaaaaagaaattcccaggcttttagctgaaaatgttgagtgggttcattctgcttataggctagaagcaaattctgaaatggacactgcacaactgagtgaattgtcaacaattttaaaatctaagcttcaggaagaagatacaactataaatagagtaactactgctttagacaaagaaatgaatataaattccggtacatcactttatgtatttaaacatttaattgctaggaaagaaatccttgtggatctagtagcaataaaattatctgcttgtccgtcaactaaagttattgaaaaaataatattttagacccacatgataggtaaataatatggttaaagatttatttgtcaatgatttaatagagtggattgtataccaaaacactcatatagtcgaaagaattatttggatagatgaaggttacattattgcatttgttatagatattaatgctaagaaaggtctacctgaaccaaggaaaatatcagatattttagaagctataactgaaggtattgcttttaagcagcaacaagatccttgggcaagaattattcgggatgaaaatttgacagataaggaaaaggaatacagagataaagcttgggagataatttcatcattaattatagaagaaccatcaatatattatagaaattttagaggatcgatggttaaagaagcggttgaaaagtacaacgaagaaagggataaagataaattagtagaaaaaaccgtctatggttatttgagaagattttggcagagaggaaaaactataaatgctctcatacctgattttatcaattcatcaggtaagggcaagattcgaggacaatcgggcaagaagagaggaagaccaagaaaatatgcacatgacccagatattggtgaaggtgtaaatgttacagaagaagatagaaagatttttagagtatctataagcaaattttacaataatccaaaagaaaactacttaacaacagcctatgaattaatggtgaaagaatattacaaggaaggaattagatatgacgagaatggtgttcaaaaatctatattaaccccaactgataaaatacctacaattactcaattcaaatactggtatcaacttgaacaggatgatgttaccaaaactttaacatctcgaaaaggagccaagcgagtagctttagagaatagagcaattttgggaaactctaagatggaaactattggccctggttctagatatcaaattgatgccacagttgctgatgtatatttagtttcaaaatataaccgtgactggataattggtagaccagttatctatgtaattattgatgtttttagtagaatgattacaggtgtttatgtcggtttagaaggcccatcttggataggagcaatgatggcactagccaatgtagctacagataaaaccctgttttgccaggaatataacattaatattactgaaaatgaatggccttgcaatcaaatgccagatgcaattttaggagataggggagaattagcaggtatgacagtagaaacattaatccctaatctttatgttcgtattgaaaatgcagcttcctatagagcagattggaaagggttagtagaaagacactttcgtataattcatgaatatgtaaaaccttttttacctggttatgtggatacggattttaggcaaagaggtgcaagggattataggcttgatggcaaacttgatatagatcaatttactgagattattattcgtttaattgttcaccataacaataaacctcttactgaatatgaaagagatgaagcaatgattgctgatgatacccctcctatacctagagagttgtggaagtggggtatcgccaatcgttctggcagactcagaactttttctgaagatattgtcaaattgaacttgatgcctactgagaaagcaactattactgctaaaggcatcaaaatgagaggtaaggagatgtactatacctgtgatagtgctaaaaatgagcaatggtttgaaaaagctagaagtaatctcctttctaagtcagaaaaatcattgattgtatcttatgacatcagaaagccaaacttcatttatcttccatctagtgatggtagagattttgaaaaatgttttcttattgacttagaagggcggtatgctaataaaaactttcatgatattgaatatctgcttgcttatgaagaattgcaaagacagaaaagccaaggtaaacagctacaagaaaaggtagatttaatagctgatattgaaagcatagtagatagagcagaaaaaatgactgatgcttctcaatatcataatttaagtaagagcaagaaagtttctggaataagagataatagggcttttgaaaaggctaaacgacgagagaatgaaggatttgaattagcaattacagataatcagagcagttttcaggttgcagagagtacagcagaaacctccgaacagccagaatcactacagccaaaccatctgaatcttcttaagaaaaagcgacaggagcgaaaacgtggacagaattgagagtgaactggttaaaattcctaatggagaactagcagttattgccagatatatagatcaaaaactgccagaatacaacactaatccattaattcaggcattacctcctatcctatcagcagcagaatttatagataaagtagccagagaaccaaattttgatgaacaagagagagagctagaaccacattatagatttcattgtattgagaggttatctagatattttgaccctcaaaataaaactcttgaattacaaaaagttatttgtacgcttatcatgacaggctatctgtcaaggaaccttttaaaacctgaatatgcaaatcgctctaggcaaatttataatgcaataggtaatggaggaggtaaaaatttagaaaactatgttaatcttcctacatctgcgtccggcttaactctaattggcccatcaggtattggaaagactacaaatttcaaaaatattcttgagctttatccccaaataatttttcatcctgattatagtgtttaccaaatagtttggctgcgggtagattgccctaaaactttaaaggggttatgtatagatacttttgcaggtatcgataaattgttaggaactaatttattaaagaaatttaacccagcacgcacatctgaagattatctattggctcaacttgcacaaacagcacacactcatcatctaggagttttggtgattgatgaaatacaaaatttagcaactgcaaaaagaaatagagacgaaatattaaattttttagtaaagctcgataatactattggaattccagttattcgtgtaggtacgaatgaagccattagttttcttcaaggtaatcttagaaatgctagaagaaatactggagaaggaagtagaatgtgggatagaatggtaaataatgatgaatggtgttactttttagaaggaatatgggaataccaatggacaaaaacacctgttcatttctcggatgaaataaaagaagttttttatgatgaaagccagggaattattgatattgctataaaactttacaaaatggttcaatggagagcgatttcaataggtgaccaagaaattattacggttgatctgattcgtcaagtagctaaagaaggattgtatttagtaaagccaatgctagatgcaattaggttaggggacagagagtggatgattaaatataaggatattgctcctctcgatattacggaatatcacaatagatgtgtatctgagattgatgctaaagatttgacagagattaagagattagctaaaaagcagcagaatcagcagaaagtatctccgaagctcaattatgtaattagtgagctgttgaaattggaagtagaatctggtaaagcaaaggaatgtgcagaacaaattgtttatagtcatgaagatccagacattccaattccatctttagttaataaggcatatgctcttgctttgcaaggatgtcaagtaaatgagagtcttgctaaaacaagtaccaggaaagctaaattaaagacaaagtaccaagagaatgatattcggcgaattgtcgagaatgccaagaaagacaaaaaatctgcatacgaggatttgaaattagcaggtataatcaaaaatccagttgaagattttatcaatatttagcttctaaatgcttagttttttcccaactctgtatcccgatgaacttttatatagcactttagctagataccatattcggagtggaaataaaagtttcaagcaaactgacttagagctattcggttacagttcccaacaaatatgcaaaattaccttaactaataatttaaattatttagtgaaaaacttacctttacgctcccagcaaaaagttgagaatttactacaaaatcatactttatattccttttatgcaactttattaatgccgcaggaggcttggctgctgaaaaactcaatgagtaataaattcagtgggtctatactagattttgctaaagtggctaataattctagtgatgattcaaaaaaatttttaaaattttgccctgaatgtttagagaaagatacccaaaagtatggtgaaccttactggcatagaatacatcaagtccctggcattttagtttgtccaattcatcgtgttgttttacacaatagctgtatagaagttgaatcgaaaggcatacattatcatgctgccagcatagataattgtttattagcaaataatgtgaaaaaattgacaaataaaactgtagaaacactttcaatactggcacatgatattaattggctaattcatagcagttttgcctttcaaggtttggcttggcttcgcgctcagtaccagcgttacttattggataaaggactgctaaaaacctttgctggtggcaagtttaagtttaatgattgtggttttgcccagcttatatttgagttttatggtcaagaatccttagaagccctcaatccaaagctgacccaaaatcaaggtaaatatttttctcactgcttgtttggatgcgacttaaacccagcaattgaccgcataactcatatcttaatgataaaatttttagcaaattctatagaagagttctttcagtcataacttaagtcgaacagaaagtaatcgtattgtacacggccgcataatcgaaattaatacgactcactataggggaattgtgagcggataacaattccccatcttagtatattagttaagtataagaaggagatatacatggtgagttttttccctcaaccttatccagatgaaattttatatagtgttattgctcgttatcacattagaagtggcaatacgagtccaaaaattacattacaagatttatttaagtcacagactactgtggcgacagtagatttaccatcgaaccttagtaatttaattaaaaacctacaatttatctcaaattaccaagttgaagagttgatatataaacatactctctaccctttatatagcccttttttgccagcaaacaggtctagtcaggtactggaatctatgaaaggagattatgggggagatattcatacaagagccggaattatggcaagctcaattactatgccgaaatatttgcggttttgtccaacttgctgggaagaagacttaaaaaattatggcgaaccttactggcatcgcatccatcaaataccagggattttagtttgtccatttcatggtgaagttttgcaagatagcctagctcctttacatggttttaataagcatgaatattatgcagctagtacagacaactgttctattactaaagaacccagtattcttagccctgacacactgaaaaaattactaatattagccaaagatttttggtggttactcaatagtgaaataccttgccaagagccacagtggtttcgcaaacagtacacaaatctgatgattgagcagggttttgcaactccaactggtcgaatttaccagaacaaactactagataattttttgttcttttatggtcaagaaatgctcagtgctgttgattcaatggtaaatcataatgattcaaacaactgggttactagcatcgtcaggaaatataggaaatctttccatcctcttagacatctgctaatgatgcgttttttgaaaacatcagtagaggaattttttcaaaaagatactgaatacaagccttttggcgaacccccctggttgtgtctcaatgctgccgcagaacattatctacaaccagtgattaccaatttagtcattactttatgctgcgatacaaaaaagcctgtcggtactttttcttgtgcttgcggaatggtttattgccggactgggcctgatgaaacggatgaagacaaagttcgcattggtaaagtcaaggcattcggtcaaatatgggagcaaaagctgaaagaattggtggaagttgaaaagttgggtttgcgggagacagccagaagattaaatgttgatgccagaactatcaaacgttatgtatctcgtctaaaattgacaacttattggcagactagaacagaaaatgactctgtaggcttacagggagtcccagaaatagatcccaattttgcaactgaactcaaattaaaacacaggcaaaattggatggctttacaagtacagaaccctgaagcttcaaagacaactctgagaaaactagtaccagccacctatatttggctatacaggaatgacagagaatggctcaatcaaaactcacccactttacaagttgccgttccttccgttgtgagagttgattggcatgagcgagatcagcaaattctcgctcaggttcaagatgcagtgcgatcgctacttaaggctgatataccagagaaaatttctattagtagggtagggaaaactatcggcttacttgcattactcgaaaaacatctcgaccagatgcctttaacaaaggcttatttggaatcagtcgttgaaactgtagaagaatttcagatgcgccggatcaagtgggcaatcaagctgcttgatgaatgtggagaagaaattatgcgctggaaggtagtcagagttgctgctttgcgagaggattgctcccaaagagtcagtgcatttctggaaagtgaactggagaaagcatacaagaaagagaggtgattaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 47 - DNA, 300 bp, Trichormus variabilis
ccgggatatttacgttttgtaatcacgaatagattcttgattgcaagatgaattgcgatcgcatctttgtcgcacagcagaactttctcaaataattttgaacttatggaattactcaaaacctttatcagtaatggattcaaacaattgtttaaccgttttatttgagatttttactaaaaatcaagtttaccatagaggttaagctgaaattgttcaatcgttttattttatcgacaacggcagattaaatattcgaactgtaataattgtttaaccgaattctttgtctacaaatat

SEQ ID NO: 48 - DNA, 152 bp, Trichormus variabilis
tgtggaggaataattcggttcaacaatatataattcggttcaacaattaataaaacgattgaacaataacctctaaaggagtacgattgtaccaagtaaaagtgcgatcgcttccagaggttatttttatggttaggggtagaagggaatgg

SEQ ID NO: 49 - DNA, 37 bp, Trichormus variabilis
tgtctcctgggaggattcataaagcattgtttgttgg

SEQ ID NO: 50 - DNA, 37 bp, Trichormus variabilis
gtctcctgggaggattcataaagcattgtttgttggc

SEQ ID NO: 51 - DNA, 37 bp, Trichormus variabilis
gattcataaagcattgtttgttggctacgagaagcaa

SEQ ID NO: 52 - DNA, 37 bp, Trichormus variabilis
gagaagcaaaataggacaaacaggtgacagttatatg

SEQ ID NO: 53 - DNA, 37 bp, Trichormus variabilis
aagcattgtttgttggctacgagaagcaaaataggac

SEQ ID NO: 54 - DNA, 37 bp, Trichormus variabilis
cataaagcattgtttgttggctacgagaagcaaaata

SEQ ID NO: 55 - DNA, 37 bp, Trichormus variabilis
tctcctgggaggattcataaagcattgtttgttggct

SEQ ID NO: 56 - DNA, 37 bp, Trichormus variabilis
tcctgggaggattcataaagcattgtttgttggctac

SEQ ID NO: 57 - DNA, 37 bp, Trichormus variabilis
ggattcataaagcattgtttgttggctacgagaagca

SEQ ID NO: 58 - DNA, 37 bp, Trichormus variabilis
ctcctgggaggattcataaagcattgtttgttggcta

SEQ ID NO: 59 - DNA, 37 bp, Trichormus variabilis
gaggattcataaagcattgtttgttggctacgagaag

SEQ ID NO: 60 - DNA, 37 bp, Trichormus variabilis
attgtttgttggctacgagaagcaaaataggacaaac

SEQ ID NO: 61 - DNA, 37 bp, Trichormus variabilis
taaagcattgtttgttggctacgagaagcaaaatagg

SEQ ID NO: 62 - DNA, 37 bp, Trichormus variabilis
cctgggaggattcataaagcattgtttgttggctacg

SEQ ID NO: 63 - DNA, 37 bp, Trichormus variabilis
agaagcaaaataggacaaacaggtgacagttatatgt

SEQ ID NO: 64 - DNA, 37 bp, Trichormus variabilis
tgggaggattcataaagcattgtttgttggctacgag

SEQ ID NO: 65 - DNA, 40895 bp, Anabaena sp.
ggttttgaattcaagtaacgcttcatatatgtttacaaatcggctaaatctctgattgggtgatgaggagaaatacgaggcggaaatgtggctattttataggaacagagagagattgccgaaaagtgaattttgagaagtgcctattaaattgtttaaagttatgtagccttttacttacgccgtataagccactgcggtaaactatgcgttgattatgattctttcacagataaagaacactggaaaggagccgttttttcaatgtctcataccgtaaaaatctacgatacctgcattggctgcacccaatgtgtccgcgcttgcccgactgacgttctggagatggttccttgggatggctgcaaagctgctcaagttgcttcttccccccgtacagaagactgtgtaggctgcaaacgttgtgaaactgcttgccccaccgactttttgagcatccgggtttacctgggcgcagaaacgactcgcagtatgggtctagcttactaaggaattaactgctaattcctgatttagttgaccattcagtgcctcgatagcaaacaactttagcattgtagggtgggcggcgatcgctcatcctacaatgtcaaactggaaaaatatgaatatacttcttgacacaggggctaacagctcctttttttgtggaactcgatatttagatcagcatctatacttgatgtaattatcctgaacaggtggtgtgagtaatgtgtggcatcgttgggtatataggcactcaaacagcgacagaaattttattagcagggttagaaaaactagagtatcggggatatgactccgccggaattgccacggtttgggaaggagaggttaattgtgtccgcgctaaagggaaactctacaatcttcgttctaagctggaacagtcagcaacaccagcccaaataggcatcggacacacacgctgggcaactcatggtaaaccagaggaacataacgcccatccgcatttagacacagctatgcgggttgctgttgttcaaaatggaattattgaaaactaccgcgagttacgcgaggaactgaaacagaaggggcatcagttccgttcgcaaaccgatacagaagtgattccccatctaattgcagaatatttacagcatcttccctcaccttctcaccccctcaccccctctgactttttagaggcagtgcgccaagctgccaatcatttagaaggggcatttgcgatcgccgtcatttcggctgactaccccgacgagttggttgtggtgcgtcagcaagcccccttagtgataggatttggtcaaggtgagtttttctgtgcttctgataccccggcaattgtcgcctatacccgcgccgtactccccctagagaatggcgaaatagcccgcctcacacctttgggagtggaaatttacaactttgctggcgacaggttgaaaagacaaccccgaatgctcaacttgaatcccacgatggtagaaaagcagggattcaaacacttcatgctcaaagaaatttatgagcaaccgggagtagtcagagctagtttagaagcatattttgatcatgataccgttaacctagacttaccacaggatttttacgcagatttagagcaaattcacatcgtcgcctgtggtacaagttggcacgctgccttagtgggtaaacacttactcgaacaactagcgggaatttctacccaggtacattacgcttctgagtatcgctatgccccatcacccctgacagccaataccttaattattggtgtcactcagtcaggggaaacggctgataccctagcggcgttagcaatggaaaaagaacgccgccaaggaaaagaagcgaaatatcaggcgcgacttttggggattaccaaccgcccagaaagtagcctcggtttgatggttccccatattattaataccctagccggaattgagattggggtagcggcgacaaaaacctttattgcccagttgatggcgttttatgccttagcattggatttagcggctcgtcgccagacactaccaccggaaaaactcacagaaattattcaaggcttgcgccagattcccaaggaaattgaggcgacattagaacgtcaggaagaaatcaccgaacacttagcccatgaatttgcggaaaccaaagattttatctttttgggtagagggattaacttcccgatagcattagaaggggcgttgaaattaaaagaaatcagttatattcacgctgaaggttatccggctggggaaatgaaacacggcccgatagctttgttagatgctaaagttccggtagtagcgatcgcagttcctggtactgtgtatgaaaaggtgatttctaacgctcaagaagccaaagccagagattcccggttaattggggtgactcctgtgacagatggcgaagccgcagaaatctttaacgatttactcccagtgtcatcagtggatgaattattatctcccatcctgacagtcgtacctttgcaacttttggcttatcatattgcagcaagaaggggtttagacgttgaccagcctcggaatttggcgaagtcggtaactgtggagtaaaataatactttaaataagtataattgtacaacaataaaaaagtcgtataacaaataaaaaagttgtacaatatataaaaaagttgtacaatatatcctgtgggtgcatatgcaagcatctacaggagtttttatggctagaagtaagcaagattggacgcaagccaagtttgagcgttactgtaaggaaggtcgtggtcagggcagtgggaaaaactatcagccctggatcaagattcaggactttccctccaagggtcgtgtttccagaccaccaggttggaaaacaaaccgagaacatcatctcttttctgataacgagaagcgactgttttacgtatttgagtggtcagatgcgatcgttgatattagagagcagtttcctctacttgatctggacttagcaattaatattgcggatgaaatgggtattaactatcccaaagatccgcaaagcaatacgccatatgttttagccacggattttatgctttctgtccagcaaggtaagaatatagttcagaaagcacgaacatttaaactaaccaaggatttgggaagtaagtcggtagctgaaaaatttgagttagaaaaacgttactttgctgccaaaggtattgattggggaataatcacagaaaaagaagtccctaaacaattagctgagaatgttgaatggattcatactgcttataaattagaagaaaatgcagatataaatcttgaagaattacgtaacatcgcaaatattttaaagtttagacttcaggaaagtgatgcaagtatcaataaaataacaacggttttagataaggagatgaacatagagtctggcacatctctctatctttttagacaccttattgctcggaaagaaatcattatggatatgctggtaaataaaatatctagttgtccatcttctaaagaaattcaaaaaataattttttaagctaggacgaggtactttatatgattaatgatttgtttgtaaatgacctgattgaatggattgatgaatccggtaataactttatagaacgagttctctggattgatgaaggatatattattgcctttgtttatgatataaatgctaaaacaggatttccagaggctaaaaaagtttcagagattagagaagctatttctgatggtcgtgcattgaagcttaaatcagacccgtgggcaagaatcgtaagagatgaagatttgtcagagaaagaaaaagaacttagagatagggcttggcaaataatttcatttatcgttactcaagaaccctcaatatactatcgggattatagaggtactttagttcagcaggttattgaaaaatataatgcaggtagaactgaaggtgaattagtaccaataactgtttataaatatttaagaagattttggcagaggggtaaaaacaagaatgctctcttgcctgattatgcaaattctggtggtaaaggaaagcctaaatcttctggagacaagaagagaggaagacctagaaaatacgcacaagttccagaaattggtgttggaagaaatgttacagaagaagataaaagaatatttagatttgcaactgctaaattctataataattccaaagaaaatttcttaacgactgcttatgatttaatgattaaagaatattacgctgaagatttttattacgatgataatggagttaagaaaagtattttaattcctccagataaacgacctacatttacccaatttaaatactggtacgaagtagaacaaactgatatcagaaaaactatgatatctcgtaagggttcgagaaaatatgccttagaaaatcgagccattaccggaacttcccaaatggaaactattgggccagggtctagatatcaaattgatgctacgattgcagatgtatacttagtatccctttacaaccgcaactggattattggtagacctgttatatatgtggtgattgatgtttttagtcgcatgataacaggggtttacattggattggaagggccatcttggacaggagcaatgatggctttagccaatgcagctacggagaaggtcaagttttgtcgggagtatggtattgagatttctgaagatgagtggccatgcaagcatattcctgataccattttaggcgacagaggtgaattagcagggatgacagtagaaacattaatccctaatttaaatgttcgtattgaaaatactgctccttatcgagcagattggaaaggattagtggaaagacaatttcgcattattcatgggtatgtcaaaccatttgttcctggttatatagatactgactttagacagagaggaggacatgattatcgccttgatggtaggcttaacatagatgaatttactaaaatagttatatttttgattctccagcataacaatcatgattatctaattgattatgctagagatgaaatgatgattaccgatgatgttaatcctatacctagagaattatggcaatggggaattgccaatcgttctggtagactgagaacttttccagaagatattgtcaagctaaatttaatgcccacagaaaaagcaacaattactgctcgtggtatcaaatttaaaggaatgtactacacttgcgagaaagcaaggaaggaattttggtttgaaaaatctagaagtagtttattatctaagtcagagaaatcattgaatatatcatatgatattcgacagccaaactttatttatttacgttccccaaacggtagagaatttgaaaaaggttggcttttagaatcgcaacaaagatatttagataaaaacttttatgacattgattatttattagcatatgaaaaattgcaaaatcaaaaacatcaaggtactcggttacaggcaaaagctgatctgatagccaatatagaaagtattgttagtcaggctaaagaagagactgaggcagttttagatgatactataagtaatagacaaaaagttagtggcattcgacagaatagagcagacgaaaaaaatgctcgacgtaaaaatgaagcatttgaactagcaaaagcagaaactcccaatactgataatactaagaatctaataaacactcaatctgaagattccgaggattcaaaactattacagcctgaccatatggatttactcaaacggaaaagacaggagcgaaaacgtggaaagtaatgaggaagaagttgttagtattcctaatggaaagtcagcagttattgccagatacatagaccaaaaacttccagaatataacgctaatccattgattcaggcactacctcctatcctctcagcagaagaatttatcgataaagtaacgagaacaccagattttgatgaacaagagagggagctagaagcgcactacaggtttcattgcattgagaggctatctagatattttgatcctcaaaataaaactgttgaactacagaaggtcatttgtgcgctaattatgacgggctatttagctcggaatattctgaaacctgaatatgcaagtcgctctaggcaaatttataatgcaattaaagatggaggagggaaaaacttagaaaactatgttaatcttcctacatctgcttccggcctaaccctaattggcccatcagggatgggaaagtctacgaatttccaaaatattcttaatctttatactcaagtaattcttcatcctgagtatagtgtttaccaaatagtttggttgaaggttgactgccctcatgctggttctttaaagggtctgtgtacggatatttttcttgctgttgacaggctattaggtactaactactttaaaaagtttggttcctcacgcaactctgaagattatatgctggctcaagttgcccaaattgcccatactcatcatttaggggttttagttatagatgagatgcagaatctggttacagcaagaagacgtagctcagatatgttgaattttttagtaaaaatggacaatactgtaggaattccagtaattcgagtaggtactaatgaggcttttcctattcttcaaggaaattttagaaatgccaggagagggacaggagaaggcagtgtaatttgggacagaatggtaaatgatgatgaatggtatttcttcatggaaggaatgtgggaataccagtggacaaaaactcctgttcctttctcggatgaaataaacgaggttttttatgacgaaagccagggaattattgatattgctgtcaaactttacaaaatggttcaatggagagcgatttcaataggtggtaaagaaattattactattgagttaattcgtcaagctgctaaagacggactatatttagtcaagccaatgcttgatgcaattaggtcaggggacaaggagtggatgattaagtataaagatattgcgcccctagatactacagaatatcaaaataaatgtctatctgagcttgattccaaggatttgaaagaaattcgcagattggcaagaaagcaacagaatcagcagaaagtatcgccgaagctaaattatgtaattcttgagcttttgaacttggaagtagaacctgctaaagcaaaggaatgtgcagaacaagtaattgtttctagtgatgaagatgccgaaattccatctttagtcaaaaaggcatatgctcttgctttgcaaggagggcaagtaaatgagagcctgactaagacaaatactaagaaagctaagttaaagcccaattatcaagaaaatgatattcgacgaattgttgaaaatgccaataaggagaaaaaatctgcatacgaggatttgaaattggcaggcataatcaaaaatccagttgaagattttataaatatttagcttctcaatgcttagtttcttcccaactctgtatcccgatgaactgttatatagcactttagctagataccacattcggagtggaaataaaagtttcaagcaaactgacttagagctattcggttacagttctcaacaagtatgcaaaattactttaactaataatttaaattatttagtgaaaaacttacctttacgctcccagcaaaaagttgagaatttgctacaaaatcatacgttatatcccttttacgcaacttttttaatgccacaggaggcttggttactaaaaaactcaattagtaaaaaactcagtggctccatactagatgttgctaaggtggctactaattctattggtgattcaaaaaaatttttaaaattttgtcctgaatgtttagaaaaagatacccaaaaatatggcgaaccttactggcatagaatacatcaagtacctggcattttagtttgtccaactcatggtatcgttttacaggatagttgtgtaaaagttgaatcgaaaggcgtacattatcatgctgcgagcgtagataattgtttagtagcctgcaaggtcaaaaatttgacaaataacacagtaaaaacactcttcacactggtgaatgatattaattggctaattcatagcaattttgcctttcaaggtttggcttggcttcgcgctcagtaccagcgttacttagtggataaaggtctgctacaaacctttcctggtggcaagtttaagtttgatgagtatggttttgcccagcttatatttgagttttatggtcaagaatccttagaagccatcaatcccaagttgactcaacatcaaggtaaatacttttctcactgcttgtttggatgcgacttaagcccagcaattgaccggataactcatatcttattgataaaatttttagcaaattctatagaagagttttttcagtaataatctgcatataaaaactgttaaatacattaatataaagtaataaataattaagaaaattatctttataggttggtatttcaagtattctcagattcgtgagtaaggtaaagtaagtaatatcatatttatttcacaccacgttgcatctactgaaatttggcatctaaaaatgattgttgatggcaacttacttaggcagattccctcagaatcaccaccaaagccaagagagcttttaatgtctcagttaccatatatggatttaacttttaaccttattggtgagacattacctttcgaccacggctatgagctattttctgcgatcgcccactttgaacccaaacttcatacattagacacgctgggaattcacaccattgcaggtattcccaaggatggtgtaattaatctgacccaaaattctaggcttcgtgttcggataccagtaaatcaggtacatttagtttacccattggcgggtaagtccttgagaattggtaagcatacaattcgtttgggtattcccgacatatgtttgcttcaacctgccgagcaattgcgatcgcgcattgtggtaataagaggtcatgaagaacctgaaacttttttagtcgtagcgcagcgtcagctagaacagctgggtatccaagcaaccgctagcattcccaccaaagccaatggcaaaccaatacgtaaaactatcaaaattaagcgatttacagtcgtgggttttggattagaagttacaaatttaagcaatgaagactcactgacgttgcaaatacatggtgtaggaggaaagcaaaagatgggttgtggactcttcatgcccattcaagaaaggcaatagcaattactcgaaagcagataatgaagacagcagctaaccccaaaattcagcttaatctcggcgatcctagtatgaccttattgcatcgtgctggggtggcaggactttggatgacgctcaaaaaactggaaaaaatctatccaactgctgctcaacgtattggtaattttacttggttgctaacccctagtaatatcagtctttattgggaaggacaggatttcacagttttagactggttgctgaagcattcatttcagatcagtgatgagggtttaatttccctaacagggttagattctcaaaacatggatattcaaacgcaaataattatccaccaaggtatcacagcaacttttatacaacataataaattttttaagtctgcgggtcaacaatctaagcaactaattattgatggtattcaactcagggttgattataagaaagctgcttcctatgctcatcaacattttgctaaacatttatgtgaccagtacggacaattgctacaagagccaattggtataagggggtggctttatccaggagcagtagttcgccattatgcttttaaagaacaaaccgcatttgaagaaaaagcagaatatgctctagcgttgttgttcgctcctgtggcttgtcaatattttgtgctgcgatcgcacaccgaaccatcacatacaaactatgttctggttgttcctgaagtaattgatttagaactttatgcccagtatttgtggtatttgagtaaattagattataaacattttcacgtatctagcttaggagatgctggattaaaatttattacttatgaagcaaaacaactagataatttcaattttatcaaacgatgtcaagtaatctcctttagtacaaaagcttggtcagagcggcaaaaaactcgtgcagaaatagcaatcatagaattaaattcttcagttagttatctctataaacttagttgtatttgttttcctaattgccaagttgttatatataaaaatcaacattttttgcttccaagtttaatgaaaggaataattgccaataatctagcaatgggttggccttggtgggctaattggatctcagttattaaaaataataaatcatttaaacaatttactgatgagtatataggaattaataaaatgattcaggactcagaatgggacatagaagctcaaaaattatttattcaagcttgtcatgaagcattaaggaaaatatacgccaagatttatagcagaatgaaagaaggtggatatgctcaaatagaacgagaaaatacacgcattttatctcagctaaaacgctgcaccaatgctgacaatttcagaaaatttattgcagagttttgggggagggcagggcaaatgacaattctggaggaacattgggtggaattactaccattgactacagggataggcaattggaaagtagccagggacttaacttttattgcaatggctagctatccaaagaataaaataacagaggtataggtaaaatcatcacaactatttgagctaaatcgtgagtaaacgttagattttttgatttgattaagcacaaaatattaattgtttaggtaattgttgaatgtttcatctgttcggtaacattttgactacttatggaaccgccgctaataatcgtggtgaaaatgaaggtaacacaactactttacaaaaattaatttggaaaggtgaagttcactctaccgtttcatctgaggcaattcgttgggcattgcgatatcattggcaaaatgctggctatcctgtgaatagacgttgggatgagaatgctcaaccagtagcggatcacatctgggaagatcctaattttgatgatattcgcttcattgatgatgatgtgttgggatttatgcaagcggaagcggcaaaagttgaagcagcagatgaatccgaagttgaaatccagaactcaactcaagatgagaaccaaacaacctctcaaggtgaggagaaaaaaacctcacaacgtaagcgcaagcaaagacctaaagggagaataagtgctagacgaggtgtattagaagtcactcgtgcagtatcaacaattccttatgctggtgatattactttcaattctatgagtggaaagaaaggacgaacatctctttatgctacagaagttcacgctactcggtatcaatatggctttgcattaacacccaatagactcaaagataaatctcgcattcatgctgtcttagatggcttaatttcgattggagacgtggcaggaaatcacgctcgtttcttttatgatttttctcccgatagcataatattacggtggacacatgatttctcacctcgcttactttactgctttgaagaagatgaacttagaaatatatcaacacccgatttagtgcgacgggtagaagctggagatatagaccctaaagaattatggataggaggggcaattgctagcactctagaaaatttaggggccaatctttttcccggagtgaaggcaacagttgaagcattaaaacaggtgattattgaagatttgcagttgtcgttaaggagttcatgatatgacgacaattgttgtaaaagtagaagttcccatagcttgttttcgccagtctcatgctagggaatacgctgaaacctaccctgtaccaccgccatcgacagtatatggaatgttactatctctggttggagaagttgatagatacaaacactgcggcgttaagctggcgatcgcacttctatctaaaccagaaaagtcaactgttattcgtacctgccaccgatttaaaacgaagaatatccatgaatctaaaaataataaacctgattaccaggatttactaactaatattgagtttgtagtttgggtagatgctggggcagataaagccaaaccaaatttagcgcaacgtttagaagaggcatttactaacccagcttctattaataggtttggtggattatgtttaggtgaaagtcgggatttagtgaatgatgttagcctgttacctgaaaattactatgcagaatcaatgcgatggttaattcgagatgattatgggttactaaccttaccttattgggtagatcatgtaggttcacggggaacgcgatggctgtgttacgaaattcaggaatttcaagtatttcaaccacccgaattatcttggacttcaattcaaagtgattagagggcattatttacttttgcaagcatctttgcttgggcaatttaaataccacaataaataatcttgtcttcttgtttcacaagcattttagggatttcatatactttcaatgtgcttgcaatttcctggaacaccttcaaaacaagggttagagctttagcaactttgcttttgctatgaatagctattaatttttgcaacttgactaatcctaattggaaggtgcttgtaaaattagtacagagctttggctagtcaacaggtttgatgatcgccgtgctttaacattagatgtcgttaggcgttgagcagtgattaacctgaaggcttgctccgggatgttgtaggtgctttaacattagatgtcgttaggcgttgagcagcaccggtaccgcgagtagtcgcaccaatattcatatgtgctttaacattagatgtcgttaggcgttgagcagttgaatgcgaagttatcggcagatgttttgttgttgtgctttaacattagatgtcgttagacgttgagcagtgaccattggtgtatccatcaatcctgactgctgcgtgctttaacattagatgtcgttaggcgttatctacttttgatacaaatacttttgaaatacatacaaaactagctctgccaaatatctccttttcaagacatttttgcgattattttttcccactgctcaatctggttctgtagttgtaattgccgatcgcccaaatcttgactatcatcaacagacaaatcctgaccgctcaatcgctcacgatagagagcaatcagttcggttagggcttttttattgcttgctgatcctcctgggtcagagaactaacattgactggaatcaactttctgccaattctctctacaaactcagaccgacttaaatccatactgtcggcgatcgcatctaatccatcaatcccggtaggagttagggctaggttaactcgtttcttcacctcatcgtacagttctggtacgtctttttgacctttttgccctttcttacccattgccgtctagtttttgctttcctcccaatcatagtaaacacatttagcacttaagttaaacattaaaattttttccaatactttaggtattgtgtaatccctaatttagttagataatacaggcattgtgtaatgcctaagcaggcatctcgtaaggatatgagtaactaattgcaccgtttgattcctcaacatctcaacggcttaattttcatgggcaagcattaattgcttcgtggccaattcagattaatcaaaatctgccagatgatttaggcatttttcaaaactatatggttagtttcttccctaaaccttatccagatgaaattttatatagcgtcattgctcgttatcacattagaagtggcaatacgagtcccaaaattacacttcaagagttattcaattcacaaactactgtcgcaacagtagatttaccatcgaacctgaataatttaattcaaaacctacaatttatctcaaattaccaagttgaagatttgatatataaacatactctctatcctttatatagcccttttttacctgcaaacaggtctagtcaggtactggaatctatgaaaggagattatgggggagatattcatacaagagccggaattatggcaagctcaattactatgccaaaatattttcggttttgtccaacttgctgggaagaagacttaaaaaattatggcgaaccttactggcatcgcatccatcaaataccaggggttttagtttgtccatttcatgatgaagttttgcaagatagcctagctcctttacacggttttaataagcatgaatattatgcagctagtacagacaaatgttctattactaaagaaccaagtatttttagccctgacacgttgaaaaaattactggtattagctaaagatttttggtggttactcaatagtgaaataccttgccaagaaccacagtggtttcgcaaacagtacaccaatctgatgattgagcagggtcttgcaactgcaactggacgaatttaccagaaaaaactactagataattttttgtttttttatggtcgagaaatgctcagtgccgttaattcaatagtcactcataatgattcaagcaactgggttactagcattgtcaggaaacataggaaatctttccatcctcttagacatctgctgatgatgcgttttttgaaaacatcagtagaggaatttttcaagcaagataataaatacaagccttttggtgaacccccgtggttgtgtctcaatgctgccgcagagcattacctacaaccagtcattaccaatttagtcattactttatgctgcgatacaaaaaagcctgttggtactttttcttgtacttgtggaatggtttattgccggactgggcctgatgaaacggatgatgacaaacttcgcattggtaaagtaaaggcattcggtcaactatgggaacaaaagctgaaagaatttgttgaagttgaaaagttgggtttgcgggagacagctagaagattaaatgttgatcccggaactatcaaacgctatgtatctctgctaaaactgaccacttattggcagactagaaaggaaaatggctctgtagacttacaggaagtagcagaaatacaccccaattctgcaactgaactcaaattgaaacacaggcaagattggatggctttacaagcacagaatcctgacgcttcaaagacaactctgagaaaactagcaccagccacctatatttggctatacagaaatgatagagaatggctcaatcaaaactcacctgcattacaagttgctgttccttccgttgtgagagttgattggcaggaacgagatcggcaaattctcgcccaggctcaagacgcagtgcgatcgctaattaacgccgatacgccagaaagaatttctattagtagggtaggaaaaactatcggcttactcgccttactcgaaaaacacctcgaccaaatgcctttaacaaaggcttacttagaatcagtagttgaaactgtagaagattttcagatgcgccggatcaagtgggcaatcaagttgcttgatgaatgtggagaagaaattatgcgctggaaggtagtcagagttgctgctttgcgggaggattgctctgaaagagtcaacgcatttctggaaaatgaacttgacaaagcgtacaagaaagagaggtgattccattcacctctccaagtgatgagataaatgctttgtgtgttatgctgcgtcggctaccagtaagccattcacagcgacgatgatgatttgcgctgccaaaggagtgtcacaacgtagcttatcgtcactattacagggttgtgctatccattgaccctccagattttggtagaaagtgccgagaagttgggaaccattccaaacacggtaaagttcaccaaatacctctgataccgaatcaatctcaatttccggtgctatttcctcacctagctgctcaatgtactcctcaaactctaattgagccaaggcttggttatcaaattcatcctgtacaatcatcatgagtcacctcgttaacggggttgaacaaaaggcgatggcgtaaaacgcccgcctaaaaggcgatcgcatttctcttgccggagggcgatcgcctttttgatttcagattttattttagctactatagtggctatatacaatatatgtatggaaaactttattcagtcacttccactgtgctaagaaaatcctttaaagaaatgcctaaagccaagcaaatagcctctaacaccttgatagagacactttcagactcgccgtactctagttttttcaggttttggtgagagcattccccacccttagcagaaatttcctctgccaatttctgccgggattttttaccacgcatacgctttaaaactgctgctctatctttattccagctaacggccacaacgtctgaaacataaatatacattttagtttctctattcctctcattgttcatataatagctacagcagtaaacaaaagttattaatttcggctactacagtttacaaattatgggttagaggtaagcgatcgcagttaatcaagttttcctgtcagtcaaaagagagtaactgtttttgctatgaataccacttgtgcatataatttcaaaggttctcccagaacaaatacattagccatctacagcatctagagccttgacacccgaagaactaaaacagcttgaagacaccctatggcagagtgcagataccctgcgggctaactctgacctcacatcgaatgaatattcgacccctgtaatgggtttaatatttctcaagtttgctgataataaatatcgtcagtatgaagcggagattttacaggcttatgaagcattaaaagggactcgtagagagaagaatatagaagaaatagctattgagaaatgtggtttttatttaccaaatcacgcgcgttatgactatttattaaacttaccagaagaggaagatatagctaaaaaaattaaagaggcgatggagtccatagaaaattataagccagaactcaaagacacattgcctaaagatgaatatttccgcctcactcgtacagacaaaggtattcctaaacaactattaaaaaatttttctaatattcctcaaaacgccactggggatatatttggacgaatttatgagtattttttaggtaattttgcactttcagaaggacaaggtggaggtgagttttttacaccccgttctgtggtgcggctaatggtagaaattattgaaccattaaaaggtagagtatttgatcctgcttgtggttctgggggaatgtttgtacagacaggacaatttattcaagaacagcaagaaaatcaaaatcagggtgtaattaatgatttatatgtttgtgggcaagaaaagagactggaaactgtaaaactagcaaaaatgaacattacagttaatgggttaaggggagaaatcaaacaagcaaatacgtattatgaagacttgtttaatagtcttggcgattttgattatgttctggctaacccaccctttaatgtggatgatgtaaatctgacacaggtggagaaagacccgcgatttaatacctatggtatcccccgcaataaaacaaaagttaaaaagcaggatgagggtaaacaaacagtacctaatgctaattatctgtggattaatttgtttgctacatccctaaaacctaaagctagagcagcgttagtaatggctaactcggcttctgatgcgcgtcacgcggaagcagatattcgccaaaagctgattgaagagaatttaatttatgggatgcttgctctgccctcaaatatgttttacacggtgacgctacctgcgacattgtggttttttgataagggcaagaccgatgataagattttgtttattgatgcccgaaatatttttacccaggttgacaggtcacatcgagaattttcagcagaacaaatcagtaatctggcggttattagttatttgcgtcagggtaagcgacatcggtttattaagttgattaatcgctattttcagcaaggaatggcgaagttaaaagaaaatcaatcacaggtaaagcctgtttctcagcaattaatggctgtgttgaatgatggtgcagatgatgtcaaagctagggaagttgctgttaatttcctcaatttgtgggatgaggtaacagatttagaaattcagtatcaaggttatttagataaatataattatcctggaataaatattgatacccaaaatgaggcacagcaaaaactaagtgccgcatttaaaccgttttttgataatttccatgttgagctaaaacagcttgataagactgtgcgcgctcgtgaagcagagctaaaggagaaagcaaaggaagcagaggcgaagaaacgggggcagattgataaacaaattaaggaactgaaaagcactttggaaacgctgcatgaggaggtaaaatcagcagaattattttttactcatattcgctggttacaggaacgttttcctaatgcaaaatatgaggatgtgacagggttgtgtaagttggcaactttggaggaagtgaaagaacaggattattcgttaaatcctgggcggtatgtgggggtagtgattgaggaagatgggaaaacggaagaggagtttattgaggagattttaggtttaaatgaagagttggaaaaattaaatcaagatgtgcatttgttagaggggattattgctcataatttacgtgagatagcgggggatttatgagtgattatttaaatcaatttcagaaattagatgaagtggggactattgcaagaggtaaatcaaaacatcgcccaagaaatgagccatcgttatatggtggaaaatatccatttattcaaactggagatgtaaaacatagcaatctttacataactgagtattcccaaacatataatgaaaaagggctatctcaaagtaaattatggcaagcaggtacattatgtataacaattgcagctaatatagctgatactgcgatattaaaatttcctgcgtgctttcctgatagtattatcggatttataccctacaaaaataaagctgatgttcgatttatcaaatattgtttagatacatataaaattcaaattcaggcaatttctcaaggcacaacacaagacaatttgagcgtagaaaaacttttatcgcttaaatttagaattccattaatttccattcagaaaaagattggggcgatactttcagcttatgatgatttgattgaaaaaaataatcgccgtattgccatattagaaaaaatggctgaggaaatctaccgtgagtggtttgtccgtctgcgctttccgggacatgaacaggttaagtttaataaaggcatcccggaggggtgggagagaaaaagattcgatgaattttgtctactccagcgtgggtatgatctaccagatgctaaagtaattcctggccaatatcctgttatcgcttcaacttcaattaaaacgtaccataatcaatttaaagttaaccctccagtaattacaactggtcgctcaggctctcttggcacagttttattcatcaatagtcaagcctggcctttaaacacgacattatttttaaaaaacttttatggtaattcaccatacttaatttactacacactaaaattcttaaaactggaaaattttaattctggtgctggtgtacccacactaaacagaaatcatttgggtggattgtatatgtctgttcctcctaaaaacttgcaaaataattttactgacaaaatagcaattatttttaaacagaaagagcttttagttaggttaaataataaacttgttgaaacacgcgatcgccttctcccccgcctcatctcaggtaaactttccgtcgaaaacttagacatccaattcccaccgagtatggctgacacaccaccataaaagtaatactcagtgtttatcctataaagtcagggcagatattgacaaatttaagcagcaatgggctaaaatttgtacattgatggactaataatatcaaaaacagcctatagccgtgtagtttgcctaaactatgcctaactttatctctgaagatgacatcgaacaagcggtactccgcaaattagagcagcagcacggttttaaattactcaactgctacaccccaaaccctgacgacctcaacgacagttcaaaccgcacagataaacgagatgtcatttttagcgatcgcctgaaagctgcggctatacgcctcaaccccaccttaccagaaacagccatagatgaagctttaaaaatcctcaccagtcaacgtcagtctatgtctcccatcgctgcaaatcgggaaatagacgggctgattcgtgacggtatccccatacaatatgaaaatgcccaaggtagaaccgaacccatcaaagtacgagtaatagattttaacgatttcaggacagatttcaataatgaatatttagccgtttcccaactgtggattaaaggagaaagaaattatcgccgtcctgacatcatcctctacatcaacggtttacccctggttttcattgaactgaaaaattccaacgtcaaattacaaacagcctttgacgataacctcaccaactacaaaaaagatattcctcaactcttccttgccaatgctttctgcattctttccaatgcactagaaaccaaaattggcagttttaccgcagactgggaaaacttctttaactggttacgggtagaagatgaaaaggaaaaaataaatcgtcagcagattcaggaatctggtactagcctagaacgagccatagatggtctttgtcaccctgccaaactcctagactacatcgagaattttattatttacagaagtggaactcagaaaattgtcgcccaaaatcaccaattcattggtgtgaatcgcgcaattgatgcctttatccatcgggaagaaaaacaggggaaattaggcgttttctggcacactcaaggctcaggaaagagcttttctatgattttttatgtccgaaagatttcgcgcaagctcaaaggcgattttacctttgtggtggtaacagaccgggacgatttagacgggcagatttaccgcaatttcttaaacaccgaaaccgtccaagagaaagaagctgctcaacccaaaaacagtaaacaactgcgggattttctttctttcaacaagcggatagtttttaccttgattcaaaaattccgctacaaaaagggcaagaagtatccaatattgagtgaacgtaacgatatcatcgtcattgttgatgaagcacaccgcacacagtataaatccttagcggaaaatatgcgggcaggtttacccaatgccaactacctcgcgtttacaggtacacctctgttaggtaaagaacgcaaaactaatgaatggtttggggattatgtcagcgaatacaacttttctcagtcaatagatgatggtgctactgtccccctgttttaccagaagcgagtaccagaggtactgattcaaaacgagaatttaagcgaggaattttaccaaatactcgaagatgaaaaccttgatgatacgcaacaagccaagctagaaaggaaatttgcccgtgaaattgaggtcattaaacgtgacgatcgcttagaaacagtagccaaagatatagtttaccactttccccgtcgtggttatctaggtaaaggtttggtagtttccgtagataaattcactgctgtgaagatgtacgacaaagtacagtatcactggaaagcagaaattaaaaatttagtgggacgaattaggaaatctaataatgaaatcgagaaagtgcgcctcaaaaagattctcgattttatgcgcgaaacagaaatggctgtagtgattagtttagaaggaagcgagagcgaagagaaaaaatttgctcaacaaggactcgatatcaaaccccaccgaaaaagaatgaatacccttgatgctcaaggtcatgatatcgagtatcaattcaaagattcagaaaatcccctgcgcttggtgtttgtctgtgccatgtggttgacaggttttgacctacctaccttgtctactctctacctagacaagcccatgaaagaccacacgctgatgcaaactatagcccgtgcaaatcgcgttagtagttatcagattaataatgtcactaaaaccaacggcgagattattgattactacaatgtcttccgtgatatgaaaaaagccctagcagcttacgctttgggcaatgatgaaaactccctagttccagaaaaatctgacctgtttaaattacttgatgatgcgatcgcgcaaggactaaacttttgtcatgaacgaggcattaatctagaaactatccttgccagtcaagataccttcaagaacttagagcagtttaaccagtttgccgacaccctactgcaaaaagatgaatggcgtaaagcctttgtggtttatgacaataccattacctcactctacgaagcctgtaagccagagatattaaaccaaccacgccccctggtgtcagtttttcaatacttacgaggagtcattgatagtattatcgagcgcaaagatattggtaacgtcagtttaaaaattgccgagttattagatgaaagtatagtcacagatgatcaaggagtgaccacacaagagtatagtgcgaaataccaaattatccagaaaggtgaagtttgggatttaagtaaaatcaactgtgagcagctaaaagccgaatttaaacacaaaccctataaaaatattgcaattaccgatttaaggagcttcatcgaagacaagctcaaccgcatgattcagcaaaatactactcgtacagactttgcccaaagactacaagcaatcattgacaaatataacgctggtggttcatcaactgagaactactacgaagccctggtaaactttgccgaaaacctgaaacaagaagcagaacggcatattcgggaaggcttgacagaagacgaactggaactatttgatttactcaagaaagacaaaatgactgcggatgaaacccagaaagtcaaattagctgccaagtcccttttacagcgactcacagcagagcagccaaaagttttagtgcaagattggtataaagatatgcagagtcaacgcagggtgaaatccgtagtggaagcagtcttagatcaaaatctgcctgaaagctatgatcgagcgttatttaagtttaaatgcgacacagtatttgatttgatctatgaccaggccagtaaaggtgcaaagtgggcagtctgagtaatggaaactatgtctatctatgaacgcgatcgcatgaagacgaaaatcagatttatgagatttgtacggctggcataatttatcactgctttcgacttttcaagtagctctacttaagcgataattagaacttgaagacacatgatgactataacaaaatgggccagcatttatacgctttacttgttggtatcgacaactatcctgaccccaatcatcgccttcaagggtgtgtgaacgacataacagtaatacaagagtatcttaacgaacgggtcgatcagcagcagtatgaactgcattcattgatacttaaaaatagtgaagccactcgtcaggcggttattaatggcttccgcaaccatctttgtcaagctcaacaaaatgatatagttctgttttactacagcggtcatggttctcaggagttagcaccgaaagaattttggtatttagaaccagaccatctggatgaaactttagtatgttatgacagtcgtactgagaacggctgggatttggctgataaagaattggcactactgattggggaagtcgcagaaaacaacccccatatcacaattattttagattgctgtcactctggttctggtactaaagatcccatgcagacagctaaggaacgtcgcttccccacagacaagcgatcacgtccattagatagctttatcttttccctcgacgatttagagagactttcagatacccacaaccccgaaaaacacccgactgggtggaaaatccccagaggtcgtcatgtacttttagcagcttgtcgagaccacgaaacggcaaaggaatactatggtggtggccaacaccggggtagtttctcctatttcttactggataccctttctaaaactaacggaaaactaacttatcgagatttatttgcacgcaccaacgctattgtccgcagtgaaattacagaccagtctcctcagttggaagtaaatgatcctgaagatgacaataaattttttctcgatcgcgtgatcgctgaacatgacccctattttatcgttcagaatcacagaacctacggatgggtaattgatggtggtgcagttcacggagttcaagcacctgaacatggagaaactactttactagcattgtttccctttgatgccaatattgatgatctacgtgacccatcgaaatctgtgggtatagcaaaagttactcaggtactaccaggtaagagcaaaatagatattgaaggtatacaaaacttagctgctgataacacttttaaagcagttgtgacgagtctacctctaccacctttaggagtgtactttgagggagatgaggatggtgtcaccatagcaagggaaaagctgagaacagtgggtaatggtaagtctccatcaccatatctccgcgaagagaaagaagctagtcaagctgagtttcgtctgttgtgtcacaacaaccaatatgtaatagccaaaccaacagatagccgacctcttgtagaacagattgatggttacacaccaaaaaatgcagaaatagcaattaaacgcctagaacatattgcccgttggacaacaattgctaaactttcaaatactgcggcaactcggattaaagctggtgatgtgaagatggaacttatctttgctgatgaggatttaccacaatcaaaccagatgcgtttgccatacaaatataataatggaacatggcaacgaccagaatttagacttaaactcaccaatacgacgaaaaagccacttttctgtgctttagttaacctgtcagatagtttctccattagtaccccgttttttgaagcaggtagcattagactggaagcagaacaggaagcttatgctttagatggagaagaattgcaactagaagtcccagacgagtattgggaacagggtatcacggagtacaaagacattattaaattgattgttagtaatgatgagtttgatccaagattgttaaatcaaagcaaacttgatgctcctcggcttaaagcaatagataggaattatgcaaatcaaagtagtcttgatcggttgatggatcgtactcaaaatcgagaaattagagcgaaaagtgacgctgttagatttgatgattggtacgctgaagaaatcacaattactacagtccgtcccctgcaatctacaccagtttctcagcaacaagaacaacaattaggcgcaggggtaagattacaaccacatcctaatttagtggcaaatgttagcctcacaactataccagaagtgagccgagatttgggtaacaacattgtacctcctatcttgagagaaaattccgaagttattcaaccgttccaatttactgccagtcgtgctactgaccctggattaagtgtattggaattaaacgatattcaagattacaaagttgttactcctaatgcacctctaaaattatttgttgatgcaccactagcagataatgagtatattcttcctatcggctatgacggagaatttttcttgccgttaggtcgtggtcataaaactttacatggtcaaacagaaattgagatagaacaattaccagccccagttagtcaaggagagaaaagcctacaaggttctattcgcatcttttttcaaaaaattatcaaccaaaaattaggaaaaaaatataactatcctctactcagagtagtagaagttaatgataatcaacaagttaattatctaccagatgaggtaactattaaccgagttaaggaagcaaaaagaattgctgtttatattcatggaattattggcgatactgaaagtttagtggcaaccatcaaacagcctatattacaaccagacagacaaacagtttctatcagcgaactttatgatctaattcttacctttgactatgaaaatctcaacacttccattgaaaataatgctcaatatttaaaaaagcgattgagagatgtaggtctgggagaaaatcacggcaaagaattacatattattgctcattcaatggggggattggtatctcgttggttcattgaaagagaaggaggaaataaagttgtccaacacttgattatgttgggtacaccgaatgcaggttctccttggccagttgtagaagattgggtaaaactcactttaggtattgcacttaatggtcttgctatcattgcttggactgcaactgtggtagctggactaatgggtgctttagaaagaaatattcgggtagcattagcccaaatgaacccagcttctgattttctcaattctctagcagccagtgatgaccctggtattccttactcaattattgctggtaatacttctatgattacatctgttgttccagcagcattagagcaacaaccagagaaaaattctagtgtgctagaacgactcaagcaaagtttatttaatagagttgtagctttaccttttggtggtgaaccgaatgatattgctgtgacggtggaaagtattaatagcattcctcaagggcgatcgcatcctccccatattcaagaagtaccttgcgatcacttgagttacttcctgaatcaacctgatgaagttgggttacaagctttgattgcagctattacgaatcaagaatattcttatggagagttacaaaaaccttataaagatcaagaattacagggactttatgaaggattacaagatttatgggaaaaacaagaaattcatacttgttctatttgctcgaattttatcacagttaatcaaaaatttgaaggtgctagtaatgttgagtctttcattgcgaaaaacagaaaaaaagtggaaaatataattgttgaaattaatagcacatattcggcaaaagaaataaaagataacttacatcaaattgcaattatagggttctacagaagtaacagtttccgttcgttacctgcaattatttcccaaaaagataattttttacaaatcgagcttgaagacatttttagctttttagatcctaaagaaaatcaacaaactgaaaatattccatctttttcaacagagataattttgtttgccgtttactatcagaaggagaataaacatggatactaagaaagtagaagaggtaaggaagacaggatgtactcttattgcttttaaagatggtaagtatgaaagagcagcatctgaagattttaaaacagaagctgacatagttattttaattcatggctttacgagtcatggagaatacatgggtcaggaattagtaccttatctttatagtgaaaattatcaaatcttcttgtttaattatgattcttacaaaggaatattatctgctgtaaggagtttacaagaattactggaaacgtatgataatctaactaatcaaaaaatacgctccaagaaaatattccttattgggcatagtatgggtggtcttgtagctcgtcaatttactatagatgatgcaaagaaggacaattcagataggtttataaaaggagtgatcatgttggctactcctaataatggtgtattacaaaataaattaagtaacgaaaattggagaaattttgttaagtatttaatatctgtttcagaagaaataggcggagtattcccacaagccagaacattggagtgcgtcgcggtaaaagaacttgctaaaattgatgagtataatattattgataaattgaattacgaatgggaaaagatgagttgcaatttgccaccaagtttatctatatctggtggacaaaattatcttgaattcactaataaattaattgaaggaataattaatcgtggaattcagcaattaatatctgattctaatacccgctgccctaatgatggaattgtgcttgaatgcagtgttgatatgaactcatgtattagctatccccaacaaaatcaacaatatatacacctgaataattatcatgaatattcaaataccaatcacaatacaattcatatccaacaaatagtatcaattaaaattgtagaatggttacaaacaatcagcaaaaaaaatacgagtttactaaataacgcataataataaaattgattgtagtgcttttgaatttttagagttaacataacaattatgtctaaacataaatttaaccttaattttgctattattatcggtattaataattatcaaagcgggatcaaagagctagaaacggcagttccagatgctattaaacttgctgaaattctacaaaagcagcatgatgctctaaaaccacaatatcaagcacaaaataaatatgaagtgcaattatttttaaatcaacgcgctggtcttaaacaactgaatcaattaattgcagattttaaacaaggtcaaataccattagacagaggtaaagttacagttacagaaaatgatcgcctccttttttactttgctgggcatggaattgctttagatgctctagaaaatcaagaaggcccagtgggttacttaattcctcaagatgctatatcaggggatagcagtacctacttgccaatgcaggaattgcatgatgctttaaactcacttccttgtaggcatatgttagcaattcttgattgctgctttgcaggagtatttcgttgggcaagtctaaaaagagaaattgtgcctaaagtaaaagtatataaggaacgctacgaccgattcattagcgatcgcgcttggcaagtaattacttctgctgctgatgaccaaacagctttagattctttaggttcacggggtatggtcacagaggggaacgaagttcattctccctttgccaaagctttgtttgatggtttatttgggaagggtgcagacctaaataaagatggtatcatcaccgcaactgagctttacttgtatttaagagatcaagtcgaaatccttacagaaaataactacaagcggcaaacccctggtttgtgtccactgaaaaaacacgataaaggggaatttatctttttgtcgtctgaatttgaccgaaataatttggaggatgcaccgccactaaatttagaaaataatccctatcgaggattagaatcctatgatgaaaaacacagtcatttgttctttggtagagaagaacagattaaaaagctttaccagaaagtagttgctaacaaccagaagttaacattagttttgggtgcttcaggtactggcaaatccagtttggtgaaagccggacttattcctaaactcaggaaagataaaacttggtttatcctacccccttttcgaccaggggagtctcctttcaaatcattgaataacgcactagaatcggttaagcagcctttgattgctgcttcctcaacgacgacaccttctcgtttgcttaccccagccgaagaaagccttgcaaattggtttaataatcatcctcaagcaaagctactggtggttattgatcagtttgaagaactgataactctgtgtaaaagtgacaaagagcgagagcaatttgagcaactgattaaaaatgcaattaccaaatatgccgacaaaatccatgtagttatcaccttgcggcttgactttgaagcacagcttcaaaactctgtacttgaaaagttctggaatgatgagactcggtttgttgtcccaccaatgagccagaatgaatttcgggaagtgattgagaaacctgcactagaaaaagttatatattttgaccctcccagcttggtagatgacttgattaacgaagttgtacaaatgccgggtgctttacctttgctttctttcaccctcagtgaattgtacttgaaatatttgcaggagcggagagataaccgggctttaacgaaagaagattacgaacagttaggaagagtagttggttctttaaccaaacgagctaatcaggaatatgatcatttaattcaagaaaaccccgcttatgaaaacactgtacaacaggtaatgttacgaatgatttcattgcagggtagggagtcagcacgacggcaagttcctaaatcggaactggtgtatgccaatgaggaagaaaataaacgggttcaaagaattattaaatgcttttctgaagctcgcttaatcgttgagggttctaattctcaaggtgaaccttatgtggaaccagcacatgatgctttagtacaggggtgggttaagttacaggagtggattaaacaagagcaacagaatttacctttacaacgacgattgacattagctgtccttgagtggaaaactaagaaacaaagccaagataggcaagaagcaaagttcctttggaatgccgatccttacttagatgttttaaacaaaatactcaattccagtgacaattggcttaaccaagtagaaactgagtttgttaagtgtagcaccaacctcagagaaaagaatcgtcgcattagaatgggcttggttgctaccgcattctcttttatctccatattttcttttatacaatggaaacaaaatcaagaagctcagagtatcaacttgagtgtatcttcaaaaactttatttgctgctgatcaacaaatagaagcgttagtagaagccataaaatctgggaaaaagctcaaatctgtggttataccagaagcaagtaccactcaccaggttgtaacaactctatatcaattagtttacagttcgatagagtataatcgtttgcaagaagaaaaaggattttatcgtgctagttttagccctgatggcaagatgctggtttcagtaagtcaggacaatattatccaattgtggagcttagatggtaagaaacttaagaaaaattttaccagtaatctttcaagtgtagatagcatcaactttagtcccaacggcaaaatgctggctctggcaggtaataatgataattttatcgaattttggaatctggatggagaagataaacccaggacgcttaaaggaaaactttcaaaaacaaatagtgttagctttagccccgatggtacaatgctggctttaggaagtaacgacggcatcatccaattatggaatttagatagtaatgaagttaaaacttttactactaatcttaaaataaatagtgttagctttagccccgatggtacaatgctggctttaggaagtaacgacggcatcattcaattgtgggattttcgtagcaatgaaatcaaaaattttaatagtaagtataaaataaaaaatgttagctttagccccgatggtacaatgctggcttcagtaagtacaggaatcattcaattgtggagcattcaaacaggcgaaaaaatcggtgcgggaagggaactcgtaggggatgtggattgggttagtcgtgttagcttcagtcaagatggcaaaatgctggcttcaggtggctataacggcattattaaaatctgggacactgaaacaggtaaagaactcttaagcatccaagggagtcgaggtaggactggtgcgtcaattatggatatcacttttagtcctaacggcaagatgttagcttcagtagatgatgacaagtttatcaagctctggaatctgaatggtgagaataaatccaagatgctcaagggtcagctttcaaagataaggaatattagctttagccccaacggcaagatgttggctttggctggtgatgataagtttatcgaactctggaatctagatggacaggataaacccagaaaacttcaactacaagtttcaaagataaggagtattggctttagccctaacggcaagatgctggctttagctggcgatgataagtttatcgaactccggaatttagatgaacagtatgaacagtatcaacctggaaaacttctaggacataagtcacgtgttaatagtattagttttagccctgatggcaagatgctagcttcagcgagcgatgaccagacaataaaactatggagtctagatagaaacaaagaacccacaactttaaaaggtcatagtaatttggtcaattttgttagttttagtcctgatggcaacttcctagcttcatcaagtaaagaccacactatcaaactctggagattgaaagacaataaggaagtctggagtaagaaaattgggtctggttctggtggccaaacctggtgggttggttttagccctgatagcacaatattggcttcggtgggttcttacaattttatccagctctggaatctagacggtaacgaattcagaaggcttagaggtaatctttcaaatatcaatagtgtagccttcagtcccgacagcaagatgctagcctcagtagaaacgacagaagattcagtaggtaaagcagaggaaagcgtcatcaaattcttaaacttagaggggcttgaattcaataggctcaaaagccaatttgtagatattaataccatcagtttcagtcctgacggcaaacttttagctgtaggagttgaagatggcactgttatattacacaacttagatttagataatttattaagacgcggatgtcattggatacgtgattatttgcgaaataaccctaatgttagtgaggtcgatcgccatctctgtgacgatgtaccaccaattactacaaatcctgagaattaggtgagttaaaagcgaactgcaaccaccatatgtccaaatccaagtacgaattttaccgcgaccttgcagtaatcattgggattaataactactcaaatggtattccagagttagaaacgcctgtcgctgacgctgaaaaattcgccaaaattctccaagataattatcaatatgaggcgcagatattactaaacgaaaaagcgacattgaaagagttaaattctctactggaggacttcaagcaaaaaaaattacgcctaccagataaaactgtacagatagaagaaaatgaccgcttaatactttactttgctggacacggaatcgtcctcagagatgggctagaaaatacggataatctggaaggatatcttgttccccaagatgctagaggagatatcttgttgcaaaacaatattcttctacctatgcaagacttgcatgatgcactagcacaattaccttgccgtcatttgctaattatacttgactgctgttttgctggggcatttcggtcgagtttgtatagagaaattgtacctgcacgaaaagtttataaacaacggtacgatcgctttattagagatccagcatggcaagcgatcgcatctgcggctcatgaccaaaaagctattgaccatctaggttgttttggacagagaggaagcaaaggcaaacactctccttttgcaaaagctttgtttgacggtttgctgggtgctgctgacacaactctaagagaaggtgatggtattatcacagcaacagaactttactgttatttacgcgatcgcgttgaggaactaactgataaatatgacaaacgccaaactccaggtttatttcctctgaaaaagcatgataaaggcgagtatatcttcttgcttcccaacttcgaccaaaacaatttagaagatgcaccagcactcaagcaagaaaacaatccttatcggggtttaaagagctatgaagaagaacattctctcctgttttttggtagagatgaacttgtcaaagaactatctacccatgtttctcaaaccaataacccactcaccgtagttttgggtatttctggttctggaaagtcgagcttagtgaaagcaggactcattccttatttacgaaaacatcataaccaagaatggtacatcataccaactattcgcccaggagataattctttcacttctctggcagaggcacttgtagaaaagtctgaaacagatggaaattacttaaacaaaattaaatctctaaatcagacattaaaacaaataccacaggattttattaagataatacaagaattactactaatacctgaaaaagtaaaaattttattagttatagaccaatttgaagaactaatcacagtttgtaatccacaagaaaaagaacaatttttaaatttccttagcagtattttagaagctcatcctcagcaaatttccattgtattgactctgcgttctgactttgaacctcggtttttagattctgctctgaaagcatattggacacaatctcgttttcctatacgagctatgagatccgatgaattacggcaggcaattgaacgacctgctaacgaaaaaatgctcgattttgacccaccaaatttagttgatcagttgattgacgaagtggggcaaatgccaggagcattgtcgctattatcttttacattgagtgaactatatattaaagccataaatcgagaaagcagaactttaagtaagactgattacgatacattaggcggagttgcaggttcattaacacaaagagcaactcaggaataccaaacattagtgaaacttgatccagcttatgaacataccgtccgccaactcatgctgagaatggtagctattgaaggtaatgaattagcacgacgacgagtaccaatgtcagaactggagtatgagagtgtagaaaaaaatcaaagggtgtcgcgggcgattgctcagttttcggcggcacgtctaattgttgaaggtaaagaaactggtgacgaaccttatgttgaaccagcacacgatgccttagtaagcgggtggaacaaactacaggaatggaaaaatgatgagcaaggaagtttagttttgcgtcaacgcttaacaccatctgctaacgattggctaaaagatgggcgcaaatctgattatctatggattcgtgatcctcgcctcgctatcctggaaaaagtcctggaattgccatctgaagaaaactggctcaatcagctagaaacagaatttgtgaacaccagtattcagaaaagaaaggatgaacttgaagaaactaaagaacaactccgcatttctgaagagcgtcgtatcgaagcagaaaagcaaactgcgatcgcgcttgctagaaattctgaagccttactatcttctaatcaacaattagaagcactgattgcagcagtaaaggcaggaagaaaattacaaaatctactagcagaagaagctatttcttcacaaagtttgttattcaagcaaactgtgttattcaagcaaactgtgattgccctagaacgagcagtttatggagtgagagagcgtaaccgtttggaaggacatagcgggccagttcaaagcgtctgttttagtcctgatggcaagatgattgccactgctagttgggataatacagttaagctatggagtagggaaggtaaagaactaaaaactctgaaaggacattatgatggtgttttaagtgtctgtttcagtcctgatggtcaaactgttgcctcaactggttgggataacacgatcagaatatggaatctggagggacaggagcttacacagcaccctctcggatttggccatacagatcaggtatggtgtgtttgttttagtcctgacggagaaatgatcgcctctgccagtcatgacaggagagttaaactgtggaataaagatggacagtttctcagaaacctttgtgaatcaagtaccaccgagcatgaactaaccgtcagtagtgtttgctttagtcctgatggaaaaataattgctactgcaagttatgataacacagttaaactttggaatcttgacggtactttaattaaaagttttacacatagcggggcagtttatgatatcagtttcagccccgatggtaacactattgtttctgctagtgcagacaagacagtaaaaatttggagtttaaacggcaaaaaacaaactcttaaaggacatgaaggtgaatttacgagtgttaggtttagccctgatggaaaaataattgcctctggtagtaaagataattctatcaagctttggagtaaagacggtcaggaacttacaactctcagagggcatactgcggaaattactagcattaattttagccctgatggtaatatcattgcttctgctagtgaagattcaactgtcaagatttggaacaaagacggtcaacaaatcactatccttccagaatatggtgaaggggttactcaagtctgttttagtccagacggcaaaacaattgttggtaatgcgaaaaatacactcaaactctggaatcttgatactcaagagagcaaaatgttttctgatattactggtgcaaacaatggtatcagtttcagttttagcccagatggtaacgcgatcgcggtctgtggaggttttgacaaaatggttaaactttgcaatcttgaaggccaagaaattaagaactctaacgattattatcacgcaataagggctatccggttcagccctgatggtaaaactattgcttttaataataataaaaagattgacctttggaattgggagaatcaagagataaaaaccctgtcagggcatacaggcaaggtttgtagtctatgtttcagcccagacagtaagataattgtttctggtagtgatggttctgataagacaattaaactgtggaacctaagcagtggtgaagttatcatccttgaaggtcatagtaatagtgtaaatcaagtctgtttcagtccagatggcgaaataattgcttctgccagttctgataacacagtaaaactttggaatcttaatggacaagaactaaaaactctccgaggtcatagctatcaggtatttgatgtaacttttagccttgatgggaaaatgattgcttctgctgattgggatggcacaatcaaactttggaatcgtgatggtcaactgctcacaactttagaaaaacgaagtgaaaaactatccagtattagcttcagcccggatgttcaaatcctggctgctgccagtaataaaggagtaattttatggaacttcaatttagatgacttagtagcacgtggttgtaatttgttacatgaatacttaatgaataacactaatgttcaagaagagcaaaatctctgcaatgacagatgaataagcagtaagcaatatacgcaaaatagttcacagaaaataactgacattctcaaaactaaattagttattaacccaagttgctagttatgccgaagagcgatcgcagtgggttccattgccgcttcagaatggatagttaacatatactctcaaggcgactgtatttactgcttccctgcgtggcgtagccattcctgttacttaatgtttccggttttagtcgatactattaccatagcagcaatagagggattactgattaagcttgctctggagagcgatcgcaagttattttggaacaaatagatgccaaatccttacagggcttgctttcggcaaattatatgacttttttatttatcaatatgaggcatttctgaaaaattgtacaactttattttttgatatcaaataaaattgaacgtaaatacaaggcttataaactttcaaattatataagttttttatgtttttacaataatttggtagaataaccagataatttctgaatgtgatgataatattatcacaatcaggaatatttttttgggcgatcgcctacggtgctgatgcgtccacctcatcacttgtctataaataaaaaacaagcaggacatatactatgagccaaaaaaccattgattccaaaaatctgaccatagaaaaaatttttgaagatttttatattattcctagttatcaaagagaatatatttggaaagaaaaacaagtattagagttattaaatgatgtatatagtgaatttcctggctctttaaatatagaatctgatgaatattttctaggcagtattgtaatttgtcctagttctgaagatacagacaatgtgtatcaggtaattgatgggcaacaaagattaacaacttcttatatcttcttttgtgctgtcaaaaattatataaataagttgccagaacatcaaaagtttgatgcgattaagaaattaatttattcagagtatacaaatgcacaaggtcaagaccaatcacaatacaggataaatcctcaatatccagacagtaaaaatattttggaagaatttgctaaagatgaaatcaatatagaagagatagaaaataaaaaaaataataaaactgaatctacaattaatttgatttcagcttaccaaacaatagaagattttattaaggataagttcaaagatgatgaaaaccagatcaaaaggttttatgcacatttcatcaaaaaagtaaagttagttagagttgaaactacagatattaaccacgccttaagagtttttgaaaccatcaataatagaggcgttggcttggattctatggatttacttaaaaatcttttgtttaaacaagttgaaaaagaagatttttcaaaaattagtaattcttggaaagaaataaaaaatgaattagattcagtaaaggaaaagcctatggattttctacgatactttattctaactcaatatgatgttgaaagagataatgtccaaactaaagaataccaatggttactggaaaataatactacttgtaaatatgaagaaaaaccatttgagtttgtcgaggacttactgaaggctgcaaaggcttataaatcattactagaaagaaaaaatccttctgataattcagataacccgcatttagctaatattaagtatttaagccccaatcttaaacaacatctatatatactgttagcagcaagggatatgtcaatggaaatattttctcaattgtgtctacatattgaaaactttatttttgtttatagttttcctaataattcaatcagtgaattagaaaaaatatttattaattggggtcaagagattcgtaaaatagtacaaaatacagacaaagaagagcaattatctaaattaaaagaatttttatataacaatctaggtgtacgtaaagaaaatctcaagaaatcttttgagaaaaagtttaaggaagcaagtcaattagactttaattcaagaggaacaaataaaaagccaacatcaaatgctaataaaaaaacaaagtatatattagcaaaaatttctcagtatgtacaacttatagcttatagcgacaatgatgagtgtaaaaattttacaacttttactcagagttcaatagaaattgagcatattttaccacaagatttaaattctgaacctgttaaagcttttgataaaccagatgaagttgaatcatatatatacaaattaggcaatttagctttgctagaacaaactctcaatgcttctattcaaaatagtccatttgaagttaagaaagaagcttatcaacaatcgaattttcttttaacacaatgtatatctgaaaagcccactattggaactaatactgcaattaatagggctgtggagaaattaataagttttaatgaatggaattcacaatcaattgataaaagacaagaaatgctcacagaattagcattgaaaatatggtacgaggaagtatctttcgaagaagtaccttttgaggaaagtctggatgctagggaaatacaggaactattgcggtttggtggtgaaagagctatttaatttgatagcatctgatatcctttctacacctaaagtgccagttgcgtaagtcctgaacatatatagatagaaagtgagataagaacaggtaagagtaagctattcaaacacaacgccaattttcgatttgtaagttaggtattctttgaaattctcgaacattagatgtcaccacaatatgattattgatgactgcggtagctgcgatgagtacatcatacgcaccaataggtgaaccagctagtttgagaaagcttctgatttcggctgcttgttctgctgctttcgagtcaaaaggtaaaattgtgattgagtttaataatgtttcaaaaattgattggattttaacagcgcgttgtgggttaatagctaatccatatttcacctccataactgttagagatgacacaaaaatctccgcaggggaaattaattttattcgcttgagggtgttttcttctcctttgacaaaatcactaatgacgcaagtatccaatagataacccatacataattaaagttctatttcgttaggaggtatcagttcatctcgatatgattcaaagataatactttcgtttacaccttgatattgtattagggtttctggccattggctgggtttagtttctaggaatgtcacgaaaacaatgctttcagtaataccttgcggtgtttcagctagttcaattttgccgtttttataaattccttgaatggtttttaacatagagcttacctcctacattaagtttaacctatttattctccttcttcagtcatgctgggtgggaatcgaatgtctgagtcttcaatataaattttacctgagatgagacgatttatcaggcgatcgcgtgtttgtttaaggtgtaattttactaattggatataatcaatttgattgttcaatttacttgcaaatttacaaaagtttactgtgt

SEQ ID NO: 66 - DNA, 37 bp, Artificial Sequence
  Synthetic
actgctcaacgtctaacgacatctaatgttaaagcac

SEQ ID NO: 67 - DNA, 37 bp, Artificial Sequence; identical to SEQ ID NO: 66
  Synthetic

SEQ ID NO: 68 - DNA, 37 bp, Artificial Sequence; identical to SEQ ID NO: 66
  Synthetic

SEQ ID NO: 69 - DNA, 37 bp, Artificial Sequence; identical to SEQ ID NO: 66
  Synthetic

SEQ ID NO: 70 - DNA, 60 bp, Artificial Sequence
  Synthetic
atgccgtcgttacttcctaaagccagcattgtaccatcggggctaaagctaacactattt

SEQ ID NO: 71 - DNA, 60 bp, Artificial Sequence; identical to SEQ ID NO: 70
  Synthetic

SEQ ID NO: 72 - DNA, 60 bp, Artificial Sequence; identical to SEQ ID NO: 70
  Synthetic

SEQ ID NO: 73 - DNA, 60 bp, Artificial Sequence; identical to SEQ ID NO: 70
  Synthetic

SEQ ID NO: 74 - DNA, 39833 bp, Nostoc sp. NIES-2111
  misc_feature (34457)..(36961): n is a, c, g, or t
ttataaaaatacctagtggccaaaggagcaaacctgctccttttttctttggtaaagtttgttgatgattaacggttgactgtggactatggactattgactatgaactgttgactatactggaatttaattctcttgaagaaagggtggtgtgagcaatgtgtggcatcgttgggtatattggcactcaagcagcgacagaaatcttattatctgggctggaaaagttagagtatcggggttatgactcagcaggtgtagctacgctttgggaaggtgaggttcactgtgtccgagccaagggcaaactgcataacctccgttctaaactagagcaactagtaacctcctctcaaatcggaattggtcatacacgctgggcgactcatggtaaaccagaggagcataacgcccatcctcatttagatacagcaaggcgcatagctgttgtccaaaatgggattattgaaaattaccgcgagttacgggaagaactgaagcagaaaggatatgagtttcgttctgaaaccgatacagaagttattcctcacctcatagccgaattgtcaaaaaatgcctcctcttttctagaagcagtacgccaagctgttagccatctgcatggagcatttgcgatcgccgtcatttcggctgattaccccgacgaattgattgttgtccgccagcaagcgccgttagtgataggatttggtcaaggtgagtttttctgtgcttcagatacgccagccatagtttcccatacccgcgccgtcttacccctggagaatggcgaaatcgcccgtcttacacccttgggagtagagatttataactttgctggcgacaggctcaaaaaacaaccccggatgctcaacttgagtcccacgatggtagaaaagcagggattcaagcactttatgctcaaggagatttatgagcaaccaggagttgttagagcaagtttagacgcatactttaatacagaggttaattctggtaaatcattccaatcaccagttaagttaggtttacccgaacagttctacgcagacttagaacaaattcatattgttgcttgcggtacaagttggcacgccgctttagtagggaagtatttacttgaacaattagcaggaatctctactcaggtacattatgcttctgagtatcgttatgcaccatcaccgctaacagcgaatacattaattattggcgttacgcagtctggggaaacagctgataccttagcagccttagcaatggaaaaagaacgccgtcaaggtaaagaagcaaaatatcaagcgcaattcttgggtattaccaatcgcccagagagtagccttggtcatcttgtaccccatattatcaacaccttggcaggaattgaaatcggtgtggcagcaactaaaacctttgtcgcccaactgatggcgttttatgccctagccttagatttagcctatcatcgtcaaactgtagcgccggataaattagcagatattattcaaggtttgcgccagatacccaaggaaatagaagaaaccttagaacgccaagaaaaattaacggaacatttagctcatgaatttgcagagactcaagacttcatctttttgggtagagggattaatttccccattgcattggaaggggctttgaaattaaaagaaattagctatattcacgctgaaggttatccggctggggaaatgaaacatggccccattgctttattagatgcaaaagtgccagtagtggcgatcgctttccctggtactgtgtatgagaaagtcatttccaactctcaggaagccaaagccagagattcccgcttaattggcgtaacgccagtcagcgatggcgaagccggggaaatctttaatgatttacttcccgtttcccctgtggatgaattactttcacccatcctcacagtagtacctttgcaactattggcttaccacatcgccgcccgtcgcggtttggatgtcgatcagccaaggaatcttgctaagagcgttactgttgagtagtgttgaatcgagaaatattgaggttgtggaaacataataaagttgtacaataaataataaagttgtacaatgcctaataaagttatacaatttctcctatgggtgagtataattccatctgtaggagattgtctttatggctagaaccaagcaagagtggacgcaagctaagtttgagcgctacatgaaggaaggtcgaggtcagggtagtggaaaaaattatcaaccgtggataaaaattcaagattttccgtcgaagggtagggtatctagaccacccggttggaaaacaaaccgagagcatcatctgttctccgatcatgagaaacggttattttatttgtttgagtggtcggacagcattattgatataagggaacaattccctttaattgacttagacttagcgatgaacattgctaccgaaatggcgattgagtatcccaaagatgttaataataatactccctatgttttaactacagactttatgttatctgtgaagcaaggaaagcagatagttgaaaaagctcgcactttcaaattagttaaggatttaggaattaaatctgttgctgaaaaatttgagttagaaaagcgttattacacagcaaaagggattgattggggtattattacggaaaaagaaattcctacgcttttagtaaaaaatgttgagtggattcattctagttataagttagaagcagccgcagaaatcaatgttgatgagttgaataacatcgcaaatattttaaagtcaaaattgcaagaaagcgattctactattaataaaattacaaattgcttagataaagagatgaatctagaaataggtacgtcactctacatttttaagcatctccttgccaataagaaaataatcatggatatgcaaacaaatgcaatatctagttctatttcagcgcgagaaattcaaaaaatcattttctagaaatttatattggttgttcccatgaataataatttgtttgttaatgacttaattgagtggattgacgaatcaggaaacaactttgtagagagaattatttggattgatgaagcctatatcattgcctttacgatagatataaatatcaaaactggatttccagtgtctaaaagagtatcagatatccaagaagcaatttatgaaggtcgtgcattgaagttgaggacagacccttgggcaagaatcgttagagatgaagacttatctgctaaagaaaaagaaattagagataagtattgggatattatttcatctatagtaattcaagaaccatctatatactatcgagataaacgaggctcgctcattaaacaagttatagaaaaatataataataataagagcgaaggaaaattaattgaaagaagcgtttatcagattattaggagatactggcaaagaggtaaagataaaaactctctcttacctgattatgcaaattccggtggcaaaggcaaagtaagagcatctagagaaaaaaagagaggaagaccaagaaaatatgctcatgagccagacatcggtgtaggtataaatataaccgatgaagataagaaaatatttagattagcagttactaagttttatcacaatcaaaaagaaaactttttgacaactgcttatgatttaatgattaaaaactattattcagaggaaactatttacgatgagaatggcgttaaaaaacatatattaattcccccagataaacgaccaacattaacccagtttaaatattggtatgacgttgaacaaggagacattagaaaaaaaataatttctcgtaggggagctaaaaaatatgctctggaacatagagcaattacaggaaactcaacaatggaaactattgggcctggttctagataccaaatagatgcgacgatcgcagacgtgtacctagtttctgcgtacaatcgcaactggattgttggtagacctgttatctatgtagtcatcgatgtttttagccggatgataacaggagtttatgttgggtttgaaggcccttcttggataggagcaatgatggctttagcaaatgcagccagtgataaagtagcattttgcaaagaatacggtatcgagattacagaagatgaatgggcttgtcatcatattcctgatgcaattttaggcgatcgcggcgagttagcagggatggcagtagaaacactaattcccaatcttggtattcgtatcgaaaatgctgcaccctaccgagcagattggaagggtttagtagaaagacattttggcattatccacggtcatatcaaaccatttgtgccaggttacatagacaaggattttagggaaagaggagcaagagattatcgacttgatagcaagcttgatatagagcagtttactgaaattattatcaagatcatcctttaccacaacaatgaacattacttagataactatgaaagagatgaggcaatgatagctgatgatgttccccccatacctagagaattatggcagtggggaattgcaaatcgttcaggtagacttaggactattcctgaagatattgttaagctgaacttaatgccaacagaaaacgcaacgattaccgaacgtggcattaaatttaaaggaatgtactacacctgtcaaaaagcaaaaaacgaattttggtttgagaaagctagaagtaattctctttctaggttagagaaaaaattagaaatttcatatgacattagaaaacctgattatatatatcttcgctctcctgatggtagaaactttgagaaatgttttcttttagaatcagaatcaaaatattttaataaaaatttacatgaaattgaatatttatttgcttttgaagagttacaaaaacaaaaaaataaaggtttaaaacaacaacagaaagtagacttaatcgcagaaattgaaagtgtcgtgaataaagcaaaaaaaatgaccgaggaaatgaaagatgataaactgagcaatagaaaaagaaccgcaggtattcgagataatagaagtgctgaaaaatctaaacgtcgaaaaattgaaggctttgagctagaaaagagtgatcatgaaaatactagtaatactgtggatattgaaagtcaaaaagtagagcagccacaatcattgcaacctaactatcttgatattcttcgtcagaagcgagaggaacgaaagcgtgaacaagataaatgattggaaatgggttgaaattccaaatggtaaatctgctgctgtagctaaatataatgaccataaattaccagagtacaataataaccctttaattcaagcattacctccaattctttctggagaagaatttattgaaaaagcaataatactccccagttacgcacctgaagagagggaaatggatgcacaccgtcgatttcactgcgttgaacgattatctagatactttcaacctttaaataaaacggttaatttacagcaggttatgtgtgcattattaatgcagggctacatagcacgtaatatttccaaacctgaatatgcacgccgatcccgacaaatttatgaagcgataaaagcgggagatggtagaaatttagatgtttatgtaaatgttcccacatccacatccgcatctggtttaacattaattggcccatcaggcatgggtaaatctactaattttattaatattcttaatttatatcctcaagtaattctacatccagaatacagtacttaccaaattgtttggttaaaagtcgattgtcctcatgctggttctctcaaaggcttatgtacagatatatttcttggtatagataggttactgggaaccaactattttaaaaaatttggtgcatcacgaaattctgaagattatatgctggctcaagttgcccaaatggctcatactcaccatttgggagtcttagtgattgatgagatgcaaaatttagtaacagcaagaagaagttgctctgatatgctgaatttcttagtaaaaatggataacatcattggagttcctgttatccgtattggtaccaatgaagcttttccaattcttcaaggtaattttaggaatgctagaaggggaactggtgaaggaagcgttatttgggacaggatgagaaatgatgatgaatggtacttttttatggagaccatgtgggagtatcaatggacaaaaatatctactgatttttctgatgagtttaatgatgttttttatgatgaaagtcaaggaattattgatattgccattaaactttataaaatggttcaatggagagctatttcactgggtggaaaagaggcaataaacgccaatttaattaaacaatctgttaaagacggattatttttagtaaaaccaatgctaaatgctatccgttctggagacaaggaatggatgattaagtataaagatatagctcctattgatactacagaatattacaataattgtttatctaatttagaaactaaagatttaaaagaaattagtagattagcgaaaaaacagcaagctgagaatggacatttatcagcgacacaacgtcatataattcttaaacttttagaattagatgtagaaccgcaattagccaaagaatgtgcagtacaagtaattaattctggcgaagaagatgctgatattcctattttggtaaaacaagcttatgctctagctttgggtgggcaagttcaggaggctactgtaaagaaagtgagaacgcacacactaaaagcaaagccaaaatatcaaataaacgatattcgacaaattgcggaaaaagcaaagaaaaatcaaactagtgcatatgaaggtttgaaatcgacaggaattatcaaagatccagtccaggattttatcactatcacctaattatatgcttagtttctttccaacactatatcctgatgaactgttatatagtgctttggctagatatcatatacggagtggcaataaaacctttaagcaaactgatttagaattatttggttacagttctcagcaactatgtaaagttactttaaccaataatttgaattatttagttaaaaacttgtctttattttctcagttaacagttagtgatttactacaaaatcataccctataccctttttatgcaagtttcttaataccccagcaagcttggcaattacaagactctatgaggaaaaagctaggtggctctattttagaggttgcaaaagtagctaatcactctgctgacaactcaacaaagtttttaaagttttgtcctatatgtctagaacaagatacacaggaatatggtgaaccatactggcatcgaattcaccaaatacctggtatattagtttgtgcaacccatagagttgttttgcatgatagctctgtagctgttgattctaagggtatccattattatgcagcgagtccagagaattgtctcaccaatcaaaatcaagtgaaatgcgctgataacactgtagaaaagttattaatcttagctcatgatatttggaatataagtactattaatattccttttaaaggtttaacttggctccgcaatcaatatcagtcctatttaattaatcaaaagtttatgaccctcctacctggggagaaatttagatttaacgaaaaatctttttcaaattctatctgtgatttttatggacaagagtttctagaaataattaatccaaatttaatcaggaatccagacaaatatttttctggttatttactagcttgtgatttgaatccagtaatcgaccggattacacatatattgataataaaatttttagctaattctgtagaaatattttttacataacatacttgttagagtacttaatatataaaaatattttttagcaaaaaatatattaatttacttgaaattgcataccgattaaagatgtagcaatagagtatggtagagaaacaacataaattaagctcagaagtagcgatttttcagagaaatgaaataaatgggtgataattttcacgtatcaactcagagccatgctacagaggttttaaatcctcagttaatctacgtggacttaagctttcgtgtaattggtcagactttacccatagaccacggctatgctttgtatgctgcactgactcacttacaggacaaacttcattctctagatgacctcagcattcaaactattcctggcattcctgatggcaacgattcacttcatcttaataaccactctaggcttcggattcggctaccagtagataaaatacctttagtttatccgtttgcaggtaaatcactaacaattggcaggcataagattcatttagaaattccccaaatgtatcttcttcaagcggtggagaaattgcgatcgcgtattgtggttatcaaaggctacgaagaaccaaagggttttttagcagcagcgcagcgtcaactggaaaaactgggtgtccagggaatagccagtatacctaccaaagctgatggcaaaccagaacgcagaagtatcaaaattaagaaatttacagttgtgggttttggattggaggtaacgaatttaaatgatgaagattcaataaccttgcaaaaatacggtgttggcgggaagcaaaaaatgggttgtggagtttttgtaccagttaacgaaaaacgatagcagttgtttaaaggcagatgataaatacaacagctaactccaaaattcagttaaatctgagcgactctagcactactttgttacatcgcgcagggatagcaggattttggatgacgctaaaacagttggaagaaatatatccgaatcctgctcaacgtactggtaatttaacctgggtactaacaccccatagcatcagtcttaactgggaagggcaggatttcgcagctttagactggttgctaaagcaatcttttcaaattagtgatgagggtttaattttcctgacaggattaaatccaaaagcaattcatattgaaagacaaataataatccatcaaggtattacaggaacttttttacagcacaataaatttttcaaattttcagacaaaaaattaaagtccataattgttgacggcagacaatttgtgactgagtacaaaagtgctatgtcttatgctcatcagcactttgctaaaaagttgtgtgatgaatctggccaattgttacaagagcctattggcatagtaggttggctgtatccaggagcagtagttcgtcattctgcttttccaaaacaaaccaaatttgaagagaagccagagtatgcactagcgttattgtttgtgcctgtagcgtgccactattttattttgcgatcgcacaccaaaccccaatctacaaactatattctggttgtccccgaagtaaataatttagagttttacgcccagcattgctggaatctgagtaatttgagttacagagattttcatgtatctagcttaagagacgctggattaaaattcctgactaatgaagcagcaatacagataaaacaaaattgtgttaagcgatgtcaggttttttcttttgggacgctaatttggtcagaacagcaaaaagttcgtacagaaacagcaacattagagctaacggaaaaagtgagcttctactataagtttagtcggagttgtttctccaattaccgaatcgttgactatgaaaatcaacattttatccttacaagtctagttcaaggaataattgctaataatttaggtttagggttgccttggtggaatgattttggcaacatacttacatctagtaatttgtttaatcaaatagctgatgaaaatcaaggaatttatatgatgattcaagagtctgaatgggacatagaatcgcaaaagctgtttatcaaagcttgtcatgaagcattaaaggtaatatacgccaaaatttattcccgaaccaaggaaaatgaatatgctcaaattgaacgagaaaatacgaaaattatatctcaactaggacgctgtactaatgctgaaaattttagaaaatttattgcggaattctggggcaaagcagggcaactttcaattttagaagaacactgggaagaattactaccaataacaacaggtgttaggcattggaaaatgtccagggatttaacttttattgcaatcgctagctatcccaaaagtaagaacatcaagcctgaatcatcagaaatatctcagacaagtagtgaataattagaattttttctattttcttaaatccatgattttaaatttttaggtaaatactcaatgtctcatctgtttggtaatattttaactatttatggtactgctgctaataatcgaggtgaaaatgaaggtaatattactaccctacaaaaaataaactggaagggcgaagttcacaccagtgtctcagcagaggctattcgttgggcagtgcgctactactggcaaaataatggttatgaagtaaatcgacgttgggatgaaaatgcacaaccactggctgatcatatctggcaagacacgaacttcaatagtgttcggttcattgatgatgatgttttaggattcatgcaggcagaggcagcaagagttgaagcaggtgagcaatctggaggtgaagtccaggaagcatccgcaggagaaattcaaaatgtgtctcaagatgaagagacagcaagcccaaaaggcaagggaaaaaaaacttcgcaacgcaaaggacgagaaagatcacaagggagagttaccgctaaaagaggtgtactagaggttactcgtgcagtatcattgattccctatgggggtaacattacttttaatgcagtcagtggaacaaaaggacgaacttctctttatggtacagaagttcatgctactcgttatcagtatggttttgcaatgacacccaatagtctccaagacaaatctcggatcaatgcagttctagatggcttaatttcaattggagaagtaggaggaaatcatgcccggtttttatatgatttttccccagatagtattatattacgctggacacatgatttctctcctcggtttctctattgctttgaacaagatgaatggggaactatatcagttccagatttagtcagacgggtagaagctgaagatattgacccaaaagaattatggattggaggagcaattgctagagatttaggaaatttgggtgcaaatgtgtttccaggtgtgaagaaagtagtagaagaattattaaaggtcattacacgagacttacaattgccaccaaggagttcataatgtgacgactattgctttgaaagtagaagtccctatcgcttgttttcgccagtctcgtgctagggagtatgtagaaacctattcggtaccaccaccatcaacagtatatggaatgttactgtctcttgtaggggaagaaaacagatatactcactgtggagtcaagctggcgatcacacttgtatctgaaccagcaaaatcaactgtaattcgtacctttcatcgattgaaaacgaagaatattcatgatcctaaaaataataaacctgattaccaggagttactgactaatattgagtttgtagtttgggtagaccctggggtagataaagctagaccaaatttagtgcaacgtttagaagaggcatttactaacccagcttctattaatagatttggtggactatgtttaggtgaaagtcgggatttagtaaatgatattaccctcttaccaaaaaactactatgcagaatcaatgcggtggttaatcaaggatgaacacgggttgctaactttaccttattgggtagaccacgtaggttcgcaaggaacgcgatggctgcgttacgagcttcaggaatgccaagtattgcaaccgcctgaattatcttggacttcagttcaaagtgattagggggttattattttattttgcaagtacctttgcttgagaagtctaaataccatgagcaatagccttgtcttcttatttgacaagccttttagggattttatacgcatccaagatgcttgcgatttcctggaacaccgtcaacacaagagttgagggctttaacaactttccttgtgctatgaatagctattaatttttgcaactggactaatcttcagtgatagatgcttgtaaaattgaactagagcttaggcgagtcaacaagtttggcgatcgccgtgcttcaacattagatgccattaggcgttgagcaggcatcgtgcggggtgagaattgggtttacgaaaatgtgcttcaacattagatgccattaggcgttgagcaggtagttagtaataattaattctttgcccttttttacttgtgcttcaacattagatgccattaggcgttgagcagagtctagatattgcaacattaacattctgagggttctgtgcttcaacattagatgccattaggcgttgagcaggaagattttaattatcgccatgtcggggaaatagaagtgcttcaacattatcagatgctgttaggtgttgagcaaaaaaataggctcaagtatttgcgcttaatataaatacgttctcactgagtacaaaactagtcatcttgagtctcgtctttccgagaaattttagcgataattttttcccattcctcaatttgcttctgtatttgtaatttgcgatcgctcaactctttattatcattaacagacaaatcctgaccactcaatcgctcacggtaaagagcaatcagttcggttagggcttttttattgcgtgcttatcctcctgagtcagagaactcacattgactggaatcagctttctgcctattctctccacaaattcagaccgacttaaatccatgctgtcggcgatcgcatctaatccatcaatcccggtaggagttagggctaggttaactcgtttcttcacctcatcgtacagttctggtacgtctttttgacctttttgccctttcttacccattgccgtctactttttacattcctcccaatcatagtaaacacattcaacactttaagctaaacattaaaactgtttctaatctttaggtattgtgtaatgcctaatttagctagataatttaggtattgtgtaatgcctgaataggcatcttgtaggaatatgagtaactaatttcaccgtttggcttatcaacatctaaacggcttaattttcatgggcaatccttaattgctgcttatctaattcaaattaataaaaatctgctaaataattcggtcatttttcacaactatatggtgagttttttccctaaaccttatccagaggaaattttatatagtgtcattgctcgttatcacattagaagtggcaataccagtccaaaaattacacttcaagagttatttaattcacaaactactgtagcaacagtagatttaccatcgaacctgaataatttaattcaaaatctacaatttatttcaaattaccaagttgaggatttgatatataaacatactctctaccctttatatagccctttcttgtctgcaaacaggtctagtcaggttttggaatctatgaaaggagattacggaggagatattcatacaagagccggaattatggcaagctcaattactatgccaaagtattttcggttttgtccaacttgcctagaagaagacctaaaaacttatggcgaaccttactggcatcgcatccatcaaataccaggggttttagtttgtccatttcatgctgaagttttgcaaaatagtctagttcccttacacggttttaataagcatgaatatcatgcagctagtacagacaactgttctattactaaagaatcaactttttttagcactgacatactgaaaaaactactgttattagccaaagacttttggtggttactcaatagtgaaataccttgccaagaaccacagtggtttcgcaaacaatataccaatctgatgattgagcagcgtcttgcaactgcaacaggtcgaatttatcagaaaagactactggataattttttgtttttttatggtcgagaaatgctcagtgccgttgattcaatggtaaatcataatgactcaaacaattgggttactagcattgtcaggaagcataggaaatctttccatcctcttaggcatttactgataatcagttttttgaaaacgtcagtagaggaatttttcaagcaagatactgaatacaagccttttggcgaatctccttggttgtgtctcaatgctgccgcagaacattatctacaaccagtgattaccagtttagacattactttatgctgcgatacaaaaaagcctgtcggtactttttcttgttcttgtggaatgatttattgccggactgggcctgatgaaacagatgaagacaaacttcgcattggtaaaattaaaacatttggtcaactatgggagcaaaagctgaaagagttagtagaagttgaaaagttgggtttgcgggagacagccagaagattaaatgttgatgctagaactatcaaacgttatgtatctcgtctgagactgacaacttattggcagactagaaaggaaaatgactctgtaggcttacaggaattaccagaaatagatcccaattttgcaactgaactcaaattgaaacacaggcaaaattggatggttttacaagtacagaaccctgaagcttcaaagacaactctgagaaaactagcaccagccacctatatttggctatacaggaatgacagagaatggctcaatcaaaactcacccgctttacaagttgctgttccttccgttgtgagagttgattggcatgagcgagatcagcaaattctcactcaggttcaagatgcagtgcgatcgctacttaacgctgagataccagagaaaatttctattagtagggtagggaaaactatcggcttacttgcattactcgaaaaacatctcgaccaaatgcctctaacaaaggcttacttagaatcagtagttgaaacggtagaagaatttcaaatgcgccggatcaagtgggcaatcaagttgcttgatgaatgtggagaagaaattatgcgctggaaggtagtcagagttgctgctttgcgagaggattgctctgaaagggtcagtgcatttctggaaagtgaacttgagaaagcatacaagaaagagaggtgattcaattcacctctccaagtgatgggataaatgctttgtgtgttaggccgcgtcggctaccagtaagccattcatagcgatgatgatgatttgtgctgctaaaggagtatcacaacgtggtctatcgtcgctattacagggttgtgctacccatttaccctccagattttggtagaaagtgccgagaagttgataaccattccaaacacggtaaagctccccaaatacttcagataccgaatcaatctcaatttctggtgctactgggtcagcaagctggtcaatatattcctcaaactctaattgagctaaggcttggttatcaaattcatcttgtacaatcatcatgagtaacctcattaatggggttgaacaaaaagcgatcgcatttctcttggcggagggcgatcgcctttttgatatcatcccacaatatggctactttagtagctaaataataacaaatgtatctaaagctttaatttgtcacttcgactgtgcttaaaaaattgcttaccgatatttccaatgcagtacatatagcttctaatacttttagggaaacactttcagactcaccatattctaattttttcaagttttgatgggaacattcaccacctctagcagcaatttcatcagctagtttctgacgtgattttttaccacgcaatcgctttaaaatgattcctctgtcttgattccagacgacagatacaacatctgcaacataaatatacgtttttgtttctttttcacccttattgcccatatagtagctacaacagtaaacaatacataaattttgagctactatagtttacaaattattaggatttaggtatctcaaaacgaagttgcgcccggtcttcaggatgagagcgttatcatccaagaagatacacaatgcaggttttgctaagagaatgtgtcatagccaatattatgagtaatcgcggattccttaaagaggtagcaaacaaatatatattaggctgattatgttaatttgctaaaaatgttcagttcttaaaaataactgaacgtttttatctaagtataagtacgagcttattttattagtagattattaaattaataaattaataaattaaataattacttgggctatgacaccgtataaacacggttttaaaatcaaaataaggatttaaagaagaaatatctataaaagtatttgcggcaaaatgtggcggttaagagtatatgcaaatacaataacttgaatatgttttcctactattatttgtcataagtgtatcctttatggcgcaaaccatagtaatacccacgggcattcctatcataacccttcgcctccaggtacttgtattggctttgggggaagtttcaacatggtggcgaactcaatatttttctgctgctggactgagatttttagaaagaatctacccacgtacagcttttgccgcagctatcaatgctgctagtacggcagctcgtcctgttcacgacgctagtactggcagtaaccaaatctaccatctgtttcgccttcctatttccatagagcgggaagttcaactattactaacaaaggcacaaggagcaagtttggcgttagaagtcaaaccagcacttggggataaagacaagttgatgacaatgttagatgcactaactacagaaaaatctaaggtagcggttggtgcattacgtttaggaagcagtgcagaggcataccaactagcaacctacaaacggatggcaggagcatatctaagaggttttcaaacaggcgatcgcgtctttccctacatggaggcagaccaggagtgattgttatgacatcgggtgacgcagtgcaaaagtcacctcgttacagtattgcgattgtctgtaagggaactgcaatggtgaacgagactcgccatttaattcaacagtggcatcctacagaaaatgtggatacatttgttaaacgcgttcaagctgaggatctgctgggacgacaaacagcccagcgagcaaaggacattgcccgaaaagtctttgcagcacgctttttgctaccaaacaacaaacctgctcgactccttaaacaaattgttgaggctggtcttccccataaaacttttgtagaaatactatttttatttgcgtgtcgtgctgataatttattgtatgattttacgctaaatatttactgggcagcagtagcacaaagacgaaagaaaattttcactcaagacgttttagacttcttcgagtcagctgctgttgatggacgaattctctctccttggtctgagggaacgcaggtgcggacagctaggggattgttagcgacgttgcgtgatgttggttttttacgcgatgttagtcatggtaaatcggagcgagagattattccctactacatatctgatgaaggtgtagcttgccttgcacgctatctccatgaatcaggaatttctaactcagctttgtgtgaacaccaagactggaatatttttggtctcaaccgtgaacaagtgtgcgatcgcctagatcaacttggagaacaccgtggattaatcatacaaaaagctgggtcagtagtcagcatcacttggaaggtcaactctatggaggaacttattaatgtcctctctcgatgaaacttttgaattacttttacaaaagctagaccgcccagacaccctcaatgctgctaaaagtgacccaattttttacttcgtttacaaacccgaagatatgctcaccctaaagcagcgtttatcgctttggaaagctaaattaagggagttgaaatttgatgttgaaagaatatcgctctccgaccttttgtggcagattattgacgaatcagggcggtgggaagactggctagaaattgaaccttcagcagaggttgaggagttaaatcaagccattcgtgatgttttaactagtcagagtcgtctaattgaagaggtggcacaacgaattaattctttgatgcctcccaaaattgtttttttaactgaagtcgagatgctgcatccttatctacgctctcggactctagaaaacaagcttcatgatcgcgtgccagtaccattgataattttttatccaggttatcgggcgggacagtacggtctacgctttttaggtttccacgcagaagacagtaactatcgctcgataatcgtcggaggcatttgatggcaacaattcaatctcttttttccagcagtagacctattgaccgacagattgaaaaggtcatcgattattctgcacaggatgaagacagactagctacagaaatttcagaatatgagattaccgataatattgagcgatgttatcggaggtttctggatgtattttcggaaggagttcgtggtggacgcgtaactgagactggaatatgggtgtctgggttctatggttctggcaagagttcttttacaaaatatttgggatttgcattagaaggagagagaaaaaaagtacgtggcaagccatttctggaacttttatctgagcgctttgttaatcgcaatactatccaagccgaactactaactctagttagacaacattccactgttgtcattttccttgatttaggttctgaacaactttctgaaagtgctgccacgactgtttccacagtcctttattggaaagtattgcagtgggcaggtttttctaaagaaaagaaactagcacaactggagttaactttagaacgtcaaggaaaatatgctgaatttcagcagctatatcgagatacgtataacaaagaatgggaagaaattcacaatgatcccttactaggggttagtcgcgctgcggaaattgtcactcaagtattacctaaagaatttcctacaagtaaaaactttcgagaattgagatttgatctggctgataatttgcgcgatcgcaccagtgaaattattgatttagtgcgacagcgaaccaaatgtgaaaatatcttatttttactcgatgaagcgggtcaatacgttgctcctcgtggggaattgattcttaacttagatggaatggctcgtaatttcaaagagttaggtgaaggaaaagtctggataattgccacaggtcagcaaaccctgaatgaaattgtcgaaaaagctgcctataactctgctgaactgaacaagctgaaagacagattcccaatctctattcaccttgatgctagcgacattagagaaattacttatcggcgattattgacaaagccgcctgataaagaacaattattaaaagatttatttgctacccacggtcaagcggttttaacacacacacgactaactgaaacagtactttataagaatgatccgaacgctaccacctttgctcaactttatcccttcctgccccagcactttgatttgctgctggagttgattcgcaccctggctcggtcaacagggggtgttggcttacgttcagccattcgtgtgattcaagatgttctcattgacaaaagccgtgttttgccagcaaacactcttaaattagcagaacgtcctataggtgttttggcttgtgtggatgacttctacaacactttgcgcgaagacatcaacaaagtatatcctcacgttgtgagtggggcagataaagttgtcagaatttttcctaatgatgagatcgcccatagggtagctaaagctgtagctgctttacagccgattgatacctttccgcgcacagcagaaaatattgctgctcttctttacccagaattgggttcgctttctttattcgatgaggttcaacaagcactcaagagaattttaaatgagaaggagtgcggtttaattgaagaccctcaagcaggtggttatgtttttctcagtgaatctgttagacctttgcgagataagcgaacttcctacatccctacgtctaacgaatgctctcgcattaaagaagatattgttaagcaaatttttcgggatcaaccatccacaagacttgacaatattaaggaagtaaaagccattgtcaaactagagcataagctagtcgttggtcgggatgaaaccatcgatttcaaattagaattcgttgatgctggcggattctccaatcgccgtactgaattgatggcagaaacaaacagtcggcgagaattgaaaaactctattgtttggttggcaaaatcagacaacacagtagatgaactactgcaagaaattgctcgttctgagtgggtagttagagaaattgatgagcgagcagatgataaaggagatgtggcacagtttttgcgagcagagcggcgttcaggcgatcgcaaccgagacgaagtagcgaaaaaaattgagcgcttgctgatggaaggaactttgattttccgaggaaaacctacaccagttagtgaggcaggacagactctaaacgctggagcgcagacggttctcgcaaatgcagcaaaagaagtgtttcatctacacaatctcgcagctattcgaccagcaacggatgttgctgctaagtttttgggcgtagagcgtttagatcgcattacagagcaacttgatcctttgcgtttagtgactaaaaaagctagcaatactcgtgttaatgtcaaccattcagctttggcggaagtgctgcgagtgttccgggcaaaagtagatgagtctggttcaggtcgcttgcaaggaaatttcctacaagattttttctcggcagatccttatggctggactaaggatacagtccgttacttatttgctgcactattgacagctggtgaaattgaattacattaccccaaagctgatggcccactgcggacaccggggcctttagccgtggaggcaatgaaaaatactatggaatttagccgggtgggagtttcattgcgggatagcaaaccttccattgaagctttggagcgggctgcacagcgtttagaagagctttttgctgtggaagttgtaccgttggaagacaatatcagccgtgcagtacaaaaacagatgccagagttattagaacaaattgcagacttacctgttcgtctacgtcttttggatctgcggggagaacaaaaggcaaaagaactgcaagcaagtttaacggatatcctcagaggcgatgctagcaatgcaacagcacttttaggtagtcaaaattgcacaattcctgacgatatccaatgggcaaagcaaattgttaatttactcaacaatggcgctgaaagagaattcaaagtagcacgatcgttactcaataacttagatgaacttaaggatttattttccaagcaggtgctagaactgtcatctcaaggtaatcgagaagctatttacaatgtttttcactcagaacggtttcaagaacatctaccagagttgcggcgagcagttcgagaattgacggggcaattagaagaacgctacacaactgaggttgcagagtataaacaagacttacaaaaagccttaaacggcttggagctagagccagaatggttaaaactaactgacgagcagcgaaataatattgccgatcgcttgcagatggatgttgccgaacaaattgacggacagaatccagtgcgatcgctccaggtgattctcacccgccgtttaggtctaactggagtagttgataatttacggcggcagatacaacaatcagtgccacatgaggcggaatcaacaaatgggctagcaggtacttactctgtagaaaatctacatgaaacagaaagagtgaatacacctactggtgaatacactgtagaaacagcaattgatgcagccgaatttgtcatgccagtacttatagaaaattctgagcaactggatacttggttagcttccataagaggaaaactactagaaattttgaatttacgtaagcgtatacgcattaagggataaatatgagctttgatcgagaaaccctcaacgccttagctagaatggttgcagcgtgtaggcggcgactgacggaagatgtagaaaatcaactccagcagactttcgggttgtatgtagatggcacaattcttcccttatcccaattgacccatctgacagaggagcaaaaggttgcagcgcaagcattacgtgaactgctaaaccacttcaaagccagcgacccacaaaaaagctatcaatctgcttacaatcgcacaattctggaaatctcctttaccatcctcaaccgtttggcagctttgcgcttgtgtgaggagcggaatttagtaattgaatgcgtccgcaaaggaatggagtctgacgggtttcgcttgtttgaaagacttgccaatggcagtttggggagtcgttacgaaacctatcgggtgtttgtggagtccatgtttgacgagttggcggtggatttgggagtactatttgaccgcactactccacagtcaacgatttttccgagcgatcgcgtcttaacagaagtcttcgatttgctcaatgatgtcacactgcaacttgtctggactgaagatgaaacaataggctggatttaccaatacttcaatccacctgaagaacggcgagcaatgcgggaagcgtcccaagcaccccgcaatagtcgggaattagctgttcgcaaccagttttttactccacgttatgttgttgagtttcttacagaaaatacccttggtcgcctctggtatgagatgcgtcaaggagaaactcgaatcaaagatgaatgccagtattttgtcgaaaattctgagcaattcttagccagggatcgtaaagacccccgtgatatcaaagggctagatccggcaagcggtagcggtcattttctcctttacgcttatgacttatttgaggtgatttatcaggaagcatgggaagatgaaaattccccgccctcagaatttacgggtcgaaagttgcgagaagattacccaactttagaagagttgcacagacagttacctgtattaattcttaagtacaatttgcatggtattgacattgatgcccgtgcttgtcaaattgccgctcttgctttgtggctacgcgctcaacgttattaccaatcgctaaatattaaactaagtgaacgtccacccatacaaaaaattaatattatctgcgctgaacccatgcctggggaagaagaattactcgcagaatttatcgatgagttgaatcagaaacgtccaccaattattggtgaattggtaagaaaagtttttgaaaagatgaatttagccggagaaactggagcattacttaaaattgaagaagaaattagagatgagattacacaagctaaaacacaatggctagctgaatataacccagagcaaccaactctattttcattaggagaatattcagatgttaaccaattaagtttatttaagttcgccggaattaccgatgagcagttctggactcaagttgaagactgggttatagaagagcttcagaactatgctaatcgagcaacgacagggcaagttttccagcgtcagctattttctgacgatgcgctacagggatttgcctttgtggatatctgccgtaagcagttcgattttatattaatgaatcccccatttggtgaggtaagtattccttcaaaagaatatattgaagaagtttatgaagataccaagggtaatgtttatcaaacctttgttgaatgtttccaagacaggctagtagcaggaggattcttaggggttatttctagcagaacaggctttttcttggcgcaatcaagcgattggcgtaaaagaatattactgcgtttgtatcgtccgttattgttagccgatttaggttatggtgttttggaagcgatggtggaaacagcagcctatgttctacgtagtattacaatagaagaagataaaaacttaacattaaatcttgttccacaactattagaaattcctgtagatagacagagatgttttagcattcccaaatatcagaaacatcgaggcggcttaaagcggcatcaggctaatcaagaattaaaaaaactgcacgaaaatggttatatatcattgacactaggtcgttttattcgctataaagctaattttgcaaaaattcaacagtctgatattcctagttatttagcatatcctaatttaatttgttttcgactgttggaggaaggagataaaaaaagtgccttaatagatattttacaaaattacaatgactctagatattttgtagtttcgccagaaagtttttcaatggttcctaatacaccattttgttattgggtaagtaataatattcgccagttatttactaaactgccgcaatttgaaagtaatggtagaatcgctcaacacggagcatcaacaaaaaaagatccacgatttctacgtctttggtgggaaatactacctaataaaatagtaactggtacggtagaaacaacaccagaacagttttgtcaacaaacttttgaggataagccttgggtcttatttgctaaaggtggagcttattctccatattatgcagacttacatttgctaataaattggaagaataatggcgaggaaattgaacaatatgttttatacaaatatccatatctaggcagaagtggtgcagattggattctacatagggagtgtaattatttccaagcaggcttaacatggactactcggactactagcaagattagttttcggaatttgcctgcgggtagcatatttagtagtaaaggagcagcagcttttactaaccatgaaatgtattggttggctattttacaatcgtctatctctgaagctttaattagtttgcagcttgcagcagtagatgcagcagcccgctcttatgaagtaggtttaattcaacgtacacctgttcctcctaatagttctgatttccataaggaaatattagtaaaattagcagatttttgtattggtattaaacgctctttagatacagtaaatgaacatagccacatatttcatatacctgctctgcttcaagcattaggtaataacttaactgataagatttttaattgggaagataatattaataaatccactaaacgattaagtaagtatcaagaacaaattgatgatgtcgtcttccaactttacggaatatcaaaaacagagaagaaaagtcttgaaataaatattaaaaacaaaaatataagaattgaagataatactgaagatatagacgaagaagatgaaactgcactagcagcagacgcaaaacagctagttactgatttactttcttatacactcggttgctcttttggtcgttgggatatccgcttttctacaggcgataaatcagcaccagaacttcccgacccctttgcaccattaccagcctgttcaccgggaatgcttattggtactgatggtttaccagtttcagaaactccgccaggatatcccattcccattgcttgggatggcattttagtagatgaccccaatcaccccaacgacatcacccgtcgtgtccgtgaagttttagaaattatatggaagaacaacgccaataaaattgaacaggaagcttgcgaaattcttagtgtgtgtgaactgcgggattacttccaacgtcctacaaacttctttcaaaaacatctcaaatgctataccaaaagtaaacgcaaagcaccaatttactggtcactttctacaacatctagttcttacacagtctggatttattaccaccgccttacagaccaaacgctctacacaatcgtcaaccgctaccttgaaccaaaaattgccgaggttgagcgtatcactggtggcttagaaaaagaattagagacaaaatcgggacgggaagcttcacaagtacgcgataacttacacgcaacacgcaaattcctcagtgaattgcaagatatgaagcaggaacttttgcgcgtcgcacagctaccttacaaacccaatctcaatgatggcgcaattatcactgccgcccctctacaccgtttatttgctttgcgacagtgggcgcaagaaaccaagaaatgttgggaaagtttggaaaaaggcgaatacgattgggcgcatttagcctacacaatttggacagacagagtgcgggaagtctgcaaaagggataaatcaattgcgatcgcccacggacttgaagaagtttaccaagcagctaacccagaaaattcccaacccaaaacaaccaaaggcagaagccgtaaaaagacgacacaggagtcagtataatgagcgtctcagactttgtttgccgacaattacaccaaagattagaacagcaccgtattgtcgtttggtacgatggcaatcgcgcttttgctgattttattcaatccttccaagctgactcctgtgtagtcgtatctgctgttgagtctattttgaactcccgtcgtcaagccgaagccgtctaccgccagatgaacgagtctaataactcagccaaagctgggttgaatctcctcatttacgtaccccgccagcgcggaacaatagaagatgagaagcggcaagatccgtttgaagttttcgcatgggctggtgtggcgtttggcgacaaagaagcagaacagttagaatctcttgctagacaagcaatgccagaacgggctatagaaattactcggctatttgcccaaggcagtccaacgctatcgatgttggataaattacagttccatcaccgttcgcctttactgttagaggcgctaggtactgactcaccatcagaagcgatagccttagtcctctgcttagaggagaaatcacaacgaattgattctgttaaaggttgtttaaaagaatttttgccgttattggcaacggagattggttttaaacctgcatccaaagctaaatcatggacagaaattagaaagcaactcattcagtatgtgctgtttagcgaatttgccctcgacctgccttgtgacctgccagagtcattaagtaccttacccagagccaaagccagccatgaattagtgatttttgcagtgtgcgatcgcatgagggatagttccgacttgcgatcgggctatgttcagctagcagtagaagtagaaaaccagttgcaccttggcaatctcacagaaaatatagctcaactaggtactagagataccttcccctttgcagaaaagcgatacctacagcaagtgctggagtatgtgcaacagaataacttagcagatgcccagaagattttaacccaacgccgtcattccatctggaacaaccacttggaacgaggtaatatttggatgatagtagagcgttgtgtgacatttctcgccacaagtaatgctgtagaagagttgtcaaaaaaatctttacagtcaatgtctttacgccaaatcatctctgcatacgcacctccagatcgctggggatgggcagaactagaccgacatcagcgactatttgaacagggtgcaaccgcctatgcaggagatgagggagagatagaatctttagtcgaattttgccgccgacgttacttagaagtggcattagcaattcaagactgcttccttaaatgtgttcagaaagaaggttggtttcccgaaggagtgctgcgataaacccaaatatttgaccagtatgtagctcctctactggaacagaacaacaaaaaagtcgcattttttctagttgattccctgcgcttcgagatgggacgagatttggcagaagctttagaacagttgggcgaagtcgagattttaccagtcgcatctgttttacctacaactacaccttgcgggatggcagcactgatgcctaatgctgatggtacgctcagactggtggaatcaggcggagagctaattcctgtcttaggtgaaaggaaactggaagagtccaagcaccggatgcgactgctacaggaaaaatacggcgatcgcttcaaagatatcgaaccggaagatattttcagcaaaaaggtaaatgccttgggtaagcagttagcctctgtggatttattagttgtccgcaccaaagaccctgaccgcattgctgaaagtattggcaacatgaaagcccgaaaatatctttcggatatagtcgctgaaatggtaaatacaattcgtcgattatctacaattgggttcacccattttgtcattagtgcagatcatggtcacgtcctaattgacgaaataccattaggagatgtaattcccaaaccagggggtgagtggttgaaaactaagcgcaggagtttgcttgggcaatttatatcgggcgaatcttcaagtgtagttatcttcaaaactgagcaggttggtatccaaggcgatgcccaagacctctgtgttgctaaaggctttaaagttttccaacaagttgaaggttacttccacgaaggcataagcttgcaggaagcgatcgtgccattggtaattttgcgatcgcaaggtaagattactgctcagagtaagccaaatattttcctgcgttataagtacgattactttaccaataaagtcattaatctgcgagtctgctatgaatatttgctaccagaagcaatacaagtccgcattgaagcttacgatggctcaacagcaaaagccaagcgggtaggtaaagcaaccgactgtgatgctagagacgaacgaactggtgaaatcatactccagcccaatcaagaaacttcggttcctatcttactggagtctgacgacatttcatgctccaaaattgaaatacgagtaattgacccacaaactggcgtaatttgggcaaagttagaactggaaaatcgcttactagattaagaatgaactcatgtttcaagatgccttagaccaaaaagttaatcaaacatttccaggaaaggtagtacgtaaggacttgctacatcaaatcaagggtggagaaaatgtaccatcttatgtactggaatatcttctgggaaagtactgcgcctcagatgatgaagatgaaatccgcattggtattcaggctgtcaaagaaacactacaaagcaactacttccgccacgatgaagctaacaaagcccaagcgctagtagaacaacggggacgacatcgatttatagaccgtatagaagttcgttatctcgccagtgaaaacaaatattgggcggcaatggatcactttagttactccagaattcatgttgctgaccgattttatcgacaatacgaacgacttttagaaggtggaatttggggtcttgtagatgttgaatttcaaccaactgaagaagaaggtgctaaagccagtccctttcacattgctgacttaagaccaattcagttagcaagatttaacgttgatgaatatgcagaaggtcgtcgcagcttcagccgcgatcaatggattgatattttgctgcggagtgtgggtttagaacctacccaaatagaacaacgtttaaaactgctgttactgactcggttcattccctttgtcgaaaaaaattataacttcattgagctagggccacgaggaacaggtaaatcctatgcctacagtgagatgtcgccttacagcatcctcatttctggtggtaaagccagcactgcaaaccttttctacaacaatgcccgccgtcaggtaggtttagtaggtcattgggatgttgttgcttttgatgaagtcggtggtttgaaagttaccgatgcggatgcaattcagattatgaaagattacatggctaacggtcgtttcagtcgtggaatcacccaagttttagctgacgcgtctttggtatttgttggtaatcttaaccaacctgttgaaactctagtccaaaatactgcaacagacttatttcagccattaccaaaagaatttgatctggctttacttgaccggatgcacttctacttgccaggatgggaagtgccaaaaaactccaaaaatctgcttacagaacactatggttttgtcactgattatttggctgaagcgtttcgtaccctaagaaaacaaaatcggtttgatgaagtcgagaaagtcttccgttttggttctcatgtagaaggacgagatgcgatcgctgtcaaaaaaaccgttagcggattacttaagattatccatccagacggagaatggacaaaggaaaacctgacagaatatgtagaactagccttagaagggcgcagacgagttaaagaacaactcaaaaagcgaggttctttcgagttttataaaaccagtttttcttacatcgaccaggaaaccgaagcagagatgacagtgggtgtaccagaacaaggtagtgctggaattatctctcaagaacccttacccccaggcacagtgtacaccgccacagcagatggagaagcaagggttggtcttttccgcttggaagtagcttgtacatcggggacaggaaaattgcgaacccctacaggacttgacaaagctttgaaagaatcattaaaccgcgcttacagctatcttcaaagtataaaggaaagaaaaggactcaatgaaactttgggattgaaagatatttacgctgaagctgtggacttgtctggaagtcgtgttgaatgtccttgtggtgtagcattttttgtcgccatcatttcagccatacagaatcgtcaaatccaagctggaactcttatcttgggcgatttaacgattcaagggaatatcaaaggccctgcttcaattgttgaatcattacaaatagcaatggaaagtggagctttacgagctttagtaccactttctaataaaagccagtttgcaggtttaccagaagagatagtcgaaaagctggatattgtgttttacggtgatgttgaccgagcagtaattaagagcgtcgaaatttaagagactatccctattatgtcactctcgaaaaacaataattgaaacaagattctaaacctttgatttaattaagcttcagcctttattttctaacaataactaaatttatagccaaaatcagaaaattaaagccctgaaagctttccagcgattcggttctcctaaaatgacacaacagggctatttataaaataaatctttaggctacaagacgacttagcataatcctactttgagaaacctttaatgtagctgatgagatgttgatctatattgtcgatatcacctttaagctactgaaaatcgagttctgaacggttatgaaatgcgattgctcgtgaccttgtagatgcagttcaccgcaattttgctatcgactggccacagaaataactttttaaaactaacctctgccgattagcgtgtgaggtcgagaattttgttgtgcgatcgcctcactacgaacaaatgctaacttttttacacccctttaaatttgcttgtttctagtatcgctgtttctacgatttttttgagattaatagcacctccaattacagtagaaatactgtgaatttagcttttattgacttggtacttagctaaaatactattcttctattggagtattgaggagcgctcgcgcaataggaaaagcaagacacaatatttatttataaaaaggaaataattcagcttaaaggtttcttaatggaacctgacgggaactttcaatctttcatcaacgtgattatagtaagctcaacagtaaatatacgatttatagttatttagttattgttaaccttcaagccatctaagctaaattacggatgattaacccccagctagacaaaactattgagcgttatgagacggaaattgagagactgaaagtgactgacacggccatagatgccgaacaagttttggaagtgttaagtgccagggatgctgtgcagttagccttaaagggtgcaacattcattcctagcagtaggctaaagcaggttctagagttagatgcagaactcaggaaacaagcagtacacattaacaaagttatcaaagctgaacaattcgcccagtggcgagaaagtacacatcctcctgctgaagcttggtggtggcggttagaaaatcttacccctcaccataagtgggatagatttgattggttgtggaagggtctagtagttgctggctggactgctaacctcagtttattaatcaatattgctactcgctttttgagcgggggagcaggattattgggagcttctgcggttattttgccgagtattctggcattgctacaggcaagtagtgaactaactcaagcaggtaaacaagaatttgacaaattactcacaaaactgaatatcccccagcacttacacgaagaagctaagttcggatcaactttattaatgacaatctttttacttggtttttggttggctctaccgtcaatttcccaactctacaaccgtcaaggactcagtaattacctacagggaaaattaggagcagcagaacaagactaccttaaagcaatttccttggatgccgataatgcagatgctcattataatcttggcaatctttacgaggatttacaagattttgagaaagcacggaagcattatcaaatcgcagtgggaagtgacgttcctgatgcttacaataatctagctcgcttatacatccaggataaaaaatattcccaggctagagctttgctacaaaatggactagtaaaagctaaagaccttcccgaagtcagatacagcctgtttaaaaatttaggatgggtaaggtttgagcaaggcagatatgaagaagcacaactaaacttacaagcagcaattggtattgcaaataacccagaggtagcaaagaatatccaaaatactggtgcggcgtattgtatcttagcgcaggtaatggagcggcaaaaacaaccggccgctttggagcaatggcagaagtgctgtgaacaaggttctactctcaatgctgatgaagacacatggttgcatctagctcggcaaaaattacaaaaggcaggaagagtatgcaaatagaccggaaaaagattagtttcttagccattgtactaagcctaccttttatgacggtagcaacacctgggctgagtcaacagaatagtctaaattttatctccgaagttaagggagatgtcaaaattcaacgttctggacgcaagaaccatcaaaaagcatatggtggtgaactactagattcttctgattctttgcgattgggaaaaggtgcttctacaaaagttgtatgtaataacttatccatttggaatattaaatctccagggaaattttcagtctctagtggttgtccatccactggaaaaccagttcttataagaaatggtagtgaacgcgctccgactcgcgctcccaatgacccgacaattccctacatcattagtccgcgtgatacagcaattttgagtggcaaacctactctgcgttggaatgctgttaagggtgcaaccagctataaggtgacgttacgcggccctggtgttaattggacaactaatgtgaagcagtcagaagttgtttattctggtcaagaacctctacagccaggttttcgttatcgggtagttgtgactgctgataatggagcaacttctgagagtgatgcacctgttggattcaatttactgagtgagggggaggctcaacaggtaaaagctgaaattacacaattacaaaagcaaccgctaactgatgaatctaaaaatcttgctttggcatatttgtaccaaagcaagaatttaaatgctgtggcaattgatttattagcaggcttagttaagcaagaaaaccaaagcacagcagtttatcagcttttaggtagcctctaccaacaggtaggtttacatcgattagctaaagaacattacttaactgcgctcaagttagctaagacaaacaaaagtctggaagcgcaagcaatcatccaaaccagcttaggagaaattgatatagttcttgacaacttgcagcaggcgtttgagtcattgcaagctgctcaaaataactatcgtgccttgggagatgagcaacaagtacagcaactacagcagaaactagatgacttgaaaggaaggctaccatcatgaataacctgcgaactagacaattagtaaccctaacaattttggtagcaacagctggattatgggtgttaatgccaaagcccacaattgggcaatcaacccctcctgtttctgctgctcagtctgcggaattggcggaagtagaaagactcaatcaacaggtgattcaactttatgaaaaaggcaaatatagtgaagcaattcccttagcagagcgtgcattggctatccgtaagcaaaaactaggcgatgagcatccggatgtggcaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntagcttgaataatctggcattgctgtactggaaccaagatgacacaagccgcgctcttgagtttttaactcaaggaataacagttcaagaacacaacctcactctcaacttggctgtcggttttgagcgacaaaaacgcgattacgtcaagactatctctgctaccacagatgcttccatttcactgcacctcaatggtgctgctaataaccccagtgctgctaatttagctctaagcacagtcttacaacgcaaaggacgcatcttggatgtcctcactaatagccagcaaattctacgtcaacgcctagacccagaaagcaagactctgttaacgaagttagcaaacacccaaactcaactagccaatctattttataaccgaccagagaaacttcctcttgagcagtatcggactcaaattgcccaactggaaacccaagcaaaacaactagaagatcaactgagtcgtcgcagccaagaatttcgtactcagtcccagcttgtcaccattgaaactgttcaaaaattaattcctgctgatggtgctttggtagaaatagtccgctatcgacctgttaatcttaaagcagcacctgaagatcagtttggtaaacctcgttacgcagtatacgtccttccagcaaaaggacaacctaaaggtattgatttaggcgatgctgaaacaattgagcaaactctcatccaattacgcaaggacttgcaagatcaaaaaactcctatttctcaaatgaaacagtcggctcgtgagttggattcaattctcatgcagcctgtacgcaaacttttgggcaatactcgcaaaattttactctctccagacagtcagcttcacctcattccctttgaggcactggtggatgaaaataaccgctacctactagaaaactattctttcacctaccttacctctggacgagacttgctcagactgcaaaacaaatctgccagtaaacagccgccagttattattgctgaccccaatttcaatcgtcctggcaaattagttgcgcgtcagccttatgctaatagtattgatgcaaatacccgctctatagacctgtcccagaaagtatttccccccctccccggtacagcacaagaagccaaggccattgccagtctattaaaaatccaacctcttacagacactaaggctactgagggtgctgtcaaacaagttaacagccccaggattctgcatattgccacacacggtatttttgaaaatcccccagcaaacgaagggaaaaccacacttgaagataatccgctactgcaatcaggattagttctagctggcttaaaacctcgtcagagtgctggtgaagatgggattctcactgccctggaaacaactacactcaatctagttggtacaaaactggtggtactctctgcctgtaatacaggtttgggtaatattaatgcaggtgaaggcgtttatggtttgcgtcgcgctttagtcattgctggttctgaaagccaagtcatcagcctgtggaaagtagcagatgatgctactaaagattttatggtggcttactaccaaagattattagataatcaaggacgcagtgaagcactacgccaaacgcaactagagatgttaaagggtgataagtatcagcatccctacttctgggctgcgtttatcccttctggtgattggaaaccgatgggtaagtgagttagttttgagagctttggcgatccttccatattatcggctcaacatgaaatagagtgcgatcgcctccctcgaaactaaactgttgattcagctttgtactgggaatgcgatgcgatcgccactaaatcttggagtaaatattcccaaatccttacggggaagtaatttgcaaaattgtacaacttttttatttctttttggatagtagcctgatgaaattgtagaactttattttttcaagacttcttagtaatcaacgatataacaatccttgtagccgcgcaaattatacaactttattatatttatacagaggttgtagagaaataattcgattcaacaataaagatgttaaaaataacctctaaagaattactaaaataattgcaaatagaggttatttttattggaacagagacaaaacaggtttttcaaaggctagagtctttcgatatcaggtaaaatttgataaatctgctgccagtgaggacttatccagccaacaatacgcactacttctcgtaaaattacgatagacaaaccgcattcaaccttctcaaaagcagttgcacaatctgttaatagacttacgaaactatctagtttttgtaatttcatctttgctaatccatctgcaaattgcccaatttgagctaatactacactaatttcgcctaaactctcttcgtccatatccttacatattaaatcccaatctttcttgatttctgaaaatatctcctcagcttctacttcttgcccttgttgataaaaggcttctactaaactatataaagctactatcttttctggtgtatcttcaatcaagcctgctactttcttagctttagaacaaaagccaaatcttgctaagacttgtgttaaattctctaataaaaatggttgatcaaaaggattatccatttcaagaatcatttcttctatttcagttagcaatatatctgctctttctgcgtattcacccttataaaggctatttaataatattgttaaagcttggatttggaaaaaaccagtttcaagttttctggctattctttcggtagctaaaaatagagtattagcatttttgggtttgtttatacaattaaatgcgactgccaaatcagtcattaatcataagcgataaggctcatt

SEQ ID NO: 75 - DNA, 36 bp, Artificial Sequence
  Synthetic
ctgctcaacgcctaatggcatctaatgttgaagcac

SEQ ID NO: 76 - DNA, 36 bp, Artificial Sequence; identical to SEQ ID NO: 75
  Synthetic

SEQ ID NO: 77 - DNA, 36 bp, Artificial Sequence; identical to SEQ ID NO: 75
  Synthetic

SEQ ID NO: 78 - DNA, 36 bp, Artificial Sequence; identical to SEQ ID NO: 75
  Synthetic

SEQ ID NO: 79 - DNA, 16 bp, Artificial Sequence
  Synthetic
aggcattacacaatac

SEQ ID NO: 80 - DNA, 16 bp, Artificial Sequence
  Synthetic
aggcattacacaatac

SEQ ID NO: 81 - DNA, 16 bp, Artificial Sequence
  Synthetic
aggcattacacaatac

SEQ ID NO: 82 - DNA, 16 bp, Artificial Sequence
  Synthetic
aggcattacacaatac

SEQ ID NO: 83 - protein, 243 aa, Artificial Sequence
  Synthetic
MIAPLILYLDVPFTTFRESHAREMGKTYPVPPPATVYGMLLSLVGETNVYRHCGVELAIAMLSSPKKSRILRQMRRFKNADFSHPENVIPCYQEILSNLKCLIWVRSDEEKIQPSLRERIQLAFDHPELVRRFGCLFLGESDQLIKTIKLAREDYLEGVRQWAIRDNRGRLTLPYWVDHVGSRNTRFLRYRIEEMDRLSPPDLAWTMVQSPIGGSGGGGSGGGGSGGPKKKRKVYPYDVPDYA

SEQ ID NO: 84 - protein, 251 aa, Artificial Sequence
  Synthetic
MTAILIQSEEYVDLTFKLRGAPIPLDNGYLTYAALSRICPPLHELKSIGIHPIAGIPTRNNLLELTAQSRLKIRIYHQQIPLIYPYLAGQAFHIGQNFYQLDIPDYKPLISSESVYSRLVIIKGFQDSTNFIEAVQRQMDNLGIQGKIELLTRQDGTPQRRQLTINKEGKQFKVRGFGVKISELNPEDSLTLQEQGIGGKRKMMCGIFVPATRSKEEEETGGSGGGGSGGGGSGGPKKKRKVYPYDVPDYA

SEQ ID NO: 85 - protein, 354 aa, Artificial Sequence
  Synthetic
MMTQKKNDSNIPNYYLYGTVLTRYGLASLNHDIRRGNKTILQKGYWNNGKIHSFVGSSAIRWALRFYLQKQGYLVNRVWDEEEHINRLTSEDFDPEKFYDDDIFGFALLESAETEEDTSTTKRKKKQTKTSTPNQRMGALGMNMAVSLTPYDGAVKLGAKSGREKDSTSLHFTEYHATRYQYYFGIDATHLKDFSRILPMIDGIMNLPKVGGSSNIFNYPFCPDSLVFQWTNHFASYISYCFEYCDPKSKEAKLSQEFIDEVECGQIDPSKLWIGGTIVKDLQQLDNFESSPLNKAHIYRNRNEMIEALKTVIKRDLGLEESKGGSGGGGSGGGGSGGPKKKRKVYPYDVPDYA

SEQ ID NO: 86 - protein, 557 aa, Artificial Sequence
  Synthetic
MIVSTQPKISLSLHAADTTIMHRVGMTGLYMTLKRLEKQYPLSRQRGGHISWFLTADTIELFWEGSDFIALSWLINESFQLDDTGLIHLVGLDNDRIDLRQKIHIHEGICGVFLRLNKFYQAGEIINTELRFEEKQVEYQYKSLTWYAHQTFAEKLCEADTQQLRHDYIQITSWLYLGGIVRHARTQNTTKLEEKPEYALALLFVPVVCHYCLLHIPSEDLKERKPHRYLVVIPEIKDFEDASQRRWRLQQLETKQFHVSSLGEAGLLYYSLDDIQPEVAYYQACQVWLYEKTNKASRQRTLMSIEEIKIDKNILITYQQVQKYFKTNYQIIKYKQIFIKVNPIRSLIADNLVKGIHWWSNFWEKLVIEDSKEYLFNQLFSNREGFIIMAENSEEDKQYLIFIKVFQQAMKGNFAKIYAKTEEGKDPPIKKKVERLRAELNYCYDELSFKEYLSDFLVRGGLNKYFNEHQEEIALLIKKSPWQEIRIWSLLAIASYKPKDKLTNRDDSSLSNNQKLEEVNDDSEEEGGSGGGGSGGGGSGGPKKKRKVYPYDVPDYA

SEQ ID NO: 87 - protein, 243 aa, Artificial Sequence
  Synthetic
MYPYDVPDYAPKKKRKVGGSGGGGSGGGGSGGIAPLILYLDVPFTTFRESHAREMGKTYPVPPPATVYGMLLSLVGETNVYRHCGVELAIAMLSSPKKSRILRQMRRFKNADFSHPENVIPCYQEILSNLKCLIWVRSDEEKIQPSLRERIQLAFDHPELVRRFGCLFLGESDQLIKTIKLAREDYLEGVRQWAIRDNRGRLTLPYWVDHVGSRNTRFLRYRIEEMDRLSPPDLAWTMVQSPI

SEQ ID NO: 88 - protein, 251 aa, Artificial Sequence
  Synthetic
MYPYDVPDYAPKKKRKVGGSGGGGSGGGGSGGTAILIQSEEYVDLTFKLRGAPIPLDNGYLTYAALSRICPPLHELKSIGIHPIAGIPTRNNLLELTAQSRLKIRIYHQQIPLIYPYLAGQAFHIGQNFYQLDIPDYKPLISSESVYSRLVIIKGFQDSTNFIEAVQRQMDNLGIQGKIELLTRQDGTPQRRQLTINKEGKQFKVRGFGVKISELNPEDSLTLQEQGIGGKRKMMCGIFVPATRSKEEEET

SEQ ID NO: 89 - protein, 354 aa, Artificial Sequence
  Synthetic
MYPYDVPDYAPKKKRKVGGSGGGGSGGGGSGGMTQKKNDSNIPNYYLYGTVLTRYGLASLNHDIRRGNKTILQKGYWNNGKIHSFVGSSAIRWALRFYLQKQGYLVNRVWDEEEHINRLTSEDFDPEKFYDDDIFGFALLESAETEEDTSTTKRKKKQTKTSTPNQRMGALGMNMAVSLTPYDGAVKLGAKSGREKDSTSLHFTEYHATRYQYYFGIDATHLKDFSRILPMIDGIMNLPKVGGSSNIFNYPFCPDSLVFQWTNHFASYISYCFEYCDPKSKEAKLSQEFIDEVECGQIDPSKLWIGGTIVKDLQQLDNFESSPLNKAHIYRNRNEMIEALKTVIKRDLGLEESK

SEQ ID NO: 90 - protein, 557 aa, Artificial Sequence
  Synthetic
MYPYDVPDYAPKKKRKVGGSGGGGSGGGGSGGIVSTQPKISLSLHAADTTIMHRVGMTGLYMTLKRLEKQYPLSRQRGGHISWFLTADTIELFWEGSDFIALSWLINESFQLDDTGLIHLVGLDNDRIDLRQKIHIHEGICGVFLRLNKFYQAGEIINTELRFEEKQVEYQYKSLTWYAHQTFAEKLCEADTQQLRHDYIQITSWLYLGGIVRHARTQNTTKLEEKPEYALALLFVPVVCHYCLLHIPSEDLKERKPHRYLVVIPEIKDFEDASQRRWRLQQLETKQFHVSSLGEAGLLYYSLDDIQPEVAYYQACQVWLYEKTNKASRQRTLMSIEEIKIDKNILITYQQVQKYFKTNYQIIKYKQIFIKVNPIRSLIADNLVKGIHWWSNFWEKLVIEDSKEYLFNQLFSNREGFIIMAENSEEDKQYLIFIKVFQQAMKGNFAKIYAKTEEGKDPPIKKKVERLRAELNYCYDELSFKEYLSDFLVRGGLNKYFNEHQEEIALLIKKSPWQEIRIWSLLAIASYKPKDKLTNRDDSSLSNNQKLEEVNDDSEEE

SEQ ID NO: 91 - protein, 360 aa, Artificial Sequence
  Synthetic
MYPYDVPDYAPKKKRKVGGSGGGGSGGGGSGGTIYSEHWSLNPLEIPQRSRLFSLEPVAVGTPYAESLSSYLHRLAQAHCLTSEKLVMGEIAPLILKDEDKSELLSKNLSHLLGNSDAKPAINGMREMTEKLVTVLEELTMRQDLRFLTLLSWKGMIYDKGLFRNYRAWCPCCCEEWMQKNKTIYEPLSWSFKDVEFCLIHKQRLIEECSHCGARLPVMARLSPAGFCSRCYGWLGQEIKGEEEIEKYRVNIQGISELIALTPQLGYKPIPIELTRKLQLILLVFEQAIGKDVKLLGDLGGIMESLRIASTTNQSQPYHLVKLIIPVCEKAKISVFQLFGSDFKELGKILFGNFSLELKL

SEQ ID NO: 92 - protein, 493 aa, Artificial Sequence
  Synthetic
MYPYDVPDYAPKKKRKVGGSGGGGSGGGGSGGLASVLETYESWNLKKPLIPQRSRLYQPQPVGIGTPYIESLTGYITRIAELHGVLPGVLMTREIAPLVNKIYFQNGANRGFREIFNRSQALNGMGEMAADLVQVLQKLTLRDDLRFLTMLFWSNILTPRNLFRTRKAWCPICYQERHQNGLVVYEQLLWTINLITICPQHQKPLVELCPHCNHESPLLNWRSRPGYCSKCGEWLGANQCLKTFTDGEGSIKLQLEWQYWTANVVGELILASQCFESAPSKENITKSLNIVIDKVAENNAAAFSRLIGVPKNSLWMWQSTKTLPELNTLLKICYELEISLVEFLTPKNLITKSFTKISQKHLQLSRTPRVSPKSFDQYQVKDALLAILAGNEEPPPTMEEVGKRLGHHNRTISRHFPDLCSAISAKCRNYNKACRLKSIEKLCSEVREIVLSLNAQGVYPTEGRVCELMPNPGCFRYKQVRAAFNDARREFGL

SEQ ID NO: 93 - protein, 929 aa, Artificial Sequence
  Synthetic
MYPYDVPDYAPKKKRKVGGSGGGGSGGGGSGGKEQFEQWVARLQISEAGQKIIEKVRSSEPSRRVGGGSKNVSGRYPSRKMGVTIQFESHRVELPIIYQLEHDKDVLEFYDQPPQIKLDYQASNGRRLGVLHTPDLFVIRTNSAGWEECKTEQDLKKLAEKNPNRYFYSEENNQWHSLPGEVYANQFGLYHRIHSDREINWVLQRNLQFLEDYYRSESLVVEEAIAQSLLAIVSSQPGITLAELLNRSTGAKSDDIYTLIIQEQIYIDLNATPLAEPERCLIFRDEQTASAYRLMVEQPSVSLPAISPVVNIVTGATVSWDGKGLNIIHVGETEVILGAENNQLIELKKAIFENLVRQGKITSLQTPEKTAISTESWQRFHQASPEDQAEALERYKTIEPYLNGHPPENETIPARTIRHWKAKYLIAIQKYGCGYIGLLSHRSVKGNRQRKLPEDTLAIMEKFILEDYETLKQKRMWEVHAALVRACEEAGVIAPSYKAFTKEVQRRTGYEQTKKRQGRRAAYQHESFYWELAITTPRHGDRPFEIGHIDHTELDVELVCSDTGRNLGRPWATFLVDAYSRRLLAVYLTFDSPSYRSCLMVLRICVKRHGRLPQIVVVDNGAEFHSVYFETLLATFECTKKQRPPAKARFGSVCERLFGTSNTQFVHNLLGNTQITRNVRQVTKSVNPKNLAVWTLGLLYEYLCAWAYEVYDTDEHPALFQSPRDAFAAGMAIGGSRVHRMIPYDENFQILTLPTTSEGKAKVQVGRGIKINYIYYWSNSFRDPQIENTSVQIRYDPFNIGIAYAFVRGQWVQCISQYYAELQGRSEKELKLASIELRKHYSNHTQQSKVSAKNLAEFLASVEAQEALLEQRSYDAEVKEVFRVIEGGKATTSRNEEPKLIQVTFANTDFQADEDEAIVPETLVVYEEF

SEQ ID NO: 94 - protein, 414 aa, Artificial Sequence
  Synthetic
MYPYDVPDYAPKKKRKVGGSGGGGSGGGGSGGTKSTGFPLELLTRPATERLAYFENYTVAHPRLKEVYEILMRTIAEPAGASFIFVYGASGVGKTTLRLRVEQKLTELALPKLESDRARVPVVGIEAIAPESRYFNWKEYYTRALITLEEPLIDHKFDYGVRGISRDNFGKINVESKVVAPALRRALENALIHRHPDVFFVDEAQHFGKVASGYKLQDQLDCLKSLANMTGILHCLLGTYELLTFRNLSGQLSRRSVDIHFRRYCADSPEDVQAFKSVLLTFQQHLPLAETPNLVDHWEYFYERTLGCIGTLKDWLKRVLSDALDREATTITLKDLQKRALSVAQCQKMFKEIQEGERQLSETEADVQNLRSALGLGAKPIVLPEETPKTTRPPGKVGKRKPKRDPIGVQQDVS

SEQ ID NO: 95 - protein, 360 aa, Artificial Sequence
  Synthetic
MTIYSEHWSLNPLEIPQRSRLFSLEPVAVGTPYAESLSSYLHRLAQAHCLTSEKLVMGEIAPLILKDEDKSELLSKNLSHLLGNSDAKPAINGMREMTEKLVTVLEELTMRQDLRFLTLLSWKGMIYDKGLFRNYRAWCPCCCEEWMQKNKTIYEPLSWSFKDVEFCLIHKQRLIEECSHCGARLPVMARLSPAGFCSRCYGWLGQEIKGEEEIEKYRVNIQGISELIALTPQLGYKPIPIELTRKLQLILLVFEQAIGKDVKLLGDLGGIMESLRIASTTNQSQPYHLVKLIIPVCEKAKISVFQLFGSDFKELGKILFGNFSLELKLGGSGGGGSGGGGSGGPKKKRKVYPYDVPDYA

SEQ ID NO: 96 - protein, 493 aa, Artificial Sequence
  Synthetic
MLASVLETYESWNLKKPLIPQRSRLYQPQPVGIGTPYIESLTGYITRIAELHGVLPGVLMTREIAPLVNKIYFQNGANRGFREIFNRSQALNGMGEMAADLVQVLQKLTLRDDLRFLTMLFWSNILTPRNLFRTRKAWCPICYQERHQNGLVVYEQLLWTINLITICPQHQKPLVELCPHCNHESPLLNWRSRPGYCSKCGEWLGANQCLKTFTDGEGSIKLQLEWQYWTANVVGELILASQCFESAPSKENITKSLNIVIDKVAENNAAAFSRLIGVPKNSLWMWQSTKTLPELNTLLKICYELEISLVEFLTPKNLITKSFTKISQKHLQLSRTPRVSPKSFDQYQVKDALLAILAGNEEPPPTMEEVGKRLGHHNRTISRHFPDLCSAISAKCRNYNKACRLKSIEKLCSEVREIVLSLNAQGVYPTEGRVCELMPNPGCFRYKQVRAAFNDARREFGLGGSGGGGSGGGGSGGPKKKRKVYPYDVPDYA

SEQ ID NO: 97 - protein, 929 aa, Artificial Sequence
  Synthetic
MKEQFEQWVARLQISEAGQKIIEKVRSSEPSRRVGGGSKNVSGRYPSRKMGVTIQFESHRVELPIIYQLEHDKDVLEFYDQPPQIKLDYQASNGRRLGVLHTPDLFVIRTNSAGWEECKTEQDLKKLAEKNPNRYFYSEENNQWHSLPGEVYANQFGLYHRIHSDREINWVLQRNLQFLEDYYRSESLVVEEAIAQSLLAIVSSQPGITLAELLNRSTGAKSDDIYTLIIQEQIYIDLNATPLAEPERCLIFRDEQTASAYRLMVEQPSVSLPAISPVVNIVTGATVSWDGKGLNIIHVGETEVILGAENNQLIELKKAIFENLVRQGKITSLQTPEKTAISTESWQRFHQASPEDQAEALERYKTIEPYLNGHPPENETIPARTIRHWKAKYLIAIQKYGCGYIGLLSHRSVKGNRQRKLPEDTLAIMEKFILEDYETLKQKRMWEVHAALVRACEEAGVIAPSYKAFTKEVQRRTGYEQTKKRQGRRAAYQHESFYWELAITTPRHGDRPFEIGHIDHTELDVELVCSDTGRNLGRPWATFLVDAYSRRLLAVYLTFDSPSYRSCLMVLRICVKRHGRLPQIVVVDNGAEFHSVYFETLLATFECTKKQRPPAKARFGSVCERLFGTSNTQFVHNLLGNTQITRNVRQVTKSVNPKNLAVWTLGLLYEYLCAWAYEVYDTDEHPALFQSPRDAFAAGMAIGGSRVHRMIPYDENFQILTLPTTSEGKAKVQVGRGIKINYIYYWSNSFRDPQIENTSVQIRYDPFNIGIAYAFVRGQWVQCISQYYAELQGRSEKELKLASIELRKHYSNHTQQSKVSAKNLAEFLASVEAQEALLEQRSYDAEVKEVFRVIEGGKATTSRNEEPKLIQVTFANTDFQADEDEAIVPETLVVYEEFGGSGGGGSGGGGSGGPKKKRKVYPYDVPDYA

SEQ ID NO: 98 - protein, 414 aa, Artificial Sequence
  Synthetic
MTKSTGFPLELLTRPATERLAYFENYTVAHPRLKEVYEILMRTIAEPAGASFIFVYGASGVGKTTLRLRVEQKLTELALPKLESDRARVPVVGIEAIAPESRYFNWKEYYTRALITLEEPLIDHKFDYGVRGISRDNFGKINVESKVVAPALRRALENALIHRHPDVFFVDEAQHFGKVASGYKLQDQLDCLKSLANMTGILHCLLGTYELLTFRNLSGQLSRRSVDIHFRRYCADSPEDVQAFKSVLLTFQQHLPLAETPNLVDHWEYFYERTLGCIGTLKDWLKRVLSDALDREATTITLKDLQKRALSVAQCQKMFKEIQEGERQLSETEADVQNLRSALGLGAKPIVLPEETPKTTRPPGKVGKRKPKRDPIGVQQDVSGGSGGGGSGGGGSGGPKKKRKVYPYDVPDYA

SEQ ID NO: 99 - DNA, 3815 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcaatggctggtactataattcccctggttgctgaagcggtaaaagtgcgtcaactagttttgtgactttacccataactttgccgttactggaaccataactttgccgtcaacaacactacaaccctcattcttgcgtcagctaattttggatgaaattgacagtttggaaccataactttgccgctagtccaaattggaaggataagcttgccgtgagtccaatcaagcatagaaagcaggaaaacgccgaatttttttagaatggagcagaagcgtcgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaacactcacgacgtgagtttggtttgtaagctgttatatgtaaaactagaacagcttatataactggaaccataaccttgccaccctttattggaagcataagcttgccgttgcggcaaagttatgggtaaagtcacatctaagctgtctctagcctttatttactgaatgggcagtaccagactcgaactgatgacaatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 100 - DNA, 3018 bp, Artificial Sequence
  Synthetic
aattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgtgagtcgtggctttatgccgttaggcgttgcaaccatctatgtatagctacgatggaatagtaaagggtgagtcgtggctttatgccgttaggcgttgctcaatttttt

SEQ ID NO: 101 - DNA, 3018 bp, Artificial Sequence
  Synthetic
aattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgtgagtcgtggctttatgccgttaggcgttgcagagaagtcatttaataaggccactgttaaacgtaggtgagtcgtggctttatgccgttaggcgttgctcaatttttt

SEQ ID NO: 102 - DNA, 37 bp, Escherichia coli
actcctgctgcacaacaactccactgcatgtgtcaga

SEQ ID NO: 103 - DNA, 37 bp, Escherichia coli
tctggcgcttgctaccttgctgcacccagaaatccat

SEQ ID NO: 104 - DNA, 37 bp, Escherichia coli
gcgttgctgccgctaatcattagcgttatagtgaatc

SEQ ID NO: 105 - DNA, 37 bp, Escherichia coli
gacttctcctgaacttaagtctcagacctatttggcc

SEQ ID NO: 106 - DNA, 37 bp, Escherichia coli
gaatattaatgttactatctggttgagactgttgata

SEQ ID NO: 107 - DNA, 37 bp, Escherichia coli
ttctaaatcgacatgggcagtcgttctgtacactttg

SEQ ID NO: 108 - DNA, 37 bp, Escherichia coli
taatcgcaataatatattggctgtctattcatcgtgt

SEQ ID NO: 109 - DNA, 37 bp, Escherichia coli
ggcacgctaacaacgttttgttaaaaaatgtgaatca

SEQ ID NO: 110 - DNA, 37 bp, Escherichia coli
ctccagaccccctacaaagaatgtggatattgtgata

SEQ ID NO: 111 - DNA, 37 bp, Escherichia coli
tgctcacagaaacggagcggcacctcttttaaccctt

SEQ ID NO: 112 - DNA, 344 bp, Artificial Sequence
  Synthetic
ggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgcttacgcagggcatccatttattactcaacc

SEQ ID NO: 113 - DNA, 20 bp, Artificial Sequence
  Synthetic
gtcccagtccgatctcttgc

SEQ ID NO: 114 - DNA, 22 bp, Artificial Sequence
  Synthetic
cctatttggccggtaatccctc

SEQ ID NO: 115 - DNA, 21 bp, Artificial Sequence
  Synthetic
tcgaatttgctctttttgcgc

SEQ ID NO: 116 - DNA, 26 bp, Artificial Sequence
  Synthetic
tgaaccgaattatatattgttgaacc

SEQ ID NO: 117 - DNA, 25 bp, Artificial Sequence
  Synthetic
tggtacaatcgtactcctttagagg

SEQ ID NO: 118 - DNA, 330 bp, Escherichia coli
gactcatctcaattatctcgccatgacctggctaagtcccagtccgatctcttgccgtcgcgttcacacctgaatgacttctcctgaacttaagtctcagacctatttggccggtaatccctctcgaatttgctctttttgcgccccgccttccatcaatattgtggaggaataattcggttcaacaatatataattcggttcaacaattaataaaacgattgaacaataacctctaaaggagtacgattgtaccaagtaaaagtgcgatcgcttccagaggttatttttatggttaggggtagaagggaatggcggggatccatggaga

SEQ ID NO: 119 - DNA, 330 bp, Escherichia coli
tctccatggatccccgccattcccttctacccctaaccataaaaataacctctggaagcgatcgcacttttacttggtacaatcgtactcctttagaggttattgttcaatcgttttattaattgttgaaccgaattatatattgttgaaccgaattattcctccacaatattgatggaaggcggggcgcaaaaagagcaaattcgagagggattaccggccaaataggtctgagacttaagttcaggagaagtcattcaggtgtgaacgcgacggcaagagatcggactgggacttagccaggtcatggcgagataattgagatgagtc

SEQ ID NO: 120 - DNA, 89 bp, Artificial Sequence
  Synthetic
ttcgaatttgctctttttgcgccccgccttcctgtggaggaataattcggttcaacaatatataattcggttcaagatcggaagagcgt

SEQ ID NO: 121 - DNA, 49 bp, Artificial Sequence
  Synthetic
tcggaatttgctctttttgcgcccctccttccatcaaccttaacagacc

SEQ ID NO: 122 - DNA, 49 bp, Artificial Sequence
  Synthetic
tccgaatttgctctttttgcgccccgccttccatcaaccttaacagact

SEQ ID NO: 123 - DNA, 49 bp, Artificial Sequence
  Synthetic
tcggaatttgctctttttgcgccccgccttccatcaaccttttcggttc

SEQ ID NO: 124 - DNA, 50 bp, Artificial Sequence
  Synthetic
tcggaatttgctctttttgcgccccgccttccatcaaccggaggaataat

SEQ ID NO: 125 - DNA, 50 bp, Artificial Sequence
  Synthetic
ttcgaatttgctctttttgcgccccgccttccatctgtggaggaataatt

SEQ ID NO: 126 - DNA, 50 bp, Artificial Sequence
  Synthetic
ttcgaatttgctctttttgcgccccgccttccatgtggaggaataattcg

SEQ ID NO: 127 - DNA, 50 bp, Artificial Sequence
  Synthetic
tcggaatttgctctttttgcgccccgccttcctgtggaggaataattcgg

SEQ ID NO: 128 - DNA, 50 bp, Artificial Sequence
  Synthetic
ttcgaatttgctctttttgcgccccgccttcctgtggaggaataattcgg

SEQ ID NO: 129 - DNA, 50 bp, Artificial Sequence; identical to SEQ ID NO: 127
  Synthetic

SEQ ID NO: 130 - DNA, 50 bp, Artificial Sequence; identical to SEQ ID NO: 128
  Synthetic

SEQ ID NO: 131 - DNA, 50 bp, Artificial Sequence; identical to SEQ ID NO: 128
  Synthetic

SEQ ID NO: 132 - DNA, 50 bp, Artificial Sequence; identical to SEQ ID NO: 128
  Synthetic

SEQ ID NO: 133 - DNA, 50 bp, Artificial Sequence; identical to SEQ ID NO: 128
  Synthetic

SEQ ID NO: 134 - DNA, 50 bp, Artificial Sequence
  Synthetic
tccgaatttgctctttttgcgccccgccttcctgtggaggaataattcgg

SEQ ID NO: 135 - DNA, 50 bp, Artificial Sequence; identical to SEQ ID NO: 127
  Synthetic

SEQ ID NO: 136 - DNA, 50 bp, Artificial Sequence; identical to SEQ ID NO: 134
  Synthetic

SEQ ID NO: 137 - DNA, 50 bp, Artificial Sequence; identical to SEQ ID NO: 127
  Synthetic

SEQ ID NO: 138 - DNA, 50 bp, Artificial Sequence; identical to SEQ ID NO: 134
  Synthetic

SEQ ID NO: 139 - DNA, 50 bp, Artificial Sequence; identical to SEQ ID NO: 127
  Synthetic

SEQ ID NO: 140 - DNA, 50 bp, Artificial Sequence; identical to SEQ ID NO: 128
  Synthetic

SEQ ID NO: 141 - DNA, 50 bp, Artificial Sequence; identical to SEQ ID NO: 134
  Synthetic

SEQ ID NO: 142 - DNA, 50 bp, Artificial Sequence
  Synthetic
tcggaatttgctctttttgcgccccgctgtggaggaataattcggttcaa

SEQ ID NO: 143 - DNA, 50 bp, Artificial Sequence
  Synthetic
tccgaatttgctctttttgcgcccctgtggaggaataattcggttcaaca

SEQ ID NO: 144 - DNA, 50 bp, Artificial Sequence
  Synthetic
tcggaatttgctctttttgcgcccctgtggaggaataattcggttcaaca

SEQ ID NO: 145 - DNA, 50 bp, Artificial Sequence
  Synthetic
tccgaatttgctctttttgcgccccgctggaggaataattcggttcaaca

SEQ ID NO: 146 - DNA, 50 bp, Artificial Sequence
  Synthetic
ttcgaatttgctctttttgcgcccctgtggaggaataattcggttcaaca

SEQ ID NO: 147 - DNA, 50 bp, Artificial Sequence; identical to SEQ ID NO: 144
  Synthetic

SEQ ID NO: 148 - DNA, 19 bp, Artificial Sequence
  Synthetic
aataatacggttgaacaat

SEQ ID NO: 149 - DNA, 19 bp, Artificial Sequence
  Synthetic
aataattcggttcaacaat

SEQ ID NO: 150 - DNA, 19 bp, Artificial Sequence
  Synthetic
tataattcggttcaacaat

SEQ ID NO: 151 - DNA, 19 bp, Artificial Sequence
  Synthetic
aataaaacgattgaacaat

SEQ ID NO: 152 - DNA, 19 bp, Artificial Sequence
  Synthetic
aggagtacgattgtaccaa

SEQ ID NO: 153 - DNA, 19 bp, Artificial Sequence
  Synthetic
aataaaacggttaaacaat

SEQ ID NO: 154 - DNA, 19 bp, Artificial Sequence
  Synthetic
aataaaacgattcaacaat

SEQ ID NO: 155 - DNA, 19 bp, Artificial Sequence
  Synthetic
aagaattcggttaaacaat

SEQ ID NO: 156 - DNA, 26 bp, Artificial Sequence
  Synthetic
tgaaccgaattatatattgttcaacc

SEQ ID NO: 157 - DNA, 23 bp, Artificial Sequence
  Synthetic
tggcaaaatcggttacggttgag

SEQ ID NO: 158 - DNA, 105 bp, Artificial Sequence
  Synthetic
ttattaattgttgaaccgaattatatattgttgaaccgaattattcctccacacccgcttacgcagggcatccatttattactcaaccgtaaccgattttgccag

SEQ ID NO: 159 - DNA, 105 bp, Artificial Sequence
  Synthetic
ctggcaaaatcggttacggttgagtaataaatggatgccctgcgtaagcgggtgtggaggaataattcggttcaacaatatataattcggttcaacaattaataa

SEQ ID NO: 160 - DNA, 105 bp, Artificial Sequence; identical to SEQ ID NO: 158
  Synthetic

SEQ ID NO: 161 - DNA, 104 bp, Artificial Sequence
  Synthetic
ttattattgttgaaccgaattatatattgttgaaccgaattattcctccacacccgcttacgcagggcatccatttattactcaaccgtaaccgattttgccag

SEQ ID NO: 162 - DNA, 95 bp, Artificial Sequence
  Synthetic
ttgaaccgaattatatattgttgaaccgaattattcctccacacccgcttacgcagggcatccatttattactcaaccgtaaccgattttgccag

SEQ ID NO: 163 - DNA, 37 bp, Artificial Sequence; identical to SEQ ID NO: 66
  Synthetic

SEQ ID NO: 164 - DNA, 45 bp, Artificial Sequence
  Synthetic
atgccgtcgttacttcctaaagccagcattgtaccatcggggcta

SEQ ID NO: 165 - DNA, 36 bp, Artificial Sequence; identical to SEQ ID NO: 75
  Synthetic

SEQ ID NO: 166 - DNA, 16 bp, Artificial Sequence
  Synthetic
aggcattacacaatac

SEQ ID NO: 167 - DNA, 47 bp, Artificial Sequence
  Synthetic
atgagccagatcaccatccaggccagactgatcagcttcgagagcaa

SEQ ID NO: 168 - DNA, 21 bp, Artificial Sequence
  Synthetic
cgcaaatgggcggtaggcgtg

SEQ ID NO: 169 - DNA, 46 bp, Artificial Sequence
  Synthetic
ctaccggactcagatcgccaccatgatcgcccctctgatcctgtac

SEQ ID NO: 170 - DNA, 21 bp, Artificial Sequence
  Synthetic
catggtggcgatctgagtccg

SEQ ID NO: 171 - DNA, 19 bp, Artificial Sequence
  Synthetic
ggaggatctggcggaggcg

SEQ ID NO: 172 - DNA, 20 bp, Artificial Sequence
  Synthetic
tgactagcataaccccttgg

SEQ ID NO: 173 - DNA, 45 bp, Artificial Sequence
  Synthetic
cagagccgcctccgccagatcctccgatggggctctgcaccattg

SEQ ID NO: 174 - DNA, 47 bp, Artificial Sequence
  Synthetic
taggcagcctgcacctgaggagtgcgccccaaggggttatgctagtc

SEQ ID NO: 175 - DNA, 1000 bp, Artificial Sequence
  Synthetic
ttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcgccaccatgatcgcccctctgatcctgtacctggacgtgcccttcaccaccttcagagagtctcacgccagagagatgggcaagacataccccgttcctccaccagccacagtgtacggcatgctgctgtctctcgtgggcgagacaaacgtgtacagacactgcggcgtggaactggccattgccatgctgagcagccccaagaagtcccggatcctgcggcagatgcggagattcaagaacgccgacttctctcaccccgagaacgtgatcccttgctaccaagagatcctgagcaacctgaagtgcctgatctgggtccgaagcgacgaggaaaagatccagcctagcctgagagagcggatccagctggcctttgatcaccctgaactcgtcagaagattcggctgcctgttcctgggcgagagcgaccagctgatcaagaccatcaagctggccagagaggactacctggaaggcgttagacagtgggccatccgggacaacagaggcagactgacactgccctattgggtcgaccacgtgggcagcagaaatacccggttcctgagataccggatcgaggaaatggaccggctgagccctcctgacctggcttggacaatggtgcagagccccatcggaggatctggcggaggcggctctggcggcggtggaagcggaggacctaagaagaagagaaaagtctaccccgacgacgtgccagactacgcctgactagcataaccccttggggcgcactcctcaggtgcaggctgcctatcagaaggtggtggctggtgtggccaatgccctggctcacaaataccactgagatctttttccc

SEQ ID NO: 176 - DNA, 1000 bp, Artificial Sequence
  Synthetic
gggaaaaagatctcagtggtatttgtgagccagggcattggccacaccagccaccaccttctgataggcagcctgcacctgaggagtgcgccccaaggggttatgctagtcaggcgtagtctggcacgtcgtaggggtagacttttctcttcttcttaggtcctccgcttccaccgccgccagagccgcctccgccagatcctccgatggggctctgcaccattgtccaagccaggtcaggagggctcagccggtccatttcctcgatccggtatctcaggaaccgggtatttctgctgcccacgtggtcgacccaatagggcagtgtcagtctgcctctgttgtcccggatggcccactgtctaacgccttccaggtagtcctctctggccagcttgatggtcttgatcagctggtcgctctcgcccaggaacaggcagccgaatcttctgacgagttcagggtgatcaaaggccagctggatccgctctctcaggctaggctggatcttttcctcgtcgcttcggacccagatcaggcacttcaggttgctcaggatctcttggtagcaagggatcacgttctcggggtgagagaagtcggcgttcttgaatctccgcatctgccgcaggatccgggacttcttggggctgctcagcatggcaatggccagttccacgccgcagtgtctgtacacgtttgtctcgcccacgagagacagcagcatgccgtacactgtggctggtggaggaacggggtatgtcttgcccatctctctggcgtgagactctctgaaggtggtgaagggcacgtccaggtacaggatcagaggggcgatcatggtggcgatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaa

SEQ ID NO: 177 - DNA, 47 bp, Artificial Sequence; identical to SEQ ID NO: 167
  Synthetic

SEQ ID NO: 178 - protein, 243 aa, Artificial Sequence; identical to SEQ ID NO: 83
  Synthetic

SEQ ID NO: 179 - DNA, 47 bp, Artificial Sequence; identical to SEQ ID NO: 167
  Synthetic

SEQ ID NO: 180 - DNA, 42 bp, Artificial Sequence
  Synthetic
ccggactcagatcgccaccatgaccgccatcctgatccagag

SEQ ID NO: 181 - DNA, 21 bp, Artificial Sequence
  Synthetic
catggtggcgatctgagtccg

SEQ ID NO: 182 - DNA, 19 bp, Artificial Sequence
  Synthetic
ggaggatctggcggaggcg

SEQ ID NO: 183 - DNA, 51 bp, Artificial Sequence
  Synthetic
cagagccgcctccgccagatcctccggtttcctcttcctctttggatctgg

SEQ ID NO: 184 - DNA, 20 bp, Artificial Sequence
  Synthetic
tgactagcataaccccttgg

SEQ ID NO: 185 - DNA, 47 bp, Artificial Sequence; identical to SEQ ID NO: 167
  Synthetic

SEQ ID NO: 186 - DNA, 763 bp, Artificial Sequence
  Synthetic
ccggactcagatcgccaccatgaccgccatcctgatccagagcgaggaatacgtggacctgaccttcaagctgagaggcgcccctattcctctggacaacggctacctgacatacgccgctctgagcagaatctgccctcctctgcacgaactgaagtccatcggcattcaccctatcgccggcattcccaccagaaacaacctgctggaactgacagcccagagccggctgaagatcagaatctaccaccagcagatccctctgatctacccctacctggccggacaggcctttcacatcggccagaacttctaccagctggacatccccgactacaagcccctgatcagcagcgagagcgtgtacagcagactggtcatcatcaagggcttccaggacagcaccaacttcatcgaggccgtgcagcggcagatggacaatctgggaatccagggcaagatcgagctgctgaccagacaggatggcacccctcagagaaggcagctgaccatcaacaaagagggcaagcagttcaaagtgcgcggcttcggcgtgaagatctccgagctgaaccctgaggacagcctgacactgcaagagcaaggcatcggcggcaagcggaagatgatgtgcggcatctttgtgcccgccaccagatccaaagaggaagaggaaaccggaggatctggcggaggcggctctggcggcggtggaagcggaggacctaagaagaagagaaaagtctacccctacgacgtgcca

SEQ ID NO: 187 - DNA, 900 bp, Artificial Sequence
  Synthetic
cataatttttggcagagggaaaaagatctcagtggtatttgtgagccagggcattggccacaccagccaccaccttctgataggcagcctgcacctgaggagtgcgccccaaggggttatgctagtcaggcgtagtctggcacgtcgtaggggtagacttttctcttcttcttaggtcctccgcttccaccgccgccagagccgcctccgccagatcctccggtttcctcttcctctttggatctggtggcgggcacaaagatgccgcacatcatcttccgcttgccgccgatgccttgctcttgcagtgtcaggctgtcctcagggttcagctcggagatcttcacgccgaagccgcgcactttgaactgcttgccctctttgttgatggtcagctgccttctctgaggggtgccatcctgtctggtcagcagctcgatcttgccctggattcccagattgtccatctgccgctgcacggcctcgatgaagttggtgctgtcctggaagcccttgatgatgaccagtctgctgtacacgctctcgctgctgatcaggggcttgtagtcggggatgtccagctggtagaagttctggccgatgtgaaaggcctgtccggccaggtaggggtagatcagagggatctgctggtggtagattctgatcttcagccggctctgggctgtcagttccagcaggttgtttctggtgggaatgccggcgatagggtgaatgccgatggacttcagttcgtgcagaggagggcagattctgctcagagcggcgtatgtcaggtagccgttgtccagaggaataggggcgcctctcagcttgaaggtcaggtccacgtattcctcgctctggatcaggatggcggtcatggtggcgatctgagtccgg

SEQ ID NO: 188 - protein, 251 aa, Artificial Sequence; identical to SEQ ID NO: 84
  Synthetic

SEQ ID NO: 189 - DNA, 43 bp, Artificial Sequence
  Synthetic
atgagccagatcaccaggccagactgatcagcttcgagagcaa

SEQ ID NO: 190 - DNA, 48 bp, Artificial Sequence
  Synthetic
ctaccggactcagatcgccaccatgatgacccagaagaagaacgacag

SEQ ID NO: 191 - DNA, 21 bp, Artificial Sequence
  Synthetic
catggtggcgatctgagtccg

SEQ ID NO: 192 - skipped

SEQ ID NO: 193 - DNA, 19 bp, Artificial Sequence
  Synthetic
ggaggatctggcggaggcg

SEQ ID NO: 194 - DNA, 44 bp, Artificial Sequence
  Synthetic
cagagccgcctccgccagatcctcccttggattcctcgaggccc

SEQ ID NO: 195 - DNA, 18 bp, Artificial Sequence
  Synthetic
tgactagcataacccctt

SEQ ID NO: 196 - DNA, 17 bp, Artificial Sequence
  Synthetic
aaggggttatgctagtc

SEQ ID NO: 197 - DNA, 47 bp, Artificial Sequence; identical to SEQ ID NO: 167
  Synthetic

SEQ ID NO: 198 - DNA, 1121 bp, Artificial Sequence
  Synthetic
accgtcagatccgctagcgctaccggactcagatcgccaccatgatgacccagaagaagaacgacagcaacatccccaactactacctgtacggcaccgtgctgaccagatacggactggccagcctgaaccacgacatcagaagaggcaacaagaccatcctgcagaaaggctactggaacaacggcaagatccacagcttcgtgggcagcagcgccattagatgggccctgaggttctatctgcagaaacagggctacctggtcaacagagtgtgggacgaagaggaacacatcaacagactgaccagcgaggacttcgaccccgagaagttctacgacgacgacatcttcggcttcgccctgctggaaagcgccgagacagaggaagataccagcaccaccaagcggaagaagaagcagaccaagaccagcacacccaaccagagaatgggcgccctgggcatgaatatggccgtgtctctgaccccttacgatggcgccgtgaagctgggagccaagtctggcagagagaaggacagcaccagcctgcacttcaccgagtaccacgccaccagataccagtactacttcggcatcgacgccacacacctgaaggacttcagccggatcctgcctatgatcgacggcatcatgaacctgcctaaagtcggcggcagcagcaatatcttcaactaccctttctgccccgacagcctggtgttccagtggaccaatcacttcgccagctacatcagctactgcttcgagtactgcgaccccaagagcaaagaggccaagctgagccaagagttcatcgacgaggtggaatgcggccagatcgaccctagcaaactgtggatcggcggcaccatcgtgaaggacctgcagcagctggacaacttcgagagcagccctctgaacaaggcccacatctaccggaaccggaacgagatgatcgaggccctgaaaaccgtgatcaagcgcgacctgggcctcgaggaatccaagggaggatctggcggaggcggctctggcggcggtggaagcggaggacctaagaagaagagaaaagtctacccctacgacgtgccagactacgcctgactagcataacccctt

SEQ ID NO: 199 - DNA, 1121 bp, Artificial Sequence
  Synthetic
aaggggttatgctagtcaggcgtagtctggcacgtcgtaggggtagacttttctcttcttcttaggtcctccgcttccaccgccgccagagccgcctccgccagatcctcccttggattcctcgaggcccaggtcgcgcttgatcacggttttcagggcctcgatcatctcgttccggttccggtagatgtgggccttgttcagagggctgctctcgaagttgtccagctgctgcaggtccttcacgatggtgccgccgatccacagtttgctagggtcgatctggccgcattccacctcgtcgatgaactcttggctcagcttggcctctttgctcttggggtcgcagtactcgaagcagtagctgatgtagctggcgaagtgattggtccactggaacaccaggctgtcggggcagaaagggtagttgaagatattgctgctgccgccgactttaggcaggttcatgatgccgtcgatcataggcaggatccggctgaagtccttcaggtgtgtggcgtcgatgccgaagtagtactggtatctggtggcgtggtactcggtgaagtgcaggctggtgctgtccttctctctgccagacttggctcccagcttcacggcgccatcgtaaggggtcagagacacggccatattcatgcccagggcgcccattctctggttgggtgtgctggtcttggtctgcttcttcttccgcttggtggtgctggtatcttcctctgtctcggcgctttccagcagggcgaagccgaagatgtcgtcgtcgtagaacttctcggggtcgaagtcctcgctggtcagtctgttgatgtgttcctcttcgtcccacactctgttgaccaggtagccctgtttctgcagatagaacctcagggcccatctaatggcgctgctgcccacgaagctgtggatcttgccgttgttccagtagcctttctgcaggatggtcttgttgcctcttctgatgtcgtggttcaggctggccagtccgtatctggtcagcacggtgccgtacaggtagtagttggggatgttgctgtcgttcttcttctgggtcatcatggtggcgatctgagtccggtagcgctagcggatctgacggt

SEQ ID NO: 200 - protein, 354 aa, Artificial Sequence; identical to SEQ ID NO: 85
  Synthetic

SEQ ID NO: 201 - DNA, 47 bp, Artificial Sequence; identical to SEQ ID NO: 167
  Synthetic

SEQ ID NO: 202 - DNA, 46 bp, Artificial Sequence
  Synthetic
ctaccggactcagatcgccaccatgatcgtgtccacacagcccaag

SEQ ID NO: 203 - DNA, 21 bp, Artificial Sequence
  Synthetic
catggtggcgatctgagtccg

SEQ ID NO: 204 - DNA, 19 bp, Artificial Sequence
  Synthetic
ggaggatctggcggaggcg

SEQ ID NO: 205 - DNA, 19 bp, Artificial Sequence
  Synthetic
ggaggatctggcggaggcg

SEQ ID NO: 206 - DNA, 47 bp, Artificial Sequence
  Synthetic
cagagccgcctccgccagatcctccctcttcctcgctgtcgtcgttc

SEQ ID NO: 207 - DNA, 20 bp, Artificial Sequence
  Synthetic
tgactagcataaccccttgg

SEQ ID NO: 208 - DNA, 23 bp, Artificial Sequence
  Synthetic
cgccccaaggggttatgctagtc

SEQ ID NO: 209 - DNA, 47 bp, Artificial Sequence; identical to SEQ ID NO: 167
  Synthetic

SEQ ID NO: 210 - DNA, 1726 bp, Artificial Sequence
  Synthetic
ccgctagcgctaccggactcagatcgccaccatgatcgtgtccacacagcccaagatcagcctgtctctgcacgccgccgataccaccatcatgcacagagtgggcatgaccggcctgtacatgaccctgaagcggctggaaaagcagtaccctctgagcagacagagaggcggccacatcagctggtttctgaccgccgacaccatcgagctgttttgggagggcagcgactttatcgccctgagctggctgatcaacgagagctttcagctggacgacaccggcctgatccacctcgttggcctggacaacgaccggatcgacctgagacagaagatccacatccacgagggaatctgcggcgtgttcctgcggctgaacaagttttaccaggccggcgagatcatcaacaccgagctgagattcgaagagaagcaggtcgagtaccagtacaagagcctgacttggtacgcccaccagaccttcgccgagaagctgtgcgaagccgatacacagcagctgcggcacgactacatccagatcaccagctggctgtacctcggcggaattgtgcggcatgccagaacacagaacaccaccaagctggaagagaaacccgagtacgccctggctctgctgtttgtgcctgtcgtgtgccactactgcctgctgcacatccctagcgaggacctgaaagagcggaagccccacagatacctggtggtcatccccgagatcaaggacttcgaggatgccagccagagaagatggcggctgcagcagctggaaacaaagcagttccacgtgtccagcctgggcgaagccggcctgctgtactacagcctggatgacattcagcccgaggtggcctactaccaggcctgtcaagtgtggctgtacgaaaagaccaacaaggccagccggcagcggaccctgatgagcatcgaggaaatcaagatcgacaagaacatcctcatcacctaccagcaggtccagaagtactttaagaccaactaccagatcattaagtacaagcagatcttcatcaaagtgaaccccattcggagcctgatcgccgacaatctggtcaagggaatccactggtggtccaatttctgggagaagctggtcatcgaggacagcaaagagtacctgttcaaccagctgttcagcaaccgcgagggcttcatcatcatggccgagaacagcgaagaggacaagcagtatctgatctttatcaaggtgttccagcaggccatgaagggcaacttcgccaagatctacgccaagaccgaggaaggcaaggaccctcctatcaagaaaaaggtggaacggctgcgggccgagctgaactactgttacgatgagctgagcttcaaagaatacctgagcgacttcctcgtcagaggcggactgaacaaatacttcaacgagcaccaagaggaaatcgccctgctgatcaagaagtccccttggcaagagatcagaatctggtccctgctggccattgccagctacaagcccaaggacaagctgaccaaccgggacgatagcagcctgagcaacaaccagaaactggaagaagtgaacgacgacagcgaggaagagggaggatctggcggaggcggctctggcggcggtggaagcggaggacctaagaagaagagaaaagtctacccctacgacgtgccagactacgcctgactagcataaccccttggggcg

SEQ ID NO: 211 - DNA, 1726 bp, Artificial Sequence
  Synthetic
cgccccaaggggttatgctagtcaggcgtagtctggcacgtcgtaggggtagacttttctcttcttcttaggtcctccgcttccaccgccgccagagccgcctccgccagatcctccctcttcctcgctgtcgtcgttcacttcttccagtttctggttgttgctcaggctgctatcgtcccggttggtcagcttgtccttgggcttgtagctggcaatggccagcagggaccagattctgatctcttgccaaggggacttcttgatcagcagggcgatttcctcttggtgctcgttgaagtatttgttcagtccgcctctgacgaggaagtcgctcaggtattctttgaagctcagctcatcgtaacagtagttcagctcggcccgcagccgttccacctttttcttgataggagggtccttgccttcctcggtcttggcgtagatcttggcgaagttgcccttcatggcctgctggaacaccttgataaagatcagatactgcttgtcctcttcgctgttctcggccatgatgatgaagccctcgcggttgctgaacagctggttgaacaggtactctttgctgtcctcgatgaccagcttctcccagaaattggaccaccagtggattcccttgaccagattgtcggcgatcaggctccgaatggggttcactttgatgaagatctgcttgtacttaatgatctggtagttggtcttaaagtacttctggacctgctggtaggtgatgaggatgttcttgtcgatcttgatttcctcgatgctcatcagggtccgctgccggctggccttgttggtcttttcgtacagccacacttgacaggcctggtagtaggccacctcgggctgaatgtcatccaggctgtagtacagcaggccggcttcgcccaggctggacacgtggaactgctttgtttccagctgctgcagccgccatcttctctggctggcatcctcgaagtccttgatctcggggatgaccaccaggtatctgtggggcttccgctctttcaggtcctcgctagggatgtgcagcaggcagtagtggcacacgacaggcacaaacagcagagccagggcgtactcgggtttctcttccagcttggtggtgttctgtgttctggcatgccgcacaattccgccgaggtacagccagctggtgatctggatgtagtcgtgccgcagctgctgtgtatcggcttcgcacagcttctcggcgaaggtctggtgggcgtaccaagtcaggctcttgtactggtactcgacctgcttctcttcgaatctcagctcggtgttgatgatctcgccggcctggtaaaacttgttcagccgcaggaacacgccgcagattccctcgtggatgtggatcttctgtctcaggtcgatccggtcgttgtccaggccaacgaggtggatcaggccggtgtcgtccagctgaaagctctcgttgatcagccagctcagggcgataaagtcgctgccctcccaaaacagctcgatggtgtcggcggtcagaaaccagctgatgtggccgcctctctgtctgctcagagggtactgcttttccagccgcttcagggtcatgtacaggccggtcatgcccactctgtgcatgatggtggtatcggcggcgtgcagagacaggctgatcttgggctgtgtggacacgatcatggtggcgatctgagtccggtagcgctagcgg

SEQ ID NO: 212 - protein, 198 aa, Artificial Sequence
  Synthetic
MPHNSAEVQPAGDLDVVVPQLLCIGFAQLLGEGLVGVPSQALVLVLDLLLFESQLGVDDLAGLVKLVQPQEHAADSLVDVDLLSQVDPVVVQANEVDQAGVVQLKALVDQPAQGDKVAALPKQLDGVGGQKPADVAASLSAQRVLLFQPLQGHVQAGHAHSVHDGGIGGVQRQADLGLCGHDHGGDLSPVALADLTVH

SEQ ID NO: 213 - protein, 557 aa, Artificial Sequence; identical to SEQ ID NO: 86
  Synthetic

SEQ ID NO: 214 - DNA, 47 bp, Artificial Sequence; identical to SEQ ID NO: 167
  Synthetic

SEQ ID NO: 215 - DNA, 21 bp, Artificial Sequence
  Synthetic
cgcaaatgggcggtaggcgtg

SEQ ID NO: 216 - DNA, 49 bp, Artificial Sequence
  Synthetic
ccgctagcgctaccggactcagatcgccaccatgtacccctacgacgtg

SEQ ID NO: 217 - DNA, 46 bp, Artificial Sequence
  Synthetic
atctggcggaggtggaagcggaggaatcgcccctctgatcctgtac

SEQ ID NO: 218 - DNA, 20 bp, Artificial Sequence
  Synthetic
tcctccgcttccacctccgc

SEQ ID NO: 219 - DNA, 20 bp, Artificial Sequence
  Synthetic
ctagcataaccccttggggc

SEQ ID NO: 220 - DNA, 20 bp, Artificial Sequence
  Synthetic
tgactagcataaccccttgg

SEQ ID NO: 221 - DNA, 47 bp, Artificial Sequence; identical to SEQ ID NO: 167
  Synthetic

SEQ ID NO: 222 - DNA, 44 bp, Artificial Sequence
  Synthetic
agtgcgccccaaggggttatgctagtcagatggggctctgcacc

SEQ ID NO: 223 - DNA, 10 bp, Artificial Sequence
  Synthetic
gggaaaaaga

SEQ ID NO: 224 - DNA, 1000 bp, Artificial Sequence
  Synthetic
ttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcgccaccatgtacccctacgacgtgccagactacgcccctaagaagaagagaaaagtcggcggctctggcggcggaggatctggcggaggtggaagcggaggaatcgcccctctgatcctgtacctggacgtgcccttcaccaccttcagagagtctcacgccagagagatgggcaagacataccccgttcctccaccagccacagtgtacggcatgctgctgtctctcgtgggcgagacaaacgtgtacagacactgcggcgtggaactggccattgccatgctgagcagccccaagaagtcccggatcctgcggcagatgcggagattcaagaacgccgacttctctcaccccgagaacgtgatcccttgctaccaagagatcctgagcaacctgaagtgcctgatctgggtccgaagcgacgaggaaaagatccagcctagcctgagagagcggatccagctggcctttgatcaccctgaactcgtcagaagattcggctgcctgttcctgggcgagagcgaccagctgatcaagaccatcaagctggccagagaggactacctggaaggcgttagacagtgggccatccgggacaacagaggcagactgacactgccctattgggtcgaccacgtgggcagcagaaatacccggttcctgagataccggatcgaggaaatggaccggctgagccctcctgacctggcttggacaatggtgcagagccccatctgactagcataaccccttggggcgcactcctcaggtgcaggctgcctatcagaaggtggtggctggtgtggccaatgccctggctcacaaataccactgagatctttttccc

SEQ ID NO: 225 - DNA, 1000 bp, Artificial Sequence
  Synthetic
gggaaaaagatctcagtggtatttgtgagccagggcattggccacaccagccaccaccttctgataggcagcctgcacctgaggagtgcgccccaaggggttatgctagtcagatggggctctgcaccattgtccaagccaggtcaggagggctcagccggtccatttcctcgatccggtatctcaggaaccgggtatttctgctgcccacgtggtcgacccaatagggcagtgtcagtctgcctctgttgtcccggatggcccactgtctaacgccttccaggtagtcctctctggccagcttgatggtcttgatcagctggtcgctctcgcccaggaacaggcagccgaatcttctgacgagttcagggtgatcaaaggccagctggatccgctctctcaggctaggctggatcttttcctcgtcgcttcggacccagatcaggcacttcaggttgctcaggatctcttggtagcaagggatcacgttctcggggtgagagaagtcggcgttcttgaatctccgcatctgccgcaggatccgggacttcttggggctgctcagcatggcaatggccagttccacgccgcagtgtctgtacacgtttgtctcgcccacgagagacagcagcatgccgtacactgtggctggtggaggaacggggtatgtcttgcccatctctctggcgtgagactctctgaaggtggtgaagggcacgtccaggtacaggatcagaggggcgattcctccgcttccacctccgccagatcctccgccgccagagccgccgacttttctcttcttcttaggggcgtagtctggcacgtcgtaggggtacatggtggcgatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaa

SEQ ID NO: 226 - protein, 243 aa, Artificial Sequence; identical to SEQ ID NO: 87
  Synthetic

SEQ ID NO: 227 - DNA, 47 bp, Artificial Sequence; identical to SEQ ID NO: 167
  Synthetic

SEQ ID NO: 228 - DNA, 21 bp, Artificial Sequence
  Synthetic
cgcaaatgggcggtaggcgtg

SEQ ID NO: 229 - DNA, 49 bp, Artificial Sequence; identical to SEQ ID NO: 216
  Synthetic

SEQ ID NO: 230 - DNA, 45 bp, Artificial Sequence
  Synthetic
atctggcggaggtggaagcggaggaaccgccatcctgatccagag

SEQ ID NO: 231 - DNA, 20 bp, Artificial Sequence
  Synthetic
tcctccgcttccacctccgc

SEQ ID NO: 232 - DNA, 20 bp, Artificial Sequence
  Synthetic
ctagcataaccccttggggc

SEQ ID NO: 233 - DNA, 20 bp, Artificial Sequence
  Synthetic
tgactagcataaccccttgg

SEQ ID NO: 234 - DNA, 47 bp, Artificial Sequence; identical to SEQ ID NO: 167
  Synthetic

SEQ ID NO: 235 - DNA, 51 bp, Artificial Sequence
  Synthetic
agtgcgccccaaggggttatgctagtcaggtttcctcttcctctttggatc

SEQ ID NO: 236 - DNA, 1000 bp, Artificial Sequence
  Synthetic
ttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcgccaccatgtacccctacgacgtgccagactacgcccctaagaagaagagaaaagtcggcggctctggcggcggaggatctggcggaggtggaagcggaggaaccgccatcctgatccagagcgaggaatacgtggacctgaccttcaagctgagaggcgcccctattcctctggacaacggctacctgacatacgccgctctgagcagaatctgccctcctctgcacgaactgaagtccatcggcattcaccctatcgccggcattcccaccagaaacaacctgctggaactgacagcccagagccggctgaagatcagaatctaccaccagcagatccctctgatctacccctacctggccggacaggcctttcacatcggccagaacttctaccagctggacatccccgactacaagcccctgatcagcagcgagagcgtgtacagcagactggtcatcatcaagggcttccaggacagcaccaacttcatcgaggccgtgcagcggcagatggacaatctgggaatccagggcaagatcgagctgctgaccagacaggatggcacccctcagagaaggcagctgaccatcaacaaagagggcaagcagttcaaagtgcgcggcttcggcgtgaagatctccgagctgaaccctgaggacagcctgacactgcaagagcaaggcatcggcggcaagcggaagatgatgtgcggcatctttgtgcccgccaccagatccaaagaggaagaggaaacctgactagcataaccccttggggcgcactcctcaggtgcaggctgcctatcagaaggtggtggctggtgtggccaatgccctggctcac

SEQ ID NO: 237 - DNA, 1000 bp, Artificial Sequence
  Synthetic
gtgagccagggcattggccacaccagccaccaccttctgataggcagcctgcacctgaggagtgcgccccaaggggttatgctagtcaggtttcctcttcctctttggatctggtggcgggcacaaagatgccgcacatcatcttccgcttgccgccgatgccttgctcttgcagtgtcaggctgtcctcagggttcagctcggagatcttcacgccgaagccgcgcactttgaactgcttgccctctttgttgatggtcagctgccttctctgaggggtgccatcctgtctggtcagcagctcgatcttgccctggattcccagattgtccatctgccgctgcacggcctcgatgaagttggtgctgtcctggaagcccttgatgatgaccagtctgctgtacacgctctcgctgctgatcaggggcttgtagtcggggatgtccagctggtagaagttctggccgatgtgaaaggcctgtccggccaggtaggggtagatcagagggatctgctggtggtagattctgatcttcagccggctctgggctgtcagttccagcaggttgtttctggtgggaatgccggcgatagggtgaatgccgatggacttcagttcgtgcagaggagggcagattctgctcagagcggcgtatgtcaggtagccgttgtccagaggaataggggcgcctctcagcttgaaggtcaggtccacgtattcctcgctctggatcaggatggcggttcctccgcttccacctccgccagatcctccgccgccagagccgccgacttttctcttcttcttaggggcgtagtctggcacgtcgtaggggtacatggtggcgatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaa

SEQ ID NO: 238 - protein, 251 aa, Artificial Sequence; identical to SEQ ID NO: 88
  Synthetic

SEQ ID NO: 239 - DNA, 48 bp, Artificial Sequence
  Synthetic
atgagccagatcaccatcccaggccagactgatcagcttcgagagcaa

SEQ ID NO: 240 - DNA, 21 bp, Artificial Sequence
  Synthetic
cgcaaatgggcggtaggcgtg

SEQ ID NO: 241 - DNA, 49 bp, Artificial Sequence; identical to SEQ ID NO: 216
  Synthetic

SEQ ID NO: 242 - DNA, 48 bp, Artificial Sequence
  Synthetic
atctggcggaggtggaagcggaggaatgacccagaagaagaacgacag

SEQ ID NO: 243 - DNA, 20 bp, Artificial Sequence
  Synthetic
tcctccgcttccacctccgc

SEQ ID NO: 244 - DNA, 20 bp, Artificial Sequence
  Synthetic
ctagcataaccccttggggc

SEQ ID NO: 245 - DNA, 20 bp, Artificial Sequence
  Synthetic
tgactagcataaccccttgg

SEQ ID NO: 246 - DNA, 47 bp, Artificial Sequence; identical to SEQ ID NO: 167
  Synthetic

SEQ ID NO: 247 - DNA, 1320 bp, Artificial Sequence
  Synthetic
acaactccgccccattgacgcaaatgggcggtaggcgtgtacggtgggaggtctatataagcagagctggtttagtgaaccgtcagatccgctagcgctaccggactcagatcgccaccatgtacccctacgacgtgccagactacgcccctaagaagaagagaaaagtcggcggctctggcggcggaggatctggcggaggtggaagcggaggaatgacccagaagaagaacgacagcaacatccccaactactacctgtacggcaccgtgctgaccagatacggactggccagcctgaaccacgacatcagaagaggcaacaagaccatcctgcagaaaggctactggaacaacggcaagatccacagcttcgtgggcagcagcgccattagatgggccctgaggttctatctgcagaaacagggctacctggtcaacagagtgtgggacgaagaggaacacatcaacagactgaccagcgaggacttcgaccccgagaagttctacgacgacgacatcttcggcttcgccctgctggaaagcgccgagacagaggaagataccagcaccaccaagcggaagaagaagcagaccaagaccagcacacccaaccagagaatgggcgccctgggcatgaatatggccgtgtctctgaccccttacgatggcgccgtgaagctgggagccaagtctggcagagagaaggacagcaccagcctgcacttcaccgagtaccacgccaccagataccagtactacttcggcatcgacgccacacacctgaaggacttcagccggatcctgcctatgatcgacggcatcatgaacctgcctaaagtcggcggcagcagcaatatcttcaactaccctttctgccccgacagcctggtgttccagtggaccaatcacttcgccagctacatcagctactgcttcgagtactgcgaccccaagagcaaagaggccaagctgagccaagagttcatcgacgaggtggaatgcggccagatcgaccctagcaaactgtggatcggcggcaccatcgtgaaggacctgcagcagctggacaacttcgagagcagccctctgaacaaggcccacatctaccggaaccggaacgagatgatcgaggccctgaaaaccgtgatcaagcgcgacctgggcctcgaggaatccaagtaactagcataaccccttggggcgcactcctcaggtgcaggctgcctatcagaaggtggtggctggtgtggccaatgccctggctcacaaataccactgagatctttttccctctgccaaaaattatggggacatcatg

SEQ ID NO: 248 - DNA, 1320 bp, Artificial Sequence
  Synthetic
catgatgtccccataatttttggcagagggaaaaagatctcagtggtatttgtgagccagggcattggccacaccagccaccaccttctgataggcagcctgcacctgaggagtgcgccccaaggggttatgctagttacttggattcctcgaggcccaggtcgcgcttgatcacggttttcagggcctcgatcatctcgttccggttccggtagatgtgggccttgttcagagggctgctctcgaagttgtccagctgctgcaggtccttcacgatggtgccgccgatccacagtttgctagggtcgatctggccgcattccacctcgtcgatgaactcttggctcagcttggcctctttgctcttggggtcgcagtactcgaagcagtagctgatgtagctggcgaagtgattggtccactggaacaccaggctgtcggggcagaaagggtagttgaagatattgctgctgccgccgactttaggcaggttcatgatgccgtcgatcataggcaggatccggctgaagtccttcaggtgtgtggcgtcgatgccgaagtagtactggtatctggtggcgtggtactcggtgaagtgcaggctggtgctgtccttctctctgccagacttggctcccagcttcacggcgccatcgtaaggggtcagagacacggccatattcatgcccagggcgcccattctctggttgggtgtgctggtcttggtctgcttcttcttccgcttggtggtgctggtatcttcctctgtctcggcgctttccagcagggcgaagccgaagatgtcgtcgtcgtagaacttctcggggtcgaagtcctcgctggtcagtctgttgatgtgttcctcttcgtcccacactctgttgaccaggtagccctgtttctgcagatagaacctcagggcccatctaatggcgctgctgcccacgaagctgtggatcttgccgttgttccagtagcctttctgcaggatggtcttgttgcctcttctgatgtcgtggttcaggctggccagtccgtatctggtcagcacggtgccgtacaggtagtagttggggatgttgctgtcgttcttcttctgggtcattcctccgcttccacctccgccagatcctccgccgccagagccgccgacttttctcttcttcttaggggcgtagtctggcacgtcgtaggggtacatggtggcgatctgagtccggtagcgctagcggatctgacggttcactaaaccagctctgcttatatagacctcccaccgtacacgcctaccgcccatttgcgtcaatggggcggagttgt

SEQ ID NO: 249 - protein, 354 aa, Artificial Sequence; identical to SEQ ID NO: 89
  Synthetic

SEQ ID NO: 250 - DNA, 47 bp, Artificial Sequence; identical to SEQ ID NO: 167
  Synthetic

SEQ ID NO: 251 - DNA, 40 bp, Artificial Sequence
  Synthetic
ctaccggactcagatcgccaccatgtacccctacgacgtg

SEQ ID NO: 252 - DNA, 46 bp, Artificial Sequence
  Synthetic
atctggcggaggtggaagcggaggaatcgtgtccacacagcccaag

SEQ ID NO: 253 - DNA, 20 bp, Artificial Sequence
  Synthetic
tcctccgcttccacctccgc

SEQ ID NO: 254 - DNA, 18 bp, Artificial Sequence
  Synthetic
ctagcataaccccttggg

SEQ ID NO: 255 - DNA, 20 bp, Artificial Sequence
  Synthetic
tgactagcataaccccttgg

SEQ ID NO: 256 - DNA, 20 bp, Artificial Sequence
  Synthetic
atgagccagatcaccatcca

SEQ ID NO: 257 - DNA, 40 bp, Artificial Sequence
  Synthetic
cccaaggggttatgctagtcactcttcctcgctgtcgtcg

SEQ ID NO: 258 - DNA, 1714 bp, Artificial Sequence
  Synthetic
ctaccggactcagatcgccaccatgtacccctacgacgtgccagactacgcccctaagaagaagagaaaagtcggcggctctggcggcggaggatctggcggaggtggaagcggaggaatcgtgtccacacagcccaagatcagcctgtctctgcacgccgccgataccaccatcatgcacagagtgggcatgaccggcctgtacatgaccctgaagcggctggaaaagcagtaccctctgagcagacagagaggcggccacatcagctggtttctgaccgccgacaccatcgagctgttttgggagggcagcgactttatcgccctgagctggctgatcaacgagagctttcagctggacgacaccggcctgatccacctcgttggcctggacaacgaccggatcgacctgagacagaagatccacatccacgagggaatctgcggcgtgttcctgcggctgaacaagttttaccaggccggcgagatcatcaacaccgagctgagattcgaagagaagcaggtcgagtaccagtacaagagcctgacttggtacgcccaccagaccttcgccgagaagctgtgcgaagccgatacacagcagctgcggcacgactacatccagatcaccagctggctgtacctcggcggaattgtgcggcatgccagaacacagaacaccaccaagctggaagagaaacccgagtacgccctggctctgctgtttgtgcctgtcgtgtgccactactgcctgctgcacatccctagcgaggacctgaaagagcggaagccccacagatacctggtggtcatccccgagatcaaggacttcgaggatgccagccagagaagatggcggctgcagcagctggaaacaaagcagttccacgtgtccagcctgggcgaagccggcctgctgtactacagcctggatgacattcagcccgaggtggcctactaccaggcctgtcaagtgtggctgtacgaaaagaccaacaaggccagccggcagcggaccctgatgagcatcgaggaaatcaagatcgacaagaacatcctcatcacctaccagcaggtccagaagtactttaagaccaactaccagatcattaagtacaagcagatcttcatcaaagtgaaccccattcggagcctgatcgccgacaatctggtcaagggaatccactggtggtccaatttctgggagaagctggtcatcgaggacagcaaagagtacctgttcaaccagctgttcagcaaccgcgagggcttcatcatcatggccgagaacagcgaagaggacaagcagtatctgatctttatcaaggtgttccagcaggccatgaagggcaacttcgccaagatctacgccaagaccgaggaaggcaaggaccctcctatcaagaaaaaggtggaacggctgcgggccgagctgaactactgttacgatgagctgagcttcaaagaatacctgagcgacttcctcgtcagaggcggactgaacaaatacttcaacgagcaccaagaggaaatcgccctgctgatcaagaagtccccttggcaagagatcagaatctggtccctgctggccattgccagctacaagcccaaggacaagctgaccaaccgggacgatagcagcctgagcaacaaccagaaactggaagaagtgaacgacgacagcgaggaagagtgactagcataaccccttggg

SEQ ID NO: 259 - DNA, 1714 bp, Artificial Sequence
  Synthetic
cccaaggggttatgctagtcactcttcctcgctgtcgtcgttcacttcttccagtttctggttgttgctcaggctgctatcgtcccggttggtcagcttgtccttgggcttgtagctggcaatggccagcagggaccagattctgatctcttgccaaggggacttcttgatcagcagggcgatttcctcttggtgctcgttgaagtatttgttcagtccgcctctgacgaggaagtcgctcaggtattctttgaagctcagctcatcgtaacagtagttcagctcggcccgcagccgttccacctttttcttgataggagggtccttgccttcctcggtcttggcgtagatcttggcgaagttgcccttcatggcctgctggaacaccttgataaagatcagatactgcttgtcctcttcgctgttctcggccatgatgatgaagccctcgcggttgctgaacagctggttgaacaggtactctttgctgtcctcgatgaccagcttctcccagaaattggaccaccagtggattcccttgaccagattgtcggcgatcaggctccgaatggggttcactttgatgaagatctgcttgtacttaatgatctggtagttggtcttaaagtacttctggacctgctggtaggtgatgaggatgttcttgtcgatcttgatttcctcgatgctcatcagggtccgctgccggctggccttgttggtcttttcgtacagccacacttgacaggcctggtagtaggccacctcgggctgaatgtcatccaggctgtagtacagcaggccggcttcgcccaggctggacacgtggaactgctttgtttccagctgctgcagccgccatcttctctggctggcatcctcgaagtccttgatctcggggatgaccaccaggtatctgtggggcttccgctctttcaggtcctcgctagggatgtgcagcaggcagtagtggcacacgacaggcacaaacagcagagccagggcgtactcgggtttctcttccagcttggtggtgttctgtgttctggcatgccgcacaattccgccgaggtacagccagctggtgatctggatgtagtcgtgccgcagctgctgtgtatcggcttcgcacagcttctcggcgaaggtctggtgggcgtaccaagtcaggctcttgtactggtactcgacctgcttctcttcgaatctcagctcggtgttgatgatctcgccggcctggtaaaacttgttcagccgcaggaacacgccgcagattccctcgtggatgtggatcttctgtctcaggtcgatccggtcgttgtccaggccaacgaggtggatcaggccggtgtcgtccagctgaaagctctcgttgatcagccagctcagggcgataaagtcgctgccctcccaaaacagctcgatggtgtcggcggtcagaaaccagctgatgtggccgcctctctgtctgctcagagggtactgcttttccagccgcttcagggtcatgtacaggccggtcatgcccactctgtgcatgatggtggtatcggcggcgtgcagagacaggctgatcttgggctgtgtggacacgattcctccgcttccacctccgccagatcctccgccgccagagccgccgacttttctcttcttcttaggggcgtagtctggcacgtcgtaggggtacatggtggcgatctgagtccggtag

SEQ ID NO: 260 - protein, 557 aa, Artificial Sequence; identical to SEQ ID NO: 90
  Synthetic

SEQ ID NO: 261 - protein, 221 aa, Artificial Sequence
  Synthetic
MPHNSAEVQPAGDLDVVVPQLLCIGFAQLLGEGLVGVPSQALVLVLDLLLFESQLGVDDLAGLVKLVQPQEHAADSLVDVDLLSQVDPVVVQANEVDQAGVVQLKALVDQPAQGDKVAALPKQLDGVGGQKPADVAASLSAQRVLLFQPLQGHVQAGHAHSVHDGGIGGVQRQADLGLCGHDSSASTSARSSAARAADFSLLLRGVVWHVVGVHGGDLSPV

SEQ ID NO: 262 - DNA, 47 bp, Artificial Sequence; identical to SEQ ID NO: 167
  Synthetic

SEQ ID NO: 263 - DNA, 47 bp, Artificial Sequence; identical to SEQ ID NO: 167
  Synthetic

SEQ ID NO: 264 - DNA, 21 bp, Artificial Sequence
  Synthetic
cgcaaatgggcggtaggcgtg

SEQ ID NO: 265 - DNA, 49 bp, Artificial Sequence; identical to SEQ ID NO: 216
  Synthetic

SEQ ID NO: 266 - DNA, 45 bp, Artificial Sequence
  Synthetic
atctggcggaggtggaagcggaggaaccatctacagcgagcactg

SEQ ID NO: 267 - DNA, 20 bp, Artificial Sequence
  Synthetic
tcctccgcttccacctccgc

SEQ ID NO: 268 - DNA, 47 bp, Artificial Sequence; identical to SEQ ID NO: 167
  Synthetic

SEQ ID NO: 269 - DNA, 20 bp, Artificial Sequence
  Synthetic
ctagcataaccccttggggc

SEQ ID NO: 270 - DNA, 47 bp, Artificial Sequence; identical to SEQ ID NO: 167
  Synthetic

SEQ ID NO: 271 - DNA, 50 bp, Artificial Sequence
  Synthetic
agtgcgccccaaggggttatgctagtcacagtttcagttccagggagaag

SEQ ID NO: 272 - DNA, 20 bp, Artificial Sequence
  Synthetic
ttttggcagagggaaaaaga

SEQ ID NO: 273 - DNA, 1400 bp, Artificial Sequence
  Synthetic
ttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatcgccaccatgtacccctacgacgtgccagactacgcccctaagaagaagagaaaagtcggcggctctggcggcggaggatctggcggaggtggaagcggaggaaccatctacagcgagcactggtccctgaatcctctggaaatccctcagcggagccggctgtttagcctggaacctgtggctgtgggcacaccttatgccgagagcctgagcagctacctgcacagactggcccaggctcactgtctgacaagcgagaagctggtcatgggcgagatcgcccctctgatcctgaaggacgaggacaagagcgagctgctgagcaagaacctgagccatctgctgggcaacagcgacgccaagcctgccatcaatggcatgagagagatgaccgagaagctcgtgaccgtgctggaagaactgaccatgcggcaggacctgcggtttctgacactgctgtcttggaagggcatgatctacgacaagggcctgttccgcaactacagagcctggtgtccttgctgctgcgaggaatggatgcagaagaacaagacaatctacgagcccctgagctggtccttcaaggacgtggaattctgcctgatccacaagcagcggctgatcgaggaatgcagccattgtggcgctagactgcccgtgatggccagactttctcctgccggcttctgcagcagatgctatggctggctgggccaagagatcaagggcgaagaggaaatcgagaagtaccgcgtgaacatccagggcatctccgagctgattgccctgacacctcagctgggctacaagcccattcctatcgagctgacccggaaactgcagctgatcctgctggtgttcgagcaggccatcggcaaggatgtgaagctgctgggagatctcggcggcatcatggaaagcctgagaatcgccagcaccaccaaccagagccagccttaccacctggtcaagctgatcatccccgtgtgcgagaaggccaagatcagcgtgttccagctgttcggcagcgacttcaaagagctgggcaagatcctgtttggcaacttctccctggaactgaaactgtgactagcataaccccttggggcgcactcctcaggtgcaggctgcctatcagaaggtggtggctggtgtggccaatgccctggctcacaaataccactgagatctttttccctctgccaaaaattatggggacatcatgaagccccttgagcatctgactt

SEQ ID NO: 274 - DNA, 1400 bp, Artificial Sequence
  Synthetic
aagtcagatgctcaaggggcttcatgatgtccccataatttttggcagagggaaaaagatctcagtggtatttgtgagccagggcattggccacaccagccaccaccttctgataggcagcctgcacctgaggagtgcgccccaaggggttatgctagtcacagtttcagttccagggagaagttgccaaacaggatcttgcccagctctttgaagtcgctgccgaacagctggaacacgctgatcttggccttctcgcacacggggatgatcagcttgaccaggtggtaaggctggctctggttggtggtgctggcgattctcaggctttccatgatgccgccgagatctcccagcagcttcacatccttgccgatggcctgctcgaacaccagcaggatcagctgcagtttccgggtcagctcgataggaatgggcttgtagcccagctgaggtgtcagggcaatcagctcggagatgccctggatgttcacgcggtacttctcgatttcctcttcgcccttgatctcttggcccagccagccatagcatctgctgcagaagccggcaggagaaagtctggccatcacgggcagtctagcgccacaatggctgcattcctcgatcagccgctgcttgtggatcaggcagaattccacgtccttgaaggaccagctcaggggctcgtagattgtcttgttcttctgcatccattcctcgcagcagcaaggacaccaggctctgtagttgcggaacaggcccttgtcgtagatcatgcccttccaagacagcagtgtcagaaaccgcaggtcctgccgcatggtcagttcttccagcacggtcacgagcttctcggtcatctctctcatgccattgatggcaggcttggcgtcgctgttgcccagcagatggctcaggttcttgctcagcagctcgctcttgtcctcgtccttcaggatcagaggggcgatctcgcccatgaccagcttctcgcttgtcagacagtgagcctgggccagtctgtgcaggtagctgctcaggctctcggcataaggtgtgcccacagccacaggttccaggctaaacagccggctccgctgagggatttccagaggattcagggaccagtgctcgctgtagatggttcctccgcttccacctccgccagatcctccgccgccagagccgccgacttttctcttcttcttaggggcgtagtctggcacgtcgtaggggtacatggtggcgatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaa

SEQ ID NO: 275 - protein, 360 aa, Artificial Sequence; identical to SEQ ID NO: 91
  Synthetic

SEQ ID NO: 276 - protein, 226 aa, Artificial Sequence
  Synthetic
MAAFLDQPLLVDQAEFHVLEGPAQGLVDCLVLLHPFLAAARTPGSVVAEQALVVDHALPRQQCQKPQVLPHGQFFQHGHELLGHLSHAIDGRLGVAVAQQMAQVLAQQLALVLVLQDQRGDLAHDQLLACQTVSLGQSVQVAAQALGIRCAHSHRFQAKQPAPLRDFQRIQGPVLAVDGSSASTSARSSAARAADFSLLLRGVVWHVVGVHGGDLSPVALADLTVH

SEQ ID NO: 277 - protein, 176 aa, Artificial Sequence
  Synthetic
MLWLAGPRDQGRRGNREVPREHPGHLRADCPDTSAGLQAHSYRADPETAADPAGVRAGHRQGCEAAGRSRRHHGKPENRQHHQPEPALPPGQADHPRVREGQDQRVPAVRQRLQRAGQDPVWQLLPGTETVTSITPWGALLRCRLPIRRWWLVWPMPWLTNTTEIFFPLPKIMGTS

SEQ ID NO: 278 - DNA, 47 bp, Artificial Sequence; identical to SEQ ID NO: 167
  Synthetic

SEQ ID NO: 279 - DNA, 47 bp, Artificial Sequence; identical to SEQ ID NO: 167
  Synthetic

SEQ ID NO: 280 - DNA, 46 bp, Artificial Sequence
  Synthetic
ctagcgctaccggactcagatcgccaccatgtacccctacgacgtg

SEQ ID NO: 281 - DNA, 45 bp, Artificial Sequence
  Synthetic
atctggcggaggtggaagcggaggactggcctctgtgctggaaac

SEQ ID NO: 282 - DNA, 20 bp, Artificial Sequence
  Synthetic
tcctccgcttccacctccgc

SEQ ID NO: 283 - DNA, 47 bp, Artificial Sequence; identical to SEQ ID NO: 167
  Synthetic

SEQ ID NO: 284 - DNA, 20 bp, Artificial Sequence
  Synthetic
ctagcataaccccttggggc

SEQ ID NO: 285 - DNA, 47 bp, Artificial Sequence; identical to SEQ ID NO: 167
  Synthetic

SEQ ID NO: 286 - DNA, 48 bp, Artificial Sequence
  Synthetic
agtgcgccccaaggggttatgctagtcacaggccaaattctctccgag

SEQ ID NO: 287 - DNA, 1536 bp, Artificial Sequence
  Synthetic
ctaccggactcagatcgccaccatgtacccctacgacgtgccagactacgcccctaagaagaagagaaaagtcggcggctctggcggcggaggatctggcggaggtggaagcggaggactggcctctgtgctggaaacctacgagagctggaacctgaagaagcctctgatccctcagcggagcagactgtaccagcctcagcctgttggcatcggcaccccttacatcgagagcctgaccggctacatcaccagaatcgccgaactgcatggcgtgctgcctggggtgctgatgaccagagaaatcgcccctctggtcaacaagatctacttccagaacggcgccaaccggggcttcagagagatcttcaacagatctcaggccctgaacggcatgggagagatggccgctgatctggtgcaggtcctgcagaagctgaccctgagggacgacctgagattcctgaccatgctgttctggtccaacatcctgacacctcggaacctgttccggaccagaaaggcctggtgtcccatctgctaccaagagcggcaccagaacggactggtggtgtacgaacagctgctgtggaccatcaacctgatcaccatctgtccccagcaccagaaacctctggtggaactgtgccctcactgcaaccacgagagccctctgctgaattggagaagcagacccggctactgcagcaagtgtggcgaatggctgggcgccaatcagtgcctgaaaaccttcacagatggcgagggcagcatcaaactgcagctggaatggcagtactggaccgccaatgttgtgggcgagctgatcctggccagccagtgttttgagagcgcccctagcaaagagaacatcaccaagagcctgaacatcgtgatcgacaaggtggccgagaacaacgccgctgccttctctagactgatcggcgtgcccaagaacagcctgtggatgtggcagagcaccaagacactgcccgagctgaacaccctgctgaagatctgctacgagctggaaatcagcctggtcgagtttctgacccctaagaacctgattaccaagtccttcaccaagatcagccagaagcacctccagctgagcagaacccctagagtgtcccctaagagcttcgaccagtaccaagtgaaggacgccctgctggccatcctggccggaaatgaagaacctcctccaaccatggaagaagtgggcaagagactgggccaccacaaccggacaatcagcagacacttccccgatctgtgcagcgccatcagcgccaagtgccggaattacaacaaggcctgccggctgaagtccatcgagaagctgtgtagcgaagtgcgcgagatcgtgctgagcctgaatgctcagggcgtgtaccctacagagggcagagtgtgcgaactgatgcccaatcctggctgcttccggtacaagcaagtgcgggctgccttcaacgacgctcggagagaatttggcctgtgactagcataaccccttggggcgcactcctcagg

SEQ ID NO: 288 - DNA, 1536 bp, Artificial Sequence
  Synthetic
cctgaggagtgcgccccaaggggttatgctagtcacaggccaaattctctccgagcgtcgttgaaggcagcccgcacttgcttgtaccggaagcagccaggattgggcatcagttcgcacactctgccctctgtagggtacacgccctgagcattcaggctcagcacgatctcgcgcacttcgctacacagcttctcgatggacttcagccggcaggccttgttgtaattccggcacttggcgctgatggcgctgcacagatcggggaagtgtctgctgattgtccggttgtggtggcccagtctcttgcccacttcttccatggttggaggaggttcttcatttccggccaggatggccagcagggcgtccttcacttggtactggtcgaagctcttaggggacactctaggggttctgctcagctggaggtgcttctggctgatcttggtgaaggacttggtaatcaggttcttaggggtcagaaactcgaccaggctgatttccagctcgtagcagatcttcagcagggtgttcagctcgggcagtgtcttggtgctctgccacatccacaggctgttcttgggcacgccgatcagtctagagaaggcagcggcgttgttctcggccaccttgtcgatcacgatgttcaggctcttggtgatgttctctttgctaggggcgctctcaaaacactggctggccaggatcagctcgcccacaacattggcggtccagtactgccattccagctgcagtttgatgctgccctcgccatctgtgaaggttttcaggcactgattggcgcccagccattcgccacacttgctgcagtagccgggtctgcttctccaattcagcagagggctctcgtggttgcagtgagggcacagttccaccagaggtttctggtgctggggacagatggtgatcaggttgatggtccacagcagctgttcgtacaccaccagtccgttctggtgccgctcttggtagcagatgggacaccaggcctttctggtccggaacaggttccgaggtgtcaggatgttggaccagaacagcatggtcaggaatctcaggtcgtccctcagggtcagcttctgcaggacctgcaccagatcagcggccatctctcccatgccgttcagggcctgagatctgttgaagatctctctgaagccccggttggcgccgttctggaagtagatcttgttgaccagaggggcgatttctctggtcatcagcaccccaggcagcacgccatgcagttcggcgattctggtgatgtagccggtcaggctctcgatgtaaggggtgccgatgccaacaggctgaggctggtacagtctgctccgctgagggatcagaggcttcttcaggttccagctctcgtaggtttccagcacagaggccagtcctccgcttccacctccgccagatcctccgccgccagagccgccgacttttctcttcttcttaggggcgtagtctggcacgtcgtaggggtacatggtggcgatctgagtccggtag

SEQ ID NO: 289 - protein, 493 aa, Artificial Sequence; identical to SEQ ID NO: 92
  Synthetic

SEQ ID NO: 290 - DNA, 47 bp, Artificial Sequence; identical to SEQ ID NO: 167
  Synthetic

SEQ ID NO: 291 - DNA, 47 bp, Artificial Sequence
  Synthetic
gctagcgctaccggactcagatcgccaccatgtacccctacgacgtg

SEQ ID NO: 292 - DNA, 45 bp, Artificial Sequence
  Synthetic
atctggcggaggtggaagcggaggaaaggaacagttcgagcagtg

SEQ ID NO: 293 - DNA, 20 bp, Artificial Sequence
  Synthetic
tcctccgcttccacctccgc

SEQ ID NO: 294 - DNA, 20 bp, Artificial Sequence
  Synthetic
ctagcataaccccttggggc

SEQ ID NO: 295 - DNA, 47 bp, Artificial Sequence; identical to SEQ ID NO: 167
  Synthetic

SEQ ID NO: 296 - DNA, 46 bp, Artificial Sequence
  Synthetic
agtgcgccccaaggggttatgctagtcagaattcctcgtacaccac

SEQ ID NO: 297 - DNA, 2855 bp, Artificial Sequence
  Synthetic
gctagcgctaccggactcagatcgccaccatgtacccctacgacgtgccagactacgcccctaagaagaagagaaaagtcggcggctctggcggcggaggatctggcggaggtggaagcggaggaaaggaacagttcgagcagtgggtcgcccggctgcagatttctgaagccggccagaaaatcatcgagaaagtgcggagcagcgagcccagcagaagagttggcggaggcagcaagaatgtgtccggcagatacccctccagaaagatgggcgtgaccatccagttcgagagccacagagtggaactgcccatcatctaccagctggaacacgacaaggacgtgctggaattctacgaccagcctcctcagatcaagctggactaccaggccagcaatggcagacgactgggagtgctgcacacccctgatctgttcgtgatcaggaccaatagcgccggctgggaagagtgcaagaccgagcaggatctgaagaagctggccgagaagaaccccaaccggtacttctacagcgaagagaacaaccagtggcacagcctgcctggcgaggtgtacgccaatcagtttggcctgtaccaccggatccacagcgaccgcgaaatcaactgggtgctgcagcggaacctgcagttcctggaagattactaccgcagcgagagcctggtggtggaagaggccattgctcagtctctgctggccatcgtgtctagccagcctggaattacactggccgagctgctgaacagaagcaccggcgccaagagcgacgacatctacaccctgatcatccaagagcagatctacatcgacctgaacgccacacctctggctgagcctgagagatgcctgatcttcagggatgagcagaccgccagcgcctacagactgatggttgagcagcctagcgtgtccctgcctgccatctctcccgtggtcaatattgtgacaggcgccaccgtgtcctgggatggcaagggcctgaatatcatccacgtgggcgagacagaagtgatcctgggcgccgagaacaatcagctcatcgaactgaagaaggccatcttcgagaacctcgtgcggcagggcaagatcacctctctgcagaccccagagaaaaccgccatcagcacagagagctggcagcggtttcatcaggcctctccagaggatcaggccgaggctctggaacggtacaagaccatcgagccctacctgaacggacaccctcctgagaacgagacaatccccgccagaaccatcagacactggaaggccaagtacctgatcgccatccagaagtacggctgcggctatatcggcctgctgtcccacagaagcgtgaagggcaacagacagagaaagctgcccgaggacaccctggccatcatggaaaagttcattctcgaggactacgagacactgaagcagaaacggatgtgggaagtgcacgccgctctcgtgcgagcttgtgaagaagcaggcgttatcgcccctagctacaaggccttcaccaaagaggtgcagcggagaaccggctacgagcagaccaagaagagacaaggcagaagggccgcctaccagcacgagtccttttactgggaactcgccatcaccacacctcggcacggcgatagacctttcgagatcggccacatcgaccacaccgagctggatgtggaactcgtgtgtagcgacaccggcagaaacctgggtagaccttgggccacctttctggtggacgcctactctagaaggctgctggccgtgtacctgaccttcgacagcccaagctaccggtcctgtctcatggtgctgcggatctgcgtgaagaggcatggcagactgcctcagatcgtggtggtcgataatggcgccgagtttcacagcgtgtacttcgaaaccctgctggctaccttcgagtgcaccaagaagcagcggcctccagccaaggccagatttggcagcgtgtgcgagagactgttcggcaccagcaacacccagttcgtgcacaacctgctgggcaatacccagatcacccggaacgtgcggcaagtgaccaagagcgtgaaccccaagaacctggctgtgtggacactgggactgctgtacgagtacctgtgtgcctgggcctacgaagtgtacgataccgacgaacaccccgctctgtttcagagccctagggatgcctttgccgccggaatggctatcggcggatctcgggtgcacagaatgatcccttacgacgagaacttccagatcctgacactgcccaccacctccgagggcaaagccaaggtgcaagtcggcagaggcatcaagatcaactacatctactactggtccaacagcttcagggacccacagatcgagaacaccagcgtgcagatcagatacgaccccttcaacatcggaatcgcctacgccttcgtcagaggccagtgggtgcagtgcatcagccagtactacgccgaactccagggcagaagcgagaaagagctgaaactggccagcatcgagctgcggaagcactacagcaatcacacccagcagagcaaggtgtccgccaaaaacctggccgagttcctggcaagcgtggaagctcaagaagccctgctggaacagagaagctacgacgccgaagtgaaagaggtgttccgggtcatcgaaggcggcaaggccacaaccagcagaaatgaggaacccaagctgatccaagtgaccttcgccaacaccgacttccaggccgatgaggacgaagccatcgtgcctgaaacactggtggtgtacgaggaattctgactagcataaccccttggggcgcactcctcaggtgca

SEQ ID NO: 298 - DNA, 2855 bp, Artificial Sequence
  Synthetic
tgcacctgaggagtgcgccccaaggggttatgctagtcagaattcctcgtacaccaccagtgtttcaggcacgatggcttcgtcctcatcggcctggaagtcggtgttggcgaaggtcacttggatcagcttgggttcctcatttctgctggttgtggccttgccgccttcgatgacccggaacacctctttcacttcggcgtcgtagcttctctgttccagcagggcttcttgagcttccacgcttgccaggaactcggccaggtttttggcggacaccttgctctgctgggtgtgattgctgtagtgcttccgcagctcgatgctggccagtttcagctctttctcgcttctgccctggagttcggcgtagtactggctgatgcactgcacccactggcctctgacgaaggcgtaggcgattccgatgttgaaggggtcgtatctgatctgcacgctggtgttctcgatctgtgggtccctgaagctgttggaccagtagtagatgtagttgatcttgatgcctctgccgacttgcaccttggctttgccctcggaggtggtgggcagtgtcaggatctggaagttctcgtcgtaagggatcattctgtgcacccgagatccgccgatagccattccggcggcaaaggcatccctagggctctgaaacagagcggggtgttcgtcggtatcgtacacttcgtaggcccaggcacacaggtactcgtacagcagtcccagtgtccacacagccaggttcttggggttcacgctcttggtcacttgccgcacgttccgggtgatctgggtattgcccagcaggttgtgcacgaactgggtgttgctggtgccgaacagtctctcgcacacgctgccaaatctggccttggctggaggccgctgcttcttggtgcactcgaaggtagccagcagggtttcgaagtacacgctgtgaaactcggcgccattatcgaccaccacgatctgaggcagtctgccatgcctcttcacgcagatccgcagcaccatgagacaggaccggtagcttgggctgtcgaaggtcaggtacacggccagcagccttctagagtaggcgtccaccagaaaggtggcccaaggtctacccaggtttctgccggtgtcgctacacacgagttccacatccagctcggtgtggtcgatgtggccgatctcgaaaggtctatcgccgtgccgaggtgtggtgatggcgagttcccagtaaaaggactcgtgctggtaggcggcccttctgccttgtctcttcttggtctgctcgtagccggttctccgctgcacctctttggtgaaggccttgtagctaggggcgataacgcctgcttcttcacaagctcgcacgagagcggcgtgcacttcccacatccgtttctgcttcagtgtctcgtagtcctcgagaatgaacttttccatgatggccagggtgtcctcgggcagctttctctgtctgttgcccttcacgcttctgtgggacagcaggccgatatagccgcagccgtacttctggatggcgatcaggtacttggccttccagtgtctgatggttctggcggggattgtctcgttctcaggagggtgtccgttcaggtagggctcgatggtcttgtaccgttccagagcctcggcctgatcctctggagaggcctgatgaaaccgctgccagctctctgtgctgatggcggttttctctggggtctgcagagaggtgatcttgccctgccgcacgaggttctcgaagatggccttcttcagttcgatgagctgattgttctcggcgcccaggatcacttctgtctcgcccacgtggatgatattcaggcccttgccatcccaggacacggtggcgcctgtcacaatattgaccacgggagagatggcaggcagggacacgctaggctgctcaaccatcagtctgtaggcgctggcggtctgctcatccctgaagatcaggcatctctcaggctcagccagaggtgtggcgttcaggtcgatgtagatctgctcttggatgatcagggtgtagatgtcgtcgctcttggcgccggtgcttctgttcagcagctcggccagtgtaattccaggctggctagacacgatggccagcagagactgagcaatggcctcttccaccaccaggctctcgctgcggtagtaatcttccaggaactgcaggttccgctgcagcacccagttgatttcgcggtcgctgtggatccggtggtacaggccaaactgattggcgtacacctcgccaggcaggctgtgccactggttgttctcttcgctgtagaagtaccggttggggttcttctcggccagcttcttcagatcctgctcggtcttgcactcttcccagccggcgctattggtcctgatcacgaacagatcaggggtgtgcagcactcccagtcgtctgccattgctggcctggtagtccagcttgatctgaggaggctggtcgtagaattccagcacgtccttgtcgtgttccagctggtagatgatgggcagttccactctgtggctctcgaactggatggtcacgcccatctttctggaggggtatctgccggacacattcttgctgcctccgccaactcttctgctgggctcgctgctccgcactttctcgatgattttctggccggcttcagaaatctgcagccgggcgacccactgctcgaactgttcctttcctccgcttccacctccgccagatcctccgccgccagagccgccgacttttctcttcttcttaggggcgtagtctggcacgtcgtaggggtacatggtggcgatctgagtccggtagcgctagc

SEQ ID NO: 299 - DNA, 47 bp, Artificial Sequence; identical to SEQ ID NO: 167
  Synthetic

SEQ ID NO: 300 - protein, 929 aa, Artificial Sequence; identical to SEQ ID NO: 93
  Synthetic

SEQ ID NO: 301 - protein, 619 aa, Artificial Sequence
  Synthetic
MPLHADPQHHETGPVAWAVEGQVHGQQPSRVGVHQKGGPRSTQVSAGVATHEFHIQLGVVDVADLERSIAVPRCGDGEFPVKGLVLVGGPSALSLLGLLVAGSPLHLFGEGLVARGDNACFFTSSHESGVHFPHPFLLQCLVVLENELFHDGQGVLGQLSLSVALHASVGQQADIAAAVLLDGDQVLGLPVSDGSGGDCLVLRRVSVQVGLDGLVPFQSLGLILWRGLMKPLPALCADGGFLWGLQRGDLALPHEVLEDGLLQFDELIVLGAQDHFCLAHVDDIQALAIPGHGGACHNIDHGRDGRQGHARLLNHQSVGAGGLLIPEDQASLRLSQRCGVQVDVDLLLDDQGVDVVALGAGASVQQLGQCNSRLARHDGQQRLSNGLFHHQALAAVVIFQELQVPLQHPVDFAVAVDPVVQAKLIGVHLARQAVPLVVLFAVEVPVGVLLGQLLQILLGLALFPAGAIGPDHEQIRGVQHSQSSAIAGLVVQLDLRRLVVEFQHVLVVFQLVDDGQFHSVALELDGHAHLSGGVSAGHILAASANSSAGLAAPHFLDDFLAGFRNLQPGDPLLELFLSSASTSARSSAARAADFSLLLRGVVWHVVGVHGGDLSPVALA

SEQ ID NO: 302 - DNA, 47 bp, Artificial Sequence; identical to SEQ ID NO: 167
  Synthetic

SEQ ID NO: 303 - DNA, 47 bp, Artificial Sequence; identical to SEQ ID NO: 167
  Synthetic

SEQ ID NO: 304 - DNA, 36 bp, Artificial Sequence
  Synthetic
cggactcagatcgccaccatgtacccctacgacgtg

SEQ ID NO: 305 - DNA, 47 bp, Artificial Sequence; identical to SEQ ID NO: 167
  Synthetic

SEQ ID NO: 306 - DNA, 44 bp, Artificial Sequence
  Synthetic
atctggcggaggtggaagcggaggaaccaagagcaccggctttc

SEQ ID NO: 307 - DNA, 20 bp, Artificial Sequence
  Synthetic
tcctccgcttccacctccgc

SEQ ID NO: 308 - DNA, 41 bp, Artificial Sequence
  Synthetic
cccaaggggttatgctagtcaagacacatcctgctgcactc

SEQ ID NO: 309 - DNA, 19 bp, Artificial Sequence
  Synthetic
ctagcataaccccttgggg

SEQ ID NO: 310 - DNA, 12 bp, Artificial Sequence
  Synthetic
atgagccagatc

SEQ ID NO: 311 - DNA, 1281 bp, Artificial Sequence
  Synthetic
cggactcagatcgccaccatgtacccctacgacgtgccagactacgcccctaagaagaagagaaaagtcggcggctctggcggcggaggatctggcggaggtggaagcggaggaaccaagagcaccggctttccactggaactgctgaccagacctgccaccgagagactggcctacttcgagaactacaccgtggctcaccccagactgaaagaggtgtacgagatcctgatgcggacaatcgccgaacctgccggcgctagcttcatctttgtgtatggcgctagcggcgtgggcaagaccacactgagactgagagtggaacagaagctgaccgagctggccctgcctaagctggaaagcgatagagccagagtgcccgtcgtgggcattgaggctattgcccctgagagccggtacttcaactggaaagagtactacacacgggccctgatcaccctggaagaacccctgatcgaccacaagttcgactatggcgtgcggggcatcagcagagacaacttcggcaagatcaacgtggaaagcaaggtggtggcccctgctctgagaagggccctcgaaaatgccctgatccacagacaccccgacgtgttctttgtggacgaggcccagcactttggcaaggtggcctctggctacaagctgcaggatcagctggactgcctgaagtccctggccaacatgacaggcatcctgcactgtctgctgggcacctacgagctgctgaccttcagaaatctgagcggccagctgtctcggcggagcgtggacatccacttcagaagatactgcgccgactctcccgaggacgtgcaggcctttaaaagcgtgctgctgacattccagcagcatctgcctctggccgagacacccaatctggtggatcactgggagtacttctacgagagaaccctgggctgcatcggcaccctgaaggactggctgaagagagtgctgagcgacgccctggatagagaggccacaaccatcacactgaaggacctgcagaaaagggccctgagcgtggcccagtgccagaagatgttcaaagagatccaagagggcgaacgccagctgagcgaaacagaggccgatgtgcagaacctgagatctgcccttggcctgggagccaagcctatcgttctgcctgaggaaacccctaagaccaccagacctccaggcaaagtgggcaaaagaaagcccaagagggaccccatcggagtgcagcaggatgtgtcttgactagcataaccccttggg

SEQ ID NO: 312 - DNA, 1281 bp, Artificial Sequence
  Synthetic
cccaaggggttatgctagtcaagacacatcctgctgcactccgatggggtccctcttgggctttcttttgcccactttgcctggaggtctggtggtcttaggggtttcctcaggcagaacgataggcttggctcccaggccaagggcagatctcaggttctgcacatcggcctctgtttcgctcagctggcgttcgccctcttggatctctttgaacatcttctggcactgggccacgctcagggcccttttctgcaggtccttcagtgtgatggttgtggcctctctatccagggcgtcgctcagcactctcttcagccagtccttcagggtgccgatgcagcccagggttctctcgtagaagtactcccagtgatccaccagattgggtgtctcggccagaggcagatgctgctggaatgtcagcagcacgcttttaaaggcctgcacgtcctcgggagagtcggcgcagtatcttctgaagtggatgtccacgctccgccgagacagctggccgctcagatttctgaaggtcagcagctcgtaggtgcccagcagacagtgcaggatgcctgtcatgttggccagggacttcaggcagtccagctgatcctgcagcttgtagccagaggccaccttgccaaagtgctgggcctcgtccacaaagaacacgtcggggtgtctgtggatcagggcattttcgagggcccttctcagagcaggggccaccaccttgctttccacgttgatcttgccgaagttgtctctgctgatgccccgcacgccatagtcgaacttgtggtcgatcaggggttcttccagggtgatcagggcccgtgtgtagtactctttccagttgaagtaccggctctcaggggcaatagcctcaatgcccacgacgggcactctggctctatcgctttccagcttaggcagggccagctcggtcagcttctgttccactctcagtctcagtgtggtcttgcccacgccgctagcgccatacacaaagatgaagctagcgccggcaggttcggcgattgtccgcatcaggatctcgtacacctctttcagtctggggtgagccacggtgtagttctcgaagtaggccagtctctcggtggcaggtctggtcagcagttccagtggaaagccggtgctcttggttcctccgcttccacctccgccagatcctccgccgccagagccgccgacttttctcttcttcttaggggcgtagtctggcacgtcgtaggggtacatggtggcgatctgagtccg

SEQ ID NO: 313 - protein, 414 aa, Artificial Sequence; identical to SEQ ID NO: 94
  Synthetic

SEQ ID NO: 314 - protein, 291 aa, Artificial Sequence
  Synthetic
MLLECQQHAFKGLHVLGRVGAVSSEVDVHAPPRQLAAQISEGQQLVGAQQTVQDACHVGQGLQAVQLILQLVARGHLAKVLGLVHKEHVGVSVDQGIFEGPSQSRGHHLAFHVDLAEVVSADAPHAIVELVVDQGFFQGDQGPCVVLFPVEVPALRGNSLNAHDGHSGSIAFQLRQGQLGQLLFHSQSQCGLAHAASAIHKDEASAGRFGDCPHQDLVHLFQSGVSHGVVLEVGQSLGGRSGQQFQWKAGALGSSASTSARSSAARAADFSLLLRGVVWHVVGVHGGDLSP

SEQ ID NO: 315 - DNA, 45 bp, Artificial Sequence
  Synthetic
atgagccagatcaccatccaggccagactgatcagcttcgagcaa

SEQ ID NO: 316 - DNA, 47 bp, Artificial Sequence; identical to SEQ ID NO: 167
  Synthetic

SEQ ID NO: 317 - DNA, 45 bp, Artificial Sequence
  Synthetic
ctaccggactcagatcgccaccatgaccatctacagcgagcactg

SEQ ID NO: 318 - DNA, 47 bp, Artificial Sequence; identical to SEQ ID NO: 167
  Synthetic

SEQ ID NO: 319 - DNA, 21 bp, Artificial Sequence
  Synthetic
catggtggcgatctgagtccg

SEQ ID NO: 320 - DNA, 19 bp, Artificial Sequence
  Synthetic
ggaggatctggcggaggcg

SEQ ID NO: 321 - DNA, 50 bp, Artificial Sequence
  Synthetic
cagagccgcctccgccagatcctcccagtttcagttccagggagaagttg

SEQ ID NO: 322 - DNA, 21 bp, Artificial Sequence
  Synthetic
ccccaaggggttatgctagtc

SEQ ID NO: 323 - DNA, 47 bp, Artificial Sequence; identical to SEQ ID NO: 167
  Synthetic

SEQ ID NO: 324 - DNA, 1141 bp, Artificial Sequence
  Synthetic
cgtcagatccgctagcgctaccggactcagatcgccaccatgaccatctacagcgagcactggtccctgaatcctctggaaatccctcagcggagccggctgtttagcctggaacctgtggctgtgggcacaccttatgccgagagcctgagcagctacctgcacagactggcccaggctcactgtctgacaagcgagaagctggtcatgggcgagatcgcccctctgatcctgaaggacgaggacaagagcgagctgctgagcaagaacctgagccatctgctgggcaacagcgacgccaagcctgccatcaatggcatgagagagatgaccgagaagctcgtgaccgtgctggaagaactgaccatgcggcaggacctgcggtttctgacactgctgtcttggaagggcatgatctacgacaagggcctgttccgcaactacagagcctggtgtccttgctgctgcgaggaatggatgcagaagaacaagacaatctacgagcccctgagctggtccttcaaggacgtggaattctgcctgatccacaagcagcggctgatcgaggaatgcagccattgtggcgctagactgcccgtgatggccagactttctcctgccggcttctgcagcagatgctatggctggctgggccaagagatcaagggcgaagaggaaatcgagaagtaccgcgtgaacatccagggcatctccgagctgattgccctgacacctcagctgggctacaagcccattcctatcgagctgacccggaaactgcagctgatcctgctggtgttcgagcaggccatcggcaaggatgtgaagctgctgggagatctcggcggcatcatggaaagcctgagaatcgccagcaccaccaaccagagccagccttaccacctggtcaagctgatcatccccgtgtgcgagaaggccaagatcagcgtgttccagctgttcggcagcgacttcaaagagctgggcaagatcctgtttggcaacttctccctggaactgaaactgggaggatctggcggaggcggctctggcggcggtggaagcggaggacctaagaagaagagaaaagtctacccctacgacgtgccagactacgcctgactagcataaccccttgggg

SEQ ID NO: 325 - DNA, 1141 bp, Artificial Sequence
  Synthetic
ccccaaggggttatgctagtcaggcgtagtctggcacgtcgtaggggtagacttttctcttcttcttaggtcctccgcttccaccgccgccagagccgcctccgccagatcctcccagtttcagttccagggagaagttgccaaacaggatcttgcccagctctttgaagtcgctgccgaacagctggaacacgctgatcttggccttctcgcacacggggatgatcagcttgaccaggtggtaaggctggctctggttggtggtgctggcgattctcaggctttccatgatgccgccgagatctcccagcagcttcacatccttgccgatggcctgctcgaacaccagcaggatcagctgcagtttccgggtcagctcgataggaatgggcttgtagcccagctgaggtgtcagggcaatcagctcggagatgccctggatgttcacgcggtacttctcgatttcctcttcgcccttgatctcttggcccagccagccatagcatctgctgcagaagccggcaggagaaagtctggccatcacgggcagtctagcgccacaatggctgcattcctcgatcagccgctgcttgtggatcaggcagaattccacgtccttgaaggaccagctcaggggctcgtagattgtcttgttcttctgcatccattcctcgcagcagcaaggacaccaggctctgtagttgcggaacaggcccttgtcgtagatcatgcccttccaagacagcagtgtcagaaaccgcaggtcctgccgcatggtcagttcttccagcacggtcacgagcttctcggtcatctctctcatgccattgatggcaggcttggcgtcgctgttgcccagcagatggctcaggttcttgctcagcagctcgctcttgtcctcgtccttcaggatcagaggggcgatctcgcccatgaccagcttctcgcttgtcagacagtgagcctgggccagtctgtgcaggtagctgctcaggctctcggcataaggtgtgcccacagccacaggttccaggctaaacagccggctccgctgagggatttccagaggattcagggaccagtgctcgctgtagatggtcatggtggcgatctgagtccggtagcgctagcggatctgacg

SEQ ID NO: 326 - protein, 360 aa, Artificial Sequence; identical to SEQ ID NO: 95
  Synthetic

SEQ ID NO: 327 - protein, 190 aa, Artificial Sequence
  Synthetic
MAAFLDQPLLVDQAEFHVLEGPAQGLVDCLVLLHPFLAAARTPGSVVAEQALVVDHALPRQQCQKPQVLPHGQFFQHGHELLGHLSHAIDGRLGVAVAQQMAQVLAQQLALVLVLQDQRGDLAHDQLLACQTVSLGQSVQVAAQALGIRCAHSHRFQAKQPAPLRDFQRIQGPVLAVDGHGGDLSPVALA

SEQ ID NO: 328 - DNA, 47 bp, Artificial Sequence; identical to SEQ ID NO: 167
  Synthetic

SEQ ID NO: 329 - DNA, 47 bp, Artificial Sequence; identical to SEQ ID NO: 167
  Synthetic

SEQ ID NO: 330 - DNA, 42 bp, Artificial Sequence
  Synthetic
ccggactcagatcgccaccatgctggcctctgtgctggaaac

SEQ ID NO: 331 - DNA, 21 bp, Artificial Sequence
  Synthetic
catggtggcgatctgagtccg

SEQ ID NO: 332 - DNA, 19 bp, Artificial Sequence
  Synthetic
ggaggatctggcggaggcg

SEQ ID NO: 333 - DNA, 46 bp, Artificial Sequence
  Synthetic
cagagccgcctccgccagatcctcccaggccaaattctctccgagc

SEQ ID NO: 334 - DNA, 23 bp, Artificial Sequence
  Synthetic
cgccccaaggggttatgctagtc

SEQ ID NO: 335 - DNA, 47 bp, Artificial Sequence; identical to SEQ ID NO: 167
  Synthetic

SEQ ID NO: 336 - DNA, 1522 bp, Artificial Sequence
  Synthetic
ccggactcagatcgccaccatgctggcctctgtgctggaaacctacgagagctggaacctgaagaagcctctgatccctcagcggagcagactgtaccagcctcagcctgttggcatcggcaccccttacatcgagagcctgaccggctacatcaccagaatcgccgaactgcatggcgtgctgcctggggtgctgatgaccagagaaatcgcccctctggtcaacaagatctacttccagaacggcgccaaccggggcttcagagagatcttcaacagatctcaggccctgaacggcatgggagagatggccgctgatctggtgcaggtcctgcagaagctgaccctgagggacgacctgagattcctgaccatgctgttctggtccaacatcctgacacctcggaacctgttccggaccagaaaggcctggtgtcccatctgctaccaagagcggcaccagaacggactggtggtgtacgaacagctgctgtggaccatcaacctgatcaccatctgtccccagcaccagaaacctctggtggaactgtgccctcactgcaaccacgagagccctctgctgaattggagaagcagacccggctactgcagcaagtgtggcgaatggctgggcgccaatcagtgcctgaaaaccttcacagatggcgagggcagcatcaaactgcagctggaatggcagtactggaccgccaatgttgtgggcgagctgatcctggccagccagtgttttgagagcgcccctagcaaagagaacatcaccaagagcctgaacatcgtgatcgacaaggtggccgagaacaacgccgctgccttctctagactgatcggcgtgcccaagaacagcctgtggatgtggcagagcaccaagacactgcccgagctgaacaccctgctgaagatctgctacgagctggaaatcagcctggtcgagtttctgacccctaagaacctgattaccaagtccttcaccaagatcagccagaagcacctccagctgagcagaacccctagagtgtcccctaagagcttcgaccagtaccaagtgaaggacgccctgctggccatcctggccggaaatgaagaacctcctccaaccatggaagaagtgggcaagagactgggccaccacaaccggacaatcagcagacacttccccgatctgtgcagcgccatcagcgccaagtgccggaattacaacaaggcctgccggctgaagtccatcgagaagctgtgtagcgaagtgcgcgagatcgtgctgagcctgaatgctcagggcgtgtaccctacagagggcagagtgtgcgaactgatgcccaatcctggctgcttccggtacaagcaagtgcgggctgccttcaacgacgctcggagagaatttggcctgggaggatctggcggaggcggctctggcggcggtggaagcggaggacctaagaagaagagaaaagtctacccctacgacgtgccagactacgcctgactagcataaccccttggggcg

SEQ ID NO: 337 - DNA, 1522 bp, Artificial Sequence
  Synthetic
cgccccaaggggttatgctagtcaggcgtagtctggcacgtcgtaggggtagacttttctcttcttcttaggtcctccgcttccaccgccgccagagccgcctccgccagatcctcccaggccaaattctctccgagcgtcgttgaaggcagcccgcacttgcttgtaccggaagcagccaggattgggcatcagttcgcacactctgccctctgtagggtacacgccctgagcattcaggctcagcacgatctcgcgcacttcgctacacagcttctcgatggacttcagccggcaggccttgttgtaattccggcacttggcgctgatggcgctgcacagatcggggaagtgtctgctgattgtccggttgtggtggcccagtctcttgcccacttcttccatggttggaggaggttcttcatttccggccaggatggccagcagggcgtccttcacttggtactggtcgaagctcttaggggacactctaggggttctgctcagctggaggtgcttctggctgatcttggtgaaggacttggtaatcaggttcttaggggtcagaaactcgaccaggctgatttccagctcgtagcagatcttcagcagggtgttcagctcgggcagtgtcttggtgctctgccacatccacaggctgttcttgggcacgccgatcagtctagagaaggcagcggcgttgttctcggccaccttgtcgatcacgatgttcaggctcttggtgatgttctctttgctaggggcgctctcaaaacactggctggccaggatcagctcgcccacaacattggcggtccagtactgccattccagctgcagtttgatgctgccctcgccatctgtgaaggttttcaggcactgattggcgcccagccattcgccacacttgctgcagtagccgggtctgcttctccaattcagcagagggctctcgtggttgcagtgagggcacagttccaccagaggtttctggtgctggggacagatggtgatcaggttgatggtccacagcagctgttcgtacaccaccagtccgttctggtgccgctcttggtagcagatgggacaccaggcctttctggtccggaacaggttccgaggtgtcaggatgttggaccagaacagcatggtcaggaatctcaggtcgtccctcagggtcagcttctgcaggacctgcaccagatcagcggccatctctcccatgccgttcagggcctgagatctgttgaagatctctctgaagccccggttggcgccgttctggaagtagatcttgttgaccagaggggcgatttctctggtcatcagcaccccaggcagcacgccatgcagttcggcgattctggtgatgtagccggtcaggctctcgatgtaaggggtgccgatgccaacaggctgaggctggtacagtctgctccgctgagggatcagaggcttcttcaggttccagctctcgtaggtttccagcacagaggccagcatggtggcgatctgagtccgg

SEQ ID NO: 338 - protein, 493 aa, Artificial Sequence; identical to SEQ ID NO: 96
  Synthetic

SEQ ID NO: 339 - DNA, 47 bp, Artificial Sequence; identical to SEQ ID NO: 167
  Synthetic

SEQ ID NO: 340 - DNA, 47 bp, Artificial Sequence; identical to SEQ ID NO: 167
  Synthetic

SEQ ID NO: 341 - DNA, 45 bp, Artificial Sequence
  Synthetic
ctaccggactcagatcgccaccatgaaggaacagttcgagcagtg

SEQ ID NO: 342 - DNA, 21 bp, Artificial Sequence
  Synthetic
catggtggcgatctgagtccg

SEQ ID NO: 343 - DNA, 19 bp, Artificial Sequence
  Synthetic
ggaggatctggcggaggcg

SEQ ID NO: 344 - DNA, 44 bp, Artificial Sequence
  Synthetic
cagagccgcctccgccagatcctccgaattcctcgtacaccacc

SEQ ID NO: 345 - DNA, 24 bp, Artificial Sequence
  Synthetic
gcgccccaaggggttatgctagtc

SEQ ID NO: 346 - DNA, 2856 bp, Artificial Sequence
  Synthetic
tgaaccgtcagatccgctagcgctaccggactcagatcgccaccatgaaggaacagttcgagcagtgggtcgcccggctgcagatttctgaagccggccagaaaatcatcgagaaagtgcggagcagcgagcccagcagaagagttggcggaggcagcaagaatgtgtccggcagatacccctccagaaagatgggcgtgaccatccagttcgagagccacagagtggaactgcccatcatctaccagctggaacacgacaaggacgtgctggaattctacgaccagcctcctcagatcaagctggactaccaggccagcaatggcagacgactgggagtgctgcacacccctgatctgttcgtgatcaggaccaatagcgccggctgggaagagtgcaagaccgagcaggatctgaagaagctggccgagaagaaccccaaccggtacttctacagcgaagagaacaaccagtggcacagcctgcctggcgaggtgtacgccaatcagtttggcctgtaccaccggatccacagcgaccgcgaaatcaactgggtgctgcagcggaacctgcagttcctggaagattactaccgcagcgagagcctggtggtggaagaggccattgctcagtctctgctggccatcgtgtctagccagcctggaattacactggccgagctgctgaacagaagcaccggcgccaagagcgacgacatctacaccctgatcatccaagagcagatctacatcgacctgaacgccacacctctggctgagcctgagagatgcctgatcttcagggatgagcagaccgccagcgcctacagactgatggttgagcagcctagcgtgtccctgcctgccatctctcccgtggtcaatattgtgacaggcgccaccgtgtcctgggatggcaagggcctgaatatcatccacgtgggcgagacagaagtgatcctgggcgccgagaacaatcagctcatcgaactgaagaaggccatcttcgagaacctcgtgcggcagggcaagatcacctctctgcagaccccagagaaaaccgccatcagcacagagagctggcagcggtttcatcaggcctctccagaggatcaggccgaggctctggaacggtacaagaccatcgagccctacctgaacggacaccctcctgagaacgagacaatccccgccagaaccatcagacactggaaggccaagtacctgatcgccatccagaagtacggctgcggctatatcggcctgctgtcccacagaagcgtgaagggcaacagacagagaaagctgcccgaggacaccctggccatcatggaaaagttcattctcgaggactacgagacactgaagcagaaacggatgtgggaagtgcacgccgctctcgtgcgagcttgtgaagaagcaggcgttatcgcccctagctacaaggccttcaccaaagaggtgcagcggagaaccggctacgagcagaccaagaagagacaaggcagaagggccgcctaccagcacgagtccttttactgggaactcgccatcaccacacctcggcacggcgatagacctttcgagatcggccacatcgaccacaccgagctggatgtggaactcgtgtgtagcgacaccggcagaaacctgggtagaccttgggccacctttctggtggacgcctactctagaaggctgctggccgtgtacctgaccttcgacagcccaagctaccggtcctgtctcatggtgctgcggatctgcgtgaagaggcatggcagactgcctcagatcgtggtggtcgataatggcgccgagtttcacagcgtgtacttcgaaaccctgctggctaccttcgagtgcaccaagaagcagcggcctccagccaaggccagatttggcagcgtgtgcgagagactgttcggcaccagcaacacccagttcgtgcacaacctgctgggcaatacccagatcacccggaacgtgcggcaagtgaccaagagcgtgaaccccaagaacctggctgtgtggacactgggactgctgtacgagtacctgtgtgcctgggcctacgaagtgtacgataccgacgaacaccccgctctgtttcagagccctagggatgcctttgccgccggaatggctatcggcggatctcgggtgcacagaatgatcccttacgacgagaacttccagatcctgacactgcccaccacctccgagggcaaagccaaggtgcaagtcggcagaggcatcaagatcaactacatctactactggtccaacagcttcagggacccacagatcgagaacaccagcgtgcagatcagatacgaccccttcaacatcggaatcgcctacgccttcgtcagaggccagtgggtgcagtgcatcagccagtactacgccgaactccagggcagaagcgagaaagagctgaaactggccagcatcgagctgcggaagcactacagcaatcacacccagcagagcaaggtgtccgccaaaaacctggccgagttcctggcaagcgtggaagctcaagaagccctgctggaacagagaagctacgacgccgaagtgaaagaggtgttccgggtcatcgaaggcggcaaggccacaaccagcagaaatgaggaacccaagctgatccaagtgaccttcgccaacaccgacttccaggccgatgaggacgaagccatcgtgcctgaaacactggtggtgtacgaggaattcggaggatctggcggaggcggctctggcggcggtggaagcggaggacctaagaagaagagaaaagtctacccctacgacgtgccagactacgcctgactagcataaccccttggggcgc

SEQ ID NO: 347 - DNA, 2856 bp, Artificial Sequence
  Synthetic
gcgccccaaggggttatgctagtcaggcgtagtctggcacgtcgtaggggtagacttttctcttcttcttaggtcctccgcttccaccgccgccagagccgcctccgccagatcctccgaattcctcgtacaccaccagtgtttcaggcacgatggcttcgtcctcatcggcctggaagtcggtgttggcgaaggtcacttggatcagcttgggttcctcatttctgctggttgtggccttgccgccttcgatgacccggaacacctctttcacttcggcgtcgtagcttctctgttccagcagggcttcttgagcttccacgcttgccaggaactcggccaggtttttggcggacaccttgctctgctgggtgtgattgctgtagtgcttccgcagctcgatgctggccagtttcagctctttctcgcttctgccctggagttcggcgtagtactggctgatgcactgcacccactggcctctgacgaaggcgtaggcgattccgatgttgaaggggtcgtatctgatctgcacgctggtgttctcgatctgtgggtccctgaagctgttggaccagtagtagatgtagttgatcttgatgcctctgccgacttgcaccttggctttgccctcggaggtggtgggcagtgtcaggatctggaagttctcgtcgtaagggatcattctgtgcacccgagatccgccgatagccattccggcggcaaaggcatccctagggctctgaaacagagcggggtgttcgtcggtatcgtacacttcgtaggcccaggcacacaggtactcgtacagcagtcccagtgtccacacagccaggttcttggggttcacgctcttggtcacttgccgcacgttccgggtgatctgggtattgcccagcaggttgtgcacgaactgggtgttgctggtgccgaacagtctctcgcacacgctgccaaatctggccttggctggaggccgctgcttcttggtgcactcgaaggtagccagcagggtttcgaagtacacgctgtgaaactcggcgccattatcgaccaccacgatctgaggcagtctgccatgcctcttcacgcagatccgcagcaccatgagacaggaccggtagcttgggctgtcgaaggtcaggtacacggccagcagccttctagagtaggcgtccaccagaaaggtggcccaaggtctacccaggtttctgccggtgtcgctacacacgagttccacatccagctcggtgtggtcgatgtggccgatctcgaaaggtctatcgccgtgccgaggtgtggtgatggcgagttcccagtaaaaggactcgtgctggtaggcggcccttctgccttgtctcttcttggtctgctcgtagccggttctccgctgcacctctttggtgaaggccttgtagctaggggcgataacgcctgcttcttcacaagctcgcacgagagcggcgtgcacttcccacatccgtttctgcttcagtgtctcgtagtcctcgagaatgaacttttccatgatggccagggtgtcctcgggcagctttctctgtctgttgcccttcacgcttctgtgggacagcaggccgatatagccgcagccgtacttctggatggcgatcaggtacttggccttccagtgtctgatggttctggcggggattgtctcgttctcaggagggtgtccgttcaggtagggctcgatggtcttgtaccgttccagagcctcggcctgatcctctggagaggcctgatgaaaccgctgccagctctctgtgctgatggcggttttctctggggtctgcagagaggtgatcttgccctgccgcacgaggttctcgaagatggccttcttcagttcgatgagctgattgttctcggcgcccaggatcacttctgtctcgcccacgtggatgatattcaggcccttgccatcccaggacacggtggcgcctgtcacaatattgaccacgggagagatggcaggcagggacacgctaggctgctcaaccatcagtctgtaggcgctggcggtctgctcatccctgaagatcaggcatctctcaggctcagccagaggtgtggcgttcaggtcgatgtagatctgctcttggatgatcagggtgtagatgtcgtcgctcttggcgccggtgcttctgttcagcagctcggccagtgtaattccaggctggctagacacgatggccagcagagactgagcaatggcctcttccaccaccaggctctcgctgcggtagtaatcttccaggaactgcaggttccgctgcagcacccagttgatttcgcggtcgctgtggatccggtggtacaggccaaactgattggcgtacacctcgccaggcaggctgtgccactggttgttctcttcgctgtagaagtaccggttggggttcttctcggccagcttcttcagatcctgctcggtcttgcactcttcccagccggcgctattggtcctgatcacgaacagatcaggggtgtgcagcactcccagtcgtctgccattgctggcctggtagtccagcttgatctgaggaggctggtcgtagaattccagcacgtccttgtcgtgttccagctggtagatgatgggcagttccactctgtggctctcgaactggatggtcacgcccatctttctggaggggtatctgccggacacattcttgctgcctccgccaactcttctgctgggctcgctgctccgcactttctcgatgattttctggccggcttcagaaatctgcagccgggcgacccactgctcgaactgttccttcatggtggcgatctgagtccggtagcgctagcggatctgacggttca

SEQ ID NO: 348 - protein, 929 aa, Artificial Sequence; identical to SEQ ID NO: 97
  Synthetic

SEQ ID NO: 349 - protein, 593 aa, Artificial Sequence
  Synthetic
MPLHADPQHHETGPVAWAVEGQVHGQQPSRVGVHQKGGPRSTQVSAGVATHEFHIQLGVVDVADLERSIAVPRCGDGEFPVKGLVLVGGPSALSLLGLLVAGSPLHLFGEGLVARGDNACFFTSSHESGVHFPHPFLLQCLVVLENELFHDGQGVLGQLSLSVALHASVGQQADIAAAVLLDGDQVLGLPVSDGSGGDCLVLRRVSVQVGLDGLVPFQSLGLILWRGLMKPLPALCADGGFLWGLQRGDLALPHEVLEDGLLQFDELIVLGAQDHFCLAHVDDIQALAIPGHGGACHNIDHGRDGRQGHARLLNHQSVGAGGLLIPEDQASLRLSQRCGVQVDVDLLLDDQGVDVVALGAGASVQQLGQCNSRLARHDGQQRLSNGLFHHQALAAVVIFQELQVPLQHPVDFAVAVDPVVQAKLIGVHLARQAVPLVVLFAVEVPVGVLLGQLLQILLGLALFPAGAIGPDHEQIRGVQHSQSSAIAGLVVQLDLRRLVVEFQHVLVVFQLVDDGQFHSVALELDGHAHLSGGVSAGHILAASANSSAGLAAPHFLDDFLAGFRNLQPGDPLLELFLHGGDLSPVALADLTVH

SEQ ID NO: 350 - DNA, 47 bp, Artificial Sequence; identical to SEQ ID NO: 167
  Synthetic

SEQ ID NO: 351 - DNA, 47 bp, Artificial Sequence; identical to SEQ ID NO: 167
  Synthetic

SEQ ID NO: 352 - DNA, 47 bp, Artificial Sequence; identical to SEQ ID NO: 167
  Synthetic

SEQ ID NO: 353 - DNA, 44 bp, Artificial Sequence
  Synthetic
ctaccggactcagatcgccaccatgaccaagagcaccggctttc

SEQ ID NO: 354 - DNA, 21 bp, Artificial Sequence
  Synthetic
catggtggcgatctgagtccg

SEQ ID NO: 355 - DNA, 19 bp, Artificial Sequence
  Synthetic
ggaggatctggcggaggcg

SEQ ID NO: 356 - DNA, 46 bp, Artificial Sequence
  Synthetic
cagagccgcctccgccagatcctccagacacatcctgctgcactcc

SEQ ID NO: 357 - DNA, 47 bp, Artificial Sequence; identical to SEQ ID NO: 167
  Synthetic

SEQ ID NO: 358 - DNA, 32 bp, Artificial Sequence
  Synthetic
tgaggagtgcgccccaaggggttatgctagtc

SEQ ID NO: 359 - DNA, 1299 bp, Artificial Sequence
  Synthetic
cgctaccggactcagatcgccaccatgaccaagagcaccggctttccactggaactgctgaccagacctgccaccgagagactggcctacttcgagaactacaccgtggctcaccccagactgaaagaggtgtacgagatcctgatgcggacaatcgccgaacctgccggcgctagcttcatctttgtgtatggcgctagcggcgtgggcaagaccacactgagactgagagtggaacagaagctgaccgagctggccctgcctaagctggaaagcgatagagccagagtgcccgtcgtgggcattgaggctattgcccctgagagccggtacttcaactggaaagagtactacacacgggccctgatcaccctggaagaacccctgatcgaccacaagttcgactatggcgtgcggggcatcagcagagacaacttcggcaagatcaacgtggaaagcaaggtggtggcccctgctctgagaagggccctcgaaaatgccctgatccacagacaccccgacgtgttctttgtggacgaggcccagcactttggcaaggtggcctctggctacaagctgcaggatcagctggactgcctgaagtccctggccaacatgacaggcatcctgcactgtctgctgggcacctacgagctgctgaccttcagaaatctgagcggccagctgtctcggcggagcgtggacatccacttcagaagatactgcgccgactctcccgaggacgtgcaggcctttaaaagcgtgctgctgacattccagcagcatctgcctctggccgagacacccaatctggtggatcactgggagtacttctacgagagaaccctgggctgcatcggcaccctgaaggactggctgaagagagtgctgagcgacgccctggatagagaggccacaaccatcacactgaaggacctgcagaaaagggccctgagcgtggcccagtgccagaagatgttcaaagagatccaagagggcgaacgccagctgagcgaaacagaggccgatgtgcagaacctgagatctgcccttggcctgggagccaagcctatcgttctgcctgaggaaacccctaagaccaccagacctccaggcaaagtgggcaaaagaaagcccaagagggaccccatcggagtgcagcaggatgtgtctggaggatctggcggaggcggctctggcggcggtggaagcggaggacctaagaagaagagaaaagtctacccctacgacgtgccagactacgcctgactagcataaccccttggggcgcactcctca

SEQ ID NO: 360 - DNA, 1299 bp, Artificial Sequence
  Synthetic
tgaggagtgcgccccaaggggttatgctagtcaggcgtagtctggcacgtcgtaggggtagacttttctcttcttcttaggtcctccgcttccaccgccgccagagccgcctccgccagatcctccagacacatcctgctgcactccgatggggtccctcttgggctttcttttgcccactttgcctggaggtctggtggtcttaggggtttcctcaggcagaacgataggcttggctcccaggccaagggcagatctcaggttctgcacatcggcctctgtttcgctcagctggcgttcgccctcttggatctctttgaacatcttctggcactgggccacgctcagggcccttttctgcaggtccttcagtgtgatggttgtggcctctctatccagggcgtcgctcagcactctcttcagccagtccttcagggtgccgatgcagcccagggttctctcgtagaagtactcccagtgatccaccagattgggtgtctcggccagaggcagatgctgctggaatgtcagcagcacgcttttaaaggcctgcacgtcctcgggagagtcggcgcagtatcttctgaagtggatgtccacgctccgccgagacagctggccgctcagatttctgaaggtcagcagctcgtaggtgcccagcagacagtgcaggatgcctgtcatgttggccagggacttcaggcagtccagctgatcctgcagcttgtagccagaggccaccttgccaaagtgctgggcctcgtccacaaagaacacgtcggggtgtctgtggatcagggcattttcgagggcccttctcagagcaggggccaccaccttgctttccacgttgatcttgccgaagttgtctctgctgatgccccgcacgccatagtcgaacttgtggtcgatcaggggttcttccagggtgatcagggcccgtgtgtagtactctttccagttgaagtaccggctctcaggggcaatagcctcaatgcccacgacgggcactctggctctatcgctttccagcttaggcagggccagctcggtcagcttctgttccactctcagtctcagtgtggtcttgcccacgccgctagcgccatacacaaagatgaagctagcgccggcaggttcggcgattgtccgcatcaggatctcgtacacctctttcagtctggggtgagccacggtgtagttctcgaagtaggccagtctctcggtggcaggtctggtcagcagttccagtggaaagccggtgctcttggtcatggtggcgatctgagtccggtagcg

SEQ ID NO: 361 - protein, 264 aa, Artificial Sequence
  Synthetic
MLLECQQHAFKGLHVLGRVGAVSSEVDVHAPPRQLAAQISEGQQLVGAQQTVQDACHVGQGLQAVQLILQLVARGHLAKVLGLVHKEHVGVSVDQGIFEGPSQSRGHHLAFHVDLAEVVSADAPHAIVELVVDQGFFQGDQGPCVVLFPVEVPALRGNSLNAHDGHSGSIAFQLRQGQLGQLLFHSQSQCGLAHAASAIHKDEASAGRFGDCPHQDLVHLFQSGVSHGVVLEVGQSLGGRSGQQFQWKAGALGHGGDLSPVALA

SEQ ID NO: 362 - protein, 414 aa, Artificial Sequence; identical to SEQ ID NO: 98
  Synthetic